(19) United States
(12) Patent Application Publication
(10) Pub. No.: US 2003/0096239 A1
(43) Pub. Date: May 22, 2003

(54) Probes and decoder oligonucleotides

(76) Inventors: Kevin Gunderson, Encinitas, CA (US); Mark Chee, Del Mar, CA (US)

Correspondence Address: FLEHR HOHBACH TEST ALBRITTON & HERBERT LLP, Suite 3400, Four Embarcadero Center, San Francisco, CA, 94111, US

(21) Appl. No.: 09/940,185

(22) Filed: Aug. 27, 2001

Related U.S. Application Data
(60) Provisional application No. 60/227,948, filed on Aug. 25, 2000.
(60) Provisional application No. 60/228,854, filed on Aug. 29, 2000.

(51) Int. Cl. C12Q 1/68; C12M 1/34; B05D 3/00

(52) U.S. Cl. 435/6; 435/287.2; 427/2.11

(57) ABSTRACT

The present invention is directed to improved methods and compositions for the use of adapter sequences on arrays in a variety of multiplexed nucleic acid reactions, including synthesis reactions, amplification reactions, and genotyping reactions.

This application claims the benefit of U.S. Ser. Nos. 60/227,948 filed Aug. 25, 2000 and 60/228,854, filed Aug. 29, 2001, both of which are expressly incorporated herein by reference.

### FIELD OF THE INVENTION

The present invention is directed to methods and compositions for the use of adapter sequences on arrays in a variety of nucleic acid reactions, including synthesis reactions, amplification reactions, and genotyping reactions.

### BACKGROUND OF THE INVENTION

The detection of specific nucleic acids is an important tool for diagnostic medicine and molecular biology research. Gene probe assays currently play roles in identifying infectious organisms such as bacteria and viruses, in probing the expression of normal and mutant genes and identifying mutant genes such as oncogenes, in typing tissue for compatibility preceding tissue transplantation, in matching tissue or blood samples for forensic medicine, and for exploring homology among genes from different species.

Ideally, a gene probe assay should be sensitive, specific and easily automatable (for a review, see Nickerson, Current Opinion in Biotechnology 4:48-51 (1993)). The requirement for sensitivity (i.e. low detection limits) has been greatly alleviated by the development of the polymerase chain reaction (PCR) and other amplification technologies which allow researchers to amplify exponentially a specific nucleic acid sequence before analysis (for a review, see Abramson et al., Current Opinion in Biotechnology, 4:41-47 (1993)).

Specificity, in contrast, remains a problem in many currently available gene probe assays. The extent of molecular complementarity between probe and target defines the specificity of the interaction. Variations in the concentrations of probes, of targets and of salts in the hybridization medium, in the reaction temperature, and in the length of the probe may alter or influence the specificity of the probe/target interaction.

It may be possible under some circumstances to distinguish targets with perfect complementarity from targets with mismatches, although this is generally very difficult using traditional technology, since small variations in the reaction conditions will alter the hybridization. New experimental techniques for mismatch detection with standard probes include DNA ligation assays where single point mismatches prevent ligation and probe digestion assays in which mismatches create sites for probe cleavage.

Recent focus has been on the analysis of the relationship between genetic variation and phenotype by making use of polymorphic DNA markers. Previous work utilized short tandem repeats (STRs) as polymorphic positional markers; however, recent focus is on the use of single nucleotide polymorphisms (SNPs), which occur at an average frequency of more than 1 per kilobase in human genomic DNA. Some SNPs, particularly those in and around coding sequences, are likely to be the direct cause of therapeutically relevant phenotypic variants and/or disease predisposition. There are a number of well known polymorphisms that cause clinically important phenotypes; for example, the apoE2/3/4 variants are associated with different relative risk of Alzheimer's and other diseases (see Cordor et al., Science 261(1993). Multiplex PCR amplification of SNP loci with subsequent hybridization to oligonucleotide arrays has been shown to be an accurate and reliable method of simultaneously genotyping at least hundreds of SNPs; see Wang et al., Science, 280:1077 (1998); see also Schafer et al., Nature Biotechnology 16:33-39 (1998). The compositions of the present invention may easily be substituted for the arrays of the prior art.

There are a variety of particular techniques that are used to detect sequence, including mutations and SNPs. These include, but are not limited to, ligation based assays, cleavage based assays (mismatch and invasive cleavage such as Invader™), single base extension methods (see WO 92/15712, EP 0 371 437 B1, EP 0317 074 B1; Pastinen et al., Genome Res. 7:606-614 (1997); Syvänen, Clinica Chimica Acta 226:225-236 (1994); and WO 91/13075), and competitive probe analysis (e.g. competitive sequencing by hybridization; see below).

Oligonucleotide ligation amplification (“OLA”, which is referred as the ligation chain reaction (LCR) when two-stranded reactions or nested reactions are done) involves the ligation of two smaller probes into a single long probe, using the target sequence as the template. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; WO 97/31256 and WO 89/09835, all of which are incorporated by reference.

Invasive cleavage technology is based on structure-specific nucleases that cleave nucleic acids in a site-specific manner. Two probes are used: an “invader” probe and a “signalling” probe, that adjacently hybridize to a target sequence with a non-complementary overlap. The enzyme cleaves at the overlap due to its recognition of the “tail”, and releases the “tail” with a label. This can then be detected. The Invader™ technology is described in U.S. Pat. Nos. 5,846,717; 5,614,402; 5,719,028; 5,541,311; and 5,843,669, all of which are hereby incorporated by reference.

An additional technique utilizes sequencing by hybridization. For example, sequencing by hybridization has been described (Drmanac et al., Genomics 4:114 (1989); Koster et al., Nature Biotechnology 14:1123 (1996); U.S. Pat. Nos. 5,525,464; 5,202,231 and 5,695,940, among others, all of which are hereby expressly incorporated by reference in their entirety).

Sensitivity, i.e. detection limits, remain a significant obstacle in nucleic acid detection systems, and a variety of techniques have been developed to address this issue. Briefly, these techniques can be classified as either target amplification or signal amplification. Target amplification involves the amplification (i.e. replication) of the target sequence to be detected, resulting in a significant increase in the number of target molecules. Target amplification strategies include the polymerase chain reaction (PCR), strand displacement amplification (SDA), and nucleic acid sequence based amplification (NASBA).

Alternatively, rather than amplify the target, alternate techniques use the target as a template to replicate a signalling probe, allowing a small number of target molecules to result in a large number of signalling probes, that then can be detected. Signal amplification strategies include the ligase chain reaction (LCR), cycling probe technology (CPT), invasive cleavage techniques such as Invader™ technology, Q-Beta replicase (QβR) technology, and the use of “amplification probes” such as “branched DNA” that result in multiple label probes binding to a single target sequence.

The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, allelic PCR (see Newton et al. Nucl. Acid Res. 17:2503 91989); “reverse transcriptase PCR” or “RT-PCR”, “biotin capture PCR”, “vectorette PCR”. “panhandle PCR”, and “PCR select cDNA subtraction”, among others.

Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby incorporated by reference.

Nucleic acid sequence based amplification (NASBA) is generally described in U.S. Pat. No. 5,409,818 and “Profiting from Gene-based Diagnostics”, CTB International Publishing Inc., N.J., 1996, both of which are incorporated by reference.

Cycling probe technology (CPT) is a nucleic acid detection system based on signal or probe amplification rather than target amplification, such as is done in polymerase chain reactions (PCR). Cycling probe technology relies on a molar excess of labeled probe which contains a scissile linkage of RNA. Upon hybridization of the probe to the target, the resulting hybrid contains a portion of RNA:DNA. This area of RNA:DNA duplex is recognized by RNAseH and the RNA is excised, resulting in cleavage of the probe. The probe now consists of two smaller sequences which may be released, thus leaving the target intact for repeated rounds of the reaction. The unreacted probe is removed and the label is then detected. CPT is generally described in U.S. Pat. Nos. 5,011,769, 5,403,711, 5,660,988, and 4,876,187, and PCT published applications WO 95/05480, WO 95/1416, and WO 95/00667, all of which are specifically incorporated herein by reference.

The oligonucleotide ligation assay (OLA) involve the ligation of at least two smaller probes into a single long probe, using the target sequence as the template for the ligase. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; and WO 89/09835, all of which are incorporated by reference.

Invader™ technology is based on structure-specific polymerases that cleave nucleic acids in a site-specific manner. Two probes are used: an “invader” probe and a “signalling” probe, that adjacently hybridize to a target sequence with overlap. For mismatch discrimination, the invader technology relies on complementarity at the overlap position where cleavage occurs. The enzyme cleaves at the overlap, and releases the “ail” which may or may not be labeled. This can then be detected. The Invader™ technology is described in U.S. Pat. Nos. 5,846,717; 5,614,402; 5,719,028; 5,541,311; and 5,843,669, all of which are hereby incorporated by reference.

“Branched DNA” signal amplification relies on the synthesis of branched nucleic acids, containing a multiplicity of nucleic acid “arms” that function to increase the amount of label that can be put onto one probe. This technology is generally described in U.S. Pat. Nos. 5,681,702, 5,597,909, 5,545,730, 5,594,117, 5,591,584, 5,571,670, 5,580,731, 5,571,670, 5,591,584, 5,624,802, 5,635,352, 5,594,118, 5,359,100, 5,124,246 and 5,681,697, all of which are hereby incorporated by reference.

Similarily, dendrimers of nucleic acids serve to vastly increase the amount of label that can be added to a single molecule, using a similar idea but different compositions. This technology is as described in U.S. Pat. No. 5,175,270 and Nilsen et al., J. Theor. Biol. 187:273 (1997), both of which are incorporated herein by reference.

U.S. Ser. Nos. 09/189,543; 08/944,850; 09/033,462; 09/287,573; 09/151,877; 09/187,289 and 09/256,943; and PCT applications US98/09163 and US99/14387; US98/21193; US99/04473 and US98/05025, all of which are expressly incorporated by reference, describe novel compositions utilizing substrates with microsphere arrays, which allow for novel detection methods of nucleic acid hybridization.

The use of adapter-type sequences that allow the use of universal arrays has been described in limited contexts; see for example Chee et al., Nucl. Acid Res. 19:3301 (1991); Shoemaker et al., Nature Genetics 14:450 (1996); U.S. Pat. Nos. 5,494,810, 5,830,711, 6,027,889, 6,054,564, and 6,268,148; and EP 0 799 897 A1; WO 97/31256, all of which are expressly incorporated by reference.

Accordingly, it is an object of the present invention to provide methods for detecting nucleic acid reactions, and other target analytes, on arrays using adapter sequences.

### SUMMARY OF THE INVENTION

In accordance with the above objects, the invention also provides a method of detecting a target nucleic acid. The method comprises contacting the target nucleic acid with an adapter sequence such that the target nucleic acid is joined to the adapter sequence to form a modified target nucleic acid. In addition, the method comprises contacting the modified target nucleic acid with an array comprising a substrate with a surface comprising discrete sites and a population of microspheres comprising at least a first subpopulation comprising a first capture probe, such that the first capture probe and the modified target nucleic acid form a complex, wherein the microspheres are distributed on the surface, and detecting the presence fo the target nucleic acid. In addition the method comprises adding at least one decoding binding ligand to the array such that the identity of the target nucleic acid is determined. Preferably the adapter nucleic acids include a sequence as set forth in Table Table I, Table II, Table III or Table IV.

In addition the invention provides a method of making an array. The method comprises forming a surface comprising individual sites on a substrate, distributing microspheres on the surface such that the individual sites contain microspheres, wherein the microspheres comprise at least a first and a second subpopulation each comprising a capture probe, wherein the capture probe is complementary to an adapter sequence, the adapter sequence joined to a target nucleic acid, and an identifier binding ligand that will bind at least one decoder binding ligand such that the identification of the target nucleic acid is elucidated. Preferably the adapter nucleic acids include a sequence as set forth in Table I, Table II, Table III or Table IV.

In addition the invention provides a kit comprising at least one nucleic acid selected from the group consisting of the sequences set forth it Table I, Table II, Table III or Table IV. In one embodiment the invention provides a kit that includes a nucleic acid that includes a sequence as set forth in Table I, Table II, Table III or Table IV and at least a first universal priming sequence.

In addition the invention includes an array composition comprising a first population of microspheres comprising first and second subpopulations, wherein the first subpopulation includes a first nucleic acid selected from the sequences set forth in Table I, Table II, Table III or Table IV and the second subpopulation includes a second sequence selected from the sequences set forth in Table I, Table II, Table III or Table IV.

In addition the invention includes an array composition comprising a first sequence at a known location on a substrate, wherein the first sequence is selected from the sequences set forth in Table I, Table II, Table III or Table IV.

In addition the invention includes a method for making an array. The method includes distributing a population of microspheres on an substrate, wherein the population includes first and second subpopulations, wherein the first subpopulation includes a first sequence selected from the group consisting of the sequences set forth in Table I, Table II, Table III or Table IV and the second subpopulation includes a second sequence selected from the group consisting of the sequences set forth in Table I, Table II, Table III or Table IV.

In addition the method includes a method of immobilizing a target nucleic acid. The method includes hybridizing a first adapter probe with a first target nucleic acid, wherein the first adapter probe comprises a first domain that is complementary to the first target nucleic acid and a second domain, comprising a first sequence selected from the sequences set forth in Table I, Table II, Table III or Table IV to form a first hybridization complex. In addition the method includes contacting the first hybridization complex with a first capture probe immobilized on a first substrate, wherein the first capture probe is substantially complementary to the second domain of the first adapter probe.

In addition the invention includes a method of decoding an array composition comprising providing an array composition that includes a substrate with a surface comprising discrete sites and a population of microspheres comprising at least a first and a second subpopulation, wherein each subpopulation comprises a bioactive agent. The microspheres are distributed on the surface. The method further includes adding a plurality of decoding binding ligands to the array composition to identify the location of at least a plurality of the bioactive agents wherein at least a first decoder binding ligand comprises a sequence selected from the group consisting of the sequences of Table I, Table II, Table III or Table IV.

A method of detecting a target nucleic acid sequence, said method comprising attaching a first adapter nucleic acid to a first target nucleic acid sequence to form a modified first target nucleic acid sequence, wherein the first adapter nucleic acid includes a sequence selected from the sequences set forth in Table I, Table II, Table III or Table IV. The method further includes contacting the modified first target nucleic acid sequence with an array comprising a substrate with a patterned surface comprising discrete sites and a population of microspheres comprising at least a first subpopulation comprising a first capture probe, such that the first capture probe and the modified first target nucleic acid sequence form a hybridization complex; wherein the microspheres are distributed on the surface and detecting the presence of the modified first target nucleic acid sequence.

### DETAILED DESCRIPTION OF THE FIGURES

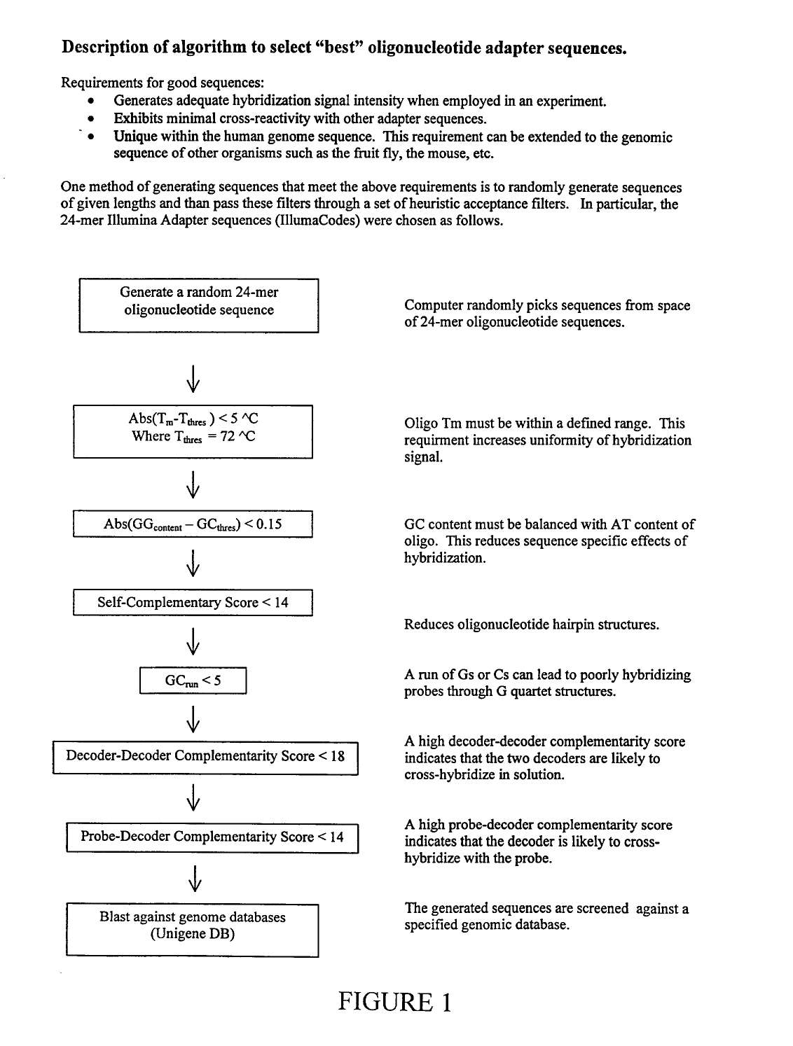

FIG. 1 depicts a method of selecting oligonucleotide sequences.

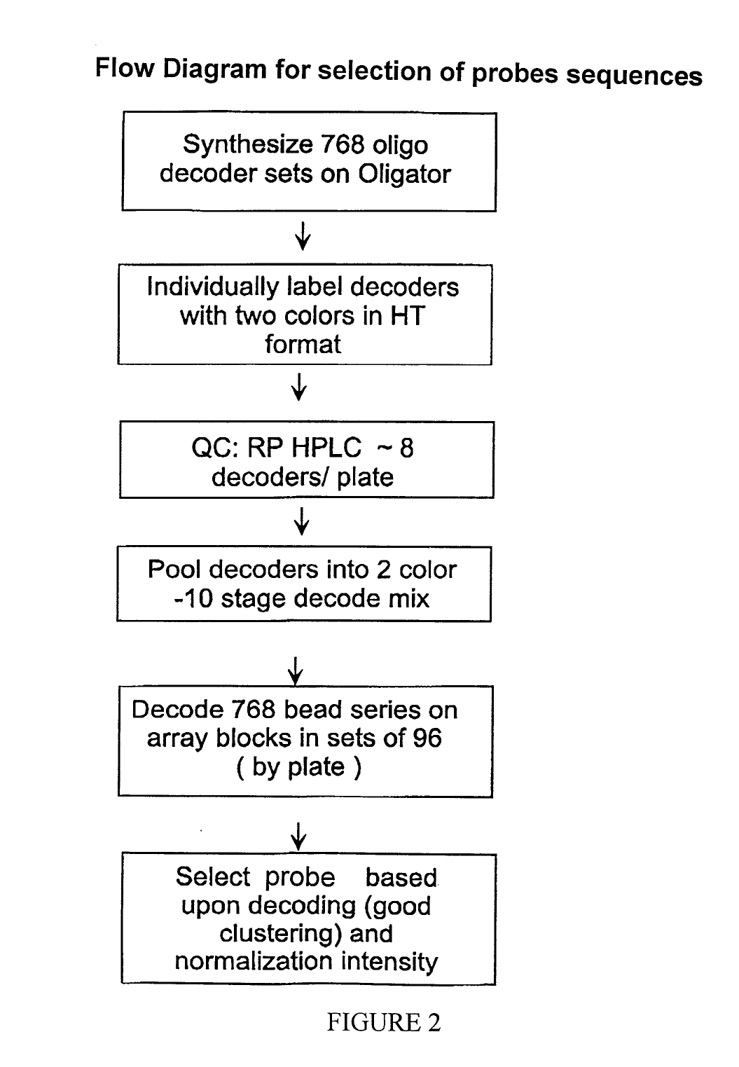

FIG. 2 depicts a scheme for selection of probes and decoder oligonucleotides.

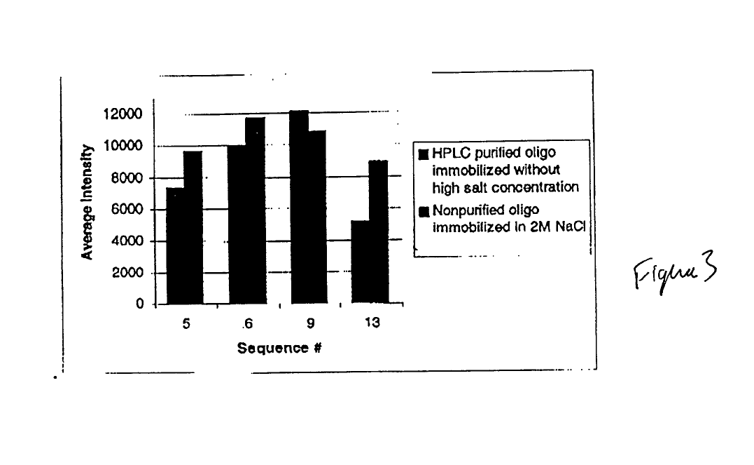

FIG. 3 demonstrates hybridization intensity comparison of immobilized beads using non-purified oligonucleotides with HPLC purified oligonucleotides.

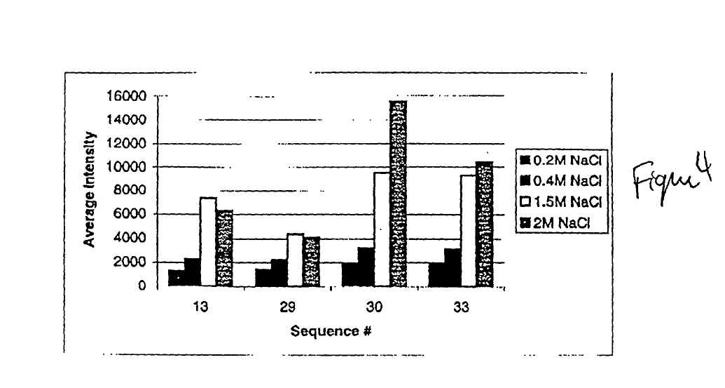

FIG. 4 depicts different oligonucleotide sequences immobilized onto silica beads at various salt concentration. Average intensity indicates hybridization intensity of beads in a BeadArray.

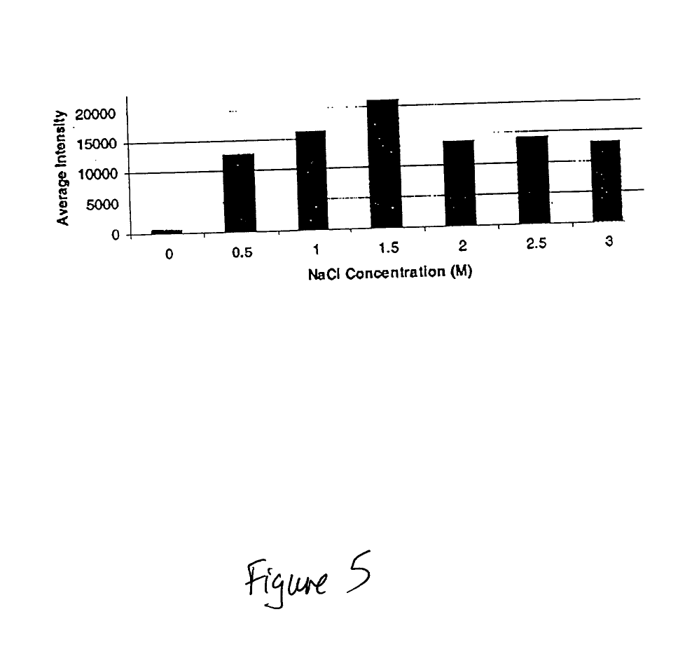

FIG. 5 depicts immobilization of oligonucleotides in increasing salt concentrations.

### DETAILED DESCRIPTION OF THE INVENTION

This invention is directed to the use of adapter sequences, and optionally capture extender probes, that allow the use of “universal” arrays. That is, a “universal” array is an array with a set of capture probes that will hybridize to adapter sequences, for use in any number of different reactions, including the binding of nucleic acid reactions and other target analytes comprising a nucleic acid adapter sequence that can hybridize to the array. In this way, a manufacturer of arrays can make one type of array that may be used in a variety of applications, thus reducing the manufacturing costs associated with the array. In addition, in the case of bead arrays, the decoding steps as outlined below can be simplified, as one set of decoding probes can be made.

In general, the use of adapter sequences can be described as follows for nucleic acid reactions. An adapter sequence can be added exogenously to a target nucleic acid sequence using any number of different techniques, including, but not limited to, amplification reactions as described in U.S. Ser. Nos. 09/425,633, filed Oct. 22, 1999; 09/513,362, filed Feb. 25, 2000; 09/517,945, filed Mar. 3, 2000; 09/535,854, filed Mar. 27, 2000; 09/553,993, filed Apr. 20, 2000; 09/556,463, filed Apr. 21, 2000; 60/135,051, filed May 20, 1999; 60/135,053, filed May 20, 1999; 60/135,123, filed May 20, 1999; 60/130,089, filed Apr. 20, 1999; 60/160,917, filed Oct. 22, 1999; 60/160,927, filed Oct. 22, 1999; 60/161,148, filed Oct. 22, 1999; and 60/244,119, filed Oct. 26, 2000 all of which are hereby incorporated by reference. In addition, the adapter can be added to an extension probe. The adapter sequence can then be used to target to its complementary capture probe on the surface.

Alternatively, the adapter sequences can be added to other target analytes, to generate unique and reproducible arrays of target analytes in a similar manner. By adding the nucleic acid to the target analyte (for example to an antibody in an immunoassay), the target analytes may then be arrayed.

Accordingly, the present invention provides methods for the detection of target analytes, particularly nucleic acid target sequences, in a sample. As will be appreciated by those in the art, the sample solution may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples; purified samples, such as purified genomic DNA, RNA, proteins, etc.; raw samples (bacteria, virus, genomic DNA, etc.; As will be appreciated by those in the art, virtually any experimental manipulation may have been done on the sample.

The present invention provides methods for the detection of target analytes, particularly nucleic acid target sequences, in a sample. By “target analyte” or “analyte” or grammatical equivalents herein is meant any molecule, compound or particle to be detected. As outlined below, target analytes preferably bind to binding ligands, as is more fully described below. As will be appreciated by those in the art, a large number of analytes may be detected using the present methods; basically, any target analyte for which a binding ligand, described below, may be made may be detected using the methods of the invention.

Suitable analytes include organic and inorganic molecules, including biomolecules. In a preferred embodiment, the analyte may be an environmental pollutant (including pesticides, insecticides, toxins, etc.); a chemical (including solvents, polymers, organic materials, etc.); therapeutic molecules (including therapeutic and abused drugs, antibiotics, etc.); biomolecules (including hormones, cytokines, proteins, lipids, carbohydrates, cellular membrane antigens and receptors (neural, hormonal, nutrient, and cell surface receptors) or their ligands, etc); whole cells (including procaryotic (such as pathogenic bacteria) and eukaryotic cells, including mammalian tumor cells); viruses (including retroviruses, herpesviruses, adenoviruses, lentiviruses, etc.); and spores; etc. Particularly preferred analytes are environmental pollutants; nucleic acids; proteins (including enzymes, antibodies, antigens, growth factors, cytokines, etc); therapeutic and abused drugs; cells; and viruses.

In a preferred embodiment, the target analyte is a protein. As will be appreciated by those in the art, there are a large number of possible proteinaceous target analytes that may be detected using the present invention. By “proteins” or grammatical equivalents herein is meant proteins, oligopeptides and peptides, derivatives and analogs, including proteins containing non-naturally occurring amino acids and amino acid analogs, and peptidomimetic structures. The side chains may be in either the (R) or the (S) configuration. In a preferred embodiment, the amino acids are in the (S) or L-configuration. As discussed below, when the protein is used as a binding ligand, it may be desirable to utilize protein analogs to retard degradation by sample contaminants.

Suitable protein target analytes include, but are not limited to, (1) immunoglobulins, particularly IgEs, IgGs and IgMs, and particularly therapeutically or diagnostically relevant antibodies, including but not limited to, for example, antibodies to human albumin, apolipoproteins (including apolipoprotein E), human chorionic gonadotropin, cortisol, α-fetoprotein, thyroxin, thyroid stimulating hormone (TSH), antithrombin, antibodies to pharmaceuticals (including antieptileptic drugs (phenytoin, primidone, carbariezepin, ethosuximide, valproic acid, and phenobarbitol), cardioactive drugs (digoxin, lidocaine, procainamide, and disopyramide), bronchodilators (theophylline), antibiotics (chloramphenicol, sulfonamides), antidepressants, immunosuppresants, abused drugs (amphetamine, methamphetamine, cannabinoids, cocaine and opiates) and antibodies to any number of viruses (including orthomyxoviruses, (e.g. influenza virus), paramyxoviruses (e.g respiratory syncytial virus, mumps virus, measles virus), adenoviruses, rhinoviruses, coronaviruses, reoviruses, togaviruses (e.g. rubella virus), parvoviruses, poxviruses (e.g. variola virus, vaccinia virus), enteroviruses (e.g. poliovirus, coxsackievirus), hepatitis viruses (including A, B and C), herpesviruses (e.g. Herpes simplex virus, varicella-zoster virus, cytomegalovirus, Epstein-Barr virus), rotaviruses, Norwalk viruses, hantavirus, arenavirus, rhabdovirus (e.g. rabies virus), retroviruses (including HIV, HTLV-I and -II), papovaviruses (e.g. papillomavirus), polyomaviruses, and picornaviruses, and the like), and bacteria (including a wide variety of pathogenic and non-pathogenic prokaryotes of interest including Bacillus; Vibrio, e.g. V. cholerae; Escherichia, e.g. Enterotoxigenic E. coli, Shigella, e.g. S. dysenteriae; Salmonella, e.g. S. typhi; Mycobacterium e.g. M. tuberculosis, M. leprae; Clostridium, e.g. C. botulinum, C. tetani, C. difficile, C.peffringens; Cornyebacterium, e.g. C. diphtheriae; Streptococcus, S. pyogenes, S. pneumoniae; Staphylococcus, e.g. S. aureus; Haemophilus, e.g. H. influenzae; Neisseria, e.g. N. meningitidis, N. gonorrhoeae; Yersinia, e.g. G. lambliaY. pestis, Pseudomonas, e.g. P. aeruginosa, P. putida; Chlamydia, e.g. C. trachomatis; Bordetella, e.g. B. pertussis; Treponema, e.g. T. palladium; and the like); (2) enzymes (and other proteins), including but not limited to, enzymes used as indicators of or treatment for heart disease, including creatine kinase, lactate dehydrogenase, aspartate amino transferase, troponin T, myoglobin, fibrinogen, cholesterol, triglycerides, thrombin, tissue plasminogen activator (tPA); pancreatic disease indicators including amylase, lipase, chymotrypsin and trypsin; liver function enzymes and proteins including cholinesterase, bilirubin, and alkaline phosphotase; aldolase, prostatic acid phosphatase, terminal deoxynucleotidyl transferase, and bacterial and viral enzymes such as HIV protease; (3) hormones and cytokines (many of which serve as ligands for cellular receptors) such as erythropoietin (EPO), thrombopoietin (TPO), the interleukins (including IL-1 through IL-17), insulin, insulin-like growth factors (including IGF-1 and -2), epidermal growth factor (EGF), transforming growth factors (including TGF-α and TGF-β), human growth hormone, transferrin, epidermal growth factor (EGF), low density lipoprotein, high density lipoprotein, leptin, VEGF, PDGF, ciliary neurotrophic factor, prolactin, adrenocorticotropic hormone (ACTH), calcitonin, human chorionic gonadotropin, cotrisol, estradiol, follicle stimulating hormone (FSH), thyroid-stimulating hormone (TSH), leutinzing hormone (LH), progeterone, testosterone, ; and (4) other proteins (including α-fetoprotein, carcinoembryonic antigen CEA.

In addition, any of the biomolecules for which antibodies may be detected may be detected directly as well; that is, detection of virus or bacterial cells, therapeutic and abused drugs, etc., may be done directly.

Suitable target analytes include carbohydrates, including but not limited to, markers for breast cancer (CA15-3, CA 549, CA 27.29), mucin-like carcinoma associated antigen (MCA), ovarian cancer (CA125), pancreatic cancer (DE-PAN-2), and colorectal and pancreatic cancer (CA 19, CA 50, CA242).

In a preferred embodiment, the target analyte (and various adapters and other probes of the invention), comprise nucleic acids. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate (Mag et al., Nucleic Acids Res. 19:1437 (1991); and U.S. Pat. No. 5,644,048), phosphorodithioate (Briu et al., J. Am. Chem. Soc. 111:2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). Other analog nucleic acids include those with positive backbones (Denpcy et al., Proc. Natl. Acad. Sci. USA 92:6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30:423 (1991); Letsinger et al., J. Am. Chem. Soc. 110:4470 (1988); Letsinger et al., Nucleoside & Nucleotide 13:1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Left. 4:395 (1994); Jeffs et al., J. Biomolecular NMR 34:17 (1994); Tetrahedron Left. 37:743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp169-176). Several nucleic acid analogs are described in Rawls, C & E News Jun. 2,1997 page 35. All of these references are hereby expressly incorporated by reference. These modifications of the ribose-phosphate backbone may be done to facilitate the addition of labels, alter the hybridization properties of the nucleic acids, or to increase the stability and half-life of such molecules in physiological environments.

As will be appreciated by those in the art, all of these nucleic acid analogs may find use in the present invention. In addition, mixtures of naturally occurring nucleic acids and analogs can be made. Alternatively, mixtures of different nucleic acid analogs, and mixtures of naturally occuring nucleic acids and analogs may be made.

Particularly preferred are peptide nucleic acids (PNA) which includes peptide nucleic acid analogs. These backbones are substantially non-ionic under neutral conditions, in contrast to the highly charged phosphodiester backbone of naturally occurring nucleic acids. This results in two advantages. First, the PNA backbone exhibits improved hybridization kinetics. PNAs have larger changes in the melting temperature (Tm) for mismatched versus perfectly matched basepairs. DNA and RNA typically exhibit a 2-4° C. drop in Tm for an internal mismatch. With the non-ionic PNA backbone, the drop is closer to 7-9° C. This allows for better detection of mismatches. Similarly, due to their non-ionic nature, hybridization of the bases attached to these backbones is relatively insensitive to salt concentration.

The nucleic acids may be single stranded or double stranded, as specified, or contain portions of both Id double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. A preferred embodiment utilizes isocytosine and isoguanine in nucleic acids designed to be complementary to other probes, rather than target sequences, as this reduces non-specific hybridization, as is generally described in U.S. Pat. No. 5,681,702. As used herein, the term “nucleoside” includes nucleotides as well as nucleoside and nucleotide analogs, and modified nucleosides such as amino modified nucleosides. In addition, “nucleoside” includes non-naturally occuring analog structures. Thus for example the individual units of a peptide nucleic acid, each containing a base, are referred to herein as a nucleoside.

In general, probes of the present invention (including adapter sequences and capture probes, described below) are designed to be complementary to a target sequence (either the target sequence of the sample or to other probe sequences, for example adapter sequences) such that hybridization of the target and the probes of the present invention occurs. This complementarity need not be perfect; there may be any number of base pair mismatches that will interfere with hybridization between the target sequence and the single stranded nucleic acids of the present invention. However, if the number of mutations is so great that no hybridization can occur under even the least stringent of hybridization conditions, the sequence is not a complementary target sequence. Thus, by “substantially complementary” herein is meant that the probes are sufficiently complementary to the target sequences to hybridize under the selected reaction conditions.

When nucleic acids are to be detected, they are referred to herein as “target nucleic acids” or “target sequences”. The term “target sequence” or “target nucleic acid” or grammatical equivalents herein means a nucleic acid sequence on a single strand of nucleic acid. The target sequence may be a portion of a gene, a regulatory sequence, genomic DNA, cDNA, RNA including mRNA and rRNA, or others. As is outlined herein, the target sequence may be a target sequence from a sample, or a derivative target such as a product of a reaction such as a detection sequence from an Invader™ reaction, a ligated probe from an OLA reaction, an extended probe from an SBE reaction, etc. It may be any length, with the understanding that longer sequences are more specific. As will be appreciated by those in the art, the complementary target sequence may take many forms. For example, it may be contained within a larger nucleic acid sequence, i.e. all or part of a gene or mRNA, a restriction fragment of a plasmid or genomic DNA, among others. As is outlined more fully below, probes are made to hybridize to target sequences to determine the presence or absence of the target sequence in a sample. Generally speaking, this term will be understood by those skilled in the art. The target sequence may also be comprised of different target domains; for example, a first target domain of the sample target sequence may hybridize to a capture probe, a second target domain may hybridize to a portion of a label probe, etc. The target domains may be adjacent or separated as indicated. Unless specified, the terms “first” and “second” are not meant to confer an orientation of the sequences with respect to the 5′-3′ orientation of the target sequence. For example, assuming a 5′-3′ orientation of the complementary target sequence, the first target domain may be located either 5′ to the second domain, or 3′ to the second domain. In addition, as will be appreciated by those in the art, the probes on the surface of the array (e.g. attached to the microspheres) may be attached in either orientation, either such that they have a free 3′ end or a free 5′ end.

As is more fully outlined below, the target sequence may comprise a position for which sequence information is desired, generally referred to herein as the “detection position” or “detection locus”. In a preferred embodiment, the detection position is a single nucleotide, although in some embodiments, rt may comprise a plurality of nucleotides, either contiguous with each other or separated by one or more nucleotides. By “plurality” as used herein is meant at least two. As used herein, the base which basepairs with a detection position base in a hybrid is termed a “readout position” or an “interrogation position”.

In some embodiments, as is outlined herein, the target sequence may not be the sample target sequence but instead is a product of a reaction herein, sometimes referred to herein as a “secondary” or “derivative” target sequence. Thus, for example, in SBE, the extended primer may serve as the target sequence; similarly, in invasive cleavage variations, the cleaved detection sequence may serve as the target sequence.

If required, the target sequence is prepared using known techniques. For example, the sample may be treated to lyse the cells, using known lysis buffers, electroporation, etc., with purification and/or amplification as needed, as will be appreciated by those in the art.

Once prepared, the target sequence can be used in a variety of reactions for a variety of reasons. For example, in a preferred embodiment, genotyping reactions are done. Similarly, these reactions can also be used to detect the presence or absence of a target sequence. Sequencing or amplification reactions are also preferred. In addition, in any reaction, quantitation of the amount of a target sequence may be done.

Furthermore, as outlined below for each reaction, many of these techniques may be used in a solution based assay, wherein the reaction is done in solution and a reaction product is bound to the array for subsequent detection, or in solid phase assays, where the reaction occurs on the surface and is detected.

In general, the present invention provides pairs of capture probes (nucleic acids that are attached to addresses on arrays) and adapter sequences (sequences that are either perfectly or substantially complementary to the capture probe sequences) that can be used in a wide variety of ways, to immobilize target nucleic acids (either primary targets, such as genomic DNA, mRNA or cDNA, or secondary targets such as amplicons from a nucleic acid amplification or extension reaction, as outlined herein) to the addresses of the array. Thus, all the sequences in the Tables include their complements, and either sequence can be used as a capture probe (e.g. spotted onto a surface or attached to a microsphere of an array) or as the adapter sequence that binds to the capture probe.

Accordingly, by “adapter sequences” or “adapters” or grammatical equivalents is meant a nucleic acid segment generally non-native or exogenous to a target molecule that is used to immobilize the target molecule to a solid support via binding to a capture probe sequence. In a preferred embodiment the adapter sequences and capture probes are selected from the sequences set forth in Table I, Table II, Table III or Table IV.

Table I includes the sequence of the preferred 4000 sequences labeled “Decoder (5′-3′)”, and inherent in this table are the complementary sequences as well. In addition, the invention includes oligonucleotides that are complementary to those depicted in Table 1.

Table II includes the sequence of the preferred adapter/capture probe sequences and their complementary sequence. Table 2 depicts a preferred subset of 3172 decoder oligonucleotides and their complementary probe oligonucleotides. Accordingly, the invention provides compositions comprising a sequence as outlined in Table 2. In addition, the invention provides a composition comprising a complementary binding pair as outlined in Table 2.

Table 3 includes a preferred subset of 768 decoder oligonucleotides and complementary probe sequences. In some embodiments it may be desirable to include a uniform base at a terminus of the oligonucleotide, such as a T at the 5′ end as depicted in Table 4. The inclusion of this uniform or constant base facilitates uniform labeling of the oligonucleotides.

These sequences are used as decoder probes, capture probes or adapter sequences as outlined in U.S .Ser. No. 09/344,526 and PCT/US99/14387, and U.S. Ser. Nos. 60/160,917 and 09/5656,463 all of which are expressly incorporated by reference in their entirety.

As will be appreciated by those in the art, the length of the capture probe/adapter sequences will vary, depending on the desired “strength” of binding and the number of different adapters desired. In a preferred embodiment, adapter sequences range from about 5 to about 500 basepairs in length, with from about 8 to about 100 being preferred, and from about 10 to about 50 being particularly preferred.

As will be appreciated by those in the art, it is desirable to have adapter sequences that do not have significant homology to naturally occurring target sequences, to avoid non-specific or erroneous binding of target sequences to the capture probes. Accordingly, preferred embodiments utilize some method to select useful adapter sequences. In a preferred embodiment the method is outlined in FIG. 1. Briefly, random 24-mer (or could be any desired length as outlined herein), sequences were assembled and subjected to certain defined screening procedures including such steps as requiring that the Tm of each of the sequence be within a pre-defined range. In addition the GC content must be balanced with the AT content and the self-complementarity must be minimized. In addition GC runs should be minimized, that is, runs of Gs or Cs should be reduced. In addition, decoder (adapter) to decoder (adapter) complementarity should be reduced so that the adapters do not hybridize with each other. Finally, the sequences are screened against a specified genomic database. In a preferred embodiment the adapters comprise at least one sequence selected from the sequences in Table I, Table II, Table III or Table IV.

In a preferred embodiment, the adapter sequences are chosen on the basis of a decoding step. As is more fully outlined below, a decoding step is used to decode random bead arrays. In this embodiment, a set of candidate capture probes is chosen; this may be done in a variety of ways. In a preferred embodiment, the sequences are generated randomly, each of a sufficient length to ensure a low probability of occurring naturally. In some embodiments, for example when the array will be used with a particular organism's genome (e.g. the human genome, the Drosophila genome, etc. ), the sequences are compared to the genome as a first filter, for example to remove sequences that would cross hybridize. Additionally, further filtering may be done using well-known methods, such as known methods for selecting good PCR primers. These techniques generally include steps that remove sequences that may have a propensity to form secondary structures or otherwise to cross-hybridize. Additionally, sequences that have extremes of melting temperatures can be optionally discarded, depending on the planned assay conditions.

Once a set of candidate capture probes is obtained, an array comprising the capture probes is made, and a matching set of decoding probes comprising the adapter sequences (e.g. the complements of the capture probes), as more fully outlined below, is made. Decoding then proceeds. Probes that do not hybridize well, for whatever reason, will not decode well, generally due to weak signals, and are generally discarded. Probes that cross-hybridize will also not decode well, as they will give ambiguous or mixed decoding signals. Only probes that hybridize sufficiently strongly and specifically will decode. Thus, by setting suitable thresholds for signal strength and signal purity, adapter sequences that perform according to specified criteria are identified. Additionally, by setting a range on signal strength, capture probe/adapter sequence pairs that perform similarly (but hybridize specifically) are identified. In a preferred embodiment, decoding reactions are repeated, under a variety of conditions, to test the robustness of the sequence pair.

Once identified, the adapter sequences are added to target sequences in a variety of ways, as will be appreciated by those in the art. In a preferred embodiment, nucleic acid amplification reactions are done, as is generally outlined in “Detection of Nucleic Acid Amplification Reactions Using Bead Arrays” and “Sequence Determination of Nucleic Acids using Arrays with Microspheres”, both of which were filed on Oct. 22, 1999, (U.S. Ser. Nos. 60/161,148 and 09/425,633, respectively), both of which are hereby incorporated by reference in their entirety. These may be either target amplification or signal amplification. In general, the techniques can be described as follows. Most amplification techniques require one or more primers hybridizing to all or part the target sequence (e.g. that hybridize to a target domain). The adapter sequences can be added to one or more of the primers (depending on the configuration/orientation of the system and need) and the amplification reactions are run. Thus, for example, PCR primers comprising at least one adapter sequence (and preferably one on each PCR primer) may be used; one or both of the ligation probes of an OLA or LCR reaction may comprise an adapter sequence; the sequencing primers for pyrosequencing, single-base extension, reversible chain termination, etc., reactions may comprise an adapter sequence; either the invader probe or the signalling probe of invasive cleavage reactions can comprise an adapter sequence; etc. Similarly, for signal detection techniques, the probes may comprise adapter sequences, with preferred methods utilizing removal of the unreacted probes. In addition, primers may include universal priming sequences. That is, the adapters may additionally contain universal priming sequences for universal amplification of products of any of the reactions described herein. Universal priming sequences are further outlined in 09/779376, filed Feb. 7, 2001; 09/779202, filed Feb. 7, 2001; 09/915231, filed Jul. 24, 2001; 60/180810, filed Feb. 7, 2000; and 60/297609, filed Jun. 11, 2001; and 60/311194 filed Aug. 9, 2001, all of which are expressly incorporated herein by reference.

In an alternative embodiment, non-nucleic acid reactions are used to add adapter sequences to the nucleic acid targets. For example, for the direct detection of non-amplified target sequences (e.g. genomic DNA samples, etc.) on universal arrays, non-amplification methods are required. In this embodiment, binding partner pairs or chemical methods may be used. For example, one member of a binding partner pair may be attached to the adapter sequence and the other member attached to the target sequence. For example, the binding partner be a hapten or antigen, which will bind its binding partner. For example, suitable binding partner pairs include, but are not limited to: antigens (such as proteins (including peptides)) and antibodies (including fragments thereof (FAbs, etc.)); proteins and small molecules, including biotin/streptavidin and digoxygenin and antibodies; enzymes and substrates or inhibitors; other protein-protein interacting pairs; receptor-ligands; and carbohydrates and their binding partners, are also suitable binding pairs. Nucleic acid-nucleic acid binding proteins pairs are also useful. In general, the smaller of the pair is attached to the NTP (or the probe) for incorporation into the extension primer. Preferred binding partner pairs include, but are not limited to, biotin (or imino-biotin) and streptavidin, digeoxinin and Abs, and Prolinx™ reagents.

In a preferred embodiment, chemical attachment methods are used. In this embodiment, chemical functional groups on each of the target sequences and adapter sequences are used. As is known in the art, this may be accomplished in a variety of ways. Preferred functional groups for attachment are amino groups, carboxy groups, oxo groups and thiol groups, with amino groups being particularly preferred. Using these functional groups, the two sequences are joined together; for example, amino groups on each nucleic acid may be attached, for example using linkers as are known in the art; for example, homo-or hetero-bifunctional linkers as are well known (see 1994 Pierce Chemical Company catalog, technical section on cross-linkers, pages 155-200, incorporated herein by reference).

In a preferred embodiment, aptamers are used in the system. Aptamers are nucleic acids that can be made to bind to virtually any target analyte; see Bock et al., Nature 355:564 (1992); Femulok et al., Current Op. Chem. Biol. 2:230 (1998); and U.S. Pat. Nos. 5,270,163, 5,475,096, 5,567,588, 5,595,877, 5,637,459, 5,683,867,5,705,337, and related patents, hereby incorporated by reference.

In a preferred embodiment, an array comprising capture probes that hybridize to adapter sequences is made, as outlined herein. In one embodiment aptamers, comprising adapter sequences, can be added. As will be appreciated by those in the art, the aptamers may be preassociated with their binding partners, e.g. target analytes, prior to introduction to the array, or not. In addition, the association between the adapter sequences on the aptamers and the capture probes can be made covalent, for example through the use of reactive groups (e.g. psoralen) and appropriate activation.

In addition, the present invention is directed to the use of adapter sequences to assemble arrays comprising other target analytes.

The adapter sequences may be chosen as outlined above. Preferably the adapters are selected from the sequences set forth in Table I, Table II, Table III or Table IV. These adapter sequences can then be added to the target analytes using a variety of techniques. In general, as described above, non-covalent attachment using binding partner pairs may be done, or covalent attachment using chemical moieties (including linkers).

Advantages of using adapters include but are not limited to, for example, the ability to create universal arrays. That is, a single array is utilized with each capture probe designed to hybridize with a specific adapter. The adapters are joined to any number of target analytes, such as nucleic acids, as is described herein. Thus, the same array is used for vastly different target analytes. Furthermore, hybridization of adapters with capture probes results in non-covalent attachment of the target nucleic acid to the address of the array (e.g. a microsphere in some embodiments). As such, the target nucleic/adapter hybrid is easily removed, and the microsphere/capture probe can be re-used. In addition, the construction of kits is greatly facilitated by the use of adapters. For example, arrays or microspheres can be prepared that comprise the capture probe; the adapters can be packaged along with the microspheres for attachment to any target analyte of interest. Thus, one need only attach the adapter to the target analyte and disperse on the array for the construction of an array of target analytes.

Accordingly the present invention provides kits comprising adapters. Preferably the kits include at least 1 nucleic acid sequence as set forth in Table 1. More preferably the kits include at least 10-25 nucleic acids, with at least 50 nucleic acids more preferred. Even more preferable are kits that include at least 100 nucleic acids with more than 1000 even more preferred and more than 2000 even more preferred.

It should also be noted that the sequences defined herein can also be used in “sandwich” assay formats, wherein a capture extender probe comprising a first domain that will hybridize to the capture probe and a second domain that has a target specific domain is used. The capture extender probe hybridizes both to the target sequence and the capture probe, thereby immobilizing the target sequence on the array.

Once the adapter sequences are associated with the target analyte, including target nucleic acids, the compositions are added to an array comprising addresses comprising capture probes. In one embodiment a plurality of hybrid adapter sequence/target analytes are pooled prior to addition to an array. All of the methods and compositions herein are drawn to compositions and methods for detecting the presence of target analytes, particularly nucleic acids, using adapter arrays.

Accordingly, the present invention provides array compositions comprising at least a first substrate with a surface comprising individual sites. The present system finds particular utility in array formats, i.e. wherein there is a matrix of capture probes (herein generally referred to “pads”, “addresses” or “micro-locations”). By “array” or “biochip” herein is meant a plurality of nucleic acids in an array format; the size of the array will depend on the composition and end use of the array. Nucleic acids arrays are known in the art, and can be classified in a number of ways; both ordered arrays (e.g. the ability to resolve chemistries at discrete sites), and random arrays are included. Ordered arrays include, but are not limited to, those made using photolithography techniques (Affymetrix GeneChip™), spotting techniques (Synteni and others), printing techniques (Hewlett Packard and Rosetta), three dimensional “gel pad” arrays, etc. In one embodiment the ordered arrays include arrays that contain nucleic acids at known locations. That is, the adapters or capture probes described herein are immobilized at known locations on a substrate. By “known” locations is meant a site that is known or has been known.

In addition, adapters find use “liquid arrays”. By “liquid arrays” is meant an array in solution for analysis, for example, by flow cytometry.

A preferred embodiment utilizes microspheres on a variety of substrates including fiber optic bundles, as are outlined in PCTs US98/21193, PCT US99/14387 and PCT US98/05025; WO98/50782; and U.S. Ser. Nos. 09/287,573, 09/151,877, 09/256,943, 09/316,154, 60/119,323, 09/315,584; all of which are expressly incorporated by reference. While much of the discussion below is directed to the use of microsphere arrays on fiber optic bundles, any array format of nucleic acids on solid supports may be utilized.

Arrays containing from about 2 different bioactive agents (e.g. different beads, when beads are used) to many millions can be made, with very large arrays being possible. Generally, the array will comprise from two to as many as a billion or more, depending on the size of the beads and the substrate, as well as the end use of the array, thus very high density, high density, moderate density, low density and very low density arrays may be made. Preferred ranges for very high density arrays are from about 10,000,000 to about 2,000,000,000, with from about 100,000,000 to about 1,000,000,000 being preferred (all numbers being in square cm). High density arrays range about 100,000 to about 10,000,000, with from about 1,000,000 to about 5,000,000 being particularly preferred. Moderate density arrays range from about 10,000 to about 100,000 being particularly preferred, and from about 20,000 to about 50,000 being especially preferred. Low density arrays are generally less than 10,000, with from about 1,000 to about 5,000 being preferred. Very low density arrays are less than 1,000, with from about 10 to about 1000 being preferred, and from about 100 to about 500 being particularly preferred. In some embodiments, the compositions of the invention may not be in array format; that is, for some embodiments, compositions comprising a single bioactive agent may be made as well. In addition, in some arrays, multiple substrates may be used, either of different or identical compositions. Thus for example, large arrays may comprise a plurality of smaller substrates.

In addition, one advantage of the present compositions is that particularly through the use of fiber optic technology, extremely high density arrays can be made. Thus for example, because beads of 200 μm or less (with beads of 200 nm possible) can be used, and very small fibers are known, it is possible to have as many as 40,000 or more (in some instances, 1 million) different elements (e.g. fibers and beads) in a 1 mm² fiber optic bundle, with densities of greater than 25,000,000 individual beads and fibers (again, in some instances as many as 50-100 million) per 0.5 cm² obtainable (4 million per square cm for 5 μ center-to-center and 100 million per square cm for 1 μ center-to-center).

By “substrate” or “solid support” or other grammatical equivalents herein is meant any material that can be modified to contain discrete individual sites appropriate for the attachment or association of beads and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, optical fiber bundles, and a variety of other polymers. In general, the substrates allow optical detection and do not themselves appreciably fluoresce.

Generally the substrate is flat (planar), although as will be appreciated by those in the art, other configurations of substrates may be used as well; for example, three dimensional configurations can be used, for example by embedding the beads in a porous block of plastic that allows sample access to the beads and using a confocal microscope for detection. Similarly, the beads may be placed on the inside surface of a tube, for flow-through sample analysis to minimize sample volume. Preferred substrates include optical fiber bundles as discussed below, and flat planar substrates such as glass, polystyrene and other plastics and acrylics.

In a preferred embodiment, the substrate is an optical fiber bundle or array, as is generally described in U.S. Ser. Nos. 08/944,850 and 08/519,062, PCT US98/05025, and PCT US98/09163, all of which are expressly incorporated herein by reference. Preferred embodiments utilize preformed unitary fiber optic arrays. By “preformed unitary fiber optic array” herein is meant an array of discrete individual fiber optic strands that are co-axially disposed and joined along their lengths. The fiber strands are generally individually clad. However, one thing that distinguished a preformed unitary array from other fiber optic formats is that the fibers are not individually physically manipulatable; that is, one strand generally cannot be physically separated at any point along its length from another fiber strand.

At least one surface of the substrate is modified to contain discrete, individual sites for later association of microspheres. These sites may comprise physically altered sites, i.e. physical configurations such as wells or small depressions in the substrate that can retain the beads, such that a microsphere can rest in the well, or the use of other forces (magnetic or compressive), or chemically altered or active sites, such as chemically functionalized sites, electrostatically altered sites, hydrophobically/ hydrophilically functionalized sites, spots of adhesive, etc.

The sites may be a pattern, i.e. a regular design or configuration, or randomly distributed. A preferred embodiment utilizes a regular pattern of sites such that the sites may be addressed in the X-Y coordinate plane. “Pattern” in this sense includes a repeating unit cell, preferably one that allows a high density of beads on the substrate. However, it should be noted that these sites may not be discrete sites. That is, it is possible to use a uniform surface of adhesive or chemical functionalities, for example, that allows the attachment of beads at any position. That is, the surface of the substrate is modified to allow attachment of the microspheres at individual sites, whether or not those sites are contiguous or non-contiguous with other sites. Thus, the surface of the substrate may be modified such that discrete sites are formed that can only have a single associated bead, or alternatively, the surface of the substrate is modified and beads may go down anywhere, but they end up at discrete

In a preferred embodiment, the surface of the substrate is modified to contain wells, i.e. depressions in the surface of the substrate. This may be done as is generally known in the art using a variety of techniques, including, but not limited to, photolithography, stamping techniques, molding techniques and microetching techniques. As will be appreciated by those in the art, the technique used will depend on the composition and shape of the substrate.

In a preferred embodiment, physical alterations are made in a surface of the substrate to produce the sites. In a preferred embodiment, the substrate is a fiber optic bundle and the surface of the substrate is a terminal end of the fiber bundle, as is generally described in 08/818,199 and 09/151,877, both of which are hereby expressly incorporated by reference. In this embodiment, wells are made in a terminal or distal end of a fiber optic bundle comprising individual fibers. In this embodiment, the cores of the individual fibers are etched, with respect to the cladding, such that small wells or depressions are formed at one end of the fibers. The required depth of the wells will depend on the size of the beads to be added to the wells.

Generally in this embodiment, the microspheres are non-covalently associated in the wells, although the wells may additionally be chemically functionalized as is generally described below, cross-linking agents may be used, or a physical barrier may be used, i.e. a film or membrane over the beads.

In a preferred embodiment, the surface of the substrate is modified to contain chemically modified sites, that can be used to attach, either covalently or non-covalently, the microspheres of the invention to the discrete sites or locations on the substrate. “Chemically modified sites” in this context includes, but is not limited to, the addition of a pattern of chemical functional groups including amino groups, carboxy groups, oxo groups and thiol groups, that can be used to covalently attach microspheres, which generally also contain corresponding reactive functional groups; the addition of a pattern of adhesive that can be used to bind the microspheres (either by prior chemical functionalization for the addition of the adhesive or direct addition of the adhesive); the addition of a pattern of charged groups (similar to the chemical functionalities) for the electrostatic attachment of the microspheres, i.e. when the microspheres comprise charged groups opposite to the sites; the addition of a pattern of chemical functional groups that renders the sites differentially hydrophobic or hydrophilic, such that the addition of similarly hydrophobic or hydrophilic microspheres under suitable experimental conditions will result in association of the microspheres to the sites on the basis of hydroaffinity. For example, the use of hydrophobic sites with hydrophobic beads, in an aqueous system, drives the association of the beads preferentially onto the sites. As outlined above, “pattern” in this sense includes the use of a uniform treatment of the surface to allow attachment of the beads at discrete sites, as well as treatment of the surface resulting in discrete sites. As will be appreciated by those in the art, this may be accomplished in a variety of ways.

In a preferred embodiment, the compositions of the invention further comprise a population of microspheres. By “population” herein is meant a plurality of beads as outlined above for arrays. Within the population are separate subpopulations, which can be a single microsphere or multiple identical microspheres. That is, in some embodiments, as is more fully outlined below, the array may contain only a single bead for each capture probe; preferred embodiments utilize a plurality of beads of each type.

By “microspheres” or “beads” or “particles” or grammatical equivalents herein is meant small discrete particles. The composition of the beads will vary, depending on the class of capture probe and the method of synthesis. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphite, titanium dioxide, latex or cross-linked dextrans such as Sepharose, cellulose, nylon, cross-linked micelles and Teflon may all be used. “Microsphere Detection Guide” from Bangs Laboratories, Fishers IN is a helpful guide.

The beads need not be spherical; irregular particles may be used. In addition, the beads may be porous, thus increasing the surface area of the bead available for either capture probe attachment or tag attachment. The bead sizes range from nanometers, i.e. 100 nm, to millimeters, i.e. 1 mm, with beads from about 0.2 micron to about 200 microns being preferred, and from about 0.5 to about 5 micron being particularly preferred, although in some embodiments smaller beads may be used.

It should be noted that a key component of this embodiment of the invention is the use of a substrate/bead pairing that allows the association or attachment of the beads at discrete sites on the surface of the substrate, such that the beads do not move during the course of the assay.

Each microsphere comprises a capture probe, although as will be appreciated by those in the art, there may be some microspheres which do not contain a capture probe, depending on the synthetic methods. Alternatively, some have more than one capture probe.

Attachment of the nucleic acids may be done in a variety of ways, as will be appreciated by those in the art, including, but not limited to, chemical or affinity capture (for example, including the incorporation of derivatized nucleotides such as AminoLink or biotinylated nucleotides that can then be used to attach the nucleic acid to a surface, as well as affinity capture by hybridization), cross-linking, and electrostatic attachment, etc. In a preferred embodiment, affinity capture is used to attach the nucleic acids to the beads. For example, nucleic acids can be derivatized, for example with one member of a binding pair, and the beads derivatized with the other member of a binding pair. Suitable binding pairs are as described herein for IBUDBL pairs. For example, the nucleic acids may be biotinylated (for example using enzymatic incorporate of biotinylated nucleotides, for by photoactivated cross-linking of biotin). Biotinylated nucleic acids can then be captured on streptavidin-coated beads, as is known in the art. Similarly, other hapten-receptor combinations can be used, such as digoxigenin and anti-digoxigenin antibodies. Alternatively, chemical groups can be added in the form of derivatized nucleotides, that can them be used to add the nucleic acid to the surface.

Preferred attachments are covalent, although even relatively weak interactions (i.e. non-covalent) can be sufficient to attach a nucleic acid to a surface, if there are multiple sites of attachment per each nucleic acid. Thus, for example, electrostatic interactions can be used for attachment, for example by having beads carrying the opposite charge to the bioactive agent.

Similarly, affinity capture utilizing hybridization can be used to attach nucleic acids to beads. For example, as is known in the art, polyA+RNA is routinely captured by hybridization to oligo-dT beads; this may include oligo-dT capture followed by a cross-linking step, such as psoralen crosslinking). If the nucleic acids of interest do not contain a polyA tract, one can be attached by polymerization with terminal transferase, or via ligation of an oligoA linker, as is known in the art.

Alternatively, chemical crosslinking may be done, for example by photoactivated crosslinking of thymidine to reactive groups, as is known in the art.

In a preferred embodiment, each bead comprises a single type of capture probe, although a plurality of individual capture probes are preferably attached to each bead. Similarly, preferred embodiments utilize more than one microsphere containing a unique capture probe; that is, there is redundancy built into the system by the use of subpopulations of microspheres, each microsphere in the subpopulation containing the same capture probe.

In an alternative embodiment, each bead comprises a plurality of different capture probes.

As will be appreciated by those in the art, the capture probes may either be synthesized directly on the beads, or they may be made and then attached after synthesis. In a preferred embodiment, linkers are used to attach the capture probes to the beads, to allow both good attachment, sufficient flexibility to allow good interaction with the target molecule, and to avoid undesirable binding reactions.

In a preferred embodiment, the capture probes are synthesized directly on the beads. As is known in the art, many classes of chemical compounds are currently synthesized on solid supports, such as peptides, organic moieties, and nucleic acids. It is a relatively straightforward matter to adjust the current synthetic techniques to use beads.

In a preferred embodiment, the capture probes are synthesized first, and then covalently attached to the beads. As will be appreciated by those in the art, this will be done depending on the composition of the capture probes and the beads. The functionalization of solid support surfaces such as certain polymers with chemically reactive groups such as thiols, amines, carboxyls, etc. is generally known in the art. Accordingly, “blank” microspheres may be used that have surface chemistries that facilitate the attachment of the desired functionality by the user. Some examples of these surface chemistries for blank microspheres include, but are not limited to, amino groups including aliphatic and aromatic amines, carboxylic acids, aldehydes, amides, chloromethyl groups, hydrazide, hydroxyl groups, sulfonates and sulfates.

In a preferred embodiment the attachment of nucleic acids to substrates includes contacting the oligonucleotide and the solid support in the presence of high salt concentrations. As is appreciated by those skilled in the art, salt includes, but is not limited to sodium chloride, potassium chloride, calcium chloride, magnesium chloride, lithium chloride, rubidium chloride, cesium chloride, barium chloride and the like. In a preferred embodiment, salt as used in the invention includes sodium chloride.

By high salt concentrations is meant salt that is more concentrated than about 0.1 M salt. In a preferred embodiment, by high salt concentrations is meant greater than about 0.2 M salt. In a particularly preferred embodiment, high salt concentrations include from about 0.5 to 3 M salt, with about 1 M to 2 M being most preferred.

By solid support or other grammatical equivalents herein is meant any material that can be modified to contain oligonucleotides. As will be appreciated by those in the art, the number of possible solid supports is very large. Possible solid supports include, but are not limited to beads, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, optical fiber bundles, and a variety of other polymers.

Once formed, the support containing the oligonucleotides finds use in a variety of systems including decoding arrays as described in more detail in U.S. Ser. No. 09/344,526, and U.S. Ser. No. 09/574,117, both of which are expressly incorporated herein by reference. In addition, the support containing the oligonucleotides finds use in microfluidic systems as described in U.S. Ser. No. 09/306,369 which is expressly incorporated herein by reference. In addition, the support containing the oligonucleotides finds use in composite array systems as described in U.S. Ser. No. 09/606,369, which is expressly incorporated herein by reference. In addition the support containing the oligonucleotides finds use in a variety of assays as outlined in more detail in U.S. Ser. Nos. 09/513,362, 09/517,945, 09/535,854, 60/160,917, 60/180,810, 60/182,955, and 09/566,463, all of which are expressly incorporated herein by reference in their entirety. In addition, the support containing the oligonucleotides finds use in array based sensors as described in more detail in 09/287,573, 09/260,963, 09/450,829, 09/151,877, 09/187,289 and 08/519,062, all of which are expressly incorporated herein by reference in their entirety.

Accordingly the invention provides a method of attaching oligonucleotides to a solid support. The method includes contacting the oligonucleotides with the support in the presence of high salt as described herein. Once attached, as discussed in the examples, the attached oligonucleotides readily hybridize to targets, probes and the like. Attachment of crude oligonucleotides in the presence of high salt is as efficient as attaching purified oligonucleotides. Thus, the invention also contemplates a method of attachment of oligonucleotides to a solid support without prior purification of the oligonucleotides. Again, the method includes contacting the crude oligonucleotides with a solid support in the presence of high salt as described herein.

The capture probes are designed to be substantially complementary to the adapter sequences, to allow for a minimum of cross reactivity.

When microsphere arrays are used, an encoding/decoding system must be used. That is, since the beads are generally put onto the substrate randomly, there are several ways to correlate the functionality on the bead with its location, including the incorporation of unique optical signatures, generally fluorescent dyes, that could be used to identify the chemical functionality on any particular bead. This allows the synthesis of the candidate agents (i.e. compounds such as nucleic acids and antibodies) to be divorced from their placement on an array, i.e. the candidate agents may be synthesized on the beads, and then the beads are randomly distributed on a patterned surface. Since the beads are first coded with an optical signature, this means that the array can later be “decoded”, i.e. after the array is made, a correlation of the location of an individual site on the array with the bead or candidate agent at that particular site can be made. This means that the beads may be randomly distributed on the array, a fast and inexpensive process as compared to either the in situ synthesis or spotting techniques of the prior art.

However, the drawback to these methods is that for a large array, the system requires a large number of different optical signatures, which may be difficult or time-consuming to utilize. Accordingly, the present invention provides several improvements over these methods, generally directed to methods of coding and decoding the arrays. That is, as will be appreciated by those in the art, the placement of the capture probes is generally random, and thus a coding/decoding system is required to identify the probe at each location in the array. This may be done in a variety of ways, as is more fully outlined below, and generally includes: a) the use a decoding binding ligand (DBL), generally directly labeled, that binds to either the capture probe or to identifier binding ligands (IBLs) attached to the beads; b) positional decoding, for example by either targeting the placement of beads (for example by using photoactivatible or photocleavable moieties to allow the selective addition of beads to particular locations), or by using either sub-bundles or selective loading of the sites, as are more fully outlined below; c) selective decoding, wherein only those beads that bind to a target are decoded; or d) combinations of any of these. In some cases, as is more fully outlined below, this decoding may occur for all the beads, or only for those that bind a particular target sequence. Similarly, this may occur either prior to or after addition of a target sequence. In addition, as outlined herein, the target sequences detected may be either a primary target sequence (e.g. a patient sample), or a reaction product from one of the methods described herein (e.g. an extended SBE probe, a ligated probe, a cleaved signal probe, etc.).

Once the identity (i.e. the actual agent) and location of each microsphere in the array has been fixed, the array is exposed to samples containing the target sequences, although as outlined below, this can be done prior to or during the analysis as well. The target sequences can hybridize (either directly or indirectly) to the capture probes as is more fully outlined below, and results in a change in the optical signal of a particular bead.

In the present invention, “decoding” may not rely on the use of optical signatures, but rather on the use of decoding binding ligands that are added during a decoding step. The decoding binding ligands will bind either to a distinct identifier binding ligand partner that is placed on the beads, or to the capture probe itself. In this embodiment the decoding binding ligand either is complementary to the capture probe. In this embodiment the decoding binding ligand has the sequence of the adapter that also binds to the capture probe. In a preferred embodiment the decoder binding ligand is a nucleic acid that has the sequence of at least one of the nucleic acids set forth in Table 1.

The decoding binding ligands are either directly or indirectly labeled, and thus decoding occurs by detecting the presence of the label. By using pools of decoding binding ligands in a sequential fashion, it is possible to greatly minimize the number of required decoding steps.

In some embodiments, the microspheres may additionally comprise identifier binding ligands for use in certain decoding systems. By “identifier binding ligands” or “IBLs” herein is meant a compound that will specifically bind a corresponding decoder binding ligand (DBL) to facilitate the elucidation of the identity of the capture probe attached to the bead. That is, the IBL and the corresponding DBL form a binding partner pair. By “specifically bind” herein is meant that the IBL binds its DBL with specificity sufficient to differentiate between the corresponding DBL and other DBLs (that is, DBLs for other IBLs), or other components or contaminants of the system. The binding should be sufficient to remain bound under the conditions of the decoding step, including wash steps to remove non-specific binding. In some embodiments, for example when the IBLs and corresponding DBLs are proteins or nucleic acids, the dissociation constants of the IBL to its DBL will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

IBL-DBL binding pairs are known or can be readily found using known techniques. For example, when the IBL is a protein, the DBLs include proteins (particularly including antibodies or fragments thereof (FAbs, etc.)) or small molecules, or vice versa (the IBL is an antibody and the DBL is a protein). Metal ion-metal ion ligands or chelators pairs are also useful. Antigen-antibody pairs, enzymes and substrates or inhibitors, other protein-protein interacting pairs, receptor-ligands, complementary nucleic acids, and carbohydrates and their binding partners are also suitable binding pairs. Nucleic acid—nucleic acid binding proteins pairs are also useful. Similarly, as is generally described in U.S. Pat. Nos. 5,270,163, 5,475,096, 5,567,588, 5,595,877, 5,637,459, 5,683,867, 5,705,337, and related patents, hereby incorporated by reference, nucleic acid “aptamers” can be developed for binding to virtually any target; such an aptamer-target pair can be used as the IBL-DBL pair. Similarly, there is a wide body of literature relating to the development of binding pairs based on combinatorial chemistry methods.

In a preferred embodiment, the IBL is a molecule whose color or luminescence properties change in the presence of a selectively-binding DBL. For example, the IBL may be a fluorescent pH indicator whose emission intensity changes with pH. Similarly, the IBL may be a fluorescent ion indicator, whose emission properties change with ion concentration.

Alternatively, the IBL is a molecule whose color or luminescence properties change in the presence of various solvents. For example, the IBL may be a fluorescent molecule such as an ethidium salt whose fluorescence intensity increases in hydrophobic environments. Similarly, the IBL may be a derivative of fluorescein whose color changes between aqueous and nonpolar solvents.

In one embodiment, the DBL may be attached to a bead, i.e. a “decoder bead”, that may carry a label such as a fluorophore.

In a preferred embodiment, the IBL-DBL pair comprise substantially complementary single-stranded nucleic acids. In this embodiment, the binding ligands can be referred to as “identifier probes” and “decoder probes”. Generally, the identifier and decoder probes range from about 4 basepairs in length to about 1000, with from about 6 to about 100 being preferred, and from about 8 to about 40 being particularly preferred. What is important is that the probes are long enough to be specific, i.e. to distinguish between different IBL-DBL pairs, yet short enough to allow both a) dissociation, if necessary, under suitable experimental conditions, and b) efficient hybridization.

In a preferred embodiment, as is more fully outlined below, the IBLs do not bind to DBLs. Rather, the IBLs are used as identifier moieties (“IMs”) that are identified directly, for example through the use of mass spectroscopy.

Alternatively, in a preferred embodiment, the IBL and the capture probe are the same moiety; thus, for example, as outlined herein, particularly when no optical signatures are used, the capture probe can serve as both the identifier and the agent. For example, in the case of nucleic acids, the bead-bound probe (which serves as the capture probe) can also bind decoder probes, to identify the sequence of the probe on the bead. Thus, in this embodiment, the DBLs bind to the capture probes.

In one embodiment, the microspheres may contain an optical signature. That is, as outlined in U.S. Ser. Nos. 08/818,199 and 09/151,877, previous work had each subpopulation of microspheres comprising a unique optical signature or optical tag that is used to identify the unique capture probe of that subpopulation of microspheres; that is, decoding utilizes optical properties of the beads such that a bead comprising the unique optical signature may be distinguished from beads at other locations with different optical signatures. Thus the previous work assigned each capture probe a unique optical signature such that any microspheres comprising that capture probe are identifiable on the basis of the signature. These optical signatures comprised dyes, usually chromophores or fluorophores, that were entrapped or attached to the beads themselves. Diversity of optical signatures utilized different fluorochromes, different ratios of mixtures of fluorochromes, and different concentrations (intensities) of fluorochromes.

In a preferred embodiment, the present invention does not rely solely on the use of optical properties to decode the arrays. However, as will be appreciated by those in the art, it is possible in some embodiments to utilize optical signatures as an additional coding method, in conjunction with the present system. Thus, for example, as is more fully outlined below, the size of the array may be effectively increased while using a single set of decoding moieties in several ways, one of which is the use of optical signatures one some beads. Thus, for example, using one “set” of decoding molecules, the use of two populations of beads, one with an optical signature and one without, allows the effective doubling of the array size. The use of multiple optical signatures similarly increases the possible size of the array.

In a preferred embodiment, each subpopulation of beads comprises a plurality of different IBLs. By using a plurality of different IBLs to encode each capture probe, the number of possible unique codes is substantially increased. That is, by using one unique IBL per capture probe. the size of the array will be the number of unique IBLs (assuming no “reuse” occurs, as outlined below). However, by using a plurality of different IBLs per bead, n, the size of the array can be increased to 2ⁿ, when the presence or absence of each IBL is used as the indicator. For example, the assignment of 10 IBLs per bead generates a bit binary code, where each bit can be designated as “1” (IBL is present) or “0” (IBL is absent). A 10 bit binary code has 2¹⁰ possible variants However, as is more fully discussed below, the size of the array may be further increased if another parameter is included such as concentration or intensity; thus for example, if two different concentrations of the IBL are used, then the array size increases as 3ⁿ. Thus, in this embodiment, each individual capture probe in the array is assigned a combination of IBLs, which can be added to the beads prior to the addition of the capture probe, after, or during the synthesis of the capture probe, i.e. simultaneous addition of IBLs and capture probe components.

Alternatively, the combination of different IBLs can be used to elucidate the sequence of the nucleic acid. Thus, for example, using two different IBLs (IBL1 and IBL2), the first position of a nucleic acid can be elucidated: for example, adenosine can be represented by the presence of both IBL1 and IBL2; thymidine can be represented by the presence of IBL1 but not IBL2, cytosine can be represented by the presence of IBL2 but not IBL1, and guanosine can be represented by the absence of both. The second position of the nucleic acid can be done in a similar manner using IBL3 and IBL4; thus, the presence of IBL1, IBL2, IBL3 and IBL4 gives a sequence of AA; IBL1, IBL2, and IBL3 shows the sequence AT; IBL1, IBL3 and IBL4 gives the sequence TA, etc. The third position utilizes IBL5 and IBL6, etc. In this way, the use of 20 different identifiers can yield a unique code for every possible 10-mer.

In this way, a sort of “bar code” for each sequence can be constructed; the presence or absence of each distinct IBL will allow the identification of each capture probe.

In addition, the use of different concentrations or densities of IBLs allows a “reuse” of sorts. If, for example, the bead comprising a first agent has a 1× concentration of IBL, and a second bead comprising a second agent has a 1× concentration of IBL, using saturating concentrations of the corresponding labelled DBL allows the user to distinguish between the two beads.

Once the microspheres comprising the capture probes are generated, they are added to the substrate to form an array. It should be noted that while most of the methods described herein add the beads to the substrate prior to the assay, the order of making, using and decoding the array can vary. For example, the array can be made, decoded, and then the assay done. Alternatively, the array can be made, used in an assay, and then decoded; this may find particular use when only a few beads need be decoded. Alternatively, the beads can be added to the assay mixture, i.e. the sample containing the target sequences, prior to the addition of the beads to the substrate; after addition and assay, the array may be decoded. This is particularly preferred when the sample comprising the beads is agitated or mixed; this can increase the amount of target sequence bound to the beads per unit time, and thus (in the case of nucleic acid assays) increase the hybridization kinetics. This may find particular use in cases where the concentration of target sequence in the sample is low; generally, for low concentrations, long binding times must be used.

In general, the methods of making the arrays and of decoding the arrays is done to maximize the number of different candidate agents that can be uniquely encoded. The compositions of the invention may be made in a variety of ways. In general, the arrays are made by adding a solution or slurry comprising the beads to a surface containing the sites for attachment of the beads. This may be done in a variety of buffers, including aqueous and organic solvents, and mixtures. The solvent can evaporate, and excess beads are removed.

In a preferred embodiment, when non-covalent methods are used to associate the beads with the array, a novel method of loading the beads onto the array is used. This method comprises exposing the array to a solution of particles (including microspheres and cells) and then applying energy, e.g. agitating or vibrating the mixture. This results in an array comprising more tightly associated particles, as the agitation is done with sufficient energy to cause weakly-associated beads to fall off (or out, in the case of wells). These sites are then available to bind a different bead. In this way, beads that exhibit a high affinity for the sites are selected. Arrays made in this way have two main advantages as compared to a more static loading: first of all, a higher percentage of the sites can be filled easily, and secondly, the arrays thus loaded show a substantial decrease in bead loss during assays. Thus, in a preferred embodiment, these methods are used to generate arrays that have at least about 50% of the sites filled, with at least about 75% being preferred, and at least about 90% being particularly preferred. Similarly, arrays generated in this manner preferably lose less than about 20% of the beads during an assay, with less than about 10% being preferred and less than about 5% being particularly preferred.

In this embodiment, the substrate comprising the surface with the discrete sites is immersed into a solution comprising the particles (beads, cells, etc.). The surface may comprise wells, as is described herein, or other types of sites on a patterned surface such that there is a differential affinity for the sites. This differnetial affinity results in a competitive process, such that particles that will associate more tightly are selected. Preferably, the entire surface to be “loaded” with beads is in fluid contact with the solution. This solution is generally a slurry ranging from about 10,000:1 beads:solution (vol:vol) to 1:1. Generally, the solution can comprise any number of reagents, including aqueous buffers, organic solvents, salts, other reagent components, etc. In addition, the solution preferably comprises an excess of beads; that is, there are more beads than sites on the array. Preferred embodiments utilize two-fold to billion-fold excess of beads.

The immersion can mimic the assay conditions; for example, if the array is to be “dipped” from above into a microtiter plate comprising samples, this configuration can be repeated for the loading, thus minimizing the beads that are likely to fall out due to gravity.

Once the surface has been immersed, the substrate, the solution, or both are subjected to a competitive process, whereby the particles with lower affinity can be disassociated from the substrate and replaced by particles exhibiting a higher affinity to the site. This competitive process is done by the introduction of energy, in the form of heat, sonication, stirring or mixing, vibrating or agitating the solution or substrate, or both.

A preferred embodiment utilizes agitation or vibration. In general, the amount of manipulation of the substrate is minimized to prevent damage to the array; thus, preferred embodiments utilize the agitation of the solution rather than the array, although either will work. As will be appreciated by those in the art, this agitation can take on any number of forms, with a preferred embodiment utilizing microtiter plates comprising bead solutions being agitated using microtiter plate shakers.

The agitation proceeds for a period of time sufficient to load the array to a desired fill. Depending on the size and concentration of the beads and the size of the array, this time may range from about 1 second to days, with from about 1 minute to about 24 hours being preferred.

It should be noted that not all sites of an array may comprise a bead; that is, there may be some sites on the substrate surface which are empty. In addition, there may be some sites that contain more than one bead, although this is not preferred.

In some embodiments, for example when chemical attachment is done, it is possible to attach the beads in a non-random or ordered way. For example, using photoactivatible attachment linkers or photoactivatible adhesives or masks, selected sites on the array may be sequentially rendered suitable for attachment, such that defined populations of beads are laid down.

The arrays of the present invention are constructed such that information about the identity of the capture probe is built into the array, such that the random deposition of the beads in the fiber wells can be “decoded” to allow identification of the capture probe at all positions. This may be done in a variety of ways, and either before, during or after the use of the array to detect target molecules.

Thus, after the array is made, it is “decoded” in order to identify the location of one or more of the capture probes, i.e. each subpopulation of beads, on the substrate surface.

In a preferred embodiment, pyrosequencing techniques are used to decode the array, as is generally described in “Nucleic Acid Sequencing using Microsphere Arrays”, filed Oct. 22, 1999 (no U.S. Ser. No. received yet), hereby incorporated by reference.

In a preferred embodiment, a selective decoding system is used. In this case, only those microspheres exhibiting a change in the optical signal as a result of the binding of a target sequence are decoded. This is commonly done when the number of “hits”, i.e. the number of sites to decode, is generally low. That is, the array is first scanned under experimental conditions in the absence of the target sequences. The sample containing the target sequences is added, and only those locations exhibiting a change in the optical signal are decoded. For example, the beads at either the positive or negative signal locations may be either selectively tagged or released from the array (for example through the use of photocleavable linkers), and subsequently sorted or enriched in a fluorescence-activated cell sorter (FACS). That is, either all the negative beads are released, and then the positive beads are either released or analyzed in situ, or alternatively all the positives are released and analyzed. Alternatively, the labels may comprise halogenated aromatic compounds, and detection of the label is done using for example gas chromatography, chemical tags, isotopic tags mass spectral tags.

As will be appreciated by those in the art, this may also be done in systems where the array is not decoded; i.e. there need not ever be a correlation of bead composition with location. In this embodiment, the beads are loaded on the array, and the assay is run. The “positives”, i.e. those beads displaying a change in the optical signal as is more fully outlined below, are then “marked” to distinguish or separate them from the “negative” beads. This can be done in several ways, preferably using fiber optic arrays. In a preferred embodiment, each bead contains a fluorescent dye. After the assay and the identification of the “positives” or “active beads”, light is shown down either only the positive fibers or only the negative fibers, generally in the presence of a light-activated reagent (typically dissolved oxygen). In the former case, all the active beads are photobleached. Thus, upon non-selective release of all the beads with subsequent sorting, for example using a fluorescence activated cell sorter (FACS) machine, the non-fluorescent active beads can be sorted from the fluorescent negative beads. Alternatively, when light is shown down the negative fibers, all the negatives are non-fluorescent and the the postives are fluorescent, and sorting can proceed. The characterization of the attached capture probe may be done directly, for example using mass spectroscopy.

Alternatively, the identification may occur through the use of identifier moieties (“IMs”), which are similar to IBLs but need not necessarily bind to DBLs. That is, rather than elucidate the structure of the capture probe directly, the composition of the IMs may serve as the identifier. Thus, for example, a specific combination of IMs can serve to code the bead, and be used to identify the agent on the bead upon release from the bead followed by subsequent analysis, for example using a gas chromatograph or mass spectroscope.

Alternatively, rather than having each bead contain a fluorescent dye, each bead comprises a non-fluorescent precursor to a fluorescent dye. For example, using photocleavable protecting groups, such as certain ortho-nitrobenzyl groups, on a fluorescent molecule, photoactivation of the fluorochrome can be done. After the assay, light is shown down again either the “positive” or the “negative” fibers, to distinguish these populations. The illuminated precursors are then chemically converted to a fluorescent dye. All the beads are then released from the array, with sorting, to form populations of fluorescent and non-fluorescent beads (either the positives and the negatives or vice versa).

In an alternate preferred embodiment, the sites of attachment of the beads (for example the wells) include a photopolymerizable reagent, or the photopolymerizable agent is added to the assembled array. After the test assay is run, light is shown down again either the “positive” or the “negative” fibers, to distinguish these populations. As a result of the irradiation, either all the positives or all the negatives are polymerized and trapped or bound to the sites, while the other population of beads can be released from the array.

In a preferred embodiment, the location of every capture probe is determined using decoder binding ligands (DBLs). As outlined above, DBLs are binding ligands that will either bind to identifier binding ligands, if present, or to the capture probes themselves, preferably when the capture probe is a nucleic acid or protein.

In a preferred embodiment, as outlined above, the DBL binds to the IBL.

In a preferred embodiment, the capture probes are single-stranded nucleic acids and the DBL is a substantially complementary single-stranded nucleic acid that binds (hybridizes) to the capture probe, termed a decoder probe herein. A decoder probe that is substantially complementary to each candidate probe is made and used to decode the array. In this embodiment, the candidate probes and the decoder probes should be of sufficient length (and the decoding step run under suitable conditions) to allow specificity; i.e. each candidate probe binds to its corresponding decoder probe with sufficient specificity to allow the distinction of each candidate probe.

In a preferred embodiment, the DBLs are either directly or indirectly labeled. In a preferred embodiment, the DBL is directly labeled, that is, the DBL comprises a label. In an alternate embodiment, the DBL is indirectly labeled; that is, a labeling binding ligand (LBL) that will bind to the DBL is used. In this embodiment, the labeling binding ligand-DBL pair can be as described above for IBL-DBL pairs.

Accordingly, the identification of the location of the individual beads (or subpopulations of beads) is done using one or more decoding steps comprising a binding between the labeled DBL and either the IBL or the capture probe (i.e. a hybridization between the candidate probe and the decoder probe when the capture probe is a nucleic acid). After decoding, the DBLs can be removed and the array can be used; however, in some circumstances, for example when the DBL binds to an IBL and not to the capture probe, the removal of the DBL is not required (although it may be desirable in some circumstances). In addition, as outlined herein, decoding may be done either before the array is used to in an assay, during the assay, or after the assay.

In one embodiment, a single decoding step is done. In this embodiment, each DBL is labeled with a unique label, such that the the number of unique tags is equal to or greater than the number of capture probes (although in some cases, “reuse” of the unique labels can be done, as described herein; similarly, minor variants of candidate probes can share the same decoder, if the variants are encoded in another dimension, i.e. in the bead size or label). For each capture probe or IBL, a DBL is made that will specifically bind to it and contains a unique tag, for example one or more fluorochromes. Thus, the identity of each DBL, both its composition (i.e. its sequence when it is a nucleic acid) and its label, is known. Then, by adding the DBLs to the array containing the capture probes under conditions which allow the formation of complexes (termed hybridization complexes when the components are nucleic acids) between the DBLs and either the capture probes or the IBLs, the location of each DBL can be elucidated. This allows the identification of the location of each capture probe; the random array has been decoded. The DBLs can then be removed, if necessary, and the target sample applied.

In a preferred embodiment, the number of unique labels is less than the number of unique capture probes, and thus a sequential series of decoding steps are used. In this embodiment, decoder probes are divided into n sets for decoding. The number of sets corresponds to the number of unique tags. Each decoder probe is labeled in n separate reactions with n distinct tags. All the decoder probes share the same n tags. The decoder probes are pooled so that each pool contains only one of the n tag versions of each decoder, and no two decoder probes have the same sequence of tags across all the pools. The number of pools required for this to be true is determined by the number of decoder probes and the n. Hybridization of each pool to the array generates a signal at every address. The sequential hybridization of each pool in turn will generate a unique, sequence-specific code for each candidate probe. This identifies the candidate probe at each address in the array. For example, if four tags are used, then 4×n sequential hybridizations can ideally distinguish 4ⁿ sequences, although in some cases more steps may be required. After the hybridization of each pool, the hybrids are denatured and the decoder probes removed, so that the probes are rendered single-stranded for the next hybridization (although it is also possible to hybridize limiting amounts of target so that the available probe is not saturated. Sequential hybridizations can be carried out and analyzed by subtracting pre-existing signal from the previous hybridization).

An example is illustrative. Assuming an array of 16 probe nucleic acids (numbers 1-16), and four unique tags (four different fluors, for example; labels A-D). Decoder probes 1-16 are made that correspond to the probes on the beads. The first step is to label decoder probes 1-4 with tag A, decoder probes 5-8 with tag B, decoder probes 9-12 with tag C, and decoder probes 13-16 with tag D. The probes are mixed and the pool is contacted with the array containing the beads with the attached candidate probes. The location of each tag (and thus each decoder and candidate probe pair) is then determined. The first set of decoder probes are then removed. A second set is added, but this time, decoder probes 1, 5, 9 and 13 are labeled with tag A, decoder probes 2, 6, 10 and 14 are labeled with tag B, decoder probes 3, 7, 11 and 15 are labeled with tag C, and decoder probes 4, 8, 12 and 16 are labeled with tag D. Thus, those beads that contained tag A in both decoding steps contain candidate probe 1; tag A in the first decoding step and tag B in the second decoding step contain candidate probe 2; tag A in the first decoding step and tag C in the second step contain candidate probe 3; etc. In one embodiment, the decoder probes are labeled in situ; that is, they need not be labeled prior to the decoding reaction. In this embodiment, the incoming decoder probe is shorter than the candidate probe, creating a 5′ “overhang” on the decoding probe. The addition of labeled ddNTPs (each labeled with a unique tag) and a polymerase will allow the addition of the tags in a sequence specific manner, thus creating a sequence-specific pattern of signals. Similarly, other modifications can be done, including ligation, etc.

In addition, since the size of the array will be set by the number of unique decoding binding ligands, it is possible to “reuse” a set of unique DBLs to allow for a greater number of test sites. This may be done in several ways; for example, by using some subpopulations that comprise optical signatures. Similarly, the use of a positional coding scheme within an array; different sub-bundles may reuse the set of DBLs. Similarly, one embodiment utilizes bead size as a coding modality, thus allowing the reuse of the set of unique DBLs for each bead size. Alternatively, sequential partial loading of arrays with beads can also allow the reuse of DBLs. Furthermore, “code sharing” can occur as well.

In a preferred embodiment, the DBLs may be reused by having some subpopulations of beads comprise optical signatures. In a preferred embodiment, the optical signature is generally a mixture of reporter dyes, preferably flourescent. By varying both the composition of the mixture (i.e. the ratio of one dye to another) and the concentration of the dye (leading to differences in signal intensity), matrices of unique optical signatures may be generated. This may be done by covalently attaching the dyes to the surface of the beads, or alternatively, by entrapping the dye within the bead.

In a preferred embodiment, the encoding can be accomplished in a ratio of at least two dyes, although more encoding dimensions may be added in the size of the beads, for example. In addition, the labels are distinguishable from one another; thus two different labels may comprise different molecules (i.e. two different fluors) or, alternatively, one label at two different concentrations or intensity.

In a preferred embodiment, the dyes are covalently attached to the surface of the beads. This may be done as is generally outlined for the attachment of the capture probes, using functional groups on the surface of the beads. As will be appreciated by those in the art, these attachments are done to minimize the effect on the dye.

In a preferred embodiment, the dyes are non-covalently associated with the beads, generally by entrapping the dyes in the pores of the beads.

Additionally, encoding in the ratios of the two or more dyes, rather than single dye concentrations, is preferred since it provides insensitivity to the intensity of light used to interrogate the reporter dye's signature and detector sensitivity.

In a preferred embodiment, a spatial or positional coding system is done. In this embodiment, there are sub-bundles or subarrays (i.e. portions of the total array) that are utilized. By analogy with the telephone system, each subarray is an “area code”, that can have the same tags (i.e. telephone numbers) of other subarrays, that are separated by virtue of the location of the subarray. Thus, for example, the same unique tags can be reused from bundle to bundle. Thus, the use of 50 unique tags in combination with 100 different subarrays can form an array of 5000 different capture probes. In this embodiment, it becomes important to be able to identify one bundle from another; in general, this is done either manually or through the use of marker beads, i.e. beads containing unique tags for each subarray.

In alternative embodiments, additional encoding parameters can be added, such as microsphere size. For example, the use of different size beads may also allow the reuse of sets of DBLs; that is, it is possible to use microspheres of different sizes to expand the encoding dimensions of the microspheres. Optical fiber arrays can be fabricated containing pixels with different fiber diameters or cross-sections; alternatively, two or more fiber optic bundles, each with different cross-sections of the individual fibers, can be added together to form a larger bundle; or, fiber optic bundles with fiber of the same size cross-sections can be used, but just with different sized beads. With different diameters, the largest wells can be filled with the largest microspheres and then moving onto progressively smaller microspheres in the smaller wells until all size wells are then filled. In this manner, the same dye ratio could be used to encode microspheres of different sizes thereby expanding the number of different oligonucleotide sequences or chemical functionalities present in the array. Although outlined for fiber optic substrates, this as well as the other methods outlined herein can be used with other substrates and with other attachment modalities as well.

In a preferred embodiment, the coding and decoding is accomplished by sequential loading of the microspheres into the array. As outlined above for spatial coding, in this embodiment, the optical signatures can be “reused”. In this embodiment, the library of microspheres each comprising a different capture probe (or the subpopulations each comprise a different capture probe), is divided into a plurality of sublibraries; for example, depending on the size of the desired array and the number of unique tags, 10 sublibraries each comprising roughly 10% of the total library may be made, with each sublibrary comprising roughly the same unique tags. Then, the first sublibrary is added to the fiber optic bundle comprising the wells, and the location of each capture probe is determined, generally through the use of DBLs. The second sublibrary is then added, and the location of each capture probe is again determined. The signal in this case will comprise the signal from the “first” DBL and the “second” DBL; by comparing the two matrices the location of each bead in each sublibrary can be determined. Similarly, adding the third, fourth, etc. sublibraries sequentially will allow the array to be filled.

In a preferred embodiment, codes can be “shared” in several ways. In a first embodiment, a single code (i.e. IBL/DBL pair) can be assigned to two or more agents if the target sequences different sufficiently in their binding strengths. For example, two nucleic acid probes used in an mRNA a quantitation assay can share the same code if the ranges of their hybridization signal intensities do not overlap. This can occur, for example, when one of the target sequences is always present at a much higher concentration than the other. Alternatively, the two target sequences might always be present at a similar concentration, but differ in hybridization efficiency.

Alternatively, a single code can be assigned to multiple agents if the agents are functionally equivalent. For example, if a set of oligonucleotide probes are designed with the common purpose of detecting the presence of a particular gene, then the probes are functionally equivalent, even though they may differ in sequence. Similarly, an array of this type could be used to detect homologs of known genes. In this embodiment, each gene is represented by a heterologous set of probes, hybridizing to different regions of the gene (and therefore differing in sequence). The set of probes share a common code. If a homolog is present, it might hybridize to some but not all of the probes. The level of homology might be indicated by the fraction of probes hybridizing, as well as the average hybridization intensity. Similarly, multiple antibodies to the same protein could all share the same code.

In a preferred embodiment, decoding of self-assembled random arrays is done on the bases of pH titration. In this embodiment, in addition to capture probes, the beads comprise optical signatures, wherein the optical signatures are generated by the use of pH-responsive dyes (sometimes referred to herein as “ph dyes”) such as fluorophores. This embodiment is similar to that outlined in PCT US98/05025 and U.S. Ser. No. 09/151,877, both of which are expressly incorporated by reference, except that the dyes used in the present ivention exhibits changes in fluorescence intensity (or other properties) when the solution pH is adjusted from below the pKa to above the pKa (or vice versa). In a preferred embodiment, a set of pH dyes are used, each with a different pKa, preferably separated by at least 0.5 pH units. Preferred embodiments utilize a pH dye set of pka's of 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11, and 11.5. Each bead can contain any subset of the pH dyes, and in this way a unique code for the capture probe is generated. Thus, the decoding of an array is achieved by titrating the array from pH 1 to pH 13, and measuring the fluorescence signal from each bead as a function of solution pH.

Thus, the present invention provides array compositions comprising a substrate with a surface comprising discrete sites. A population of microspheres is distributed on the sites, and the population comprises at least a first and a second subpopulation. Each subpopulation comprises a capture probe, and, in addition, at least one optical dye with a given pKa. The pKas of the different optical dyes are different.

In a preferred embodiment, “random” decoding probes can be made. By sequential hybridizations or the use of multiple labels, as is outlined above, a unique hybridization pattern can be generated for each sensor element. This allows all the beads representing a given clone to be identified as belonging to the same group. In general, this is done by using random or partially degenerate decoding probes, that bind in a sequence-dependent but not highly sequence-specific manner. The process can be repeated a number of times, each time using a different labeling entity, to generate a different pattern of singals based on quasi-specific interactions. In this way, a unique optical signature is eventually built up for each sensor element. By applying pattern recognition or clustering algorithms to the optical signatures, the beads can be grouped into sets that share the same signature (i.e. carry the same probes).

In order to identify the actual sequence of the clone itself, additional procedures are required; for example, direct sequencing can be done, or an ordered array containing the clones, such as a spotted cDNA array, to generate a “key” that links a hybridization pattern to a specific clone.

Alternatively, clone arrays can be decoded using binary decoding with vector tags. For example, partially randomized oligos are cloned into a nucleic acid vector (e.g. plasmid, phage, etc.). Each oligonucleotide sequence consists of a subset of a limited set of sequences. For example, if the limites set comprises 10 sequences, each oligonucleotide may have some subset (or all of the 10) sequences. Thus each of the 10 sequences can be present or absent in the oligonucleotide. Therefore, there are 2¹⁰ or 1,024 possible combinations. The sequences may overlap, and minor variants can also be represented (e.g. A, C, T and G substitutions) to increase the number of possible combinations. A nucleic acid library is cloned into a vector containing the random code sequences. Alternatively, other methods such as PCR can be used to add the tags. In this way it is possible to use a small number of oligo decoding probes to decode an array of clones.

As will be appreciated by those in the art, the systems of the invention may take on a large number of different configurations, as is generally depicted in the Figures. In general, there are three types of systems that can be used: (1) “non-sandwich” systems (also referred to herein as “direct” detection) in which the target sequence itself is labeled with detectable labels (again, either because the primers comprise labels or due to the incorporation of labels into the newly synthesized strand); (2) systems in which label probes directly bind to the target analytes; and (3) systems in which label probes are indirectly bound to the target sequences, for example through the use of amplifier probes.

Detection of the reactions of the invention, including the direct detection of products and indirect detection utilizing label probes (i.e. sandwich assays), is preferably done by detecting assay complexes comprising detectable labels, which can be attached to the assay complex in a variety of ways.

In a preferred embodiment, an array of different and usually artificial capture probes are made; that is, the capture probes do not have complementarity to known target sequences. The adapter sequences can then be added to any target sequences, or soluble capture extender probes are made; this allows the manufacture of only one kind of array, with the user able to customize the array through the use of adapter sequences or capture extender probes. This then allows the generation of customized soluble probes, which as will be appreciated by those in the art is generally simpler and less costly.

When capture extender probes are used, in one embodiment, microsphere arrays containing a single type of capture probe are made; in this embodiment, the capture extender probes are added to the beads prior to loading on the array. The capture extender probes may be additionally fixed or crosslinked, as necessary.

Accordingly, the present invention provides compositions and methods for detecting the presence or absence of target analytes, including nucleic acid sequences, in a sample. As will be appreciated by those in the art, the sample solution may comprise any number of things, including, but not limited to, bodily fluids (including, but not limited to, blood, urine, serum, lymph, saliva, anal and vaginal secretions, perspiration and semen, of virtually any organism, with mammalian samples being preferred and human samples being particularly preferred); environmental samples (including, but not limited to, air, agricultural, water and soil samples); biological warfare agent samples; research samples (i.e. in the case of nucleic acids, the sample may be the products of an amplification reaction, including both target and signal amplification); purified samples, such as purified genomic DNA, RNA, proteins, etc.; raw samples (bacteria, virus, genomic DNA, etc.; As will be appreciated by those in the art, virtually any experimental manipulation may have been done on the sample.

The present invention provides compositions and methods for detecting the presence or absence of target nucleic acid sequences in a sample.

In a preferred embodiment, several levels of redundancy are built into the arrays of the invention. Building redundancy into an array gives several significant advantages, including the ability to make quantitative estimates of confidence about the data and signficant increases in sensitivity. Thus, preferred embodiments utilize array redundancy. As will be appreciated by those in the art, there are at least two types of redundancy that can be built into an array: the use of multiple identical sensor elements (termed herein “sensor redundancy”), and the use of multiple sensor elements directed to the same target analyte, but comprising different chemical functionalities (termed herein “target redundancy”). For example, for the detection of nucleic acids, sensor redundancy utilizes of a plurality of sensor elements such as beads comprising identical binding ligands such as probes. Target redundancy utilizes sensor elements with different probes to the same target: one probe may span the first 25 bases of the target, a second probe may span the second 25 bases of the target, etc. By building in either or both of these types of redundancy into an array, significant benefits are obtained. For example, a variety of statistical mathematical analyses may be done.

In addition, while this is generally described herein for bead arrays, as will be appreciated by those in the art, this techniques can be used for any type of arrays designed to detect target analytes. Furthermore, while these techniques are generally described for nucleic acid systems, these techniques are useful in the detection of other binding ligand/target analyte systems as well.

In a preferred embodiment, sensor redundancy is used. In this embodiment, a plurality of sensor elements, e.g. beads, comprising identical bioactive agents are used. That is, each subpopulation comprises a plurality of beads comprising identical bioactive agents (e.g. binding ligands). By using a number of identical sensor elements for a given array, the optical signal from each sensor element can be combined and any number of statistical analyses run, as outlined below. This can be done for a variety of reasons. For example, in time varying measurements, redundancy can significantly reduce the noise in the system. For non-time based measurements, redundancy can significantly increase the confidence of the data.

In a preferred embodiment, a plurality of identical sensor elements are used. As will be appreciated by those in the art, the number of identical sensor elements will vary with the application and use of the sensor array. In general, anywhere from 2 to thousands may be used, with from 2 to 100 being preferred, 2 to 50 being particularly preferred and from 5 to 20 being especially preferred. In general, preliminary results indicate that roughly 10 beads gives a sufficient advantage, although for some applications, more identical sensor elements can be used.

Once obtained, the optical response signals from a plurality of sensor beads within each bead subpopulation can be manipulated and analyzed in a wide variety of ways, including baseline adjustment, averaging, standard deviation analysis, distribution and cluster analysis, confidence interval analysis, mean testing, etc.

In a preferred embodiment, the first manipulation of the optical response signals is an optional baseline adjustment. In a typical procedure, the standardized optical responses are adjusted to start at a value of 0.0 by subtracting the integer 1.0 from all data points. Doing this allows the baseline-loop data to remain at zero even when summed together and the random response signal noise is canceled out. When the sample is a fluid, the fluid pulse-loop temporal region, however, frequently exhibits a characteristic change in response, either positive, negative or neutral, prior to the sample pulse and often requires a baseline adjustment to overcome noise associated with drift in the first few data points due to charge buildup in the CCD camera. If no drift is present, typically the baseline from the first data point for each bead sensor is subtracted from all the response data for the same bead. If drift is observed, the average baseline from the first ten data points for each bead sensor is substracted from the all the response data for the same bead. By applying this baseline adjustment, when multiple bead responses are added together they can be amplified while the baseline remains at zero. Since all beads respond at the same time to the sample (e.g. the sample pulse), they all see the pulse at the exact same time and there is no registering or adjusting needed for overlaying their responses. In addition, other types of baseline adjustment may be done, depending on the requirements and output of the system used.

Once the baseline has been adjusted, a number of possible statistical analyses may be run to generate known statistical parameters. Analyses based on redundancy are known and generally described in texts such as Freund and Walpole, Mathematical Statistics, Prentice Hall, Inc. New Jersey, 1980, hereby incorporated by reference in its entirety.

In a preferred embodiment, signal summing is done by simply adding the intensity values of all responses at each time point, generating a new temporal response comprised of the sum of all bead responses. These values can be baseline-adjusted or raw. As for all the analyses described herein, signal summing can be performed in real time or during post-data acquisition data reduction and analysis. In one embodiment, signal summing is performed with a commercial spreadsheet program (Excel, Microsoft, Redmond, Wash.) after optical response data is collected.

Methods for signal summing and analyses are included in U.S. Ser. No. 08/944,850, filed Oct. 6, 1997; 09/287,573, filed Apr. 6, 1999; and 60/238,866, filed Oct. 6, 2000; an PCT Nos. US98/21193, filed Oct. 6, 1998; and US00/09183, filed Apr. 6, 2000.

Once made, the methods and compositions of the invention find use in a number of applications. In a preferred embodiment, the compositions are used to probe a sample solution for the presence or absence of a target sequence, including the quantification of the amount of target sequence present. The compositions and methods find utility in the detection of genotyping assays and sequencing assays, and in all sorts of target analyte assays, including immunoassays.

For SNP analysis, the ratio of different labels at a particular location on the array indicates the homozygosity or heterozygosity of the target sample, assuming the same concentration of each readout probe is used. Thus, for example, assuming a first readout probe comprising a first base at the readout position with a first detectable label and a second readout probe comprising a second base at the readout position with a second detectable label, equal signals (roughly 1:1 (taking into account the different signal intensities of the different labels, different hybridization efficiencies, and other reasons)) of the first and second labels indicates a heterozygote. The absence of a signal from the first label (or a ratio of approximately 0:1) indicates a homozygote of the second detection base; the absence of a signal from the second label (or a ratio of approximately 1:0) indicates a homozygote for the first detection base. As is appreciated by those in the art, the actual ratios for any particular system are generally determined empirically.

Generally, a sample containing a target analyte (whether for detection of the target analyte or screening for binding partners of the target analyte) is added to the array, under conditions suitable for binding of the target analyte to at least one of the capture probes, i.e. generally physiological conditions. The presence or absence of the target analyte is then detected. As will be appreciated by those in the art, this may be done in a variety of ways, generally through the use of a change in an optical signal. This change can occur via many different mechanisms. A few examples include the binding of a dye-tagged analyte to the bead, the production of a dye species on or near the beads, the destruction of an existing dye species, a change in the optical signature upon analyte interaction with dye on bead, or any other optical interrogatable event.

In a preferred embodiment, the change in optical signal occurs as a result of the binding of a target analyte that is labeled, either directly or indirectly, with a detectable label, preferably an optical label such as a fluorochrome. Thus, for example, when a proteinaceous target analyte is used, it may be either directly labeled with a fluor, or indirectly, for example through the use of a labeled antibody. Similarly, nucleic acids are easily labeled with fluorochromes, for example during PCR amplification as is known in the art. Alternatively, upon binding of the target sequences, a hybridization indicator may be used as the label. Hybridization indicators preferentially associate with double stranded nucleic acid, usually reversibly. Hybridization indicators include intercalators and minor and/or major groove binding moieties. In a preferred embodiment, intercalators may be used; since intercalation generally only occurs in the presence of double stranded nucleic acid, only in the presence of target hybridization will the label light up. Thus, upon binding of the target analyte to a capture probe, there is a new optical signal generated at that site, which then may be detected.

Alternatively, in some cases, as discussed above, the target analyte such as an enzyme generates a species that is either directly or indirectly optical detectable.

Furthermore, in some embodiments, a change in the optical signature may be the basis of the optical signal. For example, the interaction of some chemical target analytes with some fluorescent dyes on the beads may alter the optical signature, thus generating a different optical signal.

As will be appreciated by those in the art, in some embodiments, the presence or absence of the target analyte may be done using changes in other optical or non-optical signals, including, but not limited to, surface enhanced Raman spectroscopy, surface plasmon resonance, radioactivity, etc.

The assays may be run under a variety of experimental conditions, as will be appreciated by those in the art. A variety of other reagents may be included in the screening assays. These include reagents like salts, neutral proteins, e.g. albumin, detergents, etc which may be used to facilitate optimal protein-protein binding and/or reduce non-specific or background interactions. Also reagents that otherwise improve the efficiency of the assay, such as protease inhibitors, nuclease inhibitors, anti-microbial agents, etc., may be used. The mixture of components may be added in any order that provides for the requisite binding. Various blocking and washing steps may be utilized as is known in the art.

The following examples serve to more fully describe the manner of using the above-described invention, as well as to set forth the best modes contemplated for carrying out various aspects of the invention. It is understood that these examples in no way serve to limit the true scope of this invention, but rather are presented for illustrative purposes. All references cited herein are incorporated by reference in their entirety.

### EXAMPLES

### Example 1

Immobilization of Crude Oligonucleotides to a Solid Support

1. Introduce chemical functional group (such as —NH2, —COOH, —NCO, —NHS, —SH, —CHO, etc.) onto solid support.

2. Activate the functional group before oligonucleotide attachment.

3. 5′-terminal modified oligonucleotide attachment.

Crude Oligonucleotides were attached to supports and compared to results from attachment of purified oligonucleotides. As demonstrated in FIG. 3, in the presence of 2 M salt, crude oligonucleotides were immobilized as efficiently as purified oligonucleotides.

IN addition, the improved attachment of oligonucleotides to a solid support in the presence of increased salt was sequence and length independent. Thus, the method finds use in attachment of all oligonucleotides to a solid support (see FIG. 4).

In addition, when 0.5 M to 3 M NaCl was used for attachment of oligonucleotides, non-purified oligonucleotides were attached with comparable efficiency when compared to purified oligonucleotides (see FIG. 5).

TABLE 1

| Seq. ID No. | Decoder (5′-3′) |
|---|---|
| 17 | GGCTGGTTCGGCCCGAAAGCTTAG |
| 18 | GTTCCCAGTGAAGCTGCGATCTGG |
| 19 | TACTTGGCATGGAATCCCTTACGC |
| 20 | ACTAGCATATTTCAGGGCACCGGC |
| 21 | GAACGGTCAATGAACCCGCTGTGA |
| 22 | GCGGCCTTGGTTCAATATGAATCG |
| 23 | GATCGTTAGAGGGACCTTGCCCGA |
| 24 | TGGACCTAGTCCGGCAGTGACGAA |
| 25 | ATAAACTACCCAGGACGGGCGGAA |
| 26 | CATCGGTTCGCGCCAATCCAGATA |
| 27 | GTCGGGCATAGAGCCGACCACCCT |
| 28 | CTTGGGTCATGATTCACCGTGCTA |
| 29 | TGCCTAACGTGCTAATCAGCAGCG |
| 30 | CGCATGTTGGAGCATATGCCCTGA |
| 31 | AGCCACTGCATCAGTGCTGTTCAA |
| 32 | GGTTGTTTTGAGGCGTCCCACACT |
| 33 | TCGACCAAGAGCAAGGGCGGACCA |
| 34 | GACATCGCTATTGCGCATGGATCA |
| 35 | GAAATACGAAGTCTGCGGGAGTCG |
| 36 | TGTCATGAATGATTGATCGCGCGA |
| 37 | ATATCGGGATTCGTTCCCGGTGAA |
| 38 | GCGAGCGTACCGAAGGGCCTAGAA |
| 39 | TTACCGGCAGCGGACTTCCGAATT |
| 40 | GTAATCGAGAGCTGCGCGCCGTCT |
| 41 | TCCCTGAGGTCGGAAGCTTCCGAC |
| 42 | CCTGTTAGCGTAGGCGAGTCGATC |
| 43 | TAGCGGACCGGCAGAATGAGTTCC |
| 44 | GGTACATGCACTACGCGCACTCGG |
| 45 | AATTCATCTCGGACTCCCGCGGTA |
| 46 | GCCAAATCTGGATTGGCAGGAATG |
| 47 | TGCATTTTCGGTTGAGGCACATCC |
| 48 | CCGCTCAATTCACCATGCTTCGCT |
| 49 | CTCGGAAAGGTGCAACTTTGGTGT |
| 50 | AATTCGACCAGCAGAACGTCCCAT |
| 51 | GCCAGAGTCTCAACCTCACGGGAT |
| 52 | CCAACAACTGGAACGGGAACCCGC |
| 53 | GAGAACTGATCGCTGAGGGGCATG |
| 54 | GGCACACTAGACTTGTGGCACCGA |
| 55 | CTTGGGCAAACGCTTCAGCCACAA |
| 56 | TCACATCCAAATATGGTCCGCGAA |
| 57 | GTCTGCCGGTGTGACCGCTTCATT |
| 58 | CATCGCAGAGCATAAACACCCTCA |
| 59 | GTTGGTATCTATGGCAGAGGCGGA |
| 60 | ACGAGGTGCCGCTGAGGTTCCATT |
| 61 | GGAATGAGTGGACCCAGGCACATT |
| 62 | TGTCAATATGCGTCCGTGTCGTCT |
| 63 | TGATGAGCCTCAGGGTACGAGGCA |
| 64 | CACCGCGGTGTTCCTACAGAATGA |
| 65 | TTGTTGCCAATGGTGTCCGCTCGG |
| 66 | TTAACCTGCGTCTGCCCCTTTCCT |
| 67 | AGGCGCGTTCCTGCCTTAGTGACG |
| 68 | TAGGGCGATGGCACGAAGCTTCAA |
| 69 | TGCATAGAGCCAAAGTCGGCGATG |
| 70 | TTGAGAGGCAGGTGGCCACACGGA |
| 71 | TCCGCATTGTGAGAAAAAACGAGC |
| 72 | GGCGGTTTCCGTAGCTATAGGTGC |
| 73 | GGTGAAAATTTCGTAGCCACGGGC |
| 74 | CCGACGGAGGATGAAGACAATCAC |
| 75 | CCAGTTTGGCCCAATTCGCCAAAA |
| 76 | GGATCTATTAGGCCGTGCGCACAG |
| 77 | CGGATGTCACCGTTTGGACTTTCA |
| 78 | ATCGCAAATCCTGCTCGTCCCTAA |
| 79 | CAGGGCATGCAATAATCGAGGTTC |
| 80 | CATGCGTTGATATATGGGCCCAAG |
| 81 | CAGCTGCAGCTTGTGACCAACCAC |
| 82 | TTGTATGTCTGCCGACCGGCGACC |
| 83 | GATGGCGCCCGTTGATAGGTATGG |
| 84 | ATGAGAATCGCCGGCAATCTGCTA |
| 85 | ATTTGCACTGACCGCAGGCTCGTG |
| 86 | CAGGGAGAACGGTTAAGTTCCCGT |
| 87 | AGGCCGGCGATCGAGGAGTTTGGT |
| 88 | ACACGGTGGTCTCTGATAGCGACC |
| 89 | GTGCAACGCCGAGGACTTCCATCA |
| 90 | TCGGTGCCTGATAGCCATTCCGAT |
| 91 | TGAAATACCACACAGCCAATTGGC |
| 92 | GCATCGTGTACATGACTGCCGCGA |
| 93 | CAGTGTTCTAACGGCGCGCGTGAA |
| 94 | CGCTTGCAACGTTGCACCTACTCT |
| 95 | CGAAAAACTAGTGGGCTCGCCGCG |
| 96 | CTTTCAGGGGAACTGCCGGAGTCG |
| 97 | TTGTGGCCTTCTTGTAAAGGCACG |
| 98 | TCCACGAACGGCGACCCGTTGTCT |
| 99 | CGACCTTGCACGAAACCTAACGAG |
| 100 | GTGCAGCTTCACGAGCCAGCCTGA |
| 101 | CGCTTTCGTGCGAATAGACGATGA |
| 102 | TGCGCTTACAGGCTCCTAGTGGTC |
| 103 | CACGCGCTTAGTCGCGATCGCATA |
| 104 | CGGAGGGAGGGAGCTAGCCTTCGA |
| 105 | GCATCCGGCCTGTTGATGACGCCT |
| 106 | AGGCCAATCGATCTTATTGCCGAG |
| 107 | CCTTCCAATGATTGCATACGccCA |
| 108 | AACACTTGATCAGGCGGGTCGTCT |
| 109 | TGGAATCAAGGCCGTAAAGGACAG |
| 110 | GCTCCCGTAACCTGTCCACCAGTG |
| 111 | AGTGGTGAATGGCCGCTACCCTGA |
| 112 | TGTTGAAGCGAGCTAAAACGGCCA |
| 113 | CAGCGCTCCAGAATTGACAGCAAT |
| 114 | AAGGTGGTGCCATTCATTTGGCTA |
| 115 | CGTTAAACCGCAATCCGTTCGGCT |
| 116 | TGTCTTCCACCTCGAAGGTTTCCA |
| 117 | CACGAGATACCGGCGTAAGGGTGG |
| 118 | CTACGGCAAACGTGTGGAATGGGT |
| 119 | GTAGGGCGATGACGGGCGAACTAC |
| 120 | AATCGACCTCCGCACACATTCGCA |
| 121 | GAGTCAGCATGGCGGCGGAGATTC |
| 122 | AGATAAAGACGCTGGCAACACGGG |
| 123 | GGTACCTCAACGCGAACCACTTGT |
| 124 | AAGCGATGGCTACCCAAGAGCGAT |
| 125 | AGAGCTTATGCAGAACCAGGCGCC |
| 126 | ATCGGTCTCACGCAGGGTTGGATA |
| 127 | TAGGTTGCCCGCCAGAAGAAACAT |
| 128 | CGGTGCTGTTGCAAAAGCCTGTAG |
| 129 | TGATGAAAGTTTGCGGCAGGACAC |
| 130 | GTTGAGTGCAGGATGCAGCGATAG |
| 131 | AACATTGCGCGGTCCACCAGGGTT |
| 132 | GGGCAGTTAGAGAGGGCCAGAAGT |
| 133 | TCGAGCTGGTCCCCGTGAACGTGT |
| 134 | GTCTTGGGGGCCGCTTAGTGAAAA |
| 135 | ACTGTTGGCTTGCTCTCATGTCCA |
| 136 | AGGACCATTCGGAAGGCGAAGATA |
| 137 | CTTGGGAGGCATCCGCTATAAGGA |
| 138 | AATAAACGGAACGCACCGCTACAG |
| 139 | TTGTACGTGCGGTCCCcATAAGCA |
| 140 | CGCACCAAACTGAGTTTCCCAGAC |
| 141 | ACCTGATCGTTCCCCTATTGGGAA |
| 142 | GGAACAGAGGCGAGGGGACTGAGC |
| 143 | CCCTGCCTTGGCGTGTCGGCTTAT |
| 144 | ACTCTGACACGCCAACTCCGGAAG |
| 145 | CTGACGGTTTTCATTCGGCGTGCC |
| 146 | TGCGGTGGTTCATTGGAGCTGGCC |
| 147 | GCATGGCCAACTAGTGACTCGCAA |
| 148 | AGGCCGTAAAGCGAATCTCACCTG |
| 149 | CGAATATTATGCCGAGAATCCGCG |
| 150 | ACAGACGAGCTCCCAACCACATGA |
| 151 | GGACGGTTTGTGCTGGATTGTCTG |
| 152 | AAAGGCTATTGAGTTGGTTGGGCG |
| 153 | GATGGCCTATTCGGAGATCGGGCC |
| 154 | GATCCAGTAGGCAGCTTCATCCCA |
| 155 | AATAACTCGCGCGGGTATGCTTCT |
| 156 | GGAGGAGGTTTGTCTCGGAAAGCA |
| 157 | CTTTGGTATGGCACATGCTGCCCG |
| 158 | AGAAAGGCTCGAGCAACGGGAACT |
| 159 | AATCTACCGCACTGGTCCGCAAGT |
| 160 | CGTGGCGGCCACAGTTTTTGGAGG |
| 161 | TTGCAGTTCAATCCATACGCACGT |
| 162 | GGCCCAAAGCCCCAGACCATTTTA |
| 163 | CGCCTGTCTTTGTCTCCGGACAAT |
| 164 | TGAGGCAACAGGGGCCAAAAACTA |
| 165 | AGCGGAAGTAGTCCTCGGCTCGTC |
| 166 | GGCCCCAAGGCTTAGAGATAGTGG |
| 167 | GCACGTGAAGTTTAACCGCGATTC |
| 168 | AGCGGCAGAAACGTTCCTTGACGG |
| 169 | TCGTCGAGCAGACGAGATTGCACG |
| 170 | TCTTTGCCGCGTAACTGACTGCTT |
| 171 | TTTATGTGCCAAGGGGTTAACCGA |
| 172 | TGTTACTGTGGTTCACGGCAGTCC |
| 173 | CGCGCCTCGCTAGACCTTTTATTG |
| 174 | ACAAATGCGTGAGAGCTCCCAACT |
| 175 | CGCGCAGATTATAGACCCGAATGT |
| 176 | CAAATAACGCCGCTGAATCGGCGT |
| 177 | CCTTCGTGCATCGGTGATGATGTT |
| 178 | TGAACACGAGCAACACTCCAACGC |
| 179 | CAGCAGATCCTTCGTAGCGGTCGT |
| 180 | GGAACCTGGTGAGTTGTGCCTCAT |
| 181 | TCATAAGCGACAATCGCGGGCTTA |
| 182 | CCCAACGTCACTGAAGCTCACAGT |
| 183 | TGTCAGAGCCCGCGACTCAGACGG |
| 184 | TACACGAAGCCTCTCCGTGGTCCA |
| 185 | CTCAGAAGTCCTCGGCGAACTGGG |
| 186 | ATCCTTTTATCTACTCCGCGGCGA |
| 187 | AGGCGTGCAGCAACAGGATAAACC |
| 188 | ACTCTCGAGGGAGTCTCTGGCACA |
| 189 | TTGCCAGGTCCATCGAGACCTGTT |
| 190 | TCCACTATAACTGCGGGTCCGTGT |
| 191 | GCCCAGTCGGCTCTAACAAGTTCG |
| 192 | CGGAACGGATAATCGGCGTCAGGT |
| 193 | TAAAATAAGCGCCTGGCGGGAGGA |
| 194 | GCGCACTCGTGAAACCTTTCTCGC |
| 195 | AGTTTGCCAGGTACTGGCAAGTGC |
| 196 | ACAACGAGGGATGTCCAGCGGCAT |
| 197 | TTCGCAGCACCCGCTAGGTACAGT |
| 198 | TAACCCGATTTTTGCGACTCTGCC |
| 199 | CGTCGCATTGCAAGCGTAGGCTTG |
| 200 | GAGCTGACGTCACCATCAGAGGAA |
| 201 | GGAGGCTGGGGGTCGCGCTTAAGT |
| 202 | TTGTGGGPACCGCACTAGCTGGCT |
| 203 | CCCTCGCACTGTGTTCACCCTCTT |
| 204 | TCATTGACTCGAATCCGCACAACG |
| 205 | ACAGGGGTTGGCCTTCGTACGTAC |
| 206 | AGGCCGTGCAACATCACACAGGAT |
| 207 | GGGCCGTGGTCACGTAATATTGGC |
| 208 | GCGCGGACATGAAACGACAAGGCC |
| 209 | CTTATTGGGTGCCGGTGTCGGATT |
| 210 | GGGGCGGTTACCAAAAAATCCGAT |
| 211 | GCTAAAGCGTGCTCCGTAACTGCC |
| 212 | ATCTCATGCATCTCGGTTCGTCGT |
| 213 | ACGAAAAAAGTGTGCGGATCCCCT |
| 214 | CCAAGTACACCGCACGCATGTTTA |
| 215 | ATCGTGCGTGGAGTGTCGCATCTA |
| 216 | TCCAGATACCGCCCCGPACTTTGA |
| 217 | TCTGCTGGCAGCACGTGAAGTGGC |
| 218 | TTGAAATTGCTCTGCCGTCAGTCA |
| 219 | AGTCAGGCGAGATGTTCAGGCAGC |
| 220 | ACAAGCCGACGTTAAGCCCGCCCA |
| 221 | CCCTAATGAGGCCAGTAACCTGCA |
| 222 | GTGAGACACACATCCCCTCCAATG |
| 223 | CGACGGATGCAGAGTTCAGTGGTC |
| 224 | CCCGCATGCCTGGCGGTATTACAA |
| 225 | TTAGCAAAGCGGCGCCGTTAGCAA |
| 226 | CCCGACACGGGTCAGCGTAATAAT |
| 227 | GCGACGGCCCTGAGGTATGTCGTC |
| 228 | CAAAAGTGTGTTCCCTTGCGCTTG |
| 229 | TCTCGAAGCACAGCCCGGTTATTG |
| 230 | ATGCTAACCGTTGGCCATGGAACT |
| 231 | CTTGCGGAGTGTTAGCCCAGCGGT |
| 232 | TGCTCCCTAGGCGCTCGGAGGAGT |
| 233 | CCAATGCCTTTGAGTAAGCGATGG |
| 234 | AGCAGATAACGTCCCAATGACGCC |
| 235 | TTGACCATTACGTGTTGCGCCCAT |
| 236 | TCGCGTATTTGCGGAATTCGTCTG |
| 237 | CTGCGTGTCAACAATGTCCCGCAG |
| 238 | TCTGGTGCCACGCAAGGTCCACAG |
| 239 | CTCCGGGAGGTCACTTAATTGCGG |
| 240 | TTTTCGTGATTGCCCGGAGGAGGC |
| 241 | TCGGGATGTAGCTGGGGCTACCGG |
| 242 | CGAGCCAACGCAAACACGTCCTTG |
| 243 | GCAAAGCCTTTGTGGGGCGGTAGT |
| 244 | ATTCGACCGGAAATGAGGTCTTCG |
| 245 | TTCGCTTGCTGAGTTGCTCTGTTC |
| 246 | CGCGTGAAGACCCCATTCCCGAGT |
| 247 | AACCGTATTCGCGGTCACTTGTGG |
| 248 | GGGGCCAACCGTTTCGAGGCGTAT |
| 249 | TTCGGCTGGCAGTCCAAACGGCTT |
| 250 | GGGTGTGGTTAGAATGCACGGTTC |
| 251 | GCGAGGACCGAACTAGACAAACGG |
| 252 | ACGCACGCGTGACCGAAGTTGCTG |
| 253 | TAAAAGGTCGCTTTGAAAGGGGGA |
| 254 | TGCGATCGCTAACTGCTGGGACAA |
| 255 | GGAGGTATAAGCGGAGCGGCCTCA |
| 256 | ATGCTGACATGTCGTGCACCTCGT |
| 257 | TGTGGTTAAAGCGTCCGTTCAACG |
| 258 | CGTTCACACCGGCGTAAGCTGCGT |
| 259 | CCTATCCCGGCGAGAACTTCTGTG |
| 260 | GTCTGCACTCACGCAGCGGAGGGA |
| 261 | GCACGAGTTGGTGCTCGGCAGATT |
| 262 | AACGTCGCACGACACACGTTCGTC |
| 263 | ATGCGCGCTTATCCTAGCATGGTC |
| 264 | TCACGTTTTCGTCTCGACATGAGG |
| 265 | TGTGCCTCATCCTTAGGATACGGC |
| 266 | AGGTGGTGTGGGTCAACCGCTTTA |
| 267 | CTGGATCGAAGGGACTGCAAGCTC |
| 268 | TAGATCAACTCGCGTACGCATGGA |
| 269 | GATCCTGCGGAGAAGAGAGTGCAG |
| 270 | TACGTGTGGAGATGCCCCGAACCG |
| 271 | GCGCTATGTCAATCGTGGGCGTAG |
| 272 | AGCGAGGTTTCTAGCGTCGACACC |
| 273 | CGATGAAGACAGGTTTGCTGTTGC |
| 274 | ACCCAGGTTTTGCCGTTGTGGAAT |
| 275 | CCCTGTTAACGGCTGCGTAGTCTC |
| 276 | AGGCCGATTTCACCCGCCAATTGC |
| 277 | GAGCCCTCACTCCTTGCCCTTTGA |
| 278 | GGGTGGACATCCGCCTCGCAGTCA |
| 279 | GATGGCTGAGAACCGTGCTACGAT |
| 280 | TCGACGTTAGGAGTGCTGCCAGAA |
| 281 | CGAATGGGTCTGGACCTTGCATAG |
| 282 | GTGCACCAGACATTCGAACTCGGA |
| 283 | AGAGGCCCCGTATATCCCATCCAT |
| 284 | AACGCCTGTTCAGAGCATCAGCGG |
| 285 | AAGGCTCAACACGCCTATGTGCGC |
| 286 | AGTCCGTGTTGCCAGATTGGCTCG |
| 287 | ATGTCCCATGTAAAGACGCGTGTG |
| 288 | ATGGAGTCTGCTCACGCCCAAAGG |
| 289 | CGGCCTCCAACAAGGAGCACTAAC |
| 290 | CAGAGCCGTGGCAACATTGCGAGC |
| 291 | TCATTTGAATGAGGTGCGCACCGG |
| 292 | GACGTACCGGAAGCGCCGTATAAA |
| 293 | ATGCGAGCAATGGGATCCGGATTC |
| 294 | AGAGTGAGGCCTCCCTGACCAGTG |
| 295 | CGCACCGTAAGTAGATTTGCCCGC |
| 296 | AGGGTATCGGAGCCAGGGCTTACC |
| 297 | TGAACCTTTGAGCACGTCGTGCGC |
| 298 | TCCGCCTTTTTGGTTACCTCGAAG |
| 299 | GAACGCCAACGGCACTAACACATC |
| 300 | CCGACAGCAGCCAAGACGTCCCAG |
| 301 | TTGTACACCTGGGCCACGCACAGG |
| 302 | CATAAAAAAACCTGGGGCTCTGCG |
| 303 | TGCCAACTGTGCAGACCGGACTTA |
| 304 | GGCGAAAGAGCGAAACCGGCTCGT |
| 305 | GGGATGCGTATTTTAGCGAACACG |
| 306 | TGGGATTCAGCGACCAGTACGCGA |
| 307 | CCCGATATTCGCCCGGCCTATTCG |
| 308 | CGAGAAGATGCCTCACGCAACCAA |
| 309 | AACCTTCACCCGTGGATGACGCTA |
| 310 | GGCTAGACGATGGATACCCGTGCC |
| 311 | GCCTCTTCTCGACGATGCGATTTT |
| 312 | GCTTCCGGATGAACGGGATGGTTG |
| 313 | CCCTCCATGTTCTTCGAACGGTTT |
| 314 | TTGATGGGCGGCAATGCTCTTGCT |
| 315 | ATTGTGAGATGCGCCAAATTCCCC |
| 316 | TCAGCACAGCCAGACGGTCAACTT |
| 317 | ACTCCACTCCTCGGTGGCAAACTA |
| 318 | TCTGGGCATGCCTGGACGGAGACG |
| 319 | TCTCAACTCCGGTACGACGAAACA |
| 320 | TTGCGTGGTCAAAGGCGCAACGTG |
| 321 | AGACAGCGATCCGCGGCTCATGAT |
| 322 | CGCGTCTCTAACTGAGAGCAGCCA |
| 323 | AGGCGCACATGTACGGACATTCAG |
| 324 | GATGAGTGGCACGTCGGTGTGTAA |
| 325 | TGATCCATATTGTCGGACGTTGCG |
| 326 | ACCTGCCGGGAGTTCATAGGCTAG |
| 327 | AGCATTGGCGTTTTTCCGCAACGA |
| 328 | GGTAATATTCAGCGCGACCGCTCA |
| 329 | ATAGCGTACGACGAGGTGACGCGC |
| 330 | GGGTGAGGGAAAGAGCACCTGCCT |
| 331 | TAGGTCACGATGCGTTTGACGCTA |
| 332 | ACTGCCCGTACCTCTGGTTCTGGC |
| 333 | CAAAAATCGGGTGAACATTGGCTG |
| 334 | CCTTTGGCCTGAAGTTGTCGTAGC |
| 335 | GTGCCCCACGAGCGTATCGTTGTA |
| 336 | AGGCGCTACGTGGGCCTGGAGCAA |
| 337 | GGGTGCTACCATTGCATTAGTCCG |
| 338 | ACCACGCGCGTACGTGTAACCGAG |
| 339 | CCATGATGCATTGGGTGCATTTAG |
| 340 | GGTCCGGCCCTACGAAACGTTCGA |
| 341 | CCGTGTGGCTGGAGATTCGTGTGA |
| 342 | GTTAGGGCGACGCATATTGGCACA |
| 343 | GGGTCAGTCAGGTGCGTTAGGATC |
| 344 | GCCGTGAAGTCGAATGCAGATCGA |
| 345 | GCCACCACCCAGTGCATTCAGGTA |
| 346 | GAGCTTAGTTTGCGGTCATCGGGC |
| 347 | TGTTTGCCGCCATTAGGGAGTAAC |
| 348 | GCTCCGCTGGATGTGCCGGTTTAG |
| 349 | CGGTAGCATGCGAGATCCCTGTTA |
| 350 | CTACGCTCTACCAGTTGCCTGCGA |
| 351 | GTGCCTCCTGCTGTATTTGCCAAG |
| 352 | TTGCGACTCGACTTGGACGAGTAG |
| 353 | TCTGGGAGCTGTTTACTCCAGCCA |
| 354 | TGCACGCGGAACTCCCTTTACCAT |
| 355 | TGGCAGCAAATGAATCGAAAGCAC |
| 356 | AACTGGTGACGCGGTACAGCGAAG |
| 357 | AGACGATTACGCTGGACGCCGTCG |
| 358 | ATGCCCTCCTTCATGGAAAGGGTT |
| 359 | ATTCTCGGAGCGTATGCGCCAGAA |
| 360 | ATAGCGGAGTTTGGGTACGCGAAC |
| 361 | ACCTACGCATACCGCTTGGCGAGG |
| 362 | GATTACCTGAATGGCCAAGCGAGC |
| 363 | CCTGTTAGCATCACGGCGCTTAGG |
| 364 | CGGAATGATGCGCTCGACAACGCT |
| 365 | TGAGAGAGGCGTTGGTTAAGGCAA |
| 366 | AAGCAGGCGAAGGGATACTCCTCG |
| 367 | TCACGACAGACGGGCCGAGATTAC |
| 368 | AAGCAATTTGGCCTCGTTTTGTGA |
| 369 | GCTGGTTGCGGTAGGATCGCATAT |
| 370 | TTGTGAATCCGTTCTGTCCCCGAC |
| 371 | CTCCGATGACAATTGTGGAGAGCA |
| 372 | TGGGCTCCTCTGAGGCGAGATGGC |
| 373 | GGATAGAGTGAATCGACCGGCAAC |
| 374 | TGCACCGAACGTGCACGAGTAATT |
| 375 | GCCAGTATTCTCGGGTGTTGGACG |
| 376 | TCGCTACCTAAGACCGGGCCATAC |
| 377 | TGGCATTGACGAGCAGCAGTCAGT |
| 378 | CGCGTCCCAGCGCCCTTGGAGTAT |
| 379 | ATGAAGCCTACCGGGCGACTTCGT |
| 380 | CCAGACAGATGGCCTGGAACCATG |
| 381 | TGGCGTGGGACCATCTCXAAGCTA |
| 382 | CCGCATGGGAACACGTGTCAAGGT |
| 383 | GCCCACTCGTCAGCTGGACGTAAT |
| 384 | ATTACGGTCGTGATCCAGAAAGCG |
| 385 | TGCGAGGTGAGCACCTACGAGAGA |
| 386 | GGGCCGCATTCTTGATGTCCATTC |
| 387 | CCTCGGATGTGGGCTCTCGCCTAG |
| 388 | TAGGCATGTTGGCGTGAGCGCTAT |
| 389 | CGATACGAACGAGGATGTCCGCCT |
| 390 | TACGCCGGTTAGCACGGTGCGCTA |
| 391 | CATACGATGTCCGGGCCGTGTCGC |
| 392 | ATCCGCAGTTGTATGGCGCGTTAT |
| 393 | GGGTAAGGGACAAAGATGGGATGG |
| 394 | ATTGGAGTGTTTTGGTGAATCCGC |
| 395 | GAACCGAGCCAACGTATGGACACG |
| 396 | GCCGTCAAGCTTAAGGTTTTGGGC |
| 397 | ACCTGCTTTTGGGTGGGTGATATG |
| 398 | AATCGTGGGCGCAGCAAACGTATA |
| 399 | GTCGCCGGATTGCTCAGTATAAGC |
| 400 | ACCCGTCGATGCTTCCTCCTCAGA |
| 401 | ATCCGGGTGGGCGATACAAGAGAT |
| 402 | TTCCGCATGAGTCAGCTTTGAAAA |
| 403 | GCAAAGTCCCACTGGCAAGCCGAT |
| 404 | CGACCTCGGCTTCATCGTACACAT |
| 405 | CTCATGAGCGCAGTTGTGCGTGAG |
| 406 | CAGATGAAGGATCCACGGCCGGAG |
| 407 | TCAAAGGCTCTTGGATACAGCCGT |
| 408 | TCCGCTAATTTCCAATCAGGGCTC |
| 409 | ACGCACGGCGCTTTTGCCTTAATG |
| 410 | TGACAACGTCACAAGGAGCAGGAC |
| 411 | CTTAGTTGGGGCGCGGTATCCAGA |
| 412 | GCTCTAATGCCGTGGAGTCGGAAC |
| 413 | CCGATTACAAATTGACTGACCGCA |
| 414 | AGACGTACGTGAGCCTCCCGTGTC |
| 415 | AATGGAGCGATACGATCCAACGCA |
| 416 | GGAGGCGCTGTACTGATAGGCGTA |
| 417 | TGTTTTTGAATTGACCACACGGGA |
| 418 | CATGTCTGGATGCGCTCAATGAAG |
| 419 | GCCCGCTAATCCGACACCCAGTTT |
| 420 | CCATTGACAGGAGAGCCATGAGCC |
| 421 | GAATCACCGAATCACCGACTCGTT |
| 422 | AACCAGCCGCAGTAGCTTACGTCG |
| 423 | TTTTCTGAGGGACACGCGGGCGTT |
| 424 | GGTGCTCCGTTTGATCGATCCTCC |
| 425 | CCGCTTAGGCCATACTCTGAGCCA |
| 426 | TAAGACATACCGACGCCCTTGCCT |
| 427 | GTTCCCGACGCCAGTCATTGAGAC |
| 428 | TAAAAGTTTCGCGGAGGTCGGGCT |
| 429 | CGGTCCAGACGAGCTGAGTTCGGC |
| 430 | CGGCGTAGCGGCTACGGACTTAAA |
| 431 | GCTTGGATGCCCATGCGGCAAGGT |
| 432 | AGCGGGATCCCAGAGTTTCGAAAA |
| 433 | GAGCTTGAGAGCGAGGTCATCCTC |
| 434 | GCATCGGCCGTTTTGACCATATTC |
| 435 | CATAGCGCTGCACGTTTCGACCGC |
| 436 | ACCCGACAACCACCAATTCAAAAA |
| 437 | GCGAACACTCATAAGAGCGCCCTG |
| 438 | TTTTGGTGTGGCCGGTTGAAGCTC |
| 439 | CCGCCGAGTGTAGAGAGACTCCGA |
| 440 | GACATCGGGAGCCGGAAACATGAG |
| 441 | TCGTGTAGACTCGGCGACAGGCGT |
| 442 | ATGCGCATATACTGACTGCGCAGG |
| 443 | ACAAGCGAACCCGAGTTTTGATGA |
| 444 | GCATGAGACTCCGCGAAGACATGT |
| 445 | TCCTACATGTCGCGTCACGATCAC |
| 446 | GACCGATCGCGAAGTCGTACACAT |
| 447 | GTCGCCAGGACTGGGCCGATGTGA |
| 448 | ACCGATAAGACTTGCATCCGAACG |
| 449 | TCCATAACCAGTCCGAAGTGCCGG |
| 450 | ACGCGCCCTGCATCTCGTATTTAA |
| 451 | AGACCGCATCAATTGGCGCGTACC |
| 452 | AGAGGCTTGGCAAGTAGGGACCCT |
| 453 | GCAATGGACGCCAGACGATACCGG |
| 454 | GCTGGACTTAGTCGTGTTCGGCGG |
| 455 | GGGGCTCATGAACGAAAGGCCTTT |
| 456 | AGGCATCGTGCCGGATTGCTCCCT |
| 457 | TGCGCATGTCGACGTTGAACAAAG |
| 458 | ATTGCATTATGCGGTCCCTCAAAC |
| 459 | TTCGGGTCACATCCGATGCCATAC |
| 460 | ACCCATCGCCGGAAAGCGATGTTG |
| 461 | AAGCGCTGACTCGGCTAAGAATCA |
| 462 | ACTTCCAAGTCCTTGACCGTCCGA |
| 463 | TCTCAATATTCCCGTAGTCGCCCA |
| 464 | AACAGTTCCTCTTTTTCCTGGCGC |
| 465 | CGTCCTCCATGTTGTCACGAACAG |
| 466 | TGCGCAGACCTACCTGTCTTTGCT |
| 467 | ATGGACGGCTTCGCAGTCCTCCTT |
| 468 | TGAACGCTTTCTATGGGCCACGTA |
| 469 | TGAACCCTGCCGCGAGCGATAACC |
| 470 | GTTCTTGCGCGATGAATCAGGACC |
| 471 | AGGGTACGTGTCGCAGCTTCGCGT |
| 472 | ACCCTTGCTCCGCCATGTCTCTCA |
| 473 | GGGACAAGGATTGAAGCTGGCGTC |
| 474 | TGTCGTTGCTCCCGAGTACCATTG |
| 475 | GTGGTTATCTGCGAGGGCTTTTGA |
| 476 | GTTGTCCGAGACGTTTGTGTCAGC |
| 477 | GCTGGTGAACACTCACGAACCGCT |
| 478 | GCAGACAGGGCAAATCGGTGCAAA |
| 479 | CCCATCACAACGAGTGGCGACTTT |
| 480 | GC1TCTACAGCTGGCGTGCTAGCG |
| 481 | GAATGTGTGCCGACCATTCTAGCC |
| 482 | CCAGCGGAAGTTAGAGCTCTGTGG |
| 483 | TTTTTACCGACCACTCCATGTCGG |
| 484 | GCGGCTATGTGATGACGGCCTAGC |
| 485 | AGTACACGGGCGTGTTAGCGCTCC |
| 486 | TCCTGTGTGGTGGCGCACTCCCAC |
| 487 | CCAACTAACCAATCGCGCGGATGA |
| 488 | AGTGAGTGACCAAGGCAGGAGCAA |
| 489 | CATCTTTCGCGGAGTTTATTGCGG |
| 490 | CTTCGTCCGGTTAGTGCGACAGCA |
| 491 | CTCACGAAAACGTGGGCCCGAAAT |
| 492 | CGCAGCAGCTGAACTCTAGCATTG |
| 493 | AGGAGACATACGCCCAAATGGTGC |
| 494 | ATTGAGAACTCGTGCGGGAGTTTG |
| 495 | CTCTTTGTAGGCCCAGGAGGAGCA |
| 496 | GCCGCAGGGTCGATAATTGGTCTA |
| 497 | AAACGCCGCCCTGAGACTATTGGG |
| 498 | CTGAGTTGCCTGGAACGTTGGACT |
| 499 | CGGATGGGTTGCAGAGTATGGGAT |
| 500 | CTGACCTTTGGGGGTTAGTGCGGT |
| 501 | GGAAATGAGAACCTTACCCCAGCG |
| 502 | AACGCATCGTCCGTCAACTCATCA |
| 503 | TGGAGAGAGACTTCGGCCATTGTT |
| 504 | ACGGAAGTCACGGCGTCGCTCGAA |
| 505 | TTGCGCTCATTGGATCTTGTCAGG |
| 506 | AGCGCGTTAAAGCACGGCAACATT |
| 507 | AGCCAGTAAACTGTGGGCGGCTGT |
| 508 | CGACTGATGTGCAACCAGCAGCTG |
| 509 | GGTTGCTCATACGACGAGCGAGTG |
| 510 | GCGCAAATCCACGGAACCCGTACC |
| 511 | ACGCAGTTTATTCCCCTGGCTTCT |
| 512 | AGAACCTCCGCGCCTCCGTAGTAG |
| 513 | AAAGGAGCTTTCGCCCAACGTACC |
| 514 | AGTGATTGTGCCACTCCACAGCTC |
| 515 | GCGATCGTCGAGGGTTGAGCTGAA |
| 516 | GGGAGACAGCCATTATGGTCCTCG |
| 517 | GAGACGCTGTCACTCCGGCAGAAC |
| 518 | CCACCGGTCGCTTAAGATGCACTT |
| 519 | CGGCATAACGTCCAGTCCTGGGAC |
| 520 | AAGCGGAACGGGTTATACCGAGGT |
| 521 | TGCACACTAGGTCCGTCGCTTGAT |
| 522 | AGGGAACCGCGTTCAAACTCAGTT |
| 523 | GAATTACAACCACCCGCTCGTGTT |
| 524 | TTCAGTGCTCACGAAGCATGGATT |
| 525 | TTAGTTTGGCGTTGGGACTTCACC |
| 526 | AATGCGACCTCGACGAGCCTCATA |
| 527 | CCGAAACCGTTAACGTGGCGCACA |
| 528 | TAAAGTAACAAGGCGACCTCCCGC |
| 529 | TAATGATTTTAGTCGCGGGGTGGG |
| 530 | GGCTACTCTAAGTGCCCGCTCAGG |
| 531 | TGGCGGACGACTCAATATCTCACG |
| 532 | GGGCGTTAGGCGTAATAGACCGTC |
| 533 | GCCACCTTTAGACGGCGGCTCTAG |
| 534 | GAGATGTGTAAACGTGCAGGCACC |
| 535 | CAACCTCGTTGTCGAGTTTCTCGG |
| 536 | TAGCTCGTGGCCCTCCAAGCGTGT |
| 537 | GTGTCGGCGCTATTTGGCCTTACC |
| 538 | CCAGGGAAGCAACTGGTTGCCATT |
| 539 | TTCCGAAACTAAGCCAGAACCGCT |
| 540 | GCAAACCCGGTAACCCGAGAGTTC |
| 541 | GCAAATGGCGTCATGCACGAACGT |
| 542 | AGTACTTTCGCGCCCAGTTTAGGG |
| 543 | AAGATCTGCGAGGCATCCCGGCTT |
| 544 | GCAAGTGTATCGCACAGTGCGATT |
| 545 | CCGACAAGGCCTCAATTCATTCTG |
| 546 | GTCTCGTCTCAACTTTAAGGCGCG |
| 547 | ATCCAGAGATCCGTTTTGCAGCGT |
| 548 | GTCACCAGGAGGGAAGTTTCACCC |
| 549 | TATCTTACGCCCCACGGTCGAGCT |
| 550 | TTCCGTCAGGCGGATCAACGGAAT |
| 551 | ATGCCGGACACGCATTACACAGGC |
| 552 | TGGGCCGCTTGGCGCTTTCATAGA |
| 553 | CCTAGCGCGAGCTTTACTGACCAG |
| 554 | TTGGCCAGGAATATGGTCTCGAGA |
| 555 | GTCTGCGGCCGACTTGCTATGCAT |
| 556 | AACTTGCTCATTCTCAAGCCGACG |
| 557 | ACGTCAGCGATTGTGGCGAAATAT |
| 558 | ACGGCCTGCGTCAGCACATGCATC |
| 559 | ATACCTCCGCAGAACCATTCCGTT |
| 560 | AGTTCGCGGTCCCACGATTCACTT |
| 561 | TGCTCAATTTGTGCAGAAAACGCC |
| 562 | TTATCGCGAGAGACGACCGTGTCC |
| 563 | GACGCGACGTGAGTAGTGGAAGCG |
| 564 | ATGGTAGGGGCATTGGGCTTTCCT |
| 565 | CCAAATATAGCCGCGCGGAGACAT |
| 566 | GCAAACCCTGATTGAATCGTGCCC |
| 567 | TAGCGTCTTGCGTGAAACCATGGG |
| 568 | CCACCCCGACAGCGCTGGACTCTT |
| 569 | ACGAGCACTGAAGGCTGCTTTACG |
| 570 | CATATCAGCGTCGTCTAGCTCGCG |
| 571 | TGATCCCGGACCGGCTAGACTAAT |
| 572 | GGCCCCGACACTACAGGGTAATCA |
| 573 | GGCTCCAGGGCGAGATTATGAATG |
| 574 | CAAAATCCGATGGGCGGAAAATTA |
| 575 | CACAGGCGCATAGGGAGCAAGCTA |
| 576 | TAGCTATTGCCCCGATGGGCTACT |
| 577 | TGGTACGCGGTCCATAGCAAGTCG |
| 578 | GACGCTGTGGCTCGGAAACTGTTC |
| 579 | CCTGGGTTCGCCGCGTGGTAACTG |
| 580 | TTCCCGCGTAGCCCAACAGCTATA |
| 581 | TTCGCGGATTGCTGOCGCATAACA |
| 582 | AAAAATGGCACCGAAGTTGAGGCA |
| 583 | CATTCCGCGCGAGTTGAAATCCAG |
| 584 | ACGCACGTTTTTTGGGACGGTTAA |
| 585 | TGTCCATGACGTCGTTTCTCTGGT |
| 586 | TCTCAGTCGGACTCGTATGCCAGA |
| 587 | CTCCAAACGCACACATCAAGCATC |
| 588 | TTCAACCAAGCGGGGTGTTCGTGA |
| 589 | GGTGTCGGAGGGTGGTGACCTCGA |
| 590 | AGCGCTTTTGGTCATGATTTGCAA |
| 591 | CCGAGGACTTACGTCTGCCCAGGA |
| 592 | GCCCAATCCAGTTCTTATGCGCCC |
| 593 | AAGCTTTGCGAAAGGTGTGTTGGC |
| 594 | CGGGTTAACCCACGCAAGTTATGA |
| 595 | TGATTAGCGCTCAATACACGCGTG |
| 596 | AAGGGCAGACCTTTGGTTCGACTG |
| 597 | GCGCCACAAGATTCACATGTCATT |
| 598 | GCCATGTTCAAGGGCCTTTCGAAG |
| 599 | CGCGGTGTTTTGTCTAGGTGCCGG |
| 600 | CAACATTGTGGTGGCACTCCATCC |
| 601 | CGATACGCGCCGGTTTGTTAAATC |
| 602 | GGCTATAAACGTGCGGACTGCTCC |
| 603 | TGGGTAAATCACTATTGCGCGGTT |
| 604 | GTCTTCATCGGCCCGCGCAAGCTA |
| 605 | GCGACACACCCTGTACTCTGATGC |
| 606 | GTAGCAGGGTCCGCAAGACCAAGC |
| 607 | TCGCCAACGCAGGGTAACTGCCAT |
| 608 | ACTCCGAAGCTTCGAGCGGCACGA |
| 609 | TCCCGCCCACTAGACTGACTCGTA |
| 610 | ACCTTCTGGGGTCGCTCACCAATA |
| 611 | ATCATCCCACGGCAGAGTGAAGAG |
| 612 | CGCTGGACTGGCCTATCCGAGTCG |
| 613 | CGGTCTCAGCAACACTGTCGCAAA |
| 614 | CGAACGTTCTCCGATGTAATGGCC |
| 615 | ATACCGTGCGACAAGCCCCTCTGA |
| 616 | AGCTCATTCCCGAGACGGAACACC |
| 617 | TTTCATGCGGCCGTTGCAAATCAT |
| 618 | ACTCGAACGGACGTTCAATTCCCA |
| 619 | CTGCATGGTGTGGGTGAGACTCCC |
| 620 | CCGCGAGTGTGGATGGCGTGTTGA |
| 621 | AATGTGTCGGTCCTAAGCCGGGTG |
| 622 | TAAGACGAGCCTGCACAGCTTGCG |
| 623 | GGCGTGGGAGGATAAGACGATGTC |
| 624 | TGCTCCATGTTAGGAACGCACCAC |
| 625 | CGGTGTTGGTCGGACTGACGACTG |
| 626 | CCGCGCGTATCTATCAGATCTGGG |
| 627 | AAAGCATGCTCCACCTGGAGCGAG |
| 628 | ACTTGCATCGCTGGGTAGATCCGG |
| 629 | TGCTTACGCAGTGGATTGGTCAGA |
| 630 | ATGCAGATGAACAAATCGCCGAAT |
| 631 | GCAATTCTGGGCCATGTATTCGTC |
| 632 | AGGGTTCCTTACGCGTCGACATGG |
| 633 | GTGGAGCTAATCGCGAGCCTCAGA |
| 634 | TCGTAGTCTCACCGGCAATGATCC |
| 635 | TTATAGCAGTGCGCCAATGCTTCG |
| 636 | CGAACAGTGCTGTCCGTCGCTCAA |
| 637 | TCCGCGTGGACTGTTAGACGCTAT |
| 638 | CATTAGCCCGCTGTCGGTAACTGT |
| 639 | GGAAAGAAACTCAGACGCGCAATG |
| 640 | CGACTCGCTGGACAGGAGAATCGT |
| 641 | CATGATCCTCTGTTTCACCCCCGG |
| 642 | GGCGTAGCGCTCTAAAAGCTTCGG |
| 643 | AGTGATGCCATCAGGCCCGTATAC |
| 644 | TATGGAAAGGGCAACAGCGCTATC |
| 645 | CTGTGGTTGATGGAGGATCCACAC |
| 646 | ACTCGCTGGAATTTGCGCTGACAC |
| 647 | CAGGCCCGAACCACGCGGTTACAG |
| 648 | GGCGCAATGGGCGCATAAATACTA |
| 649 | GGTCAATTCGCGCTACATGCCCTA |
| 650 | TGAGGGCTGTTTGGTATTTGACCC |
| 651 | GATGGTGGACTGGAGCCCTTCCGC |
| 652 | CCGCGCATAGCGCAATAGGGGAGA |
| 653 | TCTTCTGGCTGTCCGGCACCCGAA |
| 654 | GCGTTCGCAATTCACGGGCCCTTA |
| 655 | TCGTTTCGGCCTTGGAGAGTATCG |
| 656 | AGGTGCAAGTGCAAGGCGAGAGGC |
| 657 | CGCCAGTTTCGATGGCTGACGTTT |
| 658 | GCTTTACCGCCGATCCCAGATATC |
| 659 | GTGCTTGACGAAGAGGCGAAATGT |
| 660 | CAGTCCGTGCGCTTCATGTCCTCA |
| 661 | TACGCGTAAGAGCCTACCCTCGCG |
| 662 | GGCGAGTCTTGTGGGGACATGTGT |
| 663 | CCAAAGCGAAGCGAGCGTGTCTAT |
| 664 | GCCGTAGGTTGCTCTTCACCGAAC |
| 665 | AAATCCGCGATGTGCCGTGAGGCT |
| 666 | GGCTTCGCACCCGTACCAATTTAG |
| 667 | TGTAGAGTCCCACGTAGCCGGCAT |
| 668 | CACTAGTCTGGGGCAAGGTGCATT |
| 669 | TGTACTCGGCAGGCGCAATAGATT |
| 670 | AACGGGTATCGGAAGCGTAAAAGC |
| 671 | CGGACTGCCCGTTTGCAAGTTGAG |
| 672 | ATCGTTCAGCACTGGAGCCCGTAA |
| 673 | ATGCATCGAACTAGTCGTGACGGC |
| 674 | TTCCAGGCATTAAGGAGAGGGAGC |
| 675 | GTGCGACATCTACTCCACGATCCC |
| 676 | CTCATCGTCCTAACACGAGAGCCC |
| 677 | AATGGCACTTCGGCGGTGATGCAA |
| 678 | CCGTGGGAGGGAATCCAACCGAGG |
| 679 | AAATTCTCGTTGGTGACGGCTCAT |
| 680 | TTGCTCTTATCCTTGTCCTGGGCG |
| 681 | TTAAGGATCAGGCGGAGCTTGCAG |
| 682 | CGCGACTAAGGTGCTGCAACTCGA |
| 683 | GCTCGATTTCACGGCCCGTTGTTC |
| 684 | AGCAGAGTGCGTTGCAGAGGCTAA |
| 685 | TGGAGGTGAGGACGACGTGCACTA |
| 686 | AACCGTTTAGGGTACATTCGCGGT |
| 687 | TATGATCGCTCGGCTCACAGTTTG |
| 688 | GACTTTTTGCGGAAACGTCATGGT |
| 689 | TGTCGGTTATTCCACCTGCAAGGA |
| 690 | CTATGGTTTGCACTGCGCCGTCGA |
| 691 | AGCAGGGAAATTCAATCGTTCGCA |
| 692 | CCTAACCGAGCGCTTAGCATTTCC |
| 693 | CCCGACCCTAACTCGCATTGAATA |
| 694 | TTGCTTAATGGTGACGCCACGGAT |
| 695 | GATGCTCGCCGTGTTTAGTTCACG |
| 696 | TCGGATGACGAGTTTCCATGACGG |
| 697 | ATGCGGTCTACTTTCTCGATCGGG |
| 698 | TTGCGAGGCTAAGCACACGGTAAA |
| 699 | AACTTAATTACCGCCTCTGGCGCC |
| 700 | GTGACCGCGAACTTGTTCCGACAG |
| 701 | TGCGGATTACCGATTCGCTCTTAA |
| 702 | TGATAGGGGGCCACGTTGATCAGA |
| 703 | TCGCTCCGTAGCGATTCATCGTAG |
| 704 | TGTCAGCTGGTAGCCTCCGTTTGA |
| 705 | AGCGTCGCATGACGCTTACGGCAC |
| 706 | TCACTCAGCGCTGTGACTGCCTGA |
| 707 | GTTTGCGCTATAGTGGGGGACCGT |
| 708 | GTCGCATTCTGCACTGGCTTCGCC |
| 709 | TGATTAGGTGCGGTCCCGTAGTCC |
| 710 | AAGGGACCTTGGGTGACGGCGAGA |
| 711 | TCAAATGGCCACCGCGTGTCATTC |
| 712 | CTCCGACGACCAATAAATAGCCGC |
| 713 | GGCTATTCCCGTAGAGAGCGTCCA |
| 714 | TGGATAACCTCTCGGTCCATCCAC |
| 715 | GACCGCTGTACGGGAGTGTGCCTT |
| 716 | GCCACAGAGTTTTAGCAGGGACCC |
| 717 | CCCACGCTTTCCGACCACTGACCT |
| 718 | CATTGACACAATGCGGGGACTGAT |
| 719 | AGCCACTCGACAGGGTTCCAAAGC |
| 720 | CAGGATGAGCAAAGCGACTCTCCA |
| 721 | CAAGGTATGGTCTGGGGCCTAAGC |
| 722 | GGTGTTCGGCCTAAACTCTTTCGG |
| 723 | TTTAGTCGGACCCTGTGGCAATTC |
| 724 | CACACGTTTCCGACCAGCCTGAAC |
| 725 | CTGGACGAACTGGCTTCCTCGTAC |
| 726 | TTCACAATCCGCCGAAAACTGACC |
| 727 | AACAGGATATCCGCGATCACGACA |
| 728 | TACGTCGGATCCATTGCGCCGAGT |
| 729 | CATGGATCTCTCGGTTTGATCGCC |
| 730 | AGCCAGGCGCGTATATACGCTCGG |
| 731 | ATTTGGCACGTGTCGTGCCATGTT |
| 732 | CCGCGTTGCACCACTTTGAGGTGC |
| 733 | TTGGACGTGACAAGCATGGCGCTC |
| 734 | CTGAATCGCGCAAGTAAATGGGGG |
| 735 | GATAAGGTCCACCAGATTGCGCGC |
| 736 | CTAACAATTGCCAACCGGGACGGC |
| 737 | GGTAACCTGGGTGCTTGCAGGTTA |
| 738 | ATCGGAGCCACCATTCGCATTGGG |
| 739 | GTGAACTGGCTTGCCCCAGGATTA |
| 740 | AGGCGATAGCATGGTCCCATATGA |
| 741 | AACGGTATCGTGGCTAATGCACGA |
| 742 | AGTAGTGGTCCTCCAGATCGGCAA |
| 743 | CCGTTGAATTGGACGGGAGGTTAG |
| 744 | GCATAAGTGCGGCATCGCGAAGGG |
| 745 | CGACAAGATGCAGCTGCTACATGC |
| 746 | TCGCAGTGATTCCCGACCGATAAG |
| 747 | CAAGGCGAGTCCACTCGAGGGGAC |
| 748 | GCAACTTGCACGGCATAAGTGGCC |
| 749 | TCCGAGCTTGACGTTCGCGACGTC |
| 750 | AGCGCTGGGCTGTGCTGCCATCTC |
| 751 | TTCATGTCGCTGAGTAACCCTCGC |
| 752 | CGAACCGCTAATGCCCATTGTCAG |
| 753 | CACGGAAGGTGGGACAAATCGCCG |
| 754 | CACAGATGGAGACAAACGCGCCTT |
| 755 | TTTTCGCAACTCGCTCCATAACCC |
| 756 | ACGTTACGTTTCCGGCGCCTCTAA |
| 757 | TATCGGATTGCGTGGGTTTCAATC |
| 758 | CTTCCACAATTGTCTGCGACGCAC |
| 759 | TGCACAAAGGTATGGCTGTCCGGC |
| 760 | ACCGTGGCCGGGCCATAAGCTACG |
| 761 | TCCGATGCCAGTCCCATCTTAAGA |
| 762 | CTGAAACCGTGCGAATCGAGGTGA |
| 763 | CGGTGTTCCGCGTGTCGAAAAAAT |
| 764 | TCTAGCAGGCCTTTTGAATCGCCA |
| 765 | GAGTCACCTCTGAGACGGACGCCA |
| 766 | TCTTCTGTCATCCTGCAGCAGCAT |
| 767 | GCGGATGAAACCTGAAAGGGGCCT |
| 768 | GGGGCCCCAAACTGGTATCAAGCC |
| 769 | GCATTGGCTTCGGATTCTCCTACA |
| 770 | AGGCGGCCCAACTGTGAGGTCTTG |
| 771 | ACACCATGTGCTCCGCGCTGCAGT |
| 772 | ACGATGAACATGAATCGGGAGTCG |
| 773 | CTGCATCCCTGTAGCAGCGCTCCG |
| 774 | GTGCCGTATTTCGACCTGTGCGTT |
| 775 | GCAGTGCGCACTTCAGTTCAAAAG |
| 776 | GCGATTTTAAGCGATGCCTTGACG |
| 777 | TAGGTGACCTAGGCTTGCTTGCGG |
| 778 | CTGGATACCTTGCCTGTGCGGCGC |
| 779 | CCCCTTACGGCTCGTCGTCTATGC |
| 780 | GCGCTTGCCCGATGCGATGCATTA |
| 781 | TTTCTGTAAGCGGCCTGGGGTTCA |
| 782 | GGCTGAGGTGAGCGGTAAGGATGA |
| 783 | TCTTGGCCTCCCCGATCTAATTTG |
| 784 | GGAGGTAACGCCGTGTACGTAGGA |
| 785 | GTAATCCATTTGTGGCTGCGTCAA |
| 786 | CAAACCCATTCCAGCAGACGCCTG |
| 787 | TAGGAGGAATTTGGCATGCGGGCG |
| 788 | ATAGGTAGGATGTGCCCGGCGTTG |
| 789 | GCAAGTGCTTAGCTCGTCAGCCTC |
| 790 | CTGGCTGTGTCGCATCTCGTTAAC |
| 791 | CTAACGTCGTCTCGCGCAATCACT |
| 792 | TTTTCATAAACGTTGTCCCCGAGC |
| 793 | AGCAGGAGGACGAACCTCCGCTCC |
| 794 | TTCAAGCACCATCGTGCAATCCAA |
| 795 | AGCGTCGCCAGTGATCGCTAGTGG |
| 796 | TACATTCCCTGCCTCCGTGGGCTT |
| 797 | CGCTTCGCGTATTCAGTAGCGGTT |
| 798 | TCGGACGCGTCGACACTCATTATA |
| 799 | TCTGAGCAGGCCAGCGCTCCAGCT |
| 800 | TTGAATTGCCAAGCCCTGAAAGCC |
| 801 | AGTTTTCGCCTTGATGCGTCGGTG |
| 802 | GTTTCATAGGCCACGCGTGCTAAA |
| 803 | GGAGCGAAGACTTCGTCTGCCCAA |
| 804 | ATTGGCCGAGGGTGAATGCAGCCT |
| 805 | TGATCCATCCGAATGCTTTTCCAT |
| 806 | GCACACAGTTGTCTTGGCCCATGA |
| 807 | CTGGCGGGCAGTGGAAAAAACAAC |
| 808 | ATCTCCATGCGTAAGACTGCTCCG |
| 809 | TCTCCTCTCGTCGCAGTTCGTGGA |
| 810 | TAGCGTATTCACTCTTGCCGAGCA |
| 811 | CAATCAAAAGCCACGGCGCGATGG |
| 812 | AGCGTCACGGAATTCAGCAGATCT |
| 813 | GACTCCCTGTTAATGCGCCCAAGG |
| 814 | TAGGCACTGCCGGTTCAGATTCAA |
| 815 | AACAGGGTGATAACGGTGGCCAAT |
| 816 | CGTGCGTACCATGTGTAAGTGCGT |
| 817 | GACCAATTCTACTTCGGCAGCCCA |
| 818 | ATCGGACCGATTTGCTTTTGGCTG |
| 819 | TCCGCCGAAGCACACGCTTATTCG |
| 820 | AACGGTACGCATTGTGAGCAGTGT |
| 821 | TGGCGACTACTGTTCCCCTGAATC |
| 822 | CAGAGGGGACAGCCGTATGCCTTA |
| 823 | CGGTGGTTTTATCGGAATCTGCGA |
| 824 | TTGGCCTCCGACCTCACGACATAT |
| 825 | CGTTTCGCTAGCATCTGGCGCCGA |
| 826 | ACTAAGCGGTGGAGCCGGTGGATG |
| 827 | ATATTGGCTGCGTTTACGGGCCGC |
| 828 | CCGCTATGGTGGCAATCCCGATAC |
| 829 | GTTGCATGTGGCTCAGGCGGCATA |
| 830 | ATTCTGGGGAGTGACCCAGGGCTT |
| 831 | CTCTCCAAGGAGACGAGCCAATGT |
| 832 | GAAAGGACGGGATTTGGGGGCTAA |
| 833 | TATGTAGTACCTTGGCTCGCGCCA |
| 834 | TCCCTTTCGATGAGCGGCTGTACT |
| 835 | TAGATCGGGCAGAGCCCGTATCTT |
| 836 | GGAATGCTTTAGGCTGCCGAGCTG |
| 837 | ATGGTAGCAACATTCAACGCCAGG |
| 838 | CTATGAAACGTGTGGCCCAGCAAC |
| 839 | ATGTTGCTAGTGCCTTTCGGGCCT |
| 840 | CCAATGTGCGCAGACTCAGTCATT |
| 841 | GATAGTGCTCGCAAACGGGCCTTC |
| 842 | GCACCCTGTTGCCTCATTGAGCGT |
| 843 | GGCGTGAATAGAGTGACCAGGCGG |
| 844 | ACGTGCCAGCTGCGGGCACTTTAT |
| 845 | AGTGGAATAGTCGCGTCGTGCCGC |
| 846 | ACTCGCCTATTACCGCTGGATTGG |
| 847 | GAGACCGGATTGAGATGATCCCGT |
| 848 | AAAATGGCAGGCGGCAAGCAATTG |
| 849 | CTGGCAGTTTACCACCGAACCAGT |
| 850 | TTACATTGCCGATTTCGCATGTGA |
| 851 | TAAAACTGAAGGGTCGCCTCAGCA |
| 852 | GGCTTCGCATGCCTTTGCAACATT |
| 853 | AAGACCGAAGGTCTCTCTGAGGGC |
| 854 | GCCTATGGCTCCAGCTCAGCAGTA |
| 855 | CGTATCATAGCGTTCGGTGGACAA |
| 856 | CATGCGCTCGCACTCTGCCTGTCT |
| 857 | TGGGCAATTCGGAAACGTCGGTCT |
| 858 | TTGCGGAGATGCGACGGTACATTG |
| 859 | ACTTTCGCACGTCGATCTGGACTG |
| 860 | CTAACTGCCGCGGCAAACTGATTA |
| 861 | GGCCGCGGATTTTATTCCTTGGAT |
| 862 | GAATTTGGAACGGTGTTCCGATGA |
| 863 | GTCCATCCATCTACGGCATCAGGA |
| 864 | TAAACGACCTGGCACATGTGCGTA |
| 865 | CACCATCCAAGAGCCAATCCTAGG |
| 866 | ACTCATATACGATCAGTCCGCCGC |
| 867 | GTGCCAACCGACGATCAACCGAAC |
| 868 | TGGGGTTCGTACAGGTCGGTTCAT |
| 869 | AACAGTAGAGGCGAGGCCTGCGGG |
| 870 | TGCATCGAATCCGAGATGGATCTT |
| 871 | GCGTCACGTTATGTCCGCTCTGTC |
| 872 | GGGACATGCGTAGCGCAATATCAC |
| 873 | CACACGTCACACCATCCAAAGTGG |
| 874 | ATGCTCAGGTGCTAAATACGGCCA |
| 875 | AAAAATGTTTAGCGCGCTGACTGG |
| 876 | ATAGTCCGTTTCCGTTCCCAACGA |
| 877 | TCGATCTTCTGGGTTGCAGACCAG |
| 878 | GTCGGCGCAGCCGATCCTCATGTC |
| 879 | GTTGCGGGGTGTCGAAAAGGATCT |
| 880 | ATCTCTTCCTCGGGTGGATGCCAG |
| 881 | TGATGTGCGTTTCAGCTTTTCGCG |
| 882 | GTTAAGGGGTGAGAACATCCGGCC |
| 883 | AAGTCGTCTCCCTGCGTCTCGTCC |
| 884 | CCGACCTAATAAGGCGCAACAATG |
| 885 | CATCATTGGCACCGTACCAATGCC |
| 886 | TGGAGAAAGGGAAGTGCAGCAACG |
| 887 | TGGTACTCCTTGTCATGCCTGCCA |
| 888 | GGCACAGGTTCTCTTGCAGCGCGG |
| 889 | GAATCTGGGCATTGCTACGAGACC |
| 890 | CGAAATGGGAGCGTCCACTACCAC |
| 891 | ACATATGAGCTCGCGTGCTTGCAT |
| 892 | TCGAGCACGGTCACTGATAAAGCC |
| 893 | GAGGGTCCCTGCTCAGAGTTGGTT |
| 894 | AAATGCGATCGCCCCTTATGGAAT |
| 895 | CTACCCGAATGGATTGCGGATGGC |
| 896 | AGGGACTGGCAGGTCTCTGCGCGT |
| 897 | TAACGATCCATTCCACGAATGCAG |
| 898 | GGCCGCACGTACGATTACGCCTTG |
| 899 | TGGGGAATGCATCAGTTGTTGGCT |
| 900 | TATCTGGGAGTAGCAGGCAGGGCC |
| 901 | CCGAAGGTTTCACGCTCAGGTCGC |
| 902 | GAACCCAGCTGGGACATCCTTCAG |
| 903 | TGCATGCGAGCAAATAACCCGGAC |
| 904 | AATTGTCCGCCAAACGCTTTTCAG |
| 905 | GTCGGCTTCGAGCGATCGAGTGTG |
| 906 | TCGCGTGCTCTACGTAGCCCATGA |
| 907 | GGCTTCCGCGATAACGTAATTCGC |
| 908 | TGTAGCCGACTAGGGCCGAAGCCC |
| 909 | AAGCGAACGCCCTGGCTGAATATT |
| 910 | TGTCACGCGACGTGCTGCAGATTT |
| 911 | CCGTGTCCGTGTTGTCGACAGGCG |
| 912 | CCCCACACGTTGCGCCTATATGTG |
| 913 | GGCGGGCACAACTCAACACAGATG |
| 914 | CGACTGCGGGATCACCGGTGATTA |
| 915 | TCGGGACATGACCGGTACGGAGTC |
| 916 | TACCTCGAGTGGCCGTTGATCGGG |
| 917 | TAATTCATGGGGCTAGCCGAACCA |
| 918 | ACACTCTAAGCCGATTCCGTTCGA |
| 919 | GTGGGCGTGAGTGACACGCACAAA |
| 920 | ACGACTCCTCGGGCAAAGTACGTA |
| 921 | TGTGGTCATGGCGCTACTGTTTTC |
| 922 | CTTTCGCTAGCCAGAGCGGGTTCC |
| 923 | ACAGGGCGTGTTAGCGTGTGACAA |
| 924 | GGTACTTCCGGCGTATCGGGCCAC |
| 925 | GTGGGTTTTGTTCACCCTTCTGGG |
| 926 | ACGCAATTCCGCATTACTTACCCG |
| 927 | CGCCTCGACTGCGGTCAAGCACAA |
| 928 | GTGAAATGGATCCAGAGAGGGCCA |
| 929 | TATAAACGCTGCAGGGCTCCGTTA |
| 930 | GTTATTCAGGCGGCTTGTAACGGG |
| 931 | GGGTTCTAGCGTGCGCGTTCAGTT |
| 932 | TTGGGCTCGAGCGGTACACCACTA |
| 933 | CCGTCTTCAGGACAACGGTATGCG |
| 934 | GGACCCTTTGACAGATTGCGGCAC |
| 935 | TAAATTTTATCGCCAGGCGGCGCT |
| 936 | GCCGAACGCAAGATCGCTTGAACT |
| 937 | TAGGCCATTGGTGCCCTAAGACGG |
| 938 | CAAACCACAGCTTACAGGCTGCGT |
| 939 | TAAACGGAGACTGGCACGGTAGCA |
| 940 | TAGCGCGCATCACACTTGGAATCG |
| 941 | TGCTGACACAAACGAGCCGTTTCG |
| 942 | CGCTTAACGGCATTGACTGTCCAC |
| 943 | TTCCACGGCCGTGTATTACGGATA |
| 944 | TTTATGCCGTTGCCGAGGAAGACT |
| 945 | AGTGCCGAGATAGGGGACTGGGCG |
| 946 | CTAGTCTCCACGCCCTCGGGACGA |
| 947 | CCGCCATTCGGAAGATGGATGATG |
| 948 | TGACGGTGAAAGTCGATTGCGAAG |
| 949 | ATATGCGTCACCACCCGGTTCCGA |
| 950 | CCATCAGTGAAGGGGTTGCTGCCA |
| 951 | CATATGTGCTTGGCTTGCGATGAC |
| 952 | TCTGCTTTGGAAGCCTGAACTGCT |
| 953 | CGATTTGGTCAAGAAGGCGGAAAT |
| 954 | ATCAGAGGCCTTCCCGCCTCGTTA |
| 955 | ATTGTTGTCGTTGCCACATCGCAG |
| 956 | TGAAATGTGTCTGGACGCGAGTCT |
| 957 | GCGGGCGATGCTCCTTAAAGGGTA |
| 958 | CCGCAATCTCCATGCGTCGACCGT |
| 959 | TGCCGCGTAATCACCTGGAACTTG |
| 960 | TTCCAGTAGCCAGCGGTAGTGTGA |
| 961 | CTGAATTCCGCCTATTGTTCGGCA |
| 962 | GCTTGAACCTCGAGGCGATGTTCT |
| 963 | CAAGCGTGGAAGTACGACCCGCCA |
| 964 | GTGTGCACTGGATCCGAGCCCTAG |
| 965 | TCCCTGGGCTAGCATTGCGAGGTT |
| 966 | AGAACCAAAGACGCTTGTTTGCCG |
| 967 | CGTCACATGCAAACGTTCCCTCCC |
| 968 | TGACCGCATGTGTATTGAGTCGCT |
| 969 | GCGGGCCCAATGAGTATCCGTCAT |
| 970 | TAGTGACTGTGAACGCCCCTGGTT |
| 971 | GGCACCGTCTGCCGCGCGTATATC |
| 972 | TCGATGCAGTCTTTTTCCCGTCAA |
| 973 | ACCCCGTGGGGTTTCGCCATTTTT |
| 974 | CTACACGCGCAGTTGTGACTTGTG |
| 975 | CGCAGCGACCTCATCTCTGGAGCC |
| 976 | CGACCCAGCACTCCTAAAATCGGT |
| 977 | ACGCGCCGCTCATCACTACAATCT |
| 978 | CGCAACTTCCTGTGGCAAAGCCAG |
| 979 | TCGTTGGGCACATAAGGCAACTGA |
| 980 | CCGCTTGTAATTGCCATTCTCCGT |
| 981 | GTAACCAGGGAGTCCTGGGCTGTG |
| 982 | AGCGCAAGATCTGGGGGCAGTCAC |
| 983 | GCGTACATCTGCTCATCAGCATGG |
| 984 | CCTCTGTGGCAGGAAAGAAACCGT |
| 985 | CCTATGCAATGGACCTGCATCGGA |
| 986 | CTCGGTGGATGGCGAATAAGGATA |
| 987 | CCTCACTCGTGATGGCGTGACGCA |
| 988 | TACGCTCACAGAACGCCATACGCC |
| 989 | CCGGAGAAGTTACGCGGATCGGAC |
| 990 | GCGCCCTCACTGCATTTTTGGTAT |
| 991 | ACTTTCAGCACGCGAACAGCGCAA |
| 992 | CTAAACGCCCTTGATGCATGAGCA |
| 993 | GCTTGCCTTTTACGATCGTCGCTA |
| 994 | CAGACATCGTACGCACTCGGCATC |
| 995 | TAGCCGCGCGGCTCCTATGCTCTT |
| 996 | GATGCCCTTTTGGTCCCCATGCCA |
| 997 | TGAGCTGCCTTGCCACGATGCCTC |
| 998 | CCGCCGTATACGTGCCATAGTTTG |
| 999 | TAGTGCTCTCCGCGCTCATCCAAC |
| 1000 | CCCTAGATAAGTTGGGGTGGGACG |
| 1001 | TGAAGGGCCACCTGATATGGTTTC |
| 1002 | GCCGCCTCCGACTGGTTAACCCGA |
| 1003 | CGCACGGCTACTAACAGCGGATCA |
| 1004 | CCGGACCAATTCCAACGAGCATCG |
| 1005 | CATTGAGGTCCACCGTTCACATCC |
| 1006 | AGGACGCAGCATGTCCCAGCCGAG |
| 1007 | TAATCGCGGGCCATACTACCAACG |
| 1008 | CGCAAATTTCTCCGGTCGGCAAGC |
| 1009 | GTGGCTCGACTAATGCCTTGCGTG |
| 1010 | TGTGGGCGTGTTCCGGCTCACTGT |
| 1011 | GTTCTTCCTTTTCTGCGGTGGGAA |
| 1012 | ACCTCGAGTCAGATTGTGCGCCTT |
| 1013 | CAAGTGGACAGACGGTTTGTTCCG |
| 1014 | TCCAGTTGAGTCGCGCCGACGAGG |
| 1015 | CGCAACAGGTCAGCCCTTATTTGC |
| 1016 | GCCGTGACTCCTGCAATGTCGGTA |
| 1017 | ATCAGCGCAAGCTGGTCTGAAACA |
| 1018 | CCCTGGCCAGAACGAGAGGCCATG |
| 1019 | ACGATCAAGGACTCGTCAGGGTTG |
| 1020 | TTCATGGCACCAAGACCACCGTTA |
| 1021 | ACAGCAAGGAGATGGATTGCGACG |
| 1022 | CGTAAATATCTGCGGCGGTGTGAA |
| 1023 | GGAAACACGTGTTCGTCTGTTGGC |
| 1024 | CGATGTTAGGATTCGGATAGGCCA |
| 1025 | ATCGGACAAGGACAAGTGGATGGT |
| 1026 | GCCCGGAGGACAAAGTTCGAGTTA |
| 1027 | AAATCCGACAAATGGGCACATGGA |
| 1028 | CAGTTAGGGGATGCGGATGAGTGA |
| 1029 | CGGCAGGTGGAGATTCCGACATTG |
| 1030 | TAGGGCAGCCAGGTTCACTCATCT |
| 1031 | GCACCGTATTAGCAGTAGGCACGC |
| 1032 | ACGCATTACAGGTGTGCGAAGGGA |
| 1033 | CGTGACTGCACGTGTTCCACAGGG |
| 1034 | GCTGAACTACCGCCTAAAATCGCG |
| 1035 | AGCACGCCAGGGAGGATCGAGTTA |
| 1036 | ATGAGGGCAAGGAATGGGTCATGC |
| 1037 | GGGTCTCTCGTAATCAAAGGCCGA |
| 1038 | TATCTTGCGCAACGCCTCCATTTA |
| 1039 | GGTTACACCTACGGAATCCAGCGG |
| 1040 | ACACCGAGTTGGTCCGGTCAATAG |
| 1041 | TCCCAGATTAAACGCTAGCCACCG |
| 1042 | TTGGTGAAACTGGCCCGTCGGAAG |
| 1043 | CCAGGGGAGTTGACAATGAGGCTG |
| 1044 | TCTGCGTTATTGGACCGTTTGTCG |
| 1045 | TATGGGATGCTAAACCGGCGTACA |
| 1046 | CACAGACGTCTGTCGGGCTTGTGT |
| 1047 | AGAATGCCGTTCGCCTACTCCCGT |
| 1048 | CGACGGATAATGCAGGCCTCATGA |
| 1049 | ACCCTCTAAAGCAATAGGTCGGCG |
| 1050 | CACTCACGGCAGAAGCCTGCTTGT |
| 1051 | ATCAGCCCACATATTCTCGGCCGT |
| 1052 | CAAATCTGGGGTCGTCCTAAACGC |
| 1053 | TGTCGCCCATGGCAGGTTAAATAC |
| 1054 | GGGGGCCCATCAATTCATTATCGA |
| 1055 | GTCGAGCAGCTTTAGTATCGCGGG |
| 1056 | CCGCTAAGCACCGAAGGCTCACAA |
| 1057 | TAGAATTAGCGAACGGTGATCCCG |
| 1058 | CACATGACATTTGGCAAAGGTCCA |
| 1059 | TCAACGCACTGGCGATGACTAGAT |
| 1060 | CGGGAAATGTCTTTAGCCGTCGAA |
| 1061 | ATCAGAGCAAATCTGCAGCGGGGA |
| 1062 | GGCCTGTTTCTGTCCAACTGGGCT |
| 1063 | ATTTCACCTCGCTGATCGCTTCCG |
| 1064 | AGTGACGCCGAGTCGCGAGGGTTA |
| 1065 | AGTTGTCTCATCCTGTCCGGGACC |
| 1066 | CTTCTTTGTGCACACTTGCCAGGG |
| 1067 | CACCTCATCGGAGCATAGCAACCC |
| 1068 | ATGCGATCCATGACAAGGGTTGCT |
| 1069 | CCCGTGGAGATGATGTGCGGCTTA |
| 1070 | CCCAATAGACGCCACAGCCAGTGA |
| 1071 | AACGACCACGACCCTCGCCGAGTA |
| 1072 | GGTGCTTTGTCTGAGGCGAGTGAA |
| 1073 | CTGTCGGCGCTGCTCTCCGAATTT |
| 1074 | CTCGCCGGAGTGTTGTAAGCATTG |
| 1075 | AGCAATCATGAGAGGTGGCCGGTG |
| 1076 | ATTTGCCACCGGCGACAAAAAGAT |
| 1077 | CCGCCCGTGTTGGCATGTCTTTTG |
| 1078 | ATCGGAAGTGCTGACTGACACACG |
| 1079 | CCTCAGACCCTATCTGGGTTGACG |
| 1080 | CTGTGTGGTCTGGTCCGGCTGTTC |
| 1081 | GTCCCCATTATCGGTGAGTGCAAC |
| 1082 | ACAGGCACGTAAGTGCTCAATCGG |
| 1083 | AGCAAGATAGCGGGAGTGCCCCTA |
| 1084 | GGTTTACGCCATGACATCCCGTCA |
| 1085 | GTGCAGGCCTTTGTGTGTGAATCG |
| 1086 | CTTCGAGGGTAGGGCTTCGAAACG |
| 1087 | AGTCGACACTTGGGTTTACCACGG |
| 1088 | ACATAAATCTCGCCCGCTGCACTC |
| 1089 | GTTTGGTTTTCCACGGAGGTTTGA |
| 1090 | GCAGGAACCAGATTAGTGTCCCGG |
| 1091 | TTTGCTAGAGCGCGGAGCTAAAGC |
| 1092 | CTATGTGGCATCGCTGACATGCTC |
| 1093 | CCTAAGTCGGTTTGCAGCTGCTCT |
| 1094 | GCGTTCGTCCACAGGAACGGAAGG |
| 1095 | TAACCCGCGCCCGAGAAATTGTCT |
| 1096 | TATGGTGCTCAGAGCTGTTGCCAA |
| 1097 | TCATCGACCCACTAACGTCAGGGC |
| 1098 | TGCTCAAGCTACGCGTCACTTCCC |
| 1099 | AGCGGGAAGGTCTGAGGAGGGAAA |
| 1100 | CCGATGTAGCACCACCGCAGTGGC |
| 1101 | AAGTTCTGGGAATCACACGGCGCG |
| 1102 | CACCAGCCTTACGTGCGGCGTTAA |
| 1103 | CGTTTCGCCTCCTCTTCCGAATGC |
| 1104 | GAGGAGGCCAATAGAGCAGCGCGC |
| 1105 | AGTAATCTTGCGGCACACAAGCGG |
| 1106 | TGAGGACAAACCGCGCGTAGGATA |
| 1107 | TCGTAGAGACGCAGTGCCCATCTC |
| 1108 | CGAAGCTACACCCCGAGTGCGGTG |
| 1109 | ATGATGTGATCTTCCCATGGCTGG |
| 1110 | TGTACACGTATCGCGTTCGCCTAG |
| 1111 | GGTGTGCTTTTACGCATGTACGCA |
| 1112 | AGGCGGGATACGTGGATGCTAGCC |
| 1113 | AAATTAGGCACAGCCCTCCCACAG |
| 1114 | ATAAGTTTGGTGAGCCATTCGCGA |
| 1115 | CCTATTTCGGCGGACCTCGATGCC |
| 1116 | TTACCGGAATATGCACTTGGCCGC |
| 1117 | CCTCTCGGACGGTCCCTTTGATCG |
| 1118 | CAAGCGAATGCTGTATTACGGCCT |
| 1119 | GCATTTCCCATGCCAGAACGTTGA |
| 1120 | GTTTTGGCTAACCGTCCTGCCTTG |
| 1121 | AGGTTTTGTCCGGGCGAATGATGT |
| 1122 | ATGTCCACGAGTGCGTCCGATATC |
| 1123 | AGACGCGTACGAGGGTTCTGCGCC |
| 1124 | AATACCGTTCCCATCTGTGCGAGG |
| 1125 | ACACAAGGTGCCTCATCGAATGGT |
| 1126 | GCCGGCAAAATCCTACAAAATCCA |
| 1127 | CTTATCCCATGTGCCGGTCTGACT |
| 1128 | GCGGCCATAATGCATAGCACGGAA |
| 1129 | TACGGTGCATCGCAGTATGGGTAA |
| 1130 | CACCAGATGTCGAGGATCATCGCC |
| 1131 | GCTCCTACGCCCAAAGAGGTATGG |
| 1132 | AGAATATGGGCAGCAGCAGCACTC |
| 1133 | CTGCAGTCGCACGCAGTAGACCCG |
| 1134 | ATGTCCCTGACCGGAATCTTTCCA |
| 1135 | TTCGCCACGAGGCATTAGTCCGAC |
| 1136 | ACGTCGTTCCCGAGAATACGGTCT |
| 1137 | ATCCGCTGGCGCTTTGACGAAGAA |
| 1138 | TGPACCAAATTCTTACCGCGTGGA |
| 1139 | CACGCGTAGGCTGGTGTGTCATTC |
| 1140 | TCGATCCCGCGATCTGGCCTATTG |
| 1141 | GGAACACTCAACCACCGTGGATCT |
| 1142 | TCACACACCAACTGGCCACAGATG |
| 1143 | TGTGCTTAGGACACCAGGCAACCC |
| 1144 | GACATTTAACCCGACCGATTGTGC |
| 1145 | GGCACCGAGCCAGTAGGCCTCTGA |
| 1146 | CTCAAGCGTGCATGTTGGTAACCA |
| 1147 | AGGAAGGCCACCATCCAATATTCG |
| 1148 | TTGGAGCCCTGACTGAACCAAATC |
| 1149 | TACGAACGCCAAGGTTATGCCAAT |
| 1150 | CGCACCAGAGTTATGCAGGCTCAA |
| 1151 | CCAGCTTGGACGAGGAAGGATGTG |
| 1152 | GTCACGCCTTTCAAATGACCCACA |
| 1153 | TGCTAGACCCAGCCCGAGTCTCGG |
| 1154 | TATTGTGGCACTTGGGTCCAGTGC |
| 1155 | CACGTGTGAGACCGGAAGTGCATC |
| 1156 | AACCTCCAGCAAAACGTCGAGGTT |
| 1157 | GGCAGCCTGATGCTACAGCACCGT |
| 1158 | CGGTCCGTCCATCCTTCAGAGTTA |
| 1159 | CTATTCGCGGACCCTACGCAGTTT |
| 1160 | ACCTGTGCAGTCAGCACGAGTGCG |
| 1161 | GAGAACCACAGGTGGTCCACCCTA |
| 1162 | CCTCGCTAGAGAAATCCACGGGAT |
| 1163 | TAACATCGGTGCAAACCGTGGCGC |
| 1164 | ACCCAGAAGACATGGCATTCGCCT |
| 1165 | AAAAGCGCTGCTCTAACACCGCCG |
| 1166 | CAAGTCTGTCCATTTCCCAACGGT |
| 1167 | CCGACACATGGTGGGCTTTTTAAG |
| 1168 | ACAGACCAGCTTTTTGCGCAGATT |
| 1169 | CGGCGATCCATTTCACTTCAAAGT |
| 1170 | GACGTTATCATGACACAGGTCGCG |
| 1171 | GGCAGAGTTGGATCGGATCCTCAA |
| 1172 | TTGCTGGCAAACAGCTCCTGAAGA |
| 1173 | CCTCAATGCCACCGAATTCGGTAT |
| 1174 | GGAGTTAGCGTGATTAGTCGCCCA |
| 1175 | GAACTCGACGTGTCACGGAAGGGT |
| 1176 | CACAAGCGACATTTCTGGTGCACG |
| 1177 | CCAGAATGCGTGAATTCGCGTCCT |
| 1178 | CAAGGGAGCCCTGCGAATTAGAGT |
| 1179 | ATTCTTGCTTCGGACGACTAGCCG |
| 1180 | TGCCACTTTGATTTCCAGATTGCC |
| 1181 | GATGGTCGGCAGATAAGTGGTGGG |
| 1182 | GTTCACACGGGTTGACCAACATGT |
| 1183 | GATTCAATTGCCCCATTCCTGCAT |
| 1184 | TACCGGAAACTGAGCCTCGTGCTA |
| 1185 | GGATCTTTACTCAGGGGCAGAGCC |
| 1186 | CGCGAGTGCTTTGTTCTGTGTGGA |
| 1187 | GTCGTCGCGATGGCGTACATCCTT |
| 1188 | ACGGGAATCTCCCGAAGTGCGAGC |
| 1189 | GGTCGAAATGAGCCAGCAGCAGAT |
| 1190 | CCATTGGAATACTGCGTGCGGCTT |
| 1191 | GGAAGACTTCGCGAGGGCACAATG |
| 1192 | AGGGTGACTTCGAAGGTCCGAACT |
| 1193 | TCGTCCCTCTGGTGGTCGAATCAC |
| 1194 | TGTGCAAATTATGCTGGGCGTGAG |
| 1195 | GTCGCCAACTGTCATGTGTGCCCA |
| 1196 | CCTCGAACCCTCAAGACGAAACGA |
| 1197 | CTTCATCACGTGACCTTTGTTGCC |
| 1198 | CCTTCATTCCCAGCAGGATGGCTT |
| 1199 | CGGGGACCTCAATGGAGCGTCTTA |
| 1200 | CGCCTCTAGCGCTTGTTACGTCGA |
| 1201 | CTGCCAGACTCAAAACAGGGACGG |
| 1202 | CTCCTTACACCGTGTGAGGGAACC |
| 1203 | TTTCATGCCATATCGCCTCGCGCA |
| 1204 | TCTGGCTTTTCCTCGATCAATCGT |
| 1205 | GTCTGACTGTCTGCCCTGTATGCG |
| 1206 | GGTTAATGGAACGGCGTTAACGCG |
| 1207 | CTTCGCACTGCGGAATCTCAAGCT |
| 1208 | TGCCAGAGGCGTAGGAGTCCTGGA |
| 1209 | GACGGGCGAGCCAGTATTAACTCA |
| 1210 | GACCTCCAAAGTCAGTCTTGGCGG |
| 1211 | CGTTAGAGCATGACCGAACACGTC |
| 1212 | GTGGGCTCAAAAATTGGGTACGCC |
| 1213 | GGGGCAGAGATCACGCGTTCCTCT |
| 1214 | TTTCGCCCTACGAAGCGAAGTTTC |
| 1215 | TACGGGGTGATGTTAAGCTACGCG |
| 1216 | CCTGTGAGTCTGAGATCGCCGTGT |
| 1217 | ACTGAAGCTGGAACAGGCCATTCG |
| 1218 | AGCACTGGTTCACATGGGAGTCCA |
| 1219 | TAAGGAAGATCACACTCCCTGCGC |
| 1220 | CACCACACGCTAAAATTGAAGCCG |
| 1221 | GCTGTCGCCAGGATCATGTATCGT |
| 1222 | TTCGTTCGTGCACTGGATTCTTGA |
| 1223 | TCAGCTCTCCTTGTGCTTGCAGTG |
| 1224 | ACGACGAGGTGAACTTCGTGGGAA |
| 1225 | AGCATTGCCGCGGGCCTTGGTTTA |
| 1226 | CAGAGGGCAGATGTGACTCCTCAA |
| 1227 | CGATATTTCAGCCTCTCAAACGCG |
| 1228 | TGCCAGAAATGTTGCCGATTCGAA |
| 1229 | TAGGCCACCCGGTGTTCACAATTC |
| 1230 | GAGAGTCAGACCGAGGGACACGAG |
| 1231 | GAGGCGATCCTGGAACCACGCAAC |
| 1232 | CCAGAGAGGCGGGCTACTGACTCA |
| 1233 | CACACAGTCCCATCGTACGGCAGT |
| 1234 | TTACGTTGCGGAAGCGTGCCTCTA |
| 1235 | ATGTACACGCTGCAATCGTGTCCC |
| 1236 | ACTCGTCGTCGGAAGCGCCCAGGT |
| 1237 | ATGCGAGAGCAGAATTGAGCCGGT |
| 1238 | AAGTTGGTTCGTATTCACGCGTGC |
| 1239 | TGGGCTTATCGCCGAAGATTGCTA |
| 1240 | CAACGGCGAAGACCCAGAATTTTA |
| 1241 | AGCGTACGGCGAAAGTCTAGGGAC |
| 1242 | ATGCATCCAGCGTCCCCTTGATTA |
| 1243 | ACCGTCATCAGTCGCAGGCTTCTG |
| 1244 | TCTTGACGGCTGGGCATGATTGGA |
| 1245 | TTAACATTCGGACCCAGGACCTGG |
| 1246 | TGGTGTCGAACTCCCTTGCGTGTT |
| 1247 | TACTCCAGTCGCCTGCGCGCAAAC |
| 1248 | CGCAATGCCGTAAGCATGCCAAGC |
| 1249 | AGTCCGCGCGAAATACGAACAGTA |
| 1250 | ATGTTGCACGCGCACTGTATCACA |
| 1251 | GGGATCAGCATCATTGGAAAGGAG |
| 1252 | ATCGCCTAACTACCCGCGGCGTGC |
| 1253 | TGGCCAGGGAACACAAGCTCGGTA |
| 1254 | AAACATGGGTCGCGTCTGAGATCA |
| 1255 | GCGAGAGCTGCGATTCCCTTTTAG |
| 1256 | CCGGCCAAACAAGAGACGAGCGGA |
| 1257 | AATGGGGCACAGTCTCGCTTGACA |
| 1258 | TGTCTCGGGCCTTCAGGACACACT |
| 1259 | TCCACCTTCATTAAGTGGTTCGGC |
| 1260 | GCTTCGGAATCATCCACCTGTCAT |
| 1261 | GAGCCGATGGGCTATCGTCGTCGG |
| 1262 | CACGAATTACGCACGCACAGAGGA |
| 1263 | GCTGTGACGCTCCCCTCAACTAGG |
| 1264 | CGCTCTGAAAACGCGGGCTACGTT |
| 1265 | GAGTGCTGGACACCGTAGCCAGGA |
| 1266 | CCAACCCCAGTGTAGGCGCAAATG |
| 1267 | GAAGTAGGGGATGTTGGCCGGCGG |
| 1268 | CAACGTGGGCACCTGTTTTAGCAG |
| 1269 | CTAGCTGCGATCCGAACCTCTACG |
| 1270 | CATTGAACCATCAGCCAAGCTGCG |
| 1271 | AGACTGGCAATTTTTCGAGGCCAA |
| 1272 | CTGGCCGTCCATGAGTTGGTCCAG |
| 1273 | CATGCTGAAACACGGGATTGCCAT |
| 1274 | CGATATGTAAGACAGCCGTCGCAA |
| 1275 | AGCGTAACCTACTGGGAAGGCACC |
| 1276 | GTGCTCGTGGCACGTACAGGCCTT |
| 1277 | GTTCGAACCCCGCGATGTTAAATG |
| 1278 | GTTGTTAGGAGGCTCGAGGCTGCT |
| 1279 | ACTGGTGCTACGCGGGATATTTGA |
| 1280 | CTGGGAGCTATCCTCAGCCGAATC |
| 1281 | GAACTCGCCGCTGCCGAAGGGTAG |
| 1282 | TTCGATCGAGGAGCAAGGAGAGTC |
| 1283 | GGGGAAAATTGAGGCCTTAGCCAT |
| 1284 | CTAAGGTCAAAGCGCTGTCGCCAG |
| 1285 | GTGAGGCTTACCCCGTGCTCTTGG |
| 1286 | CCGTAGCGGTGCTCGACCAGGTTC |
| 1287 | TGGGGACGAATCCGAATGTAGTGA |
| 1288 | GTCATGTAATTGCATCCCACGGGT |
| 1289 | CTTTGCGCGGTGGTCAATAAAAAG |
| 1290 | CACTCGAGATTCAATGGGCATGGT |
| 1291 | CTCGGGGATGCCCTCTTGGCATTA |
| 1292 | CGAAACGTGGTGCAGAAACCTGAA |
| 1293 | GGAGTTCACGAGTCGAGCAGTCGC |
| 1294 | AGCCGTTTTCAAAGATCTCGACGA |
| 1295 | TGGCTGGACATTGTCTGCAATGCA |
| 1296 | ATCGGCTGCCTCAGTCCCTAATTT |
| 1297 | CCAGCATGGAGTTAAGTGAGCGCG |
| 1298 | TTCATATTTACGAATGCCGGGTGC |
| 1299 | CGAAATCGCACAGGAATTCGCGTC |
| 1300 | GGCAATTTCGGGACACTCGTTTCA |
| 1301 | TTTGTGATTGGGGGTATAACCCGA |
| 1302 | CCCAGCTAATCCAGCTTGGGCTGT |
| 1303 | AAAATCGTTTGGCTGTAACGTCGC |
| 1304 | AGGAGATTCATCGACTTCCGGGAA |
| 1305 | GCACGGGGTCTCAATGCTTAGGGT |
| 1306 | GCGCAACAAGTAGCCTACCGAGGC |
| 1307 | TAGCAGGCTGATGCCGTCTACACA |
| 1308 | GCAAGCGGCGATCGTACAACTTGT |
| 1309 | GCACCTCTGGTAAGCCTGAAAGGG |
| 1310 | CGAGGGCGGTGAGTGCATACCGTG |
| 1311 | GGATTAACCGGAACTGCCCTTCTG |
| 1312 | GATATTGGGTCCGGCGCGCATTAC |
| 1313 | GGCCTTTAATCTCCGGTCGCAATG |
| 1314 | AACCTTAGTGCGGCTAGGTGGGGT |
| 1315 | CACGCTGACGCCAGTGTGGTGAGG |
| 1316 | GGTTCCCTTGACCCACCGAATTGA |
| 1317 | TTCTGACAACATCGACCCTGGCTC |
| 1318 | GCGAGCGAAGATAATCCCCAAACT |
| 1319 | GTACTCTGTGCAACGGTCCCGAGT |
| 1320 | ACACGCCAGGAACAGTGTCTGTGA |
| 1321 | AAGGGAATTTAGCGCGCGTGACTT |
| 1322 | TGACGTACGCGTTTTAAGTGGGGA |
| 1323 | CTTAGAGGGACGAGGCCATGAATG |
| 1324 | GGACGACTCCGCAAAAAAGGTCGT |
| 1325 | TCAATCCCAACATCCAAAGCCTCA |
| 1326 | GCACTGGTCTACCAAGCTTGTCCC |
| 1327 | ACTTGTCGGAAACGAGACCGAGCA |
| 1328 | TCAGGAAAGGCCTAAAGGCGAAAG |
| 1329 | GGAATGTAGTCAAGGAGGACGGGG |
| 1330 | GCACGTGGTAAATGAATTGGCGAG |
| 1331 | GATCATCAGGGGTTATGCGTCGCG |
| 1332 | CTCACTCATTCTGATTGCCCGCGG |
| 1333 | GGGGTGATCTCTCGAACGTCACCC |
| 1334 | AAGGTTGCTGCTAGCGTACCTCGA |
| 1335 | TATAGATCGCCCAACAGGCAGGAG |
| 1336 | GTTTGGACCTGTTGGGAGTGGGCA |
| 1337 | ATTGGGGAAAACCCGGTCTCAAGG |
| 1338 | TCGACGATAAAGTGCTCACGGGAC |
| 1339 | CGATAGAATTCAATGCAGGGCGGA |
| 1340 | CGGTTCGCTACGGCGGCTGGTTTC |
| 1341 | CCAGGTTTCGGTTAGTCGCGCTAG |
| 1342 | ACGACCTTACACTCGGATCCGACG |
| 1343 | TCGCGTTAAATGGACCAAGGGGCC |
| 1344 | CCAGAAAGAAAATGGCGCCCGGAT |
| 1345 | GATACATCGCCGCCTGCTAGGCAC |
| 1346 | GAGATCACACTCGGAAACCGGATG |
| 1347 | ACTTCGCGGAAAAAGGCTGGCATT |
| 1348 | CCGAGCTGCACGAGCACACAAAGT |
| 1349 | TTCCACAAGGCGGCATAGTGAGGC |
| 1350 | AGCAAACTGGAATCCGGAAAAACC |
| 1351 | CGCTATGTCGCAGCATGCATTTAC |
| 1352 | AGTCACGCCCAACGTCGGTTCTTT |
| 1353 | AGTGGGCGCACTTGGCCTTAAATA |
| 1354 | ACTTGCAACTTCGGCCGTTTGACT |
| 1355 | CAAACATCAGGTTCATGCCGTACG |
| 1356 | AGCGTGACCACCCTACAATGGCAA |
| 1357 | GCAGGCATCCGGCAGAGATGTCTC |
| 1358 | GAGCGGCTAAGAGGCCAGACCAAA |
| 1359 | CACAGAACAGGGTGTTTCCCGCTA |
| 1360 | ACTTTGCAGAAGGCCCAACACAAG |
| 1361 | CCTTCCTGGTACTTTGTGGGCGAC |
| 1362 | CTACATGCTCACCCCACCAGAGTG |
| 1363 | ATTTTCAGAATAGCCCCGCCTCGA |
| 1364 | CAATTGCTACGTTGACGCCCTCTG |
| 1365 | CTGTCGCCTAATCCTCGGTGGCCG |
| 1366 | TTTGTGTTGGCTCCGTACATTGGA |
| 1367 | ACGTGACGGGAAGGTGGTTGAATC |
| 1368 | AGTTCTTGCGTTGCACGAAACAGA |
| 1369 | GCTCGCCGCGCGTCTTTATGTCTG |
| 1370 | ATGAACATCGCGAGGCAAGCCTTT |
| 1371 | CAACCGCGCCCACCAACATTAAGG |
| 1372 | TGATCGAGGACGGCTTGGTAGCCT |
| 1373 | GGAGGCATGCCTTCCGAGAGCAAC |
| 1374 | CACCGATCCTCAACGCAATTGCTA |
| 1375 | GGCCATGAATTGGGAAATCCATGT |
| 1376 | CTGTTCCAGGCGTAACCAGCGGGC |
| 1377 | TATGTCTGGCTCGCCATCAGAAGA |
| 1378 | GGAGTGACCAGCACAAGCATCGAG |
| 1379 | TCGGACTGGAAGTAACTCGCATGA |
| 1380 | GTAGGGTCAAGCACGATTGAAGCC |
| 1381 | CACCGGCGGTTCGACTAACGTGAC |
| 1382 | GAATGACGCGCAGTGCATTTGAAC |
| 1383 | GTGCTCGTCTAACCGCGGATAGAG |
| 1384 | GCGGACCTGGGTTAATTGACGCGC |
| 1385 | TTTTTGATGTTGCGCACCGGGCTA |
| 1386 | TTGCGTCAGCGCATCTGCTCGATT |
| 1387 | ATGAGCACGCCAGTTCGTTCCTTT |
| 1388 | TCAACGGTAAAGAATCGCCCCGCA |
| 1389 | CGCGATTGACTGAACCACACCTCT |
| 1390 | GCGTGXAAGATGACGGCCGGTATA |
| 1391 | CATGATTCCACCTCGATCGGCTAG |
| 1392 | CTACGACAAAGCAACCGTGCAAAA |
| 1393 | ATGCCGTGTTCATCTTGATGGTCC |
| 1394 | TTCGTGGAGGGACTTTGGAGATCC |
| 1395 | GAAGCGCCGTAACGTACACCGTCG |
| 1396 | AGCGTGCGCTTGGCTATAAGGCTA |
| 1397 | ACAGTCAGGAGTAACGCCGCTCAA |
| 1399 | ACTGTGTCGCAATCAACCCGCAAA |
| 1400 | TGCAGCCAATGCGGAACTTAGAGG |
| 1401 | CCCGCTATCCCGGTCTTGCAGTTC |
| 1402 | GAGGGCGCAACATATGCAGTGCTG |
| 1403 | CGTACGGACATCGATGACGCAACG |
| 1404 | AGTCTCCCGAGAAACGCATAAGGC |
| 1405 | AGGAAGTGGATGAACGCGGCTGCA |
| 1406 | GGGTTGCTCACCCTCGTCATCAGG |
| 1407 | TAGGAATGCGAGTTCCGGCGGTAA |
| 1408 | CTCCTCACTTCCAAGCTGCGGATA |
| 1409 | TCAATAGCACCTAGCATGCTCCCG |
| 1410 | TGATTCCTGCGCTTTCACAGGTCG |
| 1411 | GTATGTGCGGGATGGAAATCACGC |
| 1412 | TACGGCAACTGTCGATACGAGGGC |
| 1413 | GGTTCCCTATCCAGCACTCCTCGC |
| 1414 | ATAAGCGCGCCACAGGTATGTACC |
| 1415 | GAAAGTCGCCAACAGACTCGAGCA |
| 1416 | CGCTAATGCCTCATAGGCGTGTGC |
| 1417 | ATCCCCGCCGCACGAAGTACCAAG |
| 1418 | GACGCTGCTGATGGCTTTATCGAT |
| 1419 | CTCTCCCCGTCGCTTCAGAGATTA |
| 1420 | TCATGTGGGCCGTCGTATCAGTTT |
| 1421 | GGCCTGAAGGTGAATGGTTACGTG |
| 1422 | AGCCTCCAAAGCCGGTAGAGTTCC |
| 1423 | TTGTCGTAGGCGCTCACCTTAGGA |
| 1424 | GCCTGAGTCCGGGTCGGGAAAGAA |
| 1425 | GGCACTATACCGGTTCTGGACGCG |
| 1426 | CCGTGTATACGGAAAGGTACGCCA |
| 1427 | CCCAAGGCAAGTGTGCATCAGTCC |
| 1428 | GGAGTGCATCATGGCCAAATCTGG |
| 1429 | CCATGTTACGTCTGCGCACCACAG |
| 1430 | GGCGTTGAGCTTAAAAGCAGCGAC |
| 1431 | TTGGCACTCTGCAAGATACGTGGG |
| 1432 | GATCTGCACTGCAAGGTCTTGGGG |
| 1433 | CGATCAACTTGCGGCCATTCCTGC |
| 1434 | CGGCTGGGGTCACAGAAACGAGTA |
| 1435 | GCGGCTAGTTGTACCTAGCGGCTG |
| 1436 | TCGTCACTGTTAGAGAGGCCTCCG |
| 1437 | AGTGTCGTGAGCCCTAGCGGCGCT |
| 1438 | AGGACGCAGGGATTCAAGTGCAAC |
| 1439 | ACCGATGCGCGGTCGGTCTCATAC |
| 1440 | GGCAGAGGGTTAGGGGGTTTTTTT |
| 1441 | GGCAAAGGGTGTTTATGGGAGACC |
| 1442 | ACAAGGCTTCGGCTGGCAGAATAC |
| 1443 | CATATCCGTTCCTATCGCCAGACG |
| 1444 | AAGCCTTTGTGGCCAAGGCCGCGT |
| 1445 | CCGAACCATGGCTTTATCCAGTGT |
| 1446 | GTTCAGCAGTAGCTCCCTCCTCGA |
| 1447 | GCGCAGTGACACCATGATGC1TVC |
| 1448 | ACGATCCATTTTGCCAGCATGCAA |
| 1449 | TCCCTTCATTTCGGGTTTTTAGCC |
| 1450 | TCTTCTTGCCCACATTOCCTTTTG |
| 1451 | TGCCTTTTGATTGGTGGTCACGGT |
| 1452 | GACCCTCACGGTCATCAGAGGGAG |
| 1453 | CCGTTCAACACAGTGATACACGCG |
| 1454 | CACCAGGGGATAGGTGCGGTACGC |
| 1455 | GGTCGGAACTGATCTGTGCGATCC |
| 1456 | TGCTCCTTCCTAGGGTCATCCGTG |
| 1457 | GTGGACTTTGACGCCGGCTACCGC |
| 1458 | CTGATCTGTCGGCGGTTACTTGCC |
| 1459 | AGAGGAGCGGAAAAAACCGGACGA |
| 1460 | GCGACGAAGAGATCCAGCAAGCTC |
| 1461 | GGGACTTCCAGCTGAGGGACGAAA |
| 1462 | GGCGCACTCCAATACCCACTGTTT |
| 1463 | GCGCTTGGAGACTGTCAGGACGTG |
| 1464 | CAAACCGCTGGTTTCTCCACCTGT |
| 1465 | GCGATTGCTTGGGATCGGTGACTA |
| 1466 | CTCAGCGACATTTTTCTGGTGGCG |
| 1467 | CAGCGGCGTCGTTTACTCAGGACT |
| 1468 | GACAGCCGTGAACGCTCAGCCGTT |
| 1469 | GGGCCGTAGAGGCATCGGGTAAAG |
| 1470 | CGCCGCTCACCTGCTTAAAGCATT |
| 1471 | TGCCAAATCGCAACTCTTGAGACA |
| 1472 | CCCCGATCGGGTGTAATTCTCCCT |
| 1473 | CAAGGTCCAGGTGACGCAACCACT |
| 1474 | CGAGCCTTCAGTGGTATGCATGCG |
| 1475 | CAGCAGCGTGCCCATCTCGACTTA |
| 1476 | CGGACCAAGATGGCAGTAATCCAG |
| 1477 | CTACCACGCTCTGCGCGGGCTGTA |
| 1478 | ACGTGGTTAGGCATGAGCTGCGTC |
| 1479 | CGACATATCCGACATGACCGGATG |
| 1480 | GCGCCCAGGCTGTGTTAGAAAATA |
| 1481 | AGCTGGGACTCCGGACCTTGAGTG |
| 1482 | CGGTCGTAACCGCTGCTACAACTT |
| 1483 | TCGTTCCTCTGGAACAATTCAGCA |
| 1484 | CGGCATCTCCGGACAAAGGTTAAC |
| 1485 | TATCTTGTCGAGCGCCACTCGGAG |
| 1486 | TGCAAGGGAGAAAGCCCCATGAGC |
| 1487 | ACTGCATAGCCCAGATCCGCTTGC |
| 1488 | TGTGATTCAGTCGAAGCAAGGCCG |
| 1489 | CATCCATCTACAATTCGGGCCAGT |
| 1490 | ATGAGCCGTTCAGAAAGCCAAAGA |
| 1491 | ACACTGGAATTGCTAGACCCCGCG |
| 1492 | CTGAGCTGCGTGGGACAACTCCGC |
| 1493 | CAGCTACTAGGGCGCGATGTACCC |
| 1494 | ATAATGATGGGACGAGAAGGCCCC |
| 1495 | CGACCGAGTGTTACGACATGGTGC |
| 1496 | TGCAGTACCCGCCGCTCCACTAGT |
| 1497 | ATGCTAGCGCGCCTGTCAACGTAC |
| 1498 | AGACTCACTGCCGGCTGATCAAAT |
| 1499 | GCCTGGTGCGAAGATAGGGATTCC |
| 1500 | GGAAAGTTGGCGGATCCGAGCACT |
| 1501 | GGCAGTGAGCAATGTGTGACGAGG |
| 1502 | TGAGGTCCTCCCGGCGGACTACGA |
| 1503 | CTCGCCTTAGATCGTGGTTCCGCA |
| 1504 | GTCGAGGAATATCATCGCAGCCAG |
| 1505 | GCGAATGCAACGAGACAAGAAGGA |
| 1506 | TTCGCCACCAAGTCGGCATTTGTT |
| 1507 | CGGTGGCTGACACTTGCCGGATTC |
| 1508 | CAAGGAGCAATCAGATGGTCGGAG |
| 1509 | GTGACCCGGTCCGTTCTAGCTGTG |
| 1510 | CTCTCGCCCACATAACTGCACAAA |
| 1511 | AAACCTGCCTAAGCAAGCACTGGA |
| 1512 | TTCCATATTGTACCCCGCGCATGC |
| 1513 | TGCTTGCGATATCACGATACTGCG |
| 1514 | TTAGTGTTCGAGCCTTGAGCCGGC |
| 1515 | CTTGTTGCGCGAGTCCGTCTGGGA |
| 1516 | GTCAGCTGCCTGCTGGTGCTCTTC |
| 1517 | CATCCCTCGAGGTGTAGGCAACAC |
| 1518 | CAGATGCACTCCGACGGGATTCAG |
| 1519 | CTGAGCCTCGCGAAGCTGTGGCAT |
| 1520 | GCTATGCCACGCCGCAGATAGAGC |
| 1521 | AACACCAACCATACCGTCCGTTCA |
| 1522 | GCCCAGAGCTAAAGCATGTCTGGG |
| 1523 | AATGCTGCAATGCTAGCGTCGCTA |
| 1524 | TCCGGACCCACTATCCAATCCCCA |
| 1525 | TAAGACCATGTGGCACCAAGGTGC |
| 1526 | ACAGCCACACACACGCGCCCACTA |
| 1527 | TAGAACCGAGCACGGCGCCTTGTA |
| 1528 | TTCGAGTAAGCTGGCAGGACCACT |
| 1529 | CTTTCGCAGGTTCGCAGACAATCC |
| 1530 | TACGTCCTGTGCTGTTGACACCGG |
| 1531 | GTTCGGGTCAATGTTTCGGGGAGA |
| 1532 | CCCTGTTGTGAAGGGGTTTTGTGA |
| 1533 | GGCAGATTGGTGAACCCCAGATAA |
| 1534 | CCCTCGGTGTGTTCAAGCCAAATC |
| 1535 | CCCGCGAACATTTGAACAGCTTAA |
| 1536 | CCGTGTCAGTTGCTCCCTGGCACG |
| 1537 | TCCGTCTCAGCCGCCTCCCTATCC |
| 1538 | ATAGCTGGGTCACCACAGGCGGTC |
| 1539 | ATAGGCAAGCGGTGTAGCACAGCG |
| 1540 | TTAGAAGCCGGTCTGGATTTGCGT |
| 1541 | TGCCGACCTTTACCAGGATCCTCG |
| 1542 | GCCCACACTATAACCAAGCTGGCA |
| 1543 | TTGCGCCACTAGTACGGATCTCAA |
| 1544 | CTTGCAGTTTATGCTGACCCGTCC |
| 1545 | TGCCTCCAAATTACTTACCGCCGT |
| 1546 | CCCGTATGCGGAAGCTATGGGCTA |
| 1547 | TCGTTCAACCCCACACTTCAGTTG |
| 1548 | CAATGTGGGGGACATTTCAAGGTT |
| 1549 | TAGCGTCGCACAAATGGCTGACCG |
| 1550 | GGTGGCTTCGTGACAATATCGGCC |
| 1551 | CAGCGGCGTCCGAAATTGGCTCTC |
| 1552 | GGCTTGCTCTCGTTTTTGATTGCA |
| 1553 | ATGCGAGGAGGACACGACCGTTCC |
| 1554 | CCTGTTCACTACGACCCACGGGAA |
| 1555 | GTGCCACGGAGTGCGACTGTTGCT |
| 1556 | ACACATCCAAGTCTGACGATGGCC |
| 1557 | CAGCCCGAAAGGAAAGCCTCCGTG |
| 1558 | AACTGAATGTAGGTGGGCCCCTGT |
| 1559 | ATTTTCGACGATAAGCTGGCCGGT |
| 1560 | TGAGGGAGAACCCGAAATCTGCTT |
| 1561 | GGCGACTACATCCCCAATTGCTTG |
| 1562 | GCAGACGCGGCCTTCCATACTTTT |
| 1563 | ACAACCACATGACGTGTAGCTGCA |
| 1564 | CTGCTGGGCGCGCAAAGCTTGTTG |
| 1565 | AAGCCTTCTTTGGCTTGCTCCGCT |
| 1566 | TACCTGCTGCCTGGAGCAAGGCAT |
| 1567 | GACGCCGCAGCCATGAGTGAGTGT |
| 1568 | AGTTGGCCGCTTATTTTGCTCACC |
| 1569 | AGGCGCACGGAGAACATTTGCCAA |
| 1570 | CCAGGCGCCTTCGACAGATCCTCA |
| 1571 | GTGTCCCCTCCAGCTAGCCAGTTT |
| 1572 | GACAACAAGCCAAGGTGACACGTC |
| 1573 | CTACACCGCTCGTGACTCGGCAAA |
| 1574 | TGGTGCCATCAAAGCACGTTGTAC |
| 1575 | ACAATGCGTGTTGCGAAACGCATA |
| 1576 | TTGTCCAGCCATTGTATTTTGCGC |
| 1577 | ACGAGAGATAGCGGACTCCTCCGA |
| 1578 | AGCTTTGTCGTCAGGCGAGCTCTT |
| 1579 | GACAGTCGGCGTGCAGTTTGTTGT |
| 1580 | AGCTAGCGACGGCCAACTCACGTA |
| 1581 | CTCCTGTTCGGGGCCGTTACTGGT |
| 1582 | ACTGACCGACGCAGTGCCACATAG |
| 1583 | AGGTAGGGTCTGGTTTGACTCGCA |
| 1584 | CCTCCATTTTAGCGCGTTGCCAAT |
| 1585 | TTCTTAGGATCCGCGCACTCTTGG |
| 1586 | GTCGAAGGTGTCTACCGTGCGCAG |
| 1587 | GTCACTCGGCGGCCCAATCACTCG |
| 1588 | TCTCGGTCACCCGTCTTGACCCTT |
| 1589 | GCCCTCGACGAACTCATCCTGAAC |
| 1590 | TCCGGCGTACTCTGACACGGCGAT |
| 1591 | AGCCAAATGCTTTCGTGGTTCGGA |
| 1592 | ACTCCACGCCGCATGTTGCTGTGA |
| 1593 | GCTTCGAGTCGGTGGCATCTGTAT |
| 1594 | GGTCTTGGGCCATCGACTTGCTGC |
| 1595 | GGTATCGGACTGCACTAAGGGCAA |
| 1596 | AGCCCATGCGTTCCGGATGATTTG |
| 1597 | GCCAGGGTTAAAAGTGATGGGCTC |
| 1598 | GACGACGTGCTGGCTACGAAGGGG |
| 1599 | TCCTATTGACCGTGCATCGTGATC |
| 1600 | ACCCGCCTCGACTCCACAACTAAA |
| 1601 | GATGTGGATCACGACCTGCCAGTA |
| 1602 | GTGCCATTGCCACCCATAATGCGT |
| 1603 | TTAGCCTGTGCACCCAGTCAGGAG |
| 1604 | TCCGATGGGAGAGGCTGATCTCAC |
| 1605 | CACTACTGAAGTGGCCTGGCGCTG |
| 1606 | TGCGGCCATAGCGATGTGATAGAT |
| 1607 | GATTGCGCTTAACGGAGATGCACG |
| 1608 | TCACGTTTGACAACGCCAAGCATT |
| 1609 | GCATTGTTTGCTAAAGGCGGCATT |
| 1610 | AGTCGCTCTACGCGTGCAACGCTG |
| 1611 | TAGCTCCATGGAGGTCCGAAAGGG |
| 1612 | GACCGGTTGGACCTCACTGGCTTC |
| 1613 | AAGCCGGACAGTCAATGTGCGTAT |
| 1614 | TGCCTCGCTGAGTTCTTCACCGTG |
| 1615 | TCGTAGACCTTGCTTTTGGGCTCA |
| 1616 | ACCGCTATGCGCCCTACAAAGCAT |
| 1617 | TAGCGTCACCGTAGCTTGGGGCAG |
| 1618 | CTCTCAGCAACTGATGGCACCGGA |
| 1619 | AAAGGAAATGTGGTGCTGGTCGGC |
| 1620 | CCGGCTTAGATGGAGAACAAGTGC |
| 1621 | AAGTAAATCGCCTCGCCCAAACCG |
| 1622 | TGGGCTGTTCAGCCTACCGGACGT |
| 1623 | GTTTCGGTTCAGCCATGGGCCTAC |
| 1624 | GGCCAACATTTCTAGGGGAGTGCC |
| 1625 | TTCTTCGTTGGGATTGTCCTCACC |
| 1626 | TGCACATTGGGGTACGGATCTGAC |
| 1627 | GGCAGTTAGACGGCAAACTGCAGG |
| 1628 | CGCGTCAGGCTATGAATGGCTCTT |
| 1629 | GCTGAATGCAAACCTCGGAGCCAT |
| 1630 | CGCTCTGGCGGATTCATTGTTTTC |
| 1631 | TTTTCAATCAACCCTCCGGACGTA |
| 1632 | GTGGTGGAGTCTGAAGCACGACAG |
| 1633 | AAACAGGTCCGGATGATGTCTGGA |
| 1634 | GTACCGCGTGTACGCCACCGTTAG |
| 1635 | TCCAACCTACATTTGCGGAAGGAA |
| 1636 | GACGTACCGTCGTCCCGTGAGTTG |
| 1637 | GGCAATCCTACAACCGACGCTGAT |
| 1638 | GGCGGCTGCAGGGTCTACATCGAG |
| 1639 | ATACTACGCTGCAGCTGCGCGGGC |
| 1640 | GGATCGCAATCCCTCCGATGACGA |
| 1641 | TGGCCTTGCACGGGAGCCGAATCT |
| 1642 | AGGTGCCGACGAAACGACGAATAT |
| 1643 | GCTGTTTCACCGTCGTCGTTGTTG |
| 1644 | CGGTCCCAATGTTACAACCCAGAC |
| 1645 | GCAATTCCAGCCACTTTTGACCAA |
| 1646 | ACGGGCGAAAGCTCGGTACGGATA |
| 1647 | CGACCCGACTTTTGCTTTCGAGTG |
| 1648 | AATTCAGTGTTTGCGTCATGGTCG |
| 1649 | CCTGTATGAGGTTCTGGGTCGGCT |
| 1650 | TGGCATACTTGGTGCAAACCCCCT |
| 1651 | TCGCCAGTACAGAAACATGCGGGC |
| 1652 | CCCGCTGTTGCTCTCATCGTGGAG |
| 1653 | GCCACAATCTGACCCTGGGAATCA |
| 1654 | GCTCAGTCTCGGAAGTTTCGGCTA |
| 1655 | CTTCACGGGCCAACGACGGTCGAG |
| 1656 | CGACAGTTCCGTCCGTCTTGAGGA |
| 1657 | ACGGAGACGCAGTCGAAACGTCCC |
| 1658 | CATGCATCCGATTAAGGGGATCAC |
| 1659 | ATTGCGGGAGTCCCTAGCTTTCTG |
| 1660 | GTGTGGAAGATGCAATTGGAACGG |
| 1661 | ATACAACGGTAGGTGACAGGGGCG |
| 1662 | GCCGTGGGAGTAAGGGTACAAAGG |
| 1663 | GCACGTAGGTCGGCTACTACTCGG |
| 1664 | ACTGTGATCTCTTGGGCAAAGGGC |
| 1665 | CATGCCTGAACAATCTCGCATCCC |
| 1666 | GAGCCTGGCTCCACAGCTGTGCTC |
| 1667 | CTTTCGATACCATCGTTGGCGATC |
| 1668 | CCCGGAGGTGAGGCATTGAATATG |
| 1669 | CTCATTCAGCTAAAAGCGGCTGGA |
| 1670 | GAAATGCCCTGGGGACTTTTTGCC |
| 1671 | TTTGCCTTCACAACAGACGCAGCA |
| 1672 | AAATCCCAAGACGTCGGGGCGTAT |
| 1673 | CAACGGGCGGTAGCTAAACCGTAA |
| 1674 | GGCCAACGACAATGCGAAACCTTC |
| 1675 | GACATCACGCAAAATCTCAGCGCA |
| 1676 | ACGTTCCGTCCACAACCGTATGTT |
| 1677 | GCTCATAGGTCTTCCGTAGCCCGT |
| 1678 | GAAACGAGTCTCTCGCGCCCTAGA |
| 1679 | CGGGACAGAAGCAAGTTACATCGG |
| 1680 | TGACCGCTCGATACCAGGAGGGTG |
| 1681 | CTGGCAATAAAGACCTTCCGACCA |
| 1682 | TGCGCGACGTCATGTTGGTGATTA |
| 1683 | GTTGGTTGTGGGAACACACCCGCT |
| 1684 | TGTGGGTTCGGAAACACAGGAAGT |
| 1685 | GGAAAAAACGGCAATTAGCCGAGT |
| 1686 | TGGTGCGGAGTGCCCTCTATTGGG |
| 1687 | AACCAACAGGCTGCAGCCCAGACT |
| 1688 | AAACAGATCCATCTGCACGCCAGG |
| 1689 | GGAATACCGCGGCGATTATGGCTT |
| 1690 | TACTGTTCGCGGCAAACCGTCACT |
| 1691 | GATCTCTCGTGGAGCACGTTTTCC |
| 1692 | GGCATAGCAAACCTTGACCTCCAA |
| 1693 | ATCTGGGATTCGCGAGCCAATATC |
| 1694 | CGATCAGGATATCATTTACGCCCG |
| 1695 | ACGGTACCGAAACGGTCTCAGCGT |
| 1696 | CTCCCATACCTGCGTTCTTACCGA |
| 1697 | GCACGAGAACCTAATTGTCGCACA |
| 1698 | GCCACACGATCAAGACAGCGCATG |
| 1699 | CCCGTTAACTCACGAGCGGTCAAT |
| 1700 | AGAGAAGGTCATTGCCTGTCGGTG |
| 1701 | CGGGCCCTCTTAAAGTAGAGCAGG |
| 1702 | ACATCGCGTCCGAGGGAGTTAGCG |
| 1703 | AATGCCTAATCGAGCCAGCGGATC |
| 1704 | CTCGATCTTTTTAAACCGGCGCTT |
| 1705 | CGTTCCTGGAAGGCAGGGTCTCAC |
| 1706 | CCTGTGCTTACTATCGGCGATCCA |
| 1707 | GTTAGTCGCCCTATTGGCCTGGTT |
| 1708 | CCGGTGAGATGACTGTAAATGCCA |
| 1709 | CGTGGTTTAAAACATCGCGCTTCG |
| 1710 | TAAGACGCAGAAGATGGGGTCCAC |
| 1711 | CACCACAGCTTCTTTGTTCGACCC |
| 1712 | TCGGGTCCGTACCACCACTTTTGC |
| 1713 | CCAAGCCCCGAGTACCGAAGATTT |
| 1714 | TCCGTGATATGGTCGTGGCGCGGT |
| 1715 | TGTCTGTGTCATGGCACCTCGCAT |
| 1716 | AGGACTGCACTGTGCACGTCTGAT |
| 1717 | CCATCCTCATGTACAGCGCCGCTG |
| 1718 | GTACCCGCGCCTTCCTCGACACAG |
| 1719 | ACGGGTCCTGGTCGACTAAGGCTT |
| 1720 | CGTATCGAAGGCGTGTACAACCGG |
| 1721 | TGCCCGCCCTTTATGCAACGCTCA |
| 1722 | AAACTTACGAGACGGCGGCTGCCA |
| 1723 | AAGTCTGACAAACGGAACGGGTGT |
| 1724 | TAAGCGCAGACCAAAGTATGCGGC |
| 1725 | GCAGTTTTTCAGATCCTCCGCAAA |
| 1726 | TCGGAAGCATTTACGCGATCTCAG |
| 1727 | CACAGAAACGGTTGAACGAACGCC |
| 1728 | GCATGCTCAGATGGTCGTGCTCAC |
| 1729 | AAGGATTCTCGCTTCCGGCATGAT |
| 1730 | GGTGGGGTAGCGCTGGTATGAAAA |
| 1731 | ATTATTACGGGACCGAACCAACGG |
| 1732 | GCGCGAGTGTCATGATGTTCACGT |
| 1733 | GACATTCGTGACTTGGTCGTCCGC |
| 1734 | TCATTAGTGCAGGCACCGATCAAG |
| 1735 | GAGTTGTGCGGAGTCATCGGAGTC |
| 1736 | GCCTTTACAGATTTGGCGGGCTAT |
| 1737 | ATGGCGTTTGCGAAGTCGATACAG |
| 1738 | TGCATCGGCCTCAATCAGAGAACT |
| 1739 | ACAATCATGGCAATCTGGCAAATG |
| 1740 | GACGTGGAAGAGTGCAGATCAGCA |
| 1741 | AGGGCAGGGGACGGACAGTAAGTC |
| 1742 | GCATAGGGCGAATCTAGTACGGGC |
| 1743 | TCCGGCGCATCCTCATTAGCAACT |
| 1744 | TGGCCGCTTCCACTAATATTGGAC |
| 1745 | CCGGCGGACGGCTCTTGTCAATGA |
| 1746 | CGAGCAACCCAAAAGGAAGCAGTA |
| 1747 | GCGTATGATTCGGCAATCCGCCAG |
| 1748 | AGTACCGCTACAACGCTGGTTCGC |
| 1749 | GGGCAGGCCAGGTCCACCTGAGAA |
| 1750 | CCACTTCTGTGACCGAACCGTGCT |
| 1751 | CCTGGTACCAGGCAGCAGTTGATT |
| 1752 | TTAGGGTACCGTCGAGAGACGCCA |
| 1753 | GGTTGCTTGTGCGCGTGAGGTAGT |
| 1754 | TGCTTCGACCGATGAAACTCGAAG |
| 1755 | TGCCACCCATACTATGCCCAGTGG |
| 1756 | TGTGCGGCAACGCGTGAAGACGTT |
| 1757 | TGAGAGAAGCTGGCCTCGGATCAG |
| 1758 | TATTGCGAATTOGAGTACGTGCCC |
| 1759 | CGAGAGGGGTTCCCCAGTGATCGA |
| 1760 | TGCCTGGGGTGTCGTTCTAATTCT |
| 1761 | GTGCGTCATTGTGGGTCATCCCAA |
| 1762 | AGGGCTCCCAGCATACCAACGTTG |
| 1763 | AACTAGCCGCACCTTTGTGCAGAG |
| 1764 | TTAGCCCAGCCCTTCAATGGGAAC |
| 1765 | CGGCCTCGGTTGTACGGGTAGTCT |
| 1766 | TCTTTGAGGCGCGGACCCGCATAT |
| 1767 | GATGGTTCGCCCTTGTGTCGCAGC |
| 1768 | GAGATTCAATACAGGCCGCGGGTC |
| 1769 | AGGGCGAAGGAAGGTTCCGTTTTT |
| 1770 | CTCGACCCCTGCCACTACTGGTTC |
| 1771 | TGTTCCGCGGTCTACGCATTACTG |
| 1772 | GAGACGACGTCCTACACCCGCTAA |
| 1773 | AGATTGCGACAGCGACACGTGATT |
| 1774 | GATACCGTTGGGCATTTCTCGGTA |
| 1775 | GATTGGGAGGCATTCAGCGACGGA |
| 1776 | AGGAGGAAACGAGGGCGTAGGTTC |
| 1777 | GCCAAACAACGTCTGACGCCTAGC |
| 1778 | TTTAATGCGGAAAGGATGCACGCG |
| 1779 | TTATCGGCCGTTAAAATGGGATGG |
| 1780 | CCTTGGATTCGTTCATCGCTAGCA |
| 1781 | AAGTGAACGTGCAGTGGTCTTCGA |
| 1782 | TCCTTACCCCTCGTTCAAACGCCT |
| 1783 | ATTCCTGAACCATGCATGGCCTGT |
| 1784 | AGCGAGACGCTCGATCACGAACTA |
| 1785 | GCTGGTCTGGCTCGCTGTTTAGAA |
| 1786 | CGTGCGCGGCATAAAGATAGGTCT |
| 1787 | TCTGGCACTCACATCGGACAGTCT |
| 1788 | ACCATTGGAGGACCACAGAGCTCC |
| 1789 | TCCAGGGTCGGAGTACATGGCGGG |
| 1790 | ATATGCCGTCGGATCGTACACGCA |
| 1791 | TGCTGGCGTCAACACTTCCCGATT |
| 1792 | CAGGGCGGTGCGGTGAACTAGCCA |
| 1793 | CATGGACTGCCGTACATCAGCTGG |
| 1794 | CCGGCCATACGCTGGCAAGATTAC |
| 1795 | AGCGGACACCTGTACTCTCCTCCA |
| 1796 | GGAGCCACACCAGTCGAAGATGGT |
| 1797 | CGCCACCGGAAATTGAAAAGACTG |
| 1798 | TGAAACGGATGTTGCTTCTTGACG |
| 1799 | TTGAAGCGGTGAAGAGCCTGTCCT |
| 1800 | CGAACCAAGCTGCATTGTCAGTGG |
| 1801 | GAGTCTGCGCTTGCAATCTTTGCG |
| 1802 | GCTGGGTATAGTTGCCTGGCAATG |
| 1803 | GCAGGCGTTCCATATTCGCAACCC |
| 1804 | GCGCCAACTAATACCTCCACCGCG |
| 1805 | TGGCGTTCAGTGCAACGCTGGTTA |
| 1806 | CAAAACTGACGGGTATGGGAGCGC |
| 1807 | AGGTGTCGCTGGAACCCGACTTGT |
| 1808 | CTTCCAAAAGCGCAATTGGCTTTG |
| 1809 | TCGGGCTTCTCGCAATTCTGTCAG |
| 1810 | GCCAAAAGAATGCGCTGGGTAGGT |
| 1811 | TGGTGCCCGCACCGAGAGACTGTA |
| 1812 | CGAGGCCGTAGTGGGGACTGCTCT |
| 1813 | CGATGTGCGCATAGAGGGGACTTT |
| 1814 | TGTGCAATCGGCCTTCTCAGAGCC |
| 1815 | GATCACCTGGACCGCTACCGTTTT |
| 1816 | ATGGGGAGTTAAGGACCCTGCACC |
| 1817 | CATTGTGGACAGCCAATGGTGGCT |
| 1818 | CCATCACCATGCCACGGTAAGATC |
| 1819 | GCACCCGTGTCGTTGGTTAGCAAG |
| 1820 | GGAGTGGGTTCCGCGAATTCACTG |
| 1821 | GGGGATTTCCTTTCGCAGGCTCGA |
| 1822 | CATTGATCATGTGCACTTGCACCA |
| 1823 | AGCAGCGCTGCGCTTGTTTCGGAT |
| 1824 | CGAGTAACGCGGTTGCTTTGCGAA |
| 1825 | TGGCCTGGAACATAGGTGGAACTC |
| 1826 | CGCACACCAAGCGTTTATTGAGAA |
| 1827 | TCACCTTCACAGTGGGCATACAGC |
| 1828 | CAAATATCCCTGAGCCCTCGAGCT |
| 1829 | GGGAGCTGGTGAGCAGATGTAACG |
| 1830 | AGGATTGCTTTTGCGTTATGCGGA |
| 1831 | ATCGTTTGGGCGCTACGCAATTGT |
| 1832 | CCGATTTGTCCCAAATGCAACGTT |
| 1833 | AAGGGTCAAGCTCATGGAGCGGAA |
| 1834 | TCTGACGTCGTTCAAGGGCTCGCT |
| 1835 | CGCACCACTCCGAGGTATTTGTCT |
| 1836 | AAGGGGTGAAAAAGGAGAAGCCGA |
| 1837 | AAACCACGCAAATGGCGATACCAT |
| 1838 | CAGAAGGGATGACGCCTTAAGTCG |
| 1839 | CATGACGAGAGCGGACCTGAAGTG |
| 1840 | CTGGACATGTTTGTTTCGCCACTG |
| 1841 | AAGACCGACTCTCGTCGTTTGCAC |
| 1842 | GCGCGATTACATACCGTTTCCGTA |
| 1843 | CACTGACCGGACCCAACCTAACAT |
| 1844 | AGTGCAAGTCTAGACACGCCCGAG |
| 1845 | GGTTGGTGCGAGATCCTGGACTGT |
| 1846 | GGTCGTCCCGAAACGTAAACGAGG |
| 1847 | GACTAGTACGATCACGGGGCGGGT |
| 1848 | CCGACCTGACCCTGTGTACAGGTT |
| 1849 | TGCTCACTGCCCACACTGTTATGG |
| 1850 | CGAGGAAACACATTTCTTCGGGCC |
| 1851 | TGGCACCGGGTGGATTCTTGTCTA |
| 1852 | GAGGCACGGTGATAGTGGTTGTGC |
| 1853 | ATGCAGATGGATCTTTTTCGACGC |
| 1854 | TGCGATAGCCAAAGAGTCGAGGAC |
| 1855 | ATGGCGTGTCAGCGAACTGCCTGG |
| 1856 | CAATGCAGCTCGGAAGTCAGGTCG |
| 1857 | AGGATCAGTGCACATGTCCCCTCA |
| 1858 | CACATCTTGGCTGTCACCCGAGAA |
| 1859 | CGCATTATCACCTCAATGCCAGTG |
| 1860 | ACATCCGCAGACTCCCTATAGCCC |
| 1861 | GTGAACCCGAACGAGGGGAGTCTC |
| 1862 | GCGTAGGGAATTTGCCTCACGACT |
| 1863 | TTTACGCGTCGCTCGGTTGTAGTG |
| 1864 | GAGAGGCGTCTAGGCGGTTCTAGC |
| 1865 | GCATGCTGATAACGAATGCTTCCC |
| 1866 | CTGAAGCTCGTGTGCGATGAGGGA |
| 1867 | ACAACGGCATGAGGAGGCTTTTTC |
| 1868 | TTTGGAGACGCCAGTACGCGTGGT |
| 1869 | GCTATCATTTGGTGTAAGCCCGCC |
| 1870 | TCAACATCCAGGGCGGTGCTTGGT |
| 1871 | TTCGATGTAATCCCCAAAGATGCC |
| 1872 | GGACCTTCGGCAGGTTATCGCCGT |
| 1873 | AGTAAGAAGAGGCAGGCCCCACCT |
| 1874 | AACGGCTCCCCGTCGTACTGCTTA |
| 1875 | CCTATACCGTCGTGGTTCCACGTT |
| 1876 | CCGCGCAGGCGCTAATACTCAAGG |
| 1877 | AAATGGGCCAGTGAAATCCTTGGT |
| 1878 | ACGGTTTCGAATACTGCTGGGCAG |
| 1879 | CCGCTTGAGGTTCAGGTCAGAGCT |
| 1880 | ATCGTGCCCGAAGACACTTAAACG |
| 1881 | ACCTGAACCAGGGCGATTGCTTTA |
| 1882 | ACCCTATACGCTGGGCTAAGCGGG |
| 1883 | TGTTTCGCGACTAGAAGCCTTTGC |
| 1884 | GAAGTTGGCGGCTCACCCGTATTA |
| 1885 | TGGCTACACCGCTTAGGAGGAACC |
| 1886 | CCACAGTTGCGTGACTTACATCGC |
| 1887 | ACTGCCACTGCGTCTGAAGAGTGG |
| 1888 | GCGCCAGCAAATTTCGTGTGGTGT |
| 1889 | TGCCTCCGTCGAGCCGAATAGCCA |
| 1890 | GTACAAACGGGCGCTATTTCGTCC |
| 1891 | GCTTCCCTGGCTCTGAACGGAAAC |
| 1892 | CGGCTACCCAGGCAGATAAGCTGA |
| 1893 | GGTTGGACCCGACAGGGAATTTCC |
| 1894 | GGGGAATACCCGGCGTTTGTAATA |
| 1895 | TGGTTCGGTGAGGTTATGTTCGGT |
| 1896 | TCGGTAGGGTTCAGTCGCTGAGGA |
| 1897 | TTCGGAGTGTGCCGGTGCTAGTAC |
| 1898 | TCGTACTGGAATGATGGCCGGGCC |
| 1899 | TCCGTCGACCGTCCAGCGAAGTTT |
| 1900 | AGGGAATATAACAACACCGCGCAC |
| 1901 | ATGTCCCGGAAACCAGCTACCTCA |
| 1902 | ACCAGCGACTTAGATAGCCGTCCG |
| 1903 | GGAAAACCTCCTTTGCGTCAACCA |
| 1904 | ACGTGCGTGCATACCCAAGAGGAC |
| 1905 | ACGCCACTTTCCCTAGAACCAACG |
| 1906 | CGAAGTACGCAATAGTGCCACCCT |
| 1907 | GATCCCGGCGGATCACCTATCAAT |
| 1908 | AGAAAGCGACCGTTTCAGGCTAGC |
| 1909 | CGCTCCCTTTCATAGTCCTCTCCG |
| 1910 | GTGGGTGGTCATAACGACAGCAGA |
| 1911 | CTGGAGGCTGCATCGTTCGTAACA |
| 1912 | CACCATGAGTTTCGGAGCGAGGAT |
| 1913 | CAAGCTGCGTTCGATGAGAGATTG |
| 1914 | CCTGGGAGCAATGACCGCTCTGGT |
| 1915 | TCCGGCGCTCTACCAAGATGAGAC |
| 1916 | CGACCGCGTCGCGTATACTATCCG |
| 1917 | AACATTCGCTAGTGGGGTCCAACA |
| 1918 | TGTATGATCATCCGACCGAGCAGC |
| 1919 | AGTGCGCCGAGAGGGTGAATAGAC |
| 1920 | AGGCTTGTTCTGGACCAGCACCAT |
| 1921 | GGGGCCACATAAAGAATTCCGAAC |
| 1922 | TGGTGAAGATAAATCCGCATGGCA |
| 1923 | ATTTCCACCACGCTCTTGCCAAAT |
| 1924 | CGCGTAAAGCTGTCACCGATGACC |
| 1925 | TCCCCAACCGGTAACAACAGCGAC |
| 1926 | CCTCTGCTCGCCTTACACCCATGG |
| 1927 | CAAGCTGCTCCTGTGCTGAAGGGC |
| 1928 | AAACGAACGATGGTCGGTAGACCG |
| 1929 | TCAGTTCGATGGCTATTGCGCCTC |
| 1930 | GGCTCTCAACGGACGCAAATCATA |
| 1931 | AGTAGAGTGTTGCGGCTGCCGATC |
| 1932 | AGACACTAGACCGCCGTGACCTGA |
| 1933 | ACCGAGCACCGAATTTCCTTGTCC |
| 1934 | CCGTGGCCAAGATACGAACGAATT |
| 1935 | CCTCCTACAGCATCCACATGAGGG |
| 1936 | CACTCGGCAAATACGTATGCGCAT |
| 1937 | ACCGAGTTGAAGCACGAATTTGGG |
| 1938 | GACCACCTCGGAAGATCGTTCTGC |
| 1939 | TCAACTGGGCAAACGAAGAGCACA |
| 1940 | GCTTAGCCTCACACGTGCATACCA |
| 1941 | CTGCGGTCTCCAAGTACCATTTCG |
| 1942 | GTTCCGTATTACGGCGGCCATAAG |
| 1943 | ATCGACGCAACCGGATAGTCTCTG |
| 1944 | CGCAGATAAACCGGCATCTTTCAG |
| 1945 | ACCTGCCAATACGGGTCTACGGTT |
| 1946 | ACACCTGTTGCCATGCTGATCCGT |
| 1947 | AAACTGTCTACTGCGCAATTCCGC |
| 1948 | GCAACTAGCCCGTGCTAGGATCGT |
| 1949 | TCGTAGTGGTGGATTGTTGTGCGT |
| 1950 | GGCTTACTCCTCAATTGCGACACG |
| 1951 | CACGACTCCCTGCCAGATTTGATT |
| 1952 | CTTAGACGTCGGCAATGTCACGTC |
| 1953 | CTCAGAGCACAATCTGCCCTGCCT |
| 1954 | GCTAGGAAAGTCGGCATTCATGGG |
| 1955 | AAAGCCCCAAAATTCCGCCTAACC |
| 1956 | GCGCAACGCTAAGGGACTATCAAG |
| 1957 | CGTCCGCTGGGATGAGTCTCCTGC |
| 1958 | ACAGGCCTCGTGATTGGTGTGGGT |
| 1959 | CATTCTCCTTCCGGGACCACGCCT |
| 1960 | TCGGAGTTGACCAAGCTCAGTGCG |
| 1961 | ACGCGCCACTGCAATTGCAAACAC |
| 1962 | AGTTCATGGAGCCGGCGTATTGTT |
| 1963 | ACGTTTAATGCGGGGCCCGCCTAC |
| 1964 | TGAGGCTTTAGCCTACGCGCAGGT |
| 1965 | CAGCGTTATGAGCGCGGAGTTTAT |
| 1966 | GTCCACGTGACCACGGATAGTTGG |
| 1967 | GATTATGCTCCTACGCCTGCTCCG |
| 1968 | TCGTCAAGGGCATGATGTGTGGGA |
| 1969 | GATGGACCGCCAAAGACACCTTGA |
| 1970 | TACACGAGGATGGGGTCAAGCTTT |
| 1971 | ACACGCACAAAACGTTTGAAAGGC |
| 1972 | GTTATCGTGGGCCGATGGTACTGA |
| 1973 | ACATGACCGTATCCGCCTGCTTCG |
| 1974 | GAAGGCGAACCACTGAAACTACGC |
| 1975 | TGACTTTTGCAACGGGTGGAACCA |
| 1976 | TGAATTCGTAGGTTTTGGGTGCGG |
| 1977 | AGCATTTATGAAGCGGCCATTGCG |
| 1978 | TGCTCCTCGCGTTGGTACCGTGAG |
| 1979 | CGCAGCAAGAAACAGCAACTGTTG |
| 1980 | AGACGCTTGGAGTGAAAACTCGGA |
| 1981 | CATTCGTAGAATGCCCCAAATGGA |
| 1982 | CCAGAAGGTTCGGGACCCGTCGTG |
| 1983 | GAGAAGCCGGTTCTCAGAGCACAT |
| 1984 | TTGCGTTGCAAGATATCTGGCCCG |
| 1985 | GGGTTGCATGTTCAGGCAAGACGA |
| 1986 | CTCACGAAGGTGACATATCACGCC |
| 1987 | GCCCGAGATACGGGTTCAAAAAGA |
| 1988 | CATCTTCGCGCTTCTTCACTCCGC |
| 1989 | TTACACGGTAAGCGTACGGCCGCC |
| 1990 | ACCTTCGGACAATGTGGCGTTCGC |
| 1991 | TGAATGGTTCTGCTAGGCCCACAC |
| 1992 | CACGCCTGTCTGACATATGGATGC |
| 1993 | CGCCTCAACCCAATCTGAGAACGT |
| 1994 | TTACGCTTACTGCGAGCTGGGTCC |
| 1995 | GGCTTGTGGGGCAATACGCATCTT |
| 1996 | CACTCTCCTTTGGATGCGGAACAA |
| 1997 | CTTCGAAGCACTTCAGACTTGGGC |
| 1998 | GACCAGCCATCACGTAACGGCCCT |
| 1999 | AGGAACCGGATGTGGTTATGGAGC |
| 2000 | ATCCATGGGCAACTGAGCCTATGC |
| 2001 | GGAACAGCACTTGTTACCGCCCAC |
| 2002 | TGGCTCGCTTCAAGCCTGTTTGCT |
| 2003 | CAAACGTGAGGTCATGACCACCAT |
| 2004 | ACCGATGTCTTGAAGTCCGGAGGT |
| 2005 | CGAAAATGCATGATGATCTCCCCT |
| 2006 | TTTGGTATTCTCGCTGCACCGTTG |
| 2007 | GCGTACTCAACCACATTCCCGACC |
| 2008 | AGCAAACAACAGCGGTCCGAGCAT |
| 2009 | GGACTAGGAGCGGGGATAGCTGAG |
| 2010 | CCTTAACGAAAACCTGTCGACCGC |
| 2011 | CTCGATCGCATAAGCAAGAAACCG |
| 2012 | CCCGTTGTTTGGGCGACAAAAAGT |
| 2013 | CGGCGGCTCTCGCATGATCTCGTT |
| 2014 | CGGATGGAGAGGAGTCTACGTCCC |
| 2015 | ACCAAATCAGACTAGCGACTGCGG |
| 2016 | CAGAACAATATCGTGCGTCAACCG |
| 2017 | CCTTTGCGCGCTCCGAGTAAGGTA |
| 2018 | GGAAACGGCACCTATCTGTCGTGA |
| 2019 | CGACCGACAAAACCAAATGCCGCC |
| 2020 | CCAAGGGTGTGGGAGCTGAAGAGA |
| 2021 | TTAAGTGCGCATAGTCCTCGTGGG |
| 2022 | GCCTGGTGGGGTAAGTCATGATGC |
| 2023 | GAGCAGCAGATTGATGCGCTTATG |
| 2024 | TGCGCCAACTTCCGGAATATTTGC |
| 2025 | AACCCCATCATGAAATGCTCTCCG |
| 2026 | GTCCAACGGTACTGGCGTGATGTT |
| 2027 | ACTCGGCTGATCGTGAGATGGTGA |
| 2028 | ATTCGTGGGCGCATCTCGGAATGT |
| 2029 | TCCCGTCCTGTAATCCAGGGAACA |
| 2030 | CTTCGCTGCACCTACATTGCGCCA |
| 2031 | GCGTGTAGATGACTGTGCTTTGGG |
| 2032 | CTATGGTATCGAGACATCGGCGGA |
| 2033 | CCTCGTACTCCGTCGTATGCACAA |
| 2034 | TGGTGCGTCCGTAGTGCCTGCACT |
| 2035 | CGCGATCCTAGTTGAAAGCTTTGC |
| 2036 | ACGATCCAGGTGTTGGGCACTAAG |
| 2037 | CCAATCTAGGATACACCACGCCCG |
| 2038 | GATACGTGGGGTATAGGCGGGCCC |
| 2039 | CATGGAACAAACCGTCGTAGGGGA |
| 2040 | ACACTCGCGCAGTATTCGAGTCGT |
| 2041 | CTCAGTCTCGAAGGTGATCCGACC |
| 2042 | TCCCAATCCCCGTGGTATCGTCGT |
| 2043 | AATCAACGTAGTTCCGGTGGTCCG |
| 2044 | CTTAACAACCCAGGGGTTTGGGCT |
| 2045 | CCATCCTGAGAGTGACGGAGGTGC |
| 2046 | CTACCGCTGCATGGCGTTAGATTG |
| 2047 | TTATTGGTGGCGGACGGAGTGAGT |
| 2048 | TTAAGGGTGAACTCAACCGCGTGA |
| 2049 | TTTGATTGAAACGCTGCGCACTAC |
| 2050 | TCATGTGTAGGTCGCGGCCGTCAC |
| 2051 | CTCCGAACCTTCTGGGCCTCTTTT |
| 2052 | CTGTTGCCCATTGGCCCGACACTC |
| 2053 | CACGATCGCTGAGCAACACATCAC |
| 2054 | CGGATCATAAGCGTCCGCCTTCGT |
| 2055 | AGGTTAACGCAACATGTGATCCGC |
| 2056 | GGGAAAAACAGCTAAGCCTTGCGA |
| 2057 | ACTTATTGCCGGGATCCGTACACA |
| 2058 | TGCGGTCTGGAAAGGAAGGGAGGG |
| 2059 | GCTGCCACCTGGACATCGCATACA |
| 2060 | GCAGGCATGACAGTGGCGTAGTAC |
| 2061 | GCGGCCCTGATGGTTTGGCTGAGC |
| 2062 | TCCCCATTTAGTCCCCTCCATCAC |
| 2063 | GCAACACAAATGCGAGCGTAGGAG |
| 2064 | GGCGTTTGTATTCGAGCCACGTAG |
| 2065 | GGTAACGTCGCACGTGGAATTCCG |
| 2066 | ACTTCACAACGGTCCGTTGGACAC |
| 2067 | CCGAATTATAAAGCGCAAGGCACA |
| 2068 | GGACCCGATAAGACTCTGACGCCG |
| 2069 | ACCCGTTTCTCGTAGGAACCTGCT |
| 2070 | CACGTTCGACTGTATCTGGTTGCC |
| 2071 | CCTCGGATGGGCCCATGACCTTGA |
| 2072 | GGACGCCTGCTGTAGGGGTTTGAT |
| 2073 | CTCGAGCGTGGGCTAAAAGAGCAT |
| 2074 | TTTACTTCTTAGGGCGCGTTTGGG |
| 2075 | ACCACCAACATAGCGCGCACTAGT |
| 2076 | TGGTTACACGGCAGCCCGCGTAAG |
| 2077 | TTATGGTACGTTGCTGCGTGCGGG |
| 2078 | ACCGCGGATCTAACGAATCCCATT |
| 2079 | CATGATCCCGCCCTTAGGTTAAGC |
| 2080 | TACCGCTTCAAAGGGTTGCCGAAT |
| 2081 | GCACCGCGTCAATATTACCGAGGA |
| 2082 | GTGTCGCGGCTTTACAGAAGGAGA |
| 2083 | GCAAGCCATACCGCAATAAACTCG |
| 2084 | ATGAGGTCGTGCTGCGTTCACGAG |
| 2085 | CGAGACTAGTGCCGATGCAGGGTA |
| 2086 | GCCTCATCATAGACGCTGGATGCA |
| 2087 | GACAGGCGTCGGTAAGCTCTCAAG |
| 2088 | GCTACGAATCTTCCCTGTCGCCAC |
| 2089 | TTTGGCAGAACGTACCAGTGGGGT |
| 2090 | GGACAATAAGCACCGGAGAATGCG |
| 2091 | TCATGAACCTTCTGATGCCGCGAA |
| 2092 | CGCCGCATTACCTTAAAAACGTGC |
| 2093 | ACGAGTCCAACCGCCTCATTGATT |
| 2094 | GCGAAGAGTTGCTACTCTTCCGCC |
| 2095 | CGTCGGCAACAATCTTTTTCGTGA |
| 2096 | AATCCTGTGCACCCGTGAGACGCG |
| 2097 | AACCTATATGCATCAACGCGAGCC |
| 2098 | GAACTTGGCAAAACAGCCCGGAAA |
| 2099 | CTCTATGGCCGTTTGCCGTCTGCA |
| 2100 | AGTGCACCGGGTTGTGGACACAAT |
| 2101 | CCTGGCTTTTCACACGCCAAGAAA |
| 2102 | CACTCAGCGTAGCCTGAAGCCTGG |
| 2103 | GAATTATCGACCGCAGCGGTGTCG |
| 2104 | GTGACATCACATGGTGGCCGAGCG |
| 2105 | AGCACCTTGCCGAGTCACCAGTGA |
| 2106 | TAGGTTGCAGGAATGGTGGGCACC |
| 2107 | GTCCCATACGTGTGGTACGCGGAT |
| 2108 | TCGGATACTCTCGCGTGCCACGGG |
| 2109 | CAACGTTCGCCCCTAAGCCCAAAT |
| 2110 | GTTAGGTCACCGCGGCATATCCTA |
| 2111 | GTTCACCGGCCTCTACTTGGGTTT |
| 2112 | AATCCGCGTCTAGGTCATGTGGTC |
| 2113 | GCTACGCCTCTGGAGGTGGTACCC |
| 2114 | CAGGGAATGCTACAAAGGGTCCAA |
| 2115 | AAGGGTTAGCTGCCCGGTTAACAG |
| 2116 | CCTCGCAAGCGCGATATTTATGCC |
| 2117 | GCCTCCCGGTCATGGTCAAGGGAA |
| 2118 | GCTGTTGAGCGGCGACCTGTGCAC |
| 2119 | CGCTGACTTAGCTCTGATGTGCCG |
| 2120 | TTCATGGCATTCATCACGAAGGAA |
| 2121 | TAGTGTTATGCCCGCGTGTGAATG |
| 2122 | CATGTAAGGGCACGGTCGTGGGCA |
| 2123 | CAGGAAGCTCGCTCCGTGATGCAC |
| 2124 | CCTGCTGATAGCAACCTCACTGCA |
| 2125 | ACTACGAGGGGCAGGGTCTAGGCG |
| 2126 | CATAATGTGGGTGCTGACGCCGAT |
| 2127 | TAGCGAATCCACACAGAGCCGCTC |
| 2128 | TCGCGAAATCCCTAAATCCTGTGC |
| 2129 | TGGCACGAATCAAGCCACCAACTC |
| 2130 | GCGGACCGTCTTTGCTATCTGACG |
| 2131 | AGGCCCCGCCTTGTAATTGGTCAT |
| 2132 | CTGGTCCCATACGCCGCTGACTAG |
| 2133 | TGCTAACTGCGGCCCTACAGAGTC |
| 2134 | TGGTTTTATGTTCGGTAGCGTCCG |
| 2135 | AGCTCAAACTTCTCCCACGGGATG |
| 2136 | CGCGAAGATAGTGAAATCCGCATC |
| 2137 | GAGTGAAACCTCTCGCGGGTTGCA |
| 2138 | TCGAATGCTCTGCAGTGACGTCAA |
| 2139 | AGGTGGCAATGATCGACGACCCTG |
| 2140 | ACCTTAACACAGCCGACCAGGTGA |
| 2141 | GTCCGGAGCCGTGCAAAGCAATAA |
| 2142 | TCTGCCTGACTGCTACATGCTCCC |
| 2143 | CTTTTGGGGATTAGAGGCCGACAA |
| 2144 | GGCATAAAGGCTTCCGTTCCTGTC |
| 2145 | GCGGACCGTAAAGCGGGCAGATAG |
| 2146 | TTTCAAGAGTGCATCGAATCCACG |
| 2147 | CCGGCATCCCTTCTCGCTGTTGCC |
| 2148 | ACACAGAGACGCGAACGGAGTGCA |
| 2149 | AGCGGCATTCTCCCACTCGTTACT |
| 2150 | GGAGCGTACTGCGCCTCGCAAGTC |
| 2151 | AAACCCGAATGACACGGCAGATAA |
| 2152 | GGTCGGGTCCATATCCAAGTAGGG |
| 2153 | AACCAGCGGATCGATAAAACGACA |
| 2154 | GGTGTCCACCCGTTAACGCCGGTA |
| 2155 | AGCGCGACGTGGCTTGCCGTTAAA |
| 2156 | TCCCACGGCTATAGGTCCAACGAC |
| 2157 | ATCAACGAACGATGCCGTTAGGTG |
| 2158 | GAGGCTAAGCCGTATGGCCGAGGC |
| 2159 | ACGGTCCGAAATGGTTAGAGGCAC |
| 2160 | ACGCAAACCATTCCTCGAGTAGGC |
| 2161 | TTACACGCTCGCTATTGGGCCATA |
| 2162 | CTCGGCACGGGTTTAGAACGCCGG |
| 2163 | ATTCGGTAAGGTATCGGGCTAGCG |
| 2164 | AGCACACCGTTATACATGACGGCG |
| 2165 | AGTCCCTGCCGTTCGCTCATGGAA |
| 2166 | GGGCTTATGACCAGTCAGGTTGGA |
| 2167 | GGTCACCACACGAGTGCCTGGTCT |
| 2168 | TTGATCGTGTCTCCCGAAACCCTC |
| 2169 | ATTGTCGCGATCGGCATTTCTTAA |
| 2170 | GGGTCCAACGACTTCTCGCTGCTG |
| 2171 | CAAATTCCTTGGGGGCCATAGTGG |
| 2172 | CCAGAGTATCCGCCGTTAGACGGT |
| 2173 | TCCTGCAGATCATCTCGTGTCTGG |
| 2174 | TGCGGGAGATTTGAACAAGCTGTA |
| 2175 | TTAGACGCCGAGCTAGGCAACGTC |
| 2176 | TTTCGGCAGAATCTCCGATTCAAC |
| 2177 | TGGCGAGCAGACCTACAAGACAGA |
| 2178 | GGCGACAGACCGGTACATCGGCCA |
| 2179 | TCTAGACCTGCGTTTCGTGGGACC |
| 2180 | GCCGAGCGTGGTACCATACGTTCA |
| 2181 | TAATCACACCCGCTTTCTGTGGCT |
| 2182 | GGCCGGAGCCATTGGACACTTCTT |
| 2183 | CCTGTAGACCTGCATGGATCGCTG |
| 2184 | GTGTGTGTGTCTGCGTTGGGGCAC |
| 2185 | ATCGCCGTTCCCGCAAAATAAGCA |
| 2186 | TGGATCAACGGGGTAGTGAAAACG |
| 2187 | AAGCGACGATGCTTTCTTGAGCTG |
| 2188 | CACGGGCACGTGTTCTACGCTTGC |
| 2189 | ACGGGCTGGGACAAGAGCTAGAAA |
| 2190 | GGTAACTGGCTCCGCTCTCACATC |
| 2191 | ACTCTGGCTGTTGGCGAACGTGAC |
| 2192 | GACCGAGGACCAGTCCTTGCTCTC |
| 2193 | AGTAGCTCTTGCGGCCTAACGGCA |
| 2194 | TTCTTGTCCTGGGGGAGAGCAGTG |
| 2195 | TTAGCAGGGAGGTTGTCGGCTCAT |
| 2196 | TCGGGAGAGGGCCTTACCAAAAGC |
| 2197 | AGAACGTGGATTGTACGCTCCGCC |
| 2198 | CTTCACAGCCTGGAGCCACCAATG |
| 2199 | GAGATCGATGAAACGCACCAGCGG |
| 2200 | GGGTCCAGAGTTGGTGTGGGATAA |
| 2201 | CCGTCCACCCCAGATAGGAATCAC |
| 2202 | TGCCTCGCTTCTGTGAATCTACGA |
| 2203 | GATCACAGCGTCCGCGCATAACGG |
| 2204 | ATGACGCCTTACATGACGCACCTT |
| 2205 | GCGTGGAATAACGCCCTTAGTTCA |
| 2206 | GGTCTACCATTTCTCGCCCGACCG |
| 2207 | ACACCTCTCTGGCGTAGACGCTCA |
| 2208 | GTAGAGGTGCTCAGGACTCGTCGC |
| 2209 | GTAAGCAGGAGGCGAAGGCGCGAA |
| 2210 | TCTAAGGGCCGTTTCAATCGACCT |
| 2211 | AACCTGATTTCAGGGTCAGCCCGA |
| 2212 | GTCACGCGATTGGCCCACCTATTA |
| 2213 | ACGATGCCGCGCATGTAACCTAGT |
| 2214 | TGAGAGATGTCTCGTCAACGCCTG |
| 2215 | GCATATCTCGCGGTGACAGACGAA |
| 2216 | TATCCTGGACCCAGCCTTGGAGGA |
| 2217 | GACCCAACGTCGAAATTGTGCGAT |
| 2218 | TGAAAATCGGGGCATCTAGTTTGG |
| 2219 | CCGCGAAAAGGATTTGTGTACGCA |
| 2220 | CATTCCATTTATCCGCAGTTCGCT |
| 2221 | CCTGTCTGTCGAGCCAGCGTCTAT |
| 2222 | TCAGCGCGGCTAAACAAGTTATGC |
| 2223 | ACGCCTACGAACGACCCAAGAGAG |
| 2224 | TGCGCATCTACCATTGTGTGGATC |
| 2225 | AAGTCCGCGCTCGCTCCTGTAATA |
| 2226 | GCTGGGTCATTGCTCGAGTAACCA |
| 2227 | TGGAGCGTTCTGGCAATGACCGAC |
| 2228 | CAAGTCAATTCTTGGCCAATTCGG |
| 2229 | CGTTCATGCAAGGATCCCAGGTTA |
| 2230 | ATGCCAATAGAAGCTGGGGATGCT |
| 2231 | CCTAACTCTCCCTTGAGGCCGTTC |
| 2232 | ATCTCGGCGAAGGTTCCAAACATT |
| 2233 | GCGACAGATTACGCTGCGGTTTTC |
| 2234 | AAGCCCAGACGGCCAACACGTTAC |
| 2235 | TCAAGTTCAAATCACATCCCGTGG |
| 2236 | GATTGTCGTTCTGTCTGTGAGGCG |
| 2237 | ACCGAACTATGTTCCGGCATGGCA |
| 2238 | CGTCATCGGGTGTGCAATGCCGTT |
| 2239 | CGGACGGAGTCACGTTTGTGCACT |
| 2240 | TAAACAAGTCGTGTGCCTTTGCCG |
| 2241 | TAATTACTGGCCTGTGGAGCAGGC |
| 2242 | GGAGCGGCCCGAATGGTGCTCTTA |
| 2243 | ACTAAGCAAGGCTTGGATGTGCGT |
| 2249 | AAACTAGCTAGCCGCACCCGCAAG |
| 2250 | GTTGTTCCACCAGTGATCACGCAG |
| 2251 | GCCGCTGACAAGATGATCATCGTT |
| 2252 | CTTTCATAAAGCCAACCGATGCCC |
| 2253 | CTGACTGCATCTCGAAAGCGGGTG |
| 2254 | ATTTCTTCGGAGAATCGGCCACGT |
| 2255 | CATTTCGGGCCCTAGCTACTGCGC |
| 2256 | CCGATCCCGCACATCCGTATCCTG |
| 2257 | TATCACCGGGAGCGTCTTATCGTG |
| 2258 | TAGGGCTCGTGCACCGATTAGAGG |
| 2259 | GCGTGGCACTCGCTTGTCTAGGTA |
| 2260 | CTCAACGAACTCAAGGGCCGCTAC |
| 2261 | AGCCTGGTATCGACCAATCCTGCA |
| 2262 | TACGCGTTCTAGTTGGCCGGATCC |
| 2263 | TTTATGGGTTTGTGCCTGATGGGT |
| 2264 | GGGACCCCTAGCAACGTCACCTTA |
| 2265 | CTGCCTCCCCAGGAGTCATTGGAT |
| 2266 | AACCCCGCAAGACCAGTACCAATC |
| 2267 | GGTCACATACGCGCTAAAAAGCGC |
| 2268 | AAATGGCTCCGACCAGTTAGGGAC |
| 2269 | AACGCGGCACGCTTAAAGGTGCAT |
| 2270 | GATCGCACGCCGATTAACCTTACA |
| 2271 | CCTCCTGATTGGGAGTGCGGAATT |
| 2272 | CGGAGGGTAATAGGCTCCTCTGCG |
| 2273 | ACAAGAACTGGACATTACCGCGGG |
| 2274 | TGTCGTCTTAAAGGCCTTTGTGCG |
| 2275 | GGTGACCATGTGGCGTTTTAGCTT |
| 2276 | CACGGTTGCGCACGGTACCAGAAC |
| 2277 | CCTTTATTGTTTGGTCCCCTGCCC |
| 2278 | GTGCGCCTGCATTCTACCGTCAAT |
| 2279 | GTTTACGTTGATGGCTTGCCGCCG |
| 2280 | CCGTCGGTGGTAGGACGTGAATGT |
| 2281 | TGATCGCCCCAGAATCCCTGTGCT |
| 2282 | AAGCAGCCAAAAATCGGTTGCTTT |
| 2283 | CGACGGGACTTAGTAGCAGGGCCT |
| 2284 | CCGATTCGCGAAACGACCAAGTAG |
| 2285 | CCACCCCAACTCCAATCTTTCTCA |
| 2286 | GTGCAGTAGACGACTACCGGCGTC |
| 2287 | TTCGCCCATCGTATCAAGCAATTC |
| 2288 | GAATCGCGACTACCCGTCGGGTCA |
| 2289 | CCAGCACTCGCCATCGGTTATAAT |
| 2290 | CGAACCGTAGAACTCCGGTCGGTG |
| 2291 | GCACCATGACAGAGCCCCAGGATG |
| 2292 | TGGGCTACCGCAGAATAAGGGTGA |
| 2293 | TGGCCTGTCGTGTCGAAGGAAACA |
| 2294 | GCCTCACCGATAGCGAGCGTTTGC |
| 2295 | GTGCGCGCCGGCTAAAACGAGACA |
| 2296 | CCGCAGACGAGTTTCTTGTGACAG |
| 2297 | GTTCGCAATCGCGTGCTAGGAAGC |
| 2298 | TGTTGTACACATGCATCCGGTGAA |
| 2299 | CACTGAACACGATATAAGGGCGCG |
| 2300 | CGCGATGGTTCTTAGCAAGACGAT |
| 2301 | TACACCAAGGAAGAAATGGGGACG |
| 2302 | CGTGCCTTGCGTTTTAGGTGCAGC |
| 2303 | GTCGTTTGTCTGGGCATTAACGGC |
| 2304 | CAGGCTCTCGTTCGGTACAAACGT |
| 2305 | CGGACACTGTTTCACCAGAACCCA |
| 2306 | TACCCATGATGCGGAAGAAGCGTA |
| 2307 | CTGTCCTTAAGCGGATGAGAACCG |
| 2308 | CGGGAGATGAGAACGGTTTTGTGC |
| 2309 | TAGATCGCGACTGTACTCAGGCCG |
| 2310 | TAAAACAGTTCGCGCGACTGTCGT |
| 2311 | CGAGGAGCTCCACATAAGCCCAAT |
| 2312 | TGGCTAGGGATGGGGAATCATCTT |
| 2313 | AGGATTGGGTGCCTGGATGCATTG |
| 2314 | TGTATCTACCGGCCTGAAGCAGGT |
| 2315 | TCCCTACGCGCATGACTCGCTTAC |
| 2316 | TGGTCGATCACCTGTGACAGACGC |
| 2317 | TGGGGGTAGTCCATGCATCAATTG |
| 2318 | CCCTGCCAGGATTACTATTCCGGA |
| 2319 | TCCCGCACGGGGAATTTAAGTAGA |
| 2320 | GTGATGTGCAGGAACTTCTGTCGC |
| 2321 | ATTTAGGCATGCATGCGCTTCTCA |
| 2322 | TTCGGCGCTAGTGGACGCCGTCAA |
| 2323 | GAGCTTCATCTCATCAGTTCCGCG |
| 2324 | GACAACTCCACTGCTCCAATCGCA |
| 2325 | GGCCAAGGATGGACCTTACGATGG |
| 2326 | GGTTCCGGAATTTGTCACCGCTTC |
| 2327 | GCGCTGGATAGTCTGCGAGAAGCC |
| 2328 | TGAGTCCAGTGCTGCCACCATGAA |
| 2329 | TTGAATTGGGTGTCGGAGCGTTCT |
| 2330 | CGGCGGGCAGACAATGCTTTGAAC |
| 2331 | GGGTCTGTCAAAGAGGGTGTCTGG |
| 2332 | CTTTGTGCAAGACGAAGCACCCTT |
| 2333 | ATCGAATTCCGAGGAGGTCTCCAT |
| 2334 | TCCGACCCTCAGAGTCGACTCATT |
| 2335 | ATCAACGGCCACCTCCTCGCCGAG |
| 2336 | AGCCACGGAATAATTCCGTCCACC |
| 2337 | GATCGCTTGCGTATCGCAAAGACT |
| 2338 | TCCACGCCTTACCATCAACTGCAA |
| 2339 | GCCAAGCGATAGGCCAGAACTCAG |
| 2340 | AGCGTGTGGGTCATTTTAGCACGA |
| 2341 | GTTATGCGCGGCTTACGAGTTCGA |
| 2342 | TCTGTCCACGTAACTTGCCTGCAG |
| 2343 | TCGGCAGCCAATGATCATACCTCT |
| 2344 | TAAGCCCGATCCGGTCCTGTGTTT |
| 2345 | ACATGGCAGACTAACAGGCCTCGC |
| 2346 | CATGGCTGCACTCTAAGTCGAACG |
| 2347 | TCTTCAACCCACGCGGAACGATTG |
| 2348 | CTCGTGTCTCCAGAGGATTGTCCC |
| 2349 | TGAAGGCATCAACCCAGAGGATTT |
| 2350 | ACAGCTCGAAGGCAGCCACATTGG |
| 2351 | ACAACGAGTACCGCGACAGAAGGG |
| 2352 | ATAACCGAAAAACCAGCCTGCGAT |
| 2353 | ACAACTCAGCACTTTCGACGTCCA |
| 2354 | CGGGTTACTGGGTATCACCAATGC |
| 2355 | CATCGGTTATCGCTGCACGCGCGT |
| 2356 | GAAGGAATCCCGGATAGTCCGTGG |
| 2357 | GCATGGTCTCAGCCAAAGAACCTG |
| 2358 | AGCCTGCGACGTTTCCCGACAGAC |
| 2359 | AAGAAAGGCGCACGGGATCGATAT |
| 2360 | TGTCGCGAAGCCAACTTTCAGTAA |
| 2361 | GCGGCATGCAAGGTAGGTCTGGAT |
| 2362 | GGTGGCCATCTCCTCGAATTGCAT |
| 2363 | GCGTGCATAAGTTGCACATTGTGC |
| 2364 | TTGAGGTAGCGTTTTCGCGCATAT |
| 2365 | ATCCCACTTGTGAGAGGGCGCATT |
| 2366 | CGGTCAGCGAGCAGACATCAACCT |
| 2367 | GCGTATCTTCGGGTCGAACACTTG |
| 2368 | ATGCCATTGAACTCGCACTTTGCG |
| 2369 | CGATTCCCATCATAATGTGGGTCC |
| 2370 | CAATTTGGATAATCCAGCCACGCC |
| 2371 | CGGCTTACCCTATGATTCCGTGCA |
| 2372 | GGTGGACCATGCGCTGTGGTATGA |
| 2373 | TATTTGTCGAAGATCGCAAGCGCC |
| 2374 | GTCAGTGGGTTTTGAGAGCCCGCA |
| 2375 | AGGGGGTCGGGAAATCTGACAAAA |
| 2376 | TGCTTGCTATCCGAAAAAAGCAGG |
| 2377 | TTATCGGATCAAATTCGGCTTCGG |
| 2378 | TGCAGCAACGAGTTACCCGGACTT |
| 2379 | TATACATGTCCGGAGGGGCACCCA |
| 2380 | TGCAAAACCGGAGGATGAACCCTT |
| 2381 | TCGGTCTAATGTCCACGCAGACAC |
| 2382 | ATGTGTTTGCCACGCGCTCCTATT |
| 2383 | TGGCGAGGCACGGCTCTAATTCGG |
| 2384 | GCGACGACCCGAGCGACTTTTACA |
| 2385 | CTCAGAGAGTCTATCCGGCGCCCT |
| 2386 | GGAACATCTCCTGGGTCCCTCAGA |
| 2387 | GCAACGCAGGGAAGTACTTAGCGA |
| 2388 | TGACTTGGGCGGACAAAGAAACGC |
| 2389 | AGATCATCGGGACGCTTCATGCTA |
| 2390 | CCCTTCTGACCGCTAAGGCCATAA |
| 2391 | CGTGAGCCGTGGGGTGTCTCTGTA |
| 2392 | TACCTTGGTCGTCTCCGCTTTTGT |
| 2393 | TCGCCGCAAAATGCTACGTGAAAA |
| 2394 | GAGTGACCTAATGGCTGCCCGACT |
| 2395 | AAAGGAACTTGGCCAACCCTATGG |
| 2396 | TGTTTTCGCACTCCACCTAATCGC |
| 2397 | CAATGGGTTTCATAAGGGCAGGCA |
| 2398 | GCCTAACACACAAGGGTCCCTCTG |
| 2399 | CGTCATGCGGTCCGAGGATCGATC |
| 2400 | CCACACGGGCACGGAGTAATATCT |
| 2401 | CATCAGACATAGGTCGCGTGCCGA |
| 2402 | AGATGAAACCAAGGGAGGACGCAG |
| 2403 | GGCTACCCATAGGCTCAGCAGCAC |
| 2404 | GGCTTGTGAGGGTGTGTTCTCGAC |
| 2405 | TGTGTTACGGCGAATGCAACAGTC |
| 2406 | CGATAACAGGTCGCGCCGTTACTA |
| 2407 | TGATAAAGTGAGGCTCCAGCGCGA |
| 2408 | AATTGTGCACGGATCTGCACGGCG |
| 2409 | GCCGATACTGAGCATTTCACTGCC |
| 2410 | GCAATGTACTGTCACCAGTGGCGA |
| 2411 | GGCATATCGGTAACACTTGGTCGG |
| 2412 | GGGTCTCAAACCAGCGTGGCCGCT |
| 2413 | GTCTCCGGGACCATTGAGCTGGAG |
| 2414 | GGCCTTCGGCATTCAGACGGGTTG |
| 2415 | CGTGATAGGCCACAGCGCTCAATT |
| 2416 | GGCAGGCCCGCGAGGATGATTAAC |
| 2417 | CGGGTATGGTTGATAACAGCGTGG |
| 2418 | ACGACGTCCTTGGGACCGTA1TGT |
| 2419 | CTGATATCGAGCCTGAGCCTTTCG |
| 2420 | TCCCATTGGCCTGTATGCTGGCCT |
| 2421 | GTGTCGTCGATTGTTTCATCGACG |
| 2422 | CGAAAGCCAGTAGCCGATTGCGTG |
| 2423 | GGTTCGGCTTATTCCACTGCGACA |
| 2424 | AGCGAGGGCTAACTTTTTAACGCG |
| 2425 | CGGCGCTGATGACGGGACTCGATT |
| 2426 | TCACAGTGCTCGGCGTAAGGACTA |
| 2427 | CCCATTACGAGCACACACCATGGC |
| 2428 | GGCCGCTAATCTTTACGCATCACG |
| 2429 | ACGGCTTCCTAGTGTCCAGCCCTT |
| 2430 | CTGTCAGGTCCTACCCAATGGCTC |
| 2431 | CACAGCCCATCCCACTGAACTGCT |
| 2432 | ACAAACGATACACGCAACGCTGTG |
| 2433 | TGGCGGCCAGCTAGCAGGCGAAGT |
| 2434 | ATCTCGAAACGATGCGTGCCTAAA |
| 2435 | ATCTCGAGAACAGCGTGCGTGCGG |
| 2436 | GAAGAAATCCGCCGACATCTACGG |
| 2437 | GCGGAGCAACCTTGGCTGTTTCTA |
| 2438 | CGCGTTCCGAAGACTTGTTGTTTG |
| 2439 | TGACCTGAAGCCCATCCATAAGCA |
| 2440 | TGGTATTCATTCCGGATAAGCGGG |
| 2441 | GCGTTGCGGGTCATTGATGCAAAC |
| 2442 | ACCGCTTTCTGTGTAGAGCCCTGA |
| 2443 | CAAATAGACAATCGCAGCTTCGGG |
| 2444 | TGTCCTGACAAATCAAGGTGCAGG |
| 2445 | AAATTGCACTCGCGGAGATTTCCT |
| 2446 | TGACGCCCATTTCTATATGGTGCA |
| 2447 | TGTTCCGACAGGGCACTGCTAGAC |
| 2448 | TCGCTGGCTTGGGAAGGCCTTCGT |
| 2449 | GTGCACCTCCGTTGGCGTAGAATG |
| 2450 | CTCATTTGGGACCGATCGGGTTGC |
| 2451 | GCCAGTGTCTGTCAATGGATGGGA |
| 2452 | TTGCCCGGCAGGTTCTGTGTAATG |
| 2453 | ACCCGCGAACCGAGACGCACTTCT |
| 2454 | TCCGTGCGATTGGTCAAGGTTGAT |
| 2455 | AGGGCGTCTCGGTTGAACCTCGGT |
| 2456 | TGACCGTTCAAAGAGCAAGCCAAC |
| 2457 | ACACTCACCTGCTGTCCCTGCTGA |
| 2458 | GCGTTTAACTCCTTGGGTGGTGGT |
| 2459 | CGCCTGCGCAGGTAACTCTCCGCA |
| 2460 | AATCGAATTTCCCAGCGGCTGTTT |
| 2461 | AAGCAGGTGGGATCCTGGGGATCA |
| 2462 | AATCCCAGACTCGCTCTTCGTGCT |
| 2463 | ACGGTTATAAGGGCCGGCTGCGAC |
| 2464 | TACGAGAGCGGGCTTAGACGTCGC |
| 2465 | GCGATTTTGACCCACGGTTATCGA |
| 2466 | AGCTGTATAATTTGGATGGCGCGA |
| 2467 | TCCGCGAGTCTTAGCCGATTGAAC |
| 2468 | GGCATCAGCTCCGTAAGCCGATAG |
| 2469 | TGTTATTGGCAGTTCGAGCGACAG |
| 2470 | GCGAGCCTTTTTGCTTGGGAAGAG |
| 2471 | AGAAGAAAAGGTCAGCGTCGACGA |
| 2472 | CGGGTCGACCCTTGAAGCATAACC |
| 2473 | CTCGGTTTTCACAAACTTACCGCG |
| 2474 | GCAGTCCTATCCGGAGCCTGACAA |
| 2475 | AAGGTGCGCTATTTGTTGTCGGTC |
| 2476 | AGTGGAATCCATGCCGACACCTGA |
| 2477 | TACAGGCGTAATTCCTGCGAGGGA |
| 2478 | CCGAAGTGCGAGAAGCACGTTGTT |
| 2479 | AAGGACTGGTATGGCCGGAGCTTT |
| 2480 | GGACACCGCCAACCTCATAGTTGC |
| 2481 | AATGGTGTTCGCCTGGACTACCAC |
| 2482 | TAGGAAAGCGTACACGGGAATCCG |
| 2483 | TCTCACCCCAATGATGAGGACGTC |
| 2484 | CGTGTCCGTGTGACACTGTCCATG |
| 2485 | TCCAGG CTGTTGCGGATACGGTAG |
| 2486 | GTAGGCAAAATGGTCGCGATCAAT |
| 2487 | ATCTCCGTGGACCCGATTGTGACA |
| 2488 | GAATATGCCGTCAACGCTATGGGC |
| 2489 | TTCCGGAAGCGTTTGGTAACTTTG |
| 2490 | TTCGATAGGAATACCAGGGCCTGG |
| 2491 | GGCCATTTGAGGAGGATTATGCAA |
| 2492 | ACCTVCTGACCTGGACTTTTGGCG |
| 2493 | GACCAATCCGCAGTTGAGCAACAG |
| 2494 | TCGGCCACTCACCATGAGTGTAGG |
| 2495 | AGCGCTCACATGTTCGAAAACGGG |
| 2496 | TAACGCAAAGGCGCGATCCTCGCT |
| 2497 | TGGGTGGGCCAAATATTACTGCAA |
| 2498 | GTCCTCGAAAGGGGCATCCAAACA |
| 2499 | CCCATCTGGTGGGAGGCGTTATCA |
| 2500 | GTGCGCGGTCTGCAAACTCGCCAT |
| 2501 | TGTGTTGCCAACCCTAGGTCATCA |
| 2502 | CTGATGCTGTTCTCGTCGGTTGAC |
| 2503 | AAGCTGCAAAAGGTGAGCGTGGCA |
| 2504 | TCTGACGCGTGCTTGGGAGTCTAT |
| 2505 | GAATTACTTGGAGGCGCCGTGCAA |
| 2506 | GATTCTTCCCGACCTAGGTTGGCC |
| 2507 | CGCAGCGTATCCCATGTTGCTTGA |
| 2508 | GAGATGGAATTGTTCGCCCAAAGA |
| 2509 | GATGCCTGGATCGGTCTAGCGTCA |
| 2510 | GCAGCGACTGCTAAGCTATCTCGG |
| 2511 | AGGGCTAATTTACATCGCCTTGCC |
| 2512 | AAGTGCACATCCTCACGAAGCGAT |
| 2513 | TCAGGCAGCCGTAATTAAATGCGC |
| 2514 | CCACTGGGGAAATCGCACTGTTGG |
| 2515 | TTGTCCAAAGCCACCTACGACAGA |
| 2516 | TGGGCGGAATAGATTGGGTGTCTT |
| 2517 | TAGAATTCGCCTCTTCTAGCCGCC |
| 2518 | CATTACTTCCTGCAGATGCGATGC |
| 2519 | GGAAATGCTAGCTGGGGTAATCGC |
| 2520 | GCCGCCACTTGCGAATCTACATCT |
| 2521 | ACAATAGCGGACAGCTCGCCAGAT |
| 2522 | AGTTAGGCTCTCGGTGCGGTCCAT |
| 2523 | TGGGCCTGAGAAGCGGTTAATAGG |
| 2524 | ACGCTCTGAGCGACGCCTATCGTA |
| 2525 | CCTGGTGATCGTGTCCCAGACTCA |
| 2526 | GCGTGTCCATTCGCTTGAGGTTTC |
| 2527 | ATCCTGAACGGCGATGACCACCAC |
| 2528 | TTACGTTTCTCACCGATCAACGCC |
| 2529 | GCCGTCTTGAGTGGCTAAAAGGCA |
| 2530 | ATCTACGATGCGGCTCGAAGTGTT |
| 2531 | AACCAAGACTCGTCCCCAAACGAA |
| 2532 | AACTGCGGTGGTGGAGGCAGGTGC |
| 2533 | CCTGAGTGGTCGGGCTGGAAAAAT |
| 2534 | TGCGATCTTCTCCACCTACAGCGC |
| 2535 | AGGCGCTTAGAACCGTGAAGGCAG |
| 2536 | TGGAAAATTTTGGGAAACGCTGGA |
| 2537 | CCAGCGCCGCACCTTCTCCAATAG |
| 2538 | TAGACGGCTGGCGAATCTTACGGT |
| 2539 | TACCATACAAGAGAACGAGCCGCA |
| 2540 | GTAGCCGAGAGCAATTTTCACCGC |
| 2541 | GCAAACTCCCCTGCCCTTTAGCCT |
| 2542 | ATCCCGCTGATAACCGCCAGGATA |
| 2543 | AGTCTCAGTTCGGCGCAACGGTAG |
| 2544 | AACCTACAGTCGCCGCAATGCATT |
| 2545 | ATACACGTTTCAGCCGGCAACAAT |
| 2546 | ACGACGGGACGTGCCCTCGTTGAT |
| 2547 | AAGTCCAAACTCGAATGGGGCAGT |
| 2548 | GATTTATTGGCGCGGTAACGACCT |
| 2549 | TGTTTTCAGAGGCTACCCTGCCAT |
| 2550 | ACGGTCTCAGGGAAATGCGATCTC |
| 2551 | GACTTGAAACCGCCTATGCCCACA |
| 2552 | CGATCGGTTGTGTGCTGTCTTACC |
| 2553 | AGTAGCACAATGCCTCATTTCCGC |
| 2554 | CTCGCTATCTACGCGTCTCCGAAA |
| 2555 | AGCCCGTTACGGCATCTAGGATTC |
| 2556 | TCGCGATGGCGAGAGTTCAGAATA |
| 2557 | TTACAGGATTCCAAAACCCGCAAA |
| 2558 | CGGTACCAACGCGCGGGCATATGA |
| 2559 | TGCCAGTATTATCCGTGCCAGCCG |
| 2560 | ATTTCAGACCTCGGGACAACCTGG |
| 2561 | GAAGTGCGCGTAACTTAGGGAGCC |
| 2562 | TTGGCCAGGTCATCACTCTGCCAT |
| 2563 | ATCGGCCGGTATTAGCTGCCCTCC |
| 2564 | CGCAGGTAAGGCCGAGCAATGTTT |
| 2565 | TTGGGAACGTGCTAGGCGGCCCTC |
| 2566 | CCGCAAAAGTAGAACAGCCTGGGT |
| 2567 | CATCTCGGCACACTGGTGCTGTAT |
| 2568 | ACGCGTAAATCAACGACGTGGTCG |
| 2569 | CGTAGGTGGTAAATGTTGGCCCAG |
| 2570 | GTTGGGATGCTGCTTCACTTTGGG |
| 2571 | TTCGAGCCAGAATAAAACGGTTGG |
| 2572 | AGAGATATTCGGCCTCGGTCGAGA |
| 2573 | CGACAAAGTTTCTCGCGAGCAACT |
| 2574 | ATTGCCGCGTCTCGTATCAAAAGA |
| 2575 | CGGAGAATGGATGCAGGTTCTTCG |
| 2576 | TATAATCATTTGCGACTCGCCCCA |
| 2577 | AATTTTCCCCGATTTGAAGAAGCG |
| 2578 | TCGCATACTTCGTCGGCGAGTATT |
| 2579 | CGTGAGCCGTTCTCATCCAAGCGG |
| 2580 | GCAGAATCGAATTGGGGTGGGTTT |
| 2581 | CTCTCGGTTTCTCAACCGAGCTCG |
| 2582 | GACCAGTTAGTGCAATGGTTGGCG |
| 2583 | TTCTCGCACAGCTAGTCAGCCGAT |
| 2584 | CCAAGTCTTGCGTGAGCGATCCTG |
| 2585 | GCGAAAGTGGCTCGTATTTCTCCA |
| 2586 | CCTCGGGACTGTCCGACTGAAAAA |
| 2587 | AGGCGAGTGTACGGCTCATCCATG |
| 2588 | GCGGCTCTGCCTACGATATTCACA |
| 2589 | TGCACCTGTCTGTAGATTTGCGGT |
| 2590 | CATAAAGCACGGACGCGACTTGAT |
| 2591 | CCCTCAACGTAGGGCGTGACTTTC |
| 2592 | GGGTCATCGTGCAGTTATGCCGTA |
| 2593 | CCCGGATAATCCTTTGTCCAGCCG |
| 2594 | TCCGATAAGCGAACTCACATGGGT |
| 2595 | CCTGCTGGTTCGGTCGTAAGCGAA |
| 2596 | GAGGCACCAATCGGTCTGAAAATG |
| 2597 | TACGAAAATGGTTGCGCCGGGTCT |
| 2598 | CCCAAAGATCGTATCACCACCCAA |
| 2599 | AATTGCCGGAAGCAGTCAGAATCG |
| 2600 | CCGAATCAGCCGTATTTGCTGGAA |
| 2601 | CCCGCTTATCTGTACTCGATCGCA |
| 2602 | TTTTGGGGATCCCTATTAGGCGCA |
| 2603 | AGTGACAGCGCTCACCACGGTCCC |
| 2604 | CCATGAGTGTTTCGGGACATCGTA |
| 2605 | GCCACATTCTGCTACCTCCGTGTT |
| 2606 | TCCTGTGCTTTGTGACGTGCTAGG |
| 2607 | GACCGCATATACACCTGATGGGCC |
| 2608 | GTAGGCCCGTCGTTAACCATCTCA |
| 2609 | CGGCTCGCGAAATGGAGTTTAGCG |
| 2610 | GCTGATCGGCTTTTCACCGCTATA |
| 2611 | TATCAAATCGTTGGCACGCGACTA |
| 2612 | TTGGCGAGGATCCCTAGGCGTACT |
| 2613 | AAGTCCTGAGGCCGTTCGGTTTCT |
| 2614 | ACTCCGGACATCTCGGCCAGAGAT |
| 2615 | CCAAGGGGAACACAGGATCGTAGA |
| 2616 | GTGGCCTAAATCCGCCTTCTCAAC |
| 2617 | CACTCCGTCTCGTCCATTAATGCG |
| 2618 | TCAAGAACCCAGTGCCGGTCAGCA |
| 2619 | GAATCAATTTTCCAGGGACGGGAC |
| 2620 | GAGAGCATACGCAATGTTCCCTCC |
| 2621 | ATCGGTGTGCTGGAGCGCCAGAGT |
| 2622 | GCCTCTCCTATGACGATGACCCAC |
| 2623 | TGGGCGCGCTTTTAAGACTACATC |
| 2624 | CGTTGGGTACCGTTCTATCAACCG |
| 2625 | GCAGTGAGCTGGGTTCAATGCTTC |
| 2626 | CATCATCCACACAGGCAGGTGTGT |
| 2627 | AGACAAAGGTCCCCATTGCGAAAT |
| 2628 | ATACTCGTCGACGAGAAGCGGAAA |
| 2629 | GCAGAATGTGTTGTCTTCGCAGCC |
| 2630 | CACCATGCCTTCATCTTGGCCTAG |
| 2631 | ACTCTTCAACGCCAGGTTAAGCCA |
| 2632 | GCGACCTGCGGCGTGTGTATTCTC |
| 2633 | TCGGTGTATGCACCCTTTCTCCAT |
| 2634 | ACCGTCGAATCTTGCGGCCAATGT |
| 2635 | TAATGCATGCTCCCGGCTCACGTT |
| 2636 | TCTGTACACACCACGTCGTGCACA |
| 2637 | CATGGGGTTGTCAGACGACACCTA |
| 2638 | AATCTGATGCTCGCTGTAGGACGG |
| 2639 | TCGAAACCGCGGGAAAGGGTAAAA |
| 2640 | CGCTAGGGCCTAGGGGCACAGACA |
| 2641 | TGGGGGACGGGCGTCTAATCCTCC |
| 2642 | AGGCATGCACCCATGCTGCCAGAG |
| 2643 | TCCCAATGGCCTGTCAAGCATAAA |
| 2644 | GAACCTGAGCCTTTGCTAGCACGA |
| 2645 | CGAATTGATAGCGTTACGGGCGAA |
| 2646 | TTGCACGCGCGCGAACGACTATTC |
| 2647 | TGCGGTGAAGCAGTCCAAGGTCAG |
| 2648 | TGAGGACCATCCAATGGATCGGTT |
| 2649 | TCGGTGATTGGTAATTTGGATCCG |
| 2650 | GCGGGCAGGTAGTTTGACTGGATG |
| 2651 | CAAGCACAAGCCCATGAAATTTCA |
| 2652 | CGGTACAGCGGATAGCCAAGGATA |
| 2653 | CCATGCTCTTCGCTGCAGCATACT |
| 2654 | CGCGGCAAAGATTAATTCCCGGCG |
| 2655 | GAAGACCCGTCCGGGTTTCCATAC |
| 2656 | CTGGCAAGGAGGATGTGGCTCGTG |
| 2657 | CTGTGCAGGGGGTGGCTCTGTTGA |
| 2658 | TTCAATAATGATCACGAGGCCCCA |
| 2659 | TGGTGATGCGAAGCCTTACCTTTG |
| 2660 | CTGCCACCATCTACGGCGCAGTCT |
| 2661 | TTTGCCCAGCTCTCGCAGAAGTTA |
| 2662 | AATTCAGACGCCACATCGACGGTC |
| 2663 | CCGTGGTCTGCCTCGATTACCTAC |
| 2664 | GGCGAGGAATTTCGGAACCTTATG |
| 2665 | ATCCGATGATCAGATACCGGCTGG |
| 2666 | CCATAGACTAGCGCCAGAGTGCCC |
| 2667 | TGTGGACCTAGAAAATTGCCAGCC |
| 2668 | GAATAATCATCGCGGTCCTCATGG |
| 2669 | GGGATTGGCTCTTGGTTGGAAGAA |
| 2670 | ATTGTGCTTCCTCGAACTGGGAAA |
| 2671 | TGCCCCACCCCGTAAGTCAATAAT |
| 2672 | TCAGGACCGACGGTGCACTTAGTG |
| 2673 | CCAGCCGTCACAGTGCAATTTCCG |
| 2674 | CTTAAAGAGGCGCGAAGCACAACA |
| 2675 | TACCGCTCGTCGCGATCACAATGA |
| 2676 | CCGAGTGCGCGAAGTGTCTATGTG |
| 2677 | GCACCAGTGCCCGATCAAAACGTA |
| 2678 | TGCAGGCTTCTCAACGGCTGGGAG |
| 2679 | CTCCGTACGTATCCCGCGTGATAC |
| 2680 | GGAAGTGCAACTTAAAGCCCCGCC |
| 2681 | CGAACCGGCAGTCGATCGTTGCAT |
| 2682 | CCGTTAGTGGTCGACAGTTCGGTT |
| 2683 | TCAGGCTACGCCCTCAGCACTACA |
| 2684 | TATACGGGCCGAGGTCCGTATTCG |
| 2685 | CCAACGTGTGACGAAGGGCCATTG |
| 2686 | CTGCTCAGCGGTGCTTGAAAGACA |
| 2687 | GGAGATTGACTTCGCGTTTCACCA |
| 2688 | ATGGTTCAGAAGGTTCGTCGGGTT |
| 2689 | GAGTGGAGCATTCTCGGCCCTCAA |
| 2690 | TGGATTGGAACCAATCCCGCACAA |
| 2691 | TGCTCTTGTGGTCACTCGAGAGGA |
| 2692 | TTGGGAGCACGGTTACCGCCTGTG |
| 2693 | CAACGCGAGCTAACGGTAGTTTCG |
| 2694 | AACGCTGAGCGCTCACCTTCACCT |
| 2695 | CCGTCGTAGATCTGGAGGCTTCAA |
| 2696 | GGATGGCATGGGCACACTGTAACC |
| 2697 | TCGCTCGTAGATATCCTTCACGCC |
| 2698 | GGAGCAATACCGCGTCCAAAACAC |
| 2699 | CGGTGTGCTTCAAATGCCAAAGGA |
| 2700 | TTGTTCAGACTTAGGCGCTGCCCA |
| 2701 | CGGCGGTACTCTTTCCACTGTCCT |
| 2702 | AAGACGATTGCCCACGTGCCAGAG |
| 2703 | AGGTGAGCGCAGGCATATTGCAGT |
| 2704 | CTCGGGCCTGTACAGCAAAGCCGT |
| 2705 | TGCGCGCTAGTGCTGCCTATGATC |
| 2706 | CCATCCTTTGCCTTGAGGGTAAGG |
| 2707 | AACAACAGCGTAAGACGGACAGGG |
| 2708 | GAGGCGGTCGAGGCTCACAATATT |
| 2709 | CGAGGTTAGACGCCTATGACCCAC |
| 2710 | AACTTGCTATACCGGGCGCAGCAA |
| 2711 | CGCGGTGAATCGCATACACAGCGC |
| 2712 | CACCGAATCAAGCCATATGGCTCT |
| 2713 | TTCACAGCTATCCTAGGCGCTGCC |
| 2714 | AGAAGCGCGAAGTGTACCCCGCAT |
| 2715 | TGCATGGTATTTGCGTGCGATAGG |
| 2716 | GGCCGGACCTATGTGAGATGGAAA |
| 2717 | TCAACCTGAGTCCTGATCCCAAGC |
| 2718 | TGCTTACCGTTCAGGGAGGCGTGT |
| 2719 | GGAGAGTTACGCGATGAGCCACCT |
| 2720 | CGGTATGCGGTGTACAGCTTTCGT |
| 2721 | GTAAGCCGGGTCTCGTGTCGCCGT |
| 2722 | GCGTAGTGCGAACGCCCCGACCTA |
| 2723 | TCCTCGCGGCTTACGTCAAATTCG |
| 2724 | CGACGTTCAAAGCGGGAGAGGAGG |
| 2725 | CGAGGCACCCCGACATGTTGAGAT |
| 2726 | CTATTTCGTGCCGCGTCGGACAAG |
| 2727 | GGCTGCTCAGTGACGTGTCAACTG |
| 2728 | ATCACTCGTGCGTACCCGACCGTC |
| 2729 | CGAGATGTCCTATACCGTGGCGAA |
| 2730 | TCACACCGAGCCCCATAAATGAAA |
| 2731 | AGCTACGTGTCTCGAGCAAAAGCG |
| 2732 | TCAGGGCGAGTTTTTTCAGCGGCG |
| 2733 | TTCGTTCTGTCTATTTTTGCCCCG |
| 2734 | TGGTATGCCCAGGATCCAGCCTAC |
| 2735 | TCTCAGTCGTTAGGCCAATGGCGG |
| 2736 | AAAGATCACCGTGGAGCGATCGGC |
| 2737 | TAGCAGGACTTGCACTCGTGATGC |
| 2738 | TGCCCACGGTACCGTTCAAGGCTG |
| 2739 | TGAGGTGCGTCGCCCTAAGTAATG |
| 2740 | AGCAAGGGTTACAACCCGCAACCC |
| 2741 | CACAACAGCCAGTATTCGCCACAA |
| 2742 | GGCAACACCATACTCGACGAGCTC |
| 2743 | GGCTGGATTGACAATTTAGCCCCT |
| 2744 | CGTGAGAAATGCTACACGCGTCAG |
| 2745 | CGCATCTGCCCCATTTTGTTCCTT |
| 2746 | GTCGGCCTAGTCGGCAGAACGGTG |
| 2747 | TCGACACGCGTAGCAGCGTGGACA |
| 2748 | TCCCTCACCTTCCAAAAATGTGCT |
| 2749 | GGGCAAGAACATGAGAACAGACCG |
| 2750 | TCGTCCTGGTACGACTTGCGTAGA |
| 2751 | TGGCGGTTGCATGTGATGATCAAG |
| 2752 | CCTCGCGTGAGTAAAAACCGTCCG |
| 2753 | ACTTCCGCCACAGAATGCGGCCAG |
| 2754 | GTGTAGAGCTTGGGTAGCCCCGTT |
| 2755 | CGCAGCATCCGAGTTAACACACAT |
| 2756 | ATGAGCCTGGGATGATCCGCTGGT |
| 2757 | CCTGGCATAAGTGCCGACATGCTT |
| 2758 | GCGCATGAAAAACTACGACGGACG |
| 2759 | AAAGATGGGTCGATGGGAGCGTCT |
| 2760 | ATCCTGGGCACGAGCGGATTTATC |
| 2761 | TCACCGCATTTGATAGTTACGCGA |
| 2762 | TGGTGGAGCGGACTCTGGTGTTAT |
| 2763 | CACAATGAAAAAACAATGGCCCCA |
| 2764 | CCTTGCCGCGCTTGTGGTACCAAC |
| 2765 | CCGAGACCTTTGCCACACGAAAGA |
| 2766 | ACCGCGGTGTACACCTGAGCAGGC |
| 2767 | GTCGTACGCTTACCGCAGCGGAGA |
| 2768 | TCGTAATTTGACCGACACACGCAG |
| 2769 | CCTAGACGGATACCCTGAGCGGAA |
| 2770 | AAGCGACAGCAGAGGTTCAGTCGC |
| 2771 | GCGTGGACGATATCACCTGGGCGT |
| 2772 | GTCGGAGAGCCAGTGGTACGGCTT |
| 2773 | TACCCTCCGGACCAGCTGTAATGA |
| 2774 | TATCCGCACGGTATAGCAGTTGCA |
| 2775 | CATCAGTCGGGCTACCTTCAGCCT |
| 2776 | CGGATTAATGCCTTTCCTCGGAAT |
| 2777 | TTCGTCGTGCCAAGCTAATGCAAG |
| 2778 | CCACTACGGATCAGCACAGGTGTC |
| 2779 | GGCCGAGACCACCAGTAACAGGTT |
| 2780 | CGCGCGGAAGCATTGAAGTTACTA |
| 2781 | TCGGCTTACCGCTTCGTCTGACTT |
| 2782 | GACTGACGTCAAGGCAAGCAACAC |
| 2783 | AGAGGAAGGAGGGGCTGTGACAGA |
| 2784 | TTCCAATGCGAGAGATGGCAGGCT |
| 2785 | AAATGGGGTGCTTCGAATATGTCG |
| 2786 | GCTGTCGGATTATTGCACGCCTGT |
| 2787 | CCGACTTTGTTTATGTTGCTGGCG |
| 2788 | GCTGCGATATAACCCGTCCCAGAA |
| 2789 | TGAGCTGGGCGTCAACTCCGAAGA |
| 2790 | CCCAAGCATCCTAAATCTCCCTCG |
| 2791 | CGACAGCAATCCACATGCATTCTT |
| 2792 | TGAATGGTCGGGAAACCAATGCAT |
| 2793 | CTTTGCATCGAGATGCGGGGTAGC |
| 2794 | TCCATTTCCTCCGCAACTCTCAGG |
| 2795 | CCACTACGCCATCCTGACAACGAG |
| 2796 | TAGTAAGGCCAATGTACGCCGTCC |
| 2797 | GTCATGCATATGGGGCCTGTTTTC |
| 2798 | ACCGGTAGACGTTAGCGGGTTCAA |
| 2799 | TTGGTTCAAACGGCCACACGTCTC |
| 2800 | GACACAAACTGCAAGGGAGGCATG |
| 2801 | CTCGAGCGCTGTCATCATATCGGC |
| 2802 | GCGGCTAAGGCACAAGTAGACGTG |
| 2803 | ACAGCCTAAATGGCGCAAGACCGA |
| 2804 | GCCAAATGCTTGGAATTTGCTTCG |
| 2805 | CCGATGATGTAAGCCGTCGGCCCT |
| 2806 | AGGAGCAAACAAACGCCAGTGACA |
| 2807 | ACGAATTGGGTAGCCGGACTGAGA |
| 2808 | CTGTTCCAGTTCGGCAAGTGCGGC |
| 2809 | AGACAAGTCAGGAACGCGTTTCCG |
| 2810 | AGACGACGGCCAGATACGCTGCCA |
| 2811 | AGGAAGCGCTTCTTCCGGTTCTTC |
| 2812 | GATGGACGCAAACACAAGGCGATC |
| 2813 | CGCATAGCAGTCTCCGCATCTTGG |
| 2814 | TGGTTCCGGTGTGCAACAGATAAA |
| 2815 | CCGTATGCCACCTCCAGAACTCAA |
| 2816 | GTAAAGGAACCCCTCGGGAATCCT |
| 2817 | GCCTGATGCTCGTTAAAATTGCGT |
| 2818 | TCGCACTTGGACCATGAGATCTGA |
| 2819 | TTCTCAGGCTGGGCAAGAGTCTGT |
| 2820 | CGGACCTGGGGATGCTGGGATTAC |
| 2821 | TCGAGCCGATAGGGTTGGCATTGC |
| 2822 | TACGTGTGTCCCACACACGTCGTA |
| 2823 | TGTGAAATTCGCGTTTCGCATCTT |
| 2824 | TTGCAATGCTCCAAAAAAACTGCC |
| 2825 | TCTCATCATGGCTGTGGCTTTGAC |
| 2826 | ATTACACCGCTTGGTTTGGAGTGG |
| 2827 | GCCGTGCAATGCACAGAGTTCAAG |
| 2828 | GAGATCAGACCGTGTCGGATGCTG |
| 2829 | CCACCTATCTTGATGCGACCTGGA |
| 2830 | CCGATCGCCGTTTATGTCTACGGC |
| 2831 | GAAAATCACGGTAAGGCACGTTCG |
| 2832 | GATTCTCGCTTCCCAACGAGCATA |
| 2833 | CCAGAGCAGCATTCCACAATGGTG |
| 2834 | TGTGAAATGTGGCAGTCTCAGGGA |
| 2835 | CGATCCTGCGTGCCTCATCCAGGC |
| 2836 | CCCTCAAGTGGGCGAGGGTTTTCA |
| 2837 | TCGCCTCCGCCTCGTGTGTAGAAG |
| 2838 | TTCGCTTTCAGCTCATTGGAACGA |
| 2839 | TGTAATCTGAACAAGCGGACCCCT |
| 2840 | TGGAATCTTTCTTGAGCGCCGTGA |
| 2841 | GGCTTTCATCTTTAACCGCTCGGT |
| 2842 | TGATCCGAGCCATTCCTAATCACC |
| 2843 | TGGTAGGCGTGATGTCCTACGCAA |
| 2844 | AGGCATCGGTAAGAAGGCCCTATG |
| 2845 | CGCCGCGAGACGATCCTTATTATT |
| 2846 | ACATGGACGAAATTACGCCCGTCA |
| 2847 | ACAGAAAGGTGGGGAGCCTAGCGT |
| 2848 | AGGCTTGCGAACATGGGTAGTGAC |
| 2849 | GCGTGGGCCTTGCTCCTGTTTAAC |
| 2850 | GAATACAGAGCGTCCGATGTGCCC |
| 2851 | GCGACTCTGTAGGGAGCGCGATAT |
| 2852 | GGTGCACTCATATGCGTCGCATCG |
| 2853 | CTGTCCCACGGGGAAACCTTACTT |
| 2854 | TGGCTTACTGTCGCAATCTAGGCC |
| 2855 | GCACTCAGTTTCCGGTATCCCATG |
| 2856 | GTGAGGTTCACGTAAGGCACAGCG |
| 2857 | GTAACGCCTTTGTCCCCAGCGTAT |
| 2858 | GCATTGATATGGTCGGTCTCGCCT |
| 2859 | GTGGGTTTAAGTGACAACGGACGC |
| 2860 | CAAAACCCTGCCGAAGATGTTGGT |
| 2861 | TCCGAGGAGACTGAACCTGCTACC |
| 2862 | CGGGGAAGAACGGATTCGCTAAAT |
| 2863 | TGGTTAGCTTATGTCGGAGCCACC |
| 2864 | ACGCGTCGATGAACTAAGGCTCGC |
| 2865 | TTCTCCTGACGAGTACGCAGTGGG |
| 2866 | TCCGCGGTTGCCGGTTTGTTAGGA |
| 2867 | TGGCGCATCTTTCAGGGGATGATG |
| 2868 | TCTTTGGTCCTTGGTGTTTACGCG |
| 2869 | GAGAACTCCCGCTACAAAGGAGCC |
| 2870 | TTAACGTGGGAACCGTTGGTGAAT |
| 2871 | GGGACACCATCCTTGGGTTTGTTA |
| 2872 | CAACAAACCGCCTTGGGAAGTGAC |
| 2873 | TTGAAGGCCACCGATACTGATCGC |
| 2874 | TCGTAATAGAACTGCGCCCAATGC |
| 2875 | GGCACGTTGCCCAAGTTGGATCCA |
| 2876 | ACATAGCTTGGCCGGACACCCACC |
| 2877 | CTTGCCGCCTTGCGAGTGGCTAAA |
| 2878 | AGTTCCGCGTCCTACTTCAACGCT |
| 2879 | AATGGCTCGCCAGATACCGCAGCC |
| 2880 | CAAAAGGCGTGTCCGAACTTTTCA |
| 2881 | CGTCCACTTAGGTGGAGATACGCC |
| 2882 | GAGCCTCTTCGTCCTGAAGACCGA |
| 2883 | AACATCAAGCGGCAATCTCCCTTC |
| 2884 | CGTCCTGACATTATTAGCGCGTGC |
| 2885 | TGTGCAGACCCTAACGACCTACGG |
| 2886 | TTAGGTCGGCCTAGACCCTCCGTA |
| 2887 | TCACATCGCTTAACTGAGCGCATT |
| 2888 | AGACCTTCCCACGCGAGATGCTAC |
| 2889 | TTCTTGCCAAAATGTGTCCAACCA |
| 2890 | CAGTTTTCATTGCAGCGAAAGCAA |
| 2891 | GTGCCGATCCCGAGACAAGTTCCG |
| 2892 | CATCCGGCCTCAGTGATTCTTACC |
| 2893 | TGCTGGAAGCCACAAACGTTACGT |
| 2894 | GAACGGCCAGGGGACAACTATCGT |
| 2895 | TCATCTAGGTCGAAGCGCAAGACA |
| 2896 | TTTGGTTACCAGCACCCATGTTCC |
| 2897 | GACAACAGTCTGTCCGCCACATCC |
| 2898 | GCCAACAGGAGATGCTTGCACCAT |
| 2899 | CTAAGGACGCATTGACCCCTGAAC |
| 2900 | GGTCGCGTAGTGAGTCAGAGGCGT |
| 2901 | TTACCTCATGAACCCTTCGCGGCG |
| 2902 | TATACAGCATCGTCGCCGGGCATA |
| 2903 | GCTTAGTGGCGTCTTCGTCGTAGG |
| 2904 | TGCACTCCGCAACCTTGTGAAATC |
| 2905 | AACCCGTCATGCCGACTCCATCTA |
| 2906 | AGCACTAGTGGCGTGCGACTTTGC |
| 2907 | TAAAAAGTGCCGCTAACCACGGAG |
| 2908 | CGCGGAATATTTGTCGTCCGATTC |
| 2909 | TTCTGCTATGCGTATGGGGGCCCG |
| 2910 | CGAACTACTGCGTCAGCCTCTCCC |
| 2911 | AGATGACGAATTAGCGGGGTTGGG |
| 2912 | AATAACAGTGGCAATGAGCGGGAA |
| 2913 | ATATGTTGATTCCCGTGCTGCACA |
| 2914 | AGAGTGGGCACCACCAGGCAGACA |
| 2915 | AGGCCTGGGTTTCTGCGTCTTAGT |
| 2916 | ATGACTTCAGGCACCTCAGCACCT |
| 2917 | CGGACGTGACAAACGGACATACCC |
| 2918 | CAAGTGTTTCGGCCCAACTCTCGA |
| 2919 | GAACCCTTATCGGGATAGGCCCAA |
| 2920 | CAGGACGATACCAAGCAGAACGCC |
| 2921 | GCGTCTTGTGATTCTGCCCTAACC |
| 2922 | AAACAACCATCAATGTCGGGTCCA |
| 2923 | TGTAAAGACCAGTTGGCGGCTCTC |
| 2924 | GCGTTTTGACTCGGTGGTCAGTCC |
| 2925 | TGTATGGAGGCACGGCAAAGTCTT |
| 2926 | TTACCTAGGTTCCCGCTGACACGC |
| 2927 | CGGCTCGTGGGAATCCTCTGAAGA |
| 2928 | CCGGCTCGGGCATTTCTTGGACCT |
| 2929 | CAACGATGGAATTGTCTCCTTGGG |
| 2930 | CGGGCTATTATCGGGATTATGGGG |
| 2931 | ACGTACCTGAAGATGCAACGGCGG |
| 2932 | CATGGTGCAGCACGCACAAGTAAC |
| 2933 | CGTCGATATGTCGGGCTATTGCCT |
| 2934 | AAATGCAGGGTTAAGAGGAGGCCC |
| 2935 | TGCAAGGACTGATTCTCCCGCTGT |
| 2936 | GTTTTCGGAACGCCGCAGAGTTCA |
| 2937 | CCCTCGATGGTTCATTGGGAAGAC |
| 2938 | CCTGTTCGCTCATAATGGTGGGGT |
| 2939 | GAAAGAACGATCGCGGAATAGCTG |
| 2940 | TCCACCTGTGTGCCTTTATCCTCA |
| 2941 | TCCTCCGTGAACCGCTGTAGCGCA |
| 2942 | GCCCCAGAGAGTCCCTGCTCCCTA |
| 2943 | TTGAGATTTTTACGGTTTCCCCGC |
| 2944 | CGATAGGACGTGGGCATGTCCCAG |
| 2945 | CCCGAACTTTGAGATCCGAGAACA |
| 2946 | TCACGCAGCTAGAGTCGCGTTACC |
| 2947 | AGATAACGCCCACTGACGACATGC |
| 2948 | ACGCTTAGAGCTCCGATGCCGAAT |
| 2949 | GGGCGATAACTTAAATTGTGCCGC |
| 2950 | AGGACGTTCATGCGTCTCTTTGCA |
| 2951 | CGGCTGGTAGAACTGTGCATCGTA |
| 2952 | TTCGAAATGTACTTCCCACGCGGA |
| 2953 | GCAGGTTGGCTGTCTTGTGGAGTC |
| 2954 | CGTTTGGTTGCTTCAAGAACCGGT |
| 2955 | CATACTTGGTTGTTGTGCCCACGC |
| 2956 | GGGGTCGGCTGAAGTGTTTTATCC |
| 2957 | GTGACGGTTGATTAACGACCGTGG |
| 2958 | CTTATGGCAGCGCCAGGGGCACTC |
| 2959 | GTTAGGGGACCCACCTCGTTTGAT |
| 2960 | CAATATAAATGCCGCGCATCGAGT |
| 2961 | TTCTTCATCAGCAGTCCCCGAGAA |
| 2962 | AGTTGCGTCCCTTGATGGCATTTT |
| 2963 | CCGACTTTCGTCCACGATTCCTCT |
| 2964 | ACTTGGCCGGACGACAGCAAAGAC |
| 2965 | CACCGCGGTAGATGTATCCCTTCC |
| 2966 | GTTAGCTTTAGCTCGGCACGCCTG |
| 2967 | GCGCATAAGAAGGTCCGCTAAAGC |
| 2968 | ACATCATCACGCCTGGCGTGACCA |
| 2969 | CCGGCGAAGTTTGGTGTGATTAGA |
| 2970 | TGGGAAGGCAACATGAAAGTCCTT |
| 2971 | TGCACCGCCAGATTGTGCTGAGTC |
| 2972 | ACATGTGAAGTGAGTGCCGTCCAA |
| 2973 | CCTCTGGAGGGGATTAGCCACGCT |
| 2974 | CAATAGCCATGTCACTGGCAACGG |
| 2975 | ACCCATGGTTCCAACGTTCTTTCG |
| 2976 | AATCTGGTCTTGGCATCCTCCAAA |
| 2977 | GTATACCGGTGCATGCTGAAGCAA |
| 2978 | AGTGTTCTGGTTCGAGTCGACCCG |
| 2979 | CGGGTATTCGACACACACGAGGAC |
| 2980 | AGTGCAACAGAGCGCTTGGTCACG |
| 2981 | TGCACCTATAGTTTGGTGCCGGTG |
| 2982 | TGCTCACGTACCAGGACACTCGAG |
| 2983 | AGTCCACACCTCGAACGACAGGCG |
| 2984 | CGCCGACCTGGTCAAAGAGCGCTA |
| 2985 | GCCTAAGGGCCTGTCGTTTTCCGA |
| 2986 | TGTGCGTGCTTATGTTCCGGTCTC |
| 2987 | CAACCGTTGGCCGTAACAAAAATC |
| 2988 | CGAGAATCAAGGCGTACCATCTCG |
| 2989 | GCGTAGGCAGCCTCCAGGGAATGG |
| 2990 | GATGGTGTTTTCGCCAAGACCAAT |
| 2991 | CAAGCTAGGGACAGAATTGCCCAC |
| 2992 | TAAATAGGCGAAACCGTTCGTGGC |
| 2993 | TCAAGACCCGCAATGTGTTCATGT |
| 2994 | GCGGCTGGTAGACTCTTTGCACAA |
| 2995 | CAGGCGTAAACCTGAACCAAACGG |
| 2996 | GCCGATCTGTGCTGAGGTTCATCA |
| 2997 | GATATCGCGTCGCAATATCACGCG |
| 2998 | CCCTGCACGATTAAGCCACCTGTA |
| 2999 | TGACATACAGATTTGTGTGGCCCC |
| 3000 | GTTTGCGGCCGGTATTCACGATGT |
| 3001 | TTTTACCTGGCCATTGGTGAGCTC |
| 3002 | CTCTACTCAATCAGGGTGGGAGCG |
| 3003 | GGGTTGGAGGGAGTCTTGACCATT |
| 3004 | CGAGGTCGGTAAGGAAAAGCTTGC |
| 3005 | CTTTACGCAGGCACCTCCGAGCTG |
| 3006 | CATTGTATGGCCACGTGATTGACG |
| 3007 | GTACGGTGCGAGAGCGCCTAAGCG |
| 3008 | TTCCATATGCCGAAATGGACACAA |
| 3009 | TACGCCTTCCGCTATAGCTCGTGA |
| 3010 | CTGGCCGCTCGGCTAGCCATCAAT |
| 3011 | CTGTACGCCACGCATGAAGGGTGA |
| 3012 | CTTACGCGTCCAATGACTGCCACC |
| 3013 | CACATGGTAGAACTCGATCGGCAG |
| 3014 | CGCACCGGAAACTAGTGGATGTGT |
| 3015 | ACTATGGCAACCGACACTTGGTCC |
| 3016 | CTAGTTTGCGCTACCCACCTGCAA |
| 3017 | TAGTATCGCCCGACAATAGCCTGG |
| 3018 | CCAATATTTACGGCCTGATCAGCG |
| 3019 | ATGGCTATCCCTTACTGGCTCGCC |
| 3020 | CAAAACTTGGCAGGCTTGGGACTT |
| 3021 | AATGACCGAGGCTGCAAGATTGAC |
| 3022 | ATCATCTTTCGCCACCAGACATGG |
| 3023 | CGTTATTACCGATGCACACGTTGC |
| 3024 | CACACTGGCAATCGCCTCCCTCGT |
| 3025 | AGGTTGGTAGGAAATCGGAGCGCT |
| 3026 | GCTGAACCACTGTGGTCAAGATGC |
| 3027 | CGTTGAGTACGACACGGTCGAGGT |
| 3028 | TTTTTCCGCCGCAATGTGATCTAA |
| 3029 | ACAATACCTCGACCGCTCAGCATC |
| 3030 | AGTATCCCTGCTGGCATACACGGG |
| 3031 | TCTTGGGCTCGGTAGTTCAGCACT |
| 3032 | CCCTATATCGAGCCCATAGGGCGA |
| 3033 | CACGAGTGGCATCAACGGCCTACT |
| 3034 | TGCAGGGTCCGATGTGTTCAAGTA |
| 3035 | GCTTGACCGCTGCTAACCTCGTAC |
| 3036 | TTTTGCATCTCTCCACCATCCAGA |
| 3037 | AGAATGTGCACCGGCTTCCATCTT |
| 3038 | TGTTATGACCCGCTCTGTGGCGTG |
| 3039 | GGAGCTCCTGTTTCATCGAGGCTA |
| 3040 | CATTTTGCTGTTTGGGGGTCCCAT |
| 3041 | CCCGCTCCTTCACGTGAGACGAGA |
| 3042 | GCGCTCAAGTCGATTGCCACAACC |
| 3043 | CGGTTGACGGAGACCGCAGTACTT |
| 3044 | ACTCAAGACCGGTGCACCTCCAGC |
| 3045 | TGGATGTCGAGCGTGTCTGAGTTT |
| 3046 | TTTCGTGTGCATGCAAGTAATGGC |
| 3047 | GCGGCGTTAGCTCGAGCTAACAAA |
| 3048 | GGGTATCCTGCCCGAGCAGTAATT |
| 3049 | GGCTCCGAATCTCTTGTCCGGTCT |
| 3050 | AGGATGGCCACGCCGAATCAAAGT |
| 3051 | GTGCGGGGACGTTTACATAACGAG |
| 3052 | ACTTTTGACCTGAGGCCGCTTGCA |
| 3053 | ACTCCGCTTCAATGGAGACCGTTG |
| 3054 | GATCGGAATTCGCCGCCATATTGA |
| 3055 | ATGCGTGCCCATGGAATGACTTTT |
| 3056 | CCGCATCGCACGAAGGCAGGTCAT |
| 3057 | CACCCTATGCGTCTCCAATTCCTG |
| 3058 | TGATATGCATCGCTGAGCCTCTGT |
| 3059 | AGCTTCACACGCTCACTGAACCTG |
| 3060 | AACCCGGAACCTCCTCTCACTCGG |
| 3061 | CTCGTCAAACTTGGCCGAGGAGTC |
| 3062 | GTAGCTGGCAACAGGCAATCAGGA |
| 3063 | CTTGTCACGAATATTCGCCAAGCG |
| 3064 | CAGTATCTGAAACACGGGGTGCTG |
| 3065 | GGCTAAAATGGGCGCCCACGTGTA |
| 3066 | ATGAGAGCCAAGCGCCTCAACTCC |
| 3067 | TATTGTTAGGCACCGCTTCGCGCT |
| 3068 | GGAACTAGATTGCCAGTGCTCGCC |
| 3069 | AGTCGACCCCAAGGCAACTGGGTC |
| 3070 | GGTACTGTTAGCTCGACGATGGCC |
| 3071 | CCGCAATACTTGACGGTAACAGGG |
| 3072 | AATTCCGGGTTTGAACGGTTGGAA |
| 3073 | GACACGCAATCGGGTCTATGCGAA |
| 3074 | GATTTTGGCGTCTCATTGCGTGAT |
| 3075 | TGCCATAGGGAGGAAACGCAATTA |
| 3076 | GAGGTGCCCATGTTAGTGGTGTCC |
| 3077 | GCTTTAGCGGTCATACGACCACCA |
| 3078 | CCGCTACCAACAATCCGATTAACG |
| 3079 | CATAGTGGGCTGAAACCCCAGGAA |
| 3080 | GAGGATCTGGCCACATCGAGAAAG |
| 3081 | CTCGTTTGGTACCACGTTTTGCCG |
| 3082 | AATACACGCGGCGTAAACAGACGA |
| 3083 | TGTCATGGGCCAAATGACAGTGGC |
| 3084 | ACAGCACTTCCGACCCGTGTACGA |
| 3085 | CTCCGTAAAGAGCACAGCTTTGCC |
| 3086 | ACGAACAGGTAGGGATCGGTCCTC |
| 3087 | TGGATCCACCTTACCGCGCCATCG |
| 3088 | AGTATCAAATAGCGGCGCGGCAAG |
| 3089 | GAATTACATTGTGGATGGAGGCGG |
| 3090 | CTCCTCGGGGAGTCGAGGAGTACG |
| 3091 | AGTGTCGAGCCAACTCCCACCAAT |
| 3092 | AAATGACATCCGTTTGGCCACAGC |
| 3093 | CGAATCATATCGCCATCGAACTGG |
| 3094 | TATAATGCACTCGCTTGGTGCGCA |
| 3095 | GCCAAGCAGATGGTAATTATGGCG |
| 3096 | CACGCGGGAAGAGCACGTAGAACT |
| 3097 | TACCCGAGAATTTGGAGAACAGCG |
| 3098 | TGACGGCAAACTGTGGCATCTATC |
| 3099 | CACAGTGTTCCAGCCCTTGACGAT |
| 3100 | TACCCGCCCACACATGAAAGTTGG |
| 3101 | TGGCATATTTAAGATTCGGCGACG |
| 3102 | ACTGAAAAAAGAACGGGTAGCGGG |
| 3103 | TCTGACCGCAATAGGTGGTCATTG |
| 3104 | ACTTTTTGGCGGGCCCTCTCTCGT |
| 3105 | CTGCCCAGATCATTGCGCGATCCG |
| 3106 | CGGAGGTTAAATGCTTTAACCGGC |
| 3107 | AGGCGTCTCCAAACGTCCTTCTGT |
| 3108 | AGATGCTATCCTGAGTGGGCCTGC |
| 3109 | ACAGGGTGAAGAGACCGTGGGATG |
| 3110 | GACTGTCTAACGGACGACACGACG |
| 3111 | AGCTGTTAGGACCCGACAACCGGT |
| 3112 | TTGCGTAGTGTGGGCATTTCCTCT |
| 3113 | ATGCGCGCTTCTTTCCTTGATGTA |
| 3114 | TTAAGGGCGTCCGCGTCTATTCAG |
| 3115 | ACCTTTAAACTTGTACCGCGGCCC |
| 3116 | AGGGATGCAGAGGCACCACATGTT |
| 3117 | CGGTTCGACGTATGAGCATCCGCA |
| 3118 | CAGGGCGATAGTCACATGGAGGTT |
| 3119 | GCTTGACTGCCCCGTTTCATATGT |
| 3120 | CGAAGGGGTTGTGCAATTACCCGA |
| 3121 | AAAACGCACCGCAATGACAAAATT |
| 3122 | ATTCCTGGACAAGACCCTCAACCG |
| 3123 | CCTACCTGCCTGCTAGCGGTGAGG |
| 3124 | GCTCGTAAATGGGGAGGAATTGGA |
| 3125 | ACATGAAAACAGGCTCAATTGGGG |
| 3126 | GTTCCGCACATGGATTGAGGTCTC |
| 3127 | GGCACCCAATACCACGAAGAAGAA |
| 3128 | AGGGGCATTTCGAACTCCATCTTT |
| 3129 | CATCATCACAAAGGAACGTCGGTG |
| 3130 | TAAAGACCCACCGTCAGCAGCAGC |
| 3131 | CCCCAGGCGTAATGCACCACATAG |
| 3132 | GCAGGTCGAACGCTAGTGGTTGAA |
| 3133 | GGAACTTAGGAGTTCACGTCGCCA |
| 3134 | GCAGATACGGCTAGCTGAGGTGGC |
| 3135 | CACAGGCCTAGAGCCTCGGCGTTC |
| 3136 | GTTTTGCGCGCATGAGGTTCATTA |
| 3137 | TTGCGCCTGATGCCAGCAGTACTA |
| 3138 | GATATCAGGCTTTCCCACTGCCGC |
| 3139 | TGCGCGGAGACGGAGATCTATGAA |
| 3140 | CATTGGTGTTGGCTGAGAGTGGAC |
| 3141 | GTCGGCACTTGGGCACCATTAATA |
| 3142 | ATCGATCGGTGTCTCACCACGGAG |
| 3143 | CGTAGCCTTCCACCGTGTCGATAG |
| 3144 | CGCTCTCCGTCTGAGGAAAAGGGG |
| 3145 | TCGCCCCAGCCAAGGATATATTGC |
| 3146 | TCTCTTGCAAGGAACTCTGCCGTC |
| 3147 | GTCCTGGACAGACGGAGGGTGTTA |
| 3148 | GCCAAATTAAGCGGGCTCGTAATC |
| 3149 | CCATTTGTTGACCGATGGGAGGGG |
| 3150 | TGGTCAAAAGAGCACGATCCAGGA |
| 3151 | CGCTACTAAGACGCCCCTGTCCAC |
| 3152 | CATACCTCCCGCTTGGATTCACTG |
| 3153 | CCGCGGAAGGAATGTCATCTACAA |
| 3154 | CACGGGACATTCATTCACAGGACG |
| 3155 | ACTAGTGAGGCGTGAGGCGGGCGT |
| 3156 | AGGAGTCACCCACTCCGCACAAAA |
| 3157 | TCATGACAGCGCACCCCATACCAT |
| 3158 | GGTAGGGGACTATCGATCGTGCTG |
| 3159 | ATGTCTCACTACCGCACGTAGCGG |
| 3160 | TACTGCTCCGGTCTTCCGCAGCTT |
| 3161 | ACGGAGGAGCGACTCGTTCGCTGC |
| 3162 | GAAGTCTGTCGCCGGTGGACGGAC |
| 3163 | CCGTAACGTGTATTCGGACGAGCG |
| 3164 | CGTGGAAGCGACTTAACCAATCGT |
| 3165 | GGCATGGGCTATGCCTCACACTAG |
| 3166 | GGGTCGTATTTCAGCATCGTTCGT |
| 3167 | AATGGTCGCGCAAACCGTAAGAAT |
| 3168 | CTGGATTCGGTACGTCCAACGTTT |
| 3169 | CGCAAAAACACCCGTAGCCAAGAA |
| 3170 | TATGGATACGCTTTTGGACTGGGC |
| 3171 | GCTTCAAACGCGCTTCACGCTGGT |
| 3172 | TACAGCCCGCTCTACCTCGCCACC |
| 3173 | TCAACCGATGTCAAAATGCACGTT |
| 3174 | AGCTCTCTCCGAAGTAGGGCGGTA |
| 3175 | ACGCACACATGGAGACTTGGCTCC |
| 3176 | TTCTTGAAAGCTAGTGGGGCGCTA |
| 3177 | CAATCACGGCTGGGCTATTCTGTG |
| 3178 | GTGGCGACCCGTCGGTGAAAGAGT |
| 3179 | CGTCGAATGCCGAACCAGTTAAGT |
| 3180 | TGCGTATTTGCATGCTCACAGCTG |
| 3181 | CGCAGTTGGTTTGTGCACGGCTGC |
| 3182 | GTTTTTCCGTGAAAACTGGCATCG |
| 3183 | ACAGGTTCCTCCACCACGATTTGA |
| 3184 | CTAGCGCGCTTTTAGGTCCTTGCG |
| 3185 | CAAAATCAAAGGGATCAACCGGTG |
| 3186 | AACGTAACCCCAGTGAGTCAGGCA |
| 3187 | TCAACCGGTGCACTTTAGAACGCC |
| 3188 | ATCGCAAAGTTGCAGGCGAATACT |
| 3189 | ATATGTCCCTGGGTGCTGCACAAC |
| 3190 | TGGCACTTTGTAGTGCTGCGGTGG |
| 3191 | ACGCACGACGTCCTTCTAAGCTCG |
| 3192 | CCCACGTGCACTATAGGGATTTCG |
| 3193 | CCGCGCTTGGTCAGTCATCCTTGC |
| 3194 | AGCGGCTCAGGGAATAACAACAGG |
| 3195 | ACAACGCGATCGGAGGCAACCAGT |
| 3196 | AGCAATTGCCTCCGTAGAAACCCA |
| 3197 | GAGTCGTGGCATCGCCTGCTATCG |
| 3198 | TCTATGCAAATACTGCGCTTGCGA |
| 3199 | TCAGCTTAAGTTACGGTGTGGCCG |
| 3200 | TCCAAGGTCGAACAGGGATCAGAA |
| 3201 | GTTAGGCTGGCGTCAATAGCGCTT |
| 3202 | GGTGTCATAAGGAAGAGGGCATCG |
| 3203 | CCGGCGGGCTAGATCAATATTTCT |
| 3204 | CTAACGTCAAGTTTTACGCCCCGA |
| 3205 | GCAGCACAGTTTTCCGATTTGCGG |
| 3206 | CGCACGCAAGGGGAGGGATGACTG |
| 3207 | CGGGGCCGAAAAGGACGTCACAAG |
| 3208 | TTCTCCAACACGGCTAACCGGTAG |
| 3209 | TTACAGCCTGGCCCGAGGTAGTTG |
| 3210 | TTTCGGGCAGCATGAGTTATCGAA |
| 3211 | CTACTGGACGCCCTGCTTCGAAGT |
| 3212 | GGTCGTCCGACGTGAAAAGACCAA |
| 3213 | GTTTTCGAGCTCTTTCTCCGCAGG |
| 3214 | GCGTGAAGGTACCCAGTGTCACAG |
| 3215 | TTTCTGAACGCTTCGACGCAACAC |
| 3216 | TGCTAATAAGCACGCCTAGCCCGT |
| 3217 | AAATTAATTGTGGTGGCTCCGGCG |
| 3218 | TTACAATCCTCGGGCTCACTGACA |
| 3219 | GCTGAAGGACAAGGCGTGGGCAAC |
| 3220 | GGGATAGGAGACCCTCGCAATGGT |
| 3221 | TTGCAGTACGTCCTTGCGCATGAA |
| 3222 | TTGATCACTGGATTGGGTGCGAAC |
| 3223 | TCTGCAGACGTTGCGAGAGATGAT |
| 3224 | AGTCTAGCAGGGATCGAAGCGGAT |
| 3225 | GGGGTCCCGCAACAACTAATGAAG |
| 3226 | CAACCTCTTATGTGGTGTGCGCGA |
| 3227 | CTCGCTGGGTTGCTGGAGTAGCAC |
| 3228 | CGTTGTATTGTGCAACGCGAAGTT |
| 3229 | GGGCTCAAAGTGCCTGAGTCGAAA |
| 3230 | CTGCTGTGCCCTCTCAGTGAGAGC |
| 3231 | CGGACGTACTGTTCGGAGTCCTCA |
| 3232 | GTATACCACCATACCGGGACCGCA |
| 3233 | CTGCTGCGAAGGGAGACACGTCCG |
| 3234 | AAAGAACGTGGAGGATCCATTGGG |
| 3235 | TCGATTGGCTGATCTCCAGCCTAC |
| 3236 | CTGCGAATTCGAAGGTTGTTACGG |
| 3237 | GCAGGAGGGTCAGGAGTACGTGAG |
| 3238 | ACCAACGGAAGGGAACTTAAGGGC |
| 3239 | ATGATGGAGGCTGCGTTTTGGTCG |
| 3240 | AAGCCCAATTTACCGCTCCGAATA |
| 3241 | CTAGGCTGTGCGGGACTAGAGGTG |
| 3242 | TGCCATCTGACCTGGTGATTGCGT |
| 3243 | GTCGTCAACTTTTATCGCGCACCT |
| 3244 | TTGAATGTAGGCTGCTGCAAGCGC |
| 3245 | CACCTATCGTGGCCTCTGTCCCAG |
| 3246 | GGAGCGCCCAGTATAATGAACGTG |
| 3247 | AATGGGGGTTCTTAGGGTGCCGTA |
| 3248 | GCCATGAGGAAAAGCACTGGGTCT |
| 3249 | TCCGGGTCGTACTGTGTATGATCG |
| 3250 | GGAGGTTATGTGCTGCTGATGACG |
| 3251 | CTTCAGCCGTGAATGGTGTGAAAG |
| 3252 | CTTCAAGGGCTTCGTCTGCTCGTG |
| 3253 | TCAGGGGTCACGCATTGGGTTTCA |
| 3254 | ACGGTCCTCGCATAATGGACCACT |
| 3255 | AGGCGTAAACGCCGGTCATAGTCT |
| 3256 | GATCTGGTCGGAAAACAGGAGCGC |
| 3257 | CCCATCGATGTTATTTCCGACGCA |
| 3258 | TGTTTCTCCGCATCAGTACCGCAT |
| 3259 | CGGACCCGGATCGACAAGTAGTCA |
| 3260 | AGCCAGAGCATGAACTGGAGCGTC |
| 3261 | TGGAGTTTACATCGGAACGCAGGG |
| 3262 | TCGACCACCGGTACGATACAATCA |
| 3263 | GCTTGTGGAATTCCGACGGTTCCA |
| 3264 | CACATCCACCCTACTGAGGCACAA |
| 3265 | GCCGGATGAATCTGCCTCGCTACA |
| 3266 | GGTTGCAATTACGCCGGGATTAAA |
| 3267 | ATTTCCTCGCAAATCGTCTGGGTG |
| 3268 | GCTCCTACGCCATGTGCACGTTTA |
| 3269 | AGGGTTGTCGAAACATGGGGGTGA |
| 3270 | ACGCGACCTGCTGTCAGCGTGGTG |
| 3271 | CGCCTAACTAGGGGAGTGAACGGA |
| 3272 | GTTGACCTCCGGATTTGCTCACGA |
| 3273 | TACCTCCGTCATTCACTCTTCCCG |
| 3274 | GGCGTTCCACATGTAATTGGGTCT |
| 3275 | CGCATCACGATCGTTAGGAGGGAG |
| 3276 | GGGCATTAAGCACGCACTTCGTCA |
| 3277 | TTTCCATAATTCGACACCACGCGG |
| 3278 | GACCATGAGATGCTTTTCTTGCGC |
| 3279 | CGCGGTCGTCCTCAGAGAATGTTG |
| 3280 | TGCTGTGACGATGGCTCCTACCCG |
| 3281 | GGCGAATGCTTCTTCGCATCAAGT |
| 3282 | AAATGCACAGCGGAACTGACCACA |
| 3283 | TATCGACCTGGAACACGATCGGTT |
| 3284 | CATTGAAGTCATGAAGCCTGGTGG |
| 3285 | CTTTCAACCGTAGTGGCTTGGGCA |
| 3286 | CCGGTAAGGTCGAATTGGAGCCTA |
| 3287 | GGATTGAAAAATCGCCGGAAGATC |
| 3288 | TGAAATTGTGAGGGAGCCTTAGCG |
| 3289 | AGCGGGATCCCAGAGTTTCGAAAA |
| 3290 | CGAGTGTCACTGGTCGGTTGCTCA |
| 3291 | GCAGCATCCGTTCCCCTATAGTGG |
| 3292 | GTATTCCTGACCGGCTGAGTGTCG |
| 3293 | GCAGCGTATGGGGTTAGCCAATGA |
| 3294 | CGCCCTGGTGGAGTTGTATGATGA |
| 3295 | AGGTAGACTGCCCGCGGCAGAGCA |
| 3296 | ATGCGTGAGGAACTGACTTCGGAC |
| 3297 | ACGGGAGAGGACATGCATTTTCAA |
| 3298 | ATTCATGCAGGAAGTCCGAGGGAA |
| 3299 | AGCTCTCTCCGAAGTAGGGCGGTA |
| 3300 | TGGCCCACATGATTGGAGCTCCAA |
| 3301 | GCCCTTTGCTTGCATTGATTGATC |
| 3302 | AGGAGATTCTTCGGCTCATCTCGC |
| 3303 | GCAGCTCCGCCAACGAACTTATAG |
| 3304 | TGGGTCAGCTTCGGCCAGGCTGAT |
| 3305 | ACGCTCAGCGTGCGCTAGATACGA |
| 3306 | GCAACGAGAGCGAACGGTTAACTC |
| 3307 | GAACACAAACAGAGGTCGTCAGCG |
| 3308 | CGTGCGTTAGCGTCGGCGTATGTT |
| 3309 | GTGCTAGCCGAAAGTAGCGTGCGA |
| 3310 | CGCGGAGGTTTGCAAGTTGTTAAC |
| 3311 | TACTGCCCGGCCTGAAATGACTTA |
| 3312 | CATGCGCACATGAGGGTCACCTTT |
| 3313 | CTCGGGTTCTGAAAGCGATGCTTC |
| 3314 | GGCACACAACGAAGGCTGATGATA |
| 3315 | GGAGGCCGAGTAACCTTGAGGGTC |
| 3316 | ATTCCTATCGCGCGTGCTTCTAGC |
| 3317 | TTGCCGGTGTGTTCGTGAGCTGTT |
| 3318 | TTATGGGAATCTACAAAGGGCCGG |
| 3319 | GGGTGATCCAAAATCCACGGAGGC |
| 3320 | GCGAGATGAGCAAATTGTATCCCG |
| 3321 | CCTGCACACATCATGTCTCAATGC |
| 3322 | GGCAGCGTAGGGATTTCCTAGGGG |
| 3323 | AGAGATTGCTCCTATGTCGGCAGC |
| 3324 | CCAATACCCTGGTGACCACTCCAA |
| 3325 | GACGTCTGTTATGTCGTCGCAAGG |
| 3326 | CCACAACGTCGAAATGACCTACCA |
| 3327 | CTTGGTGGCATGCATGCCTTGCCC |
| 3328 | TACGTTCGCCCGACGTGGAATAAA |
| 3329 | GGAAGAGAAAACCGACAGTCGCGA |
| 3330 | GACGAACAAGAATTTGGGGCAACC |
| 3331 | CGTGCCCGCGAGTTCATGGTGCTA |
| 3332 | AAGAGAAACCCTTTCCGGAGCTCA |
| 3333 | TTTTAAATCTGCCGCCCTTCCATG |
| 3334 | TCTGAAGCAATTTGGCCTCCTCAA |
| 3335 | GATGCGCAAGAGGGTATTATGGGC |
| 3336 | GTGAAAATCTCGCAACTTCCTGGC |
| 3337 | ACGGGAAGCGGTGAATTGTTGGTA |
| 3338 | GCCCTACTATTGCCTTGGCAATGA |
| 3339 | GTAAATGGCAGGAAGCGGCTCTCG |
| 3340 | AGGTGCCAAATAGTGGACTGCGGT |
| 3341 | TCGGATGGTAGGAGGCGAGATCGG |
| 3342 | GAGGTGAAGGAACAGCGACGCTAA |
| 3343 | ACCGTCGTTACCGCTCTGGTGTCG |
| 3344 | TTCCAATGTCCGACATGCTATGCC |
| 3345 | CGGCTTTATAGGTCCAACATGGCG |
| 3346 | CCGGCCTGGAAAGCAGAGTTATTG |
| 3347 | TTTATCGTTCAACGCTCACGTCCC |
| 3348 | AGACCCGCTGAACGGAGCTVGGAT |
| 3349 | ATCCATCAGGAGAAAGCTGGCTCA |
| 3350 | TTGCCAATGCGTAAATCGGTTCTC |
| 3351 | GCTTGGCAGAAGGCGTACACTAGG |
| 3352 | AGGCTCCAATGCTTTAGCCGCAAA |
| 3353 | GATACTAGGAGCGAGCCCCTTTGG |
| 3354 | GTCGTGTGCAGCCGCATATGGAGG |
| 3355 | TACCCCTGTTGCGGATAGATGTCG |
| 3356 | TAGGGTAACAGAATGAGGGGCGCT |
| 3357 | ATCGTGTCGGGGATCGAATTTGAG |
| 3358 | ATCTCTCGTGCGGTCTTGCAGAAG |
| 3359 | AGAAGCCACATGTTAGTGCGGGAG |
| 3360 | ATCTGCGTTAACTGTCCCGACTGG |
| 3361 | CGCTCACAACGAGCTTACTCATGG |
| 3362 | TCTACGCTACGATCCGTTGCATCA |
| 3363 | TTTAACACCGAAATGGGAGCGTCC |
| 3364 | ACAGGGCGTAGTAGGCCGCTTTCC |
| 3365 | GTCGACCGTGTTTGTGGGGGATAT |
| 3366 | AGAAGACCTTGGCAATCCGAGTCA |
| 3367 | TTGGGTGCTTAAAATGCGGTCTGA |
| 3368 | AGCGAAGTCGTATTGACGTGCGGT |
| 3369 | ACTTTCAGCTCCCAGTAGCACGCA |
| 3370 | GCGCATGGTGAGTCCGTATTGCCG |
| 3371 | GGGTCGTGTCAGAGGACAAACACC |
| 3372 | ACAAGAGGACCTCCGGGTGAAAAT |
| 3373 | TAGCGGGGACCTATCCGCCTCAGT |
| 3374 | GCTCTATGCCATGTCCGTGGATTC |
| 3375 | AGCTCATAATGCGCGTTGACCCCG |
| 3376 | ACAGTGGAAACGTTTCATGCCGAG |
| 3377 | GGTTTCGACGAAAAGGATGGTCGT |
| 3378 | GCGGTACGTATTCTAACCCGACGG |
| 3379 | GGTATTCGCCATGCTTGGTCTCTG |
| 3380 | GAGCCTCTCCGATTCTGGCCCAGA |
| 3381 | TGGAACGTAATACGAACGCCGAAC |
| 3382 | GGCAGAAGTGGAACTGAGCTCGAT |
| 3383 | CGGGTAGGCCTTCAGGGTACAGGT |
| 3384 | AGCGATCTTGGACGCCGGCACGAT |
| 3385 | GACCAGGTTGGTACAACGCCTTGG |
| 3386 | GATGTGCTACAGGACCGCCTACGC |
| 3387 | TGAGGCGCACTCATTAGGAGGTGT |
| 3388 | CACCTTACATCCCGAATCCGCGTA |
| 3389 | CCAAACATAAGGTGTGTCGGTCCA |
| 3390 | GCGTTTGCTAATGGTTGCGATTGC |
| 3391 | CCCTTGCCCTCAATCTGTATTGCA |
| 3392 | ATAGTCCCGTGGCGACTGTGATCC |
| 3393 | GAAGTTCCCGGCCCGAGTAACATA |
| 3394 | GGGAGCCACGACAGAGCTCCTAGG |
| 3395 | CTGACTCTTACGAAGCGCACTCGC |
| 3396 | AGGTATAGCGGGGCGTCTAGCAAA |
| 3397 | TAAGACGCATTGCTTGGACCATCC |
| 3398 | GCCTAGTAGGCCACGGCTTCATGC |
| 3399 | CGTGCCCTAGCATACAACGTTGGG |
| 3400 | GGGAATGCGGCAGTCTGTCTACCT |
| 3401 | GTTGAAATACTGGCCCCGCGGGAC |
| 3402 | CGGACAGGTGAACCCAGTCACCTT |
| 3403 | CAACAGCCCGCTCCTTGGATATAA |
| 3404 | TTAAAGGAATCAGGGGGACCCGCC |
| 3405 | CGGGTTGTAACGCTGTTGGACGAA |
| 3406 | GGTACGCAGCGGGACCAATAGAAA |
| 3407 | ACTGCAAGCCTCTTAGTTCCTGCG |
| 3408 | TCAATACCACCCAGAAACTGGGCG |
| 3409 | GGCAGTTGACACTCATCGACCATC |
| 3410 | TAGCACGGCCATAAGACGGTTGAA |
| 3411 | TCCACAATGTCAGCTCACTGCAAA |
| 3412 | CAGGCGGAGGGGTTTTACATCCTA |
| 3413 | AGGGCACTCGAAGATCCGACGGGC |
| 3414 | CGCAATGCCTTTTGCTGTGGTAAT |
| 3415 | AGAAACGCAGACGTGGCGTTTTGT |
| 3416 | TGAGCACGAATGTCGAACAGTCAA |
| 3417 | CTCGTTTCCATGGGGTAACCGACT |
| 3418 | CCTCATAGCTACGGGTGGACGACG |
| 3419 | GTACGCCGTGTATCACCCCATTCA |
| 3420 | ACCCATAGTTCGTCGATAGCGCGA |
| 3421 | TCTGCAGTGTTGCCCCTCCGACGC |
| 3422 | TGCACATGCAACTAATAGGTGCGC |
| 3423 | CAGCGCAGTGCCTTACCAATATGA |
| 3424 | TTACGCGCCGAAAACACCTGAACA |
| 3425 | CTCCCTCGCTTTATATAGGCGGCG |
| 3426 | GTCGGACCCCGAGAGTCCTGTTAA |
| 3427 | ATCGACGAACAGGGCCTCCGGCTT |
| 3428 | TGGTTTTTCACCTCCGTCCTCAAG |
| 3429 | GGAGGGGGCCAACTCCTTGACTTG |
| 3430 | TCCTGTCTCGGCCTTTGGGAACTT |
| 3431 | CAAGCCATTACCCGCTAGCTGAAA |
| 3432 | CGCAACCGACATTATATTTCGGCC |
| 3433 | TTGAGGGCGACTGCAACACACAGG |
| 3434 | GCTCGAGTAACACGGTTGACCCGA |
| 3435 | CAGCCCTAGCGCCACGGTAAAATC |
| 3436 | GTCATTAGCGACTTACCCGCCGTA |
| 3437 | CCCAGTGGCCGGCCCTAGATAATA |
| 3438 | CATTCCGTATGCTACTCGCGAACA |
| 3439 | AAGTTTTAACGCTCAAGGGGGCCT |
| 3440 | TTGGCGGTTTCGGTACAGGATCCT |
| 3441 | TACTGCGATGATGGGGATTTGACA |
| 3442 | CGGTGAGCGAAGATCATCCCCTTA |
| 3443 | ATGCAAGTCACCGACCGGCACCTC |
| 3444 | CAAGTGCCGCAATTGGCCTTTTAT |
| 3445 | CCCGTGGTGGATACCTGGGTAAGC |
| 3446 | CCGTCAGGGTCTAAGGACCAGGGT |
| 3447 | CTTTCCGTAGGCGGTGATTTCCAA |
| 3448 | GCTGAAACTGAGATGGTATCCGGC |
| 3449 | CCAACGAGACAGCATGAAGCTCCT |
| 3450 | ATAAGTTCGTGGGCCGGCAAGGTC |
| 3451 | GTGGCCAGGCCATAACTGGTCACT |
| 3452 | CGCTTAGCGCGAGACTCTGAGGGC |
| 3453 | AAGAGCGGCGCCCTAGAACCCAAC |
| 3454 | CCACGGGAACGTCTACGAAATGAT |
| 3455 | AGTCGTGTATCAGGTGCCGAGAGG |
| 3456 | TGAAGCGGCTGGCGATAAGTAGAT |
| 3457 | CTGAGGACGTGCGGTTCATGCTGA |
| 3458 | GAAGGCGTTCGGAAAGTTTTTCGT |
| 3459 | AAGAAAACCACGGCTGAGACCTGA |
| 3460 | TCAGCCGCTGTTGCAGGGAGAAAA |
| 3461 | TTCTGGAAATGGATCGGATAGGCA |
| 3462 | GGGAAATGGTCTTGTTGGCGACCA |
| 3463 | GGTGTCGAAGCCACGATGTATCCC |
| 3464 | CCCCGACTCCCTTCGGGCATAAGT |
| 3465 | CCAAATGCGATAACGCAGCGTGAT |
| 3466 | GCTCGCCAACGTACGAGGCTCAGA |
| 3467 | GGCTTATCAGTCGCCACCAGAGAC |
| 3468 | GATGTGACCCATCCATTCCTGGGA |
| 3469 | TCCTGGTTTGGTATCCCCGAATCA |
| 3470 | CGCCCCGTATATAGCCGGTAAGAG |
| 3471 | GGTTCACTGTAACGATCGCGGCAC |
| 3472 | CCGGTATAGAGGAAACCCGGACGT |
| 3473 | CCTCCCAGGAGATCCTACGCAATT |
| 3474 | TGAAACTCGTCACGCTCCTTGCAG |
| 3475 | TGTTGCGTAACCACCAACCCTCCT |
| 3476 | GCAGCGCAACCTTGTACTTCTTGC |
| 3477 | CGCAAGTGGGAGCCCAAGAGTTTG |
| 3478 | TGCAGGGTAACGAGGGTAAGTGGG |
| 3479 | GAACTGTAGGGTCTCGCCGGTCAA |
| 3480 | CGAGATGTCCAGCAGCGGTTGTTA |
| 3481 | TTGTGGTTGCTCCGGGTAAAAGGA |
| 3482 | TCTACGCATCCCTGGGTAATTTGC |
| 3483 | AGAAGCTGCGAGTCACCGTGACTC |
| 3484 | GGGCGGTGTTGAAGGGCTCTATAC |
| 3485 | TTCCACAACGGGTGAGTAGGACGG |
| 3486 | GCAGCCAGACTGGCCTACCGATCG |
| 3487 | CCCGCCGAGTTGGTTGGCTAAACA |
| 3488 | GCTAGGGTGGTCCTTTCAGTGGGT |
| 3489 | CGTGACTCTCCTTCTTTTCGGCAG |
| 3490 | ACTGCCCATGGGCCACTAGGCTTG |
| 3491 | GGCGTACGAAAAGGCCAATCACTT |
| 3492 | ACTTGTGGTCGACAACGATGTGGC |
| 3493 | CCACCACCCCTGACCCGAAAAAAT |
| 3494 | TGTTGTGCATCACAACATCAGGCC |
| 3495 | GACCACCCGGTAAAGAGGGATGGT |
| 3496 | GCCACCCCTGAAGCACTCGTTATG |
| 3497 | GCTACCAGTTGGAAGACGGGTTGC |
| 3498 | CAACGTTCGCATCCCACAGTTGTA |
| 3499 | TATCGGGTCGTAATGGGCAAAGAG |
| 3500 | TCGGTGTGATTGATGGATAACGCC |
| 3501 | AGAGGTCGAGAGCCCGATAACCTG |
| 3502 | GTAGTTAGGCGCGGCCCTGGCTCA |
| 3503 | TGATTCTCGATGTCACGCCGAACA |
| 3504 | GATGGTTCGCCCTTGTGTCGCAGC |
| 3505 | GCGCAGTTACGTCCATTGTCCCAC |
| 3506 | CCGCCTGATTTAACAAGCCAAGGT |
| 3507 | GACCAAGTGCAGGCGTCAGTCTGG |
| 3508 | CAAAAAAGCAATTCGCCCTGGACG |
| 3509 | ACTGACCTTCTCGCTCTCTCCGTG |
| 3510 | CTCGCCGTGTATCGCTAACCCTCT |
| 3511 | CGGCATTTTTCACATGCTGTGTTG |
| 3512 | ACGTAACGCCTGATGGGGTACACC |
| 3513 | CCCTGTGACCGTGGGAGACACACA |
| 3514 | GCGCATACTCTGGGTAGTCGGCAC |
| 3515 | TCCCCTGCCCATCTCTGAGTTAGG |
| 3516 | TGCAGCGCTAACATAGCGGGTGCA |
| 3517 | GCAGCGTCCACAGGAAACCGCAGC |
| 3518 | AGCGTACCATCGATGGGGATTCGA |
| 3519 | TGGCCTCGCGATCACCACGATGTT |
| 3520 | TTGGTAATCACTCGGCCAGCGCTA |
| 3521 | CGTTAGTAACGATCGTCGGTGCAA |
| 3522 | AATCGCAGATGGTTCGTGGCACAA |
| 3523 | TAAAGCGTCTAGAGGCCGGCTGTG |
| 3524 | TGGCTAAACGAAACTGGGAATCGG |
| 3525 | CCTATGCAGCCACTGGTGTCCTTC |
| 3526 | ACGTGAGATCCAAGGGTGGCTCCT |
| 3527 | TAAACGCCAAAAACCACGAGCAGG |
| 3528 | CCATGGAATGGAAAGCATTGGACG |
| 3529 | ATGATCCCTGGGCTTAGTCGCCTT |
| 3530 | ACCGTATGCCTCAACAGAGTGGCT |
| 3531 | CCACCAAATCGCATAAGCTCCACC |
| 3532 | TCTCAGTTTAATCCCGTGATCGGG |
| 3533 | AAAGGACTACGCCCATCGCTCACA |
| 3534 | CGGGAAGAAAGGCCTAAAGCTTTG |
| 3535 | TTTTGGACATTTTTCTGCATCGGG |
| 3536 | GCAGGGGTCCTTTTCCACGGTAAT |
| 3537 | TCAAATAGGGCGTAGGCAAGCTTG |
| 3538 | ATGAAGTTCCATCCTGTCCGGGCC |
| 3539 | AGAATGATTAAGCGCAAACGCAGC |
| 3540 | GGCAGCAGAGAGTGGCCTAGTTCC |
| 3541 | GTGCAGAGCCGGCCTTATGTAAGA |
| 3542 | CATACGGGTATGGCGATGGTTACC |
| 3543 | AAGAACAGGAACCGCTGACAAGGA |
| 3544 | GATGTGTGTCGCGTCCTTAAGGGC |
| 3545 | TATCCATGTAAGGCTCCTGAGGCG |
| 3546 | AGTTTTTTCCTAAACGATCCGCGC |
| 3547 | CTGACCGGACGACCCAGAATGTAT |
| 3548 | GCATGTGGTCAAAGCTTGTCGATG |
| 3549 | CAGAAGTGCATGGGTTCGGATGAA |
| 3550 | ATAGCGTACCGGAGGGCTTACCAG |
| 3551 | AAGACTTGGCGCTTGTGGGTAAGG |
| 3552 | TATTGTGGCGCCTCACGCGCAATC |
| 3553 | TCGGCCATGGGATTTCACAAAGTC |
| 3554 | TGGTCGGTGCCGTTTCACCTTTAC |
| 3555 | CATTTCCGCGGGCAGGAGAAAGAT |
| 3556 | CCTGAGTCGCGATACGACTCAACA |
| 3557 | AGGTGTACCGCCGTCGGGTTATAC |
| 3558 | TCCTTGTACGAGCCAAGCCTGGGT |
| 3559 | AGAAGCCCGAAGTCCCGTGTAGAC |
| 3560 | AGAGGGGCCCTTAGGCAAATACGT |
| 3561 | ATGCGGCAACATCCGATCGTAGAT |
| 3562 | CGCAGTGGGCAGTAAAGACAGAGG |
| 3563 | TCGGGTAGTGCAAACCTCAATCGT |
| 3564 | TCTTCACTGTGGTGGACTTGGGG |
| 3565 | GTCCCAGGGCGATTGGTACTAAGG |
| 3566 | GGTAGATCCAGCCATTGGGACCTC |
| 3567 | GGGGATTGTGCGCTCCAAGGACCC |
| 3568 | CTCTGTCCTAGACTGAGCCGTCGC |
| 3569 | CGATGAACAAATGAGTGCGTGTGA |
| 3570 | GAGGTCGAGCTGCCTGAGAGGAGT |
| 3571 | CAGTGGGACTGCTAACGTGGGTCA |
| 3572 | GAGTCGCTCGAGGAACTACGGCCG |
| 3573 | CGGCTACGGAATGATGCAGGATGG |
| 3574 | TCGCTCTCGCTATGGCAATTCTGG |
| 3575 | TGAATCACGGCCCTCTCTGGTACA |
| 3576 | CAGGTGCCATCGAGCGCTTTAGTG |
| 3577 | TGGGAAAATCGAAATCGTCAGGAA |
| 3578 | CGGGGAGGAAGATGTTCCAGCGGT |
| 3579 | TGTGGACCGGTGGTCACGTCTTTT |
| 3580 | GCACGTCTCGCAATCTGCGATCAG |
| 3581 | CCTAATGCCGTATCAGCGACCAGA |
| 3582 | ATAACGCGGGTGAAGGATTCGTCT |
| 3583 | TTCAACCTTGTGGGGCGTCCCACT |
| 3584 | CTACTTCCAAATCTCCGCGTCGGT |
| 3585 | AGCGAACGCACTGCCAGTGGATAC |
| 3586 | GAAAGTGGCGGCGAGGAAAAACAC |
| 3587 | CAGGGGGCGCATATTTGACAGATT |
| 3588 | TAACTCGCTGCCCTCAACTCAGGG |
| 3589 | TCGATTGTTGGGTCTACCGTGGTT |
| 3590 | GCTGGGATTAGTGCCGGGTAACCG |
| 3591 | TGGTTGCAACATCGCGCTATTACG |
| 3592 | GGGCGTGCTTTGAGCTGAAGCGTG |
| 3593 | ATGTTGAGGTTAGTCCCCGACCGT |
| 3594 | GACCGCGTAGTTAGCAATGTTGCG |
| 3595 | CCAACCCACTGACATCGATGGAAA |
| 3596 | TGCTGCTATTGTCGCACCGATATG |
| 3597 | TACAAAGAATCGGGACCTGCGACT |
| 3598 | GCGCCTCATCCCGCATCGAATTAT |
| 3599 | CGAGGGATTTTGACCAGTGGATGA |
| 3600 | TGATAGGCATACGCGGAGAAGTCC |
| 3601 | CGAGTTGTCAACGGCCATCGAATT |
| 3602 | CCCGCACCGGATTATTAACGAACC |
| 3603 | TCGTCCTTGGGTCCCATGTAGAAA |
| 3604 | TCACGAAGCATCTTTGCGACGTAA |
| 3605 | TGTAAGTTGCCAACTTTGCGGGTT |
| 3606 | GCACACCACCGGCAGATATCAAGA |
| 3607 | GTGTGGTTTGTGPATGCGTGGTGA |
| 3608 | CAGCTGCGGCCCCACCTTCGATAC |
| 3609 | CAGCGAAGGACGACTACTGTGCAC |
| 3610 | CAGCAGTTCGTTGCTTCCTGATTG |
| 3611 | AAACAATGGAGTGTACCTCCCGCA |
| 3612 | ACTATACGAGCATCATGAGCCGGC |
| 3613 | CTTGATAAGGTGGGATTCCGGGCA |
| 3614 | TTTAGTAGAACGCTGCGCGCGGTG |
| 3615 | AACTGACGTTGAATAAAACCGGCG |
| 3616 | GCTTTGTTCTACCGCGGATCATCA |
| 3617 | TGATATGCAGCGGCTCGGCCTTAT |
| 3618 | CGGGAGTGCGTTTATGTCCATGAT |
| 3619 | CAAATACCGGGAACGGATCGAAGC |
| 3620 | GATCAAGCCGAATGCTTTGCAAAG |
| 3621 | AGAGAGGATGCGCTCCGGTTAGAG |
| 3622 | CTTAGTCAGCATACCCGCGGGCAG |
| 3623 | GTGTCTCGGGGCGCAGGACCTGTA |
| 3624 | AACGCTCCACTGCCGTGATTCACT |
| 3625 | GATCGTTGAGTCATCCCGTGGAGT |
| 3626 | CCTGGCCGGGTGCAATACTACAGT |
| 3627 | CGTAGCCCGAACGTAAGGGTCAGC |
| 3628 | CTGTGGCTTCAAGAGGATCCGTTG |
| 3629 | CTTGGGTCGGTGTAATGTCCTCGA |
| 3630 | GCCGTTGTGCGCTATTCTTACGGA |
| 3631 | TCGCACGATGGCTAGAACGAGTAA |
| 3632 | ATTTGTTGCAATGGGATGGCTCTG |
| 3633 | CGAATATCCGCTCGAACCTGACAA |
| 3634 | AAGTGGCGTGCGTCATAGCGCGAC |
| 3635 | TGATGTCCCTCCACACCGTGAACT |
| 3636 | CAAATGAAGTCGGGGCCAATATTG |
| 3637 | GATGCATAGCGTGATTCCGGTGTA |
| 3638 | GTGACCGTAGAAGCTCACCAGGGC |
| 3639 | ATAAGGACATATTCGGCCTGGGGA |
| 3640 | AGATCTCACAACCGGAACCGGACG |
| 3641 | GTTGCGTTTGGGGGCGTCATACAA |
| 3642 | TGTGAGGTTTTCCTAAGGCGAACG |
| 3643 | CATCTTGGTTTGCGAACGAACTCA |
| 3644 | TTCCTGTCACAGATTCGTGGCCTT |
| 3645 | AACTTACCGATCCCTGAACGTGCA |
| 3646 | CCTATTCTGGACATGCGGCCACAT |
| 3647 | GTCGATGGGGAGCTCCAGTTGCAT |
| 3648 | CGACCGTGAGGGTCCATACGTAGA |
| 3649 | TCTCGTTTGCACGCAACTGGGCCA |
| 3650 | ACTCCGCCGAATGAAGGAATAGCT |
| 3651 | CCTCGACCTGGCGTGATGGAAGGC |
| 3652 | TAACAGCCGTTTTGCGGTTCACAA |
| 3653 | GCCTCCTGCAGTACGGTGTCTGTT |
| 3654 | GGCAGTCGGTCCCACTTAGTTCGA |
| 3655 | TAATCCACGGCTTTGGTGGAAGTC |
| 3656 | CGGTGCAAGATCCTGGTTGTGTGA |
| 3657 | TTTCACCACTACCTTAGGTCGGCG |
| 3658 | CATCCCGTACCGGGAGGACAAGTC |
| 3659 | ACGAGGTAAAGGGATCCGTGCTGG |
| 3660 | CTAATAGTTTGGCAGAGGGGCGCT |
| 3661 | AGCATGGTAACCCTGAGCCAGCAG |
| 3662 | GGAATCCTTGTGGGAACAGCCGAT |
| 3663 | CTGATGTGGGAAAGAGGGTGGGAC |
| 3664 | ACTTTTTGCAATCCCGGCGTTGTA |
| 3665 | GCGATGACGTGACGAGTTCTCACC |
| 3666 | CCAGGTATTGAGCCCCGCCATATA |
| 3667 | TTGGACGTCCTCCGAATATTGGCA |
| 3668 | GGTAAGTGCGGGAAGTACGCTGAC |
| 3669 | CCGCCTGAACCGTCGTAGGGATTA |
| 3670 | CGTTTTTGAGTAAGGATTGGGCGA |
| 3671 | TGTGGTATTGAGGCATAGGTGGCA |
| 3672 | TCCGGAAGGAAGGCGCGATATGGC |
| 3673 | GTTGAGCGAATCGGACGGCTTTAC |
| 3674 | TGAGTCTCCGAACGACAAGCGATC |
| 3675 | AGTGAAGAGGGAGAGTCCAACCCG |
| 3676 | GTGAAGCCTGACGAATCCAACGTG |
| 3677 | GTGCAGGCCTGTATCCCCATGACT |
| 3678 | GTGGGTTTCCTACACACCGGATGA |
| 3679 | GCGCCGTCGACTCTCTTCAGCTGC |
| 3680 | CTAGGCCTGCCATCACTGAGCAAT |
| 3681 | TTGGTGATGACTCATGGCCAGACC |
| 3682 | TATCTCCCGCGGGGTATATTACCG |
| 3683 | CCGAGGGACACGTATCCCTGTTCG |
| 3684 | TATCCCGCAGCACGCATTCGATCT |
| 3685 | TGATGATAGAGCAGGGTGCCGTCA |
| 3686 | GTAGGAGCACACATTCGGATTCGG |
| 3687 | CCCTTACTACGCCCAGCCCTTTTG |
| 3688 | GTACCAGGGGGTGTGCTCCAAGGG |
| 3689 | TGACCAGGCGGACCAGACGGTTTT |
| 3690 | CGTAAGCGGCGGTAGGTGTGCTAC |
| 3691 | CGCGGGGAGGGATCAGCAGTTTTG |
| 3692 | AAAGCGTATCCAGAAAGGCCATGG |
| 3693 | AAGAAGAGACGCATGCTTGGACGT |
| 3694 | TGGCCATTTGCGGGAGGTGGCTTA |
| 3695 | AACGCCGAATTGAGGAGGCGGTTA |
| 3696 | GCCTCATTACGACATTGGCAGCAT |
| 3697 | TCGAACGCGATTTTGGAAATGCCC |
| 3698 | AGGAATTCTAGCCGAAAGCCCTGC |
| 3699 | TCCGCTGGTTGGGTGCTCTGGTTG |
| 3700 | GTCGCGCTCCGTCCGATAGTATGA |
| 3701 | TGTGCAAGGACGGATGATTGCACT |
| 3702 | GGACAAGCGGCAACCTGGGAGAAG |
| 3703 | ATGCGGTGGCTACGGACTAATCCA |
| 3704 | TGCACGCAGGTGGAAAGCAGGCTT |
| 3705 | AGATTGTGGGAGTTGTCACGCTCC |
| 3706 | AACAGCAGTGAGGGCTGAAGCTTG |
| 3707 | CTGCCTGTTTCCTTCACGCTCCAT |
| 3708 | CCAATCCACTTGAGTCAACTTGCG |
| 3709 | CATTCTACCGCCCAACTTTTGCAA |
| 3710 | CGGAGAACCATGCTGAGCAGTCCA |
| 3711 | GACTGTTCCTCCAGAAAGGCGCAT |
| 3712 | AAATAATTGCTCCACGCGAAGCGC |
| 3713 | GGGCCTGGAAGACCAACCAAATAC |
| 3714 | ACGACGCGAGCACGTAGATATCAA |
| 3715 | TACGGGATCCTCGTGGCTACATCT |
| 3716 | CAAAGTCTCCCCGACCGAGTTGAC |
| 3717 | CCCGAGGCGAAGATCTCTAGGCAC |
| 3718 | CAAAATTCTCGCCACGAGACCCTA |
| 3719 | CTGTGCGCATTCCAAACACATCAC |
| 3720 | CATGGAAATGCCAGCTGCCTCCAT |
| 3721 | CGCGAAACCACAGTCCTCGTCGGG |
| 3722 | GTCCGCAGCTGTCCCGACATTGGT |
| 3723 | GTCTCATTGGGACGATCGTCTCGA |
| 3724 | AGAGCGTTGCATGCTTGGCTGCGG |
| 3725 | CTTCCGCCCCTGTTCGCAATGAGG |
| 3726 | TTGCGGTTCATACCGAAGCCAACA |
| 3727 | TGCGCGAGAATCGTTCGTACGACG |
| 3728 | TGTATACCGTAGGCGTCCGTGGGG |
| 3729 | TGCGGGGTATAGGGCTTCCTTATG |
| 3730 | ATCCCAGCCCAAGCAGCAGACGCA |
| 3731 | GTTCTTGGCCACAGGAATGGCCGT |
| 3732 | CACATGGGCATTAATTGCTACGGC |
| 3733 | ATAAGTCGGTCTGCCTGGCAATGA |
| 3734 | ACCTCGAGGCTGAGAACGTCAAAA |
| 3735 | GCGGAACGCTAGCCCCTTATGGTT |
| 3736 | TGCGAGGCTCCTGGAGCAATCCAA |
| 3737 | ACAGAAGGGCGATCGCTCTGGCTG |
| 3738 | GGTTGGCAAGGGGCCAGCTCCTAC |
| 3739 | ATCGCTTCGCTCTATGGAGTCCGA |
| 3740 | CGTCCCGATAGGCCGCCTTGATCT |
| 3741 | GAATTCTGAGGCGGCATTGTCCAC |
| 3742 | CAGCCCATCAGTATCGGCTGCGTA |
| 3743 | TGGAGAGTCGGATCCGTAGCGTCA |
| 3744 | TGGATCCAGTGCGAGTCTTGGCCG |
| 3745 | ATGCGGTCGTGCTTGGAATCCTCT |
| 3746 | ATCGCACTGCCGCGTCATAACAGC |
| 3747 | CACGTCTCCGCCGGAACACAACTG |
| 3748 | AAGACAGTGGGTGAACGCACGGTA |
| 3749 | ACGCGCATAGGTGGTCAAACATCG |
| 3750 | CCCGGCGGTAGAAATTGACAACCT |
| 3751 | AAGGGATACTCAGGCGCCTGTTTT |
| 3752 | CTTCTCTCTTGTGCGGGCTCCCGT |
| 3753 | TTGAAGGGACCTGCCAAATGGCGA |
| 3754 | ACGCATGACGACGTCCAGTACGGG |
| 3755 | AAATGGATGTTACGCCGGCAAGCT |
| 3756 | TCGTGCGAGGCCTCTTCGGCATAC |
| 3757 | TACATCGCGTCGAGTCATTCTTGG |
| 3758 | TCACACCACATAATGGCACCACGT |
| 3759 | CAGGTTCACGGTTGAGGAGTGCGA |
| 3760 | GGTGTTACACCGCTTCGTTGTCCT |
| 3761 | ACAATAATAAGGGAGCATCGGCCG |
| 3762 | TCGGGTCCTATGATCCAGTCCCAA |
| 3763 | ACCCATTCCTCCTGCGGCGATCAA |
| 3764 | TCGCAGGTGTAGACGGACGAAAAG |
| 3765 | CTCTTGCGTAGTAATCGGCCCGCA |
| 3766 | TTCCGTGTCACGCGAGCCTGCTTT |
| 3767 | ACTCTAAGTAGGGCTGGGTCGCGA |
| 3768 | TTGGTGGCTGTAAAGGTGCTTGGC |
| 3769 | CCGAATTACCCATTCATACGGCAC |
| 3770 | GATGGATAGGTTCGCTTCCCGCAA |
| 3771 | ATGACGGAAAGAATGTGATTCGGC |
| 3772 | ACGGTTCGGCTTCTGTTAGTCACG |
| 3773 | GGATCCCGTAATTGAGGCGGCCAC |
| 3774 | ACCCGTTAAGTCGACGCCTGCGGG |
| 3775 | TTCGATGTGAACGGTTGGCCAACC |
| 3776 | TCGATCGGGAGTCTACCGCCATGT |
| 3777 | AGCAACGAGTTTATGAGCGCAGGA |
| 3778 | TGGGAAACGAATGGGTGGCGGTTG |
| 3779 | TCTGTGTTGCCCCACCTACAGCAA |
| 3780 | CCTGCATTGGATGTACCCGCGGGT |
| 3781 | GAACGAGGTCCGGGTTTGCATCTC |
| 3782 | GGCGCCGAAGCAGAACGACCATAT |
| 3783 | AGGCATCACGCATCAGGTACTTGG |
| 3784 | TTTACAAAAGCATCGGCCCTGGGA |
| 3785 | CCCAGGCGGTCAACCAATTGTAGA |
| 3786 | CTGCAGCACGTGCCTGAAATTCGT |
| 3787 | CCGTTTTGCTCCAGCTATGAGCGT |
| 3788 | ATTTGTGCCGCATTGGGGTTATTC |
| 3789 | TAAGCAGAAAGCCGCAACTCCGGT |
| 3790 | GCGACTGATATAGTGCTCGGACCG |
| 3791 | AACTCTATTCTGACACCGCCCGAA |
| 3792 | GTGCGCTCCAAGAAGAAACACACC |
| 3793 | ACGACCAGCGGTCTGAGATCTAGG |
| 3794 | ATCCCCTCCTCAGGTCGACGCTGT |
| 3795 | TGACATACGCGTCACCCAGCACAG |
| 3796 | TAACCGCGACTCTGACTCCCTTGT |
| 3797 | AAGCGGTTTGATCTGTGCAATCGG |
| 3798 | CTGTCAACTCGGTCGTCCGCACAG |
| 3799 | AACTTTGCCGTTTAGGGCAGGTGA |
| 3800 | GCTGAAGAACTCCCAATTCGCTGG |
| 3801 | AAGATGCGATGGGTCAGTCCTCGT |
| 3802 | ACCCACCTCTGAAGGTTGAGACGG |
| 3803 | AGGCTACGCACCCTCGAGAGTGAC |
| 3804 | CGGTCACGAACGTGGTCCAGTTTT |
| 3805 | CAAAGCAACGCGCGCCACTTAAAA |
| 3806 | ACGAGGAAGGAACTGATCCCCAGT |
| 3807 | TTCGCCACTATGGGCTCAGCATTA |
| 3808 | CGCTCGGCAGAGGAG~CCACTCAC |
| 3809 | TGTTGGCACGACTCCGTCCATGAA |
| 3810 | TGCCTACCCGGTGATTGCGACATC |
| 3811 | CAACGGTCGGATCTGAGGAGATCT |
| 3812 | CGTTACGAAGCGAAGTTCCCGAGT |
| 3813 | AGTGACGGCCAAAGTCGCCATTCT |
| 3814 | ATTCAGCTGGGCATAGGCGATGGG |
| 3815 | TAGGACAGCGTGGCTGGCTACACA |
| 3816 | AATTTGTCCAGCTCTGCACGACCG |
| 3817 | TGAGTGGGCTGTGATCCGTTCCAC |
| 3818 | TGTGGTGACACGCCAGAGCTGGTT |
| 3819 | CCTCACAGGTGTGAGAGGAGCCGC |
| 3820 | AGTCCCGCTTCTGCAAATTCCGAA |
| 3821 | TCTGCGCCTACCCGTAAGCTGAAC |
| 3822 | GCCTCCTGAGTTGATTCATGCATG |
| 3823 | CCTAACGGTTGGTTCGCCGTTTTT |
| 3824 | TCGCAAACCCACGAATGAGTCCCG |
| 3825 | AGTGCTAAGGTGGGCGAGCAGAGG |
| 3826 | CTGGAGACTGCGATGGCAGGGTTG |
| 3827 | AAGGGATAGTGATGGCGATGGACG |
| 3828 | CTATCCACGGTGATGTCCGCCATT |
| 3829 | CGGACTAGAACTTGCCAAGCACGA |
| 3830 | AGAGCCGGATGGCATTGCATGAAC |
| 3831 | AGTTGGCTAGCGGTCGAATGAGCA |
| 3832 | GCATGCGGTCACCGCTTCATCTAA |
| 3833 | GTGAGATTCCAAGCTCGCCGGTGA |
| 3834 | GCCATCCACCGCACAATGAACGCT |
| 3835 | GGGTGGTCCTCACTGTGGTTGGCA |
| 3836 | AGGCGGCTACGACGAGCGTCGTTA |
| 3837 | GCCAAGTGATCGTGCTTCCGCGTA |
| 3838 | TAGCCGTTTATTCCCTTGATGCGC |
| 3839 | ACTATGTGGGACGAGCGTCTGCGA |
| 3840 | GCACCTTCGAGAACCCATCAGATG |
| 3841 | ATTTTCTGTACCGATGCTCACCGG |
| 3842 | CACTGGAGCAATAAATGGCCAGGC |
| 3843 | GGGTTCACGTATCTCATGGATGCG |
| 3844 | GCACGCTCCCAGTATGCTCCTTCA |
| 3845 | GAAGGGACTTAGTCCGCGGCCCTC |
| 3846 | TTCGTTACCCTAAGGGCGTTTGCA |
| 3847 | GTTCCAGGTCACGACGAGCTGCGC |
| 3848 | TCGTACGTAGTCACACCGCGACTT |
| 3849 | GGGCTGGAGTAGCGGTCTGCTATG |
| 3850 | TAGCGGCACTCGTGTTGCGAGTGG |
| 3851 | ACGTTGGGTTCTGACACGGCGATT |
| 3852 | TGTTGCTGCGCCCCAAGTGATCTT |
| 3853 | CCCAGGTCGTTACGGTGCATCACA |
| 3854 | CCTAGTGCACAGGCAAATCGGGCT |
| 3855 | GGCGTTCTCCAAGATAAGGCCAAA |
| 3856 | ACTTCGATACCGTGGACCTCGCCA |
| 3857 | CTGAGCGCGCTAAACGTCCCTAGC |
| 3858 | ATCAGATAAACGATCCGACGCGTC |
| 3859 | CATGGCTGAATTTGTCGACCCTCT |
| 3860 | CGAAAGCGAGCAAATAGAATCCCC |
| 3861 | AGATTGCCCTGCGGCAGGTTGAAT |
| 3862 | AAGAGGCGGCCGATCAGTTAGAAA |
| 3863 | CTGATGCCTGTAAGGAGGCGCTCG |
| 3864 | AATCGCGAGGTTCGGCAGACAAAG |
| 3865 | CGTTGGGACACGGACCGTTCACTC |
| 3866 | AGATGTGTGCACTCGCGGTCATTT |
| 3867 | CAACTCGAGTGGCGGTAACATCTG |
| 3868 | ACCAAGGTTGCGATTACGGGAAGC |
| 3869 | CGAAGCGGTAGACGGCTCGCGTTA |
| 3870 | TCTCGCGAACAGGAGGGAAGGCGT |
| 3871 | GTCCCGATTTGCGCTGTGAGGAAA |
| 3872 | TACCACGCGTCGGCACGGAAATGG |
| 3873 | AAATGCTACCCGATTGCGCGGGAT |
| 3874 | TCGATTCAGGTTTGTGCTGCGGAG |
| 3875 | CCATCTCATCCCACTATGGCATGC |
| 3876 | CTGGCCCGTGTTTGGTTGAGTCGA |
| 3877 | GACACACACGTTGCAGGGCTTCCC |
| 3878 | TCGAATCGAGTCGATCGTGAAGGT |
| 3879 | GAAAGCACTCGATCGCGTTGGATT |
| 3880 | AATTACGCGAACATGGGGCGTCAA |
| 3881 | GTGCTAACACTGTGGTCGTTCCCA |
| 3882 | GGTAAGCGCCAGCCAGGAGTTGTC |
| 3883 | GGCGATCGTTCAGGAATCGCGTCA |
| 3884 | CTGGCTAGACCTCCGACACAGGCT |
| 3885 | CGGGTTAAACGCCAACTGGCCTAG |
| 3886 | ATCGCAGCCTGGCCGCCTAGTTTT |
| 3887 | GGCGTAGCCTAGCAAATTATGCCA |
| 3888 | ATGACGCGACGGAGACAATACGGC |
| 3889 | GTTGCATCACGAAAATGCCGTCTT |
| 3890 | GAGTCATGCGTTCCTCGCTTTACC |
| 3891 | TCTGAACCGGTTATCCCCAACCTC |
| 3892 | TGCCTCTGGTAGGCGCCCAGTTAC |
| 3893 | CTGACGGTTTTCATTCGGCGTGCC |
| 3894 | TGAACACGAGCAACACTCCAACGC |
| 3895 | CGGCGCGCGAAAGACTTGAACTTG |
| 3896 | GCTACGAGTACCCGTCGGAAACGC |
| 3897 | ATACCCAACAGCATGGAGCGACCA |
| 3898 | ATCGCATCGCATCGTATTCACGGG |
| 3899 | CGGCCTAGAGGTGCGAAAGCTATC |
| 3900 | TAACGCTTTTCCGAGGCCGA1TCT |
| 3901 | TCTGTCCTAGCACGCCGACCTGCT |
| 3902 | CTCATCGTTCAGTCGGTCGTCGTA |
| 3903 | TCGTCGAGCAGATAGCGGGGTAGG |
| 3904 | TCGACCACAGTCAGGACACTACCG |
| 3905 | TGCGATTCTATGATGTCCGAACGC |
| 3906 | CAAATGCAATGGCAAGCACTCACC |
| 3907 | TCTAATCCATCGTTTTTTGGGCGA |
| 3908 | TCTCAACTCCGGTACGACGAAACA |
| 3909 | CTGAAGAGGGTAGCCTGGGAGCGG |
| 3910 | GGCACAATTAAAACGCGCCGCGTT |
| 3911 | CAAAGGAGGGTCAAAGGCCAGAAA |
| 3912 | TTTGCGGCCGTGACGAGCAAAAAT |
| 3913 | AGGAATGTGCGTGGCACCTGTGGA |
| 3914 | TCGTGATGACTGCCTTCCGAATCA |
| 3915 | CACGTCGACATGTTTGGTACCTCG |
| 3916 | TTGCGGTAGTTTGGTTACCACCGT |
| 3917 | GCAGTGGCGACAAATACAGCTGAG |
| 3918 | ACGGCATGATGGAGGGATAAACGT |
| 3919 | TGGGATAATCCGCAAGCGCATAGC |
| 3920 | CCTAGCTCTGCTGCGTCTTTGCGC |
| 3921 | TCCTGGAACTGCTGAAGGCGACTT |
| 3922 | CGAAGGCGGCATGGTGTAGTCTCC |
| 3923 | AACATTGTTCCCATCCCAGAGCAC |
| 3924 | CCAGGCAAGAAACAACCACGCGCT |
| 3925 | AAATCCACAGGCGCGCCAAAGCTG |
| 3926 | GCTCACCGCAGACTCCGCGCGATA |
| 3927 | TAGGTGGCGAGAGAGCGCCCACAA |
| 3928 | GGCGTTGGTGTGTCGGGACCATGA |
| 3929 | TCTGAATGCTTCCGTGCTTTCGTG |
| 3930 | ACGCTCTGGACCTCGCTCATTCGA |
| 3931 | TCCTTTATGCGCAGCGCTCGTGTT |
| 3932 | TTGCCGTCCTGCAGCAGGTAGCTC |
| 3933 | GGTCTAGTGGCAGCAAGGAGCGAT |
| 3934 | GGTAACGCGACCAGCTTAGACACC |
| 3935 | GTGGCGATTGGCTTCCTATGCATA |
| 3936 | TCAAAATACGGCCAGGAAGGGCAA |
| 3937 | TGCCATGCAGTCAGGTACGATGGT |
| 3938 | ACAGGTTACGTCGTGTGTTCCCGT |
| 3939 | CTCATGACGAACGAGCGGTCTGCA |
| 3940 | GTCGTGCGAGAGGCCAAGACCTTA |
| 3941 | GCTGGCTGACGCTGTTGTCAGAGG |
| 3942 | GCTACAGTGCTGCGTCCCGTGCCT |
| 3943 | TTTACGAGCACCAAGCTGGCGTAG |
| 3944 | ACGAGTTGACGGTCGTAGGGACCG |
| 3945 | TCGGATGGTAGGAGGCGAGATCGG |
| 3946 | ATTATGCAGATCCTGTGCATCCGC |
| 3947 | AGGGATGGAGACGAAGGAAGCATT |
| 3948 | ACCCCAGGACCCGTATTCCCTAGC |
| 3949 | GCACCATCCTGGGGCTTCTCAATG |
| 3950 | TACAATCCGTGGACGTTTGCTCAG |
| 3951 | GGTAGGCGAATCCGACTGGCATAG |
| 3952 | AGGACCGAACCCATGTGCAGCATC |
| 3953 | ATACACCGCACAGAAGCACAGCTG |
| 3954 | TCCTTGGCGGCCGTGTGTTTATTG |
| 3955 | CTCCACGCGAAGGGCGCTTGTAAC |
| 3956 | TGGCCCTGCCATCCTCGGATTCAG |
| 3957 | TGTCTATTCGCCAGCGTGAGCATC |
| 3958 | TGTTGTTGGCACGCCTCTACGGCA |
| 3959 | GTGCCTCAACCGTATCGTGGCGGT |
| 3960 | TCCTCGAAGTAGCGTGACCGPACC |
| 3961 | AAACAATTTCCTGCACTCTCGGCC |
| 3962 | CACAAACTCGTCGAGGCACACAGT |
| 3963 | GACGAAACGCTCGGCAGAAAGCCT |
| 3964 | TCAACTCACACGGGACAGCAGTTC |
| 3965 | TCACGTGGATGGGCTTAGCTGGGC |
| 3966 | AGGTGTTTGTTCCGACTGGCCACA |
| 3967 | TCAACCCTCTATTCCCGAGCATTG |
| 3968 | ACCTCACACAAGCGTTCTCGTCGA |
| 3969 | AACAGCATGCGGTCGCTGGCTTTC |
| 3970 | CACGGACACGTGTTACATCCGATG |
| 3971 | CTGGGAGCCTGCTGATACATGGTG |
| 3972 | CGTCCTATGGGCCATGGCCAGGAT |
| 3973 | GTCCCCAAATCTCGCTTTACAGGC |
| 3974 | TCACAAACCTGTGCGTGCATTGTC |
| 3975 | CACACTCGTGGCCTGCGTTGGGAA |
| 3976 | GCCTGCACTTACGGCTATCTCGCC |
| 3977 | TTGGCGTGGCGATTACCTGTTATT |
| 3978 | TTTGCGGCTGAAGTTTACAGGGTG |
| 3979 | CACTTAAGGGGCTGACCGAGCAAC |
| 3980 | AGAAAACGTCAATCCGCCACCTTT |
| 3981 | AACAAAACGGCGCTCCAACAAACG |
| 3982 | GCCTCAATATCTGGTTGCCGCCTG |
| 3983 | TTCCACAGTCAATGATGGGCGTGC |
| 3984 | GATTCCCAGTCTACCCGCGAGCAT |
| 3985 | AGGCCAATTACGACCCTGTCACGG |
| 3986 | CATGCGAACGTTCCGAGGAGACGG |
| 3987 | CACACGCGATGGGTTGTGTGACGC |
| 3988 | TCCGGTATTGCGCAGGAACCATAG |
| 3989 | AAGATTAGGTGTGCCCGCCTCAGG |
| 3990 | TCGTTACGCCCCGACTCGACGATG |
| 3991 | ACTAAAATCGCCAGGTTGCTCCCT |
| 3992 | AGGATGGCCACGCCGAATCAAAGT |
| 3993 | TGATGAAGCAGCTCATCGCTGGCG |
| 3994 | CCCCGATGGGTCTTTGTTGGACTC |
| 3995 | ACACGAGGGCTGCTGGTGAGGGCT |
| 3996 | TGGTCACCAATTTGATGATCCGAG |
| 3997 | AAGGCCGCTTGCATGCGACAAATT |
| 3998 | CCAGTGTTCGTTCATCGGTGGCGT |
| 3999 | CCGACCGCTACATAGGTGTGCGAA |
| 4000 | TGTTGAAGCCGTTCCCAGATGACA |

TABLE 2

| Seq. ID No. | Decoder Sequence (5′-3′) | Probe Sequence (5′-3′) |
|---|---|---|
| 1 | TTCGCCGTCGTGTAGGCTTTTCAA | TTGAAAAGCCTACACGACGGCGAA |
| 2 | TTCGAAGCGCACGTCCCTTTTCAA | TTGAAAAGGGACGTGCGCTTCGAA |
| 3 | AACGCGTGGGGAATGGGACATCAA | TTGATGTCCCATTCCCCACGCGTT |
| 4 | CCGTCGCATACCGGCTACGATCAA | TTGATCGTAGCCGGTATGCGACGG |
| 5 | ATGGCCGTGCTGGGGACAAGTCAA | TTGACTTGTCCCCAGCACGGCCAT |
| 6 | TTGCAACGGGCTGGTCAACGTCAA | TTGACGTTGACCAGCCCGTTGCAA |
| 7 | CGCATAGGTTGCCGATTTCGTCAA | TTGACGAAATCGGCAACCTATGCG |
| 8 | CCGTTTGCGGTCGTCCTTGCTCAA | TTGAGCAAGGACGACCGCAAACGG |
| 9 | TTCGCTTTCGTGGCTGCACTTCAA | TTGAAGTGCAGCCACGAAAGCGAA |
| 10 | GTCCAACGCGCAACTCCGATTCAA | TTGAATCGGAGTTGCGCGTTGGAC |
| 11 | TTGCCGCACCGTCCGTCATCTCAA | TTGAGATGACGGACGGTGCGGCAA |
| 12 | CATCGTCCCTTTCGATGGGATCAA | TTGATCCCATCGAAAGGGACGATG |
| 13 | GCACGGGAGCTGACGACGTGTCAA | TTGACACGTCGTCAGCTCCCGTGC |
| 14 | AGACGCACCGCAACAGGCTGTCAA | TTGACAGCCTGTTGCGGTGCGTCT |
| 15 | CGTGTAGGGGTCCCGTGCTGTCAA | TTGACAGCACGGGACCCCTACACG |
| 16 | CATCGCTGCAAGTACCGCACTCAA | TTGAGTGCGGTACTTGCAGCGATG |
| 17 | GGCTGGTTCGGCCCGAAAGCTTAG | CTAAGCTTTCGGGCCGAACCAGCC |
| 18 | GTTCCCAGTGAAGCTGCGATCTGG | CCAGATCGCAGCTTCACTGGGAAC |
| 19 | TACTTGGCATGGAATCCCTTACGC | GCGTAAGGGATTCCATGCCAAGTA |
| 20 | ACTAGCATATTTCAGGGCACCGGC | GCCGGTGCCCTGAAATATGCTAGT |
| 21 | GAACGGTCAATGAACCCGCTGTGA | TCACAGCGGGTTCATTGACCGTTC |
| 22 | GCGGCCTTGGTTCAATATGAATCG | CGATTCATATTGAACCAAGGCCGC |
| 23 | GATCGTTAGAGGGACCTTGCCCGA | TCGGGCAAGGTCCCTCTAACGATC |
| 24 | TGGACCTAGTCCGGCAGTGACGAA | TTCGTCACTGCCGGACTAGGTCCA |
| 25 | ATAAACTACCCAGGACGGGCGGAA | TTCCGCCCGTCCTGGGTAGTTTAT |
| 26 | CATCGGTTCGCGCCAATCCAGATA | TATCTGGATTGGCGCGAACCGATG |
| 27 | GTCGGGCATAGAGCCGACCACCCT | AGGGTGGTCGGCTCTATGCCCGAC |
| 28 | CTTGGGTCATGATTCACCGTGCTA | TAGCACGGTGAATCATGACCCAAG |
| 29 | TGCCTAACGTGCTAATCAGCAGCG | CGCTGCTGATTAGCACGTTAGGCA |
| 30 | CGCATGTTGGAGCATATGCCCTGA | TCAGGGCATATGCTCCAACATGCG |
| 31 | AGCCACTGCATCAGTGCTGTTCAA | TTGAACAGCACTGATGCAGTGGCT |
| 32 | GGTTGTTTTGAGGCGTCCCACACT | AGTGTGGGACGCCTCAAAACAACC |
| 33 | TCGACCAAGAGCAAGGGCGGACCA | TGGTCCGCCCTTGCTCTTGGTCGA |
| 34 | GACATCGCTATTGCGCATGGATCA | TGATCCATGCGCAATAGCGATGTC |
| 35 | GAAATACGAAGTCTGCGGGAGTCG | CGACTCCCGCAGACTTCGTATTTC |
| 36 | TGTCATGAATGATTGATCGCGCGA | TCGCGCGATCAATCATTCATGACA |
| 37 | ATATCGGGATTCGTTCCCGGTGAA | TTCACCGGGAACGAATCCCGATAT |
| 38 | GCGAGCGTACCGAAGGGCCTAGAA | TTCTAGGCCCTTCGGTACGCTCGC |
| 39 | TTACCGGCAGCGGACTTCCGAATT | AATTCGGAAGTCCGCTGCCGGTAA |
| 40 | GTAATCGAGAGCTGCGCGCGGTCT | AGACGGCGCGCAGCTCTCGATTAC |
| 41 | CCTGTTAGCGTAGGCGAGTCGATC | GATCGACTCGCGTACGCTAACAGG |
| 42 | TAGCGGACCGGCAGAATGAGTTCC | GGAACTCATTCTGCCGGTCCGCTA |
| 43 | GGTACATGCACTACGCGCACTCGG | CCGAGTGCGCGTAGTGCATGTACC |
| 44 | AATTCATCTCGGACTCCCGCGGTA | TACCGCGGGAGTCCGAGATGAATT |
| 45 | GCCAAATCTGGATTGGCAGGAATG | CATTCCTGCCAATCCAGATTTGGC |
| 46 | TGCATTTTCGGTTGAGGCACATCC | GGATGTGCCTCAACCGAAAATGCA |
| 47 | CCGCTCAATTCACCATGCTTCGCT | AGCGAAGCATGGTGAATTGAGCGG |
| 48 | CTCGGAAAGGTGCAACTTTGGTGT | ACACCAAAGTTGCACCTTTCCGAG |
| 49 | AATTCGACCAGCAGAACGTCCCAT | ATGGGACGTTCTGCTGGTCGAATT |
| 50 | GCCAGAGTCTCAACCTCACGGGAT | ATCCCGTGAGGTTGAGACTCTGGC |
| 51 | CCAACAACTGGAACGGGAACCCGC | GCGGGTTCCCGTTCCAGTTGTTGG |
| 52 | GAGAACTGATCGCTGAGGGGCATG | CATGCCCCTCAGCGATCAGTTCTC |
| 53 | GGCACACTAGACTTGTGGCACCGA | TCGGTGCCACAAGTCTAGTGTGCC |
| 54 | TCACATCCAAATATGGTCCGCGAA | TTCGCGGACCATATTTGGATGTGA |
| 55 | GTCTGCCGGTGTGACCGCTTCATT | AATGAAGCGGTCACACCGGCAGAC |
| 56 | CATCGCAGAGCATAAACACCCTCA | TGAGGGTGTTTATGCTCTGCGATG |
| 57 | GTTGGTATCTATGGCAGAGGCGGA | TCCGCCTCTGCCATAGATACCAAC |
| 58 | ACGAGGTGCCGCTGAGGTTCCATT | AATGGAACCTCAGCGGCACCTCGT |
| 59 | GGAATGAGTGGACCCAGGCACATT | AATGTGCCTGGGTCCACTCATTCC |
| 60 | TGTCAATATGCGTCCGTGTCGTCT | AGACGACACGGACGCATATTGACA |
| 61 | TGATGAGCCTCAGGGTACGAGGCA | TGCCTCGTACCCTGAGGCTCATCA |
| 62 | CACCGCGGTGTTCCTACAGAATGA | TCATTCTGTAGGAACACCGCGGTG |
| 63 | TTGTTGCCAATGGTGTCCGCTCGG | CCGAGCGGACACCATTGGCAACAA |
| 64 | TTAACCTGCGTCTGCCCCTTTCCT | AGGAAAGGGGCAGACGCAGGTTAA |
| 65 | AGGCGCGTTCCTGCCTTAGTGACG | CGTCACTAAGGCAGGAACGCGCCT |
| 66 | TAGGGCGATGGCACGAAGCTTCAA | TTGAAGCTTCGTGCCATCGCCCTA |
| 67 | TGCATAGAGCCAAAGTCGGCGATG | CATCGCCGACTTTGGCTCTATGCA |
| 68 | TTGAGAGGCAGGTGGCCACACGGA | TCCGTGTGGCCACCTGCCTCTCAA |
| 69 | TCCGCATTGTGAGAAAAAACGAGC | GCTCGTTTTTTCTCACAATGCGGA |
| 70 | GGCGGTTTCCGTAGCTATAGGTGC | GCACCTATAGCTACGGAAACCGCC |
| 71 | GGTGAAAATTTCGTAGCCACGGGC | GCCCGTGGCTACGAAATTTTCACC |
| 72 | CCGACGGAGGATGAAGACAATCAC | GTGATTGTCTTCATCCTCCGTCGG |
| 73 | CCAGTTTGGCCCAATTCGCCAAAA | TTTTGGCGAATTGGGCCAAACTGG |
| 74 | GGATCTATTAGGCCGTGCGCACAG | CTGTGCGCACGGCCTAATAGATCC |
| 75 | CGGATGTCACCGTTTGGACTTTCA | TGAAAGTCCAAACGGTGACATCCG |
| 76 | ATCGCAAATCCTGCTCGTCCCTAA | TTAGGGACGAGCAGGATTTGCGAT |
| 77 | CAGGGCATGCAATAATCGAGGTTC | GAACCTCGATTATTGCATGCCCTG |
| 78 | CATGCGTTGATATATGGGCCCAAG | CTTGGGCCCATATATCAACGCATG |
| 79 | CAGCTGCAGCTTGTGACCAACCAC | GTGGTTGGTCACAAGCTGCAGCTG |
| 80 | TTGTATGTCTGCCGACCGGCGACC | GGTCGCCGGTCGGCAGACATACAA |
| 81 | GATGGCGCCCGTTGATAGGTATGG | CCATACCTATCAACGGGCGCCATC |
| 82 | ATGAGAATCGCCGGCAATCTGCTA | TAGCAGATTGCCGGCGATTCTCAT |
| 83 | ATTTGCACTGACCGCAGGCTCGTG | CACGAGCCTGCGGTCAGTGCAAAT |
| 84 | CAGGGAGAACGGTTAAGTTCCCGT | ACGGGAACTTAACCGTTCTCCCTG |
| 85 | AGGCCGGCGATCGAGGAGTTTGGT | ACCAAACTCCTCGATCGCCGGCCT |
| 86 | ACACGGTGGTCTCTGATAGCGACC | GGTCGCTATCAGAGACCACCGTGT |
| 87 | GTGCAACGCCGAGGACTTCCATCA | TGATGGAAGTCCTCGGCGTTGCAC |
| 88 | TCGGTGCCTGATAGCCATTCCGAT | ATCGGAATGGCTATCAGGCACCGA |
| 89 | TGAAATACCACACAGCCAATTGGC | GCCAATTGGCTGTGTGGTATTTCA |
| 90 | GCATCGTGTACATGACTGCCGCGA | TCGCGGCAGTCATGTACACGATGC |
| 91 | CAGTGTTCTAACGGCGCGCGTGAA | TTCACGCGCGCCGTTAGAACACTG |
| 92 | CGCTTGCAACGTTGCACCTACTCT | AGAGTAGGTGCAACGTTGCAAGCG |
| 93 | CGAAAAACTAGTGGGCTCGCCGCG | CGCGGCGAGCCCACTAGTTTTTCG |
| 94 | CTTTCAGGGGAACTGCCGGAGTCG | CGACTCCGGCAGTTCCCCTGAAAG |
| 95 | TTGTGGCCTTCTTGTAAAGGCACG | CGTGCCTTTACAAGAAGGCCACAA |
| 96 | TCCACGAACGGCGACCCGTTGTCT | AGACAACGGGTCGCCGTTCGTGGA |
| 97 | CGACCTTGCACGAAACCTAACGAG | CTCGTTAGGTTTCGTGCAAGGTCG |
| 98 | GTGCAGCTTCACGAGCCAGCCTGA | TCAGGCTGGCTCGTGAAGCTGCAC |
| 99 | CGCTTTCGTGCGAATAGACGATGA | TCATCGTCTATTCGCACGAAAGCG |
| 100 | TGCGCTTACAGGCTCCTAGTGGTC | GACCACTAGGAGCCTGTAAGCGCA |
| 101 | CACGCGCTTAGTCGCGATCGCATA | TATGCGATCGCGACTAAGCGCGTG |
| 102 | CGGAGGGAGGGAGCTAGCCTTCGA | TCGAAGGCTAGCTCCCTCCCTCCG |
| 103 | GCATCCGGCCTGTTGATGACGCCT | AGGCGTCATCAACAGGCCGGATGC |
| 104 | AGGCCAATCGATCTTATTGCCGAG | CTCGGCAATAAGATCGATTGGCCT |
| 105 | CCTTCCAATGATTGCATACGCCCA | TGGGCGTATGCAATCATTGGAAGG |
| 106 | AACACTTGATCAGGCGGGTCGTCT | AGACGACCCGCCTGATCAAGTGTT |
| 107 | TGGAATCAAGGCCGTAAAGGACAG | CTGTCCTTTACGGCCTTGATTCCA |
| 108 | GCTCCCGTAACCTGTCCACCAGTG | CACTGGTGGACAGGTTACGGGAGC |
| 109 | AGTGGTGAATGGCCGCTACCCTGA | TCAGGGTAGCGGCCATTCACCACT |
| 110 | TGTTGAAGCGAGCTAAAACGGCCA | TGGCCGTTTTAGCTCGCTTCAACA |
| 111 | CAGCGCTCCAGAATTGACAGCAAT | ATTGCTGTCAATTCTGGAGCGCTG |
| 112 | AAGGTGGTGCCATTCATTTGGCTA | TAGCCAAATGAATGGCACCACCTT |
| 113 | CGTTAAACCGCAATCCGTTCGGCT | AGCCGAACGGATTGCGGTTTAACG |
| 114 | CACGAGATACCGGCGTAAGGGTGG | CCACCCTTACGCCGGTATCTCGTG |
| 115 | CTACGGCAAACGTGTGGAATGGGT | ACCCATTCCACACGTTTGCCGTAG |
| 116 | GTAGGGCGATGACGGGCGAACTAC | GTAGTTCGCCCGTCATCGCCCTAC |
| 117 | AATCGACCTCCGCACACATTCGCA | TGCGAATGTGTGCGGAGGTCGATT |
| 118 | GAGTCAGCATGGCGGCGGAGATTC | GAATCTCCGCCGCCATGCTGACTC |
| 119 | AGATAAAGACGCTGGCAACACGGG | CCCGTGTTGCCAGCGTCTTTATCT |
| 120 | GGTACCTCAACGCGAACCACTTGT | ACAAGTGGTTCGCGTTGAGGTACC |
| 121 | AAGCGATGGCTACCCAAGAGCGAT | ATCGCTCTTGGGTAGCCATCGCTT |
| 122 | AGAGCTTATGCAGAACCAGGCGCC | GGCGCCTGGTTCTGCATAAGCTCT |
| 123 | ATCGGTCTCACGCAGGGTTGGATA | TATCCAACCCTGCGTGAGACCGAT |
| 124 | TAGGTTGCCCGCCAGAAGAAACAT | ATGTTTCTTCTGGCGGGCAACCTA |
| 125 | CGGTGCTGTTGCAAAAGCCTGTAG | CTACAGGCTTTTGCAACAGCACCG |
| 126 | TGATGAAAGTTTGCGGCAGGACAC | GTGTCCTGCCGCAAACTTTCATCA |
| 127 | GTTGAGTGCAGGATGCAGCGATAG | CTATCGCTGCATCCTGCACTCAAC |
| 128 | AACATTGCGCGGTCCACCAGGGTT | AACCCTGGTGGACCGCGCAATGTT |
| 129 | GGGCAGTTAGAGAGGGCCAGAAGT | ACTTCTGGCCCTCTCTAACTGCCC |
| 130 | TCGAGCTGGTCCCCGTGAACGTGT | ACACGTTCACGGGGACCAGCTCGA |
| 131 | GTCTTGGGGGCCGCTTAGTGAAAA | TTTTCACTAAGCGGCCCCCAAGAC |
| 132 | ACTGTTGGCTTGCTCTCATGTCCA | TGGACATGAGAGCAAGCCAACAGT |
| 133 | AGGACCATTCGGAAGGCGAAGATA | TATCTTCGCCTTCCGAATGGTCCT |
| 134 | CTTGGGAGGCATCCGCTATAAGGA | TCCTTATAGCGGATGCCTCCCAAG |
| 135 | AATAAACGGAACGCACCGCTACAG | CTGTAGCGGTGCGTTCCGTTTATT |
| 136 | TTGTACGTGCGGTCCCCATAAGCA | TGCTTATGGGGACCGCACGTACAA |
| 137 | CGCACCAAACTGAGTTTCCCAGAC | GTCTGGGAAACTCAGTTTGGTGCG |
| 138 | ACCTGATCGTTCCCCTATTGGGAA | TTCCCAATAGGGGAACGATCAGGT |
| 139 | GGAACAGAGGCGAGGGGACTGAGC | GCTCAGTCCCCTCGCCTCTGTTCC |
| 140 | CCCTGCCTTGGCGTGTCGGCTTAT | ATAAGCCGACACGCCAAGGCAGGG |
| 141 | ACTCTGACACGCCAACTCCGGAAG | CTTCCGGAGTTGGCGTGTCAGAGT |
| 142 | CTGACGGTTTTCATTCGGCGTGCC | GGCACGCCGAATGAAAACCGTCAG |
| 143 | TGCGGTGGTTCATTGGAGCTGGCC | GGCCAGCTCCAATGAACCACCGCA |
| 144 | GCATGGCCAACTAGTGACTCGCAA | TTGCGAGTCACTAGTTGGCCATGC |
| 145 | AGGCCGTAAAGCGAATCTCACCTG | CAGGTGAGATTCGCTTTACGGCCT |
| 146 | CGAATATTATGCCGAGAATCCGCG | CGCGGATTCTCGGCATAATATTCG |
| 147 | ACAGACGAGCTCCCAACCACATGA | TCATGTGGTTGGGAGCTCGTCTGT |
| 148 | GGACGGTTTGTGCTGGATTGTCTG | CAGACAATCCAGCACAAACCGTCC |
| 149 | AAAGGCTATTGAGTTGGTTGGGCG | CGCCCAACCAACTCAATAGCCTTT |
| 150 | GATGGCCTATTCGGAGATCGGGCC | GGCCCGATCTCCGAATAGGCCATC |
| 151 | GATCCAGTAGGCAGCTTCATCCCA | TGGGATGAAGCTGCCTACTGGATC |
| 152 | AATAACTCGCGCGGGTATGCTTCT | AGAAGCATACCCGCGCGAGTTATT |
| 153 | GGAGGAGGTTTGTCTCGGAAAGCA | TGCTTTCCGAGACAAACCTCCTCC |
| 154 | CTTTGGTATGGCACATGCTGCCCG | CGGGCAGCATGTGCCATACCAAAG |
| 155 | AGAAAGGCTCGAGCAACGGGAACT | AGTTCCCGTTGCTCGAGCCTTTCT |
| 156 | AATCTACCGCACTGGTCCGCAAGT | ACTTGCGGACCAGTGCGGTAGATT |
| 157 | CGTGGCGGCCACAGTTTTTGGAGG | CCTCCAAAAACTGTGGCCGCCACG |
| 158 | TTGCAGTTCAATCCATACGCACGT | ACGTGCGTATGGATTGAAGTGCAA |
| 159 | GGCCCAAAGCCCCAGACCATTTTA | TAAAATGGTCTGGGGCTTTGGGCC |
| 160 | CGCCTGTCTTTGTCTCCGGACAAT | ATTGTCCGGAGACAAAGACAGGCG |
| 161 | TGAGGCAACAGGGGCCAAAAACTA | TAGTTTTTGGCCCCTGTTGCCTCA |
| 162 | AGCGGAAGTAGTCCTCGGCTCGTC | GACGAGCCGAGGACTACTTCCGCT |
| 163 | GGCCCCAAGGCTTAGAGATAGTGG | CCACTATCTCTAAGCCTTGGGGCC |
| 164 | GCACGTGAAGTTTAACCGCGATTC | GAATCGCGGTTAAACTTCACGTGC |
| 165 | AGCGGCAGAAACGTTCCTTGACGG | CCGTCAAGGAACGTTTCTGCCGCT |
| 166 | TCGTCGAGCAGACGAGATTGCACG | CGTGCAATCTCGTCTGCTCGACGA |
| 167 | TCTTTGCCGCGTAACTGACTGGTT | AAGCAGTCAGTTACGCGGCAAAGA |
| 168 | TTTATGTGCCAAGGGGTTAACCGA | TCGGTTAACCCCTTGGCACATAAA |
| 169 | TGTTACTGTGGTTCACGGCAGTCC | GGACTGCCGTGAACCACAGTAACA |
| 170 | CGCGCCTCGCTAGACCTTTTATTG | CAATAAAAGGTCTAGCGAGGCGCG |
| 171 | ACAAATGCGTGAGAGCTCCCAACT | AGTTGGGAGCTCTCACGCATTTGT |
| 172 | CGCGCAGATTATAGACCCGAATGT | ACATTCGGGTCTATAATCTGCGCG |
| 173 | CAAATAACGCCGCTGAATCGGCGT | ACGCCGATTCAGCGGCGTTATTTG |
| 174 | CCTTCGTGCATCGGTGATGATGTT | AACATCATCACCGATGCACGAAGG |
| 175 | TGAACACGAGCAACACTCCAACGC | GCGTTGGAGTGTTGCTCGTGTTCA |
| 176 | CAGCAGATCCTTCGTAGCGGTCGT | ACGACCGCTACGAAGGATCTGCTG |
| 177 | GGAACCTGGTGAGTTGTGCCTCAT | ATGAGGCACAACTCACCAGGTTCC |
| 178 | TCATAAGCGACAATCGCGGGCTTA | TAAGCCCGCGATTGTCGCTTATGA |
| 179 | CCCAACGTCACTGAAGCTCACAGT | ACTGTGAGCTTCAGTGACGTTGGG |
| 180 | TGTCAGAGCCCGCGACTCAGACGG | CCGTCTGAGTCGCGGGCTCTGACA |
| 181 | TACACGAAGCCTCTCCGTGGTCCA | TGGACCACGGAGAGGCTTCGTGTA |
| 182 | CTCAGAAGTCCTCGGCGAACTGGG | CCCAGTTCGCCGAGGACTTCTGAG |
| 183 | ATCCTTTTATCTACTCCGCGGCGA | TCGCCGCGGAGTAGATAAAAGGAT |
| 184 | AGGCGTGCAGCAACAGGATAAACC | GGTTTATCCTGTTGCTGCACGCCT |
| 185 | ACTCTCGAGGGAGTCTCTGGCACA | TGTGCCAGAGACTCCCTCGAGAGT |
| 186 | TTGCCAGGTCCATCGAGACCTGTT | AACAGGTCTCGATGGACCTGGCAA |
| 187 | TCCACTATAACTGCGGGTCCGTGT | ACACGGACCCGCAGTTATAGTGGA |
| 188 | GCCCAGTCGGCTCTAACAAGTTCG | CGAACTTGTTAGAGCCGACTGGGC |
| 189 | CGGAACGGATAATCGGCGTCAGGT | ACCTGACGCCGATTATCCGTTCCG |
| 190 | TAAAATAAGCGCCTGGCGGGAGGA | TCCTCCCGCCAGGCGCTTATTTTA |
| 191 | GCGCACTCGTGAAACCTTTCTCGC | GCGAGAAAGGTTTCACGAGTGCGC |
| 192 | AGTTTGCCAGGTACTGGCAAGTGC | GCACTTGCCAGTACCTGGCAAACT |
| 193 | ACAACGAGGGATGTCCAGCGGCAT | ATGCCGCTGGACATCCCTCGTTGT |
| 194 | TTCGCAGCACCCGCTAGGTACAGT | ACTGTACCTAGCGGGTGCTGCGAA |
| 195 | TAACCCGATTTTTGCGACTCTGCC | GGCAGAGTCGCAAAAATCGGGTTA |
| 196 | CGTCGCATTGCAAGCGTAGGCTTG | CAAGCCTACGCTTGCAATGCGACG |
| 197 | GAGCTGACGTCACCATCAGAGGAA | TTCCTCTGATGGTGACGTCAGCTC |
| 198 | GGAGGCTGGGGGTCGCGCTTAAGT | ACTTAAGCGCGACCCCCAGCCTCC |
| 199 | TTGTGGGAACCGCACTAGCTGGCT | AGCCAGCTAGTGCGGTTCCCACAA |
| 200 | CCCTCGCACTGTGTTCACCCTCTT | AAGAGGGTGAACACAGTGCGAGGG |
| 201 | TCATTGACTCGAATCCGCACAACG | CGTTGTGCGGATTCGAGTCAATGA |
| 202 | ACAGGGGTTGGCCTTCGTACGTAC | GTACGTACGAAGGCCAACCCCTGT |
| 203 | AGGCCGTGCAACATCACACAGGAT | ATCCTGTGTGATGTTGCACGGCCT |
| 204 | GGGCCGTGGTCACGTAATATTGGC | GCCAATATTACGTGACCACGGCCC |
| 205 | GCGCGGACATGAAACGACAAGGCC | GGCCTTGTCGTTTCATGTCCGCGC |
| 206 | CTTATTGGGTGCCGGTGTCGGATT | AATCCGACACCGGCACCCAATAAG |
| 207 | GGGGCGGTTACCAAAAAATCCGAT | ATCGGATTTTTTGGTAACCGCCCC |
| 208 | GCTAAAGCGTGCTCCGTAACTGCC | GGCAGTTACGGAGCACGCTTTAGC |
| 209 | ATCTCATGCATCTCGGTTCGTCGT | ACGACGAACCGAGATGCATGAGAT |
| 210 | ACGAAAAAAGTGTGCGGATCCCCT | AGGGGATCCGCACACTTTTTTCGT |
| 211 | CCAAGTACACCGCACGCATGTTTA | TAAACATGCGTGCGGTGTACTTGG |
| 212 | ATCGTGCGTGGAGTGTCGCATCTA | TAGATGCGACACTCCACGCACGAT |
| 213 | TCCAGATACCGCCCCGAACTTTGA | TCAAAGTTCGGGGCGGTATCTGGA |
| 214 | TCTGCTGGCAGCACGTGAAGTGGC | GCCACTTCACGTGCTGCCAGCAGA |
| 215 | TTGAAATTGCTCTGCCGTCAGTCA | TGACTGACGGCAGAGCAATTTCAA |
| 216 | AGTCAGGCGAGATGTTCAGGCAGC | GCTGCCTGAACATCTCGCCTGACT |
| 217 | ACAAGCCGACGTTAAGCCCGCCCA | TGGGCGGGCTTAACGTCGGCTTGT |
| 218 | CCCTAATGAGGCCAGTAACCTGCA | TGCAGGTTACTGGCCTCATTAGGG |
| 219 | GTGAGACACACATCCCCTCCAATG | CATTGGAGGGGATGTGTGTCTCAC |
| 220 | CGACGGATGCAGAGTTCAGTGGTC | GACCACTGAACTCTGCATCCGTCG |
| 221 | CCCGCATGCCTGGCGGTATTACAA | TTGTAATACCGCCAGGCATGCGGG |
| 222 | TTAGCAAAGCGGCGCCGTTAGCAA | TTGCTAACGGCGCCGCTTTGCTAA |
| 223 | CCCGACACGGGTCAGCGTAATAAT | ATTATTACGCTGACCCGTGTCGGG |
| 224 | GCGACGGCCCTGAGGTATGTCGTC | GACGACATACCTCAGGGCCGTCGC |
| 225 | CAAAAGTGTGTTCCCTTGCGCTTG | CAAGCGCAAGGGAACACACTTTTG |
| 226 | TCTCGAAGCACAGCCCGGTTATTG | CAATAACCGGGCTGTGCTTCGAGA |
| 227 | ATGCTAACCGTTGGCCATGGAACT | AGTTCCATGGCCAACGGTTAGCAT |
| 228 | CTTGCGGAGTGTTAGCCCAGCGGT | ACCGCTGGGCTAACACTCCGCAAG |
| 229 | TGCTCCCTAGGCGCTCGGAGGAGT | ACTCCTCCGAGCGCCTAGGGAGCA |
| 230 | CCAATGCCTTTGAGTAAGCGATGG | CCATCGCTTACTCAAAGGCATTGG |
| 231 | AGCAGATAACGTCCCAATGACGCC | GGCGTCATTGGGACGTTATCTGCT |
| 232 | TTGACCATTACGTGTTGCGCCCAT | ATGGGCGCAACACGTAATGGTCAA |
| 233 | TCGCGTATTTGCGGAATTCGTCTG | CAGACGAATTCCGCAAATACGCGA |
| 234 | CTGCGTGTCAACAATGTCCCGCAG | CTGCGGGACATTGTTGACACGCAG |
| 235 | TCTGGTGCCACGCAAGGTCCACAG | CTGTGGACCTTGCGTGGCACCAGA |
| 236 | CTCCGGGAGGTCACTTAATTGCGG | CCGCAATTAAGTGACCTCCCGGAG |
| 237 | TTTTCGTGATTGCCCGGAGGAGGC | GCCTCCTCCGGGCAATCACGAAAA |
| 238 | TCGGGATGTAGCTGGGGCTACCGG | CCGGTAGCCCCAGCTACATCCCGA |
| 239 | CGAGCCAACGCAAACACGTCCTTG | CAAGGACGTGTTTGCGTTGGCTCG |
| 240 | GCAAAGCCTTTGTGGGGCGGTAGT | ACTACCGCCCCACAAAGGCTTTGC |
| 241 | ATTCGACCGGAAATGAGGTCTTCG | CGAAGACCTCATTTCCGGTCGAAT |
| 242 | TTCGCTTGCTGAGTTGCTCTGTTC | GAACAGAGCAACTCAGCAAGCGAA |
| 243 | CGCGTGAAGACCCCATTCCCGAGT | ACTCGGGAATGGGGTCTTCACGCG |
| 244 | AACCGTATTCGCGGTCACTTGTGG | CCACAAGTGACCGCGAATACGGTT |
| 245 | GGGGCCAACCGTTTCGAGGCGTAT | ATACGCCTCGAAACGGTTGGCCCC |
| 246 | TTCGGCTGGCAGTCCAAACGGCTT | AAGCCGTTTGGACTGCCAGCCGAA |
| 247 | GGGTGTGGTTAGAATGCACGGTTC | GAACCGTGCATTCTAACCACACCC |
| 248 | GCGAGGACCGAACTAGACAAACGG | CCGTTTGTCTAGTTCGGTCCTCGC |
| 249 | ACGCACGCGTGACCGAAGTTGCTG | CAGCAACTTCGGTCACGCGTGCGT |
| 250 | TAAAAGGTCGCTTTGAAAGGGGGA | TCCCCCTTTCAAAGCGACCTTTTA |
| 251 | TGCGATCGCTAACTGCTGGGACAA | TTGTCCCAGCAGTTAGCGATCGCA |
| 252 | GGAGGTATAAGCGGAGCGGCCTCA | TGAGGCCGCTCCGCTTATACCTCC |
| 253 | ATGCTGACATGTCGTGCACCTCGT | ACGAGGTGCACGACATGTCAGCAT |
| 254 | TGTGGTTAAAGCGTCCGTTCAACG | CGTTGAACGGACGCTTTAACCACA |
| 255 | CGTTCACACCGGCGTAAGCTGCGT | ACGCAGCTTACGCCGGTGTGAACG |
| 256 | CCTATCCCGGCGAGAACTTCTGTG | CACAGAAGTTCTCGCCGGGATAGG |
| 257 | GTCTGCACTCACGCAGCGGAGGGA | TCCCTCCGCTGCGTGAGTGCAGAC |
| 258 | GCACGAGTTGGTGCTCGGCAGATT | AATCTGCCGAGCACCAACTCGTGC |
| 259 | AACGTCGCACGACACACGTTCGTC | GACGAACGTGTGTCGTGCGACGTT |
| 260 | ATGCGCGCTTATCCTAGCATGGTC | GACCATGCTAGGATAAGCGCGCAT |
| 261 | TCACGTTTTCGTCTCGACATGAGG | CCTCATGTCGAGACGAAAACGTGA |
| 262 | TGTGCCTCATCCTTAGGATACGGC | GCCGTATCCTAAGGATGAGGCACA |
| 263 | AGGTGGTGTGGGTCAACCGCTTTA | TAAAGCGGTTGACCCACACCACCT |
| 264 | CTGGATCGAAGGGACTGCAAGCTC | GAGCTTGCAGTCCCTTCGATCCAG |
| 265 | TAGATCAACTCGCGTACGCATGGA | TCCATGCGTACGCGAGTTGATCTA |
| 266 | GATCCTGCGGAGAAGAGAGTGCAG | CTGCACTCTCTTCTCCGCAGGATC |
| 267 | TACGTGTGGAGATGCCCCGAACCG | CGGTTCGGGGCATCTCCACACGTA |
| 268 | GCGCTATGTCAATCGTGGGCGTAG | CTACGCCCACGATTGACATAGCGC |
| 269 | AGCGAGGTTTCTAGCGTCGACACC | GGTGTCGACGCTAGAAACCTCGCT |
| 270 | ACCCAGGTTTTGCCGTTGTGGAAT | ATTCCACAACGGCAAAACCTGGGT |
| 271 | CCCTGTTAACGGCTGCGTAGTCTC | GAGACTACGCAGCCGTTAACAGGG |
| 272 | AGGCCGATTTCACCCGCCAATTGC | GCAATTGGCGGGTGAAATCGGCCT |
| 273 | GAGCCCTCACTCCTTGCCCTTTGA | TCAAAGGGCAAGGAGTGAGGGCTC |
| 274 | GGGTGGACATCCGCCTCGCAGTCA | TGACTGCGAGGCGGATGTCCACCC |
| 275 | GATGGCTGAGAACCGTGCTACGAT | ATCGTAGCACGGTTCTCAGCCATC |
| 276 | TCGACGTTAGGAGTGCTGCCAGAA | TTCTGGCAGCACTCCTAACGTCGA |
| 277 | CGAATGGGTCTGGACCTTGCATAG | CTATGCAAGGTCCAGACCCATTCG |
| 278 | GTGCACCAGACATTCGAACTCGGA | TCCGAGTTCGAATGTCTGGTGCAC |
| 279 | AGAGGCCCCGTATATCCCATCCAT | ATGGATGGGATATACGGGGCCTCT |
| 280 | AACGCCTGTTCAGAGCATCAGCGG | CCGCTGATGCTCTGAACAGGCGTT |
| 281 | AAGGCTCAACACGCCTATGTGCGC | GCGCACATAGGCGTGTTGAGCCTT |
| 282 | AGTCCGTGTTGCCAGATTGGCTCG | CGAGCCAATCTGGCAACACGGACT |
| 283 | ATGTCCCATGTAAAGACGCGTGTG | CACACGCGTCTTTACATGGGACAT |
| 284 | ATGGAGTCTGCTCACGCCCAAAGG | CCTTTGGGCGTGAGCAGACTCCAT |
| 285 | CGGCCTCCAACAAGGAGCACTAAC | GTTAGTGCTCCTTGTTGGAGGCCG |
| 286 | CAGAGCCGTGGCAACATTGCGAGC | GCTCGCAATGTTGCCACGGCTCTG |
| 287 | TCATTTGAATGAGGTGCGCACCGG | CCGGTGCGCACCTCATTCAAATGA |
| 288 | GACGTACCGGAAGCGCCGTATAAA | TTTATACGGCGCTTCCGGTACGTC |
| 289 | ATGCGAGCAATGGGATCCGGATTC | GAATCCGGATCCCATTGCTCGCAT |
| 290 | AGAGTGAGGCCTCCCTGACCAGTG | CACTGGTCAGGGAGGCCTCACTCT |
| 291 | CGCACCGTAAGTAGATTTGCCCGC | GCGGGCAAATCTACTTACGGTGCG |
| 292 | TGAACCTTTGAGCACGTCGTGCGC | GCGCACGACGTGCTCAAAGGTTCA |
| 293 | TCCGCCTTTTTGGTTACCTCGAAG | CTTCGAGGTAACCAAAAAGGCGGA |
| 294 | GAACGCCAACGGCACTAACACATC | GATGTGTTAGTGCCGTTGGCGTTC |
| 295 | CCGACAGCAGCCAAGACGTCCCAG | CTGGGACGTCTTGGCTGCTGTCGG |
| 296 | CATAAAAAAACCTGGGGCTCTGCG | CGCAGAGCCCCAGGTTTTTTTATG |
| 297 | TGCCAACTGTGCAGACCGGACTTA | TAAGTCCGGTCTGCACAGTTGGCA |
| 298 | GGCGAAAGAGCGAAACCGGCTCGT | ACGAGCCGGTTTCGCTCTTTCGCC |
| 299 | GGGATGCGTATTTTAGCGAACACG | CGTGTTCGCTAAAATACGCATCCC |
| 300 | TGGGATTCAGCGACCAGTACGCGA | TCGCGTACTGGTCGCTGAATCCCA |
| 301 | CCCGATATTCGCCCGGCCTATTCG | CGAATAGGCCGGGCGAATATCGGG |
| 302 | CGAGAAGATGCCTCACGCAACCAA | TTGGTTGCGTGAGGCATCTTCTCG |
| 303 | AACCTTGACCCGTGGATGACGCTA | TAGCGTCATCCACGGGTCAAGGTT |
| 304 | GGCTAGACGATGGATACCCGTGCC | GGCACGGGTATCCATCGTCTAGCC |
| 305 | GCCTCTTCTCGACGATGCGATTTT | AAAATCGCATCGTCGAGAAGAGGC |
| 306 | GCTTCCGGATGAACGGGATGGTTG | CAACCATCCCGTTCATCCGGAAGC |
| 307 | CCCTCCATGTTCTTCGAACGGTTT | AAACCGTTCGAAGAACATGGAGGG |
| 308 | TTGATGGGCGGCAATGCTCTTGCT | AGCAAGAGCATTGCCGCCCATCAA |
| 309 | ATTGTGAGATGCGCCAAATTCCCC | GGGGAATTTGGCGCATCTCACAAT |
| 310 | TCAGCACAGCCAGACGGTCAACTT | AAGTTGACCGTCTGGCTGTGCTGA |
| 311 | ACTCCACTCCTCGGTGGCAAACTA | TAGTTTGCCACCGAGGAGTGGAGT |
| 312 | TCTGGGCATGCCTGGACGGAGACG | CGTCTCCGTCCAGGCATGCCCAGA |
| 313 | TCTCAACTCCGGTACGACGAAACA | TGTTTCGTCGTACCGGAGTTGAGA |
| 314 | TTGCGTGGTCAAAGGCGCAACGTG | CACGTTGCGCCTTTGACCACGCAA |
| 315 | AGACAGCGATCCGCGGCTCATGAT | ATCATGAGCCGCGGATCGCTGTCT |
| 316 | CGCGTCTCTAACTGAGAGCAGCCA | TGGCTGCTCTCAGTTAGAGACGCG |
| 317 | AGGCGCACATGTACGGACATTCAG | CTGAATGTCCGTACATGTGCGCCT |
| 318 | GATGAGTGGCACGTCGGTGTGTAA | TTACACACCGACGTGCCACTCATC |
| 319 | TGATCCATATTGTCGGACGTTGCG | CGCAACGTCCGACAATATGGATCA |
| 320 | ACCTGCCGGGAGTTCATAGGCTAG | CTAGCCTATGAACTCCCGGCAGGT |
| 321 | AGCATTGGCGTTTTTCCGCAACGA | TCGTTGCGGAAAAACGCCAATGCT |
| 322 | GGTAATATTCAGCGCGACCGCTCA | TGAGCGGTCGCGCTGAATAYTACC |
| 323 | ATAGCGTACGACGAGGTGACGCGC | GCGCGTCACCTCGTCGTACGCTAT |
| 324 | TAGGTCACGATGCGTTTGACGCTA | TAGCGTCAAACGCATCGTGACCTA |
| 325 | ACTGCCCGTACCTCTGGTTCTGGC | GCCAGAACCAGAGGTACGGGCAGT |
| 326 | CCTTTGGCCTGAAGTTGTCGTAGC | GCTACGACAACTTCAGGCCAAAGG |
| 327 | GTGCCCCACGAGCGTATCGTTGTA | TACAACGATACGCTCGTGGGGCAC |
| 328 | AGGCGCTACGTGGGCCTGGAGCAA | TTGCTCCAGGCCCACGTAGCGCCT |
| 329 | GGGTGCTACCATTGCATTAGTCCG | CGGACTAATGCAATGGTAGCACCC |
| 330 | ACCACGCGCGTACGTGTAACCGAG | CTCGGTTACACGTACGCGCGTGGT |
| 331 | CCATGATGCATTGGGTGCATTTAG | CTAAATGCACCCAATGCATCATGG |
| 332 | GGTCCGGCCCTACGAAACGTTCGA | TCGAACGTTTCGTAGGGCCGGACC |
| 333 | CCGTGTGGCTGGAGATTCGTGTGA | TCACACGAATCTCCAGCCACACGG |
| 334 | GTTAGGGCGACGCATATTGGCACA | TGTGCCAATATGCGTCGCCCTAAC |
| 335 | GGGTCAGTCAGGTGCGTTAGGATC | GATCCTAACGCACCTGACTGACCC |
| 336 | GCCGTGAAGTCGAATGCAGATCGA | TCGATCTGCATTCGACTTCACGGC |
| 337 | GCCACCACCCAGTGCATTCAGGTA | TACCTGAATGCACTGGGTGGTGGC |
| 338 | GAGCTTAGTTTGCGGTCATCGGGC | GCCCGATGACCGCAAACTAAGCTC |
| 339 | TGTTTGCCGCCATTAGGGAGTAAC | GTTACTCCCTAATGGCGGCAAACA |
| 340 | GCTCCGCTGGATGTGCCGGTTTAG | CTAAACCGGCACATCCAGCGGAGC |
| 341 | CGGTAGCATGCGAGATCCCTGTTA | TAACAGGGATCTCGCATGCTACCG |
| 342 | CTACGCTCTACCAGTTGCCTGCGA | TCGCAGGCAACTGGTAGAGCGTAG |
| 343 | GTGCCTCCTGCTGTATTTGCCAAG | CTTGGCAAATACAGCAGGAGGCAC |
| 344 | TTGCGACTCGACTTGGACGAGTAG | CTACTCGTCCAAGTCGAGTCGCAA |
| 345 | TCTGGGAGCTGTTTACTCCAGCCA | TGGCTGGAGTAAACAGCTCCCAGA |
| 346 | TGCACGCGGAACTCCCTTTACCAT | ATGGTAAAGGGAGTTCCGCGTGCA |
| 347 | TGGCAGCAAATGAATCGAAAGCAC | GTGCTTTCGATTCATTTGCTGCCA |
| 348 | AACTGGTGACGCGGTACAGCGAAG | CTTCGCTGTACCGCGTCACCAGTT |
| 349 | AGACGATTACGCTGGACGCCGTCG | CGACGGCGTCCAGCGTAATCGTCT |
| 350 | ATGCCCTCCTTCATGGAAAGGGTT | AACCCTTTCCATGAAGGAGGGCAT |
| 351 | ATTCTCGGAGCGTATGCGCCAGAA | TTCTGGCGCATACGCTCCGAGAAT |
| 352 | ATAGCGGAGTTTGGGTACGCGAAC | GTTCGCGTACCCAAACTCCGCTAT |
| 353 | ACCTACGCATACCGCTTGGCGAGG | CCTCGCCAAGCGGTATGCGTAGGT |
| 354 | GATTACCTGAATGGCCAAGCGAGC | GCTCGCTTGGCCATTCAGGTAATC |
| 355 | CCTGTTAGCATCACGGCGCTTAGG | CCTAAGCGCCGTGATGCTAACAGG |
| 356 | CGGAATGATGCGCTCGACAACGCT | AGCGTTGTCGAGCGCATCATTCCG |
| 357 | TGAGAGAGGCGTTGGTTAAGGCAA | TTGCCTTAACCAACGCCTCTCTCA |
| 358 | AAGCAGGCGAAGGGATACTCCTCG | CGAGGAGTATCCCTTCGCCTGCTT |
| 359 | TCACGACAGACGGGCCGAGATTAC | GTAATCTCGGCCCGTCTGTCGTGA |
| 360 | AAGCAATTTGGCCTCGTTTTGTGA | TCACAAAACGAGGCCAAATTGCTT |
| 361 | GCTGGTTGCGGTAGGATCGCATAT | ATATGCGATCCTACCGCAACCAGC |
| 362 | TTGTGAATCCGTTCTGTCCCCGAC | GTCGGGGACAGAACGGATTCACAA |
| 363 | TGGGCTCCTCTGAGGCGAGATGGC | GCCATCTCGCCTCAGAGGAGCCCA |
| 364 | GGATAGAGTGAATCGACCGGCAAC | GTTGCCGGTCGATTCACTCTATCC |
| 365 | TGCACCGAACGTGCACGAGTAATT | AATTACTCGTGCACGTTCGGTGCA |
| 366 | GCCAGTATTCTCGGGTGTTGGACG | CGTCCAACACCCGAGAATACTGGC |
| 367 | TCGCTACCTAAGACCGGGCCATAC | GTATGGCCCGGTCTTAGGTAGCGA |
| 368 | TGGCATTGACGAGCAGCAGTCAGT | ACTGACTGCTGCTCGTCAATGCCA |
| 369 | CGCGTCCCAGCGCCCTTGGAGTAT | ATACTCCAAGGGCGCTGGGACGCG |
| 370 | ATGAAGCCTACCGGGCGACTTCGT | ACGAAGTCGCCCGGTAGGCTTCAT |
| 371 | CCAGACAGATGGCCTGGAACCATG | CATGGTTCCAGGCCATCTGTCTGG |
| 372 | TGGCGTGGGACCATCTCAAAGCTA | TAGCTTTGAGATGGTCCCACGCCA |
| 373 | CCGCATGGGAACACGTGTCAAGGT | ACCTTGACACGTGTTCCCATGCGG |
| 374 | GCCCACTCGTCAGCTGGACGTAAT | ATTACGTCCAGCTGACGAGTGGGC |
| 375 | ATTACGGTCGTGATCCAGAAAGCG | CGCTTTCTGGATCACGACCGTAAT |
| 376 | TGCGAGGTGAGCACCTACGAGAGA | TCTCTCGTAGGTGCTCACCTCGCA |
| 377 | GGGCCGCATTCTTGATGTCCATTC | GAATGGACATCAAGAATGCGGCCC |
| 378 | CCTCGGATGTGGGCTCTCGCCTAG | CTAGGCGAGAGCCCACATCCGAGG |
| 379 | TAGGCATGTTGGCGTGAGCGCTAT | ATAGCGCTCACGCCAACATGCCTA |
| 380 | CGATACGAACGAGGATGTCCGCCT | AGGCGGACATCCTCGTTCGTATCG |
| 381 | TACGCCGGTTAGCACGGTGCGCTA | TAGCGCACCGTGCTAACCGGCGTA |
| 382 | CATACGATGTCCGGGCCGTGTCGC | GCGACACGGCCCGGACATCGTATG |
| 383 | ATCCGCAGTTGTATGGCGCGTTAT | ATAACGCGCCATACAACTGCGGAT |
| 384 | GGGTAAGGGACAAAGATGGGATGG | CCATCCCATCTTTGTCCCTTACCC |
| 385 | ATTGGAGTGTTTTGGTGAATCCGC | GCGGATTCACCAAAACACTCCAAT |
| 386 | GAACCGAGCCAACGTATGGACACG | CGTGTCCATACGTTGGCTCGGTTC |
| 387 | GCCGTCAAGCTTAAGGTTTTGGGC | GCCCAAAACCTTAAGCTTGACGGC |
| 388 | ACCTGCTTTTGGGTGGGTGATATG | CATATCACCCACCCAAAAGCAGGT |
| 389 | AATCGTGGGCGCAGCAAACGTATA | TATACGTTTGCTGCGCCCACGATT |
| 390 | GTCGCCGGATTGCTCAGTATAAGC | GCTTATACTGAGCAATCCGGCGAC |
| 391 | ACCCGTCGATGCTTCCTCCTCAGA | TCTGAGGAGGAAGCATCGACGGGT |
| 392 | ATCCGGGTGGGCGATACAAGAGAT | ATCTCTTGTATCGCCCACCCGGAT |
| 393 | TTCCGCATGAGTCAGCTTTGAAAA | TTTTCAAAGCTGACTCATGCGGAA |
| 394 | GCAAAGTCCCACTGGCAAGCCGAT | ATCGGCTTGCCAGTGGGACTTTGC |
| 395 | CGACCTCGGCTTCATCGTACACAT | ATGTGTACGATGAAGCCGAGGTCG |
| 396 | CTCATGAGCGCAGTTGTGCGTGAG | CTCACGCACAACTGCGCTCATGAG |
| 397 | CAGATGAAGGATCCACGGCCGGAG | CTCCGGCCGTGGATCCTTCATCTG |
| 398 | TCAAAGGCTCTTGGATACAGCCGT | ACGGCTGTATCCAAGAGCCTTTGA |
| 399 | TCCGCTAATTTCCAATCAGGGCTC | GAGCCCTGATTGGAAATTAGCGGA |
| 400 | ACGCACGGCGCTTTTGCCTTAATG | CATTAAGGCAAAAGCGCCGTGCGT |
| 401 | TGACAACGTCACAAGGAGCAGGAC | GTCCTGCTCCTTGTGACGTTGTCA |
| 402 | CTTAGTTGGGGCGCGGTATCCAGA | TCTGGATACCGCGCCCCAACTAAG |
| 403 | GCTCTAATGCCGTGGAGTCGGAAC | GTTCCGACTCCACGGCATTAGAGC |
| 404 | CCGATTACAAATTGACTGACCGCA | TGCGGTCAGTCAATTTGTAATCGG |
| 405 | AGACGTACGTGAGCCTCCCGTGTC | GACACGGGAGGCTCACGTACGTCT |
| 406 | AATGGAGCGATACGATCCAACGCA | TGCGTTGGATCGTATCGCTCCATT |
| 407 | GGAGGCGCTGTACTGATAGGCGTA | TACGCCTATCAGTACAGCGCCTCC |
| 408 | TGTTTTTGAATTGACCACACGGGA | TCCCGTGTGGTCAATTCAAAAACA |
| 409 | CATGTCTGGATGCGCTCAATGAAG | CTTCATTGAGCGCATCCAGACATG |
| 410 | GCCCGCTAATCCGACACCCAGTTT | AAACTGGGTGTCGGATTAGCGGGC |
| 411 | CCATTGACAGGAGAGCCATGAGCC | GGCTCATGGCTCTCCTGTCAATGG |
| 412 | GAATCACCGAATCACCGACTCGTT | AACGAGTCGGTGATTCGGTGATTC |
| 413 | AACCAGCCGCAGTAGCTTACGTCG | CGACGTAAGCTACTGCGGCTGGTT |
| 414 | TTTTCTGAGGGACACGCGGGCGTT | AACGCCCGCGTGTCCCTCAGAAAA |
| 415 | GGTGCTCCGTTTGATCGATCCTCC | GGAGGATCGATCAAACGGAGCACC |
| 416 | CCGCTTAGGCCATACTCTGAGCCA | TGGCTCAGAGTATGGCCTAAGCGG |
| 417 | TAAGACATACCGACGCCCTTGCCT | AGGCAAGGGCGTCGGTATGTCTTA |
| 418 | GTTCCCGACGCCAGTCATTGAGAC | GTCTCAATGACTGGCGTCGGGAAC |
| 419 | TAAAAGTTTCGCGGAGGTCGGGCT | AGCCCGACCTCCGCGAAACTTTTA |
| 420 | CGGTCCAGACGAGCTGAGTTCGGC | GCCGAACTCAGCTCGTCTGGACCG |
| 421 | CGGCGTAGCGGCTACGGACTTAAA | TTTAAGTCCGTAGCCGCTACGCCG |
| 422 | GCTTGGATGCCCATGCGGCAAGGT | ACCTTGCCGCATGGGCATCCAAGC |
| 423 | AGCGGGATCCCAGAGTTTCGAAAA | TTTTCGAAACTCTGGGATCCCGCT |
| 424 | GAGCTTGAGAGCGAGGTCATCCTC | GAGGATGACCTCGCTCTCAAGGTC |
| 425 | GCATCGGCCGTTTTGACCATATTC | GAATATGGTCAAAACGGCCGATGC |
| 426 | CATAGCGCTGCACGTTTCGACCGC | GCGGTCGAAACGTGCAGCGCTATG |
| 427 | ACCCGACAACCACCAATTCAAAAA | TTTTTGAATTGGTGGTTGTCGGGT |
| 428 | GCGAACACTCATAAGAGCGCCCTG | CAGGGCGCTCTTATGAGTGTTCGC |
| 429 | CCGCCGAGTGTAGAGAGACTCCGA | TCGGAGTCTCTCTACACTCGGCGG |
| 430 | GACATCGGGAGCCGGAAACATGAG | CTCATGTTTCCGGCTCCCGATGTC |
| 431 | TCGTGTAGACTCGGCGACAGGCGT | ACGCCTGTCGCCGAGTCTACACGA |
| 432 | ATGCGCATATACTGACTGCGCAGG | CCTGCGCAGTCAGTATATGCGCAT |
| 433 | ACAAGCGAACCCGAGTTTTGATGA | TCATCAAAACTCGGGTTCGCTTGT |
| 434 | GCATGAGACTCCGCGAAGACATGT | ACATGTCTTCGCGGAGTCTCATGC |
| 435 | TCCTACATGTCGCGTCACGATCAC | GTGATCGTGACGCGACATGTAGGA |
| 436 | GACCGATCGCGAAGTCGTACACAT | ATGTGTACGACTTCGCGATCGGTC |
| 437 | GTCGCCAGGACTGGGCCGATGTGA | TCACATCGGCCCAGTCCTGGCGAC |
| 438 | ACCGATAAGACTTGCATCCGAACG | CGTTCGGATGCAAGTCTTATCGGT |
| 439 | TCCATAACCAGTCCGAAGTGCCGG | CCGGCACTTCGGACTGGTTATGGA |
| 440 | ACGCGCCCTGCATCTCGTATTTAA | TTAAATACGAGATGCAGGGCGCGT |
| 441 | AGACCGCATCAATTGGCGCGTACC | GGTACGCGCCAATTGATGCGGTCT |
| 442 | AGAGGCTTGGCAAGTAGGGACCCT | AGGGTCCCTACTTGCCAAGCCTCT |
| 443 | GCAATGGACGCCAGACGATACCGG | CCGGTATCGTCTGGCGTCCATTGC |
| 444 | GCTGGACTTAGTCGTGTTCGGCGG | CCGCCGAACACGACTAAGTCCAGC |
| 445 | AGGCATCGTGCCGGATTGCTCCCT | AGGGAGCAATCCGGCACGATGCCT |
| 446 | TGCGCATGTCGACGTTGAACAAAG | CTTTGTTCAACGTCGACATGCGCA |
| 447 | TTCGGGTCACATCCGATGCCATAC | GTATGGCATCGGATGTGACCCGAA |
| 448 | ACCCATCGCCGGAAAGCGATGTTG | CAACATCGCTTTCCGGCGATGGGT |
| 449 | AAGCGCTGACTCGGCTAAGAATCA | TGATTCTTAGCCGAGTCAGCGCTT |
| 450 | ACTTCCAAGTCCTTGACCGTCCGA | TCGGACGGTCAAGGACTTGGAAGT |
| 451 | TCTCAATATTCCCGTAGTCGCCCA | TGGGCGACTACGGGAATATTGAGA |
| 452 | AACAGTTCCTCTTTTTCCTGGCGC | GCGCCAGGAAAAAGAGGAACTGTT |
| 453 | CGTCCTCCATGTTGTCACGAACAG | CTGTTCGTGACAACATGGAGGACG |
| 454 | TGCGCAGACCTACCTGTCTTTGCT | AGCAAAGACAGGTAGGTCTGCGCA |
| 455 | ATGGACGGCTTCGCAGTCCTCCTT | AAGGAGGACTGCGAAGCCGTCCAT |
| 456 | TGAACGCTTTCTATGGGCCACGTA | TACGTGGCCCATAGAAAGCGTTCA |
| 457 | TGAACCCTGCCGCGAGCGATAACC | GGTTATCGCTCGCGGCAGGGTTCA |
| 458 | GTTCTTGCGCGATGAATCAGGACC | GGTCCTGATTCATCGCGCAAGAAC |
| 459 | AGGGTACGTGTCGCAGCTTCGCGT | ACGCGAAGCTGCGACACGTACCCT |
| 460 | ACCCTTGCTCCGCCATGTCTCTCA | TGAGAGACATGGCGGAGCAAGGGT |
| 461 | GGGACAAGGATTGAAGCTGGCGTC | GACGCCAGCTTCAATCCTTGTCCC |
| 462 | TGTCGTTGCTCCCGAGTACCATTG | CAATGGTACTCGGGAGCAACGACA |
| 463 | GTTGTCCGAGACGTTTGTGTCAGC | GCTGACACAAACGTCTCGGACAAC |
| 464 | GCTGGTGAACACTCACGAACCGCT | AGCGGTTCGTGAGTGTTCACCAGC |
| 465 | GCAGACAGGGCAAATCGGTGCAAA | TTTGCACCGATTTGCCCTGTCTGC |
| 466 | CCCATCACAACGAGTGGCGACTTT | AAAGTCGCCACTCGTTGTGATGGG |
| 467 | GCTTCTACAGCTGGCGTGCTAGCG | CGCTAGCACGCCAGCTGTAGAAGC |
| 468 | GAATGTGTGCCGACCATTCTAGCC | GGCTAGAATGGTCGGCACACATTC |
| 469 | CCAGCGGAAGTTAGAGCTCTGTGG | CCACAGAGCTCTAACTTCCGCTGG |
| 470 | TTTTTACCGACCACTCCATGTCGG | CCGACATGGAGTGGTCGGTAAAAA |
| 471 | GCGGCTATGTGATGACGGCCTAGC | GCTAGGCCGTCATCACATAGCCGC |
| 472 | AGTACACGGGCGTGTTAGCGCTCC | GGAGCGCTAACACGCCCGTGTACT |
| 473 | TCCTGTGTGGTGGCGCACTCCCAC | GTGGGAGTGCGCCACCACACAGGA |
| 474 | CCAACTAACCAATCGCGCGGATGA | TCATCCGCGCGATTGGTTAGTTGG |
| 475 | AGTGAGTGACCAAGGCAGGAGCAA | TTGCTCCTGCCTTGGTCACTCACT |
| 476 | CATCTTTCGCGGAGTTTATTGCGG | CCGCAATAAACTCCGCGAAAGATG |
| 477 | CTTCGTCCGGTTAGTGCGACAGCA | TGCTGTCGCACTAACCGGACGAAG |
| 478 | CTCACGAAAACGTGGGCCCGAAAT | ATTTCGGGCCCACGTTTTCGTGAG |
| 479 | CGCAGCAGCTGAACTCTAGCATTG | CAATGCTAGAGTTCAGCTGCTGCG |
| 480 | AGGAGACATACGCCCAAATGGTGC | GCACCATTTGGGCGTATGTCTCCT |
| 481 | ATTGAGAACTCGTGCGGGAGTTTG | CAAACTCCCGCACGAGTTCTCAAT |
| 482 | CTCTTTGTAGGCCCAGGAGGAGCA | TGCTCCTCCTGGGCCTACAAAGAG |
| 483 | GCCGCAGGGTCGATAATTGGTCTA | TAGACCAATTATCGACCCTGCGGC |
| 484 | AAACGCCGCCCTGAGACTATTGGG | CCCAATAGTCTCAGGGCGGCGTTT |
| 485 | CTGAGTTGCCTGGAACGTTGGACT | AGTCCAACGTTCCAGGCAACTCAG |
| 486 | CGGATGGGTTGCAGAGTATGGGAT | ATCCCATACTCTGCAACCCATCCG |
| 487 | CTGACCTTTGGGGGTTAGTGCGGT | ACCGCACTAACCCCCAAAGGTCAG |
| 488 | GGAAATGAGAACCTTACCCCAGCG | CGCTGGGGTAAGGTTCTCATTTCC |
| 489 | AACGCATCGTCCGTCAACTCATCA | TGATGAGTTGACGGACGATGCGTT |
| 490 | TGGAGAGAGACTTCGGCCATTGTT | AACAATGGCCGAAGTCTCTCTCCA |
| 491 | TTGCGCTCATTGGATCTTGTCAGG | CCTGACAAGATCCAATGAGCGCAA |
| 492 | AGCGCGTTAAAGCACGGCAACATT | AATGTTGCCGTGCTTTAACGCGCT |
| 493 | AGCCAGTAAACTGTGGGCGGCTGT | ACAGCCGCCCACAGTTTACTGGCT |
| 494 | CGACTGATGTGCAACCAGCAGCTG | CAGCTGCTGGTTGCACATCAGTCG |
| 495 | GGTTGCTCATACGACGAGCGAGTG | CACTCGCTCGTCGTATGAGCAACC |
| 496 | GCGCAAATCCACGGAACCCGTACC | GGTACGGGTTCCGTGGATTTGCGC |
| 497 | ACGCAGTTTATTCCCCTGGCTTCT | AGAAGCCAGGGGAATAAACTGCGT |
| 498 | AGAACCTCCGCGCCTCCGTAGTAG | CTACTACGGAGGCGCGGAGGTTCT |
| 499 | AAAGGAGCTTTCGCCCAACGTACC | GGTACGTTGGGCGAAAGCTCCTTT |
| 500 | AGTGATTGTGCCACTCCACAGCTC | GAGCTGTGGAGTGGCACAATCACT |
| 501 | GCGATCGTCGAGGGTTGAGCTGAA | TTCAGCTCAACCCTCGACGATCGC |
| 502 | GGGAGACAGCCATTATGGTCCTCG | CGAGGACCATAATGGCTGTCTCCC |
| 503 | GAGACGCTGTCACTCCGGCAGAAC | GTTCTGCCGGAGTGACAGCGTCTC |
| 504 | CCACCGGTCGCTTAAGATGCACTT | AAGTGCATCTTAAGCGACCGGTGG |
| 505 | CGGCATAACGTCCAGTCCTGGGAC | GTCCCAGGACTGGACGTTATGCCG |
| 506 | AAGCGGAACGGGTTATACCGAGGT | ACCTCGGTATAACCCGTTCCGCTT |
| 507 | TGCACACTAGGTCCGTCGCTTGAT | ATCAAGCGACGGACCTAGTGTGCA |
| 508 | AGGGAACCGCGTTCAAACTCAGTT | AACTGAGTTTGAACGCGGTTCCCT |
| 509 | GAATTACAACCACCCGCTCGTGTT | AACACGAGCGGGTGGTTGTAATTC |
| 510 | TTCAGTGCTCACGAAGCATGGATT | AATCCATGCTTCGTGAGCACTGAA |
| 511 | TTAGTTTGGCGTTGGGACTTCACC | GGTGAAGTCCCAACGCCAAACTAA |
| 512 | AATGCGACCTCGACGAGCCTCATA | TATGAGGCTCGTCGAGGTCGCATT |
| 513 | CCGAAACCGTTAACGTGGCGCACA | TGTGCGCCACGTTAACGGTTTCGG |
| 514 | TAAAGTAACAAGGCGACCTCCCGC | GCGGGAGGTCGCCTTGTTACTTTA |
| 515 | TAATGATTTTAGTCGCGGGGTGGG | CCCACCCCGCGACTAAAATCATTA |
| 516 | GGCTACTCTAAGTGCCCGCTCAGG | CCTGAGCGGGCACTTAGAGTAGCC |
| 517 | TGGCGGACGACTCAATATCTCACG | CGTGAGATATTGAGTCGTCCGCCA |
| 518 | GGGCGTTAGGCGTAATAGACCGTC | GACGGTCTATTACGCCTAACGCCC |
| 519 | GCCACCTTTAGACGGCGGCTCTAG | CTAGAGCCGCCGTCTAAAGGTGGC |
| 520 | GAGATGTGTAAACGTGCAGGCACC | GGTGCCTGCACGTTTACACATCTC |
| 521 | TAGCTCGTGGCCCTCCAAGCGTGT | ACACGCTTGGAGGGCCACGAGCTA |
| 522 | GTGTCGGCGCTATTTGGCCTTACC | GGTAAGGCCAAATAGCGCCGACAC |
| 523 | CCAGGGAAGCAACTGGTTGCCATT | AATGGCAACCAGTTGCTTCCCTGG |
| 524 | TTCCGAAACTAAGCCAGAACCGCT | AGCGGTTCTGGCTTAGTTTCGGAA |
| 525 | GCAAACCCGGTAACCCGAGAGTTC | GAACTCTCGGGTTACCGGGTTTGC |
| 526 | GCAAATGGCGTCATGCACGAACGT | ACGTTCGTGCATGACGCCATTTGC |
| 527 | AGTACTTTCGCGCCCAGTTTAGGG | CCCTAAACTGGGCGCGAAAGTACT |
| 528 | AAGATCTGCGAGGCATCCCGGCTT | AAGCCGGGATGCCTCGCAGATCTT |
| 529 | GCAAGTGTATCGCACAGTGCGATT | AATCGCACTGTGCGATACACTTGC |
| 530 | CCGACAAGGCCTCAATTCATTCTG | CAGAATGAATTGAGGCCTTGTCGG |
| 531 | GTCTCGTCTCAACTTTAAGGCGCG | CGCGCCTTAAAGTTGAGACGAGAC |
| 532 | ATCCAGAGATCCGTTTTGCAGCGT | ACGCTGCAAAACGGATCTCTGGAT |
| 533 | GTCACCAGGAGGGAAGTTTCACCC | GGGTGAAACTTCCCTCCTGGTGAC |
| 534 | TTCCGTCAGGCGGATCAACGGAAT | ATTCCGTTGATCCGCCTGACGGAA |
| 535 | ATGCCGGACACGCATTACACAGGC | GCCTGTGTAATGCGTGTCCGGCAT |
| 536 | TGGGCCGCTTGGCGCTTTCATAGA | TCTATGAAAGCGCCAAGCGGCCCA |
| 537 | CCTAGCGCGAGCTTTACTGACCAG | CTGGTCAGTAAAGCTCGCGCTAGG |
| 538 | TTGGCCAGGAATATGGTCTCGAGA | TCTCGAGACCATATTCCTGGCCAA |
| 539 | GTCTGCGGCCGACTTGCTATGCAT | ATGCATAGCAAGTCGGCCGCAGAC |
| 540 | AACTTGCTCATTCTCAAGCCGACG | CGTCGGCTTGAGAATGAGCAAGTT |
| 541 | ACGTCAGCGATTGTGGCGAAATAT | ATATTTCGCCACAATCGCTGACGT |
| 542 | ACGGCCTGCGTCAGCACATGCATC | GATGCATGTGCTGACGCAGGCCGT |
| 543 | ATACCTCCGCAGAACCATTCCGTT | AACGGAATGGTTCTGCGGAGGTAT |
| 544 | AGTTCGCGGTCCCACGATTCACTT | AAGTGAATCGTGGGACCGCGAACT |
| 545 | TGCTCAATTTGTGCAGAAAACGCC | GGCGTTTTCTGCACAAATTGAGCA |
| 546 | TTATCGCGAGAGACGACCGTGTCC | GGACACGGTCGTCTCTCGCGATAA |
| 547 | GACGCGACGTGAGTAGTGGAAGCG | CGCTTCCACTACTCACGTCGCGTC |
| 548 | ATGGTAGGGGCATTGGGCTTTCCT | AGGAAAGCCCAATGCCCCTACCAT |
| 549 | CCAAATATAGCCGCGCGGAGACAT | ATGTCTCCGCGCGGCTATATTTGG |
| 550 | GCAAACCCTGATTGAATCGTGCCC | GGGCACGATTCAATCAGGGTTTGC |
| 551 | TAGCGTCTTGCGTGAAACCATGGG | CCCATGGTTTCACGCAAGACGCTA |
| 552 | CCACCCCGACAGCGCTGGACTCTT | AAGAGTCCAGCGCTGTCGGGGTGG |
| 553 | ACGAGCACTGAAGGCTGCTTTACG | CGTAAAGCAGCCTTCAGTGCTCGT |
| 554 | CATATCAGCGTCGTCTAGCTCGCG | CGCGAGCTAGACGACGCTGATATG |
| 555 | TGATCCCGGACCGGCTAGACTAAT | ATTAGTCTAGCCGGTCCGGGATCA |
| 556 | GGCCCCGACACTACAGGGTAATCA | TGATTACCCTGTAGTGTCGGGGCC |
| 557 | GGCTCCAGGGCGAGATTATGAATG | CATTCATAATCTCGCCCTGGAGCC |
| 558 | CAAAATCCGATGGGCGGAAAATTA | TAATTTTCCGCCCATCGGATTTTG |
| 559 | CACAGGCGCATAGGGAGCAAGCTA | TAGCTTGCTCCCTATGCGCCTGTG |
| 560 | TAGCTATTTGCCCCGATGGGCTAC | AGTAGCCCATCGGGGCAATAGCTA |
| 561 | TGGTACGCGGTCCATAGCAAGTCG | CGACTTGCTATGGACCGCGTACCA |
| 562 | GACGCTGTGGCTCGGAAACTGTTC | GAACAGTTTCCGAGCCACAGCGTC |
| 563 | CCTGGGTTCGCCGCGTGGTAACTG | CAGTTACCACGCGGCGAACCCAGG |
| 564 | TTCCCGCGTAGCCCAACAGCTATA | TATAGCTGTTGGGCTACGCGGGAA |
| 565 | TTCGCGGATTGCTGCCGCATAACA | TGTTATGCGGCAGCAATCCGCGAA |
| 566 | AAAAATGGCACCGAAGTTGAGGCA | TGCCTCAACTTCGGTGCCATTTTT |
| 567 | CATTCCGCGCGAGTTGAAATCCAG | CTGGATTTCAACTCGCGCGGAATG |
| 568 | ACGCACGTTTTTTGGCACGGTTAA | TTAACCGTGCCAAAAAACGTGCGT |
| 569 | TGTCCATGACGTCGTTTCTCTGGT | ACCAGAGAAACGACGTCATGGACA |
| 570 | TCTCAGTCGGACTCGTATGCCAGA | TCTGGCATACGAGTCCGACTGAGA |
| 571 | CTCCAAACGCACACATCAAGCATC | GATGCTTGATGTGTGCGTTTGGAG |
| 572 | TTCAACCAAGGGGGGTGTTCGTGA | TCACGAACACCCCGCTTGGTTGAA |
| 573 | GGTGTCGGAGGGTGGTGACCTCGA | TCGAGGTCACCACCCTCCGACACC |
| 574 | AGCGCTTTTGGTCATGATTTGCAA | TTGCAAATCATGACCAAAAGCGCT |
| 575 | CCGAGGACTTACGTCTGCCCAGGA | TCCTGGGCAGACGTAAGTCCTCGG |
| 576 | GCCCAATCCAGTTCTTATGCGCCC | GGGCGCATAAGAACTGGATTGGGC |
| 577 | CGGGTTAACCCACGCAAGTTATGA | TCATAACTTGCGTGGGTTAACCCG |
| 578 | TGATTAGCGCTCAATACACGCGTG | CACGCGTGTATTGAGCGCTAATCA |
| 579 | AAGGGCAGACCTTTGGTTCGACTG | CAGTCGAACCAAAGGTCTGCCCTT |
| 580 | GCGCCACAAGATTCACATGTCATT | AATGACATGTGAATCTTGTGGCGC |
| 581 | GCCATGTTCAAGGGCCTTTCGAAG | CTTCGAAAGGCCCTTGAACATGGC |
| 582 | CGCGGTGTTTTGTCTAGGTGCCGG | CCGGCACCTAGACAAAACACCGCG |
| 583 | CAACATTGTGGTGGCACTCCATCC | GGATGGAGTGCCACCACAATGTTG |
| 584 | CGATACGCGCCGGTTTGTTAAATC | GATTTAACAAACCGGCGCGTATCG |
| 585 | GGCTATAAACGTGCGGACTGCTCC | GGAGCAGTCCGCACGTTTATAGCC |
| 586 | TGGGTAAATCACTATTGCGCGGTT | AACCGCGCAATAGTGATTTACCCA |
| 587 | GTCTTCATCGGCCCGCGCAAGCTA | TAGCTTGCGCGGGCCGATGAAGAC |
| 588 | GCGACACACCCTGTACTCTGATGC | GCATCAGAGTACAGGGTGTGTCGC |
| 589 | GTAGCAGGGTCCGCAAGACCAAGC | GCTTGGTCTTGCGGACCCTGCTAC |
| 590 | TCGCCAACGCAGGGTAACTGCCAT | ATGGCAGTTACCCTGCGTTGGCGA |
| 591 | ACTCCGAAGCTTCGAGCGGCACGA | TCGTGCCGCTCGAAGCTTCGGAGT |
| 592 | TCCCGCCCACTAGACTGACTCGTA | TACGAGTCAGTCTAGTGGGCGGGA |
| 593 | ACCTTCTGGGGTCGCTCACCAATA | TATTGGTGAGCGACCCCAGAAGGT |
| 594 | ATCATCCCACGGCAGAGTGAAGAG | CTCTTCACTCTGCCGTGGGATGAT |
| 595 | CGCTGGACTGGCCTATCCGAGTCG | CGACTCGGATAGGCCAGTCCAGCG |
| 596 | CGGTCTCAGCAACACTGTCGCAAA | TTTGCGACAGTGTTGCTGAGACCG |
| 597 | CGAACGTTCTCCGATGTAATGGCC | GGCCATTACATCGGAGAACGTTCG |
| 598 | ATACCGTGCGACAAGCCCCTCTGA | TCAGAGGGGCTTGTCGCACGGTAT |
| 599 | AGCTCATTCCCGAGACGGAACACC | GGTGTTCCGTCTCGGGAATGAGCT |
| 600 | TTTCATGCGGCCGTTGCAAATCAT | ATGATTTGCAACGGCCGCATGAAA |
| 601 | ACTCGAACGGACGTTCAATTCCCA | TGGGAATTGAACGTCCGTTCGAGT |
| 602 | CTGCATGGTGTGGGTGAGACTCCC | GGGAGTCTCACCCACACCATGCAG |
| 603 | CCGCGAGTGTGGATGGCGTGTTGA | TCAACACGCCATCCACACTCGCGG |
| 604 | AATGTGTCGGTCCTAAGCCGGGTG | CACCCGGCTTAGGACCGACACATT |
| 605 | TAAGACGAGCCTGCACAGCTTGCG | CGCAAGCTGTGCAGGCTCGTCTTA |
| 606 | GGCGTGGGAGGATAAGACGATGTC | GACATCGTCTTATCCTCCCACGCC |
| 607 | TGCTCCATGTTAGGAACGCACCAC | GTGGTGCGTTCCTAACATGGAGCA |
| 608 | CGGTGTTGGTCGGACTGACGACTG | CAGTCGTCAGTCCGACCAACACCG |
| 609 | CCGCGCGTATCTATCAGATCTGGG | CCCAGATCTGATAGATACGCGCGG |
| 610 | AAAGCATGCTCCACCTGGAGCGAG | CTCGCTCCAGGTGGAGCATGCTTT |
| 611 | ACTTGCATCGCTGGGTAGATCCGG | CCGGATCTACCCAGCGATGCAAGT |
| 612 | TGCTTACGCAGTGGATTGGTCAGA | TCTGACCAATCCACTGCGTAAGCA |
| 613 | ATGCAGATGAACAAATCGCCGAAT | ATTCGGCGATTTGTTCATCTGCAT |
| 614 | GCAATTCTGGGCCATGTATTCGTC | GACGAATACATGGCCCAGAATTGC |
| 615 | AGGGTTCCTTACGCGTCGACATGG | CCATGTCGACGCGTAAGGAACCCT |
| 616 | GTGGAGCTAATCGCGAGCCTCAGA | TCTGAGGCTCGCGATTAGCTCCAC |
| 617 | TCGTAGTCTCACCGGCAATGATCC | GGATCATTGCCGGTGAGACTACGA |
| 618 | TTATAGCAGTGCGCCAATGCTTCG | CGAAGCATTGGCGCACTGCTATAA |
| 619 | CGAACAGTGCTGTCCGTCGCTCAA | TTGAGCGACGGACAGCACTGTTCG |
| 620 | TCCGCGTGGACTGTTAGACGCTAT | ATAGCGTCTAACAGTCCACGCGGA |
| 621 | CATTAGCCCGCTGTCGGTAACTGT | ACAGTTACCGACAGCGGGCTAATG |
| 622 | GGAAAGAAACTCAGACGCGCAATG | CATTGCGCGTCTGAGTTTCTTTCC |
| 623 | CGACTCGCTGGACAGGAGAATCGT | ACGATTCTCCTGTCCAGCGAGTCG |
| 624 | CATGATCCTCTGTTTCACCCGCGG | CCGCGGGTGAAACAGAGGATCATG |
| 625 | GGCGTAGCGCTCTAAAAGCTTCGG | CCGAAGCTTTTAGAGCGCTACGCC |
| 626 | AGTGATGCCATCAGGCCCGTATAC | GTATACGGGCCTGATGGCATCACT |
| 627 | TATGGAAAGGGCAACAGCGCTATC | GATAGCGCTGTTGCCCTTTCCATA |
| 628 | CTGTGGTTGATGGAGGATCCACAC | GTGTGGATCCTCCATCAACCACAG |
| 629 | ACTCGCTGGAATTTGCGCTGACAC | GTGTCAGCGCAAATTCCAGCGAGT |
| 630 | CAGGCCCGAACCACGCGGTTACAG | CTGTAACCGCGTGGTTCGGGCCTG |
| 631 | GGCGCAATGGGCGCATAAATACTA | TAGTATTTATGCGCCCATTGCGCC |
| 632 | GGTCAATTCGCGCTACATGCCCTA | TAGGGCATGTAGCGCGAATTGACC |
| 633 | GATGGTGGACTGGAGCCCTTCCGC | GCGGAAGGGCTCCAGTCCACCATC |
| 634 | CCGCGCATAGCGCAATAGGGGAGA | TCTCCCCTATTGCGCTATGCGCGG |
| 635 | TCTTCTGGCTGTCCGGCACCCGAA | TTCGGGTGCCGGACAGCCAGAAGA |
| 636 | GCGTTCGCAATTCACGGGCCCTTA | TAAGGGCCCGTGAATTGCGAACGC |
| 637 | TCGTTTCGGCCTTGGAGAGTATCG | CGATACTCTCCAAGGCCGAAACGA |
| 638 | AGGTGCAAGTGCAAGGCGAGAGGC | GCCTCTCGCCTTGCACTTGCACCT |
| 639 | CGCCAGTTTCGATGGCTGACGTTT | AAACGTCAGCCATCGAAACTGGCG |
| 640 | GCTTTACCGCCGATCCCAGATATC | GATATCTGGGATCGGCGGTAAAGC |
| 641 | GTGCTTGACGAAGAGGCGAAATGT | ACATTTCGCCTCTTCGTCAAGCAC |
| 642 | CAGTCCGTGCGCTTCATGTCCTCA | TGAGGACATGAAGCGCACGGACTG |
| 643 | TACGCGTAAGAGCCTACCCTCGCG | CGCGAGGGTAGGCTCTTACGCGTA |
| 644 | GGCGAGTCTTGTGGGGACATGTGT | ACACATGTCCCCACAAGACTCGCC |
| 645 | CCAAAGCGAAGCGAGCGTGTCTAT | ATAGACACGCTCGCTTCGCTTTGG |
| 646 | GCCGTAGGTTGCTCTTCACCGAAC | GTTCGGTGAAGAGCAACCTACGGC |
| 647 | AAATCCGCGATGTGCCGTGAGGCT | AGCCTCACGGCACATCGCGGATTT |
| 648 | GGCTTCGCACCCGTACCAATTTAG | CTAAATTGGTACGGGTGCGAAGCC |
| 649 | TGTAGAGTCCCACGTAGCCGGCAT | ATGCCGGCTACGTGGGACTCTACA |
| 650 | CACTAGTCTGGGGCAAGGTGCATT | AATGCACCTTGCCCCAGACTAGTG |
| 651 | TGTACTCGGCAGGCGCAATAGATT | AATCTATTGCGCCTGCCGAGTACA |
| 652 | AACGGGTATCGGAAGCGTAAAAGC | GCTTTTACGCTTCCGATACCCGTT |
| 653 | CGGACTGCCCGTTTGCAAGTTGAG | CTCAACTTGCAAACGGGCAGTCCG |
| 654 | ATCGTTCAGCACTGGAGCCCGTAA | TTACGGGCTCCAGTGCTGAACGAT |
| 655 | ATGCATCGAACTAGTCGTGACGGC | GCCGTCACGACTAGTTCGATGCAT |
| 656 | TTCCAGGCATTAAGGAGAGGGAGC | GCTCCCTCTCCTTAATGCCTGGAA |
| 657 | GTGCGACATCTACTCCACGATCCC | GGGATCGTGGAGTAGATGTCGCAC |
| 658 | CTCATCGTCCTAACACGAGAGCCC | GGGCTCTCGTGTTAGGACGATGAG |
| 659 | AATGGCACTTCGGCGGTGATGCAA | TTGCATCACCGCCGAAGTGCCATT |
| 660 | CCGTGGGAGGGAATCCAACCGAGG | CCTCGGTTGGATTCCCTCCCACGG |
| 661 | AAATTCTCGTTGGTGACGGCTCAT | ATGAGCCGTCACCAACGAGAATTT |
| 662 | TTGCTCTTATCCTTGTCCTGGGCG | CGCCCAGGACAAGGATAAGAGCAA |
| 663 | TTAAGGATCAGGCGGAGCTTGCAG | CTGCAAGCTCCGCCTGATCCTTAA |
| 664 | CGCGACTAAGGTGCTGCAACTCGA | TCGAGTTGCAGCACCTTAGTCGCG |
| 665 | GCTCGATTTCACGGCCCGTTGTTC | GAACAACGGGCCGTGAAATCGAGC |
| 666 | AGCAGAGTGCGTTGCAGAGGCTAA | TTAGCCTCTGCAACGCACTCTGCT |
| 667 | TGGAGGTGAGGACGACGTGCACTA | TAGTGCACGTCGTCCTCACCTCCA |
| 668 | AACCGTTTAGGGTACATTCGCGGT | ACCGCGAATGTACCCTAAACGGTT |
| 669 | TATGATCGCTCGGCTCACAGTTTG | CAAACTGTGAGCCGAGCGATCATA |
| 670 | GACTTTTTGCGGAAACGTCATGGT | ACCATGACGTTTCCGCAAAAAGTC |
| 671 | TGTCGGTTATTCCACCTGCAAGGA | TCCTTGCAGGTGGAATAACCGACA |
| 672 | CTATGGTTTGCACTGCGCCGTCGA | TCGACGGCGCAGTGCAAACCATAG |
| 673 | AGCAGGGAAATTCAATCGTTCGCA | TGCGAACGATTGAATTTCCCTGCT |
| 674 | CCTAACCGAGCGCTTAGCATTTCC | GGAAATGCTAAGCGCTCGGTTAGG |
| 675 | CCCGACCCTAACTCGCATTGAATA | TATTCAATGCGAGTTAGGGTCGGG |
| 676 | TTGCTTAATGGTGACGCCACGGAT | ATCCGTGGCGTCACCATTAAGCAA |
| 677 | GATGCTCGCCGTGTTTAGTTCACG | CGTGAACTAAACACGGCGAGCATC |
| 678 | TCGGATGACGAGTTTCCATGACGG | CCGTCATGGAAACTCGTCATCCGA |
| 679 | ATGCGGTCTACTTTCTCGATCGGG | CCCGATCGAGAAAGTAGACCGCAT |
| 680 | TTGCGAGGCTAAGCACACGGTAAA | TTTACCGTGTGCTTAGCCTCGCAA |
| 681 | AACTTAATTACCGCCTCTGGCGCC | GGCGCCAGAGGCGGTAATTAAGTT |
| 682 | GTGACCGCGAACTTGTTCCGACAG | CTGTCGGAACAAGTTCGCGGTCAC |
| 683 | TGCGGATTACCGATTCGCTCTTAA | TTAAGAGCGAATCGGTAATCCGCA |
| 684 | TGATAGGGGGCCACGTTGATCAGA | TCTGATCAACGTGGCCCCCTATCA |
| 685 | TCGCTCCGTAGCGATTCATCGTAG | CTACGATGAATCGCTACGGAGCGA |
| 686 | TGTCAGCTGGTAGCCTCCGTTTGA | TCAAACGGAGGCTACCAGCTGACA |
| 687 | AGCGTCGCATGACGCTTACGGCAC | GTGCCGTAAGCGTCATGCGACGCT |
| 688 | TCACTCAGCGCTGTGACTGCCTGA | TCAGGCAGTCACAGCGCTGAGTGA |
| 689 | GTTTGCGCTATAGTGGGGGACCGT | ACGGTCCCCCACTATAGCGCAAAC |
| 690 | GTCGCATTCTGCACTGGCTTCGCC | GGCGAAGCCAGTGCAGAATGCGAC |
| 691 | TGATTAGGTGCGGTCCCGTAGTCC | GGACTACGGGACCGCACCTAATCA |
| 692 | AAGGGACCTTGGGTGACGGCGAGA | TCTCGCCGTCACCCAAGGTCCCTT |
| 693 | TCAAATGGCCACCGCGTGTCATTC | GAATGACACGCGGTGGCCATTTGA |
| 694 | CTCCGACGACCAATAAATAGCCGC | GCGGCTATTTATTGGTCGTCGGAG |
| 695 | GGCTATTCCCGTAGAGAGCGTCCA | TGGACGCTCTCTACGGGAATAGCC |
| 696 | TGGATAACCTCTCGGTCCATCCAC | GTGGATGGACCGAGAGGTTATCCA |
| 697 | GACCGCTGTACGGGAGTGTGCCTT | AAGGCACACTCCCGTACAGCGGTC |
| 698 | GCCACAGAGTTTTAGCAGGGACCC | GGGTCCCTGCTAAAACTCTGTGGC |
| 699 | CCCACGCTTTCCGACCACTGACCT | AGGTCAGTGGTCGGAAAGCGTGGG |
| 700 | CATTGACACAATGCGGGGACTGAT | ATCAGTCCCCGCATTGTGTCAATG |
| 701 | AGCCACTCGACAGGGTTCCAAAGC | GCTTTGGAACCCTGTCGAGTGGCT |
| 702 | CAGGATGAGCAAAGCGACTCTCCA | TGGAGAGTCGCTTTGCTCATCCTG |
| 703 | CAAGGTATGGTCTGGGGCCTAAGC | GCTTAGGCCCCAGACCATACCTTG |
| 704 | GGTGTTCGGCCTAAACTCTTTCGG | CCGAAAGAGTTTAGGCCGAACACC |
| 705 | TTTAGTCGGACCCTGTGGCAATTC | GAATTGCCACAGGGTCCGACTAAA |
| 706 | CACACGTTTCCGACCAGCCTGAAC | GTTCAGGCTGGTCGGAAACGTGTG |
| 707 | CTGGACGAACTGGCTTCCTCGTAC | GTACGAGGAAGCCAGTTCGTCCAG |
| 708 | TTCACAATCCGCCGAAAACTGACC | GGTCAGTTTTCGGCGGATTGTGAA |
| 709 | AACAGGATATCCGCGATCACGACA | TGTCGTGATCGCGGATATCCTGTT |
| 710 | TACGTCGGATCCATTGCGCCGAGT | ACTCGGCGCAATGGATCCGACGTA |
| 711 | CATGGATCTCTCGGTTTGATCGCC | GGCGATCAAACCGAGAGATCCATG |
| 712 | AGCCAGGCGCGTATATACGCTCGG | CCGAGCGTATATACGCGCCTGGCT |
| 713 | ATTTGGCACGTGTCGTGCCATGTT | AACATGGCACGACACGTGCCAAAT |
| 714 | CCGCGTTGCACCACTTTGAGGTGC | GCACCTCAAAGTGGTGCAACGCGG |
| 715 | TTGGACGTGACAAGCATGGCGCTC | GAGCGCCATGCTTGTCACGTCCAA |
| 716 | CTGAATCGCGCAAGTAAATGGGGG | CCCCCATTTACTTGCGCGATTCAG |
| 717 | GATAAGGTCCACCAGATTGCGCGC | GCGCGCAATCTGGTGGACCTTATC |
| 718 | CTAACAATTGCCAACCGGGACGGC | GCCGTCCCGGTTGGCAATTGTTAG |
| 719 | GGTAACCTGGGTGCTTGCAGGTTA | TAACCTGCAAGCACCCAGGTTACC |
| 720 | ATCGGAGCCACCATTCGCATTGGG | CCCAATGCGAATGGTGGCTCCGAT |
| 721 | GTGAACTGGCTTGCCCCAGGATTA | TAATCCTGGGGCAAGCCAGTTCAC |
| 722 | AGGCGATAGCATGGTCCCATATGA | TCATATGGGACCATGCTATCGCCT |
| 723 | AACGGTATCGTGGCTAATGCACGA | TCGTGCATTAGCCACGATACCGTT |
| 724 | AGTAGTGGTCCTCCAGATCGGCAA | TTGCCGATCTGGAGGACCACTACT |
| 725 | CCGTTGAATTGGACGGGAGGTTAG | CTAACCTCCCGTCCAATTCAACGG |
| 726 | GCATAAGTGCGGCATCGCGAAGGG | CCCTTCGCGATGCCGCACTTATGC |
| 727 | CGACAAGATGCAGCTGCTACATGC | GCATGTAGCAGCTGCATCTTGTCG |
| 728 | TCGCAGTGATTCCCGACCGATAAG | CTTATCGGTCGGGAATCACTGCGA |
| 729 | CAAGGCGAGTCCACTCGAGGGGAC | GTCCCCTCGAGTGGACTCGCCTTG |
| 730 | GCAACTTGCACGGCATAAGTGGCC | GGCCACTTATGCCGTGCAAGTTGC |
| 731 | TCCGAGCTTGACGTTCGCGACGTC | GACGTCGCGAACGTCAAGCTCGGA |
| 732 | AGCGCTGGGCTGTGCTGCCATCTC | GAGATGGCAGCACAGCCCAGCGCT |
| 733 | TTCATGTCGCTGAGTAACCCTCGC | GCGAGGGTTACTCAGCGACATGAA |
| 734 | CGAACCGCTAATGCCCATTGTCAG | CTGACAATGGGCATTAGCGGTTCG |
| 735 | CACGGAAGGTGGGACAAATCGCCG | CGGCGATTTGTCCCACCTTCCGTG |
| 736 | CACAGATGGAGACAAACGCGCCTT | AAGGCGCGTTTGTCTCCATCTGTG |
| 737 | TTTTCGCAACTCGCTCCATAACCC | GGGTTATGGAGCGAGTTGCGAAAA |
| 738 | ACGTTACGTTTCCGGCGCCTCTAA | TTAGAGGCGCCGGAAACGTAACGT |
| 739 | TATCGGATTGCGTGGGTTTCAATC | GATTGAAACCCACGCAATCCGATA |
| 740 | CTTCCACAATTGTCTGCGACGCAC | GTGCGTCGCAGACAATTGTGGAAG |
| 741 | TGCACAAAGGTATGGCTGTCCGGC | GCCGGACAGCCATACCTTTGTGCA |
| 742 | TCCGATGCCAGTCCCATCTTAAGA | TCTTAAGATGGGACTGGCATCGGA |
| 743 | CTGAAACCGTGCGAATCGAGGTGA | TCACCTCGATTCGCACGGTTTCAG |
| 744 | CGGTGTTCCGCGTGTCGAAAAAAT | ATTTTTTCGACACGCGGAACACCG |
| 745 | TCTAGCAGGCCTTTTGAATCGCCA | TGGCGATTCAAAAGGCCTGCTAGA |
| 746 | GAGTCACCTCTGAGACGGACGCCA | TGGCGTCCGTCTCAGAGGTGACTC |
| 747 | TCTTCTGTCATCCTGCAGCAGCAT | ATGCTGCTGCAGGATGACAGAAGA |
| 748 | GCGGATGAAACCTGAAAGGGGCCT | AGGCCCCTTTCAGGTTTCATCCGC |
| 749 | GGGGCCCCAAACTGGTATCAAGCC | GGCTTGATACCAGTTTGGGGCCCC |
| 750 | GCATTGGCTTCGGATTCTCCTACA | TGTAGGAGAATCCGAAGCCAATGC |
| 751 | AGGCGGCCCAACTGTGAGGTCTTG | CAAGACCTCACAGTTGGGCCGCCT |
| 752 | ACACCATGTGCTCCGCGCTGCAGT | ACTGCAGCGCGGAGCACATGGTGT |
| 753 | ACGATGAACATGAATCGGGAGTCG | CGACTCCCGATTCATGTTCATCGT |
| 754 | CTGCATCCCTGTAGCAGCGCTCCG | CGGAGCGCTGCTACAGGGATGCAG |
| 755 | GTGCCGTATTTCGACCTGTGCGTT | AACGCACAGGTCGAAATACGGCAC |
| 756 | GCAGTGCGCACTTCAGTTCAAAAG | CTTTTGAACTGAAGTGCGCACTGC |
| 757 | GCGATTTTAAGCGATGCCTTGACG | CGTCAAGGCATCGCTTAAAATCGC |
| 758 | TAGGTGACCTAGGCTTGCTTGCGG | CCGCAAGCAAGCCTAGGTCACCTA |
| 759 | CTGGATACCTTGCCTGTGCGGCGC | GCGCCGCACAGGCAAGGTATCCAG |
| 760 | CCCCTTACGGCTCGTCGTCTATGC | GCATAGACGACGAGCCGTAAGGGG |
| 761 | GCGCTTGCCCGATGCGATGCATTA | TAATGCATCGGATCGGGCAAGCGC |
| 762 | TTTCTGTAAGCGGCCTGGGGTTCA | TGAACCCCAGGCCGCTTACAGAAA |
| 763 | GGCTGAGGTGAGCGGTAAGGATGA | TCATCCTTACCGCTCACCTCAGCC |
| 764 | TCTTGGCCTCCCCGATCTAATTTG | CAAATTAGATCGGGGAGGCCAAGA |
| 765 | GGAGGTAACGCCGTGTACGTAGGA | TCCTACGTACACGGCGTTACCTCC |
| 766 | GTAATCCATTTGTGGCTGCGTCAA | TTGACGCAGCCACAAATGGATTAC |
| 767 | CAAACCCATTCCAGCAGACGCCTG | CAGGCGTCTGCTGGAATGGGTTTG |
| 768 | TAGGAGGAATTTGGCATGCGGGCG | CGCCCGCATGCCAAATTCCTCCTA |
| 769 | ATAGGTAGGATGTGCCCGGCGTTG | CAACGCCGGGCACATCCTACCTAT |
| 770 | GCAAGTGCTTAGCTCGTCAGCCTC | GAGGCTGACGAGCTAAGCACTTGC |
| 771 | CTGGCTGTGTCGCATCTCGTTAAC | GTTAACGAGATGCGACACAGCCAG |
| 772 | CTAACGTCGTCTCGCGCAATCACT | AGTGATTGCGCGAGACGACGTTAG |
| 773 | TTTTCATAAACGTTGTCCCCGAGC | GCTCGGGGACAACGTTTATGAAAA |
| 774 | AGCAGGAGGACGAACCTCCGCTCC | GGAGCGGAGGTTCGTCCTCCTGCT |
| 775 | TTCAAGCACCATCGTGCAATCCAA | TTGGATTGCACGATGGTGCTTGAA |
| 776 | AGCGTCGCCAGTGATCGCTAGTGG | CCACTAGCGATCACTGGCGACGCT |
| 777 | TACATTCCCTGCCTCCGTGGGCTT | AAGCCCACGGAGGCAGGGAATGTA |
| 778 | CGCTTCGCGTATTCAGTAGCGGTT | AACCGCTACTGAATACGCGAAGCG |
| 779 | TCGGACGCGTCGACACTCATTATA | TATAATGAGTGTCGACGCGTCCGA |
| 780 | TCTGAGCAGGCCAGCGCTCCAGCT | AGCTGGAGCGCTGGCCTGCTCAGA |
| 781 | TTGAATTGCCAAGCCCTGAAAGCC | GGCTTTCAGGGCTTGGCAATTCAA |
| 782 | AGTTTTCGCCTTGATGCGTCGGTG | CACCGACGCATCAAGGCGAAAACT |
| 783 | GTTTCATAGGCCACGCGTGCTAAA | TTTAGCACGCGTGGCCTATGAAAC |
| 784 | GGAGCGAAGACTTCGTCTGCCCAA | TTGGGCAGACGAAGTCTTCGCTCC |
| 785 | ATTGGCCGAGGGTGAATGCAGCCT | AGGCTGCATTCACCCTCGGCCAAT |
| 786 | TGATCCATCCGAATGCTTTTCCAT | ATGGAAAAGCATTCGGATGGATCA |
| 787 | GCACACAGTTGTCTTGGCCCATGA | TCATGGGCCAAGACAACTGTGTGC |
| 788 | CTGGCGGGCAGTGGAAAAAACAAC | GTTGTTTTTTCCACTGCCCGCCAG |
| 789 | ATCTCCATGCGTAAGACTGCTCCG | CGGAGCAGTCTTACGCATGGAGAT |
| 790 | TCTCCTCTCGTCGCAGTTCGTGGA | TCCACGAACTGCGACGAGAGGAGA |
| 791 | TAGCGTATTCACTCTTGCCGAGCA | TGCTCGGCAAGAGTGAATACGCTA |
| 792 | CAATCAAAAGCCACGGCGCGATGG | CCATCGCGCCGTGGCTTTTGATTG |
| 793 | AGCGTCACGGAATTCAGCAGATCT | AGATCTGCTGAATTCCGTGACGCT |
| 794 | GACTCCCTGTTAATGCGCCCAAGG | CCTTGGGCGCATTAACAGGGAGTC |
| 795 | TAGGCACTGCCGGTTCAGATTCAA | TTGAATCTGAACCGGCAGTGCCTA |
| 796 | AACAGGGTGATAACGGTGGCCAAT | ATTGGCCACCGTTATCACCCTGTT |
| 797 | CGTGCGTACCATGTGTAAGTGCGT | ACGCACTTACACATGGTACGCACG |
| 798 | GACCAATTCTACTTCGGCAGCCCA | TGGGCTGCCGAAGTAGAATTGGTC |
| 799 | ATCGGACCGATTTGCTTTTGGCTG | CAGCCAAAAGCAAATCGGTCCGAT |
| 800 | TCCGCCGAAGCACACGCTTATTCG | CGAATAAGCGTGTGCTTCGGCGGA |
| 801 | AACGGTACGCATTGTGAGCAGTGT | ACACTGCTCACAATGCGTACCGTT |
| 802 | TGGCGACTACTGTTCCCCTGAATC | GATTCAGGGGAACAGTAGTCGCCA |
| 803 | CAGAGGGGACAGCCGTATGCCTTA | TAAGGCATACGGCTGTCCCCTCTG |
| 804 | CGGTGGTTTTATCGGAATCTGCGA | TCGCAGATTCCGATAAAACCACCG |
| 805 | TTGGCCTCCGACCTCACGACATAT | ATATGTCGTGAGGTCGGAGGCCAA |
| 806 | CGTTTCGCTAGCATCTGGCGCCGA | TCGGCGCCAGATGCTAGCGAAACG |
| 807 | ACTAAGCGGTGGAGCCGGTGGATG | CATCCACCGGCTCCACCGCTTAGT |
| 808 | ATATTGGCTGCGTTTACGGGCCGC | GCGGCCCGTAAACGCAGCCAATAT |
| 809 | CCGCTATGGTGGCAATCCCGATAC | GTATCGGGATTGCCACCATAGCGG |
| 810 | GTTGCATGTGGCTCAGGCGGCATA | TATGCCGCCTGAGCCACATGCAAC |
| 811 | ATTCTGGGGAGTGACCCAGGGCTT | AAGCCCTGGGTCACTCCCCAGAAT |
| 812 | CTCTCCAAGGAGACGAGCCAATGT | ACATTGGCTCGTCTCCTTGGAGAG |
| 813 | GAAAGGACGGGATTTGGGGGCTAA | TTAGCCCCCAAATCCCGTCCTTTC |
| 814 | TATGTAGTACCTTGGCTCGCGCCA | TGGCGCGAGCCAAGGTACTACATA |
| 815 | TCCCTTTCGATGAGCGGCTGTACT | AGTACAGCCGCTCATCGAAAGGGA |
| 816 | TAGATCGGGCAGAGCCCGTATCTT | AAGATACGGGCTCTGCCCGATCTA |
| 817 | GGAATGCTTTAGGCTGCCGAGCTG | CAGCTCGGCAGCCTAAAGCATTCC |
| 818 | ATGGTAGCAACATTCAACGCCAGG | CCTGGCGTTGAATGTTGCTACCAT |
| 819 | CTATGAAACGTGTGGCCCAGCAAC | GTTGCTGGGCCACACGTTTCATAG |
| 820 | ATGTTGCTAGTGCCTTTCGGGCCT | AGGCCCGAAAGGCACTAGCAACAT |
| 821 | CCAATGTGCGCAGACTCAGTCATT | AATGACTGAGTCTGCGCACATTGG |
| 822 | GATAGTGCTCGCAAACGGGCCTTC | GAAGGCCCGTTTGCGAGCACTATC |
| 823 | GCACCCTGTTGCCTCATTGAGCGT | ACGCTCAATGAGGCAACAGGGTGC |
| 824 | GGCGTGAATAGAGTGACCAGGCGG | CCGCCTGGTCACTCTATTCACGCC |
| 825 | ACGTGCCAGCTGCGGGCACTTTAT | ATAAAGTGCCCGCAGCTGGCACGT |
| 826 | AGTGGAATAGTCGCGTCGTGCCGC | GCGGCACGACGCGACTATTCCACT |
| 827 | ACTCGCCTATTACCGCTGGATTGG | CCAATCCAGCGGTAATAGGCGAGT |
| 828 | GAGACCGGATTGAGATGATCCCGT | ACGGGATCATCTCAATCCGGTCTC |
| 829 | CTGGCAGTTTACCACCGAACCAGT | ACTGGTTCGGTGGTAAACTGCCAG |
| 830 | TTACATTGCCGATTTCGCATGTGA | TCACATGCGAAATCGGCAATGTAA |
| 831 | TAAAACTGAAGGGTCGCCTCAGCA | TGCTGAGGCGACCCTTCAGTTTTA |
| 832 | GGCTTCGCATGCCTTTGCAACATT | AATGTTGCAAAGGCATGCGAAGCC |
| 833 | AAGACCGAAGGTCTCTCTGAGGGC | GCCCTCAGAGAGACCTTCGGTCTT |
| 834 | GCCTATGGCTCCAGCTCAGCAGTA | TACTGCTGAGCTGGAGCCATAGGC |
| 835 | CGTATCATAGCGTTCGGTGGACAA | TTGTCCACCGAACGCTATGATACG |
| 836 | CATGCGCTCGCACTCTGCCTGTCT | AGACAGGCAGAGTGCGAGCGCATG |
| 837 | TGGGCAATTCGGAAACGTCGGTCT | AGACCGACGTTTCCGAATTGCCCA |
| 838 | TTGCGGAGATGCGACGGTACATTG | CAATGTACCGTCGCATCTCCGCAA |
| 839 | ACTTTCGCACGTCGATCTGGACTG | CAGTCCAGATCGACGTGCGAAAGT |
| 840 | CTAACTGCCGCGGCAAACTGATTA | TAATCAGTTTGCCGCGGCAGTTAG |
| 841 | GGCCGCGGATTTTATTCCTTGGAT | ATCCAAGGAATAAAATCCGCGGCC |
| 842 | GAATTTGGAACGGTGTTCCGATGA | TCATCGGAACACCGTTCCAAATTC |
| 843 | GTCCATCCATCTACGGCATCAGGA | TCCTGATGCCGTAGATGGATGGAC |
| 844 | TAAACGACCTGGCACATGTGCGTA | TACGCACATGTGCCAGGTCGTTTA |
| 845 | CACCATCCAAGAGCCAATCCTAGG | CCTAGGATTGGCTCTTGGATGGTG |
| 846 | ACTCATATACGATCAGTCCGCCGC | GCGGCGGACTGATCGTATATGAGT |
| 847 | GTGCCAACCGACGATCAACCGAAC | GTTCGGTTGATCGTCGGTTGGCAC |
| 848 | TGGGGTTCGTACAGGTCGGTTCAT | ATGAACCGACCTGTACGAACCCCA |
| 849 | AACAGTAGAGGCGAGGCCTGCGGG | CCCGCAGGCCTCGCCTCTACTGTT |
| 850 | TGCATCGAATCCGAGATGGATCTT | AAGATCCATCTCGGATTCGATGCA |
| 851 | GCGTCACGTTATGTCCGCTCTGTC | GACAGAGCGGACATAACGTGACGC |
| 852 | GGGACATGCGTAGCGCAATATCAC | GTGATATTGCGCTACGCATGTCCC |
| 853 | CACACGTCACACCATCCAAAGTGG | CCACTTTGGATGGTGTGACGTGTG |
| 854 | ATGCTCAGGTGCTAAATACGGCCA | TGGCCGTATTTAGCACCTGAGCAT |
| 855 | AAAAATGTTTAGCGCGCTGACTGG | CCAGTCAGCGCGCTAAACATTTTT |
| 856 | ATAGTCCGTTTCCGTTCCCAACGA | TCGTTGGGAACGGAAACGGACTAT |
| 857 | TCGATCTTCTGGGTTGCAGACCAG | CTGGTCTGCAACCCAGAAGATCGA |
| 858 | GTCGGCGCAGCCGATCCTCATGTC | GACATGAGGATCGGCTGCGCCGAC |
| 859 | GTTGCGGGGTGTCGAAAAGGATCT | AGATCCTTTTCGACACCCCGCAAC |
| 860 | ATCTCTTCCTCGGGTGGATGCCAG | CTGGCATCCACCCGAGGAAGAGAT |
| 861 | TGATGTGCGTTTCAGCTTTTCGCG | CGCGAAAAGCTGAAACGCACATCA |
| 862 | GTTAAGGGGTGAGAACATCCGGCC | GGCCGGATGTTCTCACCCCTTAAC |
| 863 | AAGTCGTCTCCCTGCGTCTCGTCC | GGACGAGACGCAGGGAGACGACTT |
| 864 | CCGACCTAATAAGGCGCAACAATG | CATTGTTGCGCCTTATTAGGTCGG |
| 865 | CATCATTGGCACCGTACCAATGCC | GGCATTGGTACGGTGCCAATGATG |
| 866 | TGGAGAAAGGGAAGTGCAGCAACG | CGTTGCTGCACTTCCCTTTCTCCA |
| 867 | TGGTACTCCTTGTCATGCCTGCCA | TGGCAGGCATGACAAGGAGTACCA |
| 868 | GGCACAGGTTCTCTTGCAGCGCGG | CCGCGCTGCAAGAGAACCTGTGCC |
| 869 | GAATCTGGGCATTGCTACGAGACC | GGTCTCGTAGCAATGCCCAGATTC |
| 870 | CGAAATGGGAGCGTCCACTACCAC | GTGGTAGTGGACGCTCCCATTTCG |
| 871 | ACATATGAGCTCGCGTGCTTGCAT | ATGCAAGCACGCGAGCTCATATGT |
| 872 | TCGAGCACGGTCACTGATAAAGCC | GGCTTTATCAGTGACCGTGCTCGA |
| 873 | GAGGGTCCCTGCTCAGAGTTGGTT | AACCAACTCTGAGCAGGGACCCTC |
| 874 | AAATGCGATCGCCCCTTATGGAAT | ATTCCATAAGGGGCGATCGCATTT |
| 875 | CTACCCGAATGGATTGCGGATGGC | GCCATCCGCAATCCATTCGGGTAG |
| 876 | AGGGACTGGCAGGTCTCTGCGCGT | ACGCGCAGAGACCTGCCAGTCCCT |
| 877 | TAACGATCCATTCCACGAATGCAG | CTGCATTCGTGGAATGGATCGTTA |
| 878 | GGCCGCACGTACGATTACGCCTTG | CAAGGCGTAATCGTACGTGCGGCC |
| 879 | TGGGGAATGCATCAGTTGTTGGCT | AGCCAACAACTGATGCATTCCCCA |
| 880 | TATCTGGGAGTAGCAGGCAGGGCC | GGCCCTGCCTGCTACTCCCAGATA |
| 881 | CCGAAGGTTTCACGCTCAGGTCGC | GCGACCTGAGCGTGAAACCTTCGG |
| 882 | GAACCCAGCTGGGACATCCTTCAG | CTGAAGGATGTCCCAGCTGGGTTC |
| 883 | TGCATGCGAGCAAATAACCCGGAC | GTCCGGGTTATTTGCTCGCATGCA |
| 884 | AATTGTCCGCCAAACGCTTTTCAG | CTGAAAAGCGTTTGGCGGACAATT |
| 885 | GTCGGCTTCGAGCGATCGAGTGTG | CACACTCGATCGCTCGAAGCCGAC |
| 886 | TCGCGTGCTCTACGTAGCCCATGA | TCATGGGCTACGTAGAGCACGCGA |
| 887 | GGCTTCCGCGATAACGTAATTCGC | GCGAATTACGTTATCGCGGAAGCC |
| 888 | TGTAGCCGACTAGGGCCGAAGCCC | GGGCTTCGGCCCTAGTCGGCTACA |
| 889 | AAGCGAACGCCCTGGCTGAATATT | AATATTCAGCCAGGGCGTTCGCTT |
| 890 | TGTCACGCGACGTGCTGCAGATTT | AAATCTGCAGCACGTCGCGTGACA |
| 891 | CCGTGTCCGTGTTGTCGACAGGCG | CGCCTGTCGACAACACGGACACGG |
| 892 | CCCCACACGTTGCGCCTATATGTG | CACATATAGGCGCAACGTGTGGGG |
| 893 | GGCGGGCACAACTCAACACAGATG | CATCTGTGTTGAGTTGTGCCCGCC |
| 894 | CGACTGCGGGATCACCGGTGATTA | TAATCACCGGTGATCCCGCAGTCG |
| 895 | TCGGGACATGACCGGTACGGAGTC | GACTCCGTACCGGTCATGTCCCGA |
| 896 | TACCTCGAGTGGCCGTTGATCGGG | CCCGATCAACGGCCACTCGAGGTA |
| 897 | TAATTCATGGGGCTAGCCGAACCA | TGGTTCGGCTAGCCCCATGAATTA |
| 898 | ACACTCTAAGCCGATTCCGTTCGA | TCGAACGGAATCGGCTTAGAGTGT |
| 899 | GTGGGCGTGAGTGACACGCACAAA | TTTGTGCGTGTCACTCACGCCCAC |
| 900 | ACGACTCCTCGGGCAAAGTACGTA | TACGTACTTTGCCCGAGGAGTCGT |
| 901 | TGTGGTCATGGCGCTACTGTTTTC | GAAAACAGTAGCGCCATGACCACA |
| 902 | CTTTCGCTAGCCAGAGCGGGTTCC | GGAACCCGCTCTGGCTAGCGAAAG |
| 903 | ACAGGGCGTGTTAGCGTGTGACAA | TTGTCACACGCTAACACGCCCTGT |
| 904 | GGTACTTCCGGCGTATCGGGCCAC | GTGGCCCGATACGCCGGAAGTACC |
| 905 | GTGGGTTTTGTTCACCCTTCTGGG | CCCAGAAGGGTGAACAAAACCCAC |
| 906 | ACGCAATTCCGCATTACTTACCCG | CGGGTAAGTAATGCGGAATTGCGT |
| 907 | CGCCTCGACTGCGGTCAAGCACAA | TTGTGCTTGACCGCAGTCGAGGCG |
| 908 | GTGAAATGGATCCAGAGAGGGCCA | TGGCCCTCTCTGGATCCATTTCAC |
| 909 | TATAAACGCTGCAGGGCTCCGTTA | TAACGGAGCCCTGCAGCGTTTATA |
| 910 | GTTATTCAGGCGGCTTGTAACGGG | CCCGTTACAAGCCGCCTGAATAAC |
| 911 | GGGTTCTAGCGTGCGCGTTCAGTT | AACTGAACGCGCACGCTAGAACCC |
| 912 | TTGGGCTCGAGCGGTACACCACTA | TAGTGGTGTACCGCTCGAGCCCAA |
| 913 | CCGTCTTCAGGACAACGGTATGCG | CGCATACCGTTGTCCTGAAGACGG |
| 914 | GGACCCTTTGACAGATTGCGGCAC | GTGCCGCAATCTGTCAAAGGGTCC |
| 915 | TAAATTTTATCGCCAGGCGGCGCT | AGCGCCGCCTGGCGATAAAATTTA |
| 916 | GCCGAACGCAAGATCGCTTGAACT | AGTTCAAGCGATCTTGCGTTCGGC |
| 917 | TAGGCCATTGGTGCCCTAAGACGG | CCGTCTTAGGGCACCAATGGCCTA |
| 918 | CAAACCACAGCTTACAGGCTGCGT | ACGCAGCCTGTAAGCTGTGGTTTG |
| 919 | TAAACGGAGACTGGCACGGTAGCA | TGCTACCGTGCCAGTCTCCGTTTA |
| 920 | TAGCGCGCATCACACTTGGAATCG | CGATTCCAAGTGTGATGCGCGCTA |
| 921 | TGCTGACACAAACGAGCCGTTTCG | CGAAACGGCTCGTTTGTGTCAGCA |
| 922 | CGCTTAACGGCATTGACTGTCCAC | GTGGACAGTCAATGCCGTYAAGCG |
| 923 | TTCCACGGCCGTGTATTACGGATA | TATCCGTAATACACGGCCGTGGAA |
| 924 | TTTATGCCGTTGCCGAGGAAGACT | AGTCTTCCTCGGCAACGGCATAAA |
| 925 | AGTGCCGAGATAGGGGACTGGGCG | CGCCCAGTCCCCTATCTCGGCACT |
| 926 | CTAGTCTCCACGCCCTCGGGACGA | TCGTCCCGAGGGCGTGGAGACTAG |
| 927 | CCGCCATTCGGAAGATGGATGATG | CATCATCCATCTTCCGAATGGCGG |
| 928 | TGACGGTGAAAGTCGATTGCGAAG | CTTCGCAATCGACTTTCACCGTCA |
| 929 | ATATGCGTCACCACCCGGTTCCGA | TCGGAACCGGGTGGTGACGCATAT |
| 930 | CCATCAGTGAAGGGGTTGCTGCCA | TGGCAGCAACCCCTTCACTGATGG |
| 931 | CATATGTGCTTGGCTTGCGATGAC | GTCATCGCAAGCCAAGCACATATG |
| 932 | TCTGCTTTGGAAGCCTGAACTGCT | AGCAGTTCAGGCTTCCAAAGCAGA |
| 933 | CGATTTGGTCAAGAAGGCGGAAAT | ATTTCCGCCTTCTTGACCAAATCG |
| 934 | ATCAGAGGCCTTCCCGCCTCGTTA | TAACGAGGCGGGAAGGCCTCTGAT |
| 935 | ATTGTTGTCGTTGCCACATCGCAG | CTGCGATGTGGCAACGACAACAAT |
| 936 | TGAAATGTGTCTGGACGCGAGTCT | AGACTCGCGTCCAGACACATTTCA |
| 937 | GCGGGCGATGCTCCTTAAAGGGTA | TACCCTTTAAGGAGCATCGCCCGC |
| 938 | CCGCAATCTCCATGCGTCGACCGT | ACGGTCGACGCATGGAGATTGCGG |
| 939 | TGCCGCGTAATCACCTGGAACTTG | CAAGTTCCAGGTGATTACGCGGCA |
| 940 | TTCCAGTAGCCAGCGGTAGTGTGA | TCACACTACCGCTGGCTACTGGAA |
| 941 | CTGAATTCCGCCTATTGTTCGGCA | TGCCGAACAATAGGCGGAATTCAG |
| 942 | GCTTGAACCTCGAGGCGATGTTCT | AGAACATCGCCTCGAGGTTCAAGC |
| 943 | CAAGCGTGGAAGTACGACCCGCCA | TGGCGGGTCGTACTTCCACGCTTG |
| 944 | GTGTGCACTGGATCCGAGCCCTAG | CTAGGGCTCGGATCCAGTGCACAC |
| 945 | TCCCTGGGCTAGCATTGCGAGGTT | AACCTCGCAATGCTAGCCCAGGGA |
| 946 | AGAACCAAAGACGCTTGTTTGCCG | CGGCAAACAAGCGTCTTTGGTTCT |
| 947 | CGTCACATGCAAACGTTCCCTCCC | GGGAGGGAACGTTTGCATGTGACG |
| 948 | TGACCGCATGTGTATTGAGTCGCT | AGCGACTCAATACACATGCGGTCA |
| 949 | GCGGGCCCAATGAGTATCCGTCAT | ATGACGGATACTCATTGGGCCCGC |
| 950 | TAGTGACTGTGAACGCCCCTGGTT | AACCAGGGGCGTTCACAGTCACTA |
| 951 | GGCACCGTCTGCCGCGCGTATATC | GATATACGCGCGGCAGACGGTGCC |
| 952 | TCGATGCAGTCTTTTTCCCGTCAA | TTGACGGGAAAAAGACTGCATCGA |
| 953 | ACCCCGTGGGGTTTCGCCATTTTT | AAAAATGGCGAAACCCCACGGGGT |
| 954 | CTACACGCGCAGTTGTGACTTGTG | GACAAGTCACAACTGCGCGTGTAG |
| 955 | CGCAGCGACCTCATCTCTGGAGCC | GGCTCCAGAGATGAGGTCGCTGCG |
| 956 | CGACCCAGCACTCCTAAAATCGGT | ACCGATTTTAGGAGTGCTGGGTCG |
| 957 | ACGCGCCGCTCATCACTACAATCT | AGATTGTAGTGATGAGCGGCGCGT |
| 958 | CGCAACTTCCTGTGGCAAAGCCAG | CTGGCTTTGCCACAGGAAGTTGCG |
| 959 | TCGTTGGGCACATAAGGCAACTGA | TCAGTTGCCTTATGTGCCCAACGA |
| 960 | CCGCTTGTAATTGCCATTCTCCGT | ACGGAGAATGGCAATTACAAGCGG |
| 961 | GTAACCAGGGAGTCCTGGGCTGTG | CACAGCCCAGGACTCCCTGGTTAC |
| 962 | AGCGCAAGATCTGGGGGCAGTCAC | GTGACTGCCCCCAGATCTTGCGCT |
| 963 | GCGTACATCTGCTCATCAGCATGG | CCATGCTGATGAGCAGATGTACGC |
| 964 | CCTCTGTGGCAGGAAAGAAACCGT | ACGGTTTCTTTCCTGCCACAGAGG |
| 965 | CCTATGCAATGGACCTGCATCGGA | TCCGATGCAGGTCCATTGCATAGG |
| 966 | CTCGGTGGATGGCGAATAAGGATA | TATCCTTATTCGCCATCCACCGAG |
| 967 | CCTCACTCGTGATGGCGTGACGCA | TGCGTCACGCCATCACGAGTGAGG |
| 968 | TACGCTCACAGAACGCCATACGCC | GGCGTATGGCGTTCTGTGAGCGTA |
| 969 | CCGGAGAAGTTACGCGGATCGGAC | GTCCGATCCGCGTAACTTCTCCGG |
| 970 | GCGCCCTCACTGCATTTTTGGTAT | ATACCAAAAATGCAGTGAGGGCGC |
| 971 | ACTTTCAGCACGCGAACAGCGCAA | TTGCGCTGTTCGCGTGCTGAAAGT |
| 972 | CTAAACGCCCTTGATGCATGAGCA | TGCTCATGCATCAAGGGCGTTTAG |
| 973 | GCTTGCCTTTTACGATCGTCGCTA | TAGCGACGATCGTAAAAGGCAAGC |
| 974 | CAGACATCGTACGCACTCGGCATC | GATGCCGAGTGCGTACGATGTCTG |
| 975 | TAGCCGCGCGGCTCCTATGCTCTT | AAGAGCATAGGAGCCGCGCGGCTA |
| 976 | GATGCCCTTTTGGTCCCCATGCCA | TGGCATGGGGACCAAAAGGGCATC |
| 977 | TGAGCTGCCTTGCCACGATGCCTC | GAGGCATCGTGGCAAGGCAGCTCA |
| 978 | CCGCCGTATACGTGCCATAGTTTG | CAAACTATGGCACGTATACGGCGG |
| 979 | TAGTGCTCTCCGCGCTCATCCAAC | GTTGGATGAGCGCGGAGAGCACTA |
| 980 | CCCTAGATAAGTTGGGGTGGGACG | CGTCCCACCCCAACTTATCTAGGG |
| 981 | TGAAGGGCCACCTGATATGGTTTC | GAAACCATATCAGGTGGCCCTTCA |
| 982 | GCCGCCTCCGACTGGTTAACCCGA | TCGGGTTAACCAGTCGGAGGCGGC |
| 983 | CGCACGGCTACTAACAGCGGATCA | TGATCCGCTGTTAGTAGCCGTGCG |
| 984 | CCGGACCAATTCCAACGAGCATCG | CGATGCTCGTTGGAATTGGTCCGG |
| 985 | CATTGAGGTCCACCGTTCACATCC | GGATGTGAACGGTGGACCTCAATG |
| 986 | AGGACGCAGCATGTCCCAGCCGAG | CTCGGCTGGGACATGCTGCGTCCT |
| 987 | TAATCGCGGGCCATACTACCAACG | CGTTGGTAGTATGGCCCGCGATTA |
| 988 | CGCAAATTTCTCCGGTCGGCAAGC | GCTTGCCGACCGGAGAAATTTGCG |
| 989 | GTGGCTCGACTAATGCCTTGCGTG | CACGCAAGGCATTAGTCGAGCCAC |
| 990 | TGTGGGCGTGTTCCGGCTCACTGT | ACAGTGAGCCGGAACACGCCCACA |
| 991 | GTTCTTCCTTTTCTGCGGTGGGAA | TTCCCACCGCAGAAAAGGAAGAAC |
| 992 | ACCTCGAGTCAGATTGTGCGCCTT | AAGGCGCACAATCTGACTCGAGGT |
| 993 | CAAGTGGACAGACGGTTTGTTCCG | CGGAACAAACCGTCTGTCCACTTG |
| 994 | TCCAGTTGAGTCGCGCCGACGAGG | CCTCGTCGGCGCGACTCAACTGGA |
| 995 | CGCAACAGGTCAGCCCTTATTTGC | GCAAATAAGGGCTGACCTGTTGCG |
| 996 | GCCGTGACTCCTGCAATGTCGGTA | TACCGACATTGCAGGAGTCACGGC |
| 997 | ATCAGCGCAAGCTGGTCTGAAACA | TGTTTCAGACCAGCTTGCGCTGAT |
| 998 | CCCTGGCCAGAACGAGAGGCCATG | CATGGCCTCTCGTTCTGGCCAGGG |
| 999 | ACGATCAAGGACTCGTCAGGGTTG | CAACCCTGACGAGTCCTTGATCGT |
| 1000 | TTCATGGCACCAAGACCACCGTTA | TAACGGTGGTCTTGGTGCCATGAA |
| 1001 | ACAGCAAGGAGATGGATTGCGACG | CGTCGCAATCCATCTCCTTGCTGT |
| 1002 | CGTAAATATCTGCGGCGGTGTGAA | TTCACACCGCCGCAGATATTTACG |
| 1003 | GGAAACACGTGTTCGTCTGTTGGC | GCCAACAGACGAACACGTGTTTCC |
| 1004 | CGATGTTAGGATTCGGATAGGCCA | TGGCCTATCCGAATCCTAACATCG |
| 1005 | ATCGGACAAGGACAAGTGGATGGT | ACCATCCACTTGTCCTTGTCCGAT |
| 1006 | GCCCGGAGGACAAAGTTCGAGTTA | TAACTCGAACTTTGTCCTCCGGGC |
| 1007 | AAATCCGACAAATGGGCACATGGA | TCCATGTGCCCATTTGTCGGATTT |
| 1008 | CAGTTAGGGGATGCGGATGAGTGA | TCACTCATCCGCATCCCCTAACTG |
| 1009 | CGGCAGGTGGAGATTCCGACATTG | CAATGTCGGAATCTCCACCTGCCG |
| 1010 | TAGGGCAGCCAGGTTCACTCATCT | AGATGAGTGAACCTGGCTGCCCTA |
| 1011 | GCACCGTATTAGCAGTAGGCACGC | GCGTGCCTACTGCTAATACGGTGC |
| 1012 | ACGCATTACAGGTGTGCGAAGGGA | TCCCTTCGCACACCTGTAATGCGT |
| 1013 | CGTGACTGCACGTGTTCCACAGGG | CCCTGTGGAACACGTGCAGTCACG |
| 1014 | GCTGAACTACCGCCTAAAATCGCG | CGCGATTTTAGGCGGTAGTTCAGC |
| 1015 | AGCACGCCAGGGAGGATCGAGTTA | TAACTCGATCCTCCCTGGCGTGCT |
| 1016 | ATGAGGGCAAGGAATGGGTCATGC | GCATGACCCATTCCTTGCCCTCAT |
| 1017 | GGGTCTCTCGTAATCAAAGGCCGA | TCGGCCTTTGATTACGAGAGACCC |
| 1018 | TATCTTGCGCAACGCCTCCATTTA | TAAATGGAGGCGTTGCGCAAGATA |
| 1019 | GGTTACACCTACGGAATCCAGCGG | CCGCTGGATTCCGTAGGTGTAACC |
| 1020 | ACACCGAGTTGGTCCGGTCAATAG | CTATTGACCGGACCAACTCGGTGT |
| 1021 | TCCCAGATTAAACGCTAGCCACCG | CGGTGGCTAGCGTTTAATCTGGGA |
| 1022 | TTGGTGAAACTGGCCCGTCGGAAG | CTTCCGACGGGCCAGTTTCACCAA |
| 1023 | CCAGGGGAGTTGACAATGAGGCTG | CAGCCTCATTGTCAACTCCCCTGG |
| 1024 | TCTGCGTTATTGGACCGTTTGTCG | CGACAAACGGTCCAATAACGCAGA |
| 1025 | TATGGGATGCTAAACCGGCGTACA | TGTACGCCGGTTTAGCATCCCATA |
| 1026 | CACAGACGTCTGTCGGGCTTGTGT | ACACAAGCCCGACAGACGTCTGTG |
| 1027 | AGAATGCCGTTCGCCTACTCCCGT | ACGGGAGTAGGCGAACGGCATTCT |
| 1028 | CGACGGATAATGCAGGCCTCATGA | TCATGAGGCCTGCATTATCCGTCG |
| 1029 | ACCCTCTAAAGCAATAGGTCGGCG | CGCCGACCTATTGCTTTAGAGGGT |
| 1030 | CACTCACGGCAGAAGCCTGCTTGT | ACAAGCAGGCTTCTGCCGTGAGTG |
| 1031 | ATCAGCCCACATATTCTCGGCCGT | ACGGCCGAGAATATGTGGGCTGAT |
| 1032 | CAAATCTGGGGTCGTCCTAAACGC | GCGTTTAGGACGACCCCAGATTTG |
| 1033 | TGTCGCCCATGGCAGGTTAAATAC | GTATTTAACCTGCCATGGGCGACA |
| 1034 | GGGGGCCCATCAATTCATTATCGA | TCGATAATGAATTGATGGGCCCCC |
| 1035 | GTCGAGCAGCTTTAGTATCGCGGG | CCCGCGATACTAAAGCTGCTCGAC |
| 1036 | CCGCTAAGCACCGAAGGCTCACAA | TTGTGAGCCTTCGGTGCTTAGCGG |
| 1037 | TAGAATTAGCGAACGGTGATCCCG | CGGGATCACCGTTCGCTAATTCTA |
| 1038 | CACATGACATTTGGCAAAGGTCCA | TGGACCTTTGCCAAATGTCATGTG |
| 1039 | TCAACGCACTGGCGATGACTAGAT | ATCTAGTCATCGCCAGTGCGTTGA |
| 1040 | CGGGAAATGTCTTTAGCCGTCGAA | TTCGACGGCTAAAGACATTTCCCG |
| 1041 | ATCAGAGCAAATCTGCAGCGGGGA | TCCCCGCTGCAGATTTGCTCTGAT |
| 1042 | GGCCTGTTTCTGTCCAACTGGGCT | AGCCCAGTTGGACAGAAACAGGCC |
| 1043 | ATTTCACCTCGCTGATCGCTTCCG | CGGAAGCGATCAGCGAGGTGAAAT |
| 1044 | AGTGACGCCGAGTCGCGAGGGTTA | TAACCCTCGCGACTCGGCGTCACT |
| 1045 | AGTTGTCTCATCCTGTCCGGGACC | GGTCCCGGACAGGATGAGACAACT |
| 1046 | CTTCTTTGTGCACACTTGCCAGGG | CCCTGGCAAGTGTGCACAAAGAAG |
| 1047 | CACCTCATCGGAGCATAGCAACCC | GGGTTGCTATGCTCCGATGAGGTG |
| 1048 | ATGCGATCCATGACAAGGGTTGCT | AGCAACCCTTGTCATGGATCGCAT |
| 1049 | CCCGTGGAGATGATGTGCGGCTTA | TAAGCCGCACATCATCTCCACGGG |
| 1050 | CCCAATAGACGCCACAGCCAGTGA | TCACTGGCTGTGGCGTCTATTGGG |
| 1051 | AACGACCACGACCCTCGCCGAGTA | TACTCGGCGAGGGTCGTGGTCGTT |
| 1052 | GGTGCTTTGTCTGAGGCGAGTGAA | TTCACTCGCCTCAGACAAAGCACC |
| 1053 | CTGTCGGCGCTGCTCTCCGAATTT | AAATTCGGAGAGCAGCGCCGACAG |
| 1054 | CTCGCCGGAGTGTTGTAAGCATTG | CAATGCTTACAACACTCCGGCGAG |
| 1055 | AGCAATCATGAGAGGTGGCCGGTG | CACCGGCCACCTCTCATGATTGCT |
| 1056 | ATTTGCCACCGGCGACAAAAAGAT | ATCTTTTTGTCGCCGGTGGCAAAT |
| 1057 | CCGCCCGTGTTGGCATGTCTTTTG | CAAAAGACATGCCAACACGGGCGG |
| 1058 | ATCGGAAGTGCTGACTGACACACG | CGTGTGTCAGTCAGCACTTCCGAT |
| 1059 | CCTCAGACCCTATCTGGGTTGACG | CGTCAACCCAGATAGGGTCTGAGG |
| 1060 | CTGTGTGGTCTGGTCCGGCTGTTC | GAACAGCCGGACCAGACCACACAG |
| 1061 | GTCCCCATTATCGGTGAGTGCAAC | GTTGCACTCACCGATAATGGGGAC |
| 1062 | ACAGGCACGTAAGTGCTCAATCGG | CCGATTGAGCACTTACGTGCCTGT |
| 1063 | AGCAAGATAGCGGGAGTGCCCCTA | TAGGGGCACTCCCGCTATCTTGCT |
| 1064 | GGTTTACGCCATGACATCCCGTCA | TGACGGGATGTCATGGCGTAAACC |
| 1065 | GTGCAGGCCTTTGTGTGTGAATCG | CGATTCACACACAAAGGCCTGCAC |
| 1066 | CTTCGAGGGTAGGGCTTCGAAACG | CGTTTCGAAGCCCTACCCTCGAAG |
| 1067 | AGTCGACACTTGGGTTTACCACGG | CCGTGGTAAACCCAAGTGTCGACT |
| 1068 | ACATAAATCTCGCCCGCTGCACTC | GAGTGCAGCGGGCGAGATTTATGT |
| 1069 | GTTTGGTTTTCCACGGAGGTTTGA | TCAAACCTCCGTGGAAAACCAAAC |
| 1070 | GCAGGAACCAGATTAGTGTCCCGG | CCGGGACACTAATCTGGTTCCTGC |
| 1071 | TTTGCTAGAGCGCGGAGCTAAAGC | GCTTTAGCTCCGCGCTCTAGCAAA |
| 1072 | CTATGTGGCATCGCTGACATGCTC | GAGCATGTCAGCGATGCCACATAG |
| 1073 | CCTAAGTCGGTTTGCAGCTGCTCT | AGAGCAGCTGCAAACCGACTTAGG |
| 1074 | GCGTTCGTCCACAGGAACGGAAGG | CCTTCCGTTCCTGTGGACGAACGC |
| 1075 | TAACCCGCGCCCGAGAAATTGTCT | AGACAATTTCTCGGGCGCGGGTTA |
| 1076 | TATGGTGCTCAGAGCTGTTGCCAA | TTGGCAACAGCTCTGAGCACCATA |
| 1077 | TCATCGACCCACTAACGTCAGGGC | GCCCTGACGTTAGTGGGTCGATGA |
| 1078 | TGCTCAAGCTACGCGTCACTTCCC | GGGAAGTGACGCGTAGCTTGAGCA |
| 1079 | AGCGGGAAGGTCTGAGGAGGGAAA | TTTCCCTCCTCAGACCTTCCCGCT |
| 1080 | CCGATGTAGCACCACCGCAGTGGC | GCCACTGCGGTGGTGCTACATCGG |
| 1081 | AAGTTCTGGGAATCACACGGCGCG | CGCGCCGTGTGATTCCCAGAACTT |
| 1082 | CACCAGCCTTACGTGCGGCGTTAA | TTAACGCCGCACGTAAGGCTGGTG |
| 1083 | CGTTTCGCCTCCTCTTCCGAATGC | GCATTCGGAAGAGGAGGCGAAACG |
| 1084 | GAGGAGGCCAATAGAGCAGCGCGC | GCGCGCTGCTCTATTGGCCTCCTC |
| 1085 | AGTAATCTTGCGGCACACAAGCGG | CCGCTTGTGTGCCGCAAGATTACT |
| 1086 | TGAGGACAAACCGCGCGTAGGATA | TATCCTACGCGCGGTTTGTCCTCA |
| 1087 | TCGTAGAGACGCAGTGCCCATCTC | GAGATGGGCACTGCGTCTCTACGA |
| 1088 | CGAAGCTACACCCCGAGTGCGGTG | CACCGCACTCGGGGTGTAGCTTCG |
| 1089 | ATGATGTGATCTTCCCATGGCTGG | CCAGCCATGGGAAGATCACATCAT |
| 1090 | TGTACACGTATCGCGTTCGCCTAG | CTAGGCGAACGCGATACGTGTACA |
| 1091 | GGTGTGCTTTTACGCATGTACGCA | TGCGTACATGCGTAAAAGCACACC |
| 1092 | AGGCGGGATACGTGGATGCTAGCC | GGCTAGCATCCACGTATCCCGCCT |
| 1093 | AAATTAGGCACAGCCCTCCCACAG | CTGTGGGAGGGCTGTGCCTAATTT |
| 1094 | ATAAGTTTGGTGAGCCATTCGCGA | TCGCGAATGGCTCACCAAACTTAT |
| 1095 | CCTATTTCGGCGGACCTCGATGCC | GGCATCGAGGTCCGCCGAAATAGG |
| 1096 | TTACCGGAATATGCACTTGGCCGC | GCGGCCAAGTGCATATTCCGGTAA |
| 1097 | CCTCTCGGACGGTCCCTTTGATCG | CGATCAAAGGGACCGTCCGAGAGG |
| 1098 | CAAGCGAATGCTGTATTACGGCCT | AGGCCGTAATACAGCATTCGCTTG |
| 1099 | GCATTTCCCATGCCAGAACGTTGA | TCAACGTTCTGGCATGGGAAATGC |
| 1100 | GTTTTGGCTAACCGTCCTGCCTTG | CAAGGCAGGACGGTTAGCCAAAAC |
| 1101 | AGGTTTTGTCCGGGCGAATGATGT | ACATCATTCGCCCGGACAAAACCT |
| 1102 | ATGTCCACGAGTGCGTCCGATATC | GATATCGGACGCACTCGTGGACAT |
| 1104 | AATACCGTTCCCATCTGTGCGAGG | CCTCGCACAGATGGGAACGGTATT |
| 1105 | ACACAAGGTGCCTCATCGAATGGT | ACCATTCGATGAGGCACCTTGTGT |
| 1106 | GCCGGCAAAATCCTACAAAATCCA | TGGATTTTGTAGGATTTTGCCGGC |
| 1107 | CTTATCCCATGTGCCGGTCTGACT | AGTCAGACCGGCACATGGGATAAG |
| 1108 | GCGGCCATAATGCATAGCACGGAA | TTCCGTGCTATGCATTATGGCCGC |
| 1109 | TACGGTGCATCGCAGTATGGGTAA | TTACCCATACTGCGATGCACCGTA |
| 1110 | CACCAGATGTCGAGGATCATCGCC | GGCGATGATCCTCGACATCTGGTG |
| 1111 | GCTCCTACGCCCAAAGAGGTATGG | CCATACCTCTTTGGGCGTAGGAGC |
| 1112 | AGAATATGGGCAGCAGCAGCACTC | GAGTGCTGCTGCTGCCCATATTCT |
| 1113 | CTGCAGTCGCACGCAGTAGACCCG | CGGGTCTACTGCGTGCGACTGCAG |
| 1114 | ATGTCCCTGACCGGAATCTTTCCA | TGGAAAGATTCCGGTCAGGGACAT |
| 1115 | TTCGCCACGAGGCATTAGTCCGAC | GTCGGACTAATGCCTCGTGGCGAA |
| 1116 | ACGTCGTTCCCGAGAATACGGTCT | AGACCGTATTCTCGGGAACGACGT |
| 1117 | ATCCGCTGGCGCTTTGACGAAGAA | TTCTTCGTCAAAGCGCCAGCGGAT |
| 1118 | TGAACCAAATTCTTACCGCGTGGA | TCCACGCGGTAAGAATTTGGTTCA |
| 1119 | CACGCGTAGGCTGGTGTGTCATTC | GAATGACACACCAGCCTACGCGTG |
| 1120 | TCGATCCCGCGATCTGGCCTATTG | CAATAGGCCAGATCGCGGGATCGA |
| 1121 | GGAACACTCAACCACCGTGGATCT | AGATCCACGGTGGTTGAGTGTTCC |
| 1122 | TCACACACCAACTGGCCACAGATG | CATCTGTGGCCAGTTGGTGTGTGA |
| 1123 | TGTGCTTAGGACACCAGGCAACCC | GGGTTGCCTGGTGTCCTAAGCACA |
| 1124 | GACATTTAACCCGACCGATTGTGC | GCACAATCGGTCGGGTTAAATGTC |
| 1125 | GGCACCGAGCCAGTAGGCCTCTGA | TCAGAGGCCTACTGGCTCGGTGCC |
| 1126 | CTCAAGCGTGCATGTTGGTAACCA | TGGTTACCAACATGCACGCTTGAG |
| 1127 | AGGAAGGCCACCATCCAATATTCG | CGAATATTGGATGGTGGCCTTCCT |
| 1128 | TACGAACGCCAAGGTTATGCCAAT | ATTGGCATAACCTTGGCGTTCGTA |
| 1129 | CGCACCAGAGTTATGCAGGCTCAA | TTGAGCCTGCATAACTCTGGTGCG |
| 1130 | CCAGCTTGGACGAGGAAGGATGTG | CACATCCTTCCTCGTCCAAGCTGG |
| 1131 | GTCACGCCTTTCAAATGACCCACA | TGTGGGTCATTTGAAAGGCGTGAC |
| 1132 | TGCTAGACCCAGCCCGAGTCTCGG | CCGAGACTCGGGCTGGGTCTAGCA |
| 1133 | TATTGTGGCACTTGGGTCCAGTGC | GCACTGGACCCAAGTGCCACAATA |
| 1134 | CACGTGTGAGACCGGAAGTGCATC | GATGCACTTCCGGTCTCACACGTG |
| 1135 | GGCAGCCTGATGCTACAGCACCGT | ACGGTGCTGTAGCATCAGGCTGCC |
| 1136 | CGGTGCGTCCATCCTTCAGAGTTA | TAACTCTGAAGGATGGACGGACCG |
| 1137 | CTATTCGCGGACCCTACGCAGTTT | AAACTGCGTAGGGTCCGCGAATAG |
| 1138 | ACCTGTGCAGTCAGCACGAGTGCG | CGCACTCGTGCTGACTGCACAGGT |
| 1139 | GAGAACCACAGGTGGTCCACCCTA | TAGGGTGGACCACCTGTGGTTCTC |
| 1140 | CCTCGCTAGAGAAATCCACGGGAT | ATCCCGTGGATTTCTCTAGCGAGG |
| 1141 | TAACATCGGTGCAAACCGTGGCGC | GCGCCACGGTTTGCACCGATGTTA |
| 1142 | ACCCAGAAGACATGGCATTCGCCT | AGGCGAATGCCATGTCTTCTGGGT |
| 1143 | AAAAGCGCTGCTCTAACACCGCCG | CGGCGGTGTTAGAGCAGCGCTTTT |
| 1144 | CAAGTCTGTCCATTTCCCAACGGT | ACCGTTGGGAAATGGACAGACTTG |
| 1145 | CCGACACATGGTGGGCTTTTTAAG | CTTAAAAAGCCCACCATGTGTCGG |
| 1146 | ACAGACCAGCTTTTTGCGCAGATT | AATCTGCGCAAAAAGCTGGTCTGT |
| 1147 | CGGCGATCCATTTCACTTCAAAGT | ACTTTGAAGTGAAATGGATCGCCG |
| 1148 | GACGTTATCATGACACAGGTCGCG | CGCGACCTGTGTCATGATAACGTC |
| 1149 | GGCAGAGTTGGATCGGATCCTCAA | TTGAGGATCCGATCCAACTCTGCC |
| 1150 | CCTCAATGCCACCGAATTCGGTAT | ATACCGAATTCGGTGGCATTGAGG |
| 1151 | GGAGTTAGCGTGATTAGTCGCCCA | TGGGCGACTAATCACGCTAACTCC |
| 1152 | GAACTCGACGTGTCACGGAAGGGT | ACCCTTCCGTGACACGTCGAGTTC |
| 1153 | CACAAGCGACATTTCTGGTGCACG | CGTGCACCAGAAATGTCGCTTGTG |
| 1154 | CCAGAATGCGTGAATTCGCGTCCT | AGGACGCGAATTCACGCATTCTGG |
| 1155 | CAAGGGAGCCCTGCGAATTAGAGT | ACTCTAATTCGCAGGGCTCCCTTG |
| 1156 | ATTCTTGCTTCGGACGACTAGCCG | CGGCTAGTCGTCCGAAGCAAGAAT |
| 1157 | TGCCACTTTGATTTCCAGATTGCC | GGCAATCTGGAAATCAAAGTGGCA |
| 1158 | GATGGTCGGCAGATAAGTGGTGGG | CCCACCACTTATCTGCCGACCATC |
| 1159 | GTTCACACGGGTTGACCAACATGT | ACATGTTGGTCAACCCGTGTGAAC |
| 1160 | GATTCAATTGCCCCATTCCTGCAT | ATGCAGGAATGGGGCAATTGAATC |
| 1161 | TACCGGAAACTGAGCCTCGTGCTA | TAGCACGAGGCTCAGTTTCCGGTA |
| 1162 | GGATCTTTACTCAGGGGCAGAGCC | GGCTCTGCCCCTGAGTAAAGATCC |
| 1163 | CGCGAGTGCTTTGTTCTGTGTGGA | TCCACACAGAACAAAGCACTCGCG |
| 1164 | GTCGTCGCGATGGCGTACATCCTT | AAGGATGTACGCCATCGCGACGAC |
| 1165 | ACGGGAATCTCCCGAAGTGCGAGC | GCTCGCACTTCGGGAGATTCCCGT |
| 1166 | GGTCGAAATGAGCCAGCAGCAGAT | ATCTGCTGCTGGCTCATTTCGACC |
| 1167 | CCATTGGAATACTGCGTGCGGCTT | AAGCCGCACGCAGTATTCCAATGG |
| 1168 | GGAAGACTTCGCGAGGGCACAATG | CATTGTGCCCTCGCGAAGTCTTCC |
| 1169 | AGGGTGACTTCGAAGGTCCGAACT | AGTTCGGACCTTCGAAGTCACCCT |
| 1170 | TCGTCCCTCTGGTGGTCGAATCAC | GTGATTCGACCACCAGAGGGACGA |
| 1171 | TGTGCAAATTATGCTGGGCGTGAG | CTCACGCCCAGCATAATTTGCACA |
| 1172 | GTCGCCAACTGTCATGTGTGCCCA | TGGGCACACATGACAGTTGGCGAC |
| 1173 | CCTCGAACCCTCAAGACGAAACGA | TCGTTTCGTCTTGAGGGTTCGAGG |
| 1174 | CTTCATCACGTGACCTTTGTTGCC | GGCAACAAAGGTCACGTGATGAAG |
| 1175 | CGTTCATTCCCAGCAGGATGGCTT | AAGCCATCCTGCTGGGAATGAAGG |
| 1176 | CGGGGACCTCAATGGAGCGTCTTA | TAAGACGCTCCATTGAGGTCCCCG |
| 1177 | CGCCTCTAGCGCTTGTTACGTCGA | TCGACGTAACAAGCGCTAGAGGCG |
| 1178 | CTGCCAGACTCAAAACAGGGACGG | CCGTCCCTGTTTTGAGTCTGGCAG |
| 1179 | CTCCTTACACCGTGTGAGGGAACC | GGTTCCCTCACACGGTGTAAGGAG |
| 1180 | TTTCATGCCATATCGCCTCGCGCA | TGCGCGAGGCGATATGGCATGAAA |
| 1181 | GTCTGACTGTCTGCCCTGTATGCG | CGCATACAGGGCAGACAGTCAGAC |
| 1182 | GGTTAATGGAACGGCGTTAACGCG | CGCGTTAACGCCGTTCCATTAACC |
| 1183 | CTTCGCACTGCGGAATCTCAAGCT | AGCTTGAGATTCCGCAGTGCGAAG |
| 1184 | TGCCAGAGGCGTAGGAGTCCTGGA | TCCAGGACTCCTACGCCTCTGGCA |
| 1185 | GACGGGCGAGCCAGTATTAACTCA | TGAGTTAATACTGGCTCGCCCGTC |
| 1186 | GACCTCCAAAGTCAGTCTTGGCGG | CCGCCAAGACTGACTTTGGAGGTC |
| 1187 | CGTTAGAGCATGACCGAACACGTC | GACGTGTTCGGTCATGCTCTAACG |
| 1188 | GTGGGCTCAAAAATTGGGTACGCC | GGCGTACCCAATTTTTGAGCCCAC |
| 1189 | GGGGCAGAGATCACGCGTTCCTCT | AGAGGAACGCGTGATCTCTGCCCC |
| 1190 | TTTCGCCCTACGAAGCGAAGTTTC | GAAACTTCGCTTCGTAGGGCGAAA |
| 1191 | TACGGGGTGATGTTAAGCTACGCG | CGCGTAGCTTAACATCACCCCGTA |
| 1192 | CCTGTGAGTCTGAGATCGCCGTGT | ACACGGCGATCTCAGACTCACAGG |
| 1193 | ACTGAAGCTGGAACAGGCCATTCG | CGAATGGCCTGTTCCAGCTTCAGT |
| 1194 | AGCACTGGTTCACATGGGAGTCCA | TGGACTCCCATGTGAACCAGTGCT |
| 1195 | TAAGGAAGATCACACTCCCTGCGC | GCGCAGGGAGTGTGATCTTCCTTA |
| 1196 | CACCACACGCTAAAATTGAAGCCG | CGGCTTCAATTTTAGCGTGTGGTG |
| 1197 | GCTGTCGCCAGGATCATGTATCGT | ACGATACATGATCCTGGCGACAGC |
| 1198 | TTCGTTCGTGCACTGGATTCTTGA | TCAAGAATCCAGTGCACGAACGAA |
| 1199 | TCAGCTCTCCTTGTGCTTGCAGTG | CACTGCAAGCACAAGGAGAGCTGA |
| 1200 | ACGACGAGGTGAACTTCGTGGGAA | TTCCCACGAAGTTCACCTCGTCGT |
| 1201 | AGCATTGCCGCGGGCCTTGGTTTA | TAAACCAAGGCCCGCGGCAATGCT |
| 1202 | CAGAGGGCAGATGTGACTCCTCAA | TTGAGGAGTCACATCTGCCCTCTG |
| 1203 | CGATATTTCAGCCTCTCAAACGCG | CGCGTTTGAGAGGCTGAAATATCG |
| 1204 | TGCCAGAAATGTTGCCGATTCGAA | TTCGAATCGGCAACATTTCTGGCA |
| 1205 | TAGGCCACCCGGTGTTCACAATTC | GAATTGTGAACACCGGGTGGCCTA |
| 1206 | GAGAGTCAGACCGAGGGACACGAG | CTCGTGTCCCTCGGTCTGACTCTC |
| 1207 | GAGGCGATCCTGGAACCACGCAAC | GTTGCGTGGTTCCAGGATCGCCTC |
| 1208 | CCAGAGAGGCGGGCTACTGACTCA | TGAGTCAGTAGCCCGCCTCTCTGG |
| 1209 | CACACAGTCCCATCGTACGGCAGT | ACTGCCGTACGATGGGACTGTGTG |
| 1210 | TTACGTTGCGGAAGCGTGCCTCTA | TAGAGGCACGCTTCCGCAACGTAA |
| 1211 | ATGTACACGCTGCAATCGTGTCCC | GGGACACGATTGCAGCGTGTACAT |
| 1212 | ACTCGTCGTCGGAAGCGCCCAGGT | ACCTGGGCGCTTCCGACGACGAGT |
| 1213 | ATGCGAGAGCAGAATTGAGCCGGT | ACCGGCTCAATTCTGCTCTCGCAT |
| 1214 | AAGTTGGTTCGTATTCACGCGTGC | GCACGCGTGAATACGAACCAACTT |
| 1215 | TGGGCTTATCGCCGAAGATTGCTA | TAGCAATCTTCGGCGATAAGCCCA |
| 1216 | CAACGGCGAAGACCCAGAATTTTA | TAAAATTCTGGGTCTTCGCCGTTG |
| 1217 | AGCGTACGGCGAAAGTCTAGGGAC | GTCCCTAGACTTTCGCCGTACGCT |
| 1218 | ATGCATCCAGCGTCCCCTTGATTA | TAATCAAGGGGACGCTGGATGCAT |
| 1219 | ACCGTCATCAGTCGCAGGCTTCTG | CAGAAGCCTGCGACTGATGACGGT |
| 1220 | TCTTGACGGCTGGGCATGNTTGGA | TCCAATCATGCCCAGCCGTCAAGA |
| 1221 | TTAACATTCGGACCCAGGACCTGG | CCAGGTCCTGGGTCCGAATGTTAA |
| 1222 | TGGTGTCGAACTCCCTTGCGTGTT | AACACGCAAGGGAGTTCGACACCA |
| 1223 | TACTCCAGTCGCCTGCGCGCAATC | GTTTGCGCGCAGGCGACTGGAGTA |
| 1224 | CGCAATGCCGTAAGCATGCCAAGC | GCTTGGCATGCTTACGGCATTGCG |
| 1225 | AGTCCGCGCGAAATACGAACAGTA | TACTGTTCGTATTTCGCGCGGACT |
| 1226 | ATGTTGCACGCGCACTGTATCACA | TGTGATACAGTGCGCGTGCAACAT |
| 1227 | ATCGCCTAACTACCCGCGGCGTGC | GCACGCCGCGGGTAGTTAGGCGAT |
| 1228 | TGGCCAGGGAACACAAGCTCGGTA | TACCGAGCTTGTGTTCCCTGGCCA |
| 1229 | AAACATGGGTCGCGTCTGAGATCA | TGATCTCAGACGCGACCCATGTTT |
| 1230 | GCGAGAGCTGCGATTCCCTTTTAG | CTAAAAGGGAATCGCAGCTCTCGC |
| 1231 | CCGGCCAAACAAGAGACGAGCGGA | TCCGCTCGTCTCTTGTTTGGCCGG |
| 1232 | AATGGGGCACAGTCTCGCTTGACA | TGTCAAGCGAGACTGTGCCCCATT |
| 1233 | TGTCTCGGGCCTTCAGGACACACT | AGTGTGTCCTGAAGGCCCGAGACA |
| 1234 | TCCACCTTCATTAAGTGGTTCGGC | GCCGAACCACTTAATGAAGGTGGA |
| 1235 | GCTTCGGAATCATCCACCTGTCAT | ATGACAGGTGGATGATTCCGPAGC |
| 1236 | GAGCCGATGGGCTATCGTCGTCGG | CCGACGACGATAGCCCATCGGCTC |
| 1237 | CACGAATTACGCACGCACAGAGGA | TCCTCTGTGCGTGCGTAATTCGTG |
| 1238 | GCTGTGACGCTCCCCTCAACTAGG | CCTAGTTGAGGGGAGCGTCACAGC |
| 1239 | CGCTCTGAAAACGCGGGCTACGTT | AACGTAGCCCGCGTTTTCAGAGCG |
| 1240 | GAGTGCTGGACACCGTAGCCAGGA | TCCTGGCTACGGTGTCCAGCACTC |
| 1241 | CCAACCCCAGTGTAGGCGCAAATG | CATTTGCGCCTACACTGGGGTTGG |
| 1242 | GAAGTAGGGGATGTTGGCCGGCGG | CCGCCGGCCAACATCCCCTACTTC |
| 1243 | CAACGTGGGCACCTGTTTTAGCAG | CTGCTAAAACAGGTGCCCACGTTG |
| 1244 | CTAGCTGCGATCCGAACCTCTACG | CGTAGAGGTTCGGATCGCAGCTAG |
| 1245 | CATTGAACCATCAGCCAAGCTGCG | CGCAGCTTGGCTGATGGTTCAATG |
| 1246 | AGACTGGCAATTTTTCGAGGCCAA | TTGGCCTCGAAAAATTGCCAGTCT |
| 1247 | CTGGCCGTCCATGAGTTGGTCCAG | CTGGACCAACTCATGGACGGCCAG |
| 1248 | CATGCTGAAACACGGGATTGCCAT | ATGGCAATCCCGTGTTTCAGCATG |
| 1249 | CGATATGTAAGACAGCCGTCGCAA | TTGCGACGGCTGTCTTACATATCG |
| 1250 | AGCGTAACCTACTGGGAAGGCACC | GGTGCCTTCCCAGTAGGTTACGCT |
| 1251 | GTTCGAACCCCGCGATGTTAAATG | CATTTAACATCGCGGGGTTCGAAC |
| 1252 | GTTGTTAGGAGGCTCGAGGCTGCT | AGCAGCCTCGAGCCTCCTAACAAC |
| 1253 | ACTGGTGCTACGCGGGATATTTGA | TCAAATATCCCGCGTAGCACCAGT |
| 1254 | CTGGGAGCTATCCTCAGCCGAATC | GATTCGGCTGAGGATAGCTCCCAG |
| 1255 | GAACTCGCCGCTGCCGAAGGGTAG | CTACCCTTCGGCAGCGGCGAGTTC |
| 1256 | TTCGATCGAGGAGCAAGGAGAGTC | GACTCTCCTTGCTCCTCGATCGAA |
| 1257 | GGGGAAAATTGAGGCCTTAGCCAT | ATGGCTAAGGCCTCAATTTTCCCC |
| 1258 | CTAAGGTCAAAGCGCTGTCGCCAG | CTGGCGACAGCGCTTTGACCTTAG |
| 1259 | CCGTAGCGGTGCTCGACCAGGTTC | GAACCTGGTCGAGCACCGCTACGG |
| 1260 | TGGGGACGAATCCGAATGTAGTGA | TCACTACATTCGGATTCGTCCCCA |
| 1261 | GTCATGTAATTGCATCCCACGGGT | ACCCGTGGGATGCAATTACATGAC |
| 1262 | CTTTGCGCGGTGGTCAATAAAAAG | CTTTTTATTGACCACCGCGCAAAG |
| 1263 | CTCGGGGATGCCCTCTTGGCATTA | TAATGCCAAGAGGGCATCCCCGAG |
| 1264 | CGAAACGTGGTGCAGAAACCTGAA | TTCAGGTTTCTGCACCACGTTTCG |
| 1265 | GGAGTTCACGAGTCGAGCAGTCGC | GCGACTGCTCGACTCGTGAACTCC |
| 1266 | AGCCGTTTTCAAAGATCTCGACGA | TCGTCGAGATCTTTGAAAACGGCT |
| 1267 | TGGCTGGACATTGTCTGCAATGCA | TGCATTGCAGACAATGTCCAGCCA |
| 1268 | ATCGGCTGCCTCAGTCCCTAATTT | AAATTAGGGACTGAGGCAGCCGAT |
| 1269 | CCAGCATGGAGTTAAGTGAGCGCG | CGCGCTCACTTAACTCCATGCTGG |
| 1270 | TTCATATTTACGAATGCCGGGTGC | GCACCCGGCATTCGTAAATATGAA |
| 1271 | CGAAATCGCACAGGAATTCGCGTC | GACGCGAATTCCTGTGCGATTTCG |
| 1272 | GGCAATTTCGGGACACTCGTTTCA | TGAAACGAGTGTCCCGAAATTGCC |
| 1273 | TTTGTGATTGGGGGTATAACCCGA | TCGGGTTATACCCCCAATCACAAA |
| 1274 | CCCAGCTAATCCAGCTTGGGCTGT | ACAGCCCAAGCTGGATTAGCTGGG |
| 1275 | AAAATCGTTTGGCTGTAACGTCGC | GCGACGTTACAGCCAAACGATTTT |
| 1276 | AGGAGATTCATCGACTTCCGGGAA | TTCCCGGAAGTCGATGAATCTCCT |
| 1277 | GCACGGGGTCTCAATGCTTAGGGT | ACCCTAAGCATTGAGACCCCGTGC |
| 1278 | GCGCAACAAGTAGCCTACCGAGGC | GCCTCGGTAGGCTACTTGTTGCGC |
| 1279 | TAGCAGGCTGATGCCGTCTACACA | TGTGTAGACGGCATCAGCCTGCTA |
| 1280 | GCAAGCGGCGATCGTACAACTTGT | ACAAGTTGTACGATCGCCGCTTGC |
| 1281 | GCACCTCTGGTAAGCCTGAAAGGG | CCCTTTCAGGCTTACCAGAGGTGC |
| 1282 | CGAGGGCGGTGAGTGCATACCGTG | CACGGTATGCACTCACCGCCCTCG |
| 1283 | GGATTAACCGGAACTGCCCTTCTG | CAGAAGGGCAGTTCCGGTTAATCC |
| 1284 | GATATTGGGTCCGGCGCGCATTAC | GTAATGCGCGCCGGACCCAATATC |
| 1285 | GGCCTTTAATCTCCGGTCGCAATG | CATTGCGACCGGAGATTAAAGGCC |
| 1286 | AACCTTAGTGCGGCTAGGTGGGGT | ACCCCACCTAGCCGCACTAAGGTT |
| 1287 | CACGCTGACGCCAGTGTGGTGAGG | CCTCACCACACTGGCGTCAGCGTG |
| 1288 | GGTTCCCTTGACCCACCGAATTGA | TCAATTCGGTGGGTCAAGGGAACC |
| 1289 | TTCTGACAACATCGACCCTGGCTC | GAGCCAGGGTCGATGTTGTCAGAA |
| 1290 | GCGAGCGAAGATAATCCCCAAACT | AGTTTGGGGATTATCTTCGCTCGC |
| 1291 | GTACTCTGTGCAACGGTCCCGAGT | ACTCGGGACCGTTGCACAGAGTAC |
| 1292 | ACACGCCAGGAACAGTGTCTGTGA | TCACAGACACTGTTCCTGGCGTGT |
| 1293 | AAGGGAATTTAGCGCGCGTGACTT | AAGTCACGCGCGCTAAATTCCCTT |
| 1294 | TGACGTACGCGTTTTAAGTGGGGA | TCCCCACTTAAAACGCGTACGTCA |
| 1295 | CTTAGAGGGACGAGGCCATGAATG | CATTCATGGCCTCGTCCCTCTAAG |
| 1296 | GGACGACTCCGCAAAAAAGGTCGT | ACGACCTTTTTTGCGGAGTCGTCC |
| 1297 | TCAATCCCAACATCCAAAGCCTCA | TGAGGCTTTGGATGTTGGGATTGA |
| 1298 | GCACTGGTCTACCAAGCTTGTCCC | GGGACAAGCTTGGTAGACCAGTGC |
| 1299 | ACTTGTCGGAAACGAGACCGAGCA | TGCTCGGTCTCGTTTCCGACAAGT |
| 1300 | TCAGGAAAGGCCTAAAGGCGAAAG | CTTTCGCCTTTAGGCCTTTCCTGA |
| 1301 | GGAATGTAGTCAAGGAGGACGGGG | CCCCGTCCTCCTTGACTACATTCC |
| 1302 | GCACGTGGTAAATGAATTGGCGAG | CTCGCCAATTCATTTACCACGTGC |
| 1303 | GATCATCAGGGGTTATGCGTCGCG | CGCGACGCATAACCCCTGATGATC |
| 1304 | CTCACTCATTCTGATTGCCCGCGG | CCGCGGGCAATCAGAATGAGTGAG |
| 1305 | GGGGTGATCTCTCGAACGTCACCC | GGGTGACGTTCGAGAGATCACCCC |
| 1306 | AAGGTTGCTGCTAGCGTACCTCGA | TCGAGGTACGCTAGCAGCAACCTT |
| 1307 | TATAGATCGCCCAACAGGCAGGAG | CTCCTGCCTGTTGGGCGATCTATA |
| 1308 | GTTTGGACCTGTTGGGAGTGGGCA | TGCCCACTCCCAACAGGTCCAAAC |
| 1309 | ATTGGGGAAAACCCGGTCTCAAGG | CCTTGAGACCGGGTTTTCCCCAAT |
| 1310 | TCGACGATAAAGTGCTCACGGGAC | GTCCCGTGAGCACTTTATCGTCGA |
| 1311 | CGATAGAATTCAATGCAGGGCGGA | TCCGCCCTGCATTGAATTCTATCG |
| 1312 | CGGTTCGCTACGGCGGCTGGTTTC | GAAACCAGCCGCCGTAGCGAACCG |
| 1313 | CCAGGTTTCGGTTAGTCGCGCTAG | CTAGCGCGACTAACCGAAACCTGG |
| 1314 | ACGACCTTACACTCGGATCCGACG | CGTCGGATCCGAGTGTAAGGTCGT |
| 1315 | TCGCGTTAAATGGACCAAGGGGCC | GGCCCCTTGGTCCATTTAACGCGA |
| 1316 | CCAGAAAGAAAATGGCGCCCGGAT | ATCCGGGCGCCATTTTCTTTCTGG |
| 1317 | GATACATCGCCGCCTGCTAGGCAC | GTGCCTAGCAGGCGGCGATGTATC |
| 1318 | GAGATCACACTCGGAAACCGGATG | CATCCGGTTTCCGAGTGTGATCTC |
| 1319 | ACTTCGCGGAAAAAGGCTGGCATT | AATGCCAGCCTTTTTCCGCGAAGT |
| 1320 | CCGAGCTGCACGAGCACACAAAGT | ACTTTGTGTGCTCGTGCAGCTCGG |
| 1321 | TTCCACAAGGCGGCATAGTGAGGC | GCCTCACTATGCCGCCTTGTGGAA |
| 1322 | AGCAAACTGGAATCCGGAAAAACC | GGTTTTTCCGGATTCCAGTTTGCT |
| 1323 | CGCTATGTCGCAGCATGCATTTAC | GTAAATGCATGCTGCGACATAGCG |
| 1324 | AGTCACGCCCAACGTCGGTTCTTT | AAAGAACCGACGTTGGGCGTGACT |
| 1325 | AGTGGGCGCACTTGGCCTTAAATA | TATTTAAGGGGAAGTGCGCCCACT |
| 1326 | ACTTGCAACTTCGGCCGTTTGACT | AGTCAAACGGCCGAAGTTGCAAGT |
| 1327 | CAAACATCAGGTTCATGCCGTACG | CGTACGGCATGAACCTGATGTTTG |
| 1328 | AGCGTGACCACCCTACAATGGCAA | TTGCCATTGTAGGGTGGTCACGCT |
| 1329 | GCAGGCATCCGGCAGAGATGTCTC | GAGACATCTCTGCCGGATGCCTGC |
| 1330 | GAGCGGCTAAGAGGCCAGACCAAA | TTTGGTCTGGCCTCTTAGCCGCTC |
| 1331 | CACAGAACAGGGTGTTTCCCGCTA | TAGCGGGAAACACCCTGTTCTGTG |
| 1332 | ACTTTGCAGAAGGCCCAACACAAG | CTTGTGTTGGGCCTTCTGCAAAGT |
| 1333 | CCTTCCTGGTACTTTGTGGGCGAC | GTCGCCCACAAAGTACCAGGAAGG |
| 1334 | CTACATGCTCACCCCACCAGAGTG | CACTCTGGTGGGGTGAGCATGTAG |
| 1335 | ATTTTCAGAATAGCCCCGCCTCGA | TCGAGGCGGGGCTATTCTGAAAAT |
| 1336 | CAATTGCTACGTTGACGCCCTCTG | CAGAGGGCGTCAACGTAGCAATTG |
| 1337 | CTGTCGCCTAATCCTCGGTGGCCG | CGGCCACCGAGGATTAGGCGACAG |
| 1338 | TTTGTGTTGGCTCCGTACATTGGA | TCCAATGTACGGAGCCAACACAAA |
| 1339 | ACGTGACGGGAAGGTGGTTGAATC | GATTCAACCACCTTCCCGTCACGT |
| 1340 | AGTTCTTGCGTTGCACGAAACAGA | TCTGTTTCGTGCAACGCAAGAACT |
| 1341 | GCTCGCCGCGCGTCTTTATGTCTG | CAGACATAAAGACGCGCGGCGAGC |
| 1342 | ATGAACATCGCGAGGCAAGCCTTT | AAAGGCTTGCCTCGCGATGTTCAT |
| 1343 | CAACCGCGCCCACCAACATTAAGG | CCTTAATGTTGGTGGGCGCGGTTG |
| 1344 | TGATCGAGGACGGCTTGGTAGCCT | AGGCTACCAAGCCGTCCTCGATCA |
| 1345 | GGAGGCATGCCTTCCGAGAGCAAC | GTTGCTCTCGGAAGGCATGCCTCC |
| 1346 | CACCGATCCTCAACGCAATTGCTA | TAGCAATTGCGTTGAGGATCGGTG |
| 1347 | GGCCATGAATTGGGAAATCCATGT | ACATGGATTTCCCAATTCATGGCC |
| 1348 | CTGTTCCAGGCGTAACCAGCGGGC | GCCCGCTGGTTACGCCTGGAACAG |
| 1349 | TATGTCTGGCTCGCCATCAGAAGA | TCTTCTGATGGCGAGCCAGACATA |
| 1350 | GGAGTGACCAGCACAAGCATCGAG | CTCGATGCTTGTGCTGGTCACTCC |
| 1351 | TCGGACTGGAAGTAACTCGCATGA | TCATGCGAGTTACTTCCAGTCCGA |
| 1352 | GTAGGGTCAAGCACGATTGAAGCC | GGCTTCAATCGTGCTTGACCCTAC |
| 1353 | CACCGGCGGTTCGACTAACGTGAC | GTCACGTTAGTCGAACCGCCGGTG |
| 1354 | GAATGACGCGCAGTGCATTTGAAC | GTTCAAATGCACTGCGCGTCATTC |
| 1355 | GTGCTCGTCTAACCGCGGATAGAG | CTCTATCCGCGGTTAGACGAGCAC |
| 1356 | GCGGACCTGGGTTAATTGACGCGC | GCGCGTCAATTAACCCAGGTCCGC |
| 1357 | TTTTTGATGTTGCGCACCGGGCTA | TAGCCCGGTGCGCAACATCAAAAA |
| 1358 | TTGCGTCAGCGCATCTGCTCGATT | AATCGAGCAGATGCGCTGACGCAA |
| 1359 | ATGAGCACGCCAGTTCGTTCCTTT | AAAGGAACGAACTGGCGTGCTCAT |
| 1360 | TCAACGGTAAAGAATCGCCCCGCA | TGCGGGGCGATTCTTTACCGTTGA |
| 1361 | CGCGATTGACTGAACCACACCTCT | AGAGGTGTGGTTCAGTCAATCGCG |
| 1362 | GCGTGAAAGATGACGGCCGGTATA | TATACCGGCCGTCATCTTTCACGC |
| 1363 | CATGATTCCACCTCGATCGGCTAG | CTAGCCGATCGAGGTGGAATCATG |
| 1364 | CTACGACAAAGCAACCGTGCAAAA | TTTTGCACGGTTGCTTTGTCGTAG |
| 1365 | ATGCCGTGTTCATCTTGATGGTCC | GGACCATCAAGATGAACACGGCAT |
| 1366 | TTCGTGGAGGGACTTTGGAGATCC | GGATCTCCAAAGTCCCTCCACGAA |
| 1367 | GAAGCGCCGTAACGTACACCGTCG | CGACGGTGTACGTTACGGCGCTTC |
| 1368 | AGCGTGCGCTTGGCTATAAGGCTA | TAGCCTTATAGCCAAGCGCACGCT |
| 1369 | ACAGTCAGGAGTAACGCCGCTCAA | TTGAGCGGCGTTACTCCTGACTGT |
| 1370 | TTTAGCCGCTGCGACTGTAGGAAA | TTTCCTACAGTCGCAGCGGCTAAA |
| 1371 | ACTGTGTCGCAATCAACCCGCAAA | TTTGCGGGTTGATTGCGACACAGT |
| 1372 | TGCAGCCAATGCGGAACTTAGAGG | CCTCTAAGTTCCGCATTGGCTGCA |
| 1373 | CCCGCTATCCCGGTCTTGCAGTTC | GAACTGCAAGACCGGGATAGCGGG |
| 1374 | GAGGGCGCAACATATGCAGTGCTG | CAGCACTGCATATGTTGCGCCCTC |
| 1375 | CGTACGGACATCGATGACGCAACG | CGTTGCGTCATCGATGTCCGTACG |
| 1376 | AGTCTCCCGAGAAACGCATAAGGC | GCCTTATGCGTTTCTCGGGAGACT |
| 1377 | AGGAAGTGGATGAACGCGGCTGCA | TGCAGCCGCGTTCATCCACTTCCT |
| 1378 | GGGTTGCTCACCCTCGTCATCAGG | CCTGATGACGAGGGTGAGCAACCC |
| 1379 | TAGGAATGCGAGTTCCGGCGGTAA | TTACCGCCGGAACTCGCATTCCTA |
| 1380 | CTCCTCACTTCCAAGCTGCGGATA | TATCCGCAGCTTGGAAGTGAGGAG |
| 1381 | TCAATAGCACCTAGCATGCTCCCG | CGGGAGCATGCTAGGTGCTATTGA |
| 1382 | TGATTCCTGCGCTTTCACAGGTCG | CGACCTGTGAAAGCGCAGGAATCA |
| 1383 | GTATGTGCGGGATGGAAATCACGC | GCGTGATTTCCATCCCGCACATAC |
| 1384 | TACGGCAACTGTCGATACGAGGGC | GCCCTCGTATCGACAGTTGCCGTA |
| 1385 | GGTTCCCTATCCAGCACTCCTCGC | GCGAGGAGTGCTGGATAGGGAACC |
| 1386 | ATAAGCGCGCCACAGGTATGTACC | GGTACATACCTGTGGCGCGCTTAT |
| 1387 | GAAAGTCGCCAACAGACTCGAGCA | TGCTCGAGTCTGTTGGCGACTTTC |
| 1388 | CGCTAATGCCTCATAGGCGTGTGC | GCACACGCCTATGAGGCATTAGCG |
| 1389 | ATCCCCGCCGCACGAAGTACCAAG | CTTGGTACTTCGTGCGGCGGGGAT |
| 1390 | GACGCTGCTGATGGCTTTATCGAT | ATCGATAAAGCCATCAGCAGCGTC |
| 1391 | CTCTCCCCGTCGCTTCAGAGATTA | TAATCTCTGAAGCGACGGGGAGAG |
| 1392 | TCATGTGGGCCGTCGTATCAGTTT | AAACTGATACGACGGCCCACATGA |
| 1393 | GGCCTGAAGGTGAATGGTTACGTG | CACGTAACCATTCACCTTCAGGCC |
| 1394 | AGCCTCCAAAGCCGGTAGAGTTCC | GGAACTCTACCGGCTTTGGAGGCT |
| 1395 | TTGTCGTAGGCGCTCACCTTAGGA | TCCTAAGGTGAGCGCCTACGACAA |
| 1396 | GCCTGAGTCCGGGTCGGGAAAGAA | TTCTTTCCCGACCCGGACTCAGGC |
| 1397 | GGCACTATACCGGTTCTGGACGCG | CGCGTCCAGAACCGGTATAGTGCC |
| 1398 | CCGTGTATACGGAAAGGTACGCCA | TGGCGTACCTTTCCGTATACACGG |
| 1399 | CCCAAGGCAAGTGTGCATCAGTCC | GGACTGATGCACACTTGCCTTGGG |
| 1400 | GGAGTGCATCATGGCCAAATCTGG | CCAGATTTGGCCATGATGCACTCC |
| 1401 | CCATGTTACGTCTGCGCACCACAG | CTGTGGTGCGCAGACGTAACATGG |
| 1402 | GGCGTTGAGCTTAAAAGCAGCGAC | GTCGCTGCTTTTAAGCTCAACGCC |
| 1403 | TTGGCACTCTGCAAGATACGTGGG | CCCACGTATCTTGCAGAGTGCCAA |
| 1404 | GATCTGCACTGCAAGGTCTTGGGG | CCCCAAGACCTTGCAGTGCAGATC |
| 1405 | CGATCAACTTGCGGCCATTCCTGC | GCAGGAATGGCCGCAAGTTGATCG |
| 1406 | CGGCTGGGGTCACAGAAACGAGTA | TACTCGTTTCTGTGACCCCAGCCG |
| 1407 | GCGGCTAGTTGTACCTAGCGGCTG | CAGCCGCTAGGTACAACTAGCCGC |
| 1408 | TCGTCACTGTTAGAGAGGCCTCCG | CGGAGGCCTCTCTAACAGTGACGA |
| 1409 | AGTGTCGTGAGCCCTAGCGGCGCT | AGCGCCGCTAGGGCTCACGACACT |
| 1410 | AGGACGCAGGGATTCAAGTGCAAC | GTTGCACTTGAATCCCTGCGTCCT |
| 1411 | ACCGATGCGCGGTCGGTCTCATAC | GTATGAGACCGACCGCGCATCGGT |
| 1412 | GGCAGAGGGTTAGGGGGTTTTTTT | AAAAAAACCCCCTAACCCTCTGCC |
| 1413 | GGCAAAGGGTGTTTATGGGAGACC | GGTCTCCCATAAACACCCTTTGCC |
| 1414 | ACAAGGCTTCGGCTGGCAGAATAC | GTATTCTGCCAGCCGAAGCCTTGT |
| 1415 | CATATCCGTTCCTATCGCCAGACG | CGTCTGGCGATAGGAACGGATATG |
| 1416 | AAGCCTTTGTGGCCAAGGCCGCGT | ACGCGGCCTTGGCCACAAAGGCTT |
| 1417 | CCGAACCATGGCTTTATCCAGTGT | ACACTGGATAAAGCCATGGTTCGG |
| 1418 | GTTCAGCAGTAGCTCCCTCCTCGA | TCGAGGAGGGAGCTACTGCTGAAC |
| 1419 | GCGCAGTGACACCATGATGCTTTC | GAAAGCATCATGGTGTCACTGCGC |
| 1420 | ACGATCCATTTTGCCAGCATGCAA | TTGCATGCTGGCAAAATGGATCGT |
| 1421 | TCCCTTCATTTCGGGTTTTTAGCC | GGCTAAAAACCCGAAATGAAGGGA |
| 1422 | TCTTCTTGCCCACATTCCCTTTTG | CAAAAGGGAATGTGGGCAAGAAGA |
| 1423 | TGCCTTTTGATTGGTGGTCACGGT | ACCGTGACCACCAATCAAAAGGCA |
| 1424 | GACCCTCACGGTCATCAGAGGGAG | CTCCCTCTGATGACCGTGAGGGTC |
| 1425 | CCGTTCAACACAGTGATACACGCG | CGCGTGTATCACTGTGTTGAACGG |
| 1426 | CACCAGGGGATAGGTGCGGTACGC | GCGTACCGCACCTATCCCCTGGTG |
| 1427 | GGTCGGAACTGATCTGTGCGATCC | GGATCGCACAGATCAGTTCCGACC |
| 1428 | TGCTCCTTCCTAGGGTCATCCGTG | CACGGATGACCCTAGGAAGGAGCA |
| 1429 | GTGGACTTTGACGCCGGCTACCGC | GCGGTAGCCGGCGTCAAAGTCCAC |
| 1430 | CTGATCTGTCGGCGGTTACTTGCC | GGCAAGTAACCGCCGACAGATCAG |
| 1431 | AGAGGAGCGGAAAAAACCGGACGA | TCGTCCGGTTTTTTCCGCTCCTCT |
| 1432 | GCGACGAAGAGATCCAGCAAGCTC | GAGCTTGCTGGATCTCTTCGTCGC |
| 1433 | GGGACTTCCAGCTGAGGGACGAAA | TTTCGTCCCTCAGCTGGAAGTCCC |
| 1434 | GGCGCACTCCAATACCCACTGTTT | AAACAGTGGGTATTGGAGTGCGCC |
| 1435 | GCGCTTGGAGACTGTCAGGACGTG | CACGTCCTGACAGTCTCCAAGCGC |
| 1436 | CAAACCGCTGGTTTCTCCACCTGT | ACAGGTGGAGAAACCAGCGGTTTG |
| 1437 | GCGATTGCTTGGGATCGGTGACTA | TAGTCACCGATCCCAAGCAATCGC |
| 1438 | CTCAGCGACATTTTTCTGGTGGCG | CGCCACCAGAAAAATGTCGCTGAG |
| 1439 | CAGCGGCGTCGTTTACTCAGGACT | AGTCCTGAGTAAACGACGCCGCTG |
| 1440 | GACAGCCGTGAACGCTCAGCCGTT | AACGGCTGAGCGTTCACGGCTGTC |
| 1441 | GGGCCGTAGAGGCATCGGGTAAAG | CTTTACCCGATGCCTCTACGGCCC |
| 1442 | CGCCGCTCACCTGCTTAAAGCATT | AATGCTTTAAGCAGGTGAGCGGCG |
| 1443 | TGCCAAATCGCAACTCTTGAGACA | TGTCTCAAGAGTTGCGATTTGGCA |
| 1444 | CCCCGATCGGGTGTAATTCTCCCT | AGGGAGAATTACACCCGATCGGGG |
| 1445 | CAAGGTCCAGGTGACGCAACCACT | AGTGGTTGCGTCACCTGGACCTTG |
| 1446 | CGAGCCTTCAGTGGTATGCATGCG | CGCATGCATACCACTGAAGGCTCG |
| 1447 | CAGCAGCGTGCCCATCTCGACTTA | TAAGTCGAGATGGGCACGCTGCTG |
| 1448 | CGGACCAAGATGGCAGTAATCCAG | CTGGATTACTGCCATCTTGGTCCG |
| 1449 | CTACCACGCTCTGCGCGGGCTGTA | TACAGCCCGCGCAGAGCGTGGTAG |
| 1450 | ACGTGGTTAGGCATGAGCTGCGTC | GACGCAGCTCATGCCTAACCACGT |
| 1451 | CGACATATCCGACATGACCGGATG | CATCCGGTCATGTCGGATATGTCG |
| 1452 | GCGCCCAGGCTGTGTTAGAAAATA | TATTTTCTAACACAGCCTGGGCGC |
| 1453 | AGCTGGGACTCCGGACCTTGAGTG | CACTCAAGGTCCGGAGTCCCAGCT |
| 1454 | CGGTCGTAACCGCTGCTACAACTT | AAGTTGTAGCAGCGGTTACGACCG |
| 1455 | TCGTTCCTCTGGAACAATTCAGCA | TGCTGAATTGTTCCAGAGGAACGA |
| 1456 | CGGCATCTCCGGACAAAGGTTAAC | GTTAACCTTTGTCCGGAGATGCCG |
| 1457 | TATCTTGTCGAGCGCCACTCGGAG | CTCCGAGTGGCGCTCGACAAGATA |
| 1458 | TGCAAGGGAGAAAGCCCCATGAGC | GCTCATGGGGCTTTCTCCCTTGCA |
| 1459 | ACTGCATAGCCCAGATCCGCTTGC | GCAAGCGGATCTGGGCTATGCAGT |
| 1460 | TGTGATTCAGTCGAAGCAAGGCCG | CGGCCTTGCTTCGACTGAATCACA |
| 1461 | CATCCATCTACAATTCGGGCCAGT | ACTGGCCCGAATTGTAGATGGATG |
| 1462 | ATGAGCCGTTCAGAAAGCCAAAGA | TCTTTGGCTTTCTGAACGGCTCAT |
| 1463 | ACACTGGAATTGCTAGACCCCGCG | CGCGGGGTCTAGCAATTCCAGTGT |
| 1464 | CTGAGCTGCGTGGGACAACTCCGC | GCGGAGTTGTCCCACGCAGCTCAG |
| 1465 | CAGCTACTAGGGCGCGATGTACCC | GGGTACATCGCGCCCTAGTAGCTG |
| 1466 | ATAATGATGGGACGAGAAGGCCCC | GGGGCCTTCTCGTCCCATCATTAT |
| 1467 | CGACCGAGTGTTACGACATGGTGC | GCACCATGTCGTAACACTCGGTCG |
| 1468 | TGCAGTACCCGCCGCTCCACTAGT | ACTAGTGGAGCGGCGGGTACTGCA |
| 1469 | ATGCTAGCGCGCCTGTCAACGTAC | GTACGTTGACAGGCGCGCTAGCAT |
| 1470 | AGACTCACTGCCGGCTGATCAAAT | ATTTGATCAGCCGGCAGTGAGTCT |
| 1471 | GCCTGGTGCGAAGATAGGGATTCC | GGAATCCCTATCTTCGCACCAGGC |
| 1472 | GGAAAGTTGGCGGATCCGAGCACT | AGTGCTCGGATCCGCCAACTTTCC |
| 1473 | GGCAGTGAGCAATGTGTGACGAGG | CCTCGTCACACATTGCTCACTGCC |
| 1474 | TGAGGTCCTCCCGGCGGACTACGA | TCGTAGTCCGCCGGGAGGACCTCA |
| 1475 | CTCGCCTTAGATCGTGGTTCCGCA | TGCGGAACCACGATCTAAGGCGAG |
| 1476 | GTCGAGGAATATCATCGCAGCCAG | CTGGCTGCGATGATATTCCTCGAC |
| 1477 | GCGAATGCAACGAGACAAGAAGGA | TCCTTCTTGTCTCGTTGCATTCGC |
| 1478 | TTCGCCACCAAGTCGGCATTTGTT | AACAAATGCCGACTTGGTGGCGAA |
| 1479 | CGGTGGCTGACACTTGCCGGATTC | GAATCCGGCAAGTGTCAGCCACCG |
| 1480 | CAAGGAGCAATCAGATGGTCGGAG | CTCCGACCATCTGATTGCTCCTTG |
| 1481 | GTGACCCGGTCCGTTCTAGCTGTG | CACAGCTAGAACGGACCGGGTCAC |
| 1482 | CTCTCGCCCACATAACTGCACAAA | TTTGTGCAGTTATGTGGGCGAGAG |
| 1483 | AAACCTGCCTAAGCAAGCACTGGA | TCCAGTGCTTGCTTAGGCAGGTTT |
| 1484 | TTCCATATTGTACCCCGCGCATGC | GCATGCGCGGGGTACAATATGGAA |
| 1485 | TGCTTGCGATATCACGATACTGCG | CGCAGTATCGTGATATCGCAAGCA |
| 1486 | TTAGTGTTCGAGCCTTGAGCCGGC | GCCGGCTCAAGGCTCGAACACTAA |
| 1487 | CTTGTTGCGCGAGTCCGTCTGGGA | TCCCAGACGGACTCGCGCAACAAG |
| 1488 | GTCAGCTGCCTGCTGGTGCTCTTC | GAAGAGCACCAGCAGGCAGCTGAC |
| 1489 | CATCCCTCGAGGTGTAGGCAACAC | GTGTTGCCTACACCTCGAGGGATG |
| 1490 | CAGATGCACTCCGACGGGATTCAG | CTGAATCCCGTCGGAGTGCATCTG |
| 1491 | CTGAGCCTCGCGAAGCTGTGGCAT | ATGCCACAGCTTCGCGAGGCTCAG |
| 1492 | GCTATGCCACGCCGCAGATAGAGC | GCTCTATCTGCGGCGTGGCATAGC |
| 1493 | AACACCAACCATACCGTCCGTTCA | TGAACGGACGGTATGGTTGGTGTT |
| 1494 | GCCCAGAGCTAAAGCATGTCTGGG | CCCAGACATGCTTTAGCTCTGGGC |
| 1495 | AATGCTGCAATGCTAGCGTCGCTA | TAGCGACGCTAGCATTGCAGCATT |
| 1496 | TCCGGACGCAGTATCCAATCCGGA | TCCGGATTGGATACTGCGTCCGGA |
| 1497 | TAAGACCATGTGGCACCAAGGTGC | GCACCTTGGTGCCACATGGTCTTA |
| 1498 | ACAGCCACACACACGCGCCCACTA | TAGTGGGCGCGTGTGTGTGGCTGT |
| 1499 | TAGAACCGAGCACGGCGCCTTGTA | TACAAGGCGCCGTGCTCGGTTCTA |
| 1500 | TTCGAGTAAGCTGGCAGGACCACT | AGTGGTCCTGCCAGCTTACTCGAA |
| 1501 | CTTTCGCAGGTTCGCAGACAATCC | GGATTGTCTGCGAACCTGCGAAAG |
| 1502 | TACGTCCTGTGCTGTTGACACCGG | CCGGTGTCAACAGCACAGGACGTA |
| 1503 | GTTCGGGTCAATGTTTCGGGGAGA | TCTCCCCGAAACATTGACCCGAAC |
| 1504 | CCCTGTTGTGAAGGGGTTTTGTGA | TCACAAAACCCCTTCACAACAGGG |
| 1505 | GGCAGATTGGTGAACCCCAGATAA | TTATCTGGGGTTCACCAATCTGCC |
| 1506 | CCCTCGGTGTGTTCAAGCCAAATC | GATTTGGCTTGAACACACCGAGGG |
| 1507 | CCCGCGAACATTTGAACAGCTTAA | TTAAGCTGTTCAAATGTTCGCGGG |
| 1508 | CCGTGTCAGTTGCTCCCTGGCACG | CGTGCCAGGGAGCAACTGACACGG |
| 1509 | TCCGTCTCAGCCGCCTCCCTATCC | GGATAGGGAGGCGGCTGAGACGGA |
| 1510 | ATAGCTGGGTCACCACAGGCGGTC | GACCGCCTGTGGTGACCCAGCTAT |
| 1511 | ATAGGCAAGCGGTGTAGCACAGCG | CGCTGTGCTACACCGCTTGCCTAT |
| 1512 | TTAGAAGCCGGTCTGGATTTGCGT | ACGCAAATCCAGACCGGCTTCTAA |
| 1513 | TGCCGACCTTTACCAGGATCCTCG | CGAGGATCCTGGTAAAGGTCGGCA |
| 1514 | GCCCACACTATAACCAAGCTGGCA | TGCCAGCTTGGTTATAGTGTGGGC |
| 1515 | TTGCGCCACTAGTACGGATCTCAA | TTGAGATCCGTACTAGTGGCGCAA |
| 1516 | CTTGCAGTTTATGCTGACCCGTCC | GGACGGGTCAGCATAAACTGCAAG |
| 1517 | TGCCTCCAAATTACTTACCGCCGT | ACGGCGGTAAGTAATTTGGAGGCA |
| 1518 | CCCGTATGCGGAAGCTATGGGCTA | TAGCCCATAGCTTCCGCATACGGG |
| 1519 | TCGTTCAACCCCACACTTCAGTTG | CAACTGAAGTGTGGGGTTGAACGA |
| 1520 | CAATGTGGGGGACATTTCAAGGTT | AACCTTGAAATGTCCCCCACATTG |
| 1521 | TAGCGTCGCACAAATGGCTGACCG | CGGTCAGCCATTTGTGCGACGCTA |
| 1522 | GGTGGCTTCGTGACAATATCGGCC | GGCCGATATTGTCACGAAGCCACC |
| 1523 | CAGCGGCGTCCGAAATTGGCTCTC | GAGAGCCAATTTCGGACGCCGCTG |
| 1524 | GGCTTGCTCTCGTTTTTGATTGCA | TGCAATCAAAAACGAGAGCAAGCC |
| 1525 | ATGCGAGGAGGACACGACCGTTCC | GGAACGGTCGTGTCCTCCTCGCAT |
| 1526 | CCTGTTCACTACGACCCACGGGAA | TTCCCGTGGGTCGTAGTGAACAGG |
| 1527 | GTGCCACGGAGTGCGACTGTTGCT | AGCAACAGTCGCACTCCGTGGCAC |
| 1528 | ACACATCCAAGTCTGACGATGGCC | GGCCATCGTCAGACTTGGATGTGT |
| 1529 | CAGCCCGAAAGGAAAGCCTCCGTG | CACGGAGGCTTTCCTTTCGGGCTG |
| 1530 | AACTGAATGTAGGTGGGCCCCTGT | ACAGGGGCCCACCTACATTCAGTT |
| 1531 | ATTTTCGACGATAAGCTGGCCGGT | ACCGGCCAGCTTATCGTCGAAAAT |
| 1532 | TGAGGGAGAACCCGAAATCTGCTT | AAGCAGATTTCGGGTTCTCCCTCA |
| 1533 | GGCGACTACATCCCCAATTGCTTG | CAAGCAATTGGGGATGTAGTCGCC |
| 1534 | GCAGACGCGGCCTTCCATACTTTT | AAAAGTATGGAAGGCCGCGTCTGC |
| 1535 | ACAACCACATGACGTGTAGCTGCA | TGCAGCTACACGTCATGTGGTTGT |
| 1536 | CTGCTGGGCGCGCAAAGCTTGTTG | CAACAAGCTTTGCGCGCCCAGCAG |
| 1537 | AAGCCTTCTTTGGCTTGCTCCGCT | AGCGGAGCAAGCCAAAGAAGGCTT |
| 1538 | TACCTGCTGCCTGGAGCAAGGCAT | ATGCCTTGCTCCAGGCAGCAGGTA |
| 1539 | GACGCCGCAGCCATGAGTGAGTGT | ACACTCACTCATGGCTGCGGCGTC |
| 1540 | AGTTGGCCGCTTATTTTGCTCACC | GGTGAGCAAAATAAGCGGCCAACT |
| 1541 | CCAGGCGCCTTCGACAGATCCTCA | TGAGGATCTGTCGAAGGCGCCTGG |
| 1542 | GTGTCCCCTCCAGCTAGCCAGTTT | AAACTGGCTAGCTGGAGGGGACAC |
| 1543 | GACAACAAGCCAAGGTGACACGTC | GACGTGTCACCTTGGCTTGTTGTC |
| 1544 | CTACACCGCTCGTGACTCGGCAAA | TTTGCCGAGTCACGAGCGGTGTAG |
| 1545 | TGGTGCCATCAAAGCACGTTGTAC | GTACAACGTGCTTTGATGGCACCA |
| 1546 | ACAATGCGTGTTGCGAAACGCATA | TATGCGTTTCGCAACACGCATTGT |
| 1547 | TTGTCCAGCCATTGTATTTTGCGC | GCGCAAAATACAATGGCTGGACAA |
| 1548 | ACGAGAGATAGCGGACTCCTCCGA | TCGGAGGAGTCCGCTATCTCTCGT |
| 1549 | AGCTTTGTCGTCAGGCGAGCTCTT | AAGAGCTCGCCTGACGACAAAGCT |
| 1550 | GACAGTCGGCGTGCAGTTTGTTGT | ACAACAAACTGCACGCCGACTGTC |
| 1551 | AGCTAGCGACGGCCAACTCACGTA | TACGTGAGTTGGCCGTCGCTAGCT |
| 1552 | CTCCTGTTCGGGGCCGTTACTGGT | ACCAGTAACGGCCCCGAACAGGAG |
| 1553 | ACTGACCGACGCAGTGCCACATAG | CTATGTGGCACTGCGTCGGTCAGT |
| 1554 | AGGTAGGGTCTGGTTTGACTCGCA | TGCGAGTCAAACCAGACCCTACCT |
| 1555 | CCTCCATTTTAGCGCGTTGCCAAT | ATTGGCAACGCGCTAAAATGGAGG |
| 1556 | TTCTTAGGATCCGCGCACTCTTGG | CCAAGAGTGCGCGGATCCTAAGAA |
| 1557 | GTCGAAGGTGTCTACCGTGCGCAG | CTGCGCACGGTAGACACCTTCGAC |
| 1558 | GTCACTCGGCGGCCCAATCACTCG | CGAGTGATTGGGCCGCCGAGTGAC |
| 1559 | TCTCGGTCACCCGTCTTGACCCTT | AAGGGTCAAGACGGGTGACCGAGA |
| 1560 | GCCCTCGACGAACTCATCCTGAAC | GTTCAGGATGAGTTCGTCGAGGGC |
| 1561 | TCCGGCGTACTCTGACACGGCGAT | ATCGCCGTGTCAGAGTACGCCGGA |
| 1562 | AGCCAAATGCTTTCGTGGTTCGGA | TCCGAACCACGAAAGCATTTGGCT |
| 1563 | ACTCCACGCCGCATGTTGCTGTGA | TCACAGCAACATGCGGCGTGGAGT |
| 1564 | GCTTCGAGTCGGTGGCATCTGTAT | ATACAGATGCCACCGACTCGAAGC |
| 1565 | GGTCTTGGGCCATCGACTTGCTGC | GCAGCAAGTCGATGGCCCAAGACC |
| 1566 | GGTATCGGACTGCACTAAGGGCAA | TTGCCCTTAGTGCAGTCCGATACC |
| 1567 | AGCCCATGCGTTCCGGATGATTTG | CAAATCATCCGGAACGCATGGGCT |
| 1568 | GCCAGGGTTAAAAGTGATGGGCTC | GAGCCCATCACTTTTAACCCTGGC |
| 1569 | GACGACGTGCTGGCTACGAAGGGG | CCCCTTCGTAGCCAGCACGTCGTC |
| 1570 | TCCTATTGACCGTGCATCGTGATC | GATCACGATGCACGGTCAATAGGA |
| 1571 | ACCCGCCTCGACTCCACAACTAAA | TTTAGTTGTGGAGTCGAGGCGGGT |
| 1572 | GATGTGGATCACGACCTGCCAGTA | TACTGGCAGGTCGTGATCCACATC |
| 1573 | GTGCCATTGCCACCCATAATGCGT | ACGCATTATGGGTGGCAATGGCAC |
| 1574 | TTAGCCTGTGCACCCAGTCAGGAG | CTCCTGACTGGGTGCACAGGCTAA |
| 1575 | TCCGATGGGAGAGGCTGATCTCAC | GTGAGATCAGCCTCTCCCATCGGA |
| 1576 | CACTACTGAAGTGGCCTGGCGCTG | CAGCGCCAGGCCACTTCAGTAGTG |
| 1577 | TGCGGCCATAGCGATGTGATAGAT | ATCTATCACATCGCTATGGCCGCA |
| 1578 | GATTGCGCTTAACGGAGATGCACG | CGTGCATCTCCGTTAAGCGCAATC |
| 1579 | TCACGTTTGACAACGCCAAGCATT | AATGCTTGGCGTTGTCAAACGTGA |
| 1580 | GCATTGTTTGCTAAAGGCGGCATT | AATGCCGCCTTTAGCAAACAATGC |
| 1581 | AGTCGCTCTACGCGTGCAACGCTG | CAGCGTTGCACGCGTAGAGCGACT |
| 1582 | TAGCTCCATGGAGGTCCGAAAGGG | CCCTTTCGGACCTCCATGGAGCTA |
| 1583 | GACCGGTTGGACCTCACTGGCTTC | GAAGCCAGTGAGGTCCAACCGGTC |
| 1584 | AAGCCGGACAGTCAATGTGCGTAT | ATACGCACATTGACTGTCCGGCTT |
| 1585 | TGCCTCGCTGAGTTCTTCACCGTG | CACGGTGAAGAACTCAGCGAGGCA |
| 1586 | TCGTAGACCTTGCTTTTGGGCTCA | TGAGCCCAAAAGCAAGGTCTACGA |
| 1587 | ACCGCTATGCGCCCTACAAAGCAT | ATGCTTTGTAGGGCGCATAGCGGT |
| 1588 | TAGCGTCACCGTAGCTTGGGGCAG | CTGCCCCAAGCTACGGTGACGCTA |
| 1589 | CTCTCAGCAACTGATGGCACCGGA | TCCGGTGCCATCAGTTGCTGAGAG |
| 1590 | AAAGGAAATGTGGTGCTGGTCGGC | GCCGACCAGCACCACATTTCCTTT |
| 1591 | CCGGCTTAGATGGAGAACAAGTGC | GCACTTGTTCTCCATCTAAGCCGG |
| 1592 | AAGTAAATCGCCTCGCCCAAACCG | CGGTTTGGGCGAGGCGATTTACTT |
| 1593 | TGGGCTGTTCAGCCTACCGGACGT | ACGTCCGGTAGGCTGAACAGCCCA |
| 1594 | GTTTCGGTTCAGCCATGGGCCTAC | GTAGGCCCATGGCTGAACCGAAAC |
| 1595 | GGCCAACATTTCTAGGGGAGTGCC | GGCACTCCCCTAGAAATGTTGGCC |
| 1596 | TTCTTCGTTGGGATTGTCCTCACC | GGTGAGGACAATCCCAACGAAGAA |
| 1597 | TGCACATTGGGGTACGGATCTGAC | GTCAGATCCGTACCCCAATGTGCA |
| 1598 | GGCAGTTAGACGGCAAACTGCAGG | CGTGCAGTTTGCCGTCTAACTGCC |
| 1599 | CGCGTCAGGCTATGAATGGCTCTT | AAGAGCCATTCATAGCCTGACGCG |
| 1600 | GCTGAATGCAAACCTCGGAGCCAT | ATGGCTCCGAGGTTTGCATTCAGC |
| 1601 | CGCTCTGGCGGATTCATTGTTTTC | GAAAACAATGAATCCGCCAGAGCG |
| 1602 | TTTTCAATCAACCCTCCGGACGTA | TACGTCCGGAGGGTTGATTGAAAA |
| 1603 | GTGGTGGAGTCTGAAGCACGACAG | CTGTCGTGCTTCAGACTCCACCAC |
| 1604 | AAACAGGTCCGGATGATGTCTGGA | TCCAGACATCATCCGGACCTGTTT |
| 1605 | GTACCGCGTGTACGCCACCGTTAG | CTAACGGTGGCGTACACGCGGTAC |
| 1606 | TCCAACCTACATTTGCGGAAGGAA | TTCCTTCCGCAAATGTAGGTTGGA |
| 1607 | GACGTACCGTGGTCCCGTGAGTTG | CAACTCACGGGACGACGGTACGTC |
| 1608 | GGCAATCCTACAACCGACGCTGAT | ATCAGCGTCGGTTGTAGGATTGCC |
| 1609 | GGCGGCTGCAGGGTCTACATCGAG | CTCGATGTAGACCCTGCAGCCGCC |
| 1610 | ATACTACGCTGCAGCTGCGCGGGC | GCCCGCGCAGCTGCAGCGTAGTAT |
| 1611 | GGATCGCAATCCCTCCGATGACGA | TCGTCATCGGAGGGATTGCGATCC |
| 1612 | TGGCCTTGCACGGGAGCCGAATCT | AGATTCGGCTCCCGTGCAAGGCCA |
| 1613 | AGGTGCCGACGAAACGACGAATAT | ATATTCGTCGTTTCGTCGGCACCT |
| 1614 | GCTGTTTCACCGTCGTCGTTGTTG | CAACAACGACGACGGTGAAACAGC |
| 1615 | CGGTCCCAATGTTACAACCCAGAC | GTCTGGGTTGTPACATTGGGACCG |
| 1616 | GCAATTCCAGCCACTTTTGACCAA | TTGGTCAAAAGTGGCTGGAATTGC |
| 1617 | ACGGGCGAAAGCTCGGTACGGATA | TATCCGTACCGAGCTTTCGCCCGT |
| 1618 | CGACCCGACTTTTGCTTTCGAGTG | CACTCGAAAGCAAAAGTCGGGTCG |
| 1619 | AATTCAGTGTTTGCGTCATGGTCG | CGACCATGACGCAAACACTGAATT |
| 1620 | CCTGTATGAGGTTCTGGGTCGGCT | AGCCGACCCAGAACCTCATACAGG |
| 1621 | TGGCATACTTGGTGCAAACGCCGT | ACGGCGTTTGCACCAAGTATGCCA |
| 1622 | TCGCCAGTACAGAAACATGCGGGC | GCCCGCATGTTTCTGTACTGGCGA |
| 1623 | CCCGCTGTTGCTCTCATCGTGGAG | CTCCACGATGAGAGCAACAGCGGG |
| 1624 | GCCACAATCTGACCCTGGGAATCA | TGATTCCCAGGGTCAGATTGTGGC |
| 1625 | GCTCAGTCTCGGAAGTTTCGGCTA | TAGCCGAAACTTCCGAGACTGAGC |
| 1626 | CTTCACGGGCCAACGACGGTCGAG | CTCGACCGTCGTTGGCCCGTGAAG |
| 1627 | CGACAGTTCCGTCCGTCTTGAGGA | TCCTCAAGACGGACGGAACTGTCG |
| 1628 | ACGGAGACGCAGTCGAAACGTCCC | GGGACGTTTCGACTGCGTCTCCGT |
| 1629 | CATGCATCCGATTAAGGGGATCAC | GTGATCCCCTTAATCGGATGCATG |
| 1630 | ATTGCGGGAGTCCCTAGCTTTCTG | CAGAAAGCTAGGGACTCCCGCAAT |
| 1631 | GTGTGGAAGATGCAATTGGAACGG | CCGTTCCAATTGCATCTTCCACAC |
| 1632 | ATACAACGGTAGGTGACAGGGGCG | CGCCCCTGTCACCTACCGTTGTAT |
| 1633 | GCCGTGGGAGTAAGGGTACAAAGG | CCTTTGTACCCTTACTCCCACGGC |
| 1634 | GCACGTAGGTGGGCTACTACTCGG | CCGAGTAGTAGCCGACCTACGTGC |
| 1635 | ACTGTGATCTCTTGGGCAAAGGGC | GCCCTTTGCCCAAGAGATCACAGT |
| 1636 | CATGCCTGAACAATCTCGCATCCC | GGGATGCGAGATTGTTCAGGCATG |
| 1637 | GAGCCTGGCTCCACAGCTGTGCTC | GAGCACAGCTGTGGAGCCAGGCTC |
| 1638 | CTTTCGATACCATCGTTGGCGATC | GATCGCCAACGATGGTATCGAAAG |
| 1639 | CCCGGAGGTGAGGCATTGAATATG | CATATTCAATGCCTCACCTCCGGG |
| 1640 | CTCATTCAGCTAAAAGCGGCTGGA | TCCAGCCGCTTTTAGCTGAATGAG |
| 1641 | GAAATGCCCTGGGGACTTTTTGCC | GGCAAAAAGTCCCCAGGGCATTTC |
| 1642 | TTTGCCTTCACAACAGACGCAGCA | TGCTGCGTCTGTTGTGAAGGCAAA |
| 1643 | AAATCCCAAGACGTCGGGGCGTAT | ATACGCCCCGACGTCTTGGGATTT |
| 1644 | CAACGGGCGGTAGCTAAACCGTAA | TTACGGTTTAGCTACCGCCCGTTG |
| 1645 | GGCCAACGACAATGCGAAACCTTC | GAAGGTTTCGCATTGTCGTTGGCC |
| 1646 | GACATCACGCAAAATCTCAGCGCA | TGCGCTGAGATTTTGCGTGATGTC |
| 1647 | ACGTTCCGTCCACAACCGTATGTT | AACATACGGTTGTGGACGGAACGT |
| 1648 | GCTCATAGGTCTTCCGTAGCCCGT | ACGGGCTACGGAAGACCTATGAGC |
| 1649 | GAAACGAGTCTCTCGCGCCCTAGA | TCTAGGGCGCGAGAGACTCGTTTC |
| 1650 | CGGGACAGAAGCAAGTTACATCGG | CCGATGTAACTTGCTTCTGTCCCG |
| 1651 | TGACCGCTCGATACCAGGAGGGTG | CACCCTCCTGGTATCGAGCGGTCA |
| 1652 | CTGGCAATAAAGACCTTCCGACCA | TGGTCGGAAGGTCTTTATTGCCAG |
| 1653 | TGCGCGACGTCATGTTGGTGATTA | TAATCACCAACATGACGTCGCGCA |
| 1654 | GTTGGTTGTGGGAACACACCCGCT | AGCGGGTGTGTTCCCACAACCAAC |
| 1655 | TGTGGGTTCGGAAACACAGGAAGT | ACTTCCTGTGTTTCCGAACCCACA |
| 1656 | GGAAAAAACGGCAATTAGCCGAGT | ACTCGGCTAATTGCCGTTTTTTCC |
| 1657 | TGGTGGGGAGTGCCCTCTATTGGG | CCCAATAGAGGGCACTCCGCACCA |
| 1658 | AACCAACAGGCTGCAGCCCAGACT | AGTCTGGGCTGCAGCCTGTTGGTT |
| 1659 | AAACAGATCCATCTGCACGCCAGG | CCTGGCGTGCAGATGGATCTGTTT |
| 1660 | GGAATACCGCGGCGATTATGGCTT | AAGCCATAATCGCCGCGGTATTCC |
| 1661 | TACTGTTCGCGGCAAACCGTCACT | AGTGACGGTTTGCCGCGAACAGTA |
| 1662 | GATCTCTCGTGGAGCACGTTTTCC | GGAAAACGTGCTCCACGAGAGATC |
| 1663 | GGCATAGCAAACCTTGACCTCCAA | TTGGAGGTCAAGGTTTGCTATGCC |
| 1664 | ATCTGGGATTCGCGAGCCAATATC | GATATTGGCTCGCGAATCCCAGAT |
| 1665 | CGATCAGGATATCATTTACGCCCG | CGGGCGTAAATGATATCCTGATCG |
| 1666 | ACGGTACCGAAACGGTCTCAGCGT | ACGCTGAGACCGTTTCGGTACCGT |
| 1667 | CTCCCATACCTGCGTTCTTACCGA | TCGGTAAGAACGCAGGTATGGGAG |
| 1668 | GCACGAGAACCTAATTGTCGCACA | TGTGCGACAATTAGGTTCTCGTGC |
| 1669 | GCCACACGATCAAGACAGCGCATG | CATGCGCTGTCTTGATCGTGTGGC |
| 1670 | CCCGTTAACTCACGAGCGGTCAAT | ATTGACCGCTCGTGAGTTAACGGG |
| 1671 | AGAGAAGGTCATTGCCTGTCGGTG | CACCGACAGGCAATGACCTTCTCT |
| 1672 | CGGGCCCTCTTAAAGTAGAGCAGG | CCTGCTCTACTTTAAGAGGGCCCG |
| 1673 | ACATCGCGTCCGAGGGAGTTAGCG | CGCTAACTCCCTCGGACGCGATGT |
| 1674 | AATGCCTAATCGAGCCAGCGGATC | GATCCGCTGGCTCGATTAGGCATT |
| 1675 | CTCGATCTTTTTAAACCGGCGCTT | AAGCGCCGGTTTAAAAAGATCGAG |
| 1676 | CGTTCCTGGAAGGCAGGGTCTCAC | GTGAGACCCTGCCTTCCAGGAACG |
| 1677 | CCTGTGCTTACTATCGGCGATCCA | TGGATCGCCGATAGTAAGCACAGG |
| 1678 | GTTAGTCGCCCTATTGGCCTGGTT | AACCAGGCCAATAGGGCGACTAAC |
| 1679 | CCGGTGAGATGACTGTAAATGCCA | TGGCATTTACAGTCATCTCACCGG |
| 1680 | CGTGGTTTAAAACATCGCGCTTCG | CGAAGCGCGATGTTTTAAACCACG |
| 1681 | TAAGACGCAGAAGATGGGGTCCAC | GTGGACCCCATCTTCTGCGTCTTA |
| 1682 | CACCACAGCTTCTTTGTTCGACCC | GGGTCGAACAAAGAAGCTGTGGTG |
| 1683 | TCGGGTCCGTACCACCACTTTTGC | GCAAAAGTGGTGGTACGGACCCGA |
| 1684 | CCAAGCCCCGAGTACCGAAGATTT | AAATCTTCGGTACTCGGGGCTTGG |
| 1685 | TCCGTGATATGGTCGTGGCGCGGT | ACCGCGCCACGACCATATCACGGA |
| 1686 | TGTCTGTGTCATGGCACCTCGCAT | ATGCGAGGTGCCATGACACAGACA |
| 1687 | AGGACTGCACTGTGCACGTCTGAT | ATCAGACGTGCACAGTGCAGTCCT |
| 1688 | CCATCCTCATGTACAGCGCCGCTG | CAGCGGCGCTGTACATGAGGATGG |
| 1689 | GTACCCGCGCCTTCCTCGACACAG | CTGTGTCGAGGAAGGCGCGGGTAC |
| 1690 | ACGGGTCCTGGTCGACTAAGGCTT | AAGCCTTAGTCGACCAGGACCCGT |
| 1691 | CGTATCGAAGGCGTGTACAACCGG | CCGGTTGTACACGCCTTCGATACG |
| 1692 | TGCCCGCCCTTTATGCAACGCTCA | TGAGCGTTGCATAAAGGGCGGGCA |
| 1693 | AAACTTACGAGACGGCGGCTGCCA | TGGCAGCCGCCGTCTCGTAAGTTT |
| 1694 | AAGTCTGACAAACGGAACGGGTGT | ACACCCGTTCCGTTTGTCAGACTT |
| 1695 | TAAGCGCAGACCAAAGTATGCGGC | GCCGCATACTTTGGTCTGCGCTTA |
| 1696 | GCAGTTTTTCAGATCCTCCGCAAA | TTTGCGGAGGATCTGAAAAACTGC |
| 1697 | TCGGAAGCATTTACGCGATCTCAG | CTGAGATCGCGTAAATGCTTCCGA |
| 1698 | CACAGAAACGGTTGAACGAACGCC | GGCGTTCGTTCAACCGTTTCTGTG |
| 1699 | GCATGCTCAGATGGTCGTGCTCAC | GTGAGCACGACCATCTGAGCATGC |
| 1700 | AAGGATTCTCGCTTCCGGCATGAT | ATCATGCCGGAAGCGAGAATCCTT |
| 1701 | GGTGGGGTAGCGCTGGTATGAAAA | TTTTCATACCAGCGCTACCCCACC |
| 1702 | ATTATTACGGGACCGAACCAACGG | CCGTTGGTTCGGTCCCGTAATAAT |
| 1703 | GCGCGAGTGTCATGATGTTCACGT | ACGTGAACATCATGACACTCGCGC |
| 1704 | GACATTCGTGACTTGGTCGTCCGC | GCGGACGACCAAGTCACGAATGTC |
| 1705 | TCATTAGTGCAGGCACCGATCAAG | CTTGATCGGTGCCTGCACTAATGA |
| 1706 | GAGTTGTGCGGAGTCATCGGAGTC | GACTCCGATGACTCCGCACAACTC |
| 1707 | GCCTTTACAGATTTGGCGGGCTAT | ATAGCCCGCCAAATCTGTAAAGGC |
| 1708 | ATGGCGTTTGCGAAGTCGATACAG | CTGTATCGACTTCGCAAACGCCAT |
| 1709 | TGCATCGGCCTCAATCAGAGAACT | AGTTCTCTGATTGAGGCCGATGCA |
| 1710 | ACAATCATGGCAATCTGGCAAATG | CATTTGCCAGATTGCCATGATTGT |
| 1711 | GACGTGGAAGAGTGCAGATCAGCA | TGCTGATCTGCACTCTTCCACGTC |
| 1712 | AGGGCAGGGGACGGACAGTAAGTC | GACTTACTGTCCGTCCCCTGCCCT |
| 1713 | GCATAGGGCGAATCTAGTACGGGC | GCCCGTACTAGATTCGCCCTATGC |
| 1714 | TCCGGCGCATCCTCATTAGCAACT | AGTTGCTAATGAGGATGCGCCGGA |
| 1715 | TGGCCGCTTCCACTAATATTGGAC | GTCCAATATTAGTGGAAGCGGCCA |
| 1716 | CCGGCGGACGGCTCTTGTCAATGA | TCATTGACAAGAGCCGTCCGCCGG |
| 1717 | CGAGCAACCCAAAAGGAAGCAGTA | TACTGCTTCCTTTTGGGTTGCTCG |
| 1718 | GCGTATGATTCGGCAATCCGCCAG | CTGGCGGATTGCCGAATCATACGC |
| 1719 | AGTACCGCTACAACGCTGGTTCGC | GCGAACCAGCGTTGTAGCGGTACT |
| 1720 | GGGCAGGCCAGGTCCACCTGAGAA | TTCTCAGGTGGACCTGGCCTGCCC |
| 1721 | CCACTTCTGTGACCGAACCGTGCT | AGCACGGTTCGGTCACAGAAGTGG |
| 1722 | CCTGGTACCAGGCAGCAGTTGATT | AATCAACTGCTGCCTGGTACCAGG |
| 1723 | TTAGGGTACCGTCGAGAGACGCCA | TGGCGTCTCTCGACGGTACCCTAA |
| 1724 | GGTTGCTTGTGCGCGTGAGGTAGT | ACTACCTCACGCGCACAAGCAACC |
| 1725 | TGCTTCGACCGATGAAACTCGAAG | CTTCGAGTTTCATCGGTCGAAGCA |
| 1726 | TGCCACCCATACTATGCCCAGTGG | CCACTGGGCATAGTATGGGTGGCA |
| 1727 | TGTGCGGCAACGCGTGAAGACGTT | AACGTCTTCACGCGTTGCCGCACA |
| 1728 | TGAGAGAAGCTGGCCTCGGATCAG | CTGATCCGAGGCCAGCTTCTCTCA |
| 1729 | TATTGCGAATTCGAGTACGTGCCC | GGGCACGTACTCGAATTCGCAATA |
| 1730 | CGAGAGGGGTTCCCCAGTGATCGA | TCGATCACTGGGGAACCCCTCTCG |
| 1731 | TGCCTGGGGTGTCGTTCTAATTCT | AGAATTAGAACGACACCGCAGGCA |
| 1732 | GTGCGTCATTGTGGGTCATCCCAA | TTGGGATGACCCACAATGACGCAC |
| 1733 | AGGGCTCCCAGCATACCAACGTTG | CAACGTTGGTATGCTGGGAGCCCT |
| 1734 | AACTAGCCGCACCTTTGTGCAGAG | CTCTGCACAAAGGTGCGGCTAGTT |
| 1735 | TTAGCCCAGCCCTTCAATGGGAAC | GTTCCCATTGAAGGGCTGGGCTAA |
| 1736 | CGGCCTCGGTTGTACGGGTAGTCT | AGACTACCCGTACAACCGAGGCCG |
| 1737 | TCTTTGAGGCGCGGACCCGCATAT | ATATGCGGGTCCGCGCCTCAAAGA |
| 1738 | GATGGTTCGCCCTTGTGTCGCAGC | GCTGCGACACAAGGGCGAACCATC |
| 1739 | GAGATTCAATACAGGCCGCGGGTC | GACCCGCGGCCTGTATTGAATCTC |
| 1740 | AGGGCGAAGGAAGGTTCCGTTTTT | AAAAACGGAACCTTCCTTCGCCCT |
| 1741 | CTCGACCCCTGCCACTACTGGTTC | GAACCAGTAGTGGCAGGGGTCGAG |
| 1742 | TGTTCCGCGGTCTACGCATTACTG | CAGTAATGCGTAGACCGCGGAACA |
| 1743 | GAGACGACGTCCTACACCCGCTAA | TTAGCGGGTGTAGGACGTCGTCTC |
| 1744 | AGATTGCGACAGCGACACGTGATT | AATCACGTGTCGCTGTCGCAATCT |
| 1745 | GATACCGTTGGGCATTTCTCGGTA | TACCGAGAAATGCCCAACGGTATC |
| 1746 | GATTGGGAGGCATTCAGCGACGGA | TCCGTCGCTGAATGCCTCCCAATC |
| 1747 | AGGAGGAAACGAGGGCGTAGGTTC | GAACCTACGCCCTCGTTTCCTCCT |
| 1748 | GCCAAACAACGTCTGACGCCTAGC | GCTAGGCGTCAGACGTTGTTTGGC |
| 1749 | TTTAATGCGGAAAGGATGCACGCG | CGCGTGCATCCTTTCCGCATTAAA |
| 1750 | TTATCGGCCGTTAAAATGGGATGG | CCATCCCATTTTAACGGCCGATAA |
| 1751 | CCTTGGATTCGTTCATCGCTAGCA | TGCTAGCGATGAACGAATCCAAGG |
| 1752 | AAGTGAACGTGCAGTGGTCTTCGA | TCGAAGACCACTGCACGTTCACTT |
| 1753 | TCCTTACCCCTCGTTCAAACGCCT | AGGCGTTTGAACGAGGGGTAAGGA |
| 1754 | ATTCCTGAACCATGCATGGCCTGT | ACAGGCCATGCATGGTTCAGGAAT |
| 1755 | AGCGAGACGCTCGATCACGAACTA | TAGTTCGTGATCGAGCGTCTCGCT |
| 1756 | GCTGGTCTGGCTCGCTGTTTAGAA | TTCTAAACAGCGAGCCAGACCAGC |
| 1757 | CGTGCGCGGCATAAAGATAGGTCT | AGACCTATCTTTATGCCGCGCACG |
| 1758 | TCTGGCACTCACATCGGACAGTCT | AGACTGTCCGATGTGAGTGCCAGA |
| 1759 | ACCATTGGAGGACCACAGAGCTCC | GGAGCTCTGTGGTCCTCCAATGGT |
| 1760 | TCCAGGGTCGGAGTACATGGCGGG | CCCGCCATGTACTCCGACCCTGGA |
| 1761 | ATATGCCGTCGGATCGTACACGCA | TGCGTGTACGATCCGACGGCATAT |
| 1762 | TGCTGGCGTCAACACTTCCCGATT | AATCGGGAAGTGTTGACGCCAGCA |
| 1763 | CAGGGCGGTGCGGTGAACTAGCCA | TGGCTAGTTCACCGCACCGCCCTG |
| 1764 | CATGGACTGCCGTACATCAGCTGG | CCAGCTGATGTACGGCAGTCCATG |
| 1765 | CCGGCCATACGCTGGCAAGATTAC | GTAATCTTGCCAGCGTATGGCCGG |
| 1766 | AGCGGACACCTGTACTCTCCTCCA | TGGAGGAGAGTACAGGTGTCCGCT |
| 1767 | GGAGCCACACCAGTCGAAGATGGT | ACCATCTTCGACTGGTGTGGCTCC |
| 1768 | CGCCACCGGAAATTGAAAAGACTG | CAGTCTTTTCAATTTCCGGTGGCG |
| 1769 | TGAAAGGGATGTTGCTTCTTGACG | CGTCAAGAAGCAACATCCGTTTCA |
| 1770 | TTGAAGCGGTGAAGAGCCTGTCCT | AGGACAGGCTCTTCACCGCTTCAA |
| 1771 | CGAACCAAGCTGCATTGTCAGTGG | CCACTGACAATGCAGCTTGGTTCG |
| 1772 | GAGTCTGCGCTTGCAATCTTTGCG | CGCAAAGATTGCAAGCGCAGACTC |
| 1773 | GCTGGGTATAGTTGCCTGGCAATG | CATTGCCAGGCAACTATACCCAGC |
| 1774 | GCAGGCGTTCCATATTCGCAACCC | GGGTTGCGAATATGGAACGCCTGC |
| 1775 | GCGCCAACTAATACCTCCACCGCG | CGCGGTGGAGGTATTAGTTGGCGC |
| 1776 | TGGCGTTCAGTGCAACGCTGGTTA | TAACCAGCGTTGCACTGAACGCCA |
| 1777 | CAAAACTGACGGGTATGGGAGCGC | GCGCTCCCATACCCGTCAGTTTTG |
| 1778 | AGGTGTCGCTGGAACCCGACTTGT | ACAAGTCGGGTTCCAGCGACACCT |
| 1779 | CTTCCAAAAGCGCAATTGGCTTTG | CAAAGCCAATTGCGCTTTTGGAAG |
| 1780 | TCGGGCTTCTCGCAATTCTGTCAG | CTGACAGAATTGCGAGAAGCCCGA |
| 1781 | GCCAAAAGAATGCGCTGGGTAGGT | ACCTACCCAGCGCATTCTTTTGGC |
| 1782 | TGGTGCCCGCACCGAGAGACTGTA | TACAGTCTCTCGGTGCGGGCACCA |
| 1783 | CGAGGCCGTAGTGGGGACTGCTCT | AGAGCAGTGCCCACTACGGCCTCG |
| 1784 | CGATCTGCGCATAGAGGGGACTTT | AAAGTCCCCTCTATGCGCAGATCG |
| 1785 | TGTGCAATCGGCCTTCTCAGAGCC | GGCTCTGAGAAGGCCGATTGCACA |
| 1786 | GATCACCTGGACCGCTACCGTTTT | AAAACGGTAGCGGTCCAGGTGATC |
| 1787 | ATGGGGAGTTAAGGACCCTGCACC | GGTGCAGGGTCCTTAACTCCCCAT |
| 1788 | CATTGTGGACAGCCAATGGTGGCT | AGCCACCATTGGCTGTCCACAATG |
| 1789 | CCATCACCATGCCACGGTAAGATC | GATCTTACCGTGGCATGGTGATGG |
| 1790 | GCACCCGTGTCGTTGGTTAGCAAG | CTTGCTAACCAACGACACGGGTGC |
| 1791 | GGAGTGGGTTCCGCGAATTCACTG | CAGTGAATTCGCGGAACCCACTCC |
| 1792 | GGGGATTTCCTTTCGCAGGCTCGA | TCGAGCCTGCGAAAGGAAATCCCC |
| 1793 | CATTGATCATGTGCACTTGCACCA | TGGTGCAAGTGCACATGATCAATG |
| 1794 | AGCAGCGCTGCGCTTGTTTCGGAT | ATCCGAAACAAGCGCAGCGCTGCT |
| 1795 | CGAGTAACGCGGTTGCTTTGCGAA | TTCGCAAAGCAACCGCGTTACTCG |
| 1796 | TGGCCTGGAACATAGGTGGAACTC | GAGTTCCACCTATGTTCCAGGCCA |
| 1797 | CGCACACCAAGCGTTTATTGAGAA | TTCTCAATAAACGCTTGGTGTGCG |
| 1798 | TCACCTTCACAGTGGGCATACAGC | GCTGTATGCCCACTGTGAAGGTGA |
| 1799 | CAAATATCCCTGAGCCCTCGAGCT | AGCTCGAGGGCTCAGGGATATTTG |
| 1800 | GGGAGCTGGTGAGCAGATGTAACG | CGTTACATCTGCTCACCAGCTCCC |
| 1801 | AGGATTGCTTTTGCGTTATGCGGA | TCCGCATAACGCAAAAGCAATCCT |
| 1802 | ATCGTTTGGGCGCTACGCAATTGT | ACAATTGCGTAGCGCCCAAACGAT |
| 1803 | CCGATTTGTCCCAAATGGAACGTT | AACGTTGCATTTGGGACAAATCGG |
| 1804 | AAGGGTCAAGCTCATGGAGCGGAA | TTCCGCTCCATGAGCTTGACCCTT |
| 1805 | TCTGACGTCGTTCAAGGGCTCGCT | AGCGAGCCCTTGAACGACGTCAGA |
| 1806 | CGCACCACTCCGAGGTATTTGTCT | AGACAAATACCTCGGAGTGGTGCG |
| 1807 | AAGGGGTGAAAAAGGAGAAGCCGA | TCGGCTTCTCCTTTTTCACCCCTT |
| 1808 | AAACCACGCAAATGGCGATACCAT | ATGGTATCGCCATTTGCGTGGTTT |
| 1809 | CAGAAGGGATGACGCCTTAAGTCG | CGACTTAAGGCGTCATCCCTTCTG |
| 1810 | CATGACGAGAGCGGACCTGAAGTG | CAGTTCAGGTCCGCTCTCGTCATG |
| 1811 | CTGGACATGTTTGTTTCGCCACTG | CAGTGGCGAAACAAACATGTCCAG |
| 1812 | AAGACCGACTCTCGTCGTTTGCAC | GTGCAAACGACGAGAGTCGGTCTT |
| 1813 | GCGCGATTACATACCGTTTCCGTA | TACGGAAACGGTATGTAATCGCGC |
| 1814 | CACTGACCGGACCCAACCTAACAT | ATGTTAGGTTGGGTCCGGTCAGTG |
| 1815 | AGTGCAAGTCTAGACACGCCCGAG | CTCGGGCGTGTCTAGACTTGCACT |
| 1816 | GGTTGGTGCGAGATCCTGGACTGT | ACAGTCCAGGATCTCGCACCAACC |
| 1817 | GGTCGTCCCGAAACGTAAACGAGG | CCTCGTTTACGTTTCGGGACGACC |
| 1818 | GACTAGTACGATCACGGGGCGGGT | ACCCGCCCCGTGATCGTACTAGTC |
| 1819 | CCGACCTGACCCTGTGTACAGGTT | AACCTGTACAGAGGGTCAGGTCGG |
| 1820 | TGCTCACTGCCCACACTGTTATGG | CCATAACAGTGTGGGCAGTGAGCA |
| 1821 | CGAGGAAACACATTTCTTCGGGCC | GGCCCGAAGAAATGTGTTTCCTCG |
| 1822 | TGGCACCGGGTGGATTCTTGTCTA | TAGACAAGAATCCACCCGGTGCCA |
| 1823 | GAGGCACGGTGATAGTGGTTGTGC | GCACAACCACTATCACCGTGCCTC |
| 1824 | ATGCAGATGGATCTTTTTCGACGC | GCGTCGAAAAAGATCCATCTGCAT |
| 1825 | TGCGATAGCCAAAGAGTCGAGGAC | GTCCTCGACTCTTTGGCTATCGCA |
| 1826 | ATGGCGTGTCAGCGAACTGCCTGG | CCAGGCAGTTCGCTGACACGCCAT |
| 1827 | CAATGCAGCTCGGAAGTCAGGTCG | CGACCTGACTTCCGAGCTGCATTG |
| 1828 | AGGATCAGTGCACATGTCCCCTCA | TGAGGGGACATGTGCACTGATCCT |
| 1829 | CACATCTTGGCTGTCACCCGAGAA | TTCTCGGGTGACAGCCAAGATGTG |
| 1830 | CGCATTATCACCTCAATGCCAGTG | CACTGGCATTGAGGTGATAATGCG |
| 1831 | ACATCCGCAGACTCCCTATAGCCC | GGGCTATAGGGAGTCTGCGGATGT |
| 1832 | GTGAACCCGAACGAGGGGAGTCTC | GAGACTCCCCTCGTTCGGGTTCAC |
| 1833 | GCGTAGGGAATTTGCCTCACGACT | AGTCGTGAGGCAAATTCCCTACGC |
| 1834 | TTTACGCGTCGCTCGGTTGTAGTG | CACTACAACCGAGCGACGCGTAAA |
| 1835 | GAGAGGCGTCTAGGCGGTTCTAGC | GCTAGAACCGCCTAGACGCCTCTC |
| 1836 | GCATGCTGATAACGAATGCTTCCC | GGGAAGCATTCGTTATCAGCATGC |
| 1837 | CTGAAGCTCGTGTGCGATGAGGGA | TCCCTCATCGCACACGAGCTTCAG |
| 1838 | ACAACGGCATGAGGAGGCTTTTTC | GAAAAAGCCTCCTCATGCCGTTGT |
| 1839 | TTTGGAGACGCCAGTACGCGTGGT | ACCACGCGTACTGGCGTCTCCAAA |
| 1840 | GCTATCATTTGGTGTAAGCCCGCC | GGCGGGCTTACACCAAATGATAGC |
| 1841 | TCAACATCCAGGGCGGTGCTTGGT | ACCAAGCACCGCCCTGGATGTTGA |
| 1842 | TTCGATGTAATCCCCAAAGATGCC | GGCATCTTTGGGGATTACATCGAA |
| 1843 | GGACCTTCGGCAGGTTATCGCCGT | ACGGCGATAACCTGCCGAAGGTCC |
| 1844 | AGTAAGAAGAGGCAGGCCCCACCT | AGGTGGGGCCTGCCTCTTCTTACT |
| 1845 | AACGGCTCCCCGTCGTACTGCTTA | TAAGCAGTACGACGGGGAGCCGTT |
| 1846 | CCTATACCGTCGTGGTTCCACGTT | AACGTGGAACCACGACGGTATAGG |
| 1847 | CCGCGCAGGCGCTAATACTCAAGG | CCTTGAGTATTAGCGCCTGCGCGG |
| 1848 | AAATGGGCCAGTGAAATCCTTGGT | ACCAAGGATTTCACTGGCCCATTT |
| 1849 | ACGGTTTCGAATACTGCTGGGCAG | CTGCCCAGCAGTATTCGAAACCGT |
| 1850 | CCGCTTGAGGTTCAGGTGAGAGCT | AGCTCTGACCTGAACCTCAAGCGG |
| 1851 | ATCGTGCCCGAAGACACTTAAACG | CGTTTAAGTGTCTTCGGGCACGAT |
| 1852 | ACCTGAACCAGGGCGATTGCTTTA | TAAAGCAATCGCCCTGGTTCAGGT |
| 1853 | ACCCTATACGCTGGGCTAAGCGGG | CCCGCTTAGCCCAGCGTATAGGGT |
| 1854 | TGTTTCGCGACTAGAAGCCTTTGC | GCAAAGGCTTCTAGTCGCGAAACA |
| 1855 | GAAGTTGGCGGCTCACCCGTATTA | TAATACGGGTGAGCCGCCAACTTC |
| 1856 | TGGCTACACCGCTTAGGAGGAACC | GGTTCCTCCTAAGCGGTGTAGCCA |
| 1857 | CCACAGTTGCGTGACTTACATCGC | GCGATGTAAGTCACGCAACTGTGG |
| 1858 | ACTGCCACTGCGTCTGAAGAGTGG | CCACTCTTCAGACGCAGTGGCAGT |
| 1859 | GCGCCAGCAAATTTCGTGTGGTGT | ACACCACACGAAATTTGCTGGCGC |
| 1860 | TGCCTCCGTCGAGCCGAATAGCCA | TGGCTATTCGGCTCGACGGAGGCA |
| 1861 | GTACAAACGGGCGCTATTTCGTCC | GGACGAAATAGCGCCCGTTTGTAC |
| 1862 | GCTTCCCTGGCTCTGAACGGAAAC | GTTTCCGTTCAGAGCCAGGGAAGC |
| 1863 | CGGCTACCCAGGCAGATAAGCTGA | TCAGCTTATCTGCCTGGGTAGCCG |
| 1864 | GGTTGGACCCGACAGGGAATTTCC | GGAAATTCCCTGTCGGGTCCAACC |
| 1865 | GGGGAATACCCGGCGTTTGTAATA | TATTACAAACGCCGGGTATTCCCC |
| 1866 | TGGTTCGGTGAGGTTATGTTCGGT | ACCGAACATAACCTCACCGAACCA |
| 1867 | TCGGTAGGGTTCAGTCGCTGAGGA | TCCTCAGCGACTGAACCCTACCGA |
| 1868 | TTCGGAGTGTGCCGGTGCTAGTAC | GTACTAGCACCGGCACACTCCGAA |
| 1869 | TCGTACTGGAATGATGGCCGGGCC | GGCCCGGCCATCATTCCAGTACGA |
| 1870 | TCCGTCGACCGTCCAGCGAAGTTT | AAACTTCGCTGGACGGTCGACGGA |
| 1871 | AGGGAATATAACAACACCGCGCAC | GTGCGCGGTGTTGTTATATTCCCT |
| 1872 | ATGTCCCGGAAACCAGCTACCTCA | TGAGGTAGCTGGTTTCCGGGACAT |
| 1873 | ACCAGCGACTTAGATAGCCGTCCG | CGGACGGCTATCTAAGTCGCTGGT |
| 1874 | GGAAAACCTCCTTTGCGTCAACCA | TGGTTGACGCAAAGGAGGTTTTCC |
| 1875 | ACGTGCGTGCATACCCAAGAGGAC | GTCCTCTTGGGTATGCACGCACGT |
| 1876 | ACGCCACTTTCCCTAGAACCAACG | CGTTGGTTCTAGGGAAAGTGGCGT |
| 1877 | CGAAGTACGCAATAGTGCCACCCT | AGGGTGGCACTATTGCGTACTTCG |
| 1878 | GATCCCGGCGGATCACCTATCAAT | ATTGATAGGTGATCCGCCGGGATC |
| 1879 | AGAAAGCGACCGTTTCAGGCTAGC | GCTAGCCTGAAACGGTCGCTTTCT |
| 1880 | CGCTCCCTTTCATAGTCCTCTCCG | CGGAGAGGACTATGAAAGGGAGCG |
| 1881 | GTGGGTGGTCATAACGACAGCAGA | TCTGCTGTCGTTATGACCACCCAC |
| 1882 | CTGGAGGCTGCATCGTTCGTAACA | TGTTACGAACGATGCAGCCTCCAG |
| 1883 | CACCATGAGTTTCGGAGCGAGGAT | ATCCTCGCTCCGAAACTCATGGTG |
| 1884 | CAAGCTGCGTTCGATGAGAGATTG | CAATCTCTCATCGAACGCAGCTTG |
| 1885 | CCTGGGAGCAATGACCGCTCTGGT | ACCAGAGCGGTCATTGCTCCCAGG |
| 1886 | TCCGGCGCTCTACCAAGATGAGAC | GTCTCATCTTGGTAGAGCGCCGGA |
| 1887 | CGACCGCGTCGCGTATACTATCCG | CGGATAGTATACGCGACGCGGTCG |
| 1888 | AACATTCGCTAGTGGGGTCCAACA | TGTTGGACCCCACTAGCGAATGTT |
| 1889 | TGTATGATCATCCGACCGAGCAGC | GCTGCTCGGTCGGATGATCATACA |
| 1890 | AGTGCGCCGAGAGGGTGAATAGAC | GTCTATTCACCCTCTCGGCGCACT |
| 1891 | AGGCTTGTTCTGGACCAGCACCAT | ATGGTGCTGGTCCAGAACAAGCCT |
| 1892 | GGGGCCACATAAAGAATTCCGAAC | GTTCGGAATTCTTTATGTGGCCCC |
| 1893 | TGGTGAAGATAAATCCGCATGGCA | TGCCATGCGGATTTATCTTCACCA |
| 1894 | ATTTCCACCACGCTCTTGCCAAAT | ATTTGGCAAGAGCGTGGTGGAAAT |
| 1895 | CGCGTAAAGCTGTCACCGATGACC | GGTCATCGGTGACAGCTTTACGCG |
| 1896 | TCCCCAACCGGTAACAACAGCGAC | GTCGCTGTTGTTACCGGTTGGGGA |
| 1897 | CCTCTGCTCGCCTTACACCCATGG | CCATGGGTGTAAGGCGAGCAGAGG |
| 1898 | CAAGCTGCTCCTGTGCTGAAGGGC | GCCCTTCAGCACAGGAGCAGCTTG |
| 1899 | AAACGAACGATGGTCGGTAGACCG | CGGTCTACCGACCATCGTTCGTTT |
| 1900 | TCAGTTCGATGGCTATTGCGCCTC | GAGGCGCAATAGCCATCGAACTGA |
| 1901 | GGCTCTCAACGGACGCAAATCATA | TATGATTTGCGTCCGTTGAGAGCC |
| 1902 | AGTAGAGTGTTGCGGCTGCCGATC | GATCGGCAGCCGCAACACTCTACT |
| 1903 | AGACACTAGACCGCCGTGACCTGA | TCAGGTCACGGCGGTCTAGTGTCT |
| 1904 | ACCGAGCACCGAATTTCCTTGTCC | GGACAAGGAAATTCGGTGCTCGGT |
| 1905 | CCGTGGCCAAGATACGAACGAATT | AATTCGTTCGTATCTTGGCCACGG |
| 1906 | CCTCCTACAGCATCCACATGAGGG | CCCTCATGTGGATGCTGTAGGAGG |
| 1907 | CACTCGGCAAATACGTATGCGCAT | ATGCGCATACGTATTTGCCGAGTG |
| 1908 | ACCGAGTTGAAGCACGAATTTGGG | CCCAAATTCGTGCTTCAACTCGGT |
| 1909 | GACCACCTCGGAAGATCGTTCTGC | GCAGAACGATCTTCCGAGGTGGTC |
| 1910 | TCAACTGGGCAAACGAAGAGCACA | TGTGCTCTTCGTTTGCCCAGTTGA |
| 1911 | GCTTAGCCTCACACGTGCATACCA | TGGTATGCACGTGTGAGGCTAAGC |
| 1912 | CTGCGGTCTCCAAGTACCATTTCG | CGAAATGGTACTTGGAGACCGCAG |
| 1913 | GTTCCGTATTACGGCGGCCATAAG | CTTATGGCCGCCGTAATACGGAAC |
| 1914 | ATCGACGCAACCGGATAGTCTCTG | CAGAGACTATCCGGTTGCGTCGAT |
| 1915 | CGCAGATAAACCGGCATCTTTCAG | CTGAAAGATGCCGGTTTATCTGCG |
| 1916 | ACCTGCCAATACGGGTCTACGGTT | AACCGTAGACCCGTATTGGCAGGT |
| 1917 | ACACCTGTTGCCATGCTGATCCGT | ACGGATCAGCATGGCAACAGGTGT |
| 1918 | AAACTGTCTACTGCGCAATTCCGC | GCGGAATTGCGCAGTAGACAGTTT |
| 1919 | GCAACTAGCCCGTGCTAGGATCGT | ACGATCCTAGCACGGGCTAGTTGC |
| 1920 | TCGTAGTGGTGGATTGTTGTGCGT | ACGCACAACAATCCACCACTACGA |
| 1921 | GGCTTACTCCTCAATTGCGACACG | CGTGTCGCAATTGAGGAGTAAGCC |
| 1922 | CACGACTCCCTGCCAGATTTGATT | AATCAAATCTGGCAGGGAGTCGTG |
| 1923 | CTTAGACGTCGGCAATGTCACGTC | GACGTGACATTGCCGACGTCTAAG |
| 1924 | CTCAGAGCACAATCTGCCCTGCCT | AGGCAGGGCAGATTGTGCTCTGAG |
| 1925 | GCTAGGAAAGTCGGCATTCATGGG | CCCATGAATGCCGACTTTCCTAGC |
| 1926 | AAAGCCCCAAAATTCCGCCTAACC | GGTTAGGCGGAATTTTGGGGCTTT |
| 1927 | GCGCAACGCTAAGGGACTATCAAG | CTTGATAGTCCCTTAGCGTTGCGC |
| 1928 | CGTCCGCTGGGATGAGTCTCCTGC | GCAGGAGACTCATCCCAGCGGACG |
| 1929 | ACAGGCCTCGTGATTGGTGTGGGT | ACCCACACCAATCACGAGGCCTGT |
| 1930 | CATTCTCCTTCCGGGACCACGCCT | AGGCGTGGTCCCGGAAGGAGAATG |
| 1931 | TCGGAGTTGACCAAGCTCAGTGCG | CGCACTGAGCTTGGTCAACTCCGA |
| 1932 | ACGCGCCACTGCAATTGCAAACAC | GTGTTTGCAATTGCAGTGGCGCGT |
| 1933 | AGTTCATGGAGCCGGCGTATTGTT | AACAATACGCCGGCTCCATGAACT |
| 1934 | ACGTTTAATGCGGGGCCCGCCTAC | GTAGGCGGGCCCCGCATTAAACGT |
| 1935 | TGAGGCTTTAGCCTACGCGCAGGT | ACCTGCGCGTAGGCTAAAGCCTCA |
| 1936 | CAGCGTTATGAGCGCGGAGTTTAT | ATAAACTCCGCGCTCATAACGCTG |
| 1937 | GTCCACGTGACCACGGATAGTTGG | CCAACTATCCGTGGTCACGTGGAC |
| 1938 | GATTATGCTCCTACGCCTGCTCCG | CGGAGCAGGCGTAGGAGCATAATC |
| 1939 | TCGTCAAGGGCATGATGTGTGGGA | TCCCACACATCATGCCCTTGACGA |
| 1940 | GATGGACCGCCAAAGACACCTTGA | TCAAGGTGTCTTTGGCGGTCCATC |
| 1941 | TACACGAGGATGGGGTCAAGCTTT | AAAGCTTGACCCCATCCTCGTGTA |
| 1942 | ACACGCACAAAACGTTTGAAAGGC | GCCTTTCAAACGTTTTGTGCGTGT |
| 1943 | GTTATCGTGGGCCGATGGTACTGA | TCAGTACCATCGGCCCACGATAAC |
| 1944 | ACATGACCGTATCCGCCTGCTTCG | CGAAGCAGGCGGATACGGTCATGT |
| 1945 | GAAGGCGAACCACTGAAACTACGC | GCGTAGTTTCAGTGGTTCGCCTTC |
| 1946 | TGACTTTTGCAACGGGTGGAACCA | TGGTTCCACCCGTTGCAAAAGTCA |
| 1947 | TGAATTCGTAGGTTTTGGGTGCGG | CCGCACCCAAAACCTACGAATTCA |
| 1948 | AGCATTTATGAAGCGGCCATTGCG | CGCAATGGCCGCTTCATAAATGCT |
| 1949 | TGCTCCTCGCGTTGGTACCGTGAG | CTCACGGTACCAACGCGAGGAGCA |
| 1950 | CGCAGCAAGAAACAGCAACTGTTG | CAACAGTTGCTGTTTCTTGCTGCG |
| 1951 | AGACGCTTGGAGTGAAAACTCGGA | TCCGAGTTTTCACTCCAAGCGTCT |
| 1952 | CATTCGTAGAATGCCCCAAATGGA | TCCATTTGGGGCATTCTACGAATG |
| 1953 | CCAGAAGGTTCGGGACCCGTCGTG | CACGACGGGTCCCGAACCTTCTGG |
| 1954 | GAGAAGCCGGTTCTCAGAGCACAT | ATGTGCTCTGAGAACCGGCTTCTC |
| 1955 | TTGCGTTGCAAGATATCTGGCCCG | CGGGCCAGATATCTTGCAACGCAA |
| 1956 | GGGTTGCATGTTCAGGCAAGACGA | TCGTCTTGCCTGAACATGCAACCC |
| 1957 | CTCACGAAGGTGACATATCACGCC | GGCGTGATATGTCACCTTCGTGAG |
| 1958 | GCCCGAGATACGGGTTCAAAAAGA | TCTTTTTGAACCCGTATCTCGGGC |
| 1959 | CATCTTCGCGCTTCTTCACTCCGC | GCGGAGTGAAGAAGCGCGAAGATG |
| 1960 | TTACACGGTAAGCGTACGGCCGCC | GGCGGCCGTACGCTTACCGTGTAA |
| 1961 | ACCTTCGGACAATGTGGCGTTCGC | GCGAACGCCACATTGTCCGAAGGT |
| 1962 | TGAATGGTTCTGCTAGGCCCACAC | GTGTGGGCCTAGCAGAACCATTCA |
| 1963 | CACGCCTGTCTGACATATGGATGC | GCATCCATATGTCAGACAGGCGTG |
| 1964 | CGCCTCAACCCAATCTGAGAACGT | ACGTTCTCAGATTGGGTTGAGGCG |
| 1965 | TTACGCTTACTGCGAGCTGGGTCC | GGACCCAGCTCGCAGTAAGCGTAA |
| 1966 | GGCTTGTGGGGCAATACGCATCTT | AAGATGCGTATTGCCCCACAAGCC |
| 1967 | CACTCTCCTTTGGATGCGGAACAA | TTGTTCCGCATCCAAAGGAGAGTG |
| 1968 | GACCAGCCATCACGTAACGGCCCT | AGGGCCGTTACGTGATGGCTGGTC |
| 1969 | AGGAACCGGATGTGGTTATGGAGC | GCTCCATAACCACATCCGGTTCCT |
| 1970 | ATCCATGGGCAACTGAGCCTATGC | GCATAGGCTCAGTTGCCCATGGAT |
| 1971 | GGAACAGCACTTGTTACCGCCCAC | GTGGGCGGTAACAAGTGCTGTTCC |
| 1972 | TGGCTCGCTTCAAGCCTGTTTGCT | AGCAAACAGGCTTGAAGCGAGCCA |
| 1973 | CAAACGTGAGGTCATGACCACCAT | ATGGTGGTCATGACCTCACGTTTG |
| 1974 | ACCGATGTCTTGAAGTCCGGAGGT | ACCTCCGGACTTCAAGACATCGGT |
| 1975 | CGAAAATGCATGATGATCTCCCCT | AGGGGAGATCATCATGCATTTTCG |
| 1976 | TTTGGTATTCTCGCTGCACCGTTG | CAACGGTGCAGCGAGAATACCAAA |
| 1977 | GCGTACTCAACCACATTCCCGACC | GGTCGGGAATGTGGTTGAGTACGC |
| 1978 | AGCAAACAACAGCGGTCCGAGCAT | ATGCTCGGACCGCTGTTGTTTGCT |
| 1979 | GGACTAGGAGCGGGGATAGCTGAG | CTCAGCTATCCCCGCTCCTAGTCC |
| 1980 | CCTTAACGAAAACCTGTCGACCGC | GCGGTCGACAGGTTTTCGTTAAGG |
| 1981 | CTCGATCGCATAAGCAAGAAACCG | CGGTTTCTTGCTTATGCGATCGAG |
| 1982 | CCCGTTGTTTGGGCGACAAAAAGT | ACTTTTTGTCGCCCAAACAACGGG |
| 1983 | CGGCGGCTCTCGCATGATCTCGTT | AACGAGATCATGCGAGAGCCGCCG |
| 1984 | CGGATGGAGAGGAGTCTACGTCCC | GGGACGTAGACTCCTCTCCATCCG |
| 1985 | CAGAACAATATCGTGCGTCAACCG | CGGTTGACGCACGATATTGTTCTG |
| 1986 | CCTTTGCGCGCTCCGAGTAAGGTA | TACCTTACTCGGAGCGCGCAAAGG |
| 1987 | GGAAACGGCACCTATCTGTCGTGA | TCACGACAGATAGGTGCCGTTTCC |
| 1988 | CGACCGACAAAACCAAATGCCGCC | GGCGGCATTTGGTTTTGTCGGTCG |
| 1989 | CCAAGGGTGTGGGAGCTGAAGAGA | TCTCTTCAGCTCCCACACCCTTGG |
| 1990 | TTAAGTGCGCATAGTCCTCGTGGG | CCCACGAGGACTATGCGCACTTAA |
| 1991 | GCCTGGTGGGGTAAGTCATGATGC | GCATCATGACTTACCCCACCAGGC |
| 1992 | GAGCAGCAGATTGATGCGCTTATG | CATAAGCGCATCAATCTGCTGCTC |
| 1993 | TGCGCCAACTTCCGGAATATTTGC | GCAAATATTCCGGAAGTTGGCGCA |
| 1994 | AACCCCATCATGAAATGCTCTCCG | CGGAGAGCATTTCATGATGGGGTT |
| 1995 | GTCCAACGGTACTGGCGTGATGTT | AACATCACGCCAGTACCGTTGGAC |
| 1996 | ACTCGGCTGATCGTGAGATGGTGA | TCACCATCTCACGATCAGCCGAGT |
| 1997 | ATTCGTGGGCGCATCTCGGTATGT | ACATTCCGAGATGCGCCCACGAAT |
| 1998 | TCCCGTCCTGTAATCCAGGGAACA | TGTTCCCTGGATTACAGGACGGGA |
| 1999 | CTTCGCTGCACCTACATTGCGCCA | TGGCGCAATGTAGGTGCAGCGAAG |
| 2000 | GCGTGTAGATGACTGTGCTTTGGG | CCCAAAGCACAGTCATCTACACGC |
| 2001 | CTATGGTATCGAGACATCGGCGGA | TCCGCCGATGTCTCGATACCATAG |
| 2002 | CCTCGTACTCCGTCGTATGCACAA | TTGTGCATACGACGGAGTACGAGG |
| 2003 | TGGTGCGTCCGTAGTGCCTGCACT | AGTGCAGGCACTACGGACGCACCA |
| 2004 | CGCGATCCTAGTTGAAAGCTTTGC | GCAAAGCTTTCAACTAGGATCGCG |
| 2005 | ACGATCCAGGTGTTGGGCACTAAG | CTTAGTGCCCAACACCTGGATCGT |
| 2006 | CCAATCTAGGATACACCACGCCCG | CGGGCGTGGTGTATCCTAGATTGG |
| 2007 | GATACGTGGGGTATAGGCGGGCCC | GGGCCCGCCTATACCCCACGTATC |
| 2008 | CATGGAACAAACCGTCGTAGGGGA | TCCCCTACGACGGTTTGTTCCATG |
| 2009 | ACACTCGCGCAGTATTCGAGTCGT | ACGACTCGAATACTGCGCGAGTGT |
| 2010 | CTCAGTCTCGAAGGTGATCCGACC | GGTCGGATCACCTTCGAGACTGAG |
| 2011 | TCCCAATCCCCGTGGTATCGTCGT | ACGACGATACCACGGGGATTGGGA |
| 2012 | AATCAACGTAGTTCCGGTGGTCCG | CGGACCACCGGAACTACGTTGATT |
| 2013 | CTTAACAACCCAGGGGTTTGGGCT | AGCCCAAACCCCTGGGTTGTTAAG |
| 2014 | CTACCGCTGCATGGCGTTAGATTG | CAATCTAACGCCATGCAGCGGTAG |
| 2015 | TTATTGGTGGCGGACGGAGTGAGT | ACTCACTCCGTCCGCCACCAATAA |
| 2016 | TTAAGGGTGAACTCAACCGCGTGA | TCACGCGGTTGAGTTCACCCTTAA |
| 2017 | TTTGATTGAAACGCTGCGCACTAC | GTAGTGCGCAGCGTTTCAATCAAA |
| 2018 | TCATGTGTAGGTCGCGGCCGTCAC | GTGACGGCCGCGACCTACACATGA |
| 2019 | CTCCGAACCTTCTGGGCCTCTTTT | AAAAGAGGCCCAGAAGGTTCGGAG |
| 2020 | CTGTTGCCCATTGGCCCGACACTC | GAGTGTCGGGCCAATGGGCAACAG |
| 2021 | CACGATCGCTGAGCAACACATCAC | GTGATGTGTTGCTCAGCGATCGTG |
| 2022 | CGGATCATAAGCGTCCGCCTTCGT | ACGAAGGCGGACGCTTATGATCCG |
| 2023 | AGGTTAACGCAACATGTGATCCGC | GCGGATCACATGTTGCGTTAACCT |
| 2024 | GGGAAAAACAGCTAAGCCTTGCGA | TCGCAAGGCTTAGCTGTTTTTCCC |
| 2025 | ACTTATTGCCGGGATCCGTACACA | TGTGTACGGATCCCGGCAATAAGT |
| 2026 | TGCGGTCTGGAAAGGAAGGGAGGG | CCCTCCCTTCCTTTCCAGACCGCA |
| 2027 | GCTGCCACCTGGACATCGCATACA | TGTATGCGATGTCCAGGTGGCAGC |
| 2028 | GCAGGCATGACAGTGGCGTAGTAC | GTACTACGCCACTGTCATGCCTGC |
| 2029 | GCGGCCCTGATGGTTTGGCTGAGC | GCTCAGCCAAACCATCAGGGCCGC |
| 2030 | TCCCCATTTAGTCCCCTCCATCAC | GTGATGGAGGGGACTAAATGGGGA |
| 2031 | GCAACACAAATGCGAGCGTAGGAG | CTCCTACGCTCGCATTTGTGTTGC |
| 2032 | GGCGTTTGTATTCGAGCCACGTAG | CTACGTGGCTCGAATACAAACGCC |
| 2033 | GGTAACGTCGCACGTGGAATTCCG | CGGAATTCCACGTGCGACGTTACC |
| 2034 | ACTTCACAACGCTCCGTTGGACAC | GTGTCCAACGGAGCGTTGTGAAGT |
| 2035 | CCGAATTATAAAGCGCAAGGCACA | TGTGCCTTGCGCTTTATAATTCGG |
| 2036 | GGACCCGATAAGACTCTGACGCCG | CGGCGTCAGAGTCTTATCGGGTCC |
| 2037 | ACCCGTTTCTCGTAGGAACCTGCT | AGCAGGTTCCTACGAGAAACGGGT |
| 2038 | CACGTTCGACTGTATCTGGTTGCC | GGCAACCAGATACAGTCGAACGTG |
| 2039 | CCTCGGATGGGCCCATGACCTTGA | TCAAGGTCATGGGCCCATCCGAGG |
| 2040 | GGACGCCTGCTGTAGGGGTTTGAT | ATCAAACCCCTACAGCAGGCGTCC |
| 2041 | CTCGAGCGTGGGCTAAAAGAGCAT | ATGCTCTTTTAGCCCACGCTCGAG |
| 2042 | TTTACTTCTTAGGGCGCGTTTGGG | CCCAAACGCGCCCTAAGAAGTAAA |
| 2043 | ACCACCAACATAGCGCGCACTAGT | ACTAGTGCGCGCTATGTTGGTGGT |
| 2044 | TGGTTACACGGCAGCCCGCGTAAG | CTTACGCGGGCTGCCGTGTAACCA |
| 2045 | TTATGGTACGTTGCTGCGTGCGGG | CCCGCACGCAGCAACGTACCATAA |
| 2046 | ACCGCGGATCTAACGAATCCCATT | AATGGGATTCGTTAGATCCGCGGT |
| 2047 | CATGATCCCGCCCTTAGGTTAAGC | GCTTAACCTAAGGGCGGGATCATG |
| 2048 | TACCGCTTCAAAGGGTTGCCGAAT | ATTCGGCAACCCTTTGAAGCGGTA |
| 2049 | GCACCGCGTCAATATTACCGAGGA | TCCTCGGTAATATTGACGCGGTGC |
| 2050 | GTGTCGCGGCTTTACAGAAGGAGA | TCTCCTTCTGTAAAGCCGCGACAC |
| 2051 | GCAAGCCATACCGCAATAAACTCG | CGAGTTTATTGCGGTATGGCTTGC |
| 2052 | ATGAGGTCGTGCTGCGTTCACGAG | CTCGTGAACGCAGCACGACCTCAT |
| 2053 | CGAGACTAGTGCCGATGCAGGGTA | TACCCTGCATCGGCACTAGTCTCG |
| 2054 | GCCTCATCATAGACGCTGGATGCA | TGCATCCAGCGTCTATGATGAGGC |
| 2055 | GACAGGCGTCGGTAAGCTCTCAAG | CTTGAGAGCTTACCGACGCCTGTC |
| 2056 | GCTACGAATCTTCCCTGTCGCCAC | GTGGCGACAGGGAAGATTCGTAGC |
| 2057 | TTTGGCAGAACGTACCAGTGGGGT | ACCCCACTGGTACGTTCTGCCAAA |
| 2058 | GGACAATAAGCACCGGAGAATGCG | CGCATTCTCCGGTGCTTATTGTCC |
| 2059 | TCATGAACCTTCTGATGCCGCGAA | TTCGCGGCATCAGAAGGTTCATGA |
| 2060 | CGCCGCATTACCTTAAAAACGTGC | GCACGTTTTTAAGGTAATGCGGCG |
| 2061 | ACGAGTCCAACCGCCTCATTGATT | AATCAATGAGGCGGTTGGACTCGT |
| 2062 | GCGAAGAGTTGCTACTCTTCCGCC | GGCGGAAGAGTAGCAACTCTTCGC |
| 2063 | CGTCGGCAACAATCTTTTTCGTGA | TCACGAAAAAGATTGTTGCCGACG |
| 2064 | AATCCTGTGCACCCGTGAGACGCG | CGCGTCTCACGGGTGCACAGGATT |
| 2065 | AACCTATATGCATCAACGCGAGCC | GGCTCGCGTTGATGCATATAGGTT |
| 2066 | GAACTTGGCAAAACAGCCCGGAAA | TTTCCGGGCTGTTTTGCCAAGTTC |
| 2067 | CTCTATGGCCGTTTGCCGTCTGCA | TGCAGACGGCAAACGGCCATAGAG |
| 2068 | AGTGCACCGGGTTGTGGACACAAT | ATTGTGTCCACAACCCGGTGCACT |
| 2069 | CCTGGCTTTTCACACGCCAAGAAA | TTTCTTGGCGTGTGAAAAGCCAGG |
| 2070 | CACTCAGCGTAGCCTGAAGCCTGG | CCAGGCTTCAGGCTACGCTGAGTG |
| 2071 | GAATTATCGACCGCAGCGGTGTCG | CGACACCGCTGCGGTCGATAATTC |
| 2072 | GTGACATCACATGGTGGCCGAGCG | CGCTCGGCCACCATGTGATGTCAC |
| 2073 | AGCACCTTGCCGAGTCACCAGTGA | TCACTGGTGACTCGGCAAGGTGCT |
| 2074 | TAGGTTGCAGGAATGGTGGGCACC | GGTGCCCACCATTCCTGCAACCTA |
| 2075 | GTCCCATACGTGTGGTACGCGGAT | ATCCGCGTACCACACGTATGGGAC |
| 2076 | TCGGATACTCTCGCGTGCCACGGG | CCCGTGGCACGCGAGAGTATCCGA |
| 2077 | CAACGTTCGCCCCTAAGCCCAAAT | ATTTGGGCTTAGGGGCGAACGTTG |
| 2078 | GTTAGGTCACCGCGGCATATCCTA | TAGGATATGCCGCGGTGACCTAAC |
| 2079 | GTTCACCGGCCTCTACTTGGGTTT | AAACCCAAGTAGAGGCCGGTGAAC |
| 2080 | AATCCGCGTCTAGGTCATGTGGTC | GACCACATGACCTAGACGCGGATT |
| 2081 | GCTACGCCTCTGGAGGTGGTACCC | GGGTACCACCTCCAGAGGCGTAGC |
| 2082 | CAGGGAATGCTACAAAGGGTCCAA | TTGGACCCTTTGTAGCATTCCCTG |
| 2083 | AAGGGTTAGCTGCCCGGTTAACAG | CTGTTAACCGGGCAGCTAACCCTT |
| 2084 | CCTCGCAAGCGCGATATTTATGCC | GGCATAAATATCGCGCTTGCGAGG |
| 2085 | GCCTCCCGGTCATGGTCAAGGGAA | TTCCCTTGACCATGACCGGGAGGC |
| 2086 | GCTGTTGAGCGGCGACCTGTGCAC | GTGCACAGGTCGCCGCTCAACAGC |
| 2087 | CGCTGACTTAGCTCTGATGTGCCG | CGGCACATCAGAGCTAAGTCAGCG |
| 2088 | TTCATGGCATTCATCACGAAGGAA | TTCCTTCGTGATGAATGCCATGAA |
| 2089 | TAGTGTTATGCCCGCGTGTGAATG | CATTCACACGCGGGCATAACACTA |
| 2090 | CATGTAAGGGCACGGTCGTGGGCA | TGCCCACGACCGTGCCCTTACATG |
| 2091 | CAGGAAGCTCGCTCCGTGATGCAC | GTGCATCACGGAGCGAGCTTCCTG |
| 2092 | CCTGCTGATAGCAACCTCACTGCA | TGCAGTGAGGTTGCTATCAGCAGG |
| 2093 | ACTACGAGGGGCAGGGTCTAGGCG | CGCCTAGACCCTGCCCCTCGTAGT |
| 2094 | CATAATGTGGGTGCTGACGCCGAT | ATCGGCGTCAGCACCCACATTATG |
| 2095 | TAGCGAATCCACACAGAGCCGCTC | GAGCGGCTCTGTGTGGATTCGCTA |
| 2096 | TCGCGAAATCCCTAAATGCTGTGC | GCACAGGATTTAGGGATTTCGCGA |
| 2097 | TGGCACGAATCAAGCCACCAACTC | GAGTTGGTGGCTTGATTCGTGCCA |
| 2098 | GCGGACCGTCTTTGCTATCTGACG | CGTCAGATAGCAAAGACGGTCCGC |
| 2099 | AGGCCCCGCCTTGTAATTGGTCAT | ATGACCAATTACAAGGCGGGGCCT |
| 2100 | CTGGTCCCATACGCCGCTGACTAG | CTAGTCAGCGGCGTATGGGACCAG |
| 2101 | TGCTAACTGCGGCCCTACAGAGTC | GACTCTGTAGGGCCGCAGTTAGCA |
| 2102 | TGGTTTTATGTTCGGTAGCGTCCG | CGGACGCTACCGAACATAAAACCA |
| 2103 | AGCTCAAACTTCTCCCACGGGATG | CATCCCGTGGGAGAAGTTTGAGCT |
| 2104 | CGCGAAGATAGTGAAATCCGCATC | GATGCGGATTTCACTATCTTCGCG |
| 2105 | GAGTGAAACCTCTCGCGGGTTGCA | TGCAACCCGCGAGAGGTTTCACTC |
| 2106 | TCGAATGCTCTGCAGTGACGTCAA | TTGACGTCACTGCAGAGCATTCGA |
| 2107 | AGGTGGCAATGATCGACGACCCTG | CAGGGTCGTCGATCATTGCCACCT |
| 2108 | GTCCGGAGCCGTGCAAAGCAATAA | TTATTGCTTTGCACGGCTCCGGAC |
| 2109 | CTTTTGGGGATTAGAGGCCGACAA | TTGTCGGCCTCTAATCCCCAAAAG |
| 2110 | GGCATAAAGGCTTCCGTTCCTGTC | GACAGGAACGGAAGCCTTTATGCC |
| 2111 | GCGGACCGTAAAGCGGGCAGATAG | CTATCTGCCCGCTTTACGGTCCGC |
| 2112 | TTTCAAGAGTGCATCGAATCCACG | CGTGGATTCGATGCACTCTTGAAA |
| 2113 | CCGGCATCCCTTCTCGCTGTTGCC | GGCAACAGCGAGAAGGGATGCCGG |
| 2114 | ACACAGAGACGCGAACGGAGTGCA | TGCACTCCGTTCGCGTCTCTGTGT |
| 2115 | AGCGGCATTCTCCCACTCGTTACT | AGTAACGAGTGGGAGAATGCCGCT |
| 2116 | GGAGCGTACTGCGCCTCGCAAGTC | GACTTGCGAGGCGCAGTACGCTCC |
| 2117 | AAACCCGAATGACACGGCAGATAA | TTATCTGCCGTGTCATTCGGGTTT |
| 2118 | AACCAGCGGATCGATAAAACGACA | TGTCGTTTTATCGATCCGCTGGTT |
| 2119 | GGTGTCCACCCGTTAACGCCGGTA | TACCGGCGTTAACGGGTGGACACC |
| 2120 | AGCGCGACGTGGCTTGCCGTTAAA | TTTAACGGCAAGCCACGTCGCGCT |
| 2121 | TCCCACGGCTATAGGTCCAACGAC | GTCGTTGGACCTATAGCCGTGGGA |
| 2122 | ATCAACGAACGATGCCGTTAGGTG | CACCTAACGGCATCGTTCGTTGAT |
| 2123 | GAGGCTAAGCCGTATGGCCGAGGC | GCCTCGGCCATACGGCTTAGCCTC |
| 2124 | ACGGTCCGAAATGGTTAGAGGCAC | GTGCCTCTAACCATTTCGGACCGT |
| 2125 | ACGCAAACCATTCCTCGAGTAGGC | GCCTACTCGAGGAATGGTTTGCGT |
| 2126 | TTACACGCTCGCTATTGGGCCATA | TATGGCCCAATAGCGAGCGTGTAA |
| 2127 | CTCGGCACGGGTTTAGAACGCCGG | CCGGCGTTCTAAACCCGTGCCGAG |
| 2128 | ATTCGGTAAGGTATCGGGCTAGCG | CGCTAGCCCGATACCTTACCGAAT |
| 2129 | AGCACACCGTTATACATGACGGCG | CGCCGTCATGTATAACGGTGTGCT |
| 2130 | AGTCCCTGCCGTTCGCTCATGGAA | TTCCATGAGCGAACGGCAGGGACT |
| 2131 | GGGCTTATGACCAGTCAGGTTGGA | TCCAACCTGACTGGTCATAAGCCC |
| 2132 | GGTCACCACACGAGTGCCTGGTCT | AGACCAGGCACTCGTGTGGTGACC |
| 2133 | TTGATCGTGTCTCCCGAAACCCTC | GAGGGTTTCGGGAGACACGATCAA |
| 2134 | ATTGTCGCGATCGGCATTTCTTAA | TTAAGAAATGCCGATCGCGACAAT |
| 2135 | GGGTCCAACGACTTCTCGCTGCTG | CAGCAGCGAGAAGTCGTTGGACCC |
| 2136 | CAAATTCCTTGGGGGCCATAGTGG | CCACTATGGCCCCCAAGGAATTTG |
| 2137 | CCAGAGTATCCGCCGTTAGACGGT | ACCGTCTAACGGCGGATACTCTGG |
| 2138 | TCCTGCAGATCATCTCGTGTCTGG | CCAGACACGAGATGATCTGCAGGA |
| 2139 | TGCGGGAGATTTGAACAAGCTGTA | TACAGCTTGTTCAAATCTCCCGCA |
| 2140 | TTAGACGCCGAGCTAGGCAACGTC | GACGTTGCCTAGCTCGGCGTCTAA |
| 2141 | TTTCGGCAGAATCTCCGATTCAAC | GTTGAATCGGAGATTCTGCCGAAA |
| 2142 | TGGCGAGCAGACCTACAAGACAGA | TCTGTCTTGTAGGTCTGCTCGCCA |
| 2143 | GGCGACAGACCGGTACATCGGCCA | TGGCCGATGTACCGGTCTGTCGCC |
| 2144 | TCTAGACCTGCGTTTCGTGGGACC | GGTCCCACGAAACGCAGGTCTAGA |
| 2145 | GCCGAGCGTGGTACCATACGTTCA | TGAACGTATGGTACCACGCTCGGC |
| 2146 | TAATCACACCCGCTTTCTGTGGCT | AGCCACAGAAAGCGGGTGTGATTA |
| 2147 | GGCCGGAGCCATTGGACACTTCTT | AAGAAGTGTCCAATGGCTCCGGCC |
| 2148 | CCTGTAGACCTGCATGGATCGCTG | CAGCGATCCATGCAGGTCTACAGG |
| 2149 | ATCGCCGTTCCCGCAAAATAAGCA | TGCTTATTTTGCGGGAACGGCGAT |
| 2150 | TGGATCAACGGGGTAGTGAAAACG | CGTTTTCACTACCCCGTTGATCCA |
| 2151 | AAGCGACGATGCTTTCTTGAGCTG | CAGCTCAAGAAAGCATCGTCGCTT |
| 2152 | CACGGGCACGTGTTCTACGCTTGC | GCAAGCGTAGAACACGTGCCCGTG |
| 2153 | ACGGGCTGGGACAAGAGCTAGAAA | TTTCTAGCTCTTGTCCCAGCCCGT |
| 2154 | GGTAACTGGCTCCGCTCTCACATC | GATGTGAGAGCGGAGCCAGTTACC |
| 2155 | ACTCTGGCTGTTGGCGAACGTGAC | GTCACGTTCGCCAACAGCCAGAGT |
| 2156 | GACCGAGGACCAGTCCTTGCTCTC | GAGAGCAAGGACTGGTCCTCGGTC |
| 2157 | AGTAGCTCTTGCGGCCTAACGGCA | TGCCGTTAGGCCGCAAGAGCTACT |
| 2158 | TTCTTGTCCTGGGGGAGAGCAGTG | CACTGCTCTCCCCCAGGACAAGAA |
| 2159 | TTAGCAGGGAGGTTGTCGGCTCAT | ATGAGCCGACAACCTCCCTGCTAA |
| 2160 | AGAACGTGGATTGTACGCTCCGCC | GGCGGAGCGTACAATCCACGTTCT |
| 2161 | CTTCACAGCCTGGAGCCACCAATG | CATTGGTGGCTCCAGGCTGTGAAG |
| 2162 | GAGATCGATGAAACGCACCAGCGG | CCGCTGGTGCGTTTCATCGATCTC |
| 2163 | GGGTCCAGAGTTGGTGTGGGATAA | TTATCCCACACCAACTCTGGACCC |
| 2164 | CCGTCCACCCCAGATAGGAATCAC | GTGATTCCTATCTGGGGTGGACGG |
| 2165 | TGCCTCGCTTCTGTGAATCTACGA | TCGTAGATTCACAGAAGCGAGGCA |
| 2166 | GATCACAGCGTCCGCGCATAACGG | CCGTTATGCGCGGACGCTGTGATC |
| 2167 | ATGACGCCTTACATGACGCACCTT | AAGGTGCGTCATGTAAGGCGTCAT |
| 2168 | GCGTGGAATAACGCCCTTAGTTCA | TGAACTAAGGGCGTTATTCCACGC |
| 2169 | GGTCTACCATTTCTCGCCCGACCG | CGGTCGGGCGAGAAATGGTAGACC |
| 2170 | ACACCTCTCTGGCGTAGACGCTCA | TGAGCGTCTACGCCAGAGAGGTGT |
| 2171 | GTAGAGGTGCTCAGGACTCGTCGC | GCGACGAGTCCTGAGCACCTCTAC |
| 2172 | GTAAGCAGGAGGCGAAGGCGCGAA | TTCGCGCCTTCGCCTCCTGCTTAC |
| 2173 | TCTAAGGGCCGTTTCAATCGACCT | AGGTCGATTGAAACGGCCCTTAGA |
| 2174 | AACCTGATTTCAGGGTCAGCCCGA | TCGGGCTGACCCTGAAATCAGGTT |
| 2175 | GTCACGCGATTGGCCCACCTATTA | TAATAGGTGGGCCAATCGCGTGAC |
| 2176 | ACGATGCCGCGCATGTAACCTAGT | ACTAGGTTACATGCGCGGCATCGT |
| 2177 | TGAGAGATGTCTCGTCAACGCCTG | CAGGCGTTGACGAGACATCTCTCA |
| 2178 | GCATATCTCGCGGTGACAGACGAA | TTCGTCTGTCACCGCGAGATATGC |
| 2179 | GACCCAACGTCGAAATTGTGCGAT | ATCGCACAATTTCGACGTTGGGTC |
| 2180 | TGAAAATCGGGGCATCTAGTTTGG | CCAAACTAGATGCCCCGATTTTCA |
| 2181 | CCGCGAAAAGGATTTGTGTACGCA | TGCGTACACAAATCCTTTTCGCGG |
| 2182 | CATTCCATTTATCCGCAGTTCGCT | AGCGAACTGCGGATAAATGGAATG |
| 2183 | CCTGTCTGTCGAGCCAGCGTCTAT | ATAGACGCTGGCTCGACAGACAGG |
| 2184 | TCAGCGCGGCTAAACAAGTTATGC | GCATAACTTGTTTAGCCGCGCTGA |
| 2185 | ACGCCTACGAACGACCCAAGAGAG | CTCTCTTGGGTCGTTCGTAGGCGT |
| 2186 | TGCGCATCTACCATTGTGTGGATC | GATCCAGACAATGGTAGATGCGCA |
| 2187 | AAGTCCGCGCTCGCTCCTGTAATA | TATTACAGGAGCGAGCGCGGACTT |
| 2188 | GCTGGGTCATTGCTCGAGTAACCA | TGGTTACTCGAGCAATGACCCAGC |
| 2189 | TGGAGCGTTCTGGCAATGACCGAC | GTCGGTCATTGCCAGAACGCTCCA |
| 2190 | CAAGTCAATTCTTGGCCAATTCGG | CCGAATTGGCCAAGAATTGACTTG |
| 2191 | CGTTCATGCAAGGATCCCAGGTTA | TAACCTGGGATCCTTGCATGAACG |
| 2192 | ATGCCAATAGAAGCTGGGGATGCT | AGCATCCCCAGCTTCTATTGGCAT |
| 2193 | CCTAACTCTCCCTTGAGGCCGTTC | GAACGGCCTCAAGGGAGAGTTAGG |
| 2194 | ATCTCGGCGAAGGTTCCAAACATT | AATGTTTGGAACCTTCGCCGAGAT |
| 2195 | GCGACAGATTACGCTGCGGTTTTC | GAAAACCGCAGCGTAATCTGTCGC |
| 2196 | AAGCCCAGACGGCCAACACGTTAC | GTAACGTGTTGGCCGTCTGGGCTT |
| 2197 | TCAAGTTCAAATCACATCCCGTGG | CCACGGGATGTGATTTGAACTTGA |
| 2198 | GATTGTCGTTCTGTCTGTGAGGCG | CGCCTCACAGACAGAACGACAATC |
| 2199 | ACCGAACTATGTTCCGGCATGGCA | TGCCATGCCGGAACATAGTTCGGT |
| 2200 | CGTCATCGGGTGTGCAATGCCGTT | AACGGCATTGCACACCCGATGACG |
| 2201 | CGGACGGAGTCACGTTTGTGCACT | AGTGCACAAACGTGACTCCGTCCG |
| 2202 | TAAACAAGTCGTGTGCCTTTGCCG | CGGCAAAGGCACACGACTTGTTTA |
| 2203 | TAATTACTGGCCTGTGGAGCAGGC | GCCTGCTCCACAGGCCAGTAATTA |
| 2204 | GGAGCGGCCCGAATGGTGCTCTTA | TAAGAGCACCATTCGGGCCGCTCC |
| 2205 | ACTAAGCAAGGCTTGGATGTGCGT | ACGCACATCCAAGCCTTGCTTAGT |
| 2206 | GGCAGCTCAGCGGCAGTACGCTAC | GTAGCGTACTGCCGCTGAGCTGCC |
| 2207 | GCGAGGCGAATTATCCGCGGATTT | AAATCCGCGGATAATTCGCCTCGC |
| 2208 | CATACGACACACCTTGGGGTGCTA | TAGCACCCCAAGGTGTGTCGTATG |
| 2209 | TGCTTGGGCTTTAAACCCCGTTTT | AAAACGGGGTTTAAAGCCCAAGCA |
| 2210 | CCGGTTGGAAAACGCAAATATCGG | CCGATATTTGCGTTTTCCAACCGG |
| 2211 | AAACTAGCTAGCCGCACCCGCAAG | CTTGCGGGTGCGGCTAGCTAGTTT |
| 2212 | GTTGTTCCACCAGTGATCACGCAG | CTGCGTGATCACTGGTGGAACAAC |
| 2213 | GCCGCTGACAAGATGATCATCGTT | AACGATGATCATCTTGTCAGCGGC |
| 2214 | CTTTCATAAAGCCAACCGATGCCC | GGGCATCGGTTGGCTTTATGAAAG |
| 2215 | CTGACTGCATCTCGAAAGCGGGTG | CACCCGCTTTCGAGATGCAGTCAG |
| 2216 | ATTTCTTCGGAGAATCGGCCACGT | ACGTGGCCGATTCTCCGAAGAAAT |
| 2217 | CATTTCGGGCCCTAGCTACTGCGC | GCGCAGTAGCTAGGGCCCGAAATG |
| 2218 | CCGATCCCGCACATCCGTATCCTG | CAGGATACGGATGTGCGGGATCGG |
| 2219 | TATCACCGGGAGCGTCTTATCGTG | CACGATAAGACGCTCCCGGTGATA |
| 2220 | TAGGGCTCGTGCACCGATTAGAGG | CCTCTAATCGGTGCACGAGCCCTA |
| 2221 | GCGTGGCACTCGCTTGTCTAGGTA | TACCTAGACAAGCGAGTGCCACGC |
| 2222 | CTCAACGAACTCAAGGGCCGCTAC | GTAGCGGCCCTTGAGTTCGTTGAG |
| 2223 | AGCCTGGTATCGACCAATCCTGCA | TGCAGGATTGGTCGATACCAGGCT |
| 2224 | TACGCGTTCTAGTTGGCCGGATCC | GGATCCGGCCAACTAGAACGCGTA |
| 2225 | TTTATGGGTTTGTGCCTGATGGGT | ACCCATCAGGCACAAACCCATAAA |
| 2226 | GGGACCCCTAGCAACGTCACCTTA | TAAGGTGACGTTGCTAGGGGTCCC |
| 2227 | CTGCCTCCCCAGGAGTCATTGGAT | ATCCAATGACTCCTGGGGAGGCAG |
| 2228 | AACCCCGCAAGACCAGTACCAATC | GATTGGTACTGGTCTTGCGGGGTT |
| 2229 | GGTCACATACGCGCTAAAAAGCGC | GCGCTTTTTAGCGCGTATGTGACC |
| 2230 | AAATGGCTCCGACCAGTTAGGGAC | GTCCCTAACTGGTCGGAGCCATTT |
| 2231 | AACGCGGCACGCTTAAAGGTGCAT | ATGCACCTTTAAGCGTGCCGCGTT |
| 2232 | GATCGCACGCCGATTAACCTTACA | TGTAAGGTTAATCGGCGTGCGATC |
| 2233 | CCTCCTGATTGGGAGTGCGGAATT | AATTCCGCACTCCCAATCAGGAGG |
| 2234 | CGGAGGGTAATAGGCTCCTCTGCG | CGCAGAGGAGCCTATTACCCTCCG |
| 2235 | ACAAGAACTGGACATTACCGCGGG | CCCGCGGTAATGTCCAGTTCTTGT |
| 2236 | TGTCGTCTTAAAGGCCTTTGTGCG | CGCACAAAGGCCTTTAAGACGACA |
| 2237 | GGTGACCATGTGGCGTTTTAGCTT | AAGCTAAAACGCCACATGGTCACC |
| 2238 | CACGGTTGCGCACGGTACCAGAAC | GTTCTGGTACCGTGCGCAACCGTG |
| 2239 | CCTTTATTGTTTGGTCCCCTGCCC | GGGCAGGGGACCAAACAATAAAGG |
| 2240 | GTGCGCCTGCATTCTACCGTCAAT | ATTGACGGTAGAATGCAGGCGCAC |
| 2241 | GTTTACGTTGATGGCTTGCCGCCG | CGGCGGCAAGCCATCAACGTAAAC |
| 2242 | CCGTCGGTGGTAGGACGTGAATGT | ACATTCACGTCCTACCACCGACGG |
| 2243 | TGATCGCCCCAGAATCCCTGTGCT | AGCACAGGGATTCTGGGGCGATCA |
| 2244 | AAGCAGCCAAAAATCGGTTGCTTT | AAAGCAACCGATTTTTGGCTGCTT |
| 2245 | CGACGGGACTTAGTAGCAGGGCCT | AGGCCCTGCTACTAAGTCCCGTCG |
| 2246 | CCGATTCGCGAAACGACCAAGTAG | CTACTTGGTCGTTTCGCGAATCGG |
| 2247 | CCACCCCAACTCCAATCTTTCTCA | TGAGAAAGATTGGAGTTGGGGTGG |
| 2248 | GTGCAGTAGACGACTACCGGCGTC | GACGCCGGTAGTCGTCTACTGCAC |
| 2249 | TTCGCCCATCGTATCAAGCAATTC | GAATTGCTTGATACGATGGGCGAA |
| 2250 | GAATCGCGACTACCCGTCGGGTCA | TGACCCGACGGGTAGTCGCGATTC |
| 2251 | CCAGCACTCGCCATCGGTTATAAT | ATTATAACCGATGGCGAGTGCTGG |
| 2252 | CGAACCGTAGAACTCCGGTCGGTG | CACCGACCGGAGTTCTACGGTTCG |
| 2253 | GCACCATGACAGAGCCCCAGGATG | CATCCTGGGGCTCTGTCATGGTGC |
| 2254 | TGGGCTACCGCAGAATAAGGGTGA | TCACCCTTATTCTGCGGTAGCCCA |
| 2255 | TGGCCTGTCGTGTCGAAGGAAACA | TGTTTCCTTCGACACGACAGGCCA |
| 2256 | GCCTCACCGATAGCGAGCGTTTGC | GCAAACGCTCGCTATCGGTGAGGC |
| 2257 | GTGCGCGCCGGCTAAAACGAGACA | TGTCTCGTTTTAGCCGGCGCGCAC |
| 2258 | CCGCAGACGAGTTTCTTGTGACAG | CTGTCACAAGAAACTCGTCTGCGG |
| 2259 | GTTCGCAATCGCGTGCTAGGAAGC | GCTTCCTAGCACGCGATTGCGAAC |
| 2260 | TGTTGTACACATGCATCCGGTGAA | TTCACCGGATGCATGTGTACAACA |
| 2261 | CACTGAACACGATATAAGGGCGCG | CGCGCCCTTATATCGTGTTCAGTG |
| 2262 | CGCGATGGTTCTTAGCAAGACGAT | ATCGTCTTGCTAAGAACCATCGCG |
| 2263 | TACACCAAGGAAGAAATGGGGACG | CGTCCCCATTTCTTCCTTGGTGTA |
| 2264 | CGTGCCTTGCGTTTTAGGTGCAGC | GCTGCACCTAAAACGCAAGGCACG |
| 2265 | GTCGTTTGTCTGGGCATTAACGGC | GCCGTTAATGCCCAGACAAACGAC |
| 2266 | CAGGCTCTCGTTCGGTACAAACGT | ACGTTTGTACCGAACGAGAGCCTG |
| 2267 | CGGACACTGTTTCACCAGAACCCA | TGGGTTCTGGTGAAACAGTGTCCG |
| 2268 | TACCCATGATGCGGAAGAAGCGTA | TACGCTTCTTCCGCATCATGGGTA |
| 2269 | CTGTCCTTAAGCGGATGAGAACCG | CGGTTCTCATCCGCTTAAGGACAG |
| 2270 | CGGGAGATGAGAACGGTTTTGTGC | GCACAAAACCGTTCTCATCTCCCG |
| 2271 | TAGATCGCGACTGTACTCAGGCCG | CGGCCTGAGTACAGTCGCGATCTA |
| 2272 | TAAAACAGTTCGCGCGACTGTCGT | ACGACAGTCGCGCGAACTGTTTTA |
| 2273 | CGAGGAGCTCCACATAAGCCCAAT | ATTGGGCTTATGTGGAGCTCCTCG |
| 2274 | TGGCTAGGGATGGGGAATCATCTT | AAGATGATTCCCCATCCCTAGCCA |
| 2275 | AGGATTGGGTGCCTGGATGCATTG | CAATGCATCCAGGCACCCAATCCT |
| 2276 | TGTATCTACCGGCCTGAAGCAGGT | ACCTGCTTCAGGCCGGTAGATACA |
| 2277 | TCCCTACGCGCATGACTCGCTTAC | GTAAGCGAGTCATGCGCGTAGGGA |
| 2278 | TGGTCGATCACCTGTGACAGACGC | GCGTCTGTCACAGGTGATCGACCA |
| 2279 | TGGGGGTAGTCCATGCATCAATTG | CAATTGATGCATGGACTACCCCCA |
| 2280 | CCCTGCCAGGATTACTATTCCGGA | TCCGGAATAGTAATCCTGGCAGGG |
| 2281 | TCCCGCACGGGGAATTTAAGTAGA | TCTACTTAAATTCCCCGTGCGGGA |
| 2282 | GTGATGTGCAGGAACTTCTGTCGC | GCGACAGAAGTTCCTGCACATCAC |
| 2283 | ATTTAGGCATGCATGCGCTTCTCA | TGAGAAGCGCATGCATGCCTAAAT |
| 2284 | TTCGGCGCTAGTGGACGCCGTCAA | TTGACGGCGTCCACTAGCGCCGAA |
| 2285 | GAGCTTCATCTCATCAGTTCCGCG | CGCGGAACTGATGAGATGAAGCTC |
| 2286 | GACAACTCCACTGCTCCAATCGCA | TGCGATTGGAGCAGTGGAGTTGTC |
| 2287 | GGCCAAGGATGGACCTTACGATGG | CCATCGTAAGGTCCATCCTTGGCC |
| 2288 | GGTTCCGGAATTTGTCACCGCTTC | GAAGCGGTGACAAATTCCGGAACC |
| 2289 | GCGCTGGATAGTCTGCGAGAAGCC | GGCTTCTCGCAGACTATCCAGCGC |
| 2290 | TGAGTCCAGTGCTGCCACCATGAA | TTCATGGTGGCAGCACTGGACTCA |
| 2291 | TTGAATTGGGTGTCGGAGCGTTCT | AGAACGCTCCGACACCCAATTCAA |
| 2292 | CGGCGGGCAGACAATGCTTTGAAC | GTTCAAAGCATTGTCTGCCCGCCG |
| 2293 | GGGTCTGTCAAAGAGGGTGTCTGG | CCAGACACCCTCTTTGACAGACCC |
| 2294 | CTTTGTGCAAGACGAAGCACCCTT | AAGGGTGCTTCGTCTTGCACAAAG |
| 2295 | ATCGAATTCCGAGGAGGTCTCCAT | ATGGAGACCTCCTCGGAATTCGAT |
| 2296 | TCCGACCCTCAGAGTCGACTCATT | AATGAGTCGACTCTGAGGGTCGGA |
| 2297 | ATCAACGGCCACCTCCTCGCCGAG | CTCGGCGAGGAGGTGGCCGTTGAT |
| 2298 | AGCCACGGAATAATTCCGTCCACC | GGTGGACGGAATTATTCCGTGGCT |
| 2299 | GATCGCTTGCGTATCGCAAAGACT | AGTCTTTGCGATACGCAAGCGATC |
| 2300 | TCCACGCCTTACCATCAACTGCAA | TTGCAGTTGATGGTAAGGCGTGGA |
| 2301 | GCCAAGCGATAGGCCAGAACTCAG | CTGAGTTCTGGCCTATCGCTTGGC |
| 2302 | AGCGTGTGGGTCATTTTAGCACGA | TCGTGCTAAAATGACCCACACGCT |
| 2303 | GTTATGCGCGGCTTACGAGTTCGA | TCGAACTCGTAAGCCGCGCATAAC |
| 2304 | TCTGTCCACGTAACTTGCCTGCAG | CTGCAGGCAAGTTACGTGGACAGA |
| 2305 | TCGGCAGCCAATGATCATACCTCT | AGAGGTATGATCATTGGCTGCCGA |
| 2306 | TAAGCCCGATCCGGTCCTGTGTTT | AAACACAGGACCGGATCGGGCTTA |
| 2307 | ACATGGCAGACTAACAGGCCTCGC | GCGAGGCCTGTTAGTCTGCCATGT |
| 2308 | CATGGCTGCACTCTAAGTCGAACG | CGTTCGACTTAGAGTGCAGCCATG |
| 2309 | TCTTCAACCCACGCGGAACGATTG | CAATCGTTCCGCGTGGGTTGAAGA |
| 2310 | CTCGTGTCTCCAGAGGATTGTCCC | GGGACAATCCTCTGGAGACACGAG |
| 2311 | TGAAGGCATCAACCCAGAGGATTT | AAATCCTCTGGGTTGATGCCTTCA |
| 2312 | ACAGCTCGAAGGCAGCCACATTGG | CCAATGTGGCTGCCTTCGAGCTGT |
| 2313 | ACAACGAGTACCGCGACAGAAGGG | CCCTTCTGTCGCGGTACTCGTTGT |
| 2314 | ATAACCGAAAAACCAGCCTGCGAT | ATCGCAGGCTGGTTTTTCGGTTAT |
| 2315 | ACAACTCAGCACTTTCGACGTCCA | TGGACGTCGAAAGTGCTGAGTTGT |
| 2316 | CGGGTTACTGGGTATCACCAATGC | GCATTGGTGATACCCAGTAACCCG |
| 2317 | CATCGGTTATCGCTGCACGCGCGT | ACGCGCGTGCAGCGATAACCGATG |
| 2318 | GAAGGAATCCCGGATAGTCCGTGG | CCACGGACTATCCGGGATTCCTTC |
| 2319 | GCATGGTCTCAGCCAAAGAACCTG | CAGGTTCTTTGGCTGAGACCATGC |
| 2320 | AGCCTGCGACGTTTCCCGACAGAC | GTCTGTCGGGAAACGTCGCAGGCT |
| 2321 | AAGAAAGGCGCACGGGATCGATAT | ATATCGATCCCGTGCGCCTTTCTT |
| 2322 | TGTCGCGAAGCCAACTTTCAGTAA | TTACTGAAAGTTGGCTTCGCGACA |
| 2323 | GCGGCATGCAAGGTAGGTCTGGAT | ATCCAGACCTACCTTGCATGCCGC |
| 2324 | GGTGGCCATCTCCTCGAATTGCAT | ATGCAATTCGAGGAGATGGCCACC |
| 2325 | GCGTGCATAAGTTGCACATTGTGC | GCACAATGTGCAACTTATGCACGC |
| 2326 | TTGAGGTAGCGTTTTCGCGCATAT | ATATGCGCGAAAACGCTACCTCAA |
| 2327 | ATCCCACTTGTGAGAGGGCGCATT | AATGCGCCCTCTCACAAGTGGGAT |
| 2328 | CGGTCAGCGAGCAGACATCAACCT | AGGTTGATGTCTGCTCGCTGACCG |
| 2329 | GCGTATCTTCGGGTCGAACACTTG | CAAGTGTTCGACCCGAAGATACGC |
| 2330 | ATGCCATTGAACTCGCACTTTGCG | CGCAAAGTGCGAGTTCAATGGCAT |
| 2331 | CGATTCCCATCATAATGTGGGTCC | GGACCCACATTATGATGGGAATCG |
| 2332 | CAATTTGGATAATCCAGCCACGCC | GGCGTGGCTGGATTATCCAAATTG |
| 2333 | CGGCTTACCCTATGATTGCGTGCA | TGCACGGAATCATAGGGTAAGCCG |
| 2334 | GGTGGACCATGCGCTGTGGTATGA | TCATACCACAGCGCATGGTCCACC |
| 2335 | TATTTGTCGAAGATCGCAAGCGCC | GGCGCTTGCGATCTTCGACAAATA |
| 2336 | GTCAGTGGGTTTTGAGAGCCCGCA | TGCGGGCTCTCAAAACCCACTGAC |
| 2337 | AGGGGGTCGGGAAATCTGACAAAA | TTTTGTCAGATTTCCCGACCCCCT |
| 2338 | TGCTTGCTATCCGAAAAAAGCAGG | CCTGCTTTTTTCGGATAGCAAGCA |
| 2339 | TTATCGGATCAAATTCGGCTTCGG | CCGAAGCCGAATTTGATCCGATAA |
| 2340 | TGCAGCAACGAGTTACCCGGACTT | AAGTCCGGGTAACTCGTTGCTGCA |
| 2341 | TATACATGTCCGGAGGGGCACCCA | TGGGTGCCCCTCCGGACATGTATA |
| 2342 | TGCAAAACCGGAGGATGAACCCTT | AAGGGTTCATCCTCCGGTTTTGCA |
| 2343 | TCGGTCTAATGTCCACGCAGACAC | GTGTCTGCGTGGACATTAGACCGA |
| 2344 | ATGTGTTTGCCACGCGCTCCTATT | AATAGGAGCGCGTGGCAAACACAT |
| 2345 | TGGCGAGGCACGGCTCTAATTCGG | CCGAATTAGAGCCGTGCCTCGCCA |
| 2346 | GCGACGACCCGAGCGACTTTTACA | TGTAAAAGTCGCTCGGGTCGTCGC |
| 2347 | CTCAGAGAGTCTATCCGGCGCCCT | AGGGCGCCGGATAGACTCTCTGAG |
| 2348 | GGAACATCTCCTGGGTCCCTCAGA | TCTGAGGGACCCAGGAGATGTTCC |
| 2349 | GCAACGCAGGGAAGTACTTAGCGA | TCGCTAAGTACTTCCCTGCGTTGC |
| 2350 | TGACTTGGGCGGACAAAGAAACGC | GCGTTTCTTTGTCCGCCCAAGTCA |
| 2351 | AGATCATCGGGACGCTTCATGCTA | TAGCATGAAGCGTCCCGATGATCT |
| 2352 | CCCTTCTGACCGCTAAGGCCATAA | TTATGGCCTTAGCGGTCAGAAGGG |
| 2353 | CGTGAGCCGTGGGGTGTCTCTGTA | TACAGAGACACCCCACGGCTCACG |
| 2354 | TACCTTGGTCGTCTCCGCTTTTGT | ACAAAAGCGGAGACGACCAAGGTA |
| 2355 | TCGCCGCAAAATGCTACGTGAAAA | TTTTCACGTAGCATTTTGCGGCGA |
| 2356 | GAGTGACCTAATGGCTGCCCGACT | AGTCGGGCAGCCATTAGGTCACTC |
| 2357 | AAAGGAACTTGGCCAACCCTATGG | CCATAGGGTTGGCCAAGTTCCTTT |
| 2358 | TGTTTTCGCACTCCACCTAATCGC | GCGATTAGGTGGAGTGCGAAAACA |
| 2359 | CAATGGGTTTCATAAGGGCAGGCA | TGCCTGCCCTTATGAAACCCATTG |
| 2360 | GCCTAACACACAAGGGTCCCTCTG | CAGAGGGACCCTTGTGTGTTAGGC |
| 2361 | CGTCATGCGGTCCGAGGATCGATC | GATCGATCCTCGGACCGCATGACG |
| 2362 | CCACACGGGCACGGAGTAATATCT | AGATATTACTCCGTGCCCGTGTGG |
| 2363 | CATCAGACATAGGTCGCGTGCCGA | TCGGCACGCGACCTATGTCTGATG |
| 2364 | AGATGAAACCAAGGGAGGACGCAG | CTGCGTCCTCCCTTGGTTTCATCT |
| 2365 | GGCTACCCATAGGCTCAGCAGCAC | GTGCTGCTGAGCCTATGGGTAGCC |
| 2366 | GGCTTGTGAGGGTGTGTTCTCGAC | GTCGAGAACACACCCTCACAAGCC |
| 2367 | TGTGTTACGGCGAATGCAACAGTC | GACTGTTGCATTCGCCGTAACACA |
| 2368 | CGATAACAGGTCGCGCCGTTACTA | TAGTAACGGCGCGACCTGTTATCG |
| 2369 | TGATAAAGTGAGGCTCCAGCGCGA | TCGCGCTGGAGCCTCACTTTATCA |
| 2370 | AATTGTGCACGGATCTGCACGGCG | CGCCGTGCAGATCCGTGCACAATT |
| 2371 | GCAATGTACTGTCACCAGTGGCGA | TCGCCACTGGTGACAGTACATTGC |
| 2372 | GGCATATCGGTAACACTTGGTCGG | CCGACCAAGTGTTACCGATATGCC |
| 2373 | GGGTCTCAAACCAGCGTGGCCGCT | AGCGGCCACGCTGGTTTGAGACCC |
| 2374 | GTCTCCGGGACCATTGAGCTGGAG | CTCCAGCTCAATGGTCCCGGAGAC |
| 2375 | GGCCTTCGGCATTCAGACGGGTTG | CAACCCGTCTGAATGCCGAAGGCC |
| 2376 | CGTGATAGGCCACAGCGCTCAATT | AATTGAGCGCTGTGGCCTATCACG |
| 2377 | GGCAGGCCCGCGAGGATGATTAAC | GTTAATCATCCTCGCGGGCCTGCC |
| 2378 | CGGGTATGGTTGATAACAGCGTGG | CCACGCTGTTATCAACCATACCCG |
| 2379 | ACGACGTCCTTGGGACCGTATTGT | ACAATACGGTCCCAAGGACGTCGT |
| 2380 | CTGATATCGAGCCTGAGCCTTTCG | CGAAAGGCTCAGGCTCGATATCAG |
| 2381 | TCCCATTGGCCTGTATGCTGGCCT | AGGCCAGCATACAGGCCAATGGGA |
| 2382 | GTGTCGTCGATTGTTTCATCGACG | CGTCGATGAAACAATCGACGACAC |
| 2383 | CGAAAGCCAGTAGCCGATTGCGTG | CACGCAATCGGCTACTGGCTTTCG |
| 2384 | GGTTCGGCTTATTCCACTGCGACA | TGTCGCAGTGGAATAAGCCGAACC |
| 2385 | AGCGAGGGCTAACTTTTTAACGCG | CGCGTTAAAAAGTTAGCCCTCGCT |
| 2386 | CGGCGCTGATGACGGGACTCGATT | AATCGAGTCCCGTCATCAGCGCCG |
| 2387 | TCACAGTGCTCGGCGTAAGGACTA | TAGTCCTTACGCCGAGCACTGTGA |
| 2388 | CCCATTACGAGCACACACCATGGC | GCCATGGTGTGTGCTCGTAATGGG |
| 2389 | GGCCGCTAATCTTTACGCATCACG | CGTGATGCGTAAAGATTAGCGGCC |
| 2390 | ACGGCTTCCTAGTGTCCAGCCCTT | AAGGGCTGGACACTAGGAAGCCGT |
| 2391 | CTGTCAGGTCCTACCCAATGGCTC | GAGCCATTGGGTAGGACCTGACAG |
| 2392 | CACAGCCCATCCCACTGAACTGCT | AGCAGTTCAGTGGGATGGGCTGTG |
| 2393 | ACAAACGATACACGCAACGCTGTG | CACAGCGTTGCGTGTATCGTTTGT |
| 2394 | TGGCGGCCAGCTAGCAGGCGAAGT | ACTTCGCCTGCTAGCTGGCCGCCA |
| 2395 | ATCTCGAAACGATGCGTGCCTAAA | TTTAGGCACGCATCGTTTCGAGAT |
| 2396 | ATCTCGAGAACAGCGTGCGTGCGG | CCGCACGCACGCTGTTCTCGAGAT |
| 2397 | GAAGAAATCCGCCGACATCTACGG | CCGTAGATGTCGGCGGATTTCTTC |
| 2398 | GCGGAGCAACCTTGGCTGTTTCTA | TAGAAACAGCCAAGGTTGCTCCGC |
| 2399 | CGCGTTCCGAAGACTTGTTGTTTG | CAAACAACAAGTCTTCGGAACGCG |
| 2400 | TGACCTGAAGCCCATCCATAAGCA | TGCTTATGGATGGGCTTCAGGTCA |
| 2401 | TGGTATTCATTCCGGATAAGCGGG | CCCGCTTATCCGGAATGAATACCA |
| 2402 | GCGTTGCGGGTCATTGATGCAAAC | GTTTGCATCAATGACCCGCAACGC |
| 2403 | ACCGCTTTCTGTGTAGAGCCCTGA | TCAGGGCTCTACACAGAATGCGGT |
| 2404 | CAAATAGACAATCGCAGCTTCGGG | CCCGAAGCTGCGATTGTCTATTTG |
| 2405 | TGTCCTGACAAATCAAGGTGCAGG | CCTGCACCTTGATTTGTCAGGACA |
| 2406 | AAATTGCACTCGCGGAGATTTCCT | AGGAAATCTCCGCGAGTGCAATTT |
| 2407 | TGACGCCCATTTCTATATGGTGCA | TGCACCATATAGAAATGGGCGTCA |
| 2408 | TGTTCCGACAGGGCACTGCTAGAC | GTCTAGCAGTGCCCTGTCGGAACA |
| 2409 | TCGCTGGCTTGGGAAGGCCTTCGT | ACGAAGGCCTTCCCAAGCCAGCGA |
| 2410 | GTGCACCTCCGTTGGCGTAGAATG | CATTCTACGCCAACGGAGGTGCAC |
| 2411 | CTCATTTGGGACCGATCGGGTTGC | GCAACCCGATCGGTCCCAAATGAG |
| 2412 | GCCAGTGTCTGTCAATGGATGGGA | TCCCATCCATTGACAGACACTGGC |
| 2413 | TTGCCCGGCAGGTTCTGTGTAATG | CATTACACAGAACCTGCCGGGCAA |
| 2414 | ACCCGCGAACCGAGACGCACTTCT | AGAAGTGCGTCTCGGTTCGCGGGT |
| 2415 | TCCGTGCGATTGGTCAAGGTTGAT | ATCAACCTTGACCAATCGCACGGA |
| 2416 | AGGGCGTCTCGGTTGAACCTCGGT | ACCGAGGTTCAACCGAGACGCCCT |
| 2417 | TGACCGTTCAAAGAGCAAGCCAAC | GTTGGCTTGCTCTTTGAACGGTCA |
| 2418 | ACACTCACCTGCTGTCCCTGCTGA | TCAGCAGGGACAGCAGGTGAGTGT |
| 2419 | GCGTTTAACTCCTTGGGTGGTGGT | ACCACCACCCAAGGAGTTAAACGC |
| 2420 | CGCCTGCGCAGGTAACTCTCCGCA | TGCGGAGAGTTACCTGCGCAGGCG |
| 2421 | AATCGAATTTCCCAGCGGCTGTTT | AAACAGCCGCTGGGAAATTCGATT |
| 2422 | AAGCAGGTGGGATCCTGGGGATCA | TGATCCCCAGGATCCCACCTGCTT |
| 2423 | AATCCCAGACTCGCTCTTCGTGCT | AGCACGAAGAGCGAGTCTGGGATT |
| 2424 | ACGGTTATAAGGGCCGGCTGCGAC | GTCGCAGCCGGCCCTTATAACCGT |
| 2425 | TACGAGAGCGGGCTTAGACGTCGC | GCGACGTCTAAGCCCGCTCTCGTA |
| 2426 | GCGATTTTGACCCACGGTTATCGA | TCGATAACCGTGGGTCAAAATCGC |
| 2427 | AGCTGTATAATTTGGATGGCGCGA | TCGCGCCATCCAAATTATACAGCT |
| 2428 | TCCGCGAGTCTTAGCCGATTGAAC | GTTCAATCGGCTAAGACTCGCGGA |
| 2429 | GGCATGAGCTCCGTAAGCCGATAG | CTATCGGCTTACGGAGCTGATGCC |
| 2430 | TGTTATTGGCAGTTCGAGCGACAG | CTGTCGCTCGAACTGCCAATAACA |
| 2431 | GCGAGCCTTTTTGCTTGGGAAGAG | CTCTTCCCAAGCAAAAAGGCTCGC |
| 2432 | AGAAGAAAAGGTCAGCGTCGACGA | TCGTCGACGCTGACCTTTTCTTCT |
| 2433 | CGGGTCGACCCTTGAAGCATAACC | GGTTATGCTTCAAGGGTCGACCCG |
| 2434 | CTCGGTTTTCACAAACTTACCGCG | CGCGGTAAGTTTGTGAAAACCGAG |
| 2435 | GCAGTCCTATCCGGAGCCTGACAA | TTGTCAGGCTCCGGATAGGACTGC |
| 2436 | AAGGTGCGCTATTTGTTGTCGGTC | GACCGACAACAAATAGCGCACCTT |
| 2437 | AGTGGAATCCATGCCGACACCTGA | TCAGGTGTCGGCATGGATTCCACT |
| 2438 | TACAGGCGTAATTCCTGCGAGGGA | TCCCTCGCAGGAATTACGCCTGTA |
| 2439 | CCGAAGTGCGAGAAGCACGTTGTT | AACAACGTGCTTCTCGCACTTCGG |
| 2440 | AAGGACTGGTATGGCCGGAGCTTT | AAAGCTCCGGCCATACCAGTCCTT |
| 2441 | GGACACCGCCAACCTCATAGTTGC | GCAACTATGAGGTTGGCGGTGTCC |
| 2442 | AATGGTGTTCGCCTGGACTACCAC | GTGGTAGTCCAGGCGAACACCATT |
| 2443 | TAGGAAAGCGTACACGGGAATCCG | CGGATTCCCGTGTACGCTTTCCTA |
| 2444 | TCTCACCCCAATGATGAGGACGTC | GACGTCCTCATCATTGGGGTGAGA |
| 2445 | CGTGTCCGTGTGACACTGTCCATG | CATGGACAGTGTCACACGGACACG |
| 2446 | TCCAGGCTGTTGCGGATACGGTAG | CTACCGTATCCGCAACAGCCTGGA |
| 2447 | GTAGGCAAAATGGTCGCGATCAAT | ATTGATCGCGACCATTTTGGCTAC |
| 2448 | ATCTCCGTGGACCCGATTGTGACA | TGTCACAATGGGGTCCACGGAGAT |
| 2449 | GAATATGCCGTCAACGCTATGGGC | GCCCATAGCGTTGACGGCATATTC |
| 2450 | TTCCGGAAGCGTTTGGTAACTTTG | CAAAGTTACCAAACGCTTCCGGAA |
| 2451 | TTCGATAGGAATACCAGGGCCTGG | CCAGGCCCTGGTATTCCTATCGAA |
| 2452 | GGCCATTTGAGGAGGATTATGCAA | TTGCATAATCCTCCTCAAATGGCC |
| 2453 | ACCTTCTGACCTGGACTTTTGGCG | CGCCAAAAGTCCAGGTCAGAAGGT |
| 2454 | GACCAATCCGCAGTTGAGCAACAG | CTGTTGCTCAACTGCGGATTGGTC |
| 2455 | TCGGCCACTCACCATGAGTGTAGG | CCTACACTCATGGTGAGTGGCCGA |
| 2456 | AGCGCTCACATGTTCGAAAACGGG | CCCGTTTTCGAACATGTGAGCGCT |
| 2457 | TAACGCAAAGGCGCGATCCTCGCT | AGCGAGGATCGCGCCTTTGCGTTA |
| 2458 | TGGGTGGGCCAAATATTACTGCAA | TTGCAGTAATATTTGGCCCACCCA |
| 2459 | GTCCTCGAAAGGGGCATCCAAACA | TGTTTGGATGCCCCTTTCGAGGAC |
| 2460 | CCCATCTGGTGGGAGGCGTTATCA | TGATAACGCCTCCCACCAGATGGG |
| 2461 | GTGCGCGGTCTGCAAACTCGCCAT | ATGGCGAGTTTGCAGACCGCGCAC |
| 2462 | TGTGTTGCCAACCCTAGGTCATCA | TGATGACCTAGGGTTGGCAACACA |
| 2463 | CTGATGCTGTTCTCGTCGGTTGAC | GTCAACCGACGAGAACAGCATCAG |
| 2464 | AAGCTGCAAAAGGTGAGCGTGGCA | TGCCACGCTCACCTTTTGCAGCTT |
| 2465 | TCTGACGCGTGCTTGGGAGTCTAT | ATAGACTCCCAAGCACGCGTCAGA |
| 2466 | GAATTACTTGGAGGCGCCGTGCAA | TTGCACGGCGCCTCCAAGTAATTC |
| 2467 | GATTCTTCCCGACCTAGGTTGGCC | GGCCAACCTAGGTCGGGAAGAATC |
| 2468 | CGCAGCGTATCCCATGTTGCTTGA | TCAAGCAACATGGGATACGCTGCG |
| 2469 | GAGATGGAATTGTTCGCCCAAAGA | TCTTTGGGCGAACAATTCCATCTC |
| 2470 | GATGCCTGGATCGGTCTAGCGTCA | TGACGCTAGACCGATCCAGGCATC |
| 2471 | GCAGCGACTGCTAAGCTATCTCGG | CCGAGATAGCTTAGCAGTCGCTGC |
| 2472 | AGGGCTAATTTACATCGCCTTGCC | GGCAAGGCGATGTAAATTAGCCCT |
| 2473 | AAGTGCACATCCTCACGAAGCGAT | ATCGCTTCGTGAGGATGTGCACTT |
| 2474 | TCAGGCAGCCGTAATTAAATGCGC | GCGCATTTAATTACGGCTGCCTGA |
| 2475 | CCACTGGGGAAATCGCACTGTTGG | CCAACAGTGCGATTTCCCCAGTGG |
| 2476 | TTGTCCAAAGCCACCTACGACAGA | TCTGTCGTAGGTGGCTTTGGACAA |
| 2477 | TGGGCGGAATAGATTGGGTGTCTT | AAGACACCCAATCTATTCCGCCCA |
| 2478 | TAGAATTCGCCTCTTCTAGCCGCC | GGCGGCTAGAAGAGGCGAATTCTA |
| 2479 | CATTACTTCCTGCAGATGCGATGC | GCATCGCATCTGCAGGAAGTAATG |
| 2480 | GGAAATGCTAGCTGGGGTAATCGC | GCGATTACCCCAGCTAGCATTTCC |
| 2481 | GCCGCCACTTGCGAATCTACATCT | AGATGTAGATTCGCAAGTGGCGGC |
| 2482 | ACAATAGCGGACAGCTCGCCAGAT | ATCTGGCGAGCTGTCCGCTATTGT |
| 2483 | AGTTAGGCTCTCGGTGCGGTCCAT | ATGGACCGCACCGAGAGCCTAACT |
| 2484 | TGGGCCTGAGAAGCGGTTAATAGG | CCTATTAACCGCTTCTCAGGCCCA |
| 2485 | ACGCTCTGAGCGACGCCTATCGTA | TACGATAGGCGTCGCTCAGAGCGT |
| 2486 | CCTGGTGATCGTGTCCCAGACTCA | TGAGTCTGGGACACGATCACCAGG |
| 2487 | GCGTGTCCATTCGCTTGAGGTTTC | GAAACCTCAAGCGAATGGACACGC |
| 2488 | ATCCTGAACGGCGATGACCACCAC | GTGGTGGTCATCGCCGTTCAGGAT |
| 2489 | TTACGTTTCTCACCGATCAACGCC | GGCGTTGATCGGTGAGAAACGTAA |
| 2490 | GCCGTCTTGAGTGGCTAAAAGGCA | TGCCTTTTAGCCACTCAAGACGGC |
| 2491 | ATCTACGATGCGGCTCGAAGTGTT | AACACTTCGAGCCGCATCGTAGAT |
| 2492 | AACCAAGACTCGTCCCCAAACGTT | AACGTTTGGGGACGAGTCTTGGTT |
| 2493 | AACTGCGGTGGTGGAGGCAGGTGC | GCACCTGCCTCCACCACCGCAGTT |
| 2494 | TGCGATCTTCTCCACCTACAGCGC | GCGCTGTAGGTGGAGAAGATCGCA |
| 2495 | AGGCGCTTAGAACCGTGAAGGCAG | CTGCCTTCACGGTTCTAAGCGCCT |
| 2496 | TGGAAAATTTTGGGAAACGCTGGA | TCCAGCGTTTCCCAAAATTTTCCA |
| 2497 | CCAGCGCCGCACCTTCTCCAATAG | CTATTGGAGAAGGTGCGGCGCTGG |
| 2498 | TAGACGGCTGGCGAATCTTACGGT | ACCGTAAGATTCGCCAGCCGTCTA |
| 2499 | TACCATACAAGAGAACGAGCCGCA | TGCGGCTCGTTCTCTTGTATGGTA |
| 2500 | GTAGCCGAGAGCAATTTTCACCGC | GCGGTGAAAATTGCTCTCGGCTAC |
| 2501 | GCAAACTCCCCTGCCCTTTAGCCT | AGGCTAAAGGGCAGGGGAGTTTGC |
| 2502 | ATCCCGCTGATAACCGCCAGGATA | TATCCTGGCGGTTATCAGCGGGAT |
| 2503 | AGTCTCAGTTCGGCGCAACGGTAG | CTACCGTTGCGCCGAACTGAGACT |
| 2504 | AACCTACAGTCGCCGCAATGCATT | AATGCATTGCGGCGACTGTAGGTT |
| 2505 | ATACACGTTTCAGCCGGCAACAAT | ATTGTTGCCGGCTGAAACGTGTAT |
| 2506 | ACGACGGGACGTGCCCTCGTTGAT | ATCAACGAGGGCACGTCCCGTCGT |
| 2507 | AAGTCCAAACTCGAATGGGGCAGT | ACTGCCCCATTCGAGTTTGGACTT |
| 2508 | GATTTATTGGCGCGGTAACGACCT | AGGTCGTTACCGCGCCAATAAATC |
| 2509 | TGTTTTCAGAGGCTACCCTGCCAT | ATGGCAGGGTAGCCTCTGAAAACA |
| 2510 | ACGGTCTCAGGGAAATGCGATCTC | GAGATCGCATTTCCCTGAGACCGT |
| 2511 | GACTTGAAACCGCCTATGCCCACA | TGTGGGCATAGGCGGTTTCAAGTC |
| 2512 | CGATCGGTTGTGTGCTGTCTTACC | GGTAAGACAGCACACAACCGATCG |
| 2513 | AGTAGCACAATGCCTCATTTCCGC | GCGGAAATGAGGCATTGTGCTACT |
| 2514 | CTCGCTATCTACGCGTCTCCGAAA | TTTCGGAGACGCGTAGATAGCGAG |
| 2515 | AGCCCGTTACGGCATCTAGGATTC | GAATCCTAGATGCCGTAACGGGCT |
| 2516 | TCGCGATGGCGAGAGTTCAGAATA | TATTCTGAACTCTCGCCATCGCGA |
| 2517 | TTACAGGATTCCAAAACCCGCAAA | TTTGCGGGTTTTGGAATCCTGTAA |
| 2518 | CGGTACCAACGCGCGGGCATATGA | TCATATGCCCGCGCGTTGGTACCG |
| 2519 | TGCCAGTATTATCCGTGCCAGCCG | CGGCTGGCACGGATAATACTGGCA |
| 2520 | ATTTCAGACCTCGGGACAACCTGG | CCAGGTTGTCCCGAGGTCTGAAAT |
| 2521 | GAAGTGCGCGTAACTTAGGGAGCC | GGCTCCCTAAGTTACGCGCACTTC |
| 2522 | TTGGCCAGGTCATCACTCTGCCAT | ATGGCAGAGTGATGACCTGGCCAA |
| 2523 | ATCGGCCGGTATTAGCTGCCCTCC | GGAGGGCAGCTAATACCGGCCGAT |
| 2524 | CGCAGGTAAGGCCGAGCAATGTTT | AAACATTGCTCGGCCTTACCTGCG |
| 2525 | TTGGGAACGTGCTAGGCGGCCCTC | GAGGGCCGCCTAGCACGTTCCCAA |
| 2526 | CATCTCGGCACACTGGTGCTGTAT | ATACAGCACCAGTGTGCCGAGATG |
| 2527 | ACGCGTAAATCAACGACGTGGTCG | CGACCACGTCGTTGATTTACGCGT |
| 2528 | CGTAGGTGGTAAATGTTGGCCCAG | CTGGGCCAACATTTACCACCTACG |
| 2529 | TTCGAGCCAGAATAAAACGGTTGG | CCAACCGTTTTATTCTGGCTCGAA |
| 2530 | AGAGATATTCGGCCTCGGTCGAGA | TCTCGACCGAGGCCGAATATCTCT |
| 2531 | CGACAAAGTTTCTCGCGAGCAACT | AGTTGCTCGCGAGAAACTTTGTCG |
| 2532 | ATTGCCGCGTCTCGTATCAAAAGA | TCTTTTGATACGAGACGCGGCAAT |
| 2533 | CGGAGAATGGATGCAGGTTCTTCG | CGAAGAACCTGCATCCATTCTCCG |
| 2534 | TATAATCATTTGCGACTCGCCCCA | TGGGGCGAGTCGCAAATGATTATA |
| 2535 | AATTTTCCCCGATTTGAAGAAGCG | CGCTTCTTCAAATCGGGGAAAATT |
| 2536 | TCGCATACTTCGTCGGCGAGTATT | AATACTCGCCGACGAAGTATGCGA |
| 2537 | CGTGAGCCGTTCTCATCCAAGCGG | CCGCTTGGATGAGAACGGCTCACG |
| 2538 | GCAGAATCGAATTGGGGTGGGTTT | AAACCCACCCCAATTCGATTCTGC |
| 2539 | CTCTCGGTTTCTCAACCGAGCTCG | CGAGCTCGGTTGAGAAACCGAGAG |
| 2540 | GACCAGTTAGTGCAATGGTTGGCG | CGCCAACCATTGCACTAACTGGTC |
| 2541 | TTCTCGCACAGCTAGTCAGCCGAT | ATCGGCTGACTAGCTGTGCGAGAA |
| 2542 | CCAAGTCTTGCGTGAGCGATCCTG | CAGGATCGCTCACGCAAGACTTGG |
| 2543 | GCGAAAGTGGCTCGTATTTCTCCA | TGGAGAAATACGAGCCACTTTCGC |
| 2544 | CCTCGGGACTGTCCGACTGAAAAA | TTTTTCAGTCGGACAGTCCCGAGG |
| 2545 | AGGCGAGTGTACGGCTCATCCATG | CATGGATGAGCCGTACACTCGCCT |
| 2546 | GCGGCTCTGCCTACGATATTCACA | TGTGAATATCGTAGGCAGAGCCGC |
| 2547 | TGCACCTGTCTGTAGATTTGCGGT | ACCGCAAATCTACAGACAGGTGCA |
| 2548 | CATAAAGCACGGACGCGACTTGAT | ATCAAGTCGCGTCCGTGCTTTATG |
| 2549 | CCCTCAACGTAGGGCGTGACTTTC | GAAAGTCACGCCCTACGTTGAGGG |
| 2550 | GGGTCATCGTGCAGTTATGCCGTA | TACGGCATAACTGCACGATGACCC |
| 2551 | CCCGGATAATCCTTTGTCCAGCCG | CGGCTGGACAAAGGATTATCCGGG |
| 2552 | TCCGATAAGCGAACTCACATGGGT | ACCCATGTGAGTTCGCTTATCGGA |
| 2553 | CCTGCTGGTTCGGTCGTAAGCGAA | TTCGCTTACGACCGAACCAGCAGG |
| 2554 | GAGGCACCAATCGGTCTGAAAATG | CATTTTCAGACCGATTGGTGCCTC |
| 2555 | TACGAAAATGGTTGCGCCGGGTCT | AGACCCGGCGCAACCATTTTCGTA |
| 2556 | AATTGCCGGAAGCAGTCAGAATCG | CGATTCTGACTGCTTCCGGCAATT |
| 2557 | CCGAATCAGCCGTATTTGCTGGAA | TTCCAGCAAATACGGCTGATTCGG |
| 2558 | CCCGCTTATCTGTACTCGATCGCA | TGCGATCGAGTACAGATAAGCGGG |
| 2559 | TTTTGGGGATCCCTATTAGGCGCA | TGCGCCTAATAGGGATCCCCAAAA |
| 2560 | AGTGACAGCGCTCACCACGGTCCC | GGGACCGTGGTGAGCGCTGTCACT |
| 2561 | CCATGAGTGTTTCGGGACATCGTA | TACGATGTCCCGAAACACTCATGG |
| 2562 | GCCACATTCTGCTACCTCCGTGTT | AACACGGAGGTAGCAGAATGTGGC |
| 2563 | TCCTGTGCTTTGTGACGTGCTAGG | CCTAGCACGTCACAAAGCACAGGA |
| 2564 | GACCGCATATACACCTGATGGGCC | GGCCCATCAGGTGTATATGCGGTC |
| 2565 | GTAGGCCCGTCGTTAACCATCTCA | TGAGATGGTTAACGACGGGCCTAC |
| 2566 | CGGCTCGCGAAATGGAGTTTAGCG | CGCTAAACTCCATTTCGCGAGCCG |
| 2567 | GCTGATCGGCTTTTCACCGCTATA | TATAGCGGTGAAAAGCCGATCAGC |
| 2568 | TATCAAATCGTTGGCACGCGACTA | TAGTCGCGTGCCAACGATTTGATA |
| 2569 | TTGGCGAGGATCCCTAGGCGTACT | AGTACGCCTAGGGATCCTCGCCAA |
| 2570 | AAGTCCTGAGGCCGTTCGGTTTCT | AGAAACCGAACGGCCTCAGGACTT |
| 2571 | ACTCCGGACATCTCGGCCAGAGAT | ATCTCTGGCCGAGATGTCCGGAGT |
| 2572 | CCAAGGGGAACACAGGATCGTAGA | TCTACGATCCTGTGTTCCCCTTGG |
| 2573 | GTGGCCTAAATCCGCCTTCTCAAC | GTTGAGAAGGCGGATTTAGGCCAC |
| 2574 | CACTCCGTCTCGTCCATTAATGCG | CGCATTAATGGACGAGACGGAGTG |
| 2575 | TCAAGAACCCAGTGCCGGTCAGCA | TGCTGACCGGCACTGGGTTCTTGA |
| 2576 | GAATCAATTTTCCAGGGACGGGAC | GTCCCGTCCCTGGAAAATTGATTC |
| 2577 | ATCGGTGTGCTGGAGCGCCAGAGT | ACTCTGGCGCTCCAGCACACCGAT |
| 2578 | GCCTCTCCTATGACGATGACCCAC | GTGGGTCATCGTCATAGGAGAGGC |
| 2579 | TGGGCGCGCTTTTAAGACTACATC | GATGTAGTCTTAAAAGCGCGCCCA |
| 2580 | CGTTGGGTACCGTTCTATCAACCG | CGGTTGATAGAACGGTACCCAACG |
| 2581 | GCAGTGAGCTGGGTTCAATGCTTC | GAAGCATTGAACCCAGCTCACTGC |
| 2582 | CATCATCCACACAGGCAGGTGTGT | ACACACCTGCCTGTGTGGATGATG |
| 2583 | AGACAAAGGTCCCCATTGCGAAAT | ATTTCGCAATGGGGACCTTTGTCT |
| 2584 | ATACTCGTCGACGAGAAGCGGAAA | TTTCCGCTTCTCGTCGACGAGTAT |
| 2585 | GCAGAATGTGTTGTCTTCGCAGCC | GGCTGCGAAGACAACACATTCTGC |
| 2586 | CACCATGCCTTCATCTTGGCCTAG | CTAGGCCAAGATGAAGGCATGGTG |
| 2587 | ACTCTTCAACGCCAGGTTAAGCCA | TGGCTTAACCTGGCGTTGAAGAGT |
| 2588 | GCGACCTGCGGCGTGTGTATTCTC | GAGAATACACACGCCGCAGGTCGC |
| 2589 | TCGGTGTATGCACCCTTTCTCCAT | ATGGAGAAAGGGTGCATACACCGA |
| 2590 | ACCGTCGAATCTTGCGGCCAATGT | ACATTGGCCGCAAGATTCGACGGT |
| 2591 | TAATGCATGCTCCCGGCTCACGTT | AACGTGAGCCGGGAGCATGCATTA |
| 2592 | TCTGTACACACCACGTCGTGCACA | TGTGCACGACGTGGTGTGTACAGA |
| 2593 | TATGGGGTTGTCAGACGACACCTA | TAGGTGTCGTCTGACAACCCCATG |
| 2594 | AATCTGATGCTCGCTGTAGGACGG | CCGTCCTACAGCGAGCATCAGATT |
| 2595 | TCGAAACCGCGGGAAAGGGTAAAA | TTTTACCCTTTCCCGCGGTTTCGA |
| 2596 | TGGGGGACGGGCGTCTAATCCTCC | GGAGGATTAGACGCCCGTCCCCCA |
| 2597 | AGGCATGCACCCATGCTGCCAGAG | CTCTGGCAGCATGGGTGCATGCCT |
| 2598 | TCCCAATGGCCTGTCAAGCATAAA | TTTATGCTTGACAGGCCATTGGGA |
| 2599 | GAACCTGAGCCTTTGCTAGCACGA | TCGTGCTAGCAAAGGCTCAGGTTC |
| 2600 | CGAATTGATAGCGTTACGGGCGAA | TTCGCCCGTAACGCTATCAATTCG |
| 2601 | TTGCACGCGCGCGAACGACTATTC | GAATAGTCGTTCGCGCGCGTGCAA |
| 2602 | TGCGGTGAAGCAGTCCAAGGTCAG | CTGACCTTGGACTGCTTCACCGCA |
| 2603 | TGAGGACCATCCAATGGATCGGTT | AACCGATCCATTGGATGGTCCTCA |
| 2604 | TCGGTGATTGGTAATTTGGATCCG | CGGATCCAAATTACCAATCACCGA |
| 2605 | GCGGGCAGGTAGTTTGACTGGATG | CATCCAGTCAAACTACCTGCCCGC |
| 2606 | CAAGCACAAGCCCATGAAATTTCA | TGAAATTTCATGGGCTTGTGCTTG |
| 2607 | CGGTACAGCGGATAGCCAAGGATA | TATCCTTGGCTATCCGCTGTACCG |
| 2608 | CCATGCTCTTCGCTGCAGCATACT | AGTATGCTGCAGCGAAGAGCATGG |
| 2609 | CGCGGCAAAGATTAATTCCCGGCG | CGCCGGGAATTAATCTTTGCCGCG |
| 2610 | GAAGACCCGTCCGGGTTTCCATAC | GTATGGAAACCCGGACGGGTCTTC |
| 2611 | CTGGCAAGGAGGATGTGGCTCGTG | CACGAGCCACATCCTCCTTGCCAG |
| 2612 | CTGTGCAGGGGGTGGCTCTGTTGA | TCAACAGAGCCACCCCCTGCACAG |
| 2613 | TTCAATAATGATCACGAGGCCCCA | TGGGGCCTCGTGATCATTATTGAA |
| 2614 | TGGTGATGCGAAGCCTTACCTTTG | CAAAGGTAAGGCTTCGCATCACCA |
| 2615 | CTGCCACCATCTACGGCGCAGTCT | AGACTGCGCCGTAGATGGTGGCAG |
| 2616 | TTTGCCCAGCTCTCGCAGAAGTTA | TAACTTCTGCGAGAGCTGGGCAAA |
| 2617 | AATTCAGACGCCACATCGACGGTC | GACCGTCGATGTGGCGTCTGAATT |
| 2618 | CCGTGGTCTGCCTCGATTACCTAC | GTAGGTAATCGAGGCAGACCACGG |
| 2619 | GGCGAGGAATTTCGGAACCTTATG | CATAAGGTTCCGAAATTCCTCGCC |
| 2620 | ATCCGATGATCAGATACCGGCTGG | CCAGCCGGTATCTGATCATCGGAT |
| 2621 | CCATAGACTAGCGCCAGAGTGCCC | GGGCACTCTGGCGCTAGTGTATGG |
| 2622 | TGTGGACCTAGAAAATTGCCAGCC | GGCTGGCAATTTTCTAGGTCCACA |
| 2623 | GAATAATCATCGCGGTCCTCATGG | CCATGAGGACCGCGATGATTATTC |
| 2624 | GGGATTGGCTCTTGGTTGGAAGAA | TTCTTCCAACCAAGAGCCAATCCC |
| 2625 | ATTGTGCTTCCTCGAACTGGGAAA | TTTCCCAGTTCGAGGAAGCACAAT |
| 2626 | TGCCCCACCCCGTAAGTCAATAAT | ATTATTGACTTACGGGGTGGGGCA |
| 2627 | TCAGGACCGACGGTGCACTTAGTG | CACTAAGTGCACCGTCGGTCCTGA |
| 2628 | CCAGCCGTCACAGTGCAATTTCCG | CGGAAATTGCACTGTGACGGCTGG |
| 2629 | CTTAAAGAGGCGCGAAGCACAACA | TGTTGTGCTTCGCGCCTCTTTAAG |
| 2630 | TACCGCTCGTCGCGATCACAATGA | TCATTGTGATCGCGACGAGCGGTA |
| 2631 | CCGAGTGCGCGAAGTGTCTATGTG | CACATAGACACTTCGCGCACTCGG |
| 2632 | GCACCAGTGCCCGATCAAAACGTA | TACGTTTTGATCGGGCACTGGTGC |
| 2633 | TGCAGGCTTCTCAACGGCTGGGAG | CTCCCAGCCGTTGAGAAGCCTGCA |
| 2634 | CTCCGTACGTATCCCGCGTGATAC | GTATCACGCGGGATACGTACGGAG |
| 2635 | GGAAGTGCAACTTAAAGCCCCGCC | GGCGGGGCTTTAAGTTGCACTTCC |
| 2636 | CGAACCGGCAGTCGATCGTTGCAT | ATGCAACGATCGACTGCCGGTTCG |
| 2637 | CCGTTAGTGGTCGACAGTTCGGTT | AACCGAACTGTCGACCACTAACGG |
| 2638 | TCAGGCTACGCCCTCAGCACTACA | TGTAGTGCTGAGGGCGTAGCCTGA |
| 2639 | TATACGGGCCGAGGTCCGTATTCG | CGAATACGGACCTCGGCCCGTATA |
| 2640 | CCAACGTGTGACGAAGGGCCATTG | CAATGGCCCTTCGTCACACGTTGG |
| 2641 | CTGCTCAGCGGTGCTTGAAAGACA | TGTCTTTCAAGCACCGCTGAGCAG |
| 2642 | GGAGATTGACTTCGCGTTTCACCA | TGGTGAAACGCGAAGTCAATCTCC |
| 2643 | ATGGTTCAGAAGGTTCGTCGGGTT | AACCCGACGAACCTTCTGAACCAT |
| 2644 | GAGTGGAGCATTCTCGGCCCTCAA | TTGAGGGCCGAGAATGCTCCACTC |
| 2645 | TGGATTGGAACCAATCCCGCACAA | TTGTGCGGGATTGGTTCCAATCCA |
| 2646 | TGCTCTTGTGGTCACTCGAGAGGA | TCCTCTCGAGTGACCACAAGAGCA |
| 2647 | TTGGGAGCACGGTTACCGCCTGTG | CACAGGCGGTAACCGTGCTCCCAA |
| 2648 | CAACGCGAGCTAACGGTAGTTTCG | CGAAACTACCGTTAGCTCGCGTTG |
| 2649 | AACGCTGAGCGCTCACCTTCACCT | AGGTGAAGGTGAGCGCTCAGCGTT |
| 2650 | CCGTCGTAGATCTGGAGGCTTCAA | TTGAAGCCTCCAGATCTACGACGG |
| 2651 | GGATGGCATGGGCACACTGTAACC | GGTTACAGTGTGCCCATGCCATCC |
| 2652 | TCGCTCGTAGATATCCTTCACGCC | GGCGTGAAGGATATCTACGAGCGA |
| 2653 | GGAGCAATACCGCGTCCAAAACAC | GTGTTTTGGACGCGGTATTGCTCC |
| 2654 | TTGTTCAGACTTAGGCGCTGCCCA | TGGGCAGCGCCTAAGTCTGAACAA |
| 2655 | CGGCGGTACTCTTTCCACTGTCCT | AGGACAGTGGAAAGAGTACCGCCG |
| 2656 | AAGACGATTGCCCACGTGCCAGAG | CTCTGGCACGTGGGCAATCGTCTT |
| 2657 | AGGTGAGCGCAGGCATATTGCAGT | ACTGCAATATGCCTGCGCTCACCT |
| 2658 | CTCGGGCCTGTACAGCAAAGCCGT | ACGGCTTTGCTGTACAGGCCCGAG |
| 2659 | TGCGCGCTAGTGCTGCCTATGATC | GATCATAGGCAGCACTAGCGCGCA |
| 2660 | CCATCCTTTGCCTTGAGGGTTGGC | CTTACCCTCAAGGCATTAGGATGG |
| 2661 | AACAACAGCGTAAGACGGACAGGG | CCCTGTCCGTCTTACGCTGTTGTT |
| 2662 | GAGGCGGTCGAGGCTCACAATATT | AATATTGTGAGCCTCGACCGCCTC |
| 2663 | CGAGGTTAGACGCCTATGACCCAC | GTGGGTCATAGGCGTCTAACCTCG |
| 2664 | AACTTGCTATACCGGGCGCAGCAA | TTGCTGCGCCCGGTATAGCAAGTT |
| 2665 | CGCGGTGAATCGCATACACAGCGC | GCGCTGTGTATGCGATTCACCGCG |
| 2666 | CACCGAATCAAGCCATATGGCTCT | AGAGCCATATGGCTTGATTCGGTG |
| 2667 | TTCACAGCTATCCTAGGCGCTGCC | GGCAGCGCCTAGGATAGCTGTTAA |
| 2668 | AGAAGCGCGAAGTGTACCCCGCAT | ATGCGGGGTACACTTCGCGCTTCT |
| 2669 | TGCATGGTATTTGCGTGCGATAGG | CCTATCGCACGCAAATACCATGCA |
| 2670 | GGCCGGACCTATGTGAGATGGAAA | TTTCCATCTCACATAGGTCCGGCC |
| 2671 | TCAACCTGAGTCCTGATCCCAAGC | GCTTGGGATCAGGACTCAGGTTGA |
| 2672 | TGCTTACCGTTCAGGGAGGCGTGT | ACACGCCTCCCTGAACGGTAAGCA |
| 2673 | GGAGAGTTACGCGATGAGCCACCT | AGGTGGCTCATCGCGTAACTCTCC |
| 2674 | CGGTATGCGGTGTACAGCTTTCGT | ACGAAAGCTGTACACCGCATACCG |
| 2675 | GTAAGCCGGGTCTCGTGTCGCCGT | ACGGCGACACGAGACCCGGCTTAC |
| 2676 | GCGTAGTGCGAACGCCCCGACCTA | TAGGTCGGGGCGTTCGCACTACGC |
| 2677 | TCCTCGCGGCTTACGTCAAATTCG | CGAATTTGACGTAAGCCGCGAGGA |
| 2678 | CGACGTTCAAAGCGGGAGAGGAGG | CCTCCTCTCCCGCTTTGAACGTCG |
| 2679 | CGAGGCACCCCGACATGTTGAGAT | ATCTCAACATGTCGGGGTGCCTCG |
| 2680 | CTATTTCGTGCCGCGTCGGACAAG | CTTGTCCGACGCGGCACGAAATAG |
| 2681 | GGCTGCTCAGTGACGTGTCAACTG | CAGTTGACACGTCACTGAGCAGCC |
| 2682 | ATCACTCGTGCGTACCCGACCGTC | GACGGTCGGGTACGCACGAGTGAT |
| 2683 | CGAGATGTCCTATACCGTGGCGAA | TTCGCCACGGTATAGGACATCTCG |
| 2684 | TCACACCGAGCCCCATAAATGAAA | TTTCATTTATGGGGCTCGGTGTGA |
| 2685 | AGCTACGTGTCTCGAGCAAAAGCG | CGCTTTTGCTCGAGACACGTAGCT |
| 2686 | TCAGGGCGAGTTTTTTCAGCGGCG | CGCCGCTGAAAAAACTCGCCCTGA |
| 2687 | TTCGTTCTGTCTATTTTTGCCCCG | CGGGGCAAAAATAGACAGAACGAA |
| 2688 | TGGTATGCCCAGGATCCAGCCTAC | GTAGGCTGGATCCTGGGCATACCA |
| 2689 | TCTCAGTCGTTAGGCCAATGGCGG | CCGCCATTGGCCTAACGACTGAGA |
| 2690 | AAAGATCACCGTGGAGCGATCGGC | GCCGATCGCTCCACGGTGATCTTT |
| 2691 | TAGCAGGACTTGCACTCGTGATGC | GCATCACGAGTGCAAGTCCTGCTA |
| 2692 | TGCCCACGGTACCGTTCAAGGCTG | CAGCCTTGAACGGTACCGTGGGCA |
| 2693 | TGAGGTGCGTCGCCCTAAGTAATG | CATTACTTAGGGCGACGCACCTCA |
| 2694 | AGCAAGGGTTACAACCCGCAACCC | GGGTTGCGGGTTGTAACCCTTGCT |
| 2695 | CACAACAGCCAGTATTCGCCACAA | TTGTGGCGAATACTGGCTGTTGTG |
| 2696 | GGCAACACCATACTCGACGAGCTC | GAGCTCGTCGAGTATGGTGTTGCC |
| 2697 | GGCTGGATTGACAATTTAGCCCCT | AGGGGCTAAATTGTCAATCCAGCC |
| 2698 | CGTGAGAAATGCTACACGCGTCAG | CTGACGCGTGTAGCATTTCTCACG |
| 2699 | CGCATCTGCCCCATTTTGTTCCTT | AAGGAACAAAATGGGGCAGATGCG |
| 2700 | GTCGGCCTAGTCGGCAGAACGGTG | CACCGTTCTGCCGACTAGGCCGAC |
| 2701 | TCCCTCACCTTCCAAAAATGTGCT | AGCACATTTTTGGAAGGTGAGGGA |
| 2702 | GGGCAAGAACATGAGAACAGACCG | CGGTCTGTTCTCATGTTCTTGCCC |
| 2703 | TCGTCCTGGTACGACTTGCGTAGA | TCTACGCAAGTCGTACCAGGACGA |
| 2704 | TGGCGGTTGCATGTGATGATCAAG | CTTGATCATCACATGCAACCGCCA |
| 2705 | CCTCGCGTGAGTAAAAACCGTCCG | CGGACGGTTTTTACTCACGCGAGG |
| 2706 | ACTTCCGCCACAGAATGCGGCCAG | CTGGCCGCATTCTGTGGCGGAAGT |
| 2707 | GTGTAGAGCTTGGGTAGCCCCGTT | AACGGGGCTACCCAAGCTCTACAC |
| 2708 | CGCAGCATCCGAGTTAACACACAT | ATGTGTGTTAACTCGGATGCTGCG |
| 2709 | ATGAGCCTGGGATGATCCGCTGGT | ACCAGCGGATCATCCCAGGCTCAT |
| 2710 | CCTGGCATAAGTGCCGACATGCTT | AAGCATGTCGGCACTTATGCCAGG |
| 2711 | GCGCATGAAAAACTACGACGGACG | CGTCCGTCGTAGTTTTTCATGCGC |
| 2712 | AAAGATGGGTCGATGGGAGCGTCT | AGACGCTCCCATCGACCCATCTTT |
| 2713 | ATCCTGGGCACGAGCGGATTTATC | GATAAATCCGCTCGTGCCCAGGAT |
| 2714 | TCACCGCATTTGATAGTTACGCGA | TCGCGTAACTATCAAATGCGGTGA |
| 2715 | TGGTGGAGCGGACTCTGGTGTTAT | ATAACACCAGAGTCCGCTCCACCA |
| 2716 | CACAATGAAAAAACAATGGCCCCA | TGGGGCCATTGTTTTTTCATTGTG |
| 2717 | CCTTGCCGCGCTTGTGGTACCAAC | GTTGGTACCACAAGCGCGGCAAGG |
| 2718 | CCGAGACCTTTGCCACACGAAAGA | TCTTTCGTGTGGCAAAGGTCTCGG |
| 2719 | ACCGCGGTGTACACCTGAGCAGGC | GCCTGCTCAGGTGTACACCGCGGT |
| 2720 | GTCGTACGCTTACCGCAGCGGAGA | TCTCCGCTGCGGTAAGCGTACGAC |
| 2721 | TCGTAATTTGACCGACACACGCAG | CTGCGTGTGTCGGTCAAATTACGA |
| 2722 | CCTAGACGGATACCCTGAGCGGAA | TTCCGCTCAGGGTATCCGTCTAGG |
| 2723 | AAGCGACAGCAGAGGTTCAGTCGC | GCGACTGAACCTCTGCTGTCGCTT |
| 2724 | GCGTGGACGATATCACCTGGGCGT | ACGCCCAGGTGATATCGTCCACGC |
| 2725 | GTCGGAGAGCCAGTGGTACGGCTT | AAGCCGTACCACTGGCTCTCCGAC |
| 2726 | TATCCGCACGGTATAGCAGTTGCA | TGCAACTGCTATACCGTGCGGATA |
| 2727 | CATCAGTCGGGCTACCTTCAGCCT | AGGCTGAAGGTAGCCCGACTGATG |
| 2728 | CGGATTAATGCCTTTCCTCGGAAT | ATTCCGAGGAAAGGCATTAATCCG |
| 2729 | TTCGTCGTGCCAAGCTAATGCAAG | CTTGCATTAGCTTGGCACGACGAA |
| 2730 | GGCCGAGACCACCAGTAACAGGTT | AACCTGTTACTGGTGGTCTCGGCC |
| 2731 | CGCGCGGAAGCATTGAAGTTACTA | TAGTAACTTGAATGCTTCCGCGCG |
| 2732 | TCGGCTTACCGCTTCGTCTGACTT | AAGTCAGACGAAGCGGTAAGCCGA |
| 2733 | GACTGACGTCAAGGCAAGCAACAC | GTGTTGCTTGCCTTGACGTCAGTC |
| 2734 | AGAGGAAGGAGGGGCTGTGACAGA | TCTGTCACAGCCCCTCCTTCCTCT |
| 2735 | TTCCAATGCGAGAGATGGCAGGCT | AGCCTGCCATCTCTCGCATTGGAA |
| 2736 | AAATGGGGTGCTTCGAATATGTCG | CGACATATTCGAAGCACCCCATTT |
| 2737 | GCTGTCGGATTATTGCACGCCTGT | ACAGGCGTGCAATAATCCGACAGC |
| 2738 | CCGACTTTGTTTATGTTGCTGGCG | CGCCAGCAACATAAACAAAGTCGG |
| 2739 | GCTGCGATATAACCCGTCCCAGAA | TTCTGGGACGGGTTATATCGCAGC |
| 2740 | TGAGCTGGGCGTCAACTCCGAAGA | TCTTCGGAGTTGACGCCCAGCTCA |
| 2741 | CCCAAGCATCCTAAATCTCCCTCG | CGAGGGAGATTTAGGATGCTTGGG |
| 2742 | CGACAGCAATCCACATGCATTCTT | AAGAATGCATGTGGATTGCTGTCG |
| 2743 | TGAATGGTCGGGAAACCAATGCAT | ATGCATTGGTTTCCCGACCATTCA |
| 2744 | CTTTGCATCGAGATGGGGGGTAGC | GCTACCCCGCATCTCGATGCAAAG |
| 2745 | TCCATTTCCTCCGCAACTCTCAGG | CCTGAGAGTTGCGGAGGAAATGGA |
| 2746 | CCACTACGCCATCCTGACAACGAG | CTCGTTGTCAGGATGGCGTAGTGG |
| 2747 | TAGTAAGGCCAATGTACGCCGTCC | GGACGGCGTACATTGGCCTTACTA |
| 2748 | GTCATGCATATGGGGCCTGTTTTC | GAAAACAGGCCCCATATGCATGAC |
| 2749 | ACCGGTAGACGTTAGCGGGTTCAA | TTGAACCCGCTAACGTCTACCGGT |
| 2750 | TTGGTTCAAACGGCCACACGTCTC | GAGACGTGTGGCCGTTTGAACCAA |
| 2751 | GACACAAACTGCAAGGGAGGCATG | CATGCCTCCCTTGCAGTTTGTGTC |
| 2752 | CTCGAGCGCTGTCATCATATCGGC | GCCGATATGATGACAGCGCTCGAG |
| 2753 | GCGGCTAAGGCACAAGTAGACGTG | CACGTCTACTTGTGCCTTAGCCGC |
| 2754 | ACAGCCTAAATGGCGCAAGACCGA | TCGGTCTTGCGCCNTTTAGGCTGT |
| 2755 | CCGATGATGTAAGCCGTCGGCCCT | AGGGCCGACGGCTTACATCATCGG |
| 2756 | AGGAGCAAACAAACGCCAGTGACA | TGTCACTGGCGTTTGTTTGCTCCT |
| 2757 | ACGAATTGGGTAGCCGGACTGAGA | TCTCAGTCCGGCTACCCAATTCGT |
| 2758 | CTGTTCCAGTTCGGCAAGTGCGGC | GCCGCACTTGCCGAACTGGAACAG |
| 2759 | AGACAAGTCAGGAACGCGTTTCCG | CGGAAACGCGTTCCTGACTTGTCT |
| 2760 | AGACGACGGCCAGATACGCTGCCA | TGGCAGCGTATCTGGCCGTCGTCT |
| 2761 | AGGAAGCGCTTCTTCCGGTTCTTC | GAAGAACCGGAAGAAGCGCTTCCT |
| 2762 | GATGGACGCAAACACAAGGCGATC | GATCGCCTTGTGTTTGCGTCCATC |
| 2763 | CGCATAGCAGTCTCCGCATCTTGG | CCAAGATGCGGAGACTGCTATGCG |
| 2764 | TGGTTCCGGTGTGCAACAGATAAA | TTTATCTGTTGCACACCGGAACCA |
| 2765 | CCGTATGCCACCTCCAGAACTCAA | TTGAGTTCTGGAGGTGGCATACGG |
| 2766 | GTAAAGGAACCCCTCGGGAATCCT | AGGATTCCCGAGGGGTTCCTTTAC |
| 2767 | GCCTGATGCTCGTTAAAATTGCGT | ACGCAATTTTAACGAGCATCAGGC |
| 2768 | TCGCACTTGGACCATGAGATCTGA | TCAGATCTCATGGTCCAAGTGCGA |
| 2769 | TTCTCAGGCTGGGCAAGAGTCTGT | ACAGACTCTTGCCCAGCCTGAGAA |
| 2770 | CGGACCTGGGGATGCTGGGATTAC | GTAATCCCAGCATCCCCAGGTCCG |
| 2771 | TCGAGCCGATAGGGTTGGCATTGC | GCAATGCCAACCCTATCGGCTCGA |
| 2772 | TACGTGTGTCCCACACACGTCGTA | TACGACGTGTGTGGGACACACGTA |
| 2773 | TGTGAAATTCGCGTTTCGCATCTT | AAGATGCGAAACGCGAATTTCACA |
| 2774 | TTGCAATGCTCCAAAAAAACTGCC | GGCAGTTTTTTTGGAGCATTGCAA |
| 2775 | TCTCATCATGGCTGTGGCTTTGAC | GTCAAAGCCACAGCCATGATGAGA |
| 2776 | ATTACACCGCTTGGTTTGGAGTGG | CCACTCCAAACCAAGCGGTGTAAT |
| 2777 | GCCGTGCAATGCACAGAGTTCAAG | CTTGAACTCTGTGCATTGCACGGC |
| 2778 | GAGATCAGACCGTGTCGGATGCTG | CAGCATCCGACACGGTCTGATCTC |
| 2779 | CCACCTATCTTGATGCGACCTGGA | TCCAGGTCGCATCAAGATAGGTGG |
| 2780 | CCGATCGCCGTTTATGTCTACGGC | GCCGTAGACATAAACGGCGATCGG |
| 2781 | GAAAATCACGGTAAGGCACGTTCG | CGAACGTGCCTTACCGTGATTTTC |
| 2782 | GATTCTCGCTTCCCAACGAGCATA | TATGCTCGTTGGGAAGCGAGAATC |
| 2783 | TGTGAAATGTGGCAGTCTCAGGGA | TCCCTGAGACTGCCACATTTCACA |
| 2784 | CGATCCTGCGTGCCTCATCCAGGC | GCCTGGATGAGGCACGCAGGATCG |
| 2785 | CCCTCAAGTGGGCGAGGGTTTTCA | TGAAAACCCTCGCCCACTTGAGGG |
| 2786 | TCGCCTCCGCCTCGTGTGTAGAAG | CTTCTACACACGAGGCGGAGGCGA |
| 2787 | TTCGCTTTCAGCTCATTGGAACGA | TCGTTCCAATGAGCTGAAAGCGAA |
| 2788 | TGTAATCTGAACAAGCGGACCCCT | AGGGGTCCGCTTGTTCAGATTACA |
| 2789 | TGGAATCTTTCTTGAGCGCCGTGA | TCACGGCGCTCAAGAAAGATTCCA |
| 2790 | GGCTTTCATCTTTAACCGCTCGGT | ACCGAGCGGTTAAAGATGAAAGCC |
| 2791 | TGATCCGAGCCATTCCTAATCACC | GGTGATTAGGAATGGCTCGGATCA |
| 2792 | TGGTAGGCGTGATGTCCTACGCAA | TTGCGTAGGACATCACGCCTACCA |
| 2793 | AGGCATCGGTAAGAAGGCCCTATG | CATAGGGCCTTCTTACCGATGCCT |
| 2794 | CGCCGCGAGACGATCCTTATTATT | AATAATAAGGATCGTCTCGCGGCG |
| 2795 | ACATGGACGAAATTACGCCCGTCA | TGACGGGCGTAATTTCGTCCATGT |
| 2796 | ACAGAAAGGTGGGGAGCCTAGCGT | ACGCTAGGCTCCCCACCTTTCTGT |
| 2797 | AGGCTTGCGAACATGGGTAGTGAC | GTCACTACCCATGTTCGCAAGCCT |
| 2798 | GCGTGGGCCTTGCTCCTGTTTAAC | GTTAAACAGGAGCAAGGCCCACGC |
| 2799 | GAATACAGAGCGTCCGATGTGCCC | GGGCACATCGGACGCTCTGTATTC |
| 2800 | GCGACTCTGTAGGGAGCGCGATAT | ATATCGCGCTCCCTACAGAGTCGC |
| 2801 | GGTGCACTCATATGCGTCGCATCG | CGATGCGACGCATATGAGTGCACC |
| 2802 | CTGTCCCACGGGGAAACCTTACTT | AAGTAAGGTTTCCCCGTGGGACAG |
| 2803 | TGGCTTACTGTCGCAATCTAGGCC | GGCCTAGATTGCGACAGTAAGCCA |
| 2804 | GCACTCAGTTTCCGGTATCCCATG | CATGGGATACCGGAAACTGAGTGC |
| 2805 | GTGAGGTTCACGTAAGGCACAGCG | CGCTGTGCCTTACGTGAACCTCAC |
| 2806 | GTAACGCCTTTGTCCCCAGCGTAT | ATACGCTGGGGACAAAGGCGTTAC |
| 2807 | GCATTGATATGGTCGGTCTCGCCT | AGGCGAGACCGACCATATCAATGC |
| 2808 | GTGGGTTTAAGTGACAACGGACGC | GCGTCCGTTGTCACTTAAACCCAC |
| 2809 | CAAAACCCTGCCGAAGATGTTGGT | ACCAACATCTTCGGCAGGGTTTTG |
| 2810 | TCCGAGGAGACTGAACCTGCTACC | GGTAGCAGGTTCAGTCTCCTCGGA |
| 2811 | CGGGGAAGAACGGATTCGCTAAAT | ATTTAGCGAATCCGTTCTTCCCCG |
| 2812 | TGGTTAGCTTATGTCGGAGCCACC | GGTGGCTCCGACATAAGCTAACCA |
| 2813 | ACGCGTCGATGAACTAAGGCTCGC | GCGAGCCTTAGTTCATCGACGCGT |
| 2814 | TTCTCCTGACGAGTACGCAGTGGG | CCCACTGCGTACTCGTCAGGAGAA |
| 2815 | TCCGCGGTTGCCGGTTTGTTAGGA | TCCTAACAAACCGGCAACCGCGGA |
| 2816 | TGGCGCATCTTTCAGGGGATGATG | CATCATCCCCTGAAAGATGCGCCA |
| 2817 | TCTTTGGTCCTTGGTGTTTACGCG | CGCGTAAACACCAAGGACCAAAGA |
| 2818 | GAGAACTCCCGCTACAAAGGAGCC | GGCTCCTTTGTAGCGGGAGTTCTC |
| 2819 | TTAACGTGGGAACCGTTGGTGAAT | ATTCACCAACGGTTCCCACGTTAA |
| 2820 | GGGACACCATCCTTGGGTTTGTTA | TAACAAACCCAAGGATGGTGTCCC |
| 2821 | CAACAAACCGCCTTGGGAAGTGAC | GTCACTTCCCAAGGCGGTTTGTTG |
| 2822 | TTGAAGGCCACCGATACTGATCGC | GCGATCAGTATCGGTGGCCTTCAA |
| 2823 | TCGTAATAGAACTGCGCCCAATGC | GCATTGGGCGCAGTTCTATTACGA |
| 2824 | GGCACGTTGCCCAAGTTGGATCCA | TGGATCCAACTTGGGCAACGTGCC |
| 2825 | ACATAGCTTGGCCGGACACCCACC | GGTGGGTGTCCGGCCAAGCTATGT |
| 2826 | CTTGCCGCCTTGCGAGTGGCTAAA | TTTAGCCACTCGCAAGGGGGCAAG |
| 2827 | AATGGCTCGCCAGATACCGCAGCC | GGCTGCGGTATCTGGCGAGCCATT |
| 2828 | CAAAAGGCGTGTCCGAACTTTTCA | TGAAAAGTTCGGACACGCCTTTTG |
| 2829 | CGTCCACTTAGGTGGAGATACGCC | GGCGTATCTCCACCTAAGTGGACG |
| 2830 | GAGCCTCTTCGTCCTGAAGACCGA | TCGGTCTTCAGGACGAAGAGGCTC |
| 2831 | AACATCAAGCGGCAATCTCCCTTC | GAAGGGAGATTGCCGCTTGATGTT |
| 2832 | CGTCCTGACATTATTAGCGCGTGC | GCACGCGCTAATAATGTCAGGACG |
| 2833 | TGTGCAGACCCTAACGACCTACGG | CCGTAGGTCGTTAGGGTCTGCACA |
| 2834 | TTAGGTCGGCCTAGACCCTCCGTA | TACGGAGGGTCTAGGCCGACCTAA |
| 2835 | TCACATCGCTTAACTGAGCGCATT | AATGCGCTCAGTTAAGCGATGTGA |
| 2836 | AGACCTTCCCACGCGAGATGCTAC | GTAGCATCTCGCGTGGGAAGGTCT |
| 2837 | TTCTTGCCAAAATGTGTCCAACCA | TGGTTGGACACATTTTGGCAAGAA |
| 2838 | CAGTTTTCATTGCAGCGAAAGCAA | TTGCTTTCGCTGCAATGAAAACTG |
| 2839 | GTGCCGATCCCGAGACAAGTTCCG | CGGAACTTGTCTCGGGATCGGCAC |
| 2840 | CATCCGGCCTCAGTGATTCTTACC | GGTAAGAATCACTGAGGCCGGATG |
| 2841 | TGCTGGAAGCCACAAACGTTACGT | ACGTAACGTTTGTGGCTTCCAGCA |
| 2842 | GAACGGCCAGGGGACAACTATCGT | ACGATAGTTGTCCCCTGGCCGTTC |
| 2843 | TCATCTAGGTCGAAGCGCAAGACA | TGTCTTGCGCTTCGACCTAGATGA |
| 2844 | TTTGGTTACCAGCACCCATGTTCC | GGAACATGGGTGCTGGTAACCAAA |
| 2845 | GACAACAGTCTGTCCGCCACATCC | GGATGTGGCGGACAGACTGTTGTC |
| 2846 | GCCAACAGGAGATGCTTGCACCAT | ATGGTGCAAGCATCTCCTGTTGGC |
| 2847 | CTAAGGACGCATTGACCCCTGAAC | GTTCAGGGGTCAATGCGTCCTTAG |
| 2848 | GGTCGCGTAGTGAGTCAGAGGCGT | ACGCCTCTGACTCACTACGCGACC |
| 2849 | TTACCTCATGAACCCTTCGCGGCG | CGCCGCGAAGGGTTCATGAGGTAA |
| 2850 | TATACAGCATCGTCGCCGGGCATA | TATGCCCGGCGACGATGCTGTATA |
| 2851 | GCTTAGTGGCGTCTTCGTCGTAGG | CCTACGACGAAGACGCCACTAAGC |
| 2852 | TGCACTCCGCAACCTTGTGAAATC | GATTTCACAAGGTTGCGGAGTGCA |
| 2853 | AACCCGTCATGCCGACTCCATCTA | TAGATGGAGTCGGCATGACGGGTT |
| 2854 | AGCACTAGTGGCGTGCGACTTTGC | GCAAAGTCGCACGCCACTAGTGCT |
| 2855 | TAAAAAGTGCCGCTAACCACGGAG | CTCCGTGGTTAGCGGCACTTTTTA |
| 2856 | CGCGGAATATTTGTCGTCCGATTC | GAATCGGACGACAAATATTCCGCG |
| 2857 | TTCTGCTATGCGTATGGGGGCCCG | CGGGCCCCCATACGCATAGCAGAA |
| 2858 | CGAACTACTGCGTCAGCCTCTCCC | GGGAGAGGCTGACGCAGTAGTTCG |
| 2859 | AGATGACGAATTAGCGGGGTTGGG | CCCAACCCCGCTAATTCGTCATCT |
| 2860 | AATAACAGTGGCAATGAGCGGGAA | TTCCCGCTCATTGCCACTGTTATT |
| 2861 | ATATGTTGATTCCCGTGCTGCACA | TGTGCAGCACGGGAATCAACATAT |
| 2862 | AGAGTGGGCACCACCAGGCAGACA | TGTCTGCCTGGTGGTGCCCACTCT |
| 2863 | AGGCCTGGGTTTCTGCGTCTTAGT | ACTAAGACGCAGAAACCCAGGCCT |
| 2864 | CGGACGTGACAAACGGACATACCC | GGGTATGTCCGTTTGTCACGTCCG |
| 2865 | CAAGTGTTTCGGCCCAACTCTCGA | TCGAGAGTTGGGCCGAAACACTTG |
| 2866 | GAACCCTTATCGGGATAGGCCCAA | TTGGGCCTATCCCGATAAGGGTTC |
| 2867 | CAGGACGATACCAAGCAGAACGCC | GGCGTTCTGCTTGGTATCGTCCTG |
| 2868 | GCGTCTTGTGATTCTGCCCTAACC | GGTTAGGGCAGAATCACAAGACGC |
| 2869 | AAACAACCATCAATGTCGGGTCCA | TGGACCCGACATTGATGGTTGTTT |
| 2870 | TGTAAAGACCAGTTGGCGGCTCTC | GAGAGCCGCCAACTGGTCTTTACA |
| 2871 | GCGTTTTGACTCGGTGGTCAGTCC | GGACTGACCACCGAGTCAAAACGC |
| 2872 | TGTATGGAGGCACGGCAAAGTCTT | AAGACTTTGCCGTGCCTCCATACA |
| 2873 | TTACCTAGGTTCCCGCTGACACGC | GCGTGTCAGCGGGAACCTAGGTAA |
| 2874 | CGGCTCGTGGGAATCCTCTGAAGA | TCTTCAGAGGATTCCCACGAGCCG |
| 2875 | CCGGCTCGGGCATTTCTTGGACCT | AGGTCCAAGAAATGCCCGAGCCGG |
| 2876 | CAACGATGGAATTGTCTCCTTGGG | CCCAAGGAGACAATTCCATCGTTG |
| 2877 | CGGGCTATTATCGGGATTATGGGG | CCCCATAATCCCGATAATAGCCCG |
| 2878 | ACGTACCTGAAGATGCAACGGCGG | CCGCCGTTGCATCTTCAGGTACGT |
| 2879 | CATGGTGCAGCACGCACAAGTAAC | GTTACTTGTGCGTGCTGCACCATG |
| 2880 | CGTCGATATGTCGGGCTATTGCCT | AGGCAATAGCCCGACATATCGACG |
| 2881 | AAATGCAGGGTTAAGAGGAGGCCC | GGGCCTCCTCTTAACCCTGCATTT |
| 2882 | TGCAAGGACTGATTCTCCCGCTGT | ACAGCGGGAGAATCAGTCCTTGCA |
| 2883 | GTTTTCGGAACGCCGCAGAGTTCA | TGAACTCTGCGGCGTTCCGAAAAC |
| 2884 | CCCTCGATGGTTCATTGGGAAGAC | GTCTTCCCAATGAACCATCGAGGG |
| 2885 | CCTGTTCGCTCATAATGGTGGGGT | ACCCCACCATTATGAGCGAACAGG |
| 2886 | GAAAGAACGATCGCGGAATAGCTG | CAGCTATTCCGCGATCGTTCTTTC |
| 2887 | TCCACCTGTGTGCCTTTATCCTCA | TGAGGATAAAGGCACACAGGTGGA |
| 2888 | TCCTCCGTGAACCGCTGTAGCGCA | TGCGCTACAGCGGTTCACGGAGGA |
| 2889 | TTGAGATTTTTACGGTTTCCCCGC | GCGGGGAAACCGTAAAAATCTCAA |
| 2890 | CGATAGGACGTGGGCATGTCCCAG | CTGGGACATGCCCACGTGCTATCG |
| 2891 | CCCGAACTTTGAGATCCGAGAACA | TGTTCTCGGATCTCAAAGTTCGGG |
| 2892 | TCACGCAGCTAGAGTCGCGTTACC | GGTAACGCGACTCTAGCTGCGTGA |
| 2893 | AGATAACGCCCACTGACGACATGC | GCATGTCGTCAGTGGGCGTTATCT |
| 2894 | ACGCTTAGAGCTCCGATGCCGAAT | ATTCGGCATCGGAGCTCTAAGCGT |
| 2895 | GGGCGATAACTTAAATTGTGCCGC | GCGGCACAATTTAAGTTATCGCCC |
| 2896 | AGGACGTTCATGCGTCTCTTTGCA | TGCAAAGAGACGCATGAACGTCCT |
| 2897 | CGGCTGGTAGAACTGTGCATCGTA | TACGATGCACAGTTCTACCAGCCG |
| 2898 | TTCGAAATGTACTTCCCACGCGGA | TCCGCGTGGGAAGTACATTTCGAA |
| 2899 | GCAGGTTGGCTGTCTTGTGGAGTC | GACTCCACAAGACAGCCAACCTGC |
| 2900 | CGTTTGGTTGCTTCAAGAACCGGT | ACCGGTTCTTGAAGCAACCAAACG |
| 2901 | CATACTTGGTTGTTGTGCCCACGC | GCGTGGGCACAACAACCAAGTATG |
| 2902 | GGGGTCGGCTGAAGTGTTTTATCC | GGATAAAACACTTCAGCCGACCCC |
| 2903 | GTGACGGTTGATTAACGACCGTGG | CCACGGTCGTTAATCAACCGTCAC |
| 2904 | CTTATGGCAGCGCCAGGGGCACTC | GAGTGCCCCTGGCGCTGCCATAAG |
| 2905 | GTTAGGGGACCCACCTCGTTTGAT | ATCAAACGAGGTGGGTCCCCTAAC |
| 2906 | CAATATAAATGCCGCGCATCGAGT | ACTCGATGCGCGGCATTTATATTG |
| 2907 | TTCTTCATCAGCAGTCCCCGAGAA | TTCTCGGGGACTGCTGATGAAGAA |
| 2908 | AGTTGCGTCCCTTGATGGCATTTT | AAAATGCCATCAAGGGACGCAACT |
| 2909 | CCGACTTTCGTCCACGATTCCTCT | AGAGGAATCGTGGACGAAAGTCGG |
| 2910 | ACTTGGCCGGACGACAGCAAAGAC | GTCTTTGCTGTCGTCCGGCCAAGT |
| 2911 | CACCGCGGTAGATGTATCCCTTCC | GGAAGGGATACATCTACCGCGGTG |
| 2912 | GTTAGCTTTAGCTCGGCACGCCTG | CAGGCGTGCCGAGCTAAAGCTAAC |
| 2913 | GCGCATAAGAAGGTCCGCTAAAGC | GCTTTAGCGGACCTTCTTATGCGC |
| 2914 | ACATCATCACGCCTGGCGTGACCA | TGGTCACGCCAGGCGTGATGATGT |
| 2915 | CCGGCGAAGTTTGGTGTGATTAGA | TCTAATCACACCAAACTTCGCCGG |
| 2916 | TGCACCGCCAGATTGTGCTGAGTC | GACTCAGCACAATCTGGCGGTGCA |
| 2917 | ACATGTGAAGTGAGTGCCGTCCAA | TTGGACGGCACTCACTTCACATGT |
| 2918 | CCTCTGGAGGGGATTAGCCACGCT | AGCGTGGCTAATCCCCTCCAGAGG |
| 2919 | CAATAGCCATGTCACTGGCAACGG | CCGTTGCCAGTGACATGGCTATTG |
| 2920 | ACCCATGGTTCCAACGTTCTTTCG | CGAAAGAACGTTGGAACCATGGGT |
| 2921 | AATCTGGTCTTGGCATCCTCCAAA | TTTGGAGGATGCCAAGACCAGATT |
| 2922 | GTATACCGGTGCATGCTGAAGCAA | TTGCTTCAGCATGCACCGGTATAC |
| 2923 | AGTGTTCTGGTTCGAGTCGACCCG | CGGGTCGACTCGAACCAGAACACT |
| 2924 | CGGGTATTCGACACACACGAGGAC | GTCCTCGTGTGTGTCGAATACCCG |
| 2925 | AGTGCAACAGAGCGCTTGGTCACG | CGTGACCAAGCGCTCTGTTGCACT |
| 2926 | TGCACCTATAGTTTGGTGCCGGTG | CACCGGCACCAAACTATAGGTGCA |
| 2927 | TGCTCACGTACCAGGACACTCGAG | CTCGAGTGTCCTGGTACGTGAGCA |
| 2928 | AGTCCACACCTCGAACGACAGGCG | CGCCTGTCGTTCGAGGTGTGGACT |
| 2929 | CGCCGACCTGGTCAAAGAGCGCTA | TAGCGCTCTTTGACCAGGTCGGCG |
| 2930 | GCCTAAGGGCCTGTCGTTTTCCGA | TCGGAAAACGACAGGCCCTTAGGC |
| 2931 | TGTGCGTGCTTATGTTCCGGTCTC | GAGACCGGAACATAAGCACGCACA |
| 2932 | CAACCGTTGGCCGTAACAAAAATC | GATTTTTGTTACGGCCAACGGTTG |
| 2933 | CGAGAATCAAGGCGTACCATCTCG | CGAGATGGTACGCCTTGATTCTCG |
| 2934 | GCGTAGGCAGCCTCCAGGGAATGG | CCATTCCCTGGAGGCTGCCTACGC |
| 2935 | GATGGTGTTTTCGCCAAGACCAAT | ATTGGTCTTGGCGAAAACACCATC |
| 2936 | CAAGCTAGGGACAGAATTGCCCAC | GTGGGCAATTCTGTCCCTAGCTTG |
| 2937 | TAAATAGGCGAAACCGTTCGTGGC | GCCACGAACGGTTTCGCCTATTTA |
| 2938 | TCAAGACCCGCAATGTGTTCATGT | ACATGAACACATTGCGGGTCTTGA |
| 2939 | GCGGCTGGTAGACTCTTTGCACAA | TTGTGCAAAGAGTCTACCAGCCGC |
| 2940 | CAGGCGTAAACCTGAACCAAACGG | CCGTTTGGTTCAGGTTTACGCCTG |
| 2941 | GCCGATCTGTGCTGAGGTTCATCA | TGATGAACCTCAGCACAGATCGGC |
| 2942 | GATATCGCGTCGCAATATCACGCG | CGCGTGATATTGCGACGCGATATC |
| 2943 | CCCTGCACGATTAAGCCACCTGTA | TACAGGTGGCTTAATCGTGCAGGG |
| 2944 | TGACATACAGATTTGTGTGGCCCC | GGGGCCACACAAATCTGTATGTCA |
| 2945 | GTTTGCGGCCGGTATTCACGATGT | ACATCGTGAATACCGGCCGCAAAC |
| 2946 | TTTTACCTGGCCATTGGTGAGCTC | GAGCTCACCAATGGCCAGGTAAAA |
| 2947 | CTCTACTCAATCAGGGTGGGAGCG | CGCTCCCACCCTGATTGAGTAGAG |
| 2948 | GGGTTGGAGGGAGTCTTGACCATT | AATGGTCAAGACTCCCTCCAACCC |
| 2949 | CGAGGTCGGTAAGGAAAAGCTTGC | GCAAGCTTTTCCTTACCGACCTCG |
| 2950 | CTTTACGCAGGCACCTCCGAGCTG | CAGCTCGGAGGTGCCTGCGTAAAG |
| 2951 | CATTGTATGGCCACGTGATTGACG | CGTCAATCACGTGGCCATACAATG |
| 2952 | GTACGGTGCGAGAGCGCCTAAGCG | CGCTTAGGCGCTCTCGCACCGTAC |
| 2953 | TTCCATATGCCGAAATGGACACAA | TTGTGTCCATTTCGGCATATGGAA |
| 2954 | TACGCCTTCCGCTATAGCTCGTGA | TCACGAGCTATAGCGGAAGGCGTA |
| 2955 | CTGTACGCCACGCATGAAGGGTGA | TCACCCTTCATGCGTGGCGTACAG |
| 2956 | CTTACGCGTCCAATGACTGCCACC | GGTGGCAGTCATTGGACGCGTAAG |
| 2957 | CACATGGTAGAACTCGATCGGCAG | CTGCCGATCGAGTTCTACCATGTG |
| 2958 | CGCACCGGAAACTAGTGGATGTGT | ACACATCCACTAGTTTCCGGTGCG |
| 2959 | ACTATGGCAACCGACACTTGGTCC | GGACCAAGTGTCGGTTGCCATAGT |
| 2960 | CTAGTTTGCGCTACCCACCTGCAA | TTGCAGGTGGGTAGCGCAAACTAG |
| 2961 | TAGTATCGCCCGACAATAGCCTGG | CCAGGCTATTGTCGGGCGATACTA |
| 2962 | CCAATATTTACGGCCTGATCAGCG | CGCTGATCAGGCCGTAAATATTGG |
| 2963 | ATGGCTATCCCTTACTGGCTCGCC | GGCGAGCCAGTAAGGGATAGCCAT |
| 2964 | CAAAACTTGGCAGGCTTGGGACTT | AAGTCCCAAGCCTGCCAAGTTTTG |
| 2965 | AATGACCGAGGCTGCAAGATTGAC | GTCAATCTTGCAGCCTCGGTCATT |
| 2966 | ATCATCTTTCGCCACCAGACATGG | CCATGTCTGGTGGCGAAAGATGAT |
| 2967 | CGTTATTACCGATGCACACGTTGC | GCAACGTGTGCATCGGTAATAACG |
| 2968 | CACACTGGCAATCGCCTCCCTCGT | ACGAGGGAGGCGATTGCCAGTGTG |
| 2969 | AGGTTGGTAGGAAATCGGAGCGCT | AGCGCTCCGATTTCCTACCAACCT |
| 2970 | GCTGAACCACTGTGGTCAAGATGC | GCATCTTGACCACAGTGGTTCAGC |
| 2971 | CGTTGAGTACGACACGGTCGAGGT | ACCTCGACCGTGTCGTACTCAACG |
| 2972 | TTTTTCCGCCGCAATGTGATCTAA | TTAGATCACATTGCGGCGGAAAAA |
| 2973 | ACAATACCTCGACCGCTCAGCATC | GATGCTGAGCGGTCGAGGTATTGT |
| 2974 | AGTATCCCTGCTGGCATACACGGG | CCCGTGTATGCCAGCAGGGATACT |
| 2975 | TCTTGGGCTCGGTAGTTCAGCACT | AGTGCTGAACTACCGAGCCCAAGA |
| 2976 | CCCTATATCGAGCCCATAGGGCGA | TCGCCCTATGGGCTCGATATAGGG |
| 2977 | CACGAGTGGCATCAACGGCCTACT | AGTAGGCCGTTGATGCCACTCGTG |
| 2978 | TGCAGGGTCCGATGTGTTCAAGTA | TACTTGAACACATCGGACCCTGCA |
| 2979 | GCTTGACCGCTGCTAACCTCGTAC | GTACGAGGTTAGCAGCGGTCAAGC |
| 2980 | TTTTGCATCTCTCCACCATCCAGA | TCTGGATGGTGGAGAGATGCAAAA |
| 2981 | AGAATGTGCACCGGCTTCCATCTT | AAGATGGAAGCCGGTGCACATTCT |
| 2982 | TGTTATGACCCGCTCTGTGGCGTG | CACGCCACAGAGCGGGTCATAACA |
| 2983 | GGAGCTCCTGTTTCATCGAGGCTA | TAGCCTCGATGAAACAGGAGCTCC |
| 2984 | CATTTTGCTGTTTGGGGGTCCCAT | ATGGGACCCCCAAACAGCAAAATG |
| 2985 | CCCGCTCCTTCACGTGAGACGAGA | TCTCGTCTCACGTGAAGGAGCGGG |
| 2986 | GCGCTCAAGTCGATTGCCACAACC | GGTTGTGGCAATCGACTTGAGCGC |
| 2987 | CGGTTGACGGAGACCGCAGTACTT | AAGTACTGCGGTCTCCGTCAACCG |
| 2988 | ACTCAAGACCGGTGCACCTCCAGC | GCTGGAGGTGCACCGGTCTTGAGT |
| 2989 | TTTCGTGTGCATGCAAGTAATGGC | GCCATTACTTGCATGCACACGAAA |
| 2990 | GCGGCGTTAGCTCGAGCTAACAAA | TTTGTTAGCTCGAGCTAACGCCGC |
| 2991 | GGGTATCCTGCCCGAGCAGTAATT | AATTACTGCTCGGGCAGGATACCC |
| 2992 | GGCTCCGAATCTCTTGTCCGGTCT | AGACCGGACAAGAGATTCGGAGCC |
| 2993 | AGGATGGCCACGCCGAATCAAAGT | ACTTTGATTCGGCGTGGCCATCCT |
| 2994 | GTGCGGGGACGTTTACATAACGAG | CTCGTTATGTAAACGTCCCCGCAC |
| 2995 | ACTTTTGACCTGAGGCCGCTTGCA | TGCAAGCGGCCTCAGGTCAAAAGT |
| 2996 | ACTCCGCTTCAATGGAGACCGTTG | CAACGGTCTCCATTGAAGCGGAGT |
| 2997 | GATCGGAATTCGCCGCCATATTGA | TCAATATGGCGGCGAATTCCGATC |
| 2998 | ATGCGTGCCCATGGAATGACTTTT | AAAAGTCATTCCATGGGCACGCAT |
| 2999 | CCGCATCGCACGAAGGCAGGTCAT | ATGACCTGCCTTCGTGCGATGCGG |
| 3000 | CACCCTATGCGTCTCCAATTCCTG | CAGGAATTGGAGACGCATAGGGTG |
| 3001 | TGATATGCATCGCTGAGCCTCTGT | ACAGAGGCTCAGCGATGCATATCA |
| 3002 | AGCTTCACACGCTCACTGAACCTG | CAGGTTCAGTGAGCGTGTGAAGCT |
| 3003 | AACCCGGAACCTCCTCTCACTCGG | CCGAGTGAGAGGAGGTTCCGGGTT |
| 3004 | CTCGTCAAACTTGGCCGAGGAGTC | GACTCCTCGGCCAAGTTTGACGAG |
| 3005 | GTAGCTGGCAACAGGCAATCAGGA | TCCTGATTGCCTGTTGCCAGCTAC |
| 3006 | CTTGTCACGAATATTCGCCAAGCG | CGCTTGGCGAATATTCGTGACAAT |
| 3007 | CAGTATCTGAAACACGGGGTGCTG | CAGCACCCCGTGTTTCAGATACTG |
| 3008 | GGCTAAAATGGGCGCCCACGTGTA | TACACGTGGGCGCCCATTTTAGCC |
| 3009 | ATGAGAGCCAAGCGCCTCAACTCC | GGAGTTGAGGCGCTTGGCTCTCAT |
| 3010 | TATTGTTAGGCACCGCTTCGCGCT | AGCGCGAAGCGGTGCCTAACAATA |
| 3011 | GGAACTAGATTGCCAGTGCTCGCC | GGCGAGCACTGGCAATCTAGTTCC |
| 3012 | AGTCGACCCCAAGGCAACTGGGTC | GACCCAGTTGCCTTGGGGTCGACT |
| 3013 | GGTACTGTTAGCTCGACGATGGCC | GGCCATCGTCGAGCTAACAGTACC |
| 3014 | CCGCAATACTTGACGGTAACAGGG | CCCTGTTACCGTCAAGTATTGCGG |
| 3015 | AATTCCGGGTTTGAACGGTTGGAA | TTCCAACCGTTCAAACCCGGAATT |
| 3016 | GACACGCAATCGGGTCTATGCGAA | TTCGCATAGACCCGATTGCGTGTC |
| 3017 | GATTTTGGCGTCTCATTGCGTGAT | ATCACGCAATGAGACGCCAAAATC |
| 3018 | TGCCATAGGGAGGAAACGCAATTA | TAATTGCGTTTCCTCCCTATGGCA |
| 3019 | GAGGTGCCCATGTTAGTGGTGTCC | GGACACCACTAACATGGGCACCTC |
| 3020 | GCTTTAGCGGTCATACGACCACCA | TGGTGGTCGTATGACCGCTAAAGC |
| 3021 | CCGCTACCAACAATCCGATTAACG | CGTTAATCGGATTGTTGGTAGCGG |
| 3022 | GAGGATCTGGCCACATCGAGAAAG | CTTTCTCGATGTGGCCAGATCCTC |
| 3023 | CTCGTTTGGTACCACGTTTTGCCG | CGGCAAAACGTGGTACCAAACGAG |
| 3024 | AATACACGCGGCGTAAACAGACGA | TCGTCTGTTTACGCCGCGTGTATT |
| 3025 | TGTCATGGGCCAAATGACAGTGGC | GCCACTGTCATTTGGCCCATGACA |
| 3026 | ACAGCACTTCCGACCCGTGTACGA | TCGTACACGGGTCGGAAGTGCTGT |
| 3027 | CTCCGTAAAGAGCACAGCTTTGCC | GGCAAAGCTGTGCTCTTTACGGAG |
| 3028 | ACGAACAGGTAGGGATCGGTCCTC | GAGGACCGATCCCTACCTGTTCGT |
| 3029 | TGGATCCACCTTACCGCGCCATCG | CGATGGCGCGGTAAGGTGGATCCA |
| 3030 | AGTATCAAATAGCGGCGCGGCAAG | CTTGCCGCGCCGCTATTTGATACT |
| 3031 | GAATTACATTGTGGATGGAGGCGG | CCGCCTCCATCCACAATGTAATTC |
| 3032 | CTCCTCGGGGAGTCGAGGAGTACG | CGTACTCCTCGACTCCCCGAGGAG |
| 3033 | AGTGTCGAGCCAACTCCCACCAAT | ATTGGTGGGAGTTGGCTCGACACT |
| 3034 | AAATGACATCCGTTTGGCCACAGC | GCTGTGGCCAAACGGATGTCATTT |
| 3035 | CGAATCATATCGCCATCGAACTGG | CCAGTTCGATGGCGATATGATTCG |
| 3036 | TATAATGCACTCGCTTGGTGCGCA | TGCGCACCAAGCGAGTGCATTATA |
| 3037 | GCCAAGCAGATGGTAATTATGGCG | CGCCATAATTACCATCTGCTTGGC |
| 3038 | CACGCGGGAAGAGCACGTAGAACT | AGTTCTACGTGCTCTTCCCGCGTG |
| 3039 | TACCCGAGAATTTGGAGAACAGCG | CGCTGTTCTCCAAATTCTCGGGTA |
| 3040 | TGACGGCAAACTGTGGCATCTATC | GATAGATGCCACAGTTTGCCGTCA |
| 3041 | CACAGTGTTCCAGCCCTTGACGAT | ATCGTCAAGGGCTGGAACACTGTG |
| 3042 | TACCCGCCCACACATGAAAGTTGG | CCAACTTTCATGTGTGGGCGGGTA |
| 3043 | TGGCATATTTAAGATTCGGCGACG | CGTCGCCGAATCTTAAATATGCCA |
| 3044 | ACTGAAAAAAGAACGGGTAGCGGG | CCCGCTACCCGTTCTTTTTTCAGT |
| 3045 | TCTGACCGCAATAGGTGGTCATTG | CAATGACCACCTATTGCGGTCAGA |
| 3046 | ACTTTTTGGCGGGCCCTCTCTCGT | ACGAGAGAGGGCCCGCCAAAAAGT |
| 3047 | CTGCCCAGATCATTGCGCGATCCG | CGGATCGCGCAATGATCTGGGCAG |
| 3048 | CGGAGGTTAAATGCTTTAACCGGC | GCCGGTTAAAGCATTTAACCTCCG |
| 3049 | AGGCGTCTCCAAACGTCCTTCTGT | ACAGAAGGACGTTTGGAGACGCCT |
| 3050 | AGATGCTATCCTGAGTGGGCCTGC | GCAGGCCCACTCAGGATAGCATCT |
| 3051 | ACAGGGTGAAGAGACCGTGGGATG | CATCCCACGGTCTCTTCACCCTGT |
| 3052 | GACTGTCTAACGGACGACACGACG | CGTCGTGTCGTCCGTTAGACAGTC |
| 3053 | AGCTGTTAGGACCCGACAACCGGT | ACCGGTTGTCGGGTCCTAACAGCT |
| 3054 | TTGCGTAGTGTGGGCATTTCCTCT | AGAGGAAATGCCCACACTACGCAA |
| 3055 | ATGCGCGCTTCTTTCCTTGATGTA | TACATCAAGGAAAGAAGCGCGCAT |
| 3056 | TTAAGGGCGTCCGCGTCTATTCAG | CTGAATAGACGCGGACGCCCTTAA |
| 3057 | ACCTTTAAACTTGTACCGCGGCCC | GGGCCGCGGTACAAGTTTAAAGGT |
| 3058 | AGGGATGCAGAGGCACCACATGTT | AACATGTGGTGCCTCTGCATCCCT |
| 3059 | CGGTTCGACGTATGAGCATCCGCA | TGCGGATGCTCATACGTCGAACCG |
| 3060 | CAGGGCGATAGTCACATGGAGGTT | AACCTCCATGTGACTATCGCCCTG |
| 3061 | GCTTGACTGCCCCGTTTCATATGT | ACATATGAAACGGGGCAGTCAAGC |
| 3062 | CGAAGGGGTTGTGCAATTACCCGA | TCGGGTAATTGCACAACCCCTTCG |
| 3063 | AAAACGCACCGCAATGACAAAATT | AATTTTGTCATTGCGGTGCGTTTT |
| 3064 | ATTCCTGGACAAGACCCTCAACCG | CGGTTGAGGGTCTTGTCCAGGAAT |
| 3065 | CCTACCTGCCTGCTAGCGGTGAGG | CCTCACCGCTAGCAGGCAGGTAGG |
| 3066 | GCTCGTAAATGGGGAGGAATTGGA | TCCAATTCCTCCCCATTTACGAGC |
| 3067 | ACATGAAAACAGGCTCAATTGGGG | CCCCAATTGAGCCTGTTTTCATGT |
| 3068 | GTTCCGCACATGGATTGAGGTCTC | GAGACCTCAATCCATGTGCGGAAC |
| 3069 | GGCACCCAATACCACGAAGAAGAA | TTCTTCTTCGTGGTATTGGGTGCC |
| 3070 | AGGGGCATTTCGAACTCCATCTTT | AAAGATGGAGTTCGAAATGCCCCT |
| 3071 | CATCATCACAAAGGAACGTCGGTG | CACCGACGTTCCTTTGTGATGATG |
| 3072 | TAAAGACCCACCGTCAGCAGCAGC | GCTGCTGCTGACGGTGGGTCTTTA |
| 3073 | CCCCAGGCGTAATGCACCACATAG | CTATGTGGTGCATTACGCCTGGGG |
| 3074 | GCAGGTCGAACGCTAGTGGTTGAA | TTCAACCACTAGCGTTCGACCTGC |
| 3075 | GGAACTTAGGAGTTCACGTCGCCA | TGGCGACGTGAACTCCTAAGTTCC |
| 3076 | GCAGATACGGCTAGCTGAGGTGGC | GCCACCTCAGCTAGCCGTATCTGC |
| 3077 | CACAGGCCTAGAGCCTCGGCGTTC | GAACGCCGAGGCTCTAGGCCTGTG |
| 3078 | GTTTTGCGCGCATGAGGTTCATTA | TAATGAACCTCATGCGCGCAAAAC |
| 3079 | TTGCGCCTGATGCCAGCAGTACTA | TAGTACTGCTGGCATCAGGCGCAA |
| 3080 | GATATCAGGCTTTCCCACTGCCGC | GCGGCAGTGGGAAAGCCTGATATC |
| 3081 | TGCGCGGAGACGGAGATCTATGAA | TTCATAGATCTCCGTCTCCGCGCA |
| 3082 | CATTGGTGTTGGCTGAGAGTGGAC | GTCCACTCTCAGCCAACACCAATG |
| 3083 | GTCGGCACTTGGGCACCATTAATA | TATTAATGGTGCCCAAGTGCCGAC |
| 3084 | ATCGATCGGTGTCTCACCACGGAG | CTCCGTGGTGAGACACCGATCGAT |
| 3085 | CGTAGCCTTCCACCGTGTCGATAG | CTATCGACACGGTGGAAGGCTACG |
| 3086 | CGCTCTCCGTCTGAGGAAAAGGGG | CCCCTTTTCCTCAGACGGAGAGCG |
| 3087 | TCGCCCCAGCCAAGGATATATTGC | GCAATATATCCTTGGCTGGGGCGA |
| 3088 | TCTCTTGCAAGGAACTCTGCCGTC | GACGGCAGAGTTCCTTGCAAGAGA |
| 3089 | GTCCTGGACAGACGGAGGGTGTTA | TAACACCCTCCGTCTGTCCAGGAC |
| 3090 | GCCAAATTAAGCGGGCTCGTAATC | GATTACGAGCCCGCTTAATTTGGC |
| 3091 | CCATTTGTTGACCGATGGGAGGGG | CCCCTCCCATCGGTCAACAAATGG |
| 3092 | TGGTCAAAAGAGCACGATCCAGGA | TCCTGGATCGTGCTCTTTTGACCA |
| 3093 | CGCTACTAAGACGCCCCTGTCCAC | GTGGACAGGGGCGTCTTAGTAGCG |
| 3094 | CATACCTCCCGCTTGGATTCACTG | CAGTGAATCCAAGCGGGAGGTATG |
| 3095 | CCGCGGAAGGAATGTCATCTACAA | TTGTAGATGACATTCCTTCCGCGG |
| 3096 | CACGGGACATTCATTCACAGGACG | CGTCCTGTGAATGAATGTCCCGTG |
| 3097 | AGGAGTCACCCACTCCGCACAAAA | TTTTGTGCGGAGTGGGTGACTCCT |
| 3098 | TCATGACAGCGCACCCCATACCAT | ATGGTATGGGGTGCGCTGTCATGA |
| 3099 | GGTAGGGGACTATCGATCGTGCTG | CAGCACGATCGATAGTCCCCTACC |
| 3100 | ATGTCTCACTACCGCACGTAGCGG | CCGCTACGTGCGGTAGTGAGACAT |
| 3101 | ACGGAGGAGCGACTCGTTCGCTGC | GCAGCGAACGAGTCGCTCCTCCGT |
| 3102 | GAAGTCTGTCGCCGGTGGACGGAC | GTCCGTCCACCGGCGACAGACTTC |
| 3103 | CCGTAACGTGTATTCGGACGAGCG | CGCTCGTCCGAATACACGTTACGG |
| 3104 | CGTGGAAGCGACTTAACCAATCGT | ACGATTGGTTAAGTCGCTTCCACG |
| 3105 | GGCATGGGCTATGCCTCACACTAG | CTAGTGTGAGGCATAGCCCATGCC |
| 3106 | GGGTCGTATTTCAGCATCGTTCGT | ACGAACGATGCTGAAATACGACCC |
| 3107 | AATGGTCGCGCAAACCGTAAGAAT | ATTCTTACGGTTTGCGCGACCATT |
| 3108 | CTGGATTCGGTACGTCCAACGTTT | AAACGTTGGACGTACCGAATCCAG |
| 3109 | CGCAAAAACACCCGTAGCCAAGAA | TTCTTGGCTACGGGTGTTTTTGCG |
| 3110 | TATGGATACGCTTTTGGACTGGGC | GCCCAGTCCAAAAGCGTATCCATA |
| 3111 | GCTTCAAACGCGCTTCACGCTGGT | ACCAGCGTGAAGCGCGTTTGAAGC |
| 3112 | TACAGCCCGCTCTACCTCGCCACC | GGTGGCGAGGTAGAGCGGGCTGTA |
| 3113 | TCAACCGATGTCAAAATGCACGTT | AACGTGCATTTTGACATCGGTTGA |
| 3114 | AGCTCTCTCCGAAGTAGGGCGGTA | TACCGCCCTACTTCGGAGAGAGCT |
| 3115 | ACGCACACATGGAGACTTGGCTCC | GGAGCCAAGTCTCCATGTGTGCGT |
| 3116 | TTCTTGAAAGCTAGTGGGGCGCTA | TAGCGCCCCACTAGCTTTCAAGAA |
| 3117 | CAATCACGGCTGGGCTATTCTGTG | CACAGAATAGCCCAGCCGTGATTG |
| 3118 | GTGGCGACCCGTCGGTGAAAGAGT | ACTCTTTCACCGACGGGTCGCCAC |
| 3119 | CGTCGAATGCCGAACCAGTTAAGT | ACTTAACTGGTTCGGCATTCGACG |
| 3120 | TGCGTATTTGCATGCTCACAGCTG | CAGCTGTGAGCATGCAAATACGCA |
| 3121 | CGCAGTTGGTTTGTGCACGGCTGC | GCAGCCGTGCACAAACCAACTGCG |
| 3122 | GTTTTTCCGTGAAAACTGGCATCG | CGATGCCAGTTTTCACGGAAAAAC |
| 3123 | ACAGGTTCCTCCACCACGATTTGA | TCAAATCGTGGTGGAGGAACCTGT |
| 3124 | CTAGCGCGCTTTTAGGTCCTTGCG | CGCAAGGACCTAAAAGCGCGCTAG |
| 3125 | CAAAATCAAAGGGATCAACCGGTG | CACCGGTTGATCCCTTTGATTTTG |
| 3126 | AACGTAACCCCAGTGAGTCAGGCA | TGCCTGACTCACTGGGGTTACGTT |
| 3127 | TCAACCGGTGCACTTTAGAACGCC | GGCGTTCTAAAGTGCACCGGTTGA |
| 3128 | ATCGCAAAGTTGCAGGCGAATACT | AGTATTCGCCTGCAACTTTGCGAT |
| 3129 | ATATGTCCCTGGGTGCTGCACAAC | GTTGTGCAGCACCCAGGGACATAT |
| 3130 | TGGCACTTTGTAGTGCTGCGGTGG | CCACCGCAGCACTACAAAGTGCCA |
| 3131 | ACGCACGACGTCCTTCTAAGCTCG | CGAGCTTAGAAGGACGTCGTGCGT |
| 3132 | CCCACGTGCACTATAGGGATTTCG | CGAAATCCCTATAGTGCACGTGGG |
| 3133 | CCGCGCTTGGTCAGTCATCCTTGC | GCAAGGATGACTGACCAAGCGCGG |
| 3134 | AGCGGCTCAGGGAATAACAACAGG | CCTGTTGTTATTCCCTGAGCCGCT |
| 3135 | ACAACGCGATCGGAGGCAACCAGT | ACTGGTTGCCTCCGATCGCGTTGT |
| 3136 | AGCAATTGCCTCCGTAGAAACCCA | TGGGTTTCTACGGAGGCAATTGCT |
| 3137 | GAGTCGTGGCATCGCCTGCTATCG | CGATAGCAGGCGATGCCACGACTC |
| 3138 | TCTATGCAAATACTGCGCTTGCGA | TCGCAAGCGCAGTATTTGCATAGA |
| 3139 | TCAGCTTAAGTTACGGTGTGGCCG | CGGCCACACCGTAACTTAAGCTGA |
| 3140 | TCCAAGGTCGAACAGGGATCAGAA | TTCTGATCCCTGTTCGACCTTGGA |
| 3141 | GTTAGGCTGGCGTCAATAGCGCTT | AAGCGCTATTGACGCCAGCCTAAC |
| 3142 | GGTGTCATAAGGAAGAGGGCATCG | CGATGCCCTCTTCCTTATGACACC |
| 3143 | CCGGCGGGCTAGATCAATATTTCT | AGAAATATTGATCTAGCCCGCCGG |
| 3144 | CTAACGTCAAGTTTTACGCCCCGA | TCGGGGCGTAAAACTTGACGTTAG |
| 3145 | GCAGCACAGTTTTCCGATTTGCGG | CCGCAAATCGGAAAACTGTGCTGC |
| 3146 | CGCACGCAAGGGGAGGGATGACTG | CAGTCATCCCTCCCCTTGCGTGCG |
| 3147 | CGGGGCCGAAAAGGACGTCACAAG | CTTGTGACGTCCTTTTCGGCCCCG |
| 3148 | TTCTCCAACACGGCTAACCGGTAG | CTACCGGTTAGCCGTGTTGGAGAA |
| 3149 | TTACAGCCTGGCCCGAGGTAGTTG | CAACTACCTCGGGCCAGGCTGTAA |
| 3150 | TTTCGGGCAGCATGAGTTATCGAA | TTCGATAACTCATGCTGCCCGAAA |
| 3151 | CTACTGGACGCCCTGCTTCGAAGT | ACTTCGAAGCAGGGCGTCCAGTAG |
| 3152 | GGTCGTCCGACGTGAAAAGACCAA | TTGGTCTTTTCACGTCGGACGACC |
| 3153 | GTTTTCGAGCTCTTTCTCCGCAGG | CCTGCGGAGAAAGAGCTCGAAAAC |
| 3154 | GCGTGAAGGTACCCAGTGTCACAG | CTGTGACACTGGGTACCTTCACGC |
| 3155 | TTTCTGAACGCTTCGACGCAACAC | GTGTTGCGTCGAAGCGTTCAGAAA |
| 3156 | TGCTAATAAGCACGCCTAGCCCGT | ACGGGCTAGGCGTGCTTATTAGCA |
| 3157 | AAATTAATTGTGGTGGCTCCGGCG | CGCCGGAGCCACCACAATTAATTT |
| 3158 | TTACAATCCTCGGGCTCACTGACA | TGTCAGTGAGCCCGAGGATTGTAA |
| 3159 | GCTGAAGGACAAGGCGTGGGCAAC | GTTGCCCACGCCTTGTCCTTCAGC |
| 3160 | GGGATAGGAGACCCTCGCAATGGT | ACCATTGCGAGGGTCTCCTATCCC |
| 3161 | TTGCAGTACGTCCTTGCGCATGAA | TTCATGCGCAAGGACGTACTGCAA |
| 3162 | TTGATCACTGGATTGGGTGCGAAC | GTTCGCACCCAATCCAGTGATCAA |
| 3163 | TCTGCAGACGTTGCGAGAGATGAT | ATCATCTCTCGCAACGTCTGCAGA |
| 3164 | AGTCTAGCAGGGATCGAAGCGGAT | ATCCGCTTCGATCCCTGCTAGACT |
| 3165 | GGGGTCCCGCAACAACTAATGAAG | CTTCATTAGTTGTTGCGGGACCCC |
| 3166 | CAACCTCTTATGTGGTGTGCGCGA | TCGCGCACACCACATAAGAGGTTG |
| 3167 | CTCGCTGGGTTGCTGGAGTAGCAC | GTGCTACTCCAGCAACCCAGCGAG |
| 3168 | CGTTGTATTGTGCAACGCGAAGTT | AACTTCGCGTTGCACAATACAACG |
| 3169 | GGGCTCAAAGTGCCTGAGTCGAAA | TTTCGACTCAGGCACTTTGAGCCC |
| 3170 | CTGCTGTGCCCTCTCAGTGAGAGC | GCTCTCACTGAGAGGGCACAGCAG |
| 3171 | CGGACGTACTGTTCGGAGTCCTCA | TGAGGACTCCGAACAGTACGTCCG |
| 3172 | GTATACCACCATACCGGGACCGCA | TGCGGTCCCGGTATGGTGGTATAC |

TABLE 3

| Seq. ID No. | Decoder Sequence (5′-3′) | Probe Sequence (5′-3′) |
|---|---|---|
| 17 | TTCGCCGTCGTGTAGGCTTTTCAA | TTGAAAAGCCTACACGACGGCGAA |
| 18 | GTTCCCAGTGAAGCTGCGATCTGG | CCAGATCGCAGCTTCACTGGGAAC |
| 19 | TACTTGGCATGGAATCCCTTACGC | GCGTAAGGGATTCCATGCCAAGTA |
| 20 | ACTAGCATATTTCAGGGCACCGGC | GCCGGTGCCCTGAAATATGCTAGT |
| 21 | GAACGGTCAATGAACCCGCTGTGA | TCACAGCGGGTTCATTGACCGTTC |
| 22 | GCGGCCTTGGTTCAATATGAATCG | CGATTCATATTGAACCAAGGCCGC |
| 23 | GATCGTTAGAGGGACCTTGCCCGA | TCGGGCAAGGTCCCTCTAACGATC |
| 24 | TGGACCTAGTCCGGCAGTGACGAA | TTCGTCACTGCCGGACTAGGTCCA |
| 25 | ATAAACTACCCAGGACGGGCGGAA | TTCCGCCCGTCCTGGGTAGTTTAT |
| 26 | CATCGGTTCGCGCCAATCCAGATA | TATCTGGATTGGCGCGAACCGATG |
| 27 | GTCGGGCATAGAGCCGACCACCCT | AGGGTGGTCGGCTCTATGCCCGAC |
| 28 | CTTGGGTCATGATTCACCGTGCTA | TAGCACGGTGAATCATGACCCAAG |
| 29 | TGCCTAACGTGCTAATCAGCAGCG | CGCTGCTGATTAGCACGTTAGGCA |
| 30 | CGCATGTTGGAGCATATGCCCTGA | TCAGGGCATATGCTCCAACATGCG |
| 31 | AGCCACTGCATCAGTGCTGTTCAA | TTGAACAGCACTGATGCAGTGGCT |
| 32 | GGTTGTTTTGAGGCGTCCCACACT | AGTGTGGGACGCCTCAAAACAACC |
| 33 | TCGACCAAGAGCAAGGGCGGACCA | TGGTCCGCCCTTGCTCTTGGTCGA |
| 34 | GACATCGCTATTGCGCATGGATCA | TGATCCATGCGCAATAGCGATGTC |
| 35 | GAAATACGAAGTCTGCGGGAGTCG | CGACTCCCGCAGACTTCGTATTTC |
| 36 | TGTCATGAATGATTGATCGCGCGA | TCGCGCGATCAATCATTCATGACA |
| 37 | ATATCGGGATTCGTTCCCGGTGAA | TTCACCGGGAACGAATCCCGATAT |
| 38 | GCGAGCGTACCGAAGGGCCTAGAA | TTCTAGGCCCTTCGGTACGCTCGC |
| 39 | TTACCGGCAGCGGACTTCCGAATT | AATTCGGAAGTCCGCTGCCGGTAA |
| 40 | GTAATCGAGAGCTGCGCGCCGTCT | AGACGGCGCGCAGCTCTCGATTAC |
| 41 | CCTGTTAGCGTAGGCGAGTCGATC | GATCGACTCGCCTACGCTAACAGG |
| 42 | TAGCGGACCGGCAGAATGAGTTCC | GGAACTCATTCTGCCGGTCCGCTA |
| 43 | GGTACATGCACTACGCGCACTCGG | CCGAGTGCGCGTAGTGCATGTACC |
| 44 | AATTCATCTCGGACTCCCGCGGTA | TACCGCGGGAGTCCGAGATGAATT |
| 45 | GCCAAATCTGGATTGGCAGGAATG | CATTCCTGCCAATCCAGATTTGGC |
| 46 | TGCATTTTCGGTTGAGGCACATCC | GGATGTGCCTCAACCGAAAATGCA |
| 47 | CCGCTCAATTCACCATGCTTCGCT | AGCGAAGCATGGTGAATTGAGCGG |
| 48 | CTCGGAAAGGTGCAACTTTGGTGT | ACACCAAAGTTGCACCTTTCCGAG |
| 49 | AATTCGACCAGCAGAACGTCCCAT | ATGGGACGTTCTGCTGGTCGAATT |
| 50 | GCCAGAGTCTCAACCTCACGGGAT | ATCCCGTGAGGTTGAGACTCTGGC |
| 51 | CCAACAACTGGAACGGGAACCCGC | GCGGGTTCCCGTTCCAGTTGTTGG |
| 52 | GAGAACTGATCGCTGAGGGGCATG | CATGCCCCTCAGCGATCAGTTCTC |
| 53 | GGCACACTAGACTTGTGGCACCGA | TCGGTGCCACAAGTCTAGTGTGCC |
| 54 | TCACATCCAAATATGGTCCGCGAA | TTCGCGGACCATATTTGGATGTGA |
| 55 | GTCTGCCGGTGTGACCGCTTCATT | AATGAAGCGGTCACACCGGCAGAC |
| 56 | CATCGCAGAGCATAAACACCCTCA | TGAGGGTGTTTATGCTCTGCGATG |
| 57 | GTTGGTATCTATGGCAGAGGCGGA | TCCGCCTCTGCCATAGATACCAAC |
| 58 | ACGAGGTGCCGCTGAGGTTCCATT | AATGGAACCTCAGCGGCACCTCGT |
| 59 | GGAATGAGTGGACCCAGGCACATT | AATGTGCCTGGGTCCACTCATTCC |
| 60 | TGTCAATATGCGTCCGTGTCGTCT | AGACGACACGGACGCATATTGACA |
| 61 | TGATGAGCCTCAGGGTACGAGGCA | TGCCTCGTACCCTGAGGCTCATCA |
| 62 | CACCGCGGTGTTCCTACAGAATGA | TCATTCTGTAGGAACACCGCGGTG |
| 63 | TTGTTGCCAATGGTGTCCGCTCGG | CCGAGCGGACACCATTGGCAACAA |
| 64 | TTAACCTGCGTCTGCCCCTTTCCT | AGGAAAGGGGCAGACGCAGGTTAA |
| 65 | AGGCGCGTTCCTGCCTTAGTGACG | CGTCACTAAGGCAGGAACGCGCCT |
| 66 | TAGGGCGATGGCACGAAGCTTCAA | TTGAAGCTTCGTGCCATCGCCCTA |
| 67 | TGCATAGAGCCAAAGTCGGCGATG | CATCGCCGACTTTGGCTCTATGCA |
| 68 | TTGAGAGGCAGGTGGCCACACGGA | TCCGTGTGGCCACCTGCCTCTCAA |
| 69 | TCCGCATTGTGAGAAAAAACGAGC | GCTCGTTTTTTCTCACAATGCGGA |
| 70 | GGCGGTTTCCGTAGCTATAGGTGC | GCACCTATAGCTACGGAAACCGCC |
| 71 | GGTGAAAATTTCGTAGCCACGGGC | GCCCGTGGCTACGAAATTTTCACC |
| 72 | CCGACGGAGGATGAAGACAATCAC | GTGATTGTCTTCATCCTCCGTCGG |
| 73 | CCAGTTTGGCCCAATTCGCCAAAA | TTTTGGCGAATTGGGCCAAACTGG |
| 74 | GGATCTATTAGGCCGTGCGCACAG | CTGTGCGCACGGCCTAATAGATCC |
| 75 | CGGATGTCACCGTTTGGACTTTCA | TGAAAGTCCAAACGGTGACATCCG |
| 76 | ATCGCAAATCCTGCTCGTCCCTAA | TTAGGGACGAGCAGGATTTGCGAT |
| 77 | CAGGGCATGCAATAATCGAGGTTC | GAACCTCGATTATTGCATGCCCTG |
| 78 | CATGCGTTGATATATGGGCCCAAG | CTTGGGCCCATATATCAACGCATG |
| 79 | CAGCTGCAGCTTGTGACCAACCAC | GTGGTTGGTCACAAGCTGCAGCTG |
| 80 | TTGTATGTCTGCCGACCGGCGACC | GGTCGCCGGTCGGCAGACATACAA |
| 81 | GATGGCGCCCGTTGATAGGTATGG | CCATACCTATCAACGGGCGCCATC |
| 82 | ATGAGAATCGCCGGCAATCTGCTA | TAGCAGATTGCCGGCGATTCTCAT |
| 83 | ATTTGCACTGACCGCAGGCTCGTG | CACGAGCCTGCGGTCAGTGCAAAT |
| 84 | CAGGGAGAACGGTTAAGTTCCCGT | ACGGGAACTTAACCGTTCTCCCTG |
| 85 | AGGCCGGCGATCGAGGAGTTTGGT | ACCAAACTCCTCGATCGCCGGCCT |
| 86 | ACACGGTGGTCTCTGATAGCGACC | GGTCGCTATCAGAGACCACCGTGT |
| 87 | GTGCAACGCCGAGGACTTCCATCA | TGATGGAAGTCCTCGGCGTTGCAC |
| 88 | TCGGTGCCTGATAGCCATTCCGAT | ATCGGAATGGCTATCAGGCACCGA |
| 89 | TGAAATACCACACAGCCAATTGGC | GCCAATTGGCTGTGTGGTATTTCA |
| 90 | GCATCGTGTACATGACTGCCGCGA | TCGCGGCAGTCATGTACACGATGC |
| 91 | CAGTGTTCTAACGGCGCGCGTGAA | TTCACGCGCGCCGTTAGAACACTG |
| 92 | CGCTTGCAACGTTGCACCTACTCT | AGAGTAGGTGCAACGTTGCAAGCG |
| 93 | CGAAAAACTAGTGGGCTCGCCGCG | CGCGGCGAGCCCACTAGTTTTTCG |
| 94 | CTTTCAGGGGAACTGCCGGAGTCG | CGACTCCGGCAGTTCCCCTGAAAG |
| 95 | TTGTGGCCTTCTTGTAAAGGCACG | CGTGCCTTTACAAGAAGGCCACAA |
| 96 | TCCACGAACGGCGACCCGTTGTCT | AGACAACGGGTCGCCGTTCGTGGA |
| 97 | CGACCTTGCACGAAACCTAACGAG | CTCGTTAGGTTTCGTGCAAGGTCG |
| 98 | GTGCAGCTTCACGAGCCAGCCTGA | TCAGGCTGGCTCGTGAAGCTGCAC |
| 99 | CGCTTTCGTGCGAATAGACGATGA | TCATCGTCTATTCGCACGAAAGCG |
| 100 | TGCGCTTACAGGCTCCTAGTGGTC | GACCACTAGGAGCCTGTAAGCGCA |
| 101 | CACGCGCTTAGTCGCGATCGCATA | TATGCGATCGCGACTAAGCGCGTG |
| 102 | CGGAGGGAGGGAGCTAGCCTTCGA | TCGAAGGCTAGCTCCCTCCCTCCG |
| 103 | GCATCCGGCCTGTTGATGACGCCT | AGGCGTCATCAACAGGCCGGATGC |
| 104 | AGGCCAATCGATCTTATTGCCGAG | CTCGGCAATAAGATCGATTGGCCT |
| 105 | CCTTCCAATGATTGCATACGCCCA | TGGGCGTATGCAATCATTGGAAGG |
| 106 | AACACTTGATCAGGCGGGTCGTCT | AGACGACCCGCCTGATCAAGTGTT |
| 107 | TGGAATCAAGGCCGTAAAGGACAG | CTGTCCTTTACGGCCTTGATTCCA |
| 108 | GCTCCCGTAACCTGTCCACCAGTG | CACTGGTGGACAGGTTACGGGAGC |
| 109 | AGTGGTGAATGGCCGCTACCCTGA | TCAGGGTAGCGGCCATTCACCACT |
| 110 | TGTTGAAGCGAGCTAAAACGGCCA | TGGCCGTTTTAGCTCGCTTCAACA |
| 111 | CAGCGCTCCAGAATTGACAGCAAT | ATTGCTGTCAATTCTGGAGCGCTG |
| 2 | TTCGAAGCGCACGTCCCTTTTCAA | TTGAAAAGGGACGTGCGCTTCGAA |
| 3 | AACGCGTGGGGAATGGGACATCAA | TTGATGTCCCATTCCCCACGCGTT |
| 114 | CACGAGATACCGGCGTAAGGGTGG | CCACCCTTACGCCGGTATCTCGTG |
| 115 | CTACGGCAAACGTGTGGAATGGGT | ACCCATTCCACACGTTTGCCGTAG |
| 116 | GTAGGGCGATGACGGGCGAACTAC | GTAGTTCGCCCGTCATCGCCCTAC |
| 117 | AATCGACCTCCGCACACATTCGCA | TGCGAATGTGTGCGGAGGTCGATT |
| 118 | GAGTCAGCATGGCGGCGGAGATTC | GAATCTCCGCCGCCATGCTGACTC |
| 119 | AGATAAAGACGCTGGCAACACGGG | CCCGTGTTGCCAGCGTCTTTATCT |
| 120 | GGTACCTCAACGCGAACCACTTGT | ACAAGTGGTTCGCGTTGAGGTACC |
| 121 | AAGCGATGGCTACCCAAGAGCGAT | ATCGCTCTTGGGTAGCCATCGCTT |
| 122 | AGAGCTTATGCAGAACCAGGCGCC | GGCGCCTGGTTCTGCATAAGCTCT |
| 123 | ATCGGTCTCACGCAGGGTTGGATA | TATCCAACCCTGCGTGAGACCGAT |
| 124 | TAGGTTGCCCGCCAGAAGAAACAT | ATGTTTCTTCTGGCGGGCAACCTA |
| 125 | CGGTGCTGTTGCAAAAGCCTGTAG | CTACAGGCTTTTGCAACAGCACCG |
| 126 | TGATGAAAGTTTGCGGCAGGACAC | GTGTCCTGCCGCAAACTTTCATCA |
| 127 | GTTGAGTGCAGGATGCAGCGATAG | CTATCGCTGCATCCTGCACTCAAC |
| 128 | AACATTGCGCGGTCCACCAGGGTT | AACCCTGGTGGACCGCGCAATGTT |
| 129 | GGGCAGTTAGAGAGGGCCAGAAGT | ACTTCTGGCCCTCTCTAACTGCCC |
| 130 | TCGAGCTGGTCCCCGTGAACGTGT | ACACGTTCACGGGGACCAGCTCGA |
| 131 | GTCTTGGGGGCCGCTTAGTGAAAA | TTTTCACTAAGCGGCCCCCAAGAC |
| 132 | ACTGTTGGCTTGCTCTCATGTCCA | TGGACATGAGAGCAAGCCAACAGT |
| 133 | AGGACCATTCGGAAGGCGAAGATA | TATCTTCGCCTTCCGAATGGTCCT |
| 134 | CTTGGGAGGCATCCGCTATAAGGA | TCCTTATAGCGGATGCCTCCCAAG |
| 135 | AATAAACGGAACGCACCGCTACAG | CTGTAGCGGTGCGTTCCGTTTATT |
| 136 | TTGTACGTGCGGTCCCCATAAGCA | TGCTTATGGGGACCGCACGTACAA |
| 137 | CGCACCAAACTGAGTTTCCCAGAC | GTCTGGGAAACTCAGTTTGGTGCG |
| 138 | ACCTGATCGTTCCCCTATTGGGAA | TTCCCAATAGGGGAACGATCAGGT |
| 139 | GGAACAGAGGCGAGGGGACTGAGC | GCTCAGTCCCCTCGCCTCTGTTCC |
| 140 | CCCTGCCTTGGCGTGTCGGCTTAT | ATAAGCCGACACGCCAAGGCAGGG |
| 141 | ACTCTGACACGCCAACTCCGGAAG | CTTCCGGAGTTGGCGTGTCAGAGT |
| 142 | CTGACGGTTTTCATTCGGCGTGCC | GGCACGCCGAATGAAAACCGTCAG |
| 143 | TGCGGTGGTTCATTGGAGCTGGCC | GGCCAGCTCCAATGAACCACCGCA |
| 144 | GCATGGCCAACTAGTGACTCGCAA | TTGCGAGTCACTAGTTGGCCATGC |
| 145 | AGGCCGTAAAGCGAATCTCACCTG | CAGGTGAGATTCGCTTTACGGCCT |
| 146 | CGAATATTATGCCGAGAATCCGCG | CGCGGATTCTCGGCATAATATTCG |
| 147 | ACAGACGAGCTCCCAACCACATGA | TCATGTGGTTGGGAGCTCGTCTGT |
| 148 | GGACGGTTTGTGCTGGATTGTCTG | CAGACAATCCAGCACAAACCGTCC |
| 149 | AAAGGCTATTGAGTTGGTTGGGCG | CGCCCAACCAACTCAATAGCCTTT |
| 150 | GATGGCCTATTCGGAGATCGGGCC | GGCCCGATCTCCGAATAGGCCATC |
| 151 | GATCCAGTAGGCAGCTTCATCCCA | TGGGATGAAGCTGCCTACTGGATC |
| 152 | AATAACTCGCGCGGGTATGCTTCT | AGAAGCATACCCGCGCGAGTTATT |
| 153 | GGAGGAGGTTTGTCTCGGAAAGCA | TGCTTTCCGAGACAAACCTCCTCC |
| 154 | CTTTGGTATGGCACATGCTGCCCG | CGGGCAGCATGTGCCATACCAAAG |
| 155 | AGAAAGGCTCGAGCAACGGGAACT | AGTTCCCGTTGCTCGAGCCTTTCT |
| 156 | AATCTACCGCACTGGTCCGCAAGT | ACTTGCGGACCAGTGCGGTAGATT |
| 157 | CGTGGCGGCCACAGTTTTTGGAGG | CCTCCAAAAACTGTGGCCGCCACG |
| 158 | TTGCAGTTCAATCCATACGCACGT | ACGTGCGTATGGATTGAACTGCAA |
| 159 | GGCCCAAAGCCCCAGACCATTTTA | TAAAATGGTCTGGGGCTTTGGGCC |
| 160 | CGCCTGTCTTTGTCTCCGGACAAT | ATTGTCCGGAGACAAAGACAGGCG |
| 161 | TGAGGCAACAGGGGCCAAAAACTA | TAGTTTTTGGCCCCTGTTGCCTCA |
| 162 | AGCGGAAGTAGTCCTCGGCTCGTC | GACGAGCCGAGGACTACTTCCGCT |
| 163 | GGCCCCAAGGCTTAGAGATAGTGG | CCACTATCTCTAAGCCTTGGGGCC |
| 164 | GCACGTGAAGTTTAACCGCGATTC | GAATCGCGGTTAAACTTCACGTGC |
| 165 | AGCGGCAGAAACGTTCCTTGACGG | CCGTCAAGGAACGTTTCTGCCGCT |
| 166 | TCGTCGAGCAGACGAGATTGCACG | CGTGCAATCTCGTCTGCTCGACGA |
| 167 | TCTTTGCCGCGTAACTGACTGCTT | AAGCAGTCAGTTACGCGGCAAAGA |
| 168 | TTTATGTGCCAAGGGGTTAACCGA | TCGGTTAACCCCTTGGCACATAAA |
| 169 | TGTTACTGTGGTTCACGGCAGTCC | GGACTGCCGTGAACCACAGTAACA |
| 170 | CGCGCCTCGCTAGACCTTTTATTG | CAATAAAAGGTCTAGCGAGGCGCG |
| 171 | ACAAATGCGTGAGAGCTCCCAACT | AGTTGGGAGCTCTCACGCATTTGT |
| 172 | CGCGCAGATTATAGACCCGAATGT | ACATTCGGGTCTATAATCTGCGCG |
| 173 | CAAATAACGCCGCTGAATCGGCGT | ACGCCGATTCAGCGGCGTTATTTG |
| 174 | CCTTCGTGCATCGGTGATGATGTT | AACATCATCACCGATGCACGAAGG |
| 175 | TGAACACGAGCAACACTCCAACGC | GCGTTGGAGTGTTGCTCGTGTTCA |
| 176 | CAGCAGATCCTTCGTAGCGGTCGT | ACGACCGCTACGAAGGATCTGCTG |
| 177 | GGAACCTGGTGAGTTGTGCCTCAT | ATGAGGCACAACTCACCAGGTTCC |
| 178 | TCATAAGCGACAATCGCGGGCTTA | TAAGCCCGCGATTGTCGCTTATGA |
| 179 | CCCAACGTCACTGAAGCTCACAGT | ACTGTGAGCTTCAGTGACGTTGGG |
| 180 | TGTCAGAGCCCGCGACTCAGACGG | CCGTCTGAGTCGCGGGCTCTGACA |
| 181 | TACACGAAGCCTCTCCGTGGTCCA | TGGACCACGGAGAGGCTTCGTGTA |
| 182 | CTCAGAAGTCCTCGGCGAACTGGG | CCCAGTTCGCCGAGGACTTCTGAG |
| 183 | ATCCTTTTATCTACTCCGCGGCGA | TCGCCGCGGAGTAGATAAAAGGAT |
| 184 | AGGCGTGCAGCAACAGGATAAACC | GGTTTATCCTGTTGCTGCACGCCT |
| 185 | ACTCTCGAGGGAGTCTCTGGCACA | TGTGCCAGAGACTCCCTCGAGAGT |
| 186 | TTGCCAGGTCCATCGAGACCTGTT | AACAGGTCTCGATGGACCTGGCAA |
| 187 | TCCACTATAACTGCGGGTCCGTGT | ACACGGACCCGCAGTTATAGTGGA |
| 188 | GCCCAGTCGGCTCTAACAAGTTCG | CGAACTTGTTAGAGCCGACTGGGC |
| 189 | CGGAACGGATAATCGGCGTCAGGT | ACCTGACGCCGATTATCCGTTCCG |
| 190 | TAAAATAAGCGCCTGGCGGGAGGA | TCCTCCCGCCAGGCGCTTATTTTA |
| 191 | GCGCACTCGTGAAACCTTTCTCGC | GCGAGAAAGGTTTCACGAGTGCGC |
| 192 | AGTTTGCCAGGTACTGGCAAGTGC | GCACTTGCCAGTACCTGGCAAACT |
| 193 | ACAACGAGGGATGTCCAGCGGCAT | ATGCCGCTGGACATCCCTCGTTGT |
| 194 | TTCGCAGCACCCGCTAGGTACAGT | ACTGTACCTAGCGGGTGCTGCGAA |
| 195 | TAACCCGATTTTTGCGACTCTGCC | GGCAGAGTCGCAAAAATCGGGTTA |
| 196 | CGTCGCATTGCAAGCGTAGGCTTG | CAAGCCTACGCTTGCAATGCGACG |
| 197 | GAGCTGACGTCACCATCAGAGGAA | TTCCTCTGATGGTGACGTCAGCTC |
| 198 | GGAGGCTGGGGGTCGCGCTTAAGT | ACTTAAGCGCGACCCCCAGCCTCC |
| 199 | TTGTGGGAACCGCACTAGCTGGCT | AGCCAGCTAGTGCGGTTCCCACAA |
| 200 | CCCTCGCACTGTGTTCACCCTCTT | AAGAGGGTGAACACAGTGCGAGGG |
| 201 | TCATTGACTCGAATCCGCACAACG | CGTTGTGCGGATTCGAGTCAATGA |
| 202 | ACAGGGGTTGGCCTTCGTACGTAC | GTACGTACGAAGGCCAACCCCTGT |
| 203 | AGGCCGTGCAACATCACACAGGAT | ATCCTGTGTGATGTTGCACGGCCT |
| 204 | GGGCCGTGGTCACGTAATATTGGC | GCCAATATTACGTGACCACGGCCC |
| 205 | GCGCGGACATGAAACGACAAGGCC | GGCCTTGTCGTTTCATGTCCGCGC |
| 206 | CTTATTGGGTGCCGGTGTCGGATT | AATCCGACACCGGCACCCAATAAG |
| 207 | GGGGCGGTTACCAAAAAATCCGAT | ATCGGATTTTTTGGTAACCGCCCC |
| 4 | CCGTCGCATACCGGCTACGATCAA | TTGATCGTAGCCGGTATGCGACGG |
| 5 | ATGGCCGTGCTGGGGACAAGTCAA | TTGACTTGTCCCCAGCACGGCCAT |
| 210 | ACGAAAAAAGTGTGCGGATCCCCT | AGGGGATCCGCACACTTTTTTCGT |
| 211 | CCAAGTACACCGCACGCATGTTTA | TAAACATGCGTGCGGTGTACTTGG |
| 212 | ATCGTGCGTGGAGTGTCGCATCTA | TAGATGCGACACTCCACGCACGAT |
| 213 | TCCAGATACCGCCCCGAACTTTGA | TCAAAGTTCGGGGCGGTATCTGGA |
| 214 | TCTGCTGGCAGCACGTGAAGTGGC | GCCACTTCACGTGCTGCCAGCAGA |
| 215 | TTGAAATTGCTCTGCCGTCAGTCA | TGACTGACGGCAGAGCAATTTCAA |
| 216 | AGTCAGGCGAGATGTTCAGGCAGC | GCTGCCTGAACATCTCGCCTGACT |
| 217 | ACAAGCCGACGTTAAGCCCGCCCA | TGGGCGGGCTTAACGTCGGCTTGT |
| 218 | CCCTAATGAGGCCAGTAACCTGCA | TGCAGGTTACTGGCCTCATTAGGG |
| 219 | GTGAGACACACATCCCCTCCAATG | CATTGGAGGGGATGTGTGTCTCAC |
| 220 | CGACGGATGCAGAGTTCAGTGGTC | GACCACTGAACTCTGCATCCGTCG |
| 221 | CCCGCATGCCTGGCGGTATTACAA | TTGTAATACCGCCAGGCATGCGGG |
| 222 | TTAGCAAAGCGGCGCCGTTAGCAA | TTGCTAACGGCGCCGCTTTGCTAA |
| 223 | CCCGACACGGGTCAGCGTAATAAT | ATTATTACGCTGACCCGTGTCGGG |
| 224 | GCGACGGCCCTGAGGTATGTCGTC | GACGACATACCTCAGGGCCGTCGC |
| 225 | CAAAAGTGTGTTCCCTTGCGCTTG | CAAGCGCAAGGGAACACACTTTTG |
| 226 | TCTCGAAGCACAGCCCGGTTATTG | CAATAACCGGGCTGTGCTTCGAGA |
| 227 | ATGCTAACCGTTGGCCATGGAACT | AGTTCCATGGCCAACGGTTAGCAT |
| 228 | CTTGCGGAGTGTTAGCCCAGCGGT | ACCGCTGGGCTAACACTCCGCAAG |
| 229 | TGCTCCCTAGGCGCTCGGAGGAGT | ACTCCTCCGAGCGCCTAGGGAGCA |
| 230 | CCAATGCCTTTGAGTAAGCGATGG | CCATCGCTTACTCAAAGGCATTGG |
| 231 | AGCAGATAACGTCCCAATGACGCC | GGCGTCATTGGGACGTTATCTGCT |
| 232 | TTGACCATTACGTGTTGCGCCCAT | ATGGGCGCAACACGTAATGGTCAA |
| 233 | TCGCGTATTTGCGGAATTCGTCTG | CAGACGAATTCCGCAAATACGCGA |
| 234 | CTGCGTGTCAACAATGTCCCGCAG | CTGCGGGACATTGTTGACACGCAG |
| 235 | TCTGGTGCCACGCAAGGTCCACAG | CTGTGGACCTTGCGTGGCACCAGA |
| 236 | CTCCGGGAGGTCACTTAATTGCGG | CCGCAATTAAGTGACCTCCCGGAG |
| 237 | TTTTCGTGATTGCCCGGAGGAGGC | GCCTCCTCCGGGCAATCACGAAAA |
| 238 | TCGGGATGTAGCTGGGGCTACCGG | CCGGTAGCCCCAGCTACATCCCGA |
| 239 | CGAGCCAACGCAAACACGTCCTTG | CAAGGACGTGTTTGCGTTGGCTCG |
| 240 | GCAAAGCCTTTGTGGGGCGGTAGT | ACTACCGCCCCACAAAGGCTTTGC |
| 241 | ATTCGACCGGAAATGAGGTCTTCG | CGAAGACCTCATTTCCGGTCGAAT |
| 242 | TTCGCTTGCTGAGTTGGTCTGTTC | GAACAGAGCAACTCAGCAAGCGAA |
| 243 | CGCGTGAAGACCCCATTCCCGAGT | ACTCGGGAATGGGGTCTTCACGCG |
| 244 | AACCGTATTCGCGGTCACTTGTGG | CCACAAGTGACCGCGAATACGGTT |
| 245 | GGGGCCAACCGTTTCGAGGCGTAT | ATACGCCTCGAAACGGTTGGCCCC |
| 246 | TTCGGCTGGCAGTCCAAACGGCTT | AAGCCGTTTGGACTGCCAGCCGAA |
| 247 | GGGTGTGGTTAGAATGCACGGTTC | GAACCGTGCATTCTAACCACACCC |
| 248 | GCGAGGACCGAACTAGACAAACGG | CCGTTTGTCTAGTTCGGTCCTCGC |
| 249 | ACGCACGCGTGACCGAAGTTGCTG | CAGCAACTTCGGTCACGCGTGCGT |
| 250 | TAAAAGGTCGCTTTGAAAGGGGGA | TCCCCCTTTCAAAGCGACCTTTTA |
| 251 | TGCGATCGCTAACTGCTGGGACAA | TTGTCCCAGCAGTTAGCGATCGCA |
| 252 | GGAGGTATAAGCGGAGCGGCCTCA | TGAGGCCGCTCCGCTTATACCTCC |
| 253 | ATGCTGACATGTCGTGCACCTCGT | ACGAGGTGCACGACATGTCAGCAT |
| 254 | TGTGGTTAAAGCGTCCGTTCAACG | CGTTGAACGGACGCTTTAACCACA |
| 255 | CGTTCACACCGGCGTAAGCTGCGT | ACGCAGCTTACGCCGGTGTGAACG |
| 256 | CCTATCCCGGCGAGAACTTCTGTG | CACAGAAGTTCTCGCCGGGATAGG |
| 257 | GTCTGCACTCACGCAGCGGAGGGA | TCCCTCCGCTGCGTGAGTGCAGAC |
| 258 | GCACGAGTTGGTGCTCGGCAGATT | AATCTGCCGAGCACCAACTCGTGC |
| 259 | AACGTCGCACGACACACGTTCGTC | GACGAACGTGTGTCGTGCGACGTT |
| 260 | ATGCGCGCTTATCCTAGCATGGTC | GACCATGCTAGGATAAGCGCGCAT |
| 261 | TCACGTTTTCGTCTCGACATGAGG | CCTCATGTCGAGACGAAAACGTGA |
| 262 | TGTGCCTCATCCTTAGGATACGGC | GCCGTATCCTAAGGATGAGGCACA |
| 263 | AGGTGGTGTGGGTCAACCGCTTTA | TAAAGCGGTTGACCCACACCACCT |
| 264 | CTGGATCGAAGGGACTGCAAGCTC | GAGCTTGCAGTCCCTTCGATCCAG |
| 265 | TAGATCAACTCGCGTACGCATGGA | TCCATGCGTACGCGAGTTGATCTA |
| 266 | GATCCTGCGGAGAAGAGAGTGCAG | CTGCACTCTCTTCTCCGCAGGATC |
| 267 | TACGTGTGGAGATGCCCCGAACCG | CGGTTCGGGGCATCTCCACACGTA |
| 268 | GCGCTATGTCAATCGTGGGCGTAG | CTACGCCCACGATTGACATAGCGC |
| 269 | AGCGAGGTTTCTAGCGTCGACACC | GGTGTCGACGCTAGAAACCTCGCT |
| 270 | ACCCAGGTTTTGCCGTTGTGGAAT | ATTCCACAACGGCAAAACCTGGGT |
| 271 | CCCTGTTAACGGCTGCGTAGTCTC | GAGACTACGCAGCCGTTAACAGGG |
| 272 | AGGCCGATTTCACCCGCCAATTGC | GCAATTGGCGGGTGAAATCGGCCT |
| 273 | GAGCCCTCACTCCTTGCCCTTTGA | TCAAAGGGCAAGGAGTGAGGGCTC |
| 274 | GGGTGGACATCCGCCTCGCAGTCA | TGACTGCGAGGCGGATGTCCACCC |
| 275 | GATGGCTGAGAACCGTGCTACGAT | ATCGTAGCACGGTTCTCAGCCATC |
| 276 | TCGACGTTAGGAGTGCTGCCAGAA | TTCTGGCAGCACTCCTAACGTCGA |
| 277 | CGAATGGGTCTGGACCTTGCATAG | CTATGCAAGGTCCAGACCCATTCG |
| 278 | GTGCACCAGACATTCGAACTCGGA | TCCGAGTTCGAATGTCTGGTGCAC |
| 279 | AGAGGCCCCGTATATCCCATCCAT | ATGGATGGGATATACGGGGCCTCT |
| 280 | AACGCCTGTTCAGAGCATCAGCGG | CCGCTGATGCTCTGAACAGGCGTT |
| 281 | AAGGCTCAACACGCCTATGTGCGC | GCGCACATAGGCGTGTTGAGCCTT |
| 282 | AGTCCGTGTTGCCAGATTGGCTCG | CGAGCCAATCTGGCAACACGGACT |
| 283 | ATGTCCCATGTAAAGACGCGTGTG | CACACGCGTCTTTACATGGGACAT |
| 284 | ATGGAGTCTGCTCACGCCCAAAGG | CCTTTGGGCGTGAGCAGACTCCAT |
| 285 | CGGCCTCCAACAAGGAGCACTAAC | GTTAGTGCTCCTTGTTGGAGGCCG |
| 286 | CAGAGCCGTGGCAACATTGCGAGC | GCTCGCAATGTTGCCACGGCTCTG |
| 287 | TCATTTGAATGAGGTGCGCACCGG | CCGGTGCGCACCTCATTCAAATGA |
| 288 | GACGTACCGGAAGCGCCGTATAAA | TTTATACGGCGCTTCCGGTACGTC |
| 289 | ATGCGAGCAATGGGATCCGGATTC | GAATCCGGATCCCATTGCTCGCAT |
| 290 | AGAGTGAGGCCTCCCTGACCAGTG | CACTGGTCAGGGAGGCCTCACTCT |
| 291 | CGCACCGTAAGTAGATTTGCCCGC | GCGGGCAAATCTACTTACGGTGCG |
| 292 | TGAACCTTTGAGCACGTCGTGCGC | GCGCACGACGTGCTCAAAGGTTCA |
| 293 | TCCGCCTTTTTGGTTACCTCGAAG | CTTCGAGGTAACCAAAAAGGCGGA |
| 294 | GAACGCCAACGGCACTAACACATC | GATGTGTTAGTGCCGTTGGCGTTC |
| 295 | CCGACAGCAGCCAAGACGTCCCAG | CTGGGACGTCTTGGCTGCTGTCGG |
| 296 | CATAAAAAAACCTGGGGCTCTGCG | CGCAGAGCCCCAGGTTTTTTTATG |
| 297 | TGCCAACTGTGCAGACCGGACTTA | TAAGTCCGGTCTGCACAGTTGGCA |
| 298 | GGCGAAAGAGCGAAACCGGCTCGT | ACGAGCCGGTTTCGCTCTTTCGCC |
| 299 | GGGATGCGTATTTTAGCGAACACG | CGTGTTCGCTAAAATACGCATCCC |
| 300 | TGGGATTCAGCGACCAGTACGCGA | TCGCGTACTGGTCGCTGAATCCCA |
| 301 | CCCGATATTCGCCCGGCCTATTCG | CGAATAGGCCGGGCGAATATCGGG |
| 302 | CGAGAAGATGCCTCACGCAACCAA | TTGGTTGCGTGAGGCATCTTCTCG |
| 303 | AACCTTGACCCGTGGATGACGCTA | TAGCGTCATCCACGGGTCAAGGTT |
| 6 | TTGCAACGGGCTGGTCAACGTCAA | TTGACGTTGACCAGCCCGTTGCAA |
| 7 | CGCATAGGTTGCCGATTTCGTCAA | TTGACGAAATCGGCAACCTATGCG |
| 306 | GCTTCCGGATGAACGGGATGGTTG | CAACCATCCCGTTCATCCGGAAGC |
| 307 | CCCTCCATGTTCTTCGAACGGTTT | AAACCGTTCGAAGAACATGGAGGG |
| 308 | TTGATGGGCGGCAATGCTCTTGCT | AGCAAGAGCATTGCCGCCCATCAA |
| 309 | ATTGTGAGATGCGCCAAATTCCCC | GGGGAATTTGGCGCATCTCACAAT |
| 310 | TCAGCACAGCCAGACGGTCAACTT | AAGTTGACCGTCTGGCTGTGCTGA |
| 311 | ACTCCACTCCTCGGTGGCAAACTA | TAGTTTGCCACCGAGGAGTGGAGT |
| 312 | TCTGGGCATGCCTGGACGGAGACG | CGTCTCCGTCCAGGCATGCCCAGA |
| 313 | TCTCAACTCCGGTACGACGAAACA | TGTTTCGTCGTACCGGAGTTGAGA |
| 314 | TTGCGTGGTCAAAGGCGCAACGTG | CACGTTGCGCCTTTGACCACGCAA |
| 315 | AGACAGCGATCCGCGGCTCATGAT | ATCATGAGCCGCGGATCGCTGTCT |
| 316 | CGCGTCTCTAACTGAGAGCAGCCA | TGGCTGCTCTCAGTTAGAGACGCG |
| 317 | AGGCGCACATGTACGGACATTCAG | CTGAATGTCCGTACATGTGCGCCT |
| 318 | GATGAGTGGCACGTCGGTGTGTAA | TTACACACCGACGTGCCACTCATC |
| 319 | TGATCCATATTGTCGGACGTTGCG | CGCAACGTCCGACAATATGGATCA |
| 320 | ACCTGCCGGGAGTTCATAGGCTAG | CTAGCCTATGAACTCCCGGCAGGT |
| 321 | AGCATTGGCGTTTTTCCGCAACGA | TCGTTGCGGAAAAACGCCAATGCT |
| 322 | GGTAATATTCAGCGCGACCGCTCA | TGAGCGGTCGCGCTGAATATTACC |
| 323 | ATAGCGTACGACGAGGTGACGCGC | GCGCGTCACCTCGTCGTACGCTAT |
| 324 | TAGGTCACGATGCGTTTGACGCTA | TAGCGTCAAACGCATCGTGACCTA |
| 325 | ACTGCCCGTACCTCTGGTTCTGGC | GCCAGAACCAGAGGTACGGGCAGT |
| 326 | CCTTTGGCCTGAAGTTGTCGTAGC | GCTACGACAACTTCAGGCCAAAGG |
| 327 | GTGCCCCACGAGCGTATCGTTGTA | TACAACGATACGCTCGTGGGGCAC |
| 328 | AGGCGCTACGTGGGCCTGGAGCAA | TTGCTCCAGGCCCACGTAGCGCCT |
| 329 | GGGTGCTACCATTGCATTAGTCCG | CGGACTAATGCAATGGTAGCACCC |
| 330 | ACCACGCGCGTACGTGTAACCGAG | CTCGGTTACACGTACGCGCGTGGT |
| 331 | CCATGATGCATTGGGTGCATTTAG | CTAAATGCACCCAATGCATCATGG |
| 332 | GGTCCGGCCCTACGAAACGTTCGA | TCGAACGTTTCGTAGGGCCGGACC |
| 333 | CCGTGTGGCTGGAGATTCGTGTGA | TCACACGAATCTCCAGCCACACGG |
| 334 | GTTAGGGCGACGCATATTGGCACA | TGTGCCAATATGCGTCGCCCTAAC |
| 335 | GGGTCAGTCAGGTGCGTTAGGATC | GATCCTAACGCACCTGACTGACCC |
| 336 | GCCGTGAAGTCGAATGCAGATCGA | TCGATCTGCATTCGACTTCACGGC |
| 337 | GCCACCACCCAGTGCATTCAGGTA | TACCTGAATGCACTGGGTGGTGGC |
| 338 | GAGCTTAGTTTGCGGTCATCGGGC | GCCCGATGACCGCAAACTAAGCTC |
| 339 | TGTTTGCCGCCATTAGGGAGTAAC | GTTACTCCCTAATGGCGGCAAACA |
| 340 | GCTCCGCTGGATGTGCCGGTTTAG | CTAAACCGGCACATCCAGCGGAGC |
| 341 | CGGTAGCATGCGAGATCCCTGTTA | TAACAGGGATCTCGCATGCTACCG |
| 342 | CTACGCTCTACCAGTTGCCTGCGA | TCGCAGGCAACTGGTAGAGCGTAG |
| 343 | GTGCCTCCTGCTGTATTTGCCAAG | CTTGGCAAATACAGCAGGAGGCAC |
| 344 | TTGCGACTCGACTTGGACGAGTAG | CTACTCGTCCAAGTCGAGTCGCAA |
| 345 | TCTGGGAGCTGTTTACTCCAGCCA | TGGCTGGAGTAAACAGCTCCCAGA |
| 346 | TGGACGCGGAACTCCCTTTAGCAT | ATGGTAAAGGGAGTTCCGCGTGCA |
| 347 | TGGCAGCAAATGAATCGAAAGCAC | GTGCTTTCGATTCATTTGCTGCCA |
| 348 | AACTGGTGACGCGGTACAGCGAAG | CTTCGCTGTACCGCGTCACCAGTT |
| 349 | AGACGATTACGCTGGACGCCGTCG | CGACGGCGTCCAGCGTAATCGTCT |
| 350 | ATGCCCTCCTTCATGGAAAGGGTT | AACCCTTTCCATGAAGGAGGGCAT |
| 351 | ATTCTCGGAGCGTATGCGCCAGAA | TTCTGGCGCATACGCTCCGAGAAT |
| 352 | ATAGCGGAGTTTGGGTACGCGAAC | GTTCGCGTACCCAAACTCCGCTAT |
| 353 | ACCTACGCATACCGCTTGGCGAGG | CCTCGCCAAGCGGTATGCGTAGGT |
| 354 | GATTACCTGAATGGCCAAGCGAGC | GCTCGCTTGGCCATTCAGGTAATC |
| 355 | CCTGTTAGCATCACGGCGCTTAGG | CCTAAGCGCCGTGATGCTAACAGG |
| 356 | CGGAATGATGCGCTCGACAACGCT | AGCGTTGTCGAGCGCATCATTCCG |
| 357 | TGAGAGAGGCGTTGGTTAAGGCAA | TTGCCTTAACCAACGCCTCTCTCA |
| 358 | AAGCAGGCGAAGGGATACTCCTCG | CGAGGAGTATCCCTTCGCCTGCTT |
| 359 | TCACGACAGACGGGCCGAGATTAC | GTAATCTCGGCCCGTCTGTCGTGA |
| 360 | AAGCAATTTGGCCTCGTTTTGTGA | TCACAAAACGAGGCCAAATTGCTT |
| 361 | GCTGGTTGCGGTAGGATCGCATAT | ATATGCGATCCTACCGCAACCAGC |
| 362 | TTGTGAATCCGTTCTGTCCCCGAC | GTCGGGGACAGAACGGATTCACAA |
| 363 | TGGGCTCCTCTGAGGCGAGATGGC | GCCATCTCGCCTCAGAGGAGCCCA |
| 364 | GGATAGAGTGAATCGACCGGCAAC | GTTGCCGGTCGATTCACTCTATCC |
| 365 | TGCACCGAACGTGCACGAGTAATT | AATTACTCGTGCACGTTCGGTGCA |
| 366 | GCCAGTATTCTCGGGTGTTGGACG | CGTCCAACACCCGAGAATACTGGC |
| 367 | TCGCTACCTAAGACCGGGCCATAC | GTATGGCCCGGTCTTAGGTAGCGA |
| 368 | TGGCATTGACGAGCAGCAGTCAGT | ACTGACTGCTGCTCGTCAATGCCA |
| 369 | CGCGTCCCAGCGCCCTTGGAGTAT | ATACTCCAAGGGCGCTGGGACGCG |
| 370 | ATGAAGCCTACCGGGCGACTTCGT | ACGAAGTCGCCCGGTAGGCTTCAT |
| 371 | CCAGACAGATGGCCTGGAACCATG | CATGGTTCCAGGCCATCTGTCTGG |
| 372 | TGGCGTGGGACCATCTCAAAGCTA | TAGCTTTGAGATGGTCCCACGCCA |
| 373 | CCGCATGGGAACACGTGTCAAGGT | ACCTTGACACGTGTTCCCATGCGG |
| 374 | GCCCACTCGTCAGCTGGACGTAAT | ATTACGTCCAGCTGACGAGTGGGC |
| 375 | ATTACGGTCGTGATCCAGAAAGCG | CGCTTTCTGGATCACGACCGTAAT |
| 376 | TGCGAGGTGAGCACCTACGAGAGA | TCTCTCGTAGGTGCTCACCTCGCA |
| 377 | GGGCCGCATTCTTGATGTCCATTC | GAATGGACATCAAGAATGCGGCCC |
| 378 | CCTCGGATGTGGGCTCTCGCCTAG | CTAGGCGAGAGCCCACATCCGAGG |
| 379 | TAGGCATGTTGGCGTGAGCGCTAT | ATAGCGCTCACGCCAACATGCCTA |
| 380 | CGATACGAACGAGGATGTCCGCCT | AGGCGGACATCCTCGTTCGTATCG |
| 381 | TACGCCGGTTAGCACGGTGCGCTA | TAGCGCACCGTGCTAACCGGCGTA |
| 382 | CATACGATGTCCGGGCCGTGTCGC | GCGACACGGCCCGGACATCGTATG |
| 383 | ATCCGCAGTTGTATGGCGCGTTAT | ATAACGCGCCATAGAACTGCGGAT |
| 384 | GGGTAAGGGACAAAGATGGGATGG | CCATCCCATCTTTGTCCCTTACCC |
| 385 | ATTGGAGTGTTTTGGTGAATCCGC | GCGGATTCACCAAAACACTCCAAT |
| 386 | GAACCGAGCCAACGTATGGACACG | CGTGTCCATACGTTGGCTCGGTTC |
| 387 | GCCGTCAAGCTTAAGGTTTTGGGC | GCCCAAAACCTTAAGCTTGACGGC |
| 388 | ACCTGCTTTTGGGTGGGTGATATG | CATATCACCCACCCAAAAGCAGGT |
| 389 | AATCGTGGGCGCAGCAAACGTATA | TATACGTTTGCTGCGCCCACGATT |
| 390 | GTCGCCGGATTGCTCAGTATAAGC | GCTTATACTGAGCAATCCGGCGAC |
| 391 | ACCCGTCGATGCTTCCTCCTCAGA | TCTGAGGAGGAAGCATCGACGGGT |
| 392 | ATCCGGGTGGGCGATACAAGAGAT | ATCTCTTGTATCGCCCACCCGGAT |
| 393 | TTCCGCATGAGTCAGCTTTGAAAA | TTTTCAAAGCTGACTCATGCGGAA |
| 394 | GCAAAGTCCCACTGGCAAGCCGAT | ATCGGCTTGCCAGTGGGACTTTGC |
| 395 | CGACCTCGGCTTCATCGTACACAT | ATGTGTACGATGAAGCCGAGGTCG |
| 396 | CTCATGAGCGCAGTTGTGCGTGAG | CTCACGCACAACTGCGCTCATGAG |
| 397 | CAGATGAAGGATCCACGGCCGGAG | CTCCGGCCGTGGATCCTTCATCTG |
| 398 | TCAAAGGCTCTTGGATACAGCCGT | ACGGCTGTATCCAAGAGCCTTTGA |
| 399 | TCCGCTAATTTCCAATCAGGGCTC | GAGCCCTGATTGGAAATTAGCGGA |
| 8 | CCGTTTGCGGTCGTCCTTGCTCAA | TTGAGCAAGGACGACCGCAAACGG |
| 9 | TTCGCTTTCGTGGCTGCACTTCAA | TTGAAGTGCAGCCACGAAAGCGAA |
| 402 | CTTAGTTGGGGCGCGGTATCCAGA | TCTGGATACCGCGCCCCAACTAAG |
| 403 | GCTCTAATGCCGTGGAGTCGGAAC | GTTCCGACTCCACGGCATTAGAGC |
| 404 | CCGATTACAAATTGACTGACCGCA | TGCGGTCAGTCAATTTGTAATCGG |
| 405 | AGACGTACGTGAGCCTCCCGTGTC | GACACGGGAGGCTCACGTACGTCT |
| 406 | AATGGAGCGATACGATCCAACGCA | TGCGTTGGATCGTATCGCTCCATT |
| 407 | GGAGGCGCTGTACTGATAGGCGTA | TACGCCTATCAGTACAGCGCCTCC |
| 408 | TGTTTTTGAATTGACCACACGGGA | TCCCGTGTGGTCAATTCAAAAACA |
| 409 | CATGTCTGGATGCGCTCAATGAAG | CTTCATTGAGCGCATCCAGACATG |
| 410 | GCCCGCTAATCCGACACCCAGTTT | AAACTGGGTGTCGGATTAGCGGGC |
| 411 | CCATTGACAGGAGAGCCATGAGCC | GGCTCATGGCTCTCCTGTCAATGG |
| 412 | GAATCACCGAATCACCGACTCGTT | AACGAGTCGGTGATTCGGTGATTC |
| 413 | AACCAGCCGCAGTAGCTTACGTCG | CGACGTAAGCTACTGCGGCTGGTT |
| 414 | TTTTCTGAGGGACACGCGGGCGTT | AACGCCCGCGTGTCCCTCAGAAAA |
| 415 | GGTGCTCCGTTTGATCGATCCTCC | GGAGGATCGATCAAACGGAGCACC |
| 416 | CCGCTTAGGCCATACTCTGAGCCA | TGGCTCAGAGTATGGCCTAAGCGG |
| 417 | TAAGACATACCGACGCCCTTGCCT | AGGCAAGGGCGTCGGTATGTCTTA |
| 418 | GTTCCCGACGCCAGTCATTGAGAC | GTCTCAATGACTGGCGTCGGGAAC |
| 419 | TAAAAGTTTCGCGGAGGTCGGGCT | AGCCCGACCTCCGCGAAACTTTTA |
| 420 | CGGTCCAGACGAGCTGAGTTCGGC | GCCGAACTCAGCTCGTCTGGACCG |
| 421 | CGGCGTAGCGGCTACGGACTTAAA | TTTAAGTCCGTAGCCGCTACGCCG |
| 422 | GCTTGGATGCCCATGCGGCAAGGT | ACCTTGCCGCATGGGCATCCAAGC |
| 423 | AGCGGGATCCCAGAGTTTCGAAAA | TTTTCGAAACTCTGGGATCCCGCT |
| 424 | GAGCTTGAGAGCGAGGTCATCCTC | GAGGATGACCTCGCTCTCAAGCTC |
| 425 | GCATCGGCCGTTTTGACCATATTC | GAATATGGTCAAAACGGCCGATGC |
| 426 | CATAGCGCTGCACGTTTCGACCGC | GCGGTCGAAACGTGCAGCGCTATG |
| 427 | ACCCGACAACCACCAATTCAAAAA | TTTTTGAATTGGTGGTTGTCGGGT |
| 428 | GCGAACACTCATAAGAGCGCCCTG | CAGGGCGCTCTTATGAGTGTTCGC |
| 429 | CCGCCGAGTGTAGAGAGACTCCGA | TCGGAGTCTCTCTACACTCGGCGG |
| 430 | GACATCGGGAGCCGGAAACATGAG | CTCATGTTTCCGGCTCCCGATGTC |
| 431 | TCGTGTAGACTCGGCGACAGGCGT | ACGCCTGTCGCCGAGTCTACACGA |
| 432 | ATGCGCATATACTGACTGCGCAGG | CCTGCGCAGTCAGTATATGCGCAT |
| 433 | ACAAGCGAACCCGAGTTTTGATGA | TCATCAAAACTCGGGTTCGCTTGT |
| 434 | GCATGAGACTCCGCGAAGACATGT | ACATGTCTTCGCGGAGTCTCATGC |
| 435 | TCCTACATGTCGCGTCACGATCAC | GTGATCGTGACGCGACATGTAGGA |
| 436 | GACCGATCGCGAAGTCGTACACAT | ATGTGTACGACTTCGCGATCGGTC |
| 437 | GTCGCCAGGACTGGGCCGATGTGA | TCACATCGGCCCAGTCCTGGCGAC |
| 438 | ACCGATAAGACTTGCATCCGAACG | CGTTCGGATGCAAGTCTTATCGGT |
| 439 | TCCATAACCAGTCCGAAGTGCCGG | CCGGCACTTCGGACTGGTTATGGA |
| 440 | ACGCGCCCTGCATCTCGTATTTAA | TTAAATACGAGATGCAGGGCGCGT |
| 441 | AGACCGCATCAATTGGCGCGTACC | GGTACGCGCCAATTGATGCGGTCT |
| 442 | AGAGGCTTGGCAAGTAGGGACCCT | AGGGTCCCTACTTGCCAAGCCTCT |
| 443 | GCAATGGACGCCAGACGATACCGG | CCGGTATCGTCTGGCGTCCATTGC |
| 444 | GCTGGACTTAGTCGTGTTCGGCGG | CCGCCGAACACGACTAAGTCCAGC |
| 445 | AGGCATCGTGCCGGATTGCTCCCT | AGGGAGCAATCCGGCACGATGCCT |
| 446 | TGCGCATGTCGACGTTGAACAAAG | CTTTGTTCAACGTCGACATGCGCA |
| 447 | TTCGGGTCACATCCGATGCCATAC | GTATGGCATCGGATGTGACCCGAA |
| 448 | ACCCATCGCCGGAAAGCGATGTTG | CAACATCGCTTTCCGGCGATGGGT |
| 449 | AAGCGCTGACTCGGCTAAGAATCA | TGATTCTTAGCCGAGTCAGCGCTT |
| 450 | ACTTCCAAGTCCTTGACCGTCCGA | TCGGACGGTCAAGGACTTGGAAGT |
| 451 | TCTCAATATTCCCGTAGTCGCCCA | TGGGCGACTACGGGAATATTGAGA |
| 452 | AACAGTTCCTCTTTTTCCTGGCGC | GCGCCAGGAAAAAGAGGAACTGTT |
| 453 | CGTCCTCCATGTTGTCACGAACAG | CTGTTCGTGACAACATGGAGGACG |
| 454 | TGCGCAGACCTACCTGTCTTTGCT | AGCAAAGACAGGTAGGTCTGCGCA |
| 455 | ATGGACGGCTTCGCAGTCCTCCTT | AAGGAGGACTGCGAAGCCGTCCAT |
| 456 | TGAACGCTTTCTATGGGCCACGTA | TACGTGGCCCATAGAAAGCGTTCA |
| 457 | TGAACCCTGCCGCGAGCGATAACC | GGTTATCGCTCGCGGCAGGGTTCA |
| 458 | GTTCTTGCGCGATGAATCAGGACC | GGTCCTGATTCATCGCGCAAGAAC |
| 459 | AGGGTACGTGTCGCAGCTTCGCGT | ACGCGAAGCTGCGACACGTACCCT |
| 460 | ACCCTTGCTCCGCCATGTCTCTCA | TGAGAGACATGGCGGAGCAAGGGT |
| 461 | GGGACAAGGATTGAAGCTGGCGTC | GACGCCAGCTTCAATCCTTGTCCC |
| 462 | TGTCGTTGCTCCCGAGTACCATTG | CAATGGTACTCGGGAGCAACGACA |
| 463 | GTTGTCCGAGACGTTTGTGTCAGC | GCTGACACAAACGTCTCGGACAAC |
| 464 | GCTGGTGAACACTCACGAACCGCT | AGCGGTTCGTGAGTGTTCACCAGC |
| 465 | GCAGACAGGGCAAATCGGTGCAAA | TTTGCACCGATTTGCCCTGTCTGC |
| 466 | CCCATCACAACGAGTGGCGACTTT | AAAGTCGCCACTCGTTGTGATGGG |
| 467 | GCTTCTACAGCTGGCGTGCTAGCG | CGCTAGCACGCCAGCTGTAGAAGC |
| 468 | GAATGTGTGCCGACCATTCTAGCC | GGCTAGAATGGTCGGCACACATTC |
| 469 | CCAGCGGAAGTTAGAGCTCTGTGG | CCACAGAGCTCTAACTTCCGCTGG |
| 470 | TTTTTACCGACCACTCCATGTCGG | CCGACATGGAGTGGTCGGTAAAAA |
| 471 | GCGGCTATGTGATGACGGCCTAGC | GCTAGGCCGTCATCACATAGCCGC |
| 472 | AGTACACGGGCGTGTTAGCGCTCC | GGAGCGCTAACACGCCCGTGTACT |
| 473 | TCCTGTGTGGTGGCGCACTCCCAC | GTGGGAGTGCGCCACCACACAGGA |
| 474 | CCAACTAACCAATCGCGCGGATGA | TCATCCGCGCGATTGGTTAGTTGG |
| 475 | AGTGAGTGACCAAGGCAGGAGCAA | TTGCTCCTGCCTTGGTCACTCACT |
| 476 | CATCTTTCGCGGAGTTTATTGCGG | CCGCAATAAACTCCGCGAAAGATG |
| 477 | CTTCGTCCGGTTAGTGCGACAGCA | TGCTGTCGCACTAACCGGACGAAG |
| 478 | CTCACGAAAACGTGGGCCCGAAAT | ATTTCGGGCCCACGTTTTCGTGAG |
| 479 | CGCAGCAGCTGAACTCTAGCATTG | CAATGCTAGAGTTCAGCTGCTGCG |
| 480 | AGGAGACATACGCCCAAATGGTGC | GCACCATTTGGGCGTATGTCTCCT |
| 481 | ATTGAGAACTCGTGCGGGAGTTTG | CAAACTCCCGCACGAGTTCTCAAT |
| 482 | CTCTTTGTAGGCCCAGGAGGAGCA | TGCTCCTCCTGGGCCTACAAAGAG |
| 483 | GCCGCAGGGTCGATAATTGGTCTA | TAGACCAATTATCGACCCTGCGGC |
| 484 | AAACGCCGCCCTGAGACTATTGGG | CCCAATAGTCTCAGGGCGGCGTTT |
| 485 | CTGAGTTGCCTGGAACGTTGGACT | AGTCCAACGTTCCAGGCAACTCAG |
| 486 | CGGATGGGTTGCAGAGTATGGGAT | ATCCCATACTCTGCAACCCATCCG |
| 487 | CTGACCTTTGGGGGTTAGTGCGGT | ACCGCACTAACCCCCAAAGGTCAG |
| 488 | GGAAATGAGAACCTTACCCCAGCG | CGCTGGGGTAAGGTTCTCATTTCC |
| 489 | AACGCATCGTCCGTCAACTCATCA | TGATGAGTTGACGGACGATGCGTT |
| 490 | TGGAGAGAGACTTCGGCCATTGTT | AACAATGGCCGAAGTCTCTCTCCA |
| 491 | TTGCGCTCATTGGATCTTGTCAGG | CCTGACAAGATCCAATGAGCGCAA |
| 492 | AGCGCGTTAAAGCACGGCAACATT | AATGTTGCCGTGCTTTAACGCGCT |
| 493 | AGCCAGTAAACTGTGGGCGGCTGT | ACAGCCGCCCACAGTTTACTGGCT |
| 494 | CGACTGATGTGCAACCAGCAGCTG | CAGCTGCTGGTTGCACATCAGTCG |
| 495 | GGTTGCTCATACGACGAGCGAGTG | CACTCGCTCGTCGTATGAGCAACC |
| 10 | GTCCAACGCGCAACTCCGATTCAA | TTGAATCGGAGTTGCGCGTTGGAC |
| 11 | TTGCCGCACCGTCCGTCATCTCAA | TTGAGATGACGGACGGTGCGGCAA |
| 498 | AGAACCTCCGCGCCTCCGTAGTAG | CTACTACGGAGGCGCGGAGGTTCT |
| 499 | AAAGGAGCTTTCGCCCAACGTACC | GGTACGTTGGGCGAAAGCTCCTTT |
| 500 | AGTGATTGTGCCACTCCACAGCTC | GAGCTGTGGAGTGGCACAATCACT |
| 501 | GCGATCGTCGAGGGTTGAGCTGAA | TTCAGCTCAACCCTCGACGATCGC |
| 502 | GGGAGACAGCCATTATGGTCCTCG | CGAGGACCATAATGGCTGTCTCCC |
| 503 | GAGACGCTGTCACTCCGGCAGAAC | GTTCTGCCGGAGTGACAGCGTCTC |
| 504 | CCACCGGTCGCTTAAGATGCACTT | AAGTGCATCTTAAGCGACCGGTGG |
| 505 | CGGCATAACGTCCAGTCCTGGGAC | GTCCCAGGACTGGACGTTATGCCG |
| 506 | AAGCGGAACGGGTTATACCGAGGT | ACCTCGGTATAACCCGTTCCGCTT |
| 507 | TGCACACTAGGTCCGTCGCTTGAT | ATCAAGCGACGGACCTAGTGTGCA |
| 508 | AGGGAACCGCGTTCAAACTCAGTT | AACTGAGTTTGAACGCGGTTCCCT |
| 509 | GAATTACAACCACCCGCTCGTGTT | AACACGAGCGGGTGGTTGTAATTC |
| 510 | TTCAGTGCTCACGAAGCATGGATT | AATCCATGCTTCGTGAGCACTGAA |
| 511 | TTAGTTTGGCGTTGGGACTTCACC | GGTGAAGTCCCAACGCCAAACTAA |
| 512 | AATGCGACCTCGACGAGCCTCATA | TATGAGGCTCGTCGAGGTCGCATT |
| 513 | CCGAAACCGTTAACGTGGCGCACA | TGTGCGCCACGTTAACGGTTTCGG |
| 514 | TAAAGTAACAAGGCGACCTCCCGC | GCGGGAGGTCGCCTTGTTACTTTA |
| 515 | TAATGATTTTAGTCGCGGGGTGGG | CCCACCCCGCGACTAAAATCATTA |
| 516 | GGCTACTCTAAGTGCCCGCTCAGG | CCTGAGCGGGCACTTAGAGTAGCC |
| 517 | TGGCGGACGACTCAATATCTCACG | CGTGAGATATTGAGTCGTCCGCCA |
| 518 | GGGCGTTAGGCGTAATAGACCGTC | GACGGTCTATTACGCCTAACGCCC |
| 519 | GCCACCTTTAGACGGCGGCTCTAG | CTAGAGCCGCCGTCTAAAGGTGGC |
| 520 | GAGATGTGTAAACGTGCAGGCACC | GGTGCCTGCACGTTTACACATCTC |
| 521 | TAGCTCGTGGCCCTCCAAGCGTGT | ACACGCTTGGAGGGCCACGAGCTA |
| 522 | GTGTCGGCGCTATTTGGCCTTACC | GGTAAGGCCAAATAGCGCCGACAC |
| 523 | CCAGGGAAGCAACTGGTTGCCATT | AATGGCAACCAGTTGCTTCCCTGG |
| 524 | TTCCGAAACTAAGCCAGAACCGCT | AGCGGTTCTGGCTTAGTTTCGGAA |
| 525 | GCAAACCCGGTAACCCGAGAGTTC | GAACTCTCGGGTTACCGGGTTTGC |
| 526 | GCAAATGGCGTCATGCACGAACGT | ACGTTCGTGCATGACGCCATTTGC |
| 527 | AGTACTTTCGCGCCCAGTTTAGGG | CCCTAAACTGGGCGCGAAAGTACT |
| 528 | AAGATCTGCGAGGCATCCCGGCTT | AAGCCGGGATGCCTCGCAGATCTT |
| 529 | GCAAGTGTATCGCACAGTGCGATT | AATCGCACTGTGCGATACACTTGC |
| 530 | CCGACAAGGCCTCAATTCATTCTG | CAGAATGAATTGAGGCCTTGTCGG |
| 531 | GTCTCGTCTCAACTTTAAGGCGCG | CGCGCCTTAAAGTTGAGACGAGAC |
| 532 | ATCCAGAGATCCGTTTTGCAGCGT | ACGCTGCAAAACGGATCTCTGGAT |
| 533 | GTCACCAGGAGGGAAGTTTCACCC | GGGTGAAACTTCCCTCCTGGTGAC |
| 534 | TTCCGTCAGGCGGATCAACGGAAT | ATTCCGTTGATCCGCCTGACGGAA |
| 535 | ATGCCGGACACGCATTACACAGGC | GCCTGTGTAATGCGTGTCCGGCAT |
| 536 | TGGGCCGCTTGGCGCTTTCATAGA | TCTATGAAAGCGCCAAGCGGCCCA |
| 537 | CCTAGCGCGAGCTTTACTGACCAG | CTGGTCAGTAAAGCTCGCGCTAGG |
| 538 | TTGGCCAGGAATATGGTCTCGAGA | TCTCGAGACCATATTCCTGGCCAA |
| 539 | GTCTGCGGCCGACTTGCTATGCAT | ATGCATAGCAAGTCGGCCGCAGAC |
| 540 | AACTTGCTCATTCTCAAGCCGACG | CGTCGGCTTGAGAATGAGCAAGTT |
| 541 | ACGTCAGCGATTGTGGCGAAATAT | ATATTTCGCCACAATCGCTGACGT |
| 542 | ACGGCCTGCGTCAGCACATGCATC | GATGCATGTGCTGACGCAGGCCGT |
| 543 | ATACCTCCGCAGAACCATTCCGTT | AACGGAATGGTTCTGCGGAGGTAT |
| 544 | AGTTCGCGGTCCCACGATTCACTT | AAGTGAATCGTGGGACCGCGAACT |
| 545 | TGCTCAATTTGTGCAGAAAACGCC | GGCGTTTTCTGCACAAATTGAGCA |
| 546 | TTATCGCGAGAGACGACCGTGTCC | GGACACGGTCGTCTCTCGCGATAA |
| 547 | GACGCGACGTGAGTAGTGGAAGCG | CGCTTCCACTACTCACGTCGCGTC |
| 548 | ATGGTAGGGGCATTGGGCTTTCCT | AGGAAAGCCCAATGCCCCTACCAT |
| 549 | CCAAATATAGCCGCGCGGAGACAT | ATGTCTCCGCGCGGCTATATTTGG |
| 550 | GCAAACCCTGATTGAATCGTGCCC | GGGCACGATTCAATCAGGGTTTGC |
| 551 | TAGCGTCTTGCGTGAAACCATGGG | CCCATGGTTTCACGCAAGACGCTA |
| 552 | CCACCCCGACAGCGCTGGACTCTT | AAGAGTCCAGCGCTGTCGGGGTGG |
| 553 | ACGAGCACTGAAGGCTGCTTTACG | CGTAAAGCAGCCTTCAGTGCTCGT |
| 554 | CATATCAGCGTCGTCTAGCTCGCG | CGCGAGCTAGACGACGCTGATATG |
| 555 | TGATCCCGGACCGGCTAGACTAAT | ATTAGTCTAGCCGGTCCGGGATCA |
| 556 | GGCCCCGACACTACAGGGTAATCA | TGATTACCCTGTAGTGTCGGGGCC |
| 557 | GGCTCCAGGGCGAGATTATGAATG | CATTCATAATCTCGCCCTGGAGCC |
| 558 | CAAAATCCGATGGGCGGAAAATTA | TAATTTTCCGCCCATCGGATTTTG |
| 559 | CACAGGCGCATAGGGAGCAAGCTA | TAGCTTGCTCCCTATGCGCCTGTG |
| 560 | TAGCTATTGCCCCGATGGGCTACT | AGTAGCCCATCGGGGCAATAGCTA |
| 561 | TGGTACGCGGTCCATAGCAAGTCG | CGACTTGCTATGGACCGCGTACCA |
| 562 | GACGCTGTGGCTCGGAAACTGTTC | GAACAGTTTCCGAGCCACAGCGTC |
| 563 | CCTGGGTTCGCCGCGTGGTAACTG | CAGTTACCACGCGGCGAACCCAGG |
| 564 | TTCCCGCGTAGCCCAACAGCTATA | TATAGCTGTTGGGCTACGCGGGAA |
| 565 | TTCGCGGATTGCTGCCGCATAACA | TGTTATGCGGCAGCAATCCGCGAA |
| 566 | AAAAATGGCACCGAAGTTGAGGCA | TGCCTCAACTTCGGTGCCATTTTT |
| 567 | CATTCCGCGCGAGTTGAAATCCAG | CTGGATTTCAACTCGCGCGGAATG |
| 568 | ACGCACGTTTTTTGGCACGGTTAA | TTAACCGTGCCAAAAAACGTGCGT |
| 569 | TGTCCATGACGTCGTTTCTCTGGT | ACCAGAGAAACGACGTCATGGACA |
| 570 | TCTCAGTCGGACTCGTATGCCAGA | TCTGGCATACGAGTCCGACTGAGA |
| 571 | CTCCAAACGCACACATCAAGCATC | GATGCTTGATGTGTGCGTTTGGAG |
| 572 | TTCAACCAAGCGGGGTGTTCGTGA | TCACGAACACCCCGCTTGGTTGAA |
| 573 | GGTGTCGGAGGGTGGTGACCTCGA | TCGAGGTCACCACCCTCCGACACC |
| 574 | AGCGCTTTTGGTCATGATTTGCAA | TTGCAAATCATGACCAAAAGCGCT |
| 575 | CCGAGGACTTACGTCTGCCCAGGA | TCCTGGGCAGACGTAAGTCCTCGG |
| 576 | GCCCAATCCAGTTCTTATGCGCCC | GGGCGCATAAGAACTGGATTGGGC |
| 577 | CGGGTTAACCCACGCAAGTTATGA | TCATAACTTGCGTGGGTTAACCCG |
| 578 | TGATTAGCGCTCAATACACGCGTG | CACGCGTGTATTGAGCGCTAATCA |
| 579 | AAGGGCAGACCTTTGGTTCGACTG | CAGTCGAACCAAAGGTCTGCCCTT |
| 580 | GCGCCACAAGATTCACATGTCATT | AATGACATGTGAATCTTGTGGCGC |
| 581 | GCCATGTTCAAGGGCCTTTCGAAG | CTTCGAAAGGCCCTTGAACATGGC |
| 582 | CGCGGTGTTTTGTCTAGGTGCCGG | CCGGCACCTAGACAAAACACCGCG |
| 583 | CAACATTGTGGTGGCACTCCATCC | GGATGGAGTGCCACCACAATGTTG |
| 584 | CGATACGCGCCGGTTTGTTAAATC | GATTTAACAAACCGGCGCGTATCG |
| 585 | GGCTATAAACGTGCGGACTGCTCC | GGAGCAGTCCGCACGTTTATAGCC |
| 586 | TGGGTAAATCACTATTGCGCGGTT | AACCGCGCAATAGTGATTTACCCA |
| 587 | GTCTTCATCGGCCCGCGCAAGCTA | TAGCTTGCGCGGGCCGATGAAGAC |
| 588 | GCGACACACCCTGTACTCTGATGC | GCATCAGAGTACAGGGTGTGTCGC |
| 589 | GTAGCAGGGTCCGCAAGACCAAGC | GCTTGGTCTTGCGGACCCTGCTAC |
| 590 | TCGCCAACGCAGGGTAACTGCCAT | ATGGCAGTTACCCTGCGTTGGCGA |
| 591 | ACTCCGAAGCTTCGAGCGGCACGA | TCGTGCCGCTCGAAGCTTCGGAGT |
| 12 | CATCGTCCCTTTCGATGGGATCAA | TTGATCCCATCGAAAGGGACGATG |
| 13 | GCACGGGAGCTGACGACGTGTCAA | TTGACACGTCGTCAGCTCCCGTGC |
| 594 | ATCATCCCACGGCAGAGTGAAGAG | CTCTTCACTCTGCCGTGGGATGAT |
| 595 | CGCTGGACTGGCCTATCCGAGTCG | CGACTCGGATAGGCCAGTCCAGCG |
| 596 | CGGTCTCAGCAACACTGTCGCAAA | TTTGCGACAGTGTTGCTGAGACCG |
| 597 | CGAACGTTCTCCGATGTAATGGCC | GGCCATTACATCGGAGAACGTTCG |
| 598 | ATACCGTGCGACAAGCCCCTCTGA | TCAGAGGGGCTTGTCGCACGGTAT |
| 599 | AGCTCATTCCCGAGACGGAACACC | GGTGTTCCGTCTCGGGAATGAGCT |
| 600 | TTTCATGCGGCCGTTGCAAATCAT | ATGATTTGCAACGGCCGCATGAAA |
| 601 | ACTCGAACGGACGTTCAATTCCCA | TGGGAATTGAACGTCCGTTCGAGT |
| 602 | CTGCATGGTGTGGGTGAGACTCCC | GGGAGTCTCACCCACACCATGCAG |
| 603 | CCGCGAGTGTGGATGGCGTGTTGA | TCAACACGCCATCCACACTCGCGG |
| 604 | AATGTGTCGGTCCTAAGCCGGGTG | CACCCGGCTTAGGACCGACACATT |
| 605 | TAAGACGAGCCTGCACAGCTTGCG | CGCAAGCTGTGCAGGCTCGTCTTA |
| 606 | GGCGTGGGAGGATAAGACGATGTC | GACATCGTCTTATCCTCCCACGCC |
| 607 | TGCTCCATGTTAGGAACGCACCAC | GTGGTGCGTTCCTAACATGGAGCA |
| 608 | CGGTGTTGGTCGGACTGACGACTG | CAGTCGTCAGTCCGACCAACACCG |
| 609 | CCGCGCGTATCTATCAGATCTGGG | CCCAGATCTGATAGATACGCGCGG |
| 610 | AAAGCATGCTCCACCTGGAGCGAG | CTCGCTCCAGGTGGAGCATGCTTT |
| 611 | ACTTGCATCGCTGGGTAGATCCGG | CCGGATCTACCCAGCGATGCAAGT |
| 612 | TGCTTACGCAGTGGATTGGTCAGA | TCTGACCAATCCACTGCGTAAGCA |
| 613 | ATGCAGATGAACAAATCGCCGAAT | ATTCGGCGATTTGTTCATCTGCAT |
| 614 | GCAATTCTGGGCCATGTATTCGTC | GACGAATACATGGCCCAGAATTGC |
| 615 | AGGGTTCCTTACGCGTCGACATGG | CCATGTCGACGCGTAAGGAACCCT |
| 616 | GTGGAGCTAATCGCGAGCCTCAGA | TCTGAGGCTCGCGATTAGCTCCAC |
| 617 | TCGTAGTCTCACCGGCAATGATCC | GGATCATTGCCGGTGAGACTACGA |
| 618 | TTATAGCAGTGCGCCAATGCTTCG | CGAAGCATTGGCGCACTGCTATAA |
| 619 | CGAACAGTGCTGTCCGTCGCTCAA | TTGAGCGACGGACAGCACTGTTCG |
| 620 | TCCGCGTGGACTGTTAGACGCTAT | ATAGCGTCTAACAGTCCACGCGGA |
| 621 | CATTAGCCCGCTGTCGGTAACTGT | ACAGTTACCGACAGCGGGCTAATG |
| 622 | GGAAAGAAACTCAGACGCGCAATG | CATTGCGCGTCTGAGTTTCTTTCC |
| 623 | CGACTCGCTGGACAGGAGAATCGT | ACGATTCTCCTGTCCAGCGAGTCG |
| 624 | CATGATCCTCTGTTTCACCCGCGG | CCGCGGGTGAAACAGAGGATCATG |
| 625 | GGCGTAGCGCTCTAAAAGCTTCGG | CCGAAGCTTTTAGAGCGCTACGCC |
| 626 | AGTGATGCCATCAGGCCCGTATAC | GTATACGGGCCTGATGGCATCACT |
| 627 | TATGGAAAGGGCAACAGCGCTATC | GATAGCGCTGTTGCCCTTTCCATA |
| 628 | CTGTGGTTGATGGAGGATCCACAC | GTGTGGATCCTCCATCAACCACAG |
| 629 | ACTCGCTGGAATTTGCGCTGACAC | GTGTCAGCGCAAATTCCAGCGAGT |
| 630 | CAGGCCCGAACCACGCGGTTACAG | CTGTAACCGCGTGGTTCGGGCCTG |
| 631 | GGCGCAATGGGCGCATAAATACTA | TAGTATTTATGCGCCCATTGCGCC |
| 632 | GGTCAATTCGCGCTACATGCCCTA | TAGGGCATGTAGCGCGAATTGACC |
| 633 | GATGGTGGACTGGAGCCCTTCCGC | GCGGAAGGGCTCCAGTCCACCATC |
| 634 | CCGCGCATAGCGCAATAGGGGAGA | TCTCCCCTATTGCGCTATGCGCGG |
| 635 | TCTTCTGGCTGTCCGGCACCCGAA | TTCGGGTGCCGGACAGCCAGAAGA |
| 636 | GCGTTCGCAATTCACGGGCCCTTA | TAAGGGCCCGTGAATTGCGAACGC |
| 637 | TCGTTTCGGCCTTGGAGAGTATCG | CGATACTCTCCAAGGCCGAAACGA |
| 638 | AGGTGCAAGTGCAAGGCGAGAGGC | GCCTCTCGCCTTGCACTTGCACCT |
| 639 | CGCCAGTTTCGATGGCTGACGTTT | AAACGTCAGCCATCGAAACTGGCG |
| 640 | GCTTTACCGCCGATCCCAGATATC | GATATCTGGGATCGGCGGTAAAGC |
| 641 | GTGCTTGACGAAGAGGCGAAATGT | ACATTTCGCCTCTTCGTCAAGCAC |
| 642 | CAGTCCGTGCGCTTCATGTCCTCA | TGAGGACATGAAGCGCACGGACTG |
| 643 | TACGCGTAAGAGCCTACCCTCGCG | CGCGAGGGTAGGCTCTTACGCGTA |
| 644 | GGCGAGTCTTGTGGGGACATGTGT | ACACATGTCCCCACAAGACTCGCC |
| 645 | CCAAAGCGAAGCGAGCGTGTCTAT | ATAGACACGCTCGCTTCGCTTTGG |
| 646 | GCCGTAGGTTGCTCTTCACCGAAC | GTTCGGTGAAGAGCAACCTACGGC |
| 647 | AAATCCGCGATGTGCCGTGAGGCT | AGCCTCACGGCACATCGCGGATTT |
| 648 | GGCTTCGCACCCGTACCAATTTAG | CTAAATTGGTACGGGTGCGAAGCC |
| 649 | TGTAGAGTCCCACGTAGCCGGCAT | ATGCCGGCTACGTGGGACTCTACA |
| 650 | CACTAGTCTGGGGCAAGGTGCATT | AATGCACCTTGCCCCAGACTAGTG |
| 651 | TGTACTCGGCAGGCGCAATAGATT | AATCTATTGCGCCTGCCGAGTACA |
| 652 | AACGGGTATCGGAAGCGTAAAAGC | GCTTTTACGCTTCCGATACCCGTT |
| 653 | CGGACTGCCCGTTTGCAAGTTGAG | CTCAACTTGCAAACGGGCAGTCCG |
| 654 | ATCGTTCAGCACTGGAGCCCGTAA | TTACGGGCTCCAGTGCTGAACGAT |
| 655 | ATGCATCGAACTAGTCGTGACGGC | GCCGTCACGACTAGTTCGATGCAT |
| 656 | TTCCAGGCATTAAGGAGAGGGAGC | GCTCCCTCTCCTTAATGCCTGGAA |
| 657 | GTGCGACATCTACTCCACGATCCC | GGGATCGTGGAGTAGATGTCGCAC |
| 658 | CTCATCGTCCTAACACGAGAGCCC | GGGCTCTCGTGTTAGGACGATGAG |
| 659 | AATGGCACTTCGGCGGTGATGCAA | TTGCATCACCGCCGAAGTGCCATT |
| 660 | CCGTGGGAGGGAATCCAACCGAGG | CCTCGGTTGGATTCCCTCCCACGG |
| 661 | AAATTCTCGTTGGTGACGGCTCAT | ATGAGCCGTCACCAACGAGAATTT |
| 662 | TTGCTCTTATCCTTGTCCTGGGCG | CGCCCAGGACAAGGATAAGAGCAA |
| 663 | TTAAGGATCAGGCGGAGCTTGCAG | CTGCAAGCTCCGCCTGATCCTTAA |
| 664 | CGCGACTAAGGTGCTGCAACTCGA | TCGAGTTGCAGCACCTTAGTCGCG |
| 665 | GCTCGATTTCACGGCCCGTTGTTC | GAACAACGGGCCGTGAAATCGAGC |
| 666 | AGCAGAGTGCGTTGCAGAGGCTAA | TTAGCCTCTGCAACGCACTCTGCT |
| 667 | TGGAGGTGAGGACGACGTGCACTA | TAGTGCACGTCGTCCTCACCTCCA |
| 668 | AACCGTTTAGGGTACATTCGCGGT | ACCGCGAATGTACCCTAAACGGTT |
| 669 | TATGATCGCTCGGCTCACAGTTTG | CAAACTGTGAGCCGAGCGATCATA |
| 670 | GACTTTTTGCGGAAACGTCATGGT | ACCATGACGTTTCCGCAAAAAGTC |
| 671 | TGTCGGTTATTCCACCTGCAAGGA | TCCTTGCAGGTGGAATAACCGACA |
| 672 | CTATGGTTTGCACTGCGCCGTCGA | TCGACGGCGCAGTGCAAACCATAG |
| 673 | AGCAGGGAAATTCAATCGTTCGCA | TGCGAACGATTGAATTTCCCTGCT |
| 674 | CCTAACCGAGCGCTTAGCATTTCC | GGAAATGCTAAGCGCTCGGTTAGG |
| 675 | CCCGACCCTAACTCGCATTGAATA | TATTCAATGCGAGTTAGGGTCGGG |
| 676 | TTGCTTAATGGTGACGCCACGGAT | ATCCGTGGCGTCACCATTAAGCAA |
| 677 | GATGCTCGCCGTGTTTAGTTCACG | CGTGAACTAAACACGGCGAGCATC |
| 678 | TCGGATGACGAGTTTCCATGACGG | CCGTCATGGAAACTCGTCATCCGA |
| 679 | ATGCGGTCTACTTTCTCGATCGGG | CCCGATCGAGAAAGTAGACCGCAT |
| 680 | TTGCGAGGCTAAGCACACGGTAAA | TTTACCGTGTGCTTAGCCTCGCAA |
| 681 | AACTTAATTACCGCCTCTGGCGCC | GGCGCCAGAGGCGGTAATTAAGTT |
| 682 | GTGACCGCGAACTTGTTCCGACAG | CTGTCGGAACAAGTTCGCGGTCAC |
| 683 | TGCGGATTACCGATTCGCTCTTAA | TTAAGAGCGAATCGGTAATCCGCA |
| 684 | TGATAGGGGGCCACGTTGATCAGA | TCTGATCAACGTGGCCCCCTATCA |
| 685 | TCGCTCCGTAGCGATTCATCGTAG | CTACGATGAATCGCTACGGAGCGA |
| 686 | TGTCAGCTGGTAGCCTCCGTTTGA | TCAAACGGAGGCTACCAGCTGACA |
| 687 | AGCGTCGCATGACGCTTACGGCAC | GTGCCGTAAGCGTCATGCGACGCT |
| 14 | AGACGCACCGCAACAGGCTGTCAA | TTGACAGCCTGTTGCGGTGCGTCT |
| 15 | CGTGTAGGGGTCCCGTGCTGTCAA | TTGACAGCACGGGACCCCTACACG |
| 690 | GTCGCATTCTGCACTGGCTTCGCC | GGCGAAGCCAGTGCAGAATGCGAC |
| 691 | TGATTAGGTGCGGTCCCGTAGTCC | GGACTACGGGACCGCACCTAATCA |
| 692 | AAGGGACCTTGGGTGACGGCGAGA | TCTCGCCGTCACCCAAGGTCCCTT |
| 693 | TCAAATGGCCACCGCGTGTCATTC | GAATGACACGCGGTGGCCATTTGA |
| 694 | CTCCGACGACCAATAAATAGCCGC | GCGGCTATTTATTGGTCGTCGGAG |
| 695 | GGCTATTCCCGTAGAGAGCGTCCA | TGGACGCTCTCTACGGGAATAGCC |
| 696 | TGGATAACCTCTCGGTCCATCCAC | GTGGATGGACCGAGAGGTTATCCA |
| 697 | GACCGCTGTACGGGAGTGTGCCTT | AAGGCACACTCCCGTACAGCGGTC |
| 698 | GCCACAGAGTTTTAGCAGGGACCC | GGGTCCCTGCTAAAACTCTGTGGC |
| 699 | CCCACGCTTTCCGACCACTGACCT | AGGTCAGTGGTCGGAAAGCGTGGG |
| 700 | CATTGACACAATGCGGGGACTGAT | ATCAGTCCCCGCATTGTGTCAATG |
| 701 | AGCCACTCGACAGGGTTCCAAAGC | GCTTTGGAACCCTGTCGAGTGGCT |
| 702 | CAGGATGAGCAAAGCGACTCTCCA | TGGAGAGTCGCTTTGCTCATCCTG |
| 703 | CAAGGTATGGTCTGGGGCCTAAGC | GCTTAGGCCCCAGACCATACCTTG |
| 704 | GGTGTTCGGCCTAAACTCTTTCGG | CCGAAAGAGTTTAGGCCGAACACC |
| 705 | TTTAGTCGGACCCTGTGGCAATTC | GAATTGCCACAGGGTCCGACTAAA |
| 706 | CACACGTTTCCGACCAGCCTGAAC | GTTCAGGCTGGTCGGAAACGTGTG |
| 707 | CTGGACGAACTGGCTTCCTCGTAC | GTACGAGGAAGCCAGTTCGTCCAG |
| 708 | TTCACAATCCGCCGAAAACTGACC | GGTCAGTTTTCGGCGGATTGTGAA |
| 709 | AACAGGATATCCGCGATCACGACA | TGTCGTGATCGCGGATATCCTGTT |
| 710 | TACGTCGGATCCATTGCGCCGAGT | ACTCGGCGCAATGGATCCGACGTA |
| 711 | CATGGATCTCTCGGTTTGATCGCC | GGCGATCAAACCGAGAGATCCATG |
| 712 | AGCCAGGCGCGTATATACGCTCGG | CCGAGCGTATATACGCGCCTGGCT |
| 713 | ATTTGGCACGTGTCGTGCCATGTT | AACATGGCACGACACGTGCCAAAT |
| 714 | CCGCGTTGCACCACTTTGAGGTGC | GCACCTCAAAGTGGTGCAACGCGG |
| 715 | TTGGACGTGACAAGCATGGCGCTC | GAGCGCCATGCTTGTCACGTCCAA |
| 716 | CTGAATCGCGCAAGTAAATGGGGG | CCCCCATTTACTTGCGCGATTCAG |
| 717 | GATAAGGTCCACCAGATTGCGCGC | GCGCGCAATCTGGTGGACCTTATC |
| 718 | CTAACAATTGCCAACCGGGACGGC | GCCGTCCCGGTTGGCAATTGTTAG |
| 719 | GGTAACCTGGGTGCTTGCAGGTTA | TAACCTGCAAGCACCCAGGTTACC |
| 720 | ATCGGAGCCACCATTCGCATTGGG | CCCAATGCGAATGGTGGCTCCGAT |
| 721 | GTGAACTGGCTTGCCCCAGGATTA | TAATCCTGGGGCAAGCCAGTTCAC |
| 722 | AGGCGATAGCATGGTCCCATATGA | TCATATGGGACCATGCTATCGCCT |
| 723 | AACGGTATCGTGGCTAATGCACGA | TCGTGCATTAGCCACGATACCGTT |
| 724 | AGTAGTGGTCCTCCAGATCGGCAA | TTGCCGATCTGGAGGACCACTACT |
| 725 | CCGTTGAATTGGACGGGAGGTTAG | CTAACCTCCCGTCCAATTCAACGG |
| 726 | GCATAAGTGCGGCATCGCGAAGGG | CCCTTCGCGATGCCGCACTTATGC |
| 727 | CGACAAGATGCAGCTGCTACATGC | GCATGTAGCAGCTGCATCTTGTCG |
| 728 | TCGCAGTGATTCCCGACCGATAAG | CTTATCGGTCGGGAATCACTGCGA |
| 729 | CAAGGCGAGTCCACTCGAGGGGAC | GTCCCCTCGAGTGGACTCGCCTTG |
| 730 | GCAACTTGCACGGCATAAGTGGCC | GGCCACTTATGCCGTGCAAGTTGC |
| 731 | TCCGAGCTTGACGTTCGCGACGTC | GACGTCGCGAACGTCAAGCTCGGA |
| 732 | AGCGCTGGGCTGTGCTGCCATCTC | GAGATGGCAGCACAGCCCAGCGCT |
| 733 | TTCATGTCGCTGAGTAACCCTCGC | GCGAGGGTTACTCAGCGACATGAA |
| 734 | CGAACCGCTAATGCCCATTGTCAG | CTGACAATGGGCATTAGCGGTTCG |
| 735 | CACGGAAGGTGGGACAAATCGCCG | CGGCGATTTGTCCCACCTTCCGTG |
| 736 | CACAGATGGAGACAAACGCGCCTT | AAGGCGCGTTTGTCTCCATCTGTG |
| 737 | TTTTCGCAACTCGCTCCATAACCC | GGGTTATGGAGCGAGTTGCGAAAA |
| 738 | ACGTTACGTTTCCGGCGCCTCTAA | TTAGAGGCGCCGGAAACGTAACGT |
| 739 | TATCGGATTGCGTGGGTTTCAATC | GATTGAAACCCACGCAATCCGATA |
| 740 | CTTCCACAATTGTCTGCGACGCAC | GTGCGTCGCAGACAATTGTGGAAG |
| 741 | TGCACAAAGGTATGGCTGTCCGGC | GCCGGACAGCCATACCTTTGTGCA |
| 742 | TCCGATGCCAGTCCCATCTTAAGA | TCTTAAGATGGGACTGGCATCGGA |
| 743 | CTGAAACCGTGCGAATCGAGGTGA | TCACCTCGATTCGCACGGTTTCAG |
| 744 | CGGTGTTCCGCGTGTCGAAAAAAT | ATTTTTTCGACACGCGGAACACCG |
| 745 | TCTAGCAGGCCTTTTGAATCGCCA | TGGCGATTCAAAAGGCCTGCTAGA |
| 746 | GAGTCACCTCTGAGACGGACGCCA | TGGCGTCCGTCTCAGAGGTGACTC |
| 747 | TCTTCTGTCATCCTGCAGCAGCAT | ATGCTGCTGCAGGATGACAGAAGA |
| 748 | GCGGATGAAACCTGAAAGGGGCCT | AGGCCCCTTTCAGGTTTCATCCGC |
| 749 | GGGGCCCCAAACTGGTATCAAGCC | GGCTTGATACCAGTTTGGGGCCCC |
| 750 | GCATTGGCTTCGGATTCTCCTACA | TGTAGGAGAATCCGAAGCCAATGC |
| 751 | AGGCGGCCCAACTGTGAGGTCTTG | CAAGACCTCACAGTTGGGCCGCCT |
| 752 | ACACCATGTGCTCCGCGCTGCAGT | ACTGCAGCGCGGAGCACATGGTGT |
| 753 | ACGATGAACATGAATCGGGAGTCG | CGACTCCCGATTCATGTTCATCGT |
| 754 | CTGCATCCCTGTAGCAGCGCTCCG | CGGAGCGCTGCTACAGGGATGCAG |
| 755 | GTGCCGTATTTCGACCTGTGCGTT | AACGCACAGGTCGAAATACGGCAC |
| 756 | GCAGTGCGCACTTCAGTTCAAAAG | CTTTTGAACTGAAGTGCGCACTGC |
| 757 | GCGATTTTAAGCGATGCCTTGACG | CGTCAAGGCATCGCTTAAAATCGC |
| 758 | TAGGTGACCTAGGCTTGCTTGCGG | CCGCAAGCAAGCCTAGGTCACCTA |
| 759 | CTGGATACCTTGCCTGTGCGGCGC | GCGCCGCACAGGCAAGGTATCCAG |
| 760 | CCCCTTACGGCTCGTCGTCTATGC | GCATAGACGACGAGCCGTAAGGGG |
| 761 | GCGCTTGCCCGATGCGATGCATTA | TAATGCATCGCATCGGGCAAGCGC |
| 762 | TTTCTGTAAGCGGCCTGGGGTTCA | TGAACCCCAGGCCGCTTACAGAAA |
| 763 | GGCTGAGGTGAGCGGTAAGGATGA | TCATCCTTACCGCTCACCTCAGCC |
| 764 | TCTTGGCCTCCCCGATCTAATTTG | CAAATTAGATCGGGGAGGCCAAGA |
| 765 | GGAGGTAACGCCGTGTACGTAGGA | TCCTACGTACACGGCGTTACCTCC |
| 766 | GTAATCCATTTGTGGCTGCGTCAA | TTGACGCAGCCACAAATGGATTAC |
| 767 | CAAACCCATTCCAGCAGACGCCTG | CAGGCGTCTGCTGGAATGGGTTTG |
| 768 | TAGGAGGAATTTGGCATGCGGGCG | CGCCCGCATGCCAAATTCCTCCTA |
| 769 | ATAGGTAGGATGTGCCCGGCGTTG | CAACGCCGGGCACATCCTACCTAT |
| 770 | GCAAGTGCTTAGCTCGTCAGCCTC | GAGGCTGACGAGCTAAGCACTTGC |
| 771 | CTGGCTGTGTCGCATCTCGTTAAC | GTTAACGAGATGCGACACAGCCAG |
| 772 | CTAACGTCGTCTCGCGCAATCACT | AGTGATTGCGCGAGACGACGTTAG |
| 773 | TTTTCATAAACGTTGTCCCCGAGC | GCTCGGGGACAACGTTTATGAAAA |
| 774 | AGCAGGAGGACGAACCTCCGCTCC | GGAGCGGAGGTTCGTCCTCCTGCT |
| 775 | TTCAAGCACCATCGTGCAATCCAA | TTGGATTGCACGATGGTGCTTGAA |
| 776 | AGCGTCGCCAGTGATCGCTAGTGG | CCACTAGCGATCACTGGCGACGCT |
| 777 | TACATTCCCTGCCTCCGTGGGCTT | AAGCCCACGGAGGCAGGGAATGTA |
| 778 | CGCTTCGCGTATTCAGTAGCGGTT | AACCGCTACTGAATACGCGAAGCG |
| 779 | TCGGACGCGTCGACACTCATTATA | TATAATGAGTGTCGACGCGTCCGA |
| 780 | TCTGAGCAGGCCAGCGCTCCAGCT | AGCTGGAGCGCTGGCCTGCTCAGA |
| 781 | TTGAATTGCCAAGCCCTGAAAGCC | GGCTTTCAGGGCTTGGCAATTCAA |
| 782 | AGTTTTCGCCTTGATGCGTCGGTG | CACCGACGCATCAAGGCGAAAACT |
| 783 | GTTTCATAGGCCACGCGTGCTAAA | TTTAGCACGCGTGGCCTATGAAAC |
| 16 | CATCGCTGCAAGTACCGCACTCAA | TTGAGTGCGGTACTTGCAGCGATG |

TABLE 4

| Seq. ID No. | Decoder Sequence (5′-3′) + 5′ T | Probe Sequence (5′-3′) + 5′ T |
|---|---|---|
| 17 | TTTCGCCGTCGTGTAGGCTTTTCAA | TTTGAAAAGCCTACACGACGGCGAA |
| 18 | TGTTCCCAGTGAAGCTGCGATCTGG | TCCAGATCGCAGCTTCACTGGGAAC |
| 19 | TTACTTGGCATGGAATCCCTTACGC | TGCGTAAGGGATTCCATGCCAAGTA |
| 20 | TACTAGCATATTTCAGGGCACCGGC | TGCCGGTGCCCTGAAATATGCTAGT |
| 21 | TGAACGGTCAATGAACCCGCTGTGA | TTCACAGCGGGTTCATTGACCGTTC |
| 22 | TGCGGCCTTGGTTCAATATGAATCG | TCGATTCATATTGAACCAAGGCCGC |
| 23 | TGATCGTTAGAGGGACCTTGCCCGA | TTCGGGCAAGGTCCCTCTAACGATC |
| 24 | TTGGACCTAGTCCGGCAGTGACGAA | TTTCGTCACTGCCGGACTAGGTCCA |
| 25 | TATAAACTACCCAGGACGGGCGGAA | TTTCCGCCCGTCCTGGGTAGTTTAT |
| 26 | TCATCGGTTCGCGCCAATCCAGATA | TTATCTGGATTGGCGCGAACCGATG |
| 27 | TGTCGGGCATAGAGCCGACCACCCT | TAGGGTGGTCGGCTCTATGCCCGAC |
| 28 | TCTTGGGTCATGATTCACCGTGCTA | TTAGCACGGTGAATCATGACCCAAG |
| 29 | TTGCCTAACGTGCTAATCAGCAGCG | TCGCTGCTGATTAGCACGTTAGGCA |
| 30 | TCGCATGTTGGAGCATATGCCCTGA | TTCAGGGCATATGCTCCAACATGCG |
| 31 | TAGCCACTGCATCAGTGCTGTTCAA | TTTGAACAGCACTGATGCAGTGGCT |
| 32 | TGGTTGTTTTGAGGCGTCCCACACT | TAGTGTGGGACGCCTCAAAACAACC |
| 33 | TTCGACCAAGAGCAAGGGCGGACCA | TTGGTCCGCCCTTGCTCTTGGTCGA |
| 34 | TGACATCGCTATTGCGCATGGATCA | TTGATCCATGCGCAATAGCGATGTC |
| 35 | TGAAATACGAAGTCTGCGGGAGTCG | TCGACTCCCGCAGACTTCGTATTTC |
| 36 | TTGTCATGAATGATTGATCGCGCGA | TTCGCGCGATCAATCATTCATGACA |
| 37 | TATATCGGGATTCGTTCCCGGTGAA | TTTCACCGGGAACGAATCCCGATAT |
| 38 | TGCGAGCGTACCGAAGGGCCTAGAA | TTTCTAGGCCCTTCGGTACGCTCGC |
| 39 | TTTACCGGCAGCGGACTTCCGAATT | TAATTCGGAAGTCCGCTGCCGGTAA |
| 40 | TGTAATCGAGAGCTGCGCGCCGTCT | TAGACGGCGCGCAGCTCTCGATTAC |
| 41 | TCCTGTTAGCGTAGGCGAGTCGATC | TGATCGACTCGCCTACGCTAACAGG |
| 42 | TTAGCGGACCGGCAGAATGAGTTCC | TGGAACTCATTCTGCCGGTCCGCTA |
| 43 | TGGTACATGCACTACGCGCACTCGG | TCCGAGTGCGCGTAGTGCATGTACC |
| 44 | TAATTCATCTCGGACTCCCGCGGTA | TTACCGCGGGAGTCCGAGATGAATT |
| 45 | TGCCAAATCTGGATTGGCAGGAATG | TCATTCCTGCCAATCCAGATTTGGC |
| 46 | TTGCATTTTCGGTTGAGGCACATCC | TGGATGTGCCTCAACCGAAAATGCA |
| 47 | TCCGCTCAATTCACCATGCTTCGCT | TAGCGAAGCATGGTGAATTGAGCGG |
| 48 | TCTCGGAAAGGTGCAACTTTGGTGT | TACACCAAAGTTGCACCTTTCCGAG |
| 49 | TAATTCGACCAGCAGAACGTCGCAT | TATGGGACGTTCTGCTGGTCGAATT |
| 50 | TGCCAGAGTCTCAACCTCACGGGAT | TATCCCGTGAGGTTGAGACTCTGGC |
| 51 | TCCAACAACTGGAACGGGAACCCGC | TGCGGGTTCCCGTTCCAGTTGTTGG |
| 52 | TGAGAACTGATCGCTGAGGGGCATG | TCATGCCCCTCAGCGATCAGTTCTC |
| 53 | TGGCACACTAGACTTGTGGCACCGA | TTCGGTGCCACAAGTCTAGTGTGCC |
| 54 | TTCACATCCAAATATGGTCCGCGAA | TTTCGCGGACCATATTTGGATGTGA |
| 55 | TGTCTGCCGGTGTGACCGCTTCATT | TAATGAAGCGGTCACACCGGCAGAC |
| 56 | TCATCGCAGAGCATAAACACCCTCA | TTGAGGGTGTTTATGCTCTGCGATG |
| 57 | TGTTGGTATCTATGGCAGAGGCGGA | TTCCGCCTCTGCCATAGATACCAAC |
| 58 | TACGAGGTGCCGCTGAGGTTCCATT | TAATGGAACCTCAGCGGCACCTCGT |
| 59 | TGGAATGAGTGGACCCAGGCACATT | TAATGTGCCTGGGTCCACTCATTCC |
| 60 | TTGTCAATATGCGTCCGTGTCGTCT | TAGACGACACGGACGCATATTGACA |
| 61 | TTGATGAGCCTCAGGGTACGAGGCA | TTGCCTCGTACCCTGAGGCTCATCA |
| 62 | TCACCGCGGTGTTCCTACAGAATGA | TTCATTCTGTAGGAACACCGCGGTG |
| 63 | TTTGTTGCCAATGGTGTCCGCTCGG | TCCGAGCGGACACCATTGGCAACAA |
| 64 | TTTAACCTGCGTCTGCCCCTTTCCT | TAGGAAAGGGGCAGACGCAGGTTAA |
| 65 | TAGGCGCGTTCCTGCCTTAGTGACG | TCGTCACTAAGGCAGGAACGCGCCT |
| 66 | TTAGGGCGATGGCACGAAGCTTCAA | TTTGAAGCTTCGTGCCATCGCCCTA |
| 67 | TTGCATAGAGCCAAAGTCGGCGATG | TCATCGCCGACTTTGGCTCTATGCA |
| 68 | TTTGAGAGGCAGGTGGCCACACGGA | TTCCGTGTGGCCACCTGCCTCTCAA |
| 69 | TTCCGCATTGTGAGAAAAAACGAGC | TGCTCGTTTTTTCTCACAATGCGGA |
| 70 | TGGCGGTTTCCGTAGCTATAGGTGC | TGCACCTATAGCTACGGAAACCGCC |
| 71 | TGGTGAAAATTTCGTAGCCACGGGC | TGCCCGTGGCTACGAAATTTTCACC |
| 72 | TCCGACGGAGGATGAAGACAATCAC | TGTGATTGTCTTCATCCTCCGTCGG |
| 73 | TCCAGTTTGGCCCAATTCGCCAAAA | TTTTTGGCGAATTGGGCCAAACTGG |
| 74 | TGGATCTATTAGGCCGTGCGCACAG | TCTGTGCGCACGGCCTAATAGATCC |
| 75 | TCGGATGTCACCGTTTGGACTTTCA | TTGAAAGTCCAAACGGTGACATCCG |
| 76 | TATCGCAAATCCTGCTCGTCCCTAA | TTTAGGGACGAGCAGGATTTGCGAT |
| 77 | TCAGGGCATGCAATAATCGAGGTTC | TGAACCTCGATTATTGCATGCCCTG |
| 78 | TCATGCGTTGATATATGGGCCCAAG | TCTTGGGCCCATATATCAACGCATG |
| 79 | TCAGCTGCAGCTTGTGACCAACCAC | TGTGGTTGGTCACAAGCTGCAGCTG |
| 80 | TTTGTATGTCTGCCGACCGGCGACC | TGGTCGCCGGTCGGCAGACATACAA |
| 81 | TGATGGCGCCCGTTGATAGGTATGG | TCCATACCTATCAACGGGCGCCATC |
| 82 | TATGAGAATCGCCGGCAATCTGCTA | TTAGCAGATTGCCGGCGATTCTCAT |
| 83 | TATTTGCACTGACCGCAGGCTCGTG | TCACGAGCCTGCGGTCAGTGCAAAT |
| 84 | TCAGGGAGAACGGTTAAGTTCCCGT | TACGGGAACTTAACCGTTCTCCCTG |
| 85 | TAGGCCGGCGATCGAGGAGTTTGGT | TACCAAACTCCTCGATCGCCGGCCT |
| 86 | TACACGGTGGTCTCTGATAGCGACC | TGGTCGCTATCAGAGACCACCGTGT |
| 87 | TGTGCAACGCCGAGGACTTCCATCA | TTGATGGAAGTCCTCGGCGTTGCAC |
| 88 | TTCGGTGCCTGATAGCCATTCCGAT | TATCGGAATGGCTATCAGGCACCGA |
| 89 | TTGAAATACCACACAGCCAATTGGC | TGCCAATTGGCTGTGTGGTATTTCA |
| 90 | TGCATCGTGTACATGACTGCCGCGA | TTCGCGGCAGTCATGTACACGATGC |
| 91 | TCAGTGTTCTAACGGCGCGCGTGAA | TTTCACGCGCGCCGTTAGAACACTG |
| 92 | TCGCTTGCAACGTTGCACCTACTCT | TAGAGTAGGTGCAACGTTGCAAGCG |
| 93 | TCGAAAAACTAGTGGGCTCGCCGCG | TCGCGGCGAGCCCACTAGTTTTTCG |
| 94 | TCTTTCAGGGGAACTGCCGGAGTCG | TCGACTCCGGCAGTTCCCCTGAAAG |
| 95 | TTTGTGGCCTTCTTGTAAAGGCACG | TCGTGCCTTTACAAGAAGGCCACAA |
| 96 | TTCCACGAACGGCGACCCGTTGTCT | TAGACAACGGGTCGCCGTTCGTGGA |
| 97 | TCGACCTTGCACGAAACCTAACGAG | TCTCGTTAGGTTTCGTGCAAGGTCG |
| 98 | TGTGCAGCTTCACGAGCCAGCCTGA | TTCAGGCTGGCTCGTGAAGCTGCAC |
| 99 | TCGCTTTCGTGCGAATAGACGATGA | TTCATCGTCTATTCGCACGAAAGCG |
| 100 | TTGCGCTTACAGGCTCCTAGTGGTC | TGACCACTAGGAGCCTGTAAGCGCA |
| 101 | TCACGCGCTTAGTCGCGATCGCATA | TTATGCGATCGCGACTAAGCGCGTG |
| 102 | TCGGAGGGAGGGAGCTAGCCTTCGA | TTCGAAGGCTAGCTCCCTCCCTCCG |
| 103 | TGCATCCGGCCTGTTGATGACGCCT | TAGGCGTCATCAACAGGCCGGATGC |
| 104 | TAGGCCAATCGATCTTATTGCCGAG | TCTCGGCAATAAGATCGATTGGCCT |
| 105 | TCCTTCCAATGATTGCATACGCCCA | TTGGGCGTATGCAATCATTGGAAGG |
| 106 | TAACACTTGATCAGGCGGGTCGTCT | TAGACGACCCGCCTGATCAAGTGTT |
| 107 | TTGGAATCAAGGCCGTAAAGGACAG | TCTGTCCTTTACGGCCTTGATTCCA |
| 108 | TGCTCCCGTAACCTGTCCACCAGTG | TCACTGGTGGACAGGTTACGGGAGC |
| 109 | TAGTGGTGAATGGCCGCTACCCTGA | TTCAGGGTAGCGGCCATTCACCACT |
| 110 | TTGTTGAAGCGAGCTAAAACGGCCA | TTGGCCGTTTTAGCTCGCTTCAACA |
| 111 | TCAGCGCTCCAGAATTGACAGCAAT | TATTGCTGTCAATTCTGGAGCGCTG |
| 2 | TTTCGAAGCGCACGTCCCTTTTCAA | TTTGAAAAGGGACGTGCGCTTCGAA |
| 3 | TAACGCGTGGGGAATGGGACATCAA | TTTGATGTCCCATTCCCCACGCGTT |
| 114 | TCACGAGATACCGGCGTAAGGGTGG | TCCACCCTTACGCCGGTATCTCGTG |
| 115 | TCTACGGCAAACGTGTGGAATGGGT | TACCCATTCCACACGTTTGCCGTAG |
| 116 | TGTAGGGCGATGACGGGCGAACTAC | TGTAGTTCGCCCGTCATCGCCCTAC |
| 117 | TAATCGACCTCCGCACACATTCGCA | TTGCGAATGTGTGCGGAGGTCGATT |
| 118 | TGAGTCAGCATGGCGGCGGAGATTC | TGAATCTCCGCCGCCATGCTGACTC |
| 119 | TAGATAAAGACGCTGGCAACACGGG | TCCCGTGTTGCCAGCGTCTTTATCT |
| 120 | TGGTACCTCAACGCGAACCACTTGT | TACAAGTGGTTCGCGTTGAGGTACC |
| 121 | TAAGCGATGGCTACCCAAGAGCGAT | TATCGCTCTTGGGTAGCCATCGCTT |
| 122 | TAGAGCTTATGCAGAACCAGGCGCC | TGGCGCCTGGTTCTGCATAAGCTCT |
| 123 | TATCGGTCTCACGCAGGGTTGGATA | TTATCCAACCCTGCGTGAGACCGAT |
| 124 | TTAGGTTGCCCGCCAGAAGAAACAT | TATGTTTCTTCTGGCGGGCAACCTA |
| 125 | TCGGTGCTGTTGCAAAAGCCTGTAG | TCTACAGGCTTTTGCAACAGCACCG |
| 126 | TTGATGAAAGTTTGCGGCAGGACAC | TGTGTCCTGCCGCAAACTTTCATCA |
| 127 | TGTTGAGTGCAGGATGCAGCGATAG | TCTATCGCTGCATCCTGCACTCAAG |
| 128 | TAACATTGCGCGGTCCACCAGGGTT | TAACCCTGGTGGACCGCGCAATGTT |
| 129 | TGGGCAGTTAGAGAGGGCCAGAAGT | TACTTCTGGCCCTCTCTAACTGCCC |
| 130 | TTCGAGCTGGTCCCCGTGAACGTGT | TACACGTTCACGGGGACCAGCTCGA |
| 131 | TGTCTTGGGGGCCGCTTAGTGAAAA | TTTTTCACTAAGCGGCCCCCAAGAC |
| 132 | TACTGTTGGCTTGCTCTCATGTCCA | TTGGACATGAGAGCAAGCCAACAGT |
| 133 | TAGGACCATTCGGAAGGCGAAGATA | TTATCTTCGCCTTCCGAATGGTCCT |
| 134 | TCTTGGGAGGCATCCGCTATAAGGA | TTCCTTATAGCGGATGCCTCCCAAG |
| 135 | TAATAAACGGAACGCACCGCTACAG | TCTGTAGCGGTGCGTTCCGTTTATT |
| 136 | TTTGTACGTGCGGTCCCCATAAGCA | TTGCTTATGGGGACCGCACGTACAA |
| 137 | TCGCACCAAACTGAGTTTCCCAGAC | TGTCTGGGAAACTCAGTTTGGTGCG |
| 138 | TACCTGATCGTTCCCCTATTGGGAA | TTTCCCAATAGGGGAACGATCAGGT |
| 139 | TGGAACAGAGGCGAGGGGACTGAGC | TGCTCAGTCCCCTCGCCTCTGTTCC |
| 140 | TCCCTGCCTTGGCGTGTCGGCTTAT | TATAAGCCGACACGCCAAGGCAGGG |
| 141 | TACTCTGACACGCCAACTCCGGAAG | TCTTCCGGAGTTGGCGTGTCAGAGT |
| 142 | TCTGACGGTTTTCATTCGGCGTGCC | TGGCACGCCGAATGAAAACCGTCAG |
| 143 | TTGCGGTGGTTCATTGGAGCTGGCC | TGGCCAGCTCCAATGAACCACCGCA |
| 144 | TGCATGGCCAACTAGTGACTCGCAA | TTTGCGAGTCACTAGTTGGCCATGC |
| 145 | TAGGCCGTAAAGCGAATCTCACCTG | TCAGGTGAGATTCGCTTTACGGCCT |
| 146 | TCGAATATTATGCCGAGAATCCGCG | TCGCGGATTCTCGGCATAATATTCG |
| 147 | TACAGACGAGCTCCCAACCACATGA | TTCATGTGGTTGGGAGCTCGTCTGT |
| 148 | TGGACGGTTTGTGCTGGATTGTCTG | TCAGACAATCCAGCACAAACCGTCC |
| 149 | TAAAGGCTATTGAGTTGGTTGGGCG | TCGCCCAACCAACTCAATAGCCTTT |
| 150 | TGATGGCCTATTCGGAGATCGGGCC | TGGCCCGATCTCCGAATAGGCCATC |
| 151 | TGATCCAGTAGGCAGCTTCATCCCA | TTGGGATGAAGCTGCCTACTGGATC |
| 152 | TAATAACTCGCGCGGGTATGCTTCT | TAGAAGCATACCCGCGCGAGTTATT |
| 153 | TGGAGGAGGTTTGTCTCGGAAAGCA | TTGCTTTCCGAGACAAACCTCCTCC |
| 154 | TCTTTGGTATGGCACATGCTGCCCG | TCGGGCAGCATGTGCCATACCAAAG |
| 155 | TAGAAAGGCTCGAGCAACGGGAACT | TAGTTCCCGTTGCTCGAGCCTTTCT |
| 156 | TAATCTACCGCACTGGTCCGCAAGT | TACTTGCGGACCAGTGCGGTAGATT |
| 157 | TCGTGGCGGCCACAGTTTTTGGAGG | TCCTCCAAAAACTGTGGCCGCCACG |
| 158 | TTTGCAGTTCAATCCATACGCACGT | TACGTGCGTATGGATTGAACTGCAA |
| 159 | TGGCCCAAAGCCCCAGACCATTTTA | TTAAAATGGTCTGGGGCTTTGGGCC |
| 160 | TCGCCTGTCTTTGTCTCCGGACAAT | TATTGTCCGGAGACAAAGACAGGCG |
| 161 | TTGAGGCAACAGGGGCCAAAAACTA | TTAGTTTTTGGCCCCTGTTGCCTCA |
| 162 | TAGCGGAAGTAGTCCTCGGCTCGTC | TGACGAGCCGAGGACTACTTCCGCT |
| 163 | TGGCCCCAAGGCTTAGAGATAGTGG | TCCACTATCTCTAAGCCTTGGGGCC |
| 164 | TGCACGTGAAGTTTAACCGCGATTC | TGAATCGCGGTTAAACTTCACGTGC |
| 165 | TAGCGGCAGAAACGTTCCTTGACGG | TCCGTCAAGGAACGTTTCTGCCGCT |
| 166 | TTCGTCGAGCAGACGAGATTGCACG | TCGTGCAATCTCGTCTGCTCGACGA |
| 167 | TTCTTTGCCGCGTAACTGACTGCTT | TAAGCAGTCAGTTACGCGGCAAAGA |
| 168 | TTTTATGTGCCAAGGGGTTAACCGA | TTCGGTTAACCCCTTGGCACATAAA |
| 169 | TTGTTACTGTGGTTCACGGCAGTCC | TGGACTGCCGTGAACCACAGTAACA |
| 170 | TCGCGCCTCGCTAGACCTTTTATTG | TCAATAAAAGGTCTAGCGAGGCGCG |
| 171 | TACAAATGCGTGAGAGCTCCCAACT | TAGTTGGGAGCTCTCACGCATTTGT |
| 172 | TCGCGCAGATTATAGACCCGAATGT | TACATTCGGGTCTATAATCTGCGCG |
| 173 | TCAAATAACGCCGCTGAATCGGCGT | TACGCCGATTCAGCGGCGTTATTTG |
| 174 | TCCTTCGTGCATCGGTGATGATGTT | TAACATCATCACCGATGCACGAAGG |
| 175 | TTGAACACGAGCAACACTCCAACGC | TGCGTTGGAGTGTTGCTCGTGTTCA |
| 176 | TCAGCAGATCCTTCGTAGCGGTCGT | TACGACCGCTACGAAGGATCTGCTG |
| 177 | TGGAACCTGGTGAGTTGTGCCTCAT | TATGAGGCACAACTCACCAGGTTCC |
| 178 | TTCATAAGCGACAATCGCGGGCTTA | TTAAGCCCGCGATTGTCGCTTATGA |
| 179 | TCCCAACGTCACTGAAGCTCACAGT | TACTGTGAGCTTCAGTGACGTTGGG |
| 180 | TTGTCAGAGCCCGCGACTCAGACGG | TCCGTCTGAGTCGCGGGCTCTGACA |
| 181 | TTACACGAAGCCTCTCCGTGGTCCA | TTGGACCACGGAGAGGCTTCGTGTA |
| 182 | TCTCAGAAGTCCTCGGCGAACTGGG | TCCCAGTTCGCCGAGGACTTCTGAG |
| 183 | TATCCTTTTATCTACTCCGCGGCGA | TTCGCCGCGGAGTAGATAAAAGGAT |
| 184 | TAGGCGTGCAGCAACAGGATAAACC | TGGTTTATCCTGTTGCTGCACGCCT |
| 185 | TACTCTCGAGGGAGTCTCTGGCACA | TTGTGCCAGAGACTCCCTCGAGAGT |
| 186 | TTTGCCAGGTCCATCGAGACCTGTT | TAACAGGTCTCGATGGACCTGGCAA |
| 187 | TTCCACTATAACTGCGGGTCCGTGT | TACACGGACCCGCAGTTATAGTGGA |
| 188 | TGCCCAGTCGGCTCTAACAAGTTCG | TCGAACTTGTTAGAGCCGACTGGGC |
| 189 | TCGGAACGGATAATCGGCGTCAGGT | TACCTGACGCCGATTATCCGTTCCG |
| 190 | TTAAAATAAGCGCCTGGCGGGAGGA | TTCCTCCCGCCAGGCGCTTATTTTA |
| 191 | TGCGCACTCGTGAAACCTTTCTCGC | TGCGAGAAAGGTTTCACGAGTGCGC |
| 192 | TAGTTTGCCAGGTACTGGCAAGTGC | TGCACTTGCCAGTACCTGGCAAACT |
| 193 | TACAACGAGGGATGTCCAGCGGCAT | TATGCCGCTGGACATCCCTCGTTGT |
| 194 | TTTCGCAGCACCCGCTAGGTACAGT | TACTGTACCTAGCGGGTGCTGCGAA |
| 195 | TTAACCCGATTTTTGCGACTCTGCC | TGGCAGAGTCGCAAAAATCGGGTTA |
| 196 | TCGTCGCATTGCAAGCGTAGGCTTG | TCAAGCCTACGCTTGCAATGCGACG |
| 197 | TGAGCTGACGTCACCATCAGAGGAA | TTTCCTCTGATGGTGACGTCAGCTC |
| 198 | TGGAGGCTGGGGGTCGCGCTTAAGT | TACTTAAGCGCGACCCCCAGCCTCC |
| 199 | TTTGTGGGAACCGCACTAGCTGGCT | TAGCCAGCTAGTGCGGTTCCCACAA |
| 200 | TCCCTCGCACTGTGTTCACGCTCTT | TAAGAGGGTGAACACAGTGCGAGGG |
| 201 | TTCATTGACTCGAATCCGCACAACG | TCGTTGTGCGGATTCGAGTCAATGA |
| 202 | TACAGGGGTTGGCCTTCGTACGTAC | TGTACGTACGAAGGCCAACCCCTGT |
| 203 | TAGGCCGTGCAACATCACACAGGAT | TATCCTGTGTGATGTTGCACGGCCT |
| 204 | TGGGCCGTGGTCACGTAATATTGGC | TGCCAATATTACGTGACCACGGCCC |
| 205 | TGCGCGGACATGAAACGACAAGGCC | TGGCCTTGTCGTTTCATGTCCGCGC |
| 206 | TCTTATTGGGTGCCGGTGTCGGATT | TAATCCGACACCGGCACCCAATAAG |
| 207 | TGGGGCGGTTACCAAAAAATCCGAT | TATCGGATTTTTTGGTAACCGCCCC |
| 4 | TCCGTCGCATACCGGCTACGATCAA | TTTGATCGTAGCCGGTATGCGACGG |
| 5 | TATGGCCGTGCTGGGGACAAGTCAA | TTTGACTTGTCCCCAGCACGGCCAT |
| 210 | TACGAAAAAAGTGTGCGGATCCCCT | TAGGGGATCCGCACACTTTTTTCGT |
| 211 | TCCAAGTACACCGCACGCATGTTTA | TTAAACATGCGTGCGGTGTACTTGG |
| 212 | TATCGTGCGTGGAGTGTCGCATCTA | TTAGATGCGACACTCCACGCACGAT |
| 213 | TTCCAGATACCGCCCCGAACTTTGA | TTCAAAGTTCGGGGCGGTATCTGGA |
| 214 | TTCTGCTGGCAGCACGTGAAGTGGC | TGCCACTTCACGTGCTGCCAGCAGA |
| 215 | TTTGAAATTGCTCTGCCGTCAGTCA | TTGACTGACGGCAGAGCAATTTCAA |
| 216 | TAGTCAGGCGAGATGTTCAGGCAGC | TGCTGCCTGAACATCTCGCCTGACT |
| 217 | TACAAGCCGACGTTAAGCCCGCCCA | TTGGGCGGGCTTAACGTCGGCTTGT |
| 218 | TCCCTAATGAGGCCAGTAACCTGCA | TTGCAGGTTACTGGCCTCATTAGGG |
| 219 | TGTGAGACACACATCCCCTCCAATG | TCATTGGAGGGGATGTGTGTCTCAC |
| 220 | TCGACGGATGCAGAGTTCAGTGGTC | TGACCACTGAACTCTGCATCCGTCG |
| 221 | TCCCGCATGCCTGGCGGTATTACAA | TTTGTAATACCGCCAGGCATGCGGG |
| 222 | TTTAGCAAAGCGGCGCCGTTAGCAA | TTTGCTAACGGCGCCGCTTTGCTAA |
| 223 | TCCCGACACGGGTCAGCGTAATAAT | TATTATTACGCTGACCCGTGTCGGG |
| 224 | TGCGACGGCCCTGAGGTATGTCGTC | TGACGACATACCTCAGGGCCGTCGC |
| 225 | TCAAAAGTGTGTTCCCTTGCGCTTG | TCAAGCGCAAGGGAACACACTTTTG |
| 226 | TTCTCGAAGCACAGCCCGGTTATTG | TCAATAACCGGGCTGTGCTTCGAGA |
| 227 | TATGCTAACCGTTGGCCATGGAACT | TAGTTCCATGGCCAACGGTTAGCAT |
| 228 | TCTTGCGGAGTGTTAGCCCAGCGGT | TACCGCTGGGCTAACACTCCGCAAG |
| 229 | TTGCTCCCTAGGCGCTCGGAGGAGT | TACTCCTCCGAGCGCCTAGGGAGCA |
| 230 | TCCAATGCCTTTGAGTAAGCGATGG | TCCATCGCTTACTCAAAGGCATTGG |
| 231 | TAGCAGATAACGTCCCAATGACGCC | TGGCGTCATTGGGACGTTATCTGCT |
| 232 | TTTGACCATTACGTGTTGCGCCCAT | TATGGGCGCAACACGTAATGGTCAA |
| 233 | TTCGCGTATTTGCGGAATTCGTCTG | TCAGACGAATTCCGCAAATACGCGA |
| 234 | TCTGCGTGTCAACAATGTCCCGCAG | TCTGCGGGACATTGTTGACACGCAG |
| 235 | TTCTGGTGCCACGCAAGGTCCACAG | TCTGTGGACCTTGCGTGGCACCAGA |
| 236 | TCTCCGGGAGGTCACTTAATTGCGG | TCCGCAATTAAGTGACCTCCCGGAG |
| 237 | TTTTTCGTGATTGCCCGGAGGAGGC | TGCCTCCTCCGGGCAATCACGAAAA |
| 238 | TTCGGGATGTAGCTGGGGCTACCGG | TCCGGTAGCCCCAGCTACATCCCGA |
| 239 | TCGAGCCAACGCAAACACGTCCTTG | TCAAGGACGTGTTTGCGTTGGCTCG |
| 240 | TGCAAAGCCTTTGTGGGGCGGTAGT | TACTACCGCCCCACAAAGGCTTTGC |
| 241 | TATTCGACCGGAAATGAGGTCTTCG | TCGAAGACCTCATTTCCGGTCGAAT |
| 242 | TTTCGCTTGCTGAGTTGCTCTGTTC | TGAACAGAGCAACTCAGCAAGCGAA |
| 243 | TCGCGTGAAGACCCCATTCCCGAGT | TACTCGGGAATGGGGTCTTCACGCG |
| 244 | TAACCGTATTCGCGGTCACTTGTGG | TCCACAAGTGACCGCGAATACGGTT |
| 245 | TGGGGCCAACCGTTTCGAGGCGTAT | TATACGCCTCGAAACGGTTGGCCCC |
| 246 | TTTCGGCTGGCAGTCCAAACGGCTT | TAAGCCGTTTGGACTGCCAGCCGAA |
| 247 | TGGGTGTGGTTAGAATGCACGGTTC | TGAACCGTGCATTCTAACCACACCC |
| 248 | TGCGAGGACCGAACTAGACAAACGG | TCCGTTTGTCTAGTTCGGTCCTCGC |
| 249 | TACGCACGCGTGACCGAAGTTGCTG | TCAGCAACTTCGGTCACGCGTGCGT |
| 250 | TTAAAAGGTCGCTTTGAAAGGGGGA | TTCCCCCTTTCAAAGCGACCTTTTA |
| 251 | TTGCGATCGCTAACTGCTGGGACAA | TTTGTCCCAGCAGTTAGCGATCGCA |
| 252 | TGGAGGTATAAGCGGAGCGGCCTCA | TTGAGGCCGCTCCGCTTATACCTCC |
| 253 | TATGCTGACATGTCGTGCACCTCGT | TACGAGGTGCACGACATGTCAGCAT |
| 254 | TTGTGGTTAAAGCGTCCGTTCAACG | TCGTTGAACGGACGCTTTAACCACA |
| 255 | TCGTTCACACCGGCGTAAGCTGCGT | TACGCAGCTTACGCCGGTGTGAACG |
| 256 | TCCTATCCCGGCGAGAACTTCTGTG | TCACAGAAGTTCTCGCCGGGATAGG |
| 257 | TGTCTGCACTCACGCAGCGGAGGGA | TTCCCTCCGCTGCGTGAGTGCAGAC |
| 258 | TGCACGAGTTGGTGCTCGGCAGATT | TAATCTGCCGAGCACCAACTCGTGC |
| 259 | TAACGTCGCACGACACACGTTCGTC | TGACGAACGTGTGTCGTGCGACGTT |
| 260 | TATGCGCGCTTATCCTAGCATGGTC | TGACCATGCTAGGATAAGCGCGCAT |
| 261 | TTCACGTTTTCGTCTCGACATGAGG | TCCTCATGTCGAGACGAAAACGTGA |
| 262 | TTGTGCCTCATCCTTAGGATACGGC | TGCCGTATCCTAAGGATGAGGCACA |
| 263 | TAGGTGGTGTGGGTCAACCGCTTTA | TTAAAGCGGTTGACCCACACCACCT |
| 264 | TCTGGATCGAAGGGACTGCAAGCTC | TGAGCTTGCAGTCCCTTCGATCCAG |
| 265 | TTAGATCAACTCGCGTACGCATGGA | TTCCATGCGTACGCGAGTTGATCTA |
| 266 | TGATCCTGCGGAGAAGAGAGTGCAG | TCTGCACTCTCTTCTCCGCAGGATC |
| 267 | TTACGTGTGGAGATGCCCCGAACCG | TCGGTTCGGGGCATCTCCACACGTA |
| 268 | TGCGCTATGTCAATCGTGGGCGTAG | TCTACGCCCACGATTGACATAGCGC |
| 269 | TAGCGAGGTTTCTAGCGTCGACACC | TGGTGTCGACGCTAGAAACCTCGCT |
| 270 | TACCCAGGTTTTGCCGTTGTGGAAT | TATTCCACAACGGCAAAACCTGGGT |
| 271 | TCCCTGTTAACGGCTGCGTAGTCTC | TGAGACTACGCAGCCGTTAACAGGG |
| 272 | TAGGCCGATTTCACCCGCCAATTGC | TGCAATTGGCGGGTGAAATCGGCCT |
| 273 | TGAGCCCTCACTCCTTGCCCTTTGA | TTCAAAGGGCAAGGAGTGAGGGCTC |
| 274 | TGGGTGGACATCCGCCTCGCAGTCA | TTGACTGCGAGGCGGATGTCCACCC |
| 275 | TGATGGCTGAGAACCGTGCTACGAT | TATCGTAGCACGGTTCTCAGCCATC |
| 276 | TTCGACGTTAGGAGTGCTGCCAGAA | TTTCTGGCAGCACTCCTAACGTCGA |
| 277 | TCGAATGGGTCTGGACCTTGCATAG | TCTATGCAAGGTCCAGACCCATTCG |
| 278 | TGTGCACCAGACATTCGAACTCGGA | TTCCGAGTTCGAATGTCTGGTGCAC |
| 279 | TAGAGGCCCCGTATATCCCATCCAT | TATGGATGGGATATACGGGGCCTCT |
| 280 | TAACGCCTGTTCAGAGCATCAGCGG | TCCGCTGATGCTCTGAACAGGCGTT |
| 281 | TAAGGCTCAACACGCCTATGTGCGC | TGCGCACATAGGCGTGTTGAGCCTT |
| 282 | TAGTCCGTGTTGCCAGATTGGCTCG | TCGAGCCAATCTGGCAACACGGACT |
| 283 | TATGTCCCATGTAAAGACGCGTGTG | TCACACGCGTCTTTACATGGGACAT |
| 284 | TATGGAGTCTGCTCACGCCCAAAGG | TCCTTTGGGCGTGAGCAGACTCCAT |
| 285 | TCGGCCTCCAACAAGGAGCACTAAC | TGTTAGTGCTCCTTGTTGGAGGCCG |
| 286 | TCAGAGCCGTGGCAACATTGCGAGC | TGCTCGCAATGTTGCCACGGCTCTG |
| 287 | TTCATTTGAATGAGGTGCGCACCGG | TCCGGTGCGCACCTCATTCAAATGA |
| 288 | TGACGTACCGGAAGCGCCGTATAAA | TTTTATACGGCGCTTCCGGTACGTC |
| 289 | TATGCGAGCAATGGGATCCGGATTC | TGAATCCGGATCCCATTGCTCGCAT |
| 290 | TAGAGTGAGGCCTCCCTGACCAGTG | TCACTGGTCAGGGAGGCCTCACTCT |
| 291 | TCGCACCGTAAGTAGATTTGCCCGC | TGCGGGCAAATCTACTTACGGTGCG |
| 292 | TTGAACCTTTGAGCACGTCGTGCGC | TGCGCACGACGTGCTCAAAGGTTCA |
| 293 | TTCCGCCTTTTTGGTTACCTCGAAG | TCTTCGAGGTAACCAAAAAGGCGGA |
| 294 | TGAACGCCAACGGCACTAACACATC | TGATGTGTTAGTGCCGTTGGCGTTC |
| 295 | TCCGACAGCAGCCAAGACGTCCCAG | TCTGGGACGTCTTGGCTGCTGTCGG |
| 296 | TCATAAAAAAACCTGGGGCTCTGCG | TCGCAGAGCCCCAGGTTTTTTTATG |
| 297 | TTGCCAACTGTGCAGACCGGACTTA | TTAAGTCCGGTCTGCACAGTTGGCA |
| 298 | TGGCGAAAGAGCGAAACCGGCTCGT | TACGAGCCGGTTTCGCTCTTTCGCC |
| 299 | TGGGATGCGTATTTTAGCGAACACG | TCGTGTTCGCTAAAATACGCATCCC |
| 300 | TTGGGATTCAGCGACCAGTACGCGA | TTCGCGTACTGGTCGCTGAATCCCA |
| 301 | TCCCGATATTCGCCCGGCCTATTCG | TCGAATAGGCCGGGCGAATATCGGG |
| 302 | TCGAGAAGATGCCTCACGCAACCAA | TTTGGTTGCGTGAGGCATCTTCTCG |
| 303 | TAACCTTGACCCGTGGATGACGCTA | TTAGCGTCATCCACGGGTCAAGGTT |
| 6 | TTTGCAACGGGCTGGTCAACGTCAA | TTTGACGTTGACCAGCCCGTTGCAA |
| 7 | TCGCATAGGTTGCCGATTTCGTCAA | TTTGACGAAATCGGCAACCTATGCG |
| 306 | TGCTTCCGGATGAACGGGATGGTTG | TCAACCATCCCGTTCATCCGGAAGC |
| 307 | TCCCTCCATGTTCTTCGAACGGTTT | TAAACCGTTCGAAGAACATGGAGGG |
| 308 | TTTGATGGGCGGCAATGCTCTTGCT | TAGCAAGAGCATTGCCGCCCATCAA |
| 309 | TATTGTGAGATGCGCCAAATTCCCC | TGGGGAATTTGGCGCATCTCACAAT |
| 310 | TTCAGCACAGCCAGACGGTCAACTT | TAAGTTGACCGTCTGGCTGTGCTGA |
| 311 | TACTCCACTCCTCGGTGGCAAACTA | TTAGTTTGCCACCGAGGAGTGGAGT |
| 312 | TTCTGGGCATGCCTGGACGGAGACG | TCGTCTCCGTCCAGGCATGCCCAGA |
| 313 | TTCTCAACTCCGGTACGACGAAACA | TTGTTTCGTCGTACCGGAGTTGAGA |
| 314 | TTTGCGTGGTCAAAGGCGCAACGTG | TCACGTTGCGCCTTTGACCACGCAA |
| 315 | TAGACAGCGATCCGCGGCTCATGAT | TATCATGAGCCGCGGATCGCTGTCT |
| 316 | TCGCGTCTCTAACTGAGAGCAGCCA | TTGGCTGCTCTCAGTTAGAGACGCG |
| 317 | TAGGCGCACATGTACGGACATTCAG | TCTGAATGTCCGTACATGTGCGCCT |
| 318 | TGATGAGTGGCACGTCGGTGTGTAA | TTTACACACCGACGTGCCACTCATC |
| 319 | TTGATCCATATTGTCGGACGTTGCG | TCGCAACGTCCGACAATATGGATCA |
| 320 | TACCTGCCGGGAGTTCATAGGCTAG | TCTAGCCTATGAACTCCCGGCAGGT |
| 321 | TAGCATTGGCGTTTTTCCGCAACGA | TTCGTTGCGGAAAAACGCCAATGCT |
| 322 | TGGTAATATTCAGCGCGACCGCTCA | TTGAGCGGTCGCGCTGAATATTACC |
| 323 | TATAGCGTACGACGAGGTGACGCGC | TGCGCGTCACCTCGTCGTACGCTAT |
| 324 | TTAGGTCACGATGCGTTTGACGCTA | TTAGCGTCAAACGCATCGTGACCTA |
| 325 | TACTGCCCGTACCTCTGGTTCTGGC | TGCCAGAACCAGAGGTACGGGCAGT |
| 326 | TCCTTTGGCCTGAAGTTGTCGTAGC | TGCTACGACAACTTCAGGCCAAAGG |
| 327 | TGTGCCCCACGAGCGTATCGTTGTA | TTACAACGATACGCTCGTGGGGCAC |
| 328 | TAGGCGCTACGTGGGCCTGGAGCAA | TTTGCTCCAGGCCCACGTAGCGCCT |
| 329 | TGGGTGCTACCATTGCATTAGTCCG | TCGGACTAATGCAATGGTAGCACCC |
| 330 | TACCACGCGCGTACGTGTAACCGAG | TCTCGGTTACACGTACGCGCGTGGT |
| 331 | TCCATGATGCATTGGGTGCATTTAG | TCTAAATGCACCCAATGCATCATGG |
| 332 | TGGTCCGGCCCTACGAAACGTTCGA | TTCGAACGTTTCGTAGGGCCGGACC |
| 333 | TCCGTGTGGCTGGAGATTCGTGTGA | TTCACACGAATCTCCAGCCACACGG |
| 334 | TGTTAGGGCGACGCATATTGGCACA | TTGTGCCAATATGCGTCGCCCTAAC |
| 335 | TGGGTCAGTCAGGTGCGTTAGGATC | TGATCCTAACGCACCTGACTGACCC |
| 336 | TGCCGTGAAGTCGAATGCAGATCGA | TTCGATCTGCATTCGACTTCACGGC |
| 337 | TGCCACCACCCAGTGCATTCAGGTA | TTACCTGAATGCACTGGGTGGTGGC |
| 338 | TGAGCTTAGTTTGCGGTCATCGGGC | TGCCCGATGACCGCAAACTAAGCTC |
| 339 | TTGTTTGCCGCCATTAGGGAGTAAC | TGTTACTCCCTAATGGCGGCAAACA |
| 340 | TGCTCCGCTGGATGTGCCGGTTTAG | TCTAAACCGGCACATCCAGCGGAGC |
| 341 | TCGGTAGCATGCGAGATCCCTGTTA | TTAACAGGGATCTCGCATGCTACCG |
| 342 | TCTACGCTCTACCAGTTGCCTGCGA | TTCGCAGGCAACTGGTAGAGCGTAG |
| 343 | TGTGCCTCCTGCTGTATTTGCCAAG | TCTTGGCAAATACAGCAGGAGGCAC |
| 344 | TTTGCGACTCGACTTGGACGAGTAG | TCTACTCGTCCAAGTCGAGTCGCAA |
| 345 | TTCTGGGAGCTGTTTACTCCAGCCA | TTGGCTGGAGTAAACAGCTCCCAGA |
| 346 | TTGCACGCGGAACTCCCTTTACCAT | TATGGTAAAGGGAGTTCCGCGTGCA |
| 347 | TTGGCAGCAAATGAATCGAAAGCAC | TGTGCTTTCGATTCATTTGCTGCCA |
| 348 | TAACTGGTGACGCGGTACAGCGAAG | TCTTCGCTGTACCGCGTCACCAGTT |
| 349 | TAGACGATTACGCTGGACGCCGTCG | TCGACGGCGTCCAGCGTAATCGTCT |
| 350 | TATGCCCTCCTTCATGGAAAGGGTT | TAACCCTTTCCATGAAGGAGGGCAT |
| 351 | TATTCTCGGAGCGTATGCGCCAGAA | TTTCTGGCGCATACGCTCCGAGAAT |
| 352 | TATAGCGGAGTTTGGGTACGCGAAC | TGTTCGCGTACCCAAACTCCGCTAT |
| 353 | TACCTACGCATACCGCTTGGCGAGG | TCCTCGCCAAGCGGTATGCGTAGGT |
| 354 | TGATTACCTGAATGGCCAAGCGAGC | TGCTCGCTTGGCCATTCAGGTAATC |
| 355 | TCCTGTTAGCATCACGGCGCTTAGG | TCCTAAGCGCCGTGATGCTAACAGG |
| 356 | TCGGAATGATGCGCTCGACAACGCT | TAGCGTTGTCGAGCGCATCATTCCG |
| 357 | TTGAGAGAGGCGTTGGTTAAGGCAA | TTTGCCTTAACCAACGCCTCTCTCA |
| 358 | TAAGCAGGCGAAGGGATACTCCTCG | TCGAGGAGTATCCCTTCGCCTGCTT |
| 359 | TTCACGACAGACGGGCCGAGATTAC | TGTAATCTCGGCCCGTCTGTCGTGA |
| 360 | TAAGCAATTTGGCCTCGTTTTGTGA | TTCACAAAACGAGGCCAAATTGCTT |
| 361 | TGCTGGTTGCGGTAGGATCGCATAT | TATATGCGATCCTACCGCAACCAGC |
| 362 | TTTGTGAATCCGTTCTGTCCCCGAC | TGTCGGGGACAGAACGGATTCACAA |
| 363 | TTGGGCTCCTCTGAGGCGAGATGGC | TGCCATCTCGCCTCAGAGGAGCCCA |
| 364 | TGGATAGAGTGAATCGACCGGCAAC | TGTTGCCGGTCGATTCACTCTATCC |
| 365 | TTGCACCGAACGTGCACGAGTAATT | TAATTACTCGTGCACGTTCGGTGCA |
| 366 | TGCCAGTATTCTCGGGTGTTGGACG | TCGTCCAACACCCGAGAATACTGGC |
| 367 | TTCGCTACCTAAGACCGGGCCATAC | TGTATGGCCCGGTCTTAGGTAGCGA |
| 368 | TTGGCATTGACGAGCAGCAGTCAGT | TACTGACTGCTGCTCGTCAATGCCA |
| 369 | TCGCGTCCCAGCGCCCTTGGAGTAT | TATACTCCAAGGGCGCTGGGACGCG |
| 370 | TATGAAGCCTACCGGGCGACTTCGT | TACGAAGTCGCCCGGTAGGCTTCAT |
| 371 | TCCAGACAGATGGCCTGGAACCATG | TCATGGTTCCAGGCCATCTGTCTGG |
| 372 | TTGGCGTGGGACCATCTCAAAGCTA | TTAGCTTTGAGATGGTCCCACGCCA |
| 373 | TCCGCATGGGAACACGTGTCAAGGT | TACCTTGACACGTGTTCCCATGCGG |
| 374 | TGCCCACTCGTCAGCTGGACGTAAT | TATTACGTCCAGCTGACGAGTGGGC |
| 375 | TATTACGGTCGTGATCCAGAAAGCG | TCGCTTTCTGGATCACGACCGTAAT |
| 376 | TTGCGAGGTGAGCACCTACGAGAGA | TTCTCTCGTAGGTGCTCACCTCGCA |
| 377 | TGGGCCGCATTCTTGATGTCCATTC | TGAATGGACATCAAGAATGCGGCCC |
| 378 | TCCTCGGATGTGGGCTCTCGCCTAG | TCTAGGCGAGAGCCCACATCCGAGG |
| 379 | TTAGGCATGTTGGCGTGAGCGCTAT | TATAGCGCTCACGCCAACATGGCTA |
| 380 | TCGATACGAACGAGGATGTCCGCCT | TAGGCGGACATCCTCGTTCGTATCG |
| 381 | TTACGCCGGTTAGCACGGTGCGCTA | TTAGCGCACCGTGCTAACCGGCGTA |
| 382 | TCATACGATGTCCGGGCCGTGTCGC | TGCGACACGGCCCGGACATCGTATG |
| 383 | TATCCGCAGTTGTATGGCGCGTTAT | TATAACGCGCCATACAACTGCGGAT |
| 384 | TGGGTAAGGGACAAAGATGGGATGG | TCCATCCCATCTTTGTCCCTTACCC |
| 385 | TATTGGAGTGTTTTGGTGAATCCGC | TGCGGATTCACCAAAACACTCCAAT |
| 386 | TGAACCGAGCCAACGTATGGACACG | TCGTGTCCATACGTTGGCTCGGTTC |
| 387 | TGCCGTCAAGCTTAAGGTTTTGGGC | TGCCCAAAACCTTAAGCTTGACGGC |
| 388 | TACCTGCTTTTGGGTGGGTGATATG | TCATATCACCCACCCAAAAGCAGGT |
| 389 | TAATCGTGGGCGCAGCAAACGTATA | TTATACGTTTGCTGCGCCCACGATT |
| 390 | TGTCGCCGGATTGCTCAGTATAAGC | TGCTTATACTGAGCAATCCGGCGAC |
| 391 | TACCCGTCGATGCTTCCTCCTCAGA | TTCTGAGGAGGAAGCATCGACGGGT |
| 392 | TATCCGGGTGGGCGATACAAGAGAT | TATCTCTTGTATCGCCCACCCGGAT |
| 393 | TTTCCGCATGAGTCAGCTTTGAAAA | TTTTTCAAAGCTGACTCATGCGGAA |
| 394 | TGCAAAGTCCCACTGGCAAGCCGAT | TATCGGCTTGCCAGTGGGACTTTGC |
| 395 | TCGACCTCGGCTTCATCGTACACAT | TATGTGTACGATGAAGCCGAGGTCG |
| 396 | TCTCATGAGCGCAGTTGTGCGTGAG | TCTCACGCACAACTGCGCTCATGAG |
| 397 | TCAGATGAAGGATCCACGGCCGGAG | TCTCCGGCCGTGGATCCTTCATCTG |
| 398 | TTCAAAGGCTCTTGGATACAGCCGT | TACGGCTGTATCCAAGAGCCTTTGA |
| 399 | TTCCGCTAATTTCCAATCAGGGCTC | TGAGCCCTGATTGGAAATTAGCGGA |
| 8 | TCCGTTTGCGGTCGTCCTTGCTCAA | TTTGAGCAAGGACGACCGCAAACGG |
| 9 | TTTCGCTTTCGTGGCTGCACTTCAA | TTTGAAGTGCAGCCACGAAAGCGAA |
| 402 | TCTTAGTTGGGGCGCGGTATCCAGA | TTCTGGATACCGCGCCCCAACTAAG |
| 403 | TGCTCTAATGCCGTGGAGTCGGAAC | TGTTCCGACTCCACGGCATTAGAGC |
| 404 | TCCGATTACAAATTGACTGACCGCA | TTGCGGTCAGTCAATTTGTAATCGG |
| 405 | TAGACGTACGTGAGCCTCCCGTGTC | TGACACGGGAGGCTCACGTACGTCT |
| 406 | TAATGGAGCGATACGATCCAACGCA | TTGCGTTGGATCGTATCGCTCCATT |
| 407 | TGGAGGCGCTGTACTGATAGGCGTA | TTACGCCTATCAGTACAGCGCCTCC |
| 408 | TTGTTTTTGAATTGACCACACGGGA | TTCCCGTGTGGTCAATTCAAAAACA |
| 409 | TCATGTCTGGATGCGCTCAATGAAG | TCTTCATTGAGCGCATCCAGACATG |
| 410 | TGCCCGCTAATCCGACACCCAGTTT | TAAACTGGGTGTCGGATTAGCGGGC |
| 411 | TCCATTGACAGGAGAGCCATGAGCC | TGGCTCATGGCTCTCCTGTCAATGG |
| 412 | TGAATCACCGAATCACCGACTCGTT | TAACGAGTCGGTGATTCGGTGATTC |
| 413 | TAACCAGCCGCAGTAGCTTACGTCG | TCGACGTAAGCTACTGCGGCTGGTT |
| 414 | TTTTTCTGAGGGACACGCGGGCGTT | TAACGCCCGCGTGTCCCTCAGAAAA |
| 415 | TGGTGCTCCGTTTGATCGATCCTCC | TGGAGGATCGATCAAACGGAGCACC |
| 416 | TCCGCTTAGGCCATACTCTGAGCCA | TTGGCTCAGAGTATGGCCTAAGCGG |
| 417 | TTAAGACATACCGACGCCCTTGCCT | TAGGCAAGGGCGTCGGTATGTCTTA |
| 418 | TGTTCCCGACGCCAGTCATTGAGAC | TGTCTCAATGACTGGCGTCGGGAAC |
| 419 | TTAAAAGTTTCGCGGAGGTCGGGCT | TAGCCCGACCTCCGCGAAACTTTTA |
| 420 | TCGGTCCAGACGAGCTGAGTTCGGC | TGCCGAACTCAGCTCGTCTGGACCG |
| 421 | TCGGCGTAGCGGCTACGGACTTAAA | TTTTAAGTCCGTAGCCGCTACGCCG |
| 422 | TGCTTGGATGCCCATGCGGCAAGGT | TACCTTGCCGCATGGGCATCCAAGC |
| 423 | TAGCGGGATCCCAGAGTTTCGAAAA | TTTTTCGAAACTCTGGGATCCCGCT |
| 424 | TGAGCTTGAGAGCGAGGTCATCCTC | TGAGGATGACCTCGCTCTCAAGCTC |
| 425 | TGCATCGGCCGTTTTGACCATATTC | TGAATATGGTCAAAACGGCCGATGC |
| 426 | TCATAGCGCTGCACGTTTCGACCGC | TGCGGTCGAAACGTGCAGCGCTATG |
| 427 | TACCCGACAACCACCAATTCAAAAA | TTTTTTGAATTGGTGGTTGTCGGGT |
| 428 | TGCGAACACTCATAAGAGCGCCCTG | TCAGGGCGCTCTTATGAGTGTTCGC |
| 429 | TCCGCCGAGTGTAGAGAGACTCCGA | TTCGGAGTCTCTCTACACTCGGCGG |
| 430 | TGACATCGGGAGCCGGAAACATGAG | TCTCATGTTTCCGGCTCCCGATGTC |
| 431 | TTCGTGTAGACTCGGCGACAGGCGT | TACGCCTGTCGCCGAGTCTACACGA |
| 432 | TATGCGCATATACTGACTGCGCAGG | TCCTGCGCAGTCAGTATATGCGCAT |
| 433 | TACAAGCGAACCCGAGTTTTGATGA | TTCATCAAAACTCGGGTTCGCTTGT |
| 434 | TGCATGAGACTCCGCGAAGACATGT | TACATGTCTTCGCGGAGTCTCATGC |
| 435 | TTCCTACATGTCGCGTCACGATCAC | TGTGATCGTGACGCGACATGTAGGA |
| 436 | TGACCGATCGCGAAGTCGTACACAT | TATGTGTACGACTTCGCGATCGGTC |
| 437 | TGTCGCCAGGACTGGGCCGATGTGA | TTCACATCGGCCCAGTCCTGGCGAC |
| 438 | TACCGATAAGACTTGCATCCGAACG | TCGTTCGGATGCAAGTCTTATCGGT |
| 439 | TTCCATAACCAGTCCGAAGTGCCGG | TCCGGCACTTCGGACTGGTTATGGA |
| 440 | TACGCGCCCTGCATCTCGTATTTAA | TTTAAATACGAGATGCAGGGCGCGT |
| 441 | TAGACCGCATCAATTGGCGCGTACC | TGGTACGCGCCAATTGATGCGGTCT |
| 442 | TAGAGGCTTGGCAAGTAGGGACCCT | TAGGGTCCCTACTTGCCAAGCCTCT |
| 443 | TGCAATGGACGCCAGACGATACCGG | TCCGGTATCGTCTGGCGTCCATTGC |
| 444 | TGCTGGACTTAGTCGTGTTCGGCGG | TCCGCCGAACACGACTAAGTCCAGC |
| 445 | TAGGCATCGTGCCGGATTGCTCCCT | TAGGGAGCAATCCGGCACGATGCCT |
| 446 | TTGCGCATGTCGACGTTGAACAAAG | TCTTTGTTCAACGTCGACATGCGCA |
| 447 | TTTCGGGTCACATCCGATGCCATAC | TGTATGGCATCGGATGTGACCCGAA |
| 448 | TACCCATCGCCGGAAAGCGATGTTG | TCAACATCGCTTTCCGGCGATGGGT |
| 449 | TAAGCGCTGACTCGGCTAAGAATCA | TTGATTCTTAGCCGAGTCAGCGCTT |
| 450 | TACTTCCAAGTCCTTGACCGTCCGA | TTCGGACGGTCAAGGACTTGGAAGT |
| 451 | TTCTCAATATTCCCGTAGTCGCCCA | TTGGGCGACTACGGGAATATTGAGA |
| 452 | TAACAGTTCCTCTTTTTCCTGGCGC | TGCGCCAGGAAAAAGAGGAACTGTT |
| 453 | TCGTCCTCCATGTTGTCACGAACAG | TCTGTTCGTGACAACATGGAGGACG |
| 454 | TTGCGCAGACCTACCTGTCTTTGCT | TAGCAAAGACAGGTAGGTCTGCGCA |
| 455 | TATGGACGGCTTCGCAGTCCTCCTT | TAAGGAGGACTGCGAAGCCGTCCAT |
| 456 | TTGAACGCTTTCTATGGGCCACGTA | TTACGTGGCCCATAGAAAGCGTTCA |
| 457 | TTGAACCCTGCCGCGAGCGATAACC | TGGTTATCGCTCGCGGCAGGGTTCA |
| 458 | TGTTCTTGCGCGATGAATCAGGACC | TGGTCCTGATTCATCGCGCAAGAAC |
| 459 | TAGGGTACGTGTCGCAGCTTCGCGT | TACGCGAAGCTGCGACACGTACCCT |
| 460 | TACCCTTGCTCCGCCATGTCTCTCA | TTGAGAGACATGGCGGAGCAAGGGT |
| 461 | TGGGACAAGGATTGAAGCTGGCGTC | TGACGCCAGCTTCAATCCTTGTCCC |
| 462 | TTGTCGTTGCTCCCGAGTACCATTG | TCAATGGTACTCGGGAGCAACGACA |
| 463 | TGTTGTCCGAGACGTTTGTGTCAGC | TGCTGACACAAACGTCTCGGACAAC |
| 464 | TGCTGGTGAACACTCACGAACCGCT | TAGCGGTTCGTGAGTGTTCACCAGC |
| 465 | TGCAGACAGGGCAAATCGGTGCAAA | TTTTGCACCGATTTGCCCTGTCTGC |
| 466 | TCCCATCACAACGAGTGGCGACTTT | TAAAGTCGCCACTCGTTGTGATGGG |
| 467 | TGCTTCTACAGCTGGCGTGCTAGCG | TCGCTAGCACGCCAGCTGTAGAAGC |
| 468 | TGAATGTGTGCCGACCATTCTAGCC | TGGCTAGAATGGTCGGCACACATTC |
| 469 | TCCAGCGGAAGTTAGAGCTCTGTGG | TCCACAGAGCTCTAACTTCCGCTGG |
| 470 | TTTTTTACCGACCACTCCATGTCGG | TCCGACATGGAGTGGTCGGTAAAAA |
| 471 | TGCGGCTATGTGATGACGGCCTAGC | TGCTAGGCCGTCATCACATAGCCGC |
| 472 | TAGTACACGGGCGTGTTAGCGCTCC | TGGAGCGCTAACACGCCCGTGTACT |
| 473 | TTCCTGTGTGGTGGCGCACTCCCAC | TGTGGGAGTGCGCCACCACACAGGA |
| 474 | TCCAACTAACCAATCGCGCGGATGA | TTCATCCGCGCGATTGGTTAGTTGG |
| 475 | TAGTGAGTGACCAAGGCAGGAGCAA | TTTGCTCCTGCCTTGGTCACTCACT |
| 476 | TCATCTTTCGCGGAGTTTATTGCGG | TCCGCAATAAACTCCGCGAAAGATG |
| 477 | TCTTCGTCCGGTTAGTGCGACAGCA | TTGCTGTCGCACTAACCGGACGAAG |
| 478 | TCTCACGAAAACGTGGGCCCGAAAT | TATTTCGGGCCCACGTTTTCGTGAG |
| 479 | TCGCAGCAGCTGAACTCTAGCATTG | TCAATGCTAGAGTTCAGCTGCTGCG |
| 480 | TAGGAGACATACGCCCAAATGGTGC | TGCACCATTTGGGCGTATGTCTCCT |
| 481 | TATTGAGAACTCGTGCGGGAGTTTG | TCAAACTCCCGCACGAGTTCTCAAT |
| 482 | TCTCTTTGTAGGCCCAGGAGGAGCA | TTGCTCCTCCTGGGCCTACAAAGAG |
| 483 | TGCCGCAGGGTCGATAATTGGTCTA | TTAGACCAATTATCGACCCTGCGGC |
| 484 | TAAACGCCGCCCTGAGACTATTGGG | TCCCAATAGTCTCAGGGCGGCGTTT |
| 485 | TCTGAGTTGCCTGGAACGTTGGACT | TAGTCCAACGTTCCAGGCAACTCAG |
| 486 | TCGGATGGGTTGCAGAGTATGGGAT | TATCCCATACTCTGCAACCCATCCG |
| 487 | TCTGACCTTTGGGGGTTAGTGCGGT | TACCGCACTAACCCCCAAAGGTCAG |
| 488 | TGGAAATGAGAACCTTACCCCAGCG | TCGCTGGGGTAAGGTTCTCATTTCC |
| 489 | TAACGCATCGTCCGTCAACTCATCA | TTGATGAGTTGACGGACGATGCGTT |
| 490 | TTGGAGAGAGACTTCGGCCATTGTT | TAACAATGGCCGAAGTCTCTCTCCA |
| 491 | TTTGCGCTCATTGGATCTTGTCAGG | TCCTGACAAGATCCAATGAGCGCAA |
| 492 | TAGCGCGTTAAAGCACGGCAACATT | TAATGTTGCCGTGCTTTAACGCGCT |
| 493 | TAGCCAGTAAACTGTGGGCGGCTGT | TACAGCCGCCCACAGTTTACTGGCT |
| 494 | TCGACTGATGTGCAACCAGCAGCTG | TCAGCTGCTGGTTGCACATCAGTCG |
| 495 | TGGTTGCTCATACGACGAGCGAGTG | TCACTCGCTCGTCGTATGAGCAACC |
| 10 | TGTCCAACGCGCAACTCCGATTCAA | TTTGAATCGGAGTTGCGCGTTGGAC |
| 11 | TTTGCCGCACCGTCCGTCATCTCAA | TTTGAGATGACGGACGGTGCGGCAA |
| 498 | TAGAACCTCCGCGCCTCCGTAGTAG | TCTACTACGGAGGCGCGGAGGTTCT |
| 499 | TAAAGGAGCTTTCGCCCAACGTACC | TGGTACGTTGGGCGAAAGCTCCTTT |
| 500 | TAGTGATTGTGCCACTCCACAGCTC | TGAGCTGTGGAGTGGCACAATCACT |
| 501 | TGCGATCGTCGAGGGTTGAGCTGAA | TTTCAGCTCAACCCTCGACGATCGC |
| 502 | TGGGAGACAGCCATTATGGTCCTCG | TCGAGGACCATAATGGCTGTCTCCC |
| 503 | TGAGACGCTGTCACTCCGGCAGAAC | TGTTCTGCCGGAGTGACAGCGTCTC |
| 504 | TCCACCGGTCGCTTAAGATGCACTT | TAAGTGCATCTTAAGCGACCGGTGG |
| 505 | TCGGCATAACGTCCAGTCCTGGGAC | TGTCCCAGGACTGGACGTTATGCCG |
| 506 | TAAGCGGAACGGGTTATACCGAGGT | TACCTCGGTATAACCCGTTCCGCTT |
| 507 | TTGCACACTAGGTCCGTCGCTTGAT | TATCAAGCGACGGACCTAGTGTGCA |
| 508 | TAGGGAACCGCGTTCAAACTCAGTT | TAACTGAGTTTGAACGCGGTTCCCT |
| 509 | TGAATTACAACCACCCGCTCGTGTT | TAACACGAGCGGGTGGTTGTAATTC |
| 510 | TTTCAGTGCTCACGAAGCATGGATT | TAATCCATGCTTCGTGAGCACTGAA |
| 511 | TTTAGTTTGGCGTTGGGACTTCACC | TGGTGAAGTCCCAACGCCAAACTAA |
| 512 | TAATGCGACCTCGACGAGCCTCATA | TTATGAGGCTCGTCGAGGTCGCATT |
| 513 | TCCGAAACCGTTAACGTGGCGCACA | TTGTGCGCCACGTTAACGGTTTCGG |
| 514 | TTAAAGTAACAAGGCGACCTCCCGC | TGCGGGAGGTCGCCTTGTTACTTTA |
| 515 | TTAATGATTTTAGTCGCGGGGTGGG | TCCCACCCCGCGACTAAAATCATTA |
| 516 | TGGCTACTCTAAGTGCCCGCTCAGG | TCCTGAGCGGGCACTTAGAGTAGCC |
| 517 | TTGGCGGACGACTCAATATCTCACG | TCGTGAGATATTGAGTCGTCCGCCA |
| 518 | TGGGCGTTAGGCGTAATAGACCGTC | TGACGGTCTATTACGCCTAACGCCC |
| 519 | TGCCACCTTTAGACGGCGGCTCTAG | TCTAGAGCCGCCGTCTAAAGGTGGC |
| 520 | TGAGATGTGTAAACGTGCAGGCACC | TGGTGCCTGCACGTTTACACATCTC |
| 521 | TTAGCTCGTGGCCCTCCAAGCGTGT | TACACGCTTGGAGGGCCACGAGCTA |
| 522 | TGTGTCGGCGCTATTTGGCCTTACC | TGGTAAGGCCAAATAGCGCCGACAC |
| 523 | TCCAGGGAAGCAACTGGTTGCCATT | TAATGGCAACCAGTTGCTTCCCTGG |
| 524 | TTTCCGAAACTAAGCCAGAACCGCT | TAGCGGTTCTGGCTTAGTTTCGGAA |
| 525 | TGCAAACCCGGTAACCCGAGAGTTC | TGAACTCTCGGGTTACCGGGTTTGC |
| 526 | TGCAAATGGCGTCATGCACGAACGT | TACGTTCGTGCATGACGCCATTTGC |
| 527 | TAGTACTTTCGCGCCCAGTTTAGGG | TCCCTAAACTGGGCGCGAAAGTACT |
| 528 | TAAGATCTGCGAGGCATCCCGGCTT | TAAGCCGGGATGCCTCGCAGATCTT |
| 529 | TGCAAGTGTATCGCACAGTGCGATT | TAATCGCACTGTGCGATACACTTGC |
| 530 | TCCGACAAGGCCTCAATTCATTCTG | TCAGAATGAATTGAGGCCTTGTCGG |
| 531 | TGTCTCGTCTCAACTTTAAGGCGCG | TCGCGCCTTAAAGTTGAGACGAGAC |
| 532 | TATCCAGAGATCCGTTTTGCAGCGT | TACGCTGCAAAACGGATCTCTGGAT |
| 533 | TGTCACCAGGAGGGAAGTTTCACCC | TGGGTGAAACTTCCCTCCTGGTGAC |
| 534 | TTTCCGTCAGGCGGATCAACGGAAT | TATTCCGTTGATCCGCCTGACGGAA |
| 535 | TATGCCGGACACGCATTACACAGGC | TGCCTGTGTAATGCGTGTCCGGCAT |
| 536 | TTGGGCCGCTTGGCGCTTTCATAGA | TTCTATGAAAGCGCCAAGCGGCCCA |
| 537 | TCCTAGCGCGAGCTTTACTGACCAG | TCTGGTCAGTAAAGCTCGCGCTAGG |
| 538 | TTTGGCCAGGAATATGGTCTCGAGA | TTCTCGAGACCATATTCCTGGCCAA |
| 539 | TGTCTGCGGCCGACTTGCTATGCAT | TATGCATAGCAAGTCGGCCGCAGAC |
| 540 | TAACTTGCTCATTCTCAAGCCGACG | TCGTCGGCTTGAGAATGAGCAAGTT |
| 541 | TACGTCAGCGATTGTGGCGAAATAT | TATATTTCGCCACAATCGCTGACGT |
| 542 | TACGGCCTGCGTCAGCAGATGCATC | TGATGCATGTGCTGACGCAGGCCGT |
| 543 | TATACCTCCGCAGAACCATTCCGTT | TAACGGAATGGTTCTGCGGAGGTAT |
| 544 | TAGTTCGCGGTCCCACGATTCACTT | TAAGTGAATCGTGGGACCGCGAACT |
| 545 | TTGCTCAATTTGTGCAGAAAACGCC | TGGCGTTTTCTGCACAAATTGAGCA |
| 546 | TTTATCGCGAGAGACGACCGTGTCC | TGGACACGGTCGTCTCTCGCGATAA |
| 547 | TGACGCGACGTGAGTAGTGGAAGCG | TCGCTTCCACTACTCACGTCGCGTC |
| 548 | TATGGTAGGGGCATTGGGCTTTCCT | TAGGAAAGCCCAATGCCCCTACCAT |
| 549 | TCCAAATATAGCCGCGCGGAGACAT | TATGTCTCCGCGCGGCTATATTTGG |
| 550 | TGCAAACCCTGATTGAATCGTGCCC | TGGGCACGATTCAATCAGGGTTTGC |
| 551 | TTAGCGTCTTGCGTGAAACCATGGG | TCCCATGGTTTCACGCAAGACGCTA |
| 552 | TCCACCCCGACAGCGCTGGACTCTT | TAAGAGTCCAGCGCTGTCGGGGTGG |
| 553 | TACGAGCACTGAAGGCTGCTTTACG | TCGTAAAGCAGCCTTCAGTGCTCGT |
| 554 | TCATATCAGCGTCGTCTAGCTCGCG | TCGCGAGCTAGACGACGCTGATATG |
| 555 | TTGATCCCGGACCGGCTAGACTAAT | TATTAGTCTAGCCGGTCCGGGATCA |
| 556 | TGGCCCCGACACTACAGGGTAATCA | TTGATTACCCTGTAGTGTCGGGGCC |
| 557 | TGGCTCCAGGGCGAGATTATGAATG | TCATTCATAATCTCGCCCTGGAGCC |
| 558 | TCAAAATCCGATGGGCGGAAAATTA | TTAATTTTCCGCCCATCGGATTTTG |
| 559 | TCACAGGCGCATAGGGAGCAAGCTA | TTAGCTTGCTCCCTATGCGCCTGTG |
| 560 | TTAGCTATTGCCCCGATGGGCTACT | TAGTAGCCCATCGGGGCAATAGCTA |
| 561 | TTGGTACGCGGTCCATAGCAAGTCG | TCGACTTGCTATGGACCGCGTACCA |
| 562 | TGACGCTGTGGCTCGGAAACTGTTC | TGAACAGTTTCCGAGCCACAGCGTC |
| 563 | TCCTGGGTTCGCCGCGTGGTAACTG | TCAGTTACCACGCGGCGAACCCAGG |
| 564 | TTTCCCGCGTAGCCCAACAGCTATA | TTATAGCTGTTGGGCTACGCGGGAA |
| 565 | TTTCGCGGATTGCTGCCGCATAACA | TTGTTATGCGGCAGCAATCCGCGAA |
| 566 | TAAAAATGGCACCGAAGTTGAGGCA | TTGCCTCAACTTCGGTGCCATTTTT |
| 567 | TCATTCCGCGCGAGTTGAAATCCAG | TCTGGATTTCAACTCGCGCGGAATG |
| 568 | TACGCACGTTTTTTGGCACGGTTAA | TTTAACCGTGCCAAAAAACGTGCGT |
| 569 | TTGTCCATGACGTCGTTTCTCTGGT | TACCAGAGAAACGACGTCATGGACA |
| 570 | TTCTCAGTCGGACTCGTATGCCAGA | TTCTGGCATACGAGTCCGACTGAGA |
| 571 | TCTCCAAACGCACACATCAAGCATC | TGATGCTTGATGTGTGCGTTTGGAG |
| 572 | TTTCAACCAAGCGGGGTGTTCGTGA | TTCACGAACACCCCGCTTGGTTGAA |
| 573 | TGGTGTCGGAGGGTGGTGACCTCGA | TTCGAGGTCACCACCCTCCGACACC |
| 574 | TAGCGCTTTTGGTCATGATTTGCAA | TTTGCAAATCATGACCAAAAGCGCT |
| 575 | TCCGAGGACTTACGTCTGCCCAGGA | TTCCTGGGCAGACGTAAGTCCTCGG |
| 576 | TGCCCAATCCAGTTCTTATGCGCCC | TGGGCGCATAAGAACTGGATTGGGC |
| 577 | TCGGGTTAACCCACGCAAGTTATGA | TTCATAACTTGCGTGGGTTAACCCG |
| 578 | TTGATTAGCGCTCAATACACGCGTG | TCACGCGTGTATTGAGCGCTAATCA |
| 579 | TAAGGGCAGACCTTTGGTTCGACTG | TCAGTCGAACCAAAGGTCTGCCCTT |
| 580 | TGCGCCACAAGATTCACATGTCATT | TAATGACATGTGAATCTTGTGGCGC |
| 581 | TGCCATGTTCAAGGGCCTTTCGAAG | TCTTCGAAAGGCCCTTGAACATGGC |
| 582 | TCGCGGTGTTTTGTCTAGGTGCCGG | TCCGGCACCTAGACAAAACACCGCG |
| 583 | TCAACATTGTGGTGGCACTCCATCC | TGGATGGAGTGCCACCACAATGTTG |
| 584 | TCGATACGCGCCGGTTTGTTAAATC | TGATTTAACAAACCGGCGCGTATCG |
| 585 | TGGCTATAAACGTGCGGACTGCTCC | TGGAGCAGTCCGCACGTTTATAGCC |
| 586 | TTGGGTAAATCACTATTGCGCGGTT | TAACCGCGCAATAGTGATTTACCCA |
| 587 | TGTCTTCATCGGCCCGCGCAAGCTA | TTAGCTTGCGCGGGCCGATGAAGAC |
| 588 | TGCGACACACCCTGTACTCTGATGC | TGCATCAGAGTACAGGGTGTGTCGC |
| 589 | TGTAGCAGGGTCCGCAAGACCAAGC | TGCTTGGTCTTGCGGACCCTGCTAC |
| 590 | TTCGCCAACGCAGGGTAACTGCCAT | TATGGCAGTTACCCTGCGTTGGCGA |
| 591 | TACTCCGAAGCTTCGAGCGGCACGA | TTCGTGCCGCTCGAAGCTTCGGAGT |
| 12 | TCATCGTCCCTTTCGATGGGATCAA | TTTGATCCCATCGAAAGGGACGATG |
| 13 | TGCACGGGAGCTGACGACGTGTCAA | TTTGACACGTCGTCAGCTCCCGTGC |
| 594 | TATCATCCCACGGCAGAGTGAAGAG | TCTCTTCACTCTGCCGTGGGATGAT |
| 595 | TCGCTGGACTGGCCTATCCGAGTCG | TCGACTCGGATAGGCCAGTCCAGCG |
| 596 | TCGGTCTCAGCAACACTGTCGCAAA | TTTTGCGACAGTGTTGCTGAGACCG |
| 597 | TCGAACGTTCTCCGATGTAATGGCC | TGGCCATTACATCGGAGAACGTTCG |
| 598 | TATACCGTGCGACAAGCCCCTCTGA | TTCAGAGGGGCTTGTCGCACGGTAT |
| 599 | TAGCTCATTCCCGAGACGGAACACC | TGGTGTTCCGTCTCGGGAATGAGCT |
| 600 | TTTTCATGCGGCCGTTGCAAATCAT | TATGATTTGCAACGGCCGCATGAAA |
| 601 | TACTCGAACGGACGTTCAATTCCCA | TTGGGAATTGAACGTCCGTTCGAGT |
| 602 | TCTGCATGGTGTGGGTGAGACTCCC | TGGGAGTCTCACCCACACCATGCAG |
| 603 | TCCGCGAGTGTGGATGGCGTGTTGA | TTCAACACGCCATCCACACTCGCGG |
| 604 | TAATGTGTCGGTCCTAAGCCGGGTG | TCACCCGGCTTAGGACCGACACATT |
| 605 | TTAAGACGAGCCTGCACAGCTTGCG | TCGCAAGCTGTGCAGGCTCGTCTTA |
| 606 | TGGCGTGGGAGGATAAGACGATGTC | TGACATCGTCTTATCCTCCCACGCC |
| 607 | TTGCTCCATGTTAGGAACGCACCAC | TGTGGTGCGTTCCTAACATGGAGCA |
| 608 | TCGGTGTTGGTCGGACTGACGACTG | TCAGTCGTCAGTCCGACCAACACCG |
| 609 | TCCGCGCGTATCTATCAGATCTGGG | TCCCAGATCTGATAGATACGCGCGG |
| 610 | TAAAGCATGCTCCACCTGGAGCGAG | TCTCGCTCCAGGTGGAGCATGCTTT |
| 611 | TACTTGCATCGCTGGGTAGATCCGG | TCCGGATCTACCCAGCGATGCAAGT |
| 612 | TTGCTTACGCAGTGGATTGGTCAGA | TTCTGACCAATCCACTGCGTAAGCA |
| 613 | TATGCAGATGAACAAATCGCCGAAT | TATTCGGCGATTTGTTCATCTGCAT |
| 614 | TGCAATTCTGGGCCATGTATTCGTC | TGACGAATACATGGCCCAGAATTGC |
| 615 | TAGGGTTCCTTACGCGTCGACATGG | TCCATGTCGACGCGTAAGGAACCCT |
| 616 | TGTGGAGCTAATCGCGAGCCTCAGA | TTCTGAGGCTCGCGATTAGCTCCAC |
| 617 | TTCGTAGTCTCACCGGCAATGATCC | TGGATCATTGCCGGTGAGACTACGA |
| 618 | TTTATAGCAGTGCGCCAATGCTTCG | TCGAAGCATTGGCGCACTGCTATAA |
| 619 | TCGAACAGTGCTGTCCGTCGCTCAA | TTTGAGCGACGGACAGCACTGTTCG |
| 620 | TTCCGCGTGGACTGTTAGACGCTAT | TATAGCGTCTAACAGTCCACGCGGA |
| 621 | TCATTAGCCCGCTGTCGGTAACTGT | TACAGTTACCGACAGCGGGCTAATG |
| 622 | TGGAAAGAAACTCAGACGCGCAATG | TCATTGCGCGTCTGAGTTTCTTTCC |
| 623 | TCGACTCGCTGGACAGGAGAATCGT | TACGATTCTCCTGTCCAGCGAGTCG |
| 624 | TCATGATCCTCTGTTTCACCCGCGG | TCCGCGGGTGAAACAGAGGATCATG |
| 625 | TGGCGTAGCGCTCTAAAAGCTTCGG | TCCGAAGCTTTTAGAGCGCTACGCC |
| 626 | TAGTGATGCCATCAGGCCCGTATAC | TGTATACGGGCCTGATGGCATCACT |
| 627 | TTATGGAAAGGGCAACAGCGCTATC | TGATAGCGCTGTTGCCCTTTCCATA |
| 628 | TCTGTGGTTGATGGAGGATCCACAC | TGTGTGGATCCTCCATCAACCACAG |
| 629 | TACTCGCTGGAATTTGCGCTGACAC | TGTGTCAGCGCAAATTCCAGCGAGT |
| 630 | TCAGGCCCGAACCACGCGGTTACAG | TCTGTAACCGCGTGGTTCGGGCCTG |
| 631 | TGGCGCAATGGGCGCATAAATACTA | TTAGTATTTATGCGCCCATTGCGCC |
| 632 | TGGTCAATTCGCGCTACATGCCCTA | TTAGGGCATGTAGCGCGAATTGACC |
| 633 | TGATGGTGGACTGGAGCCCTTCCGC | TGCGGAAGGGCTCCAGTCCACCATC |
| 634 | TCCGCGCATAGCGCAATAGGGGAGA | TTCTCCCCTATTGCGCTATGCGCGG |
| 635 | TTCTTCTGGCTGTCCGGCACCCGAA | TTTCGGGTGCCGGACAGCCAGAAGA |
| 636 | TGCGTTCGCAATTCACGGGCCCTTA | TTAAGGGCCCGTGAATTGCGAACGC |
| 637 | TTCGTTTCGGCCTTGGAGAGTATCG | TCGATACTCTCCAAGGCCGAAACGA |
| 638 | TAGGTGCAAGTGCAAGGCGAGAGGC | TGCCTCTCGCCTTGCACTTGCACCT |
| 639 | TCGCCAGTTTCGATGGCTGACGTTT | TAAACGTCAGCCATCGAAACTGGCG |
| 640 | TGCTTTACCGCCGATCCCAGATATC | TGATATCTGGGATCGGCGGTAAAGC |
| 641 | TGTGCTTGACGAAGAGGCGAAATGT | TACATTTCGCCTCTTCGTCAAGCAC |
| 642 | TCAGTCCGTGCGCTTCATGTCCTCA | TTGAGGACATGAAGCGCACGGACTG |
| 643 | TTACGCGTAAGAGCCTACCCTCGCG | TCGCGAGGGTAGGCTCTTACGCGTA |
| 644 | TGGCGAGTCTTGTGGGGACATGTGT | TACACATGTCCCCACAAGACTCGCC |
| 645 | TCCAAAGCGAAGCGAGCGTGTCTAT | TATAGACACGCTCGCTTCGCTTTGG |
| 646 | TGCCGTAGGTTGCTCTTCACCGAAC | TGTTCGGTGAAGAGCAACCTACGGC |
| 647 | TAAATCCGCGATGTGCCGTGAGGCT | TAGCCTCACGGCACATCGCGGATTT |
| 648 | TGGCTTCGCACCCGTACCAATTTAG | TCTAAATTGGTACGGGTGCGAAGCC |
| 649 | TTGTAGAGTCCCACGTAGCCGGCAT | TATGCCGGCTACGTGGGACTCTACA |
| 650 | TCACTAGTCTGGGGCAAGGTGCATT | TAATGCACCTTGCCCCAGACTAGTG |
| 651 | TTGTACTCGGCAGGCGCAATAGATT | TAATCTATTGCGCCTGCCGAGTACA |
| 652 | TAACGGGTATCGGAAGCGTAAAAGC | TGCTTTTACGCTTCCGATACCCGTT |
| 653 | TCGGACTGCCCGTTTGCAAGTTGAG | TCTCAACTTGCAAACGGGCAGTCCG |
| 654 | TATCGTTCAGCACTGGAGCCCGTAA | TTTACGGGCTCCAGTGCTGAACGAT |
| 655 | TATGCATCGAACTAGTCGTGACGGC | TGCCGTCACGACTAGTTCGATGCAT |
| 656 | TTTCCAGGCATTAAGGAGAGGGAGC | TGCTCCCTCTCCTTAATGCCTGGAA |
| 657 | TGTGCGACATCTACTCCACGATCCC | TGGGATCGTGGAGTAGATGTCGCAC |
| 658 | TCTCATCGTCCTAACACGAGAGCCC | TGGGCTCTCGTGTTAGGACGATGAG |
| 659 | TAATGGCACTTCGGCGGTGATGCAA | TTTGCATCACCGCCGAAGTGCCATT |
| 660 | TCCGTGGGAGGGAATCCAACCGAGG | TCCTCGGTTGGATTCCCTCCCACGG |
| 661 | TAAATTCTCGTTGGTGACGGCTCAT | TATGAGCCGTCACCAACGAGAATTT |
| 662 | TTTGCTCTTATCCTTGTCCTGGGCG | TCGCCCAGGACAAGGATAAGAGCAA |
| 663 | TTTAAGGATCAGGCGGAGCTTGCAG | TCTGCAAGCTCCGCCTGATCCTTAA |
| 664 | TCGCGACTAAGGTGCTGCAACTCGA | TTCGAGTTGCAGCACCTTAGTCGCG |
| 665 | TGCTCGATTTCACGGCCCGTTGTTC | TGAACAACGGGCCGTGAAATCGAGC |
| 666 | TAGCAGAGTGCGTTGCAGAGGCTAA | TTTAGCCTCTGCAACGCACTCTGCT |
| 667 | TTGGAGGTGAGGACGACGTGCACTA | TTAGTGCACGTCGTCCTCACCTCCA |
| 668 | TAACCGTTTAGGGTACATTCGCGGT | TACCGCGAATGTACCCTAAACGGTT |
| 669 | TTATGATCGCTCGGCTCACAGTTTG | TCAAACTGTGAGCCGAGCGATCATA |
| 670 | TGACTTTTTGCGGAAACGTCATGGT | TACCATGACGTTTCCGCAAAAAGTC |
| 671 | TTGTCGGTTATTCCACCTGCAAGGA | TTCCTTGCAGGTGGAATAACCGACA |
| 672 | TCTATGGTTTGCACTGCGCCGTCGA | TTCGACGGCGCAGTGCAAACCATAG |
| 673 | TAGCAGGGAAATTCAATCGTTCGCA | TTGCGAACGATTGAATTTCCCTGCT |
| 674 | TCCTAACCGAGCGCTTAGCATTTCC | TGGAAATGCTAAGCGCTCGGTTAGG |
| 675 | TCCCGACCCTAACTCGCATTGAATA | TTATTCAATGCGAGTTAGGGTCGGG |
| 676 | TTTGCTTAATGGTGACGCCACGGAT | TATCCGTGGCGTCACCATTAAGCAA |
| 677 | TGATGCTCGCCGTGTTTAGTTCACG | TCGTGAACTAAACACGGCGAGCATC |
| 678 | TTCGGATGACGAGTTTCCATGACGG | TCCGTCATGGAAACTCGTCATCCGA |
| 679 | TATGCGGTCTACTTTCTCGATCGGG | TCCCGATCGAGAAAGTAGACCGCAT |
| 680 | TTTGCGAGGCTAAGCACACGGTAAA | TTTTACCGTGTGCTTAGCCTCGCAA |
| 681 | TAACTTAATTACCGCCTCTGGCGCC | TGGCGCCAGAGGCGGTAATTAAGTT |
| 682 | TGTGACCGCGAACTTGTTCCGACAG | TCTGTCGGAACAAGTTCGCGGTCAC |
| 683 | TTGCGGATTACCGATTCGCTCTTAA | TTTAAGAGCGAATCGGTAATCCGCA |
| 684 | TTGATAGGGGGCCACGTTGATCAGA | TTCTGATCAACGTGGCCCCCTATCA |
| 685 | TTCGCTCCGTAGCGATTCATCGTAG | TCTACGATGAATCGCTACGGAGCGA |
| 686 | TTGTCAGCTGGTAGCCTCCGTTTGA | TTCAAACGGAGGCTACCAGCTGACA |
| 687 | TAGCGTCGCATGACGCTTACGGCAC | TGTGCCGTAAGCGTCATGCGACGCT |
| 14 | TAGACGCACCGCAACAGGCTGTCAA | TTTGACAGCCTGTTGCGGTGCGTCT |
| 15 | TCGTGTAGGGGTCCCGTGCTGTCAA | TTTGACAGCACGGGACCCCTACACG |
| 690 | TGTCGCATTCTGCACTGGCTTCGCC | TGGCGAAGCCAGTGCAGAATGCGAC |
| 691 | TTGATTAGGTGCGGTCCCGTAGTCC | TGGACTACGGGACCGCACCTAATCA |
| 692 | TAAGGGACCTTGGGTGACGGCGAGA | TTCTCGCCGTCACCCAAGGTCCCTT |
| 693 | TTCAAATGGCCACCGCGTGTCATTC | TGAATGACACGCGGTGGCCATTTGA |
| 694 | TCTCCGACGACCAATAAATAGCCGC | TGCGGCTATTTATTGGTCGTCGGAG |
| 695 | TGGCTATTCCCGTAGAGAGCGTCCA | TTGGACGCTCTCTACGGGAATAGCC |
| 696 | TTGGATAACCTCTCGGTCCATCCAC | TGTGGATGGACCGAGAGGTTATCCA |
| 697 | TGACCGCTGTACGGGAGTGTGCCTT | TAAGGCACACTCCCGTACAGCGGTG |
| 698 | TGCCACAGAGTTTTAGCAGGGACCC | TGGGTCCCTGCTAAAACTCTGTGGC |
| 699 | TCCCACGCTTTCCGACCACTGACCT | TAGGTCAGTGGTCGGAAAGCGTGGG |
| 700 | TCATTGACACAATGCGGGGACTGAT | TATCAGTCCCCGCATTGTGTCAATG |
| 701 | TAGCCACTCGACAGGGTTCCAAAGC | TGCTTTGGAACCCTGTCGAGTGGCT |
| 702 | TCAGGATGAGCAAAGCGACTCTCCA | TTGGAGAGTCGCTTTGCTCATCCTG |
| 703 | TCAAGGTATGGTCTGGGGCCTAAGG | TGCTTAGGCCCCAGACCATACCTTG |
| 704 | TGGTGTTCGGCCTAAACTCTTTCGG | TCCGAAAGAGTTTAGGCCGAACACC |
| 705 | TTTTAGTCGGACCCTGTGGCAATTC | TGAATTGCCACAGGGTCCGACTAAA |
| 706 | TCACACGTTTCCGACCAGCCTGAAC | TGTTCAGGCTGGTCGGAAACGTGTG |
| 707 | TCTGGACGAACTGGCTTCCTCGTAC | TGTACGAGGAAGCCAGTTCGTCCAG |
| 708 | TTTCACAATCCGCCGAAAACTGACC | TGGTCAGTTTTCGGCGGATTGTGAA |
| 709 | TAACAGGATATCCGCGATCACGACA | TTGTCGTGATCGCGGATATCCTGTT |
| 710 | TTACGTCGGATCCATTGCGCCGAGT | TACTCGGCGCAATGGATCCGACGTA |
| 711 | TCATGGATCTCTCGGTTTGATCGCC | TGGCGATCAAACCGAGAGATCCATG |
| 712 | TAGCCAGGCGCGTATATACGCTCGG | TCCGAGCGTATATACGCGCCTGGCT |
| 713 | TATTTGGCACGTGTCGTGCCATGTT | TAACATGGCACGACACGTGCCAAAT |
| 714 | TCCGCGTTGCACCACTTTGAGGTGC | TGCACCTCAAAGTGGTGCAACGCGG |
| 715 | TTTGGACGTGACAAGCATGGCGCTC | TGAGCGCCATGCTTGTCACGTCCAA |
| 716 | TCTGAATCGCGCAAGTAAATGGGGG | TCCCCCATTTACTTGCGCGATTCAG |
| 717 | TGATAAGGTCCACCAGATTGCGCGC | TGCGCGCAATCTGGTGGACCTTATC |
| 718 | TCTAACAATTGCCAACCGGGACGGC | TGCCGTCCCGGTTGGCAATTGTTAG |
| 719 | TGGTAACCTGGGTGCTTGCAGGTTA | TTAACCTGCAAGCACCCAGGTTACC |
| 720 | TATCGGAGCCACCATTCGCATTGGG | TCCCAATGCGAATGGTGGCTCCGAT |
| 721 | TGTGAACTGGCTTGCCCCAGGATTA | TTAATCCTGGGGCAAGCCAGTTCAC |
| 722 | TAGGCGATAGCATGGTCCCATATGA | TTCATATGGGACCATGCTATCGCCT |
| 723 | TAACGGTATCGTGGCTAATGCACGA | TTCGTGCATTAGCCACGATACCGTT |
| 724 | TAGTAGTGGTCCTCCAGATCGGCAA | TTTGCCGATCTGGAGGACCACTACT |
| 725 | TCCGTTGAATTGGACGGGAGGTTAG | TCTAACCTCCCGTCCAATTCAACGG |
| 726 | TGCATAAGTGCGGCATCGCGAAGGG | TCCCTTCGCGATGCCGCACTTATGC |
| 727 | TCGACAAGATGCAGCTGCTACATGC | TGCATGTAGCAGCTGCATCTTGTCG |
| 728 | TTCGCAGTGATTCCCGACCGATAAG | TCTTATCGGTCGGGAATCACTGCGA |
| 729 | TCAAGGCGAGTCCACTCGAGGGGAC | TGTCCCCTCGAGTGGACTCGCCTTG |
| 730 | TGCAACTTGCACGGCATAAGTGGGC | TGGCCACTTATGCCGTGCAAGTTGC |
| 731 | TTCCGAGCTTGACGTTCGCGACGTC | TGACGTCGCGAACGTCAAGCTCGGA |
| 732 | TAGCGCTGGGCTGTGCTGCCATCTC | TGAGATGGCAGCACAGCCCAGCGCT |
| 733 | TTTCATGTCGCTGAGTAACCCTCGC | TGCGAGGGTTACTCAGCGACATGAA |
| 734 | TCGAACCGCTAATGCCCATTGTCAG | TCTGACAATGGGCATTAGCGGTTCG |
| 735 | TCACGGAAGGTGGGACAAATCGCCG | TCGGCGATTTGTCCCACCTTCCGTG |
| 736 | TCACAGATGGAGACAAACGCGCCTT | TAAGGCGCGTTTGTCTCCATCTGTG |
| 737 | TTTTTCGCAACTCGCTCCATAACCC | TGGGTTATGGAGCGAGTTGCGAAAA |
| 738 | TACGTTACGTTTCCGGCGCCTCTAA | TTTAGAGGCGCCGGAAACGTAACGT |
| 739 | TTATCGGATTGCGTGGGTTTCAATC | TGATTGAAACCCACGCAATCCGATA |
| 740 | TCTTCCACAATTGTCTGCGACGCAC | TGTGCGTCGCAGACAATTGTGGAAG |
| 741 | TTGCACAAAGGTATGGCTGTCCGGC | TGCCGGACAGCCATACCTTTGTGCA |
| 742 | TTCCGATGCCAGTCCCATCTTAAGA | TTCTTAAGATGGGACTGGCATCGGA |
| 743 | TCTGAAACCGTGCGAATCGAGGTGA | TTCACCTCGATTCGCACGGTTTCAG |
| 744 | TCGGTGTTCCGCGTGTCGAAAAAAT | TATTTTTTCGACACGCGGAACACCG |
| 745 | TTCTAGCAGGCCTTTTGAATCGCCA | TTGGCGATTCAAAAGGCCTGCTAGA |
| 746 | TGAGTCACCTCTGAGACGGACGCCA | TTGGCGTCCGTCTCAGAGGTGACTC |
| 747 | TTCTTCTGTCATCCTGCAGCAGCAT | TATGCTGCTGCAGGATGACAGAAGA |
| 748 | TGCGGATGAAACCTGAAAGGGGCCT | TAGGCCCCTTTCAGGTTTCATCCGC |
| 749 | TGGGGCCCCAAACTGGTATCAAGCC | TGGCTTGATACCAGTTTGGGGCCCC |
| 750 | TGCATTGGCTTCGGATTCTCCTACA | TTGTAGGAGAATCCGAAGCCAATGC |
| 751 | TAGGCGGCCCAACTGTGAGGTCTTG | TCAAGACCTCACAGTTGGGCCGCCT |
| 752 | TACACCATGTGCTCCGCGCTGCAGT | TACTGCAGCGCGGAGCACATGGTGT |
| 753 | TACGATGAACATGAATCGGGAGTCG | TCGACTCCCGATTCATGTTCATCGT |
| 754 | TCTGCATCCCTGTAGCAGCGCTCCG | TCGGAGCGCTGCTACAGGGATGCAG |
| 755 | TGTGCCGTATTTCGACCTGTGCGTT | TAACGCACAGGTCGAAATACGGCAC |
| 756 | TGCAGTGCGCACTTCAGTTCAAAAG | TCTTTTGAACTGAAGTGCGCACTGC |
| 757 | TGCGATTTTAAGCGATGCCTTGACG | TCGTCAAGGCATCGCTTAAAATCGC |
| 758 | TTAGGTGACCTAGGCTTGCTTGCGG | TCCGCAAGCAAGCCTAGGTCACCTA |
| 759 | TCTGGATACCTTGCCTGTGCGGCGC | TGCGCCGCACAGGCAAGGTATCCAG |
| 760 | TCCCCTTACGGCTCGTCGTCTATGC | TGCATAGACGACGAGCCGTAAGGGG |
| 761 | TGCGCTTGCCCGATGCGATGCATTA | TTAATGCATCGCATCGGGCAAGCGC |
| 762 | TTTTCTGTAAGCGGCCTGGGGTTCA | TTGAACCCCAGGCCGCTTACAGAAA |
| 763 | TGGCTGAGGTGAGCGGTAAGGATGA | TTCATCCTTACCGCTCACCTCAGCC |
| 764 | TTCTTGGCCTCCCCGATCTAATTTG | TCAAATTAGATCGGGGAGGCCAAGA |
| 765 | TGGAGGTAACGCCGTGTACGTAGGA | TTCCTACGTACACGGCGTTACCTCC |
| 766 | TGTAATCCATTTGTGGCTGCGTCAA | TTTGACGCAGCCACAAATGGATTAC |
| 767 | TCAAACCCATTCCAGCAGACGCCTG | TCAGGCGTCTGCTGGAATGGGTTTG |
| 768 | TTAGGAGGAATTTGGCATGCGGGCG | TCGCCCGCATGCCAAATTCCTCCTA |
| 769 | TATAGGTAGGATGTGCCCGGCGTTG | TCAACGCCGGGCACATCCTACCTAT |
| 770 | TGCAAGTGCTTAGCTCGTCAGCCTC | TGAGGCTGACGAGCTAAGCACTTGC |
| 771 | TCTGGCTGTGTCGCATCTCGTTAAC | TGTTAACGAGATGCGACACAGCCAG |
| 772 | TCTAACGTCGTCTCGCGCAATCACT | TAGTGATTGCGCGAGACGACGTTAG |
| 773 | TTTTTCATAAACGTTGTCCCCGAGC | TGCTCGGGGACAACGTTTATGAAAA |
| 774 | TAGCAGGAGGACGAACCTCCGCTCC | TGGAGCGGAGGTTCGTCCTCCTGCT |
| 775 | TTTCAAGCACCATCGTGCAATCCAA | TTTGGATTGCACGATGGTGCTTGAA |
| 776 | TAGCGTCGCCAGTGATCGCTAGTGG | TCCACTAGCGATCACTGGCGACGCT |
| 777 | TTACATTCCCTGCCTCCGTGGGCTT | TAAGCCCACGGAGGCAGGGAATGTA |
| 778 | TCGCTTCGCGTATTCAGTAGCGGTT | TAACCGCTACTGAATACGCGAAGCG |
| 779 | TTCGGACGCGTCGACACTCATTATA | TTATAATGAGTGTCGACGCGTCCGA |
| 780 | TTCTGAGCAGGCCAGCGCTCCAGCT | TAGCTGGAGCGCTGGCCTGCTCAGA |
| 781 | TTTGAATTGCCAAGCCCTGAAAGCC | TGGCTTTCAGGGCTTGGCAATTCAA |
| 782 | TAGTTTTCGCCTTGATGCGTCGGTG | TCACCGACGCATCAAGGCGAAAACT |
| 783 | TGTTTCATAGGCCACGCGTGCTAAA | TTTTAGCACGCGTGGCCTATGAAAC |
| 16 | TCATCGCTGCAAGTACCGCACTCAA | TTTGAGTGCGGTACTTGCAGCGATG |

We claim:

1. An oligonucleotide array comprising an array of at least 25 different addresses, each address comprising a different capture probe selected from the group consisting of the sequences set forth in Table 1, Table 2, Table 3 and Table 4.

2. An array according to claim 1, wherein said capture probes are microspheres.

3. An array according to claim 1 or 2 wherein said array is a liquid array.

4. An array according to claim 1 or 2, wherein said array further comprises a solid support.

5. An array according to claim 1, wherein said addresses are microspheres and wherein said solid support comprises wells into which said microspheres are individually distributed.

6. An array according to claim 1, wherein each address is a different known location, and said wherein each capture probe is attached to one of said known locations.

7. An array according to claim 1, wherein said array comprises at least 50 different addresses, each address comprising a different capture probe selected from the group consisting of the sequences set forth in Table 1, Table 2, Table 3 and Table 4.

8. An array according to claim 1 wherein said array comprises at least 100 different addresses, each address comprising a different capture probe selected from the group consisting of the sequences set forth in Table 1, Table 2, Table 3 and Table 4.

9. A kit comprising at least twenty-five nucleic acids selected from the group consisting of sequences substantially complementary to the sequences set forth in Table I, Table II, Table III and Table IV or their complement.

10. A kit according to claim 9, wherein said kit comprises at least 50 nucleic acids selected from the group consisting of the sequences substantially complementary to the sequences set forth in Table I, Table II, Table III and Table IV or their complement.

11. A kit according to claim 9, wherein said kit comprises at least 100 nucleic acids selected from the group consisting of the sequences substantially complementary to the sequences set forth in Table I, Table II, Table III and Table IV or their complement.

12. A kit according to claim 9, wherein said nucleic acids further comprise at least a first universal priming sequence.

13. A kit according to claim 9, wherein said nucleic acid sequence further comprises a sequence substantially complementary to a target domain.

14. A method of immobilizing a target nucleic acid sequence, said method comprising:
a) attaching a first adapter nucleic acid to a first target nucleic acid sequence to form a modified first target nucleic acid sequence, wherein said first adapter nucleic acid comprises a sequence substantially complementary to a sequence selected from the sequences set forth in Table I, Table II, Table III, and Table IV;
b) contacting said modified first target nucleic acid sequence with an array comprising an array of at least 25 different addresses, each address comprising a different capture probe selected from the group consisting of the sequences set forth in Table 1, Table 2, Table 3 and Table 4, whereby said target nucleic acid sequence is immobilized.

15. A method of detecting a target nucleic acid sequence, said method comprising:
a) attaching a first adapter nucleic acid to a first target nucleic acid sequence to form a modified first target nucleic acid sequence, wherein said first adapter nucleic acid comprises a sequence substantially complementary to a sequence selected from the sequences set forth in Table I, Table II, Table III, and Table IV;
b) contacting said modified first target nucleic acid sequence with an array comprising: an array of at least 25 different addresses, each address comprising a different capture probe selected from the group consisting of the sequences set forth in Table 1, Table 2, Table 3 and Table 4; and
c) detecting the presence of said modified first target nucleic acid sequence.

16. A method of detecting a target nucleic acid, said method comprising:
a) hybridizing a first adapter probe with a first target nucleic acid, said first adapter probe comprising a first domain that is complementary to said first target nucleic acid and a second domain, said second domain comprising a first sequence substantially complementary to a selected from the group consisting of the sequences set forth in Table I, Table II, Table III and Table IV to form a first hybridization complex;
b) contacting said first hybridization complex with an enzyme such that when said first domain of said adapter probe is perfectly complementary with said first target nucleic acid, said first adapter probe is altered resulting in a modified first adapter probe;
c) contacting said modified first adapter probe with a population of microspheres comprising at least a first subpopulation comprising a first capture probe, such that said first capture probe and said modified first adapter probe form a second hybridization complex; and
d) detecting the presence of said modified first adapter probe as an indication of the presence of said target nucleic acid.
